(19) United States
(12) Patent Application Publication
(10) Pub. No.: US 2015/0231240 A1
(43) Pub. Date: Aug. 20, 2015

(54) ANTI-C16ORF54 ANTIBODIES AND METHODS OF USE THEREOF

(71) Applicant: IGENICA, INC., Burlingame, CA (US)

(72) Inventors: Guoqing Chen, Palo Alto, CA (US); Sun Young Kim, Foster City, CA (US); Jan-Willem Theunissen, Foster City, CA (US); Edward Thein-Htun Van Der Horst, Palo Alto, CA (US); David Jackson, Belmont, CA (US); Edward Ha, San Francisco, CA (US); John Lippincott, San Mateo, CA (US)

(73) Assignee: IGENICA BIOTHERAPEUTICS, INC., Burlingame, CA (US)

(21) Appl. No.: 14/434,394

(22) PCT Filed: Oct. 9, 2013
(86) PCT No.: PCT/US13/64147

§ 371 Date: Apr. 8, 2015

Related U.S. Application Data
(60) Provisional application No. 61/711,699, filed on Oct. 9, 2012.
(60) Provisional application No. 61/834,870, filed on Jun. 13, 2013.

(51) Int. Cl. A61K 39/395; G01N 33/574; A61K 45/06; A01K 67/027; C07K 16/30; A61K 47/48

(52) U.S. Cl. CPC A61K 39/39558; C07K 16/30; A61K 47/48569; A61K 45/06; A01K 67/0275; G01N 33/57426; C07K 2317/76; A01K 2267/01; G01N 2333/705

(57) ABSTRACT

The invention provides antibodies that bind C16orf54, including antibody-drug conjugates comprising the antibodies, and methods of use of the antibodies and the antibody-drug conjugates, including for the diagnosis and treatment of cancers.

### CROSS-REFERENCE

This application claims the benefit of priority of U.S. Provisional application Ser. No. 61/711,699, filed Oct. 9, 2012, and U.S. Provisional application Ser. No. 61/834,870, filed Jun. 13, 2013, each of which are incorporated herein by reference in its entirety.

### FIELD OF THE INVENTION

The present invention relates generally to anti-C16orf54 antibodies and to methods of using such antibodies.

### BACKGROUND

Hematologic cancers, also referred to as liquid tumors, are cancers of the blood, bone marrow and lymph nodes, and include leukemia, lymphoma and myeloma. Leukemias are cancers of the blood-forming tissues characterized by distorted proliferation and development of leukocytes and their precursors in the blood and bone marrow. Leukemias are typically classified as either chronic (slowly progressing) or acute (rapidly progressing). Leukemias are further classified based upon the type of white blood cell that is affected, either lymphoid cells (lymphoid, lymphocytic or lymphoblastic leukemia) or myeloid cells (myeloid, myelogenous, myeloblastic, or granulocytic leukemia). The four main types of leukemia are acute lymphoblastic leukemia (ALL), chronic lymphocytic leukemia (CLL), acute myelogenous leukemia (AML), and chronic myelogenous leukemia (CML). Lymphomas are cancers that start in the lymph system, mainly the lymph nodes. The two main types of lymphoma are Hodgkin lymphoma, which spreads in an orderly manner from one group of lymph nodes to another, and non-Hodgkin lymphoma, which spreads through the lymphatic system in a non-orderly manner. Myeloma (multiple myeloma or plasma cell myeloma) is a cancer of the plasma cells, characterized by an accumulation of malignant plasma cells in the bone marrow, bone destruction, and progressive bone failure. Solid tumors refer to a solid mass of cancer cells that grow in organ systems and can occur anywhere in the body, for example, breast cancer or pancreatic cancer. Two types of solid tumors are seen in adults: epithelial tumors and sarcomas. Epithelial tumors, which can also be called carcinomas, occur in the lining (epithelium) that is on the outside or inside of the organ. Sarcomas are also called “connective tissue tumors” because they occur in the tissue that keeps the organs together.

Chronic lymphocytic leukemia (CLL) is the most common form of leukemia in the western world. CLL is typically a disease of the elderly, with a median age at diagnosis of 70. It is most often detected before the presentation of any symptoms, by the detection of a high white blood cell count in a routine blood test. Diagnosis of CLL is confirmed by the detection of a clonal population of B lymphocytes that express the characteristic cell surface markers CD5 and CD23. Small lymphocytic lymphoma is essentially the same disease as CLL, with slightly different manifestations.

The staging of CLL is based upon the Rai or Binet systems. Other parameters predictive of high-risk CLL include low levels of somatic hypermutations in the immunoglobulin V_{H} gene region, high expression levels of ZAP70 and CD38, and the presence of genomic aberrations defined as 17p and 11q deletions. Patients at earlier stages (Rai 0-II, Binet A) are typically monitored without therapy unless they show signs of disease progression. Patients at intermediate (Rai III and IV, Binet B and C)) usually benefit from the initiation of treatment.

Treatments of CLL include monotherapy with purine analogs, with fludarabine, pentostatin and cladribine being the purine analogs currently used in CLL. Since the 1990s, combination chemotherapies have been used, typically involving purine analogs combined with alkylating agents, such as bendamustine hydrochloride or cyclophosphamide. The combination of fludarabine and cyclophosphamide (FC) is the most robust of these combined chemotherapies. Chemotherapy may also be combined with therapeutic monoclonal antibodies. Rituximab, a chimeric anti-CD20 monoclonal antibody, has proven highly effective in combination with fludarabine and cyclophosphamide (FCR). Alemtuzumab, a humanized anti-CD52 monoclonal antibody, is effective in treating relapsed or refractory CLL when used as a single agent, and has also been tested in combination therapies with rituximab and FCR. Additional candidates for chemoimmunotherapy of CLL are the newly developed humanized anti-CD20 antibodies ofatumumab, obinutuzumab, veltuzumab, and ocrelizumab, and lumiliximab, a primatized anti-CD23 antibody. Other new agents being tested in CLL treatment include lenalidomide, an immunomodulatory agent, flavopiridol, a synthetic flavon, and the Bcl2 antagonists oblimersen and ABT-263.

For review of CLL diagnosis and treatment, see Hallek, M. et al. (2008) Blood 111: 5446-5456; Hallek, M. et al. (2005) Hematology 285-291; Hallek, M. and Pflug, N. (2010) Annals of Oncology 21 (Suppl. 7): vii154-vii164); and Nabhan, C. and Kay. N.E. (2011) Clinical Medicine Insights: Oncology 5: 45-53.

Approximately 300,000 patients in the world are diagnosed annually with acute myeloid leukemia (AML), with a median age of about 67 years. Despite advances in therapy, most patients die of their disease. AML is grouped into several subtypes, based upon morphology and surface markers of the cancerous cells, using either the WHO or the French-American-British (FAB) classification system. Other prognostic indicators of newly diagnosed AML are cytogenetics, with three risk categories (favorable, intermediate, and poor risk) based upon characteristic chromosomal abnormalities. Genetic mutations conferring improved (NPM1 or CEBPA) or inferior (FLT-3) outcomes have also recently been identified.

Treatment for AML generally includes two stages, remission induction therapy, followed by consolidation therapy with either 1-4 cycles of chemotherapy or stem cell transplantation. For a subtype of AML, acute promyelocytic leukemia, over 75% of patients can be cured with a combination of anthracycline-based therapy, all-trans retinoic acid, and arsenic trioxide. For all other types of AML, the drugs for remission and consolidation therapy are typically cytosine arabinoside (ara-C; cytarabine) combined with an anthracycline or anthracenedione, such as daunorubicin, adriamycin, idarubicin, or mitoxantrone.

Other new agents being tested in AML treatment include gemtuzumab ozogamicin (Mylotarg), an antibody-drug conjugate comprising an anti-CD33 antibody linked to calicheamicin, lenalidomide, an immunomodulatory agent, hypomethylating agents such as azacytidine or decitabine, clofarabine, a nucleoside analog, and FLT3 inhibitors such as midostaurin, sorafenib and AC220. For review of AML diagnosis and treatment, see Rowe, J. M. (2009) Hematology 2009:396-405; Roboz, G. J. (2011) Hematology 2011: 43-50; and Lin, T. L and Levy, M. Y (2012) Clinical Medicine Insights: Oncology 6: 205-217.

Tumor associated antigens are cell surface molecules that are more highly expressed on tumor cells than on normal cells, and thus can be used to immunologically distinguish between cancer and normal cells. These tumor associated antigens may be used as diagnostic or prognostic markers for cancer. They may also be useful as targets for immunotherapy with antibodies that recognize the tumor associated antigen, and thus selectively target tumor cells. Examples of tumor associated antigens include carcinoembryonic antigen (CEA), a glycoprotein expressed on gastrointestinal cancers and also present in many adenocarcinomas of endodermal origin; epithelial cell adhesion molecule (Ep-CAM), which is highly expressed by colorectal, pancreatic and non-small cell lung cancers, and is the target of the monoclonal antibody Edrecolomab; and Her2/neu, a member of the EGFR family that is overexpressed in approximately 25% of breast cancers as well as adenocarcinomas of the ovary, prostate, lung and gastrointestinal tract, and is the target of the humanized antibody Trastuzumab. For review, see, for example, Adams, G. P. and Weiner, L. M. (2005) Nature Biotechnol. 23(9): 1147-1157; and Schrama et al. (2006) Nature Reviews 5: 147-159.

C16orf54 is a single pass type I transmembrane protein composed of 224 amino acids. The protein comprises a 31 amino acid N-terminal extracellular domain, a single transmembrane domain, and a 171 amino acid C-terminal intracellular cytoplasmic domain. Orthologues of C16orf54 are found in other species, including primates, bovines, rat and mouse, but the C16orf54 amino acid sequence does not share significant sequence homology to any proteins of known function.

C16orf54 was initially identified in libraries from spleen tissue (European Patent Application No. EP1308459; International Patent Application No. WO2003/068943) as a predicted secreted or transmembrane protein. C16orf54 was also identified as a marker indicative of metastasis to bone tissue by comparison of expression levels in bone metastases of breast tumors as compared to lung, liver, brain and skin metastases. C16orf54 was overexpressed in bone metastases of breast tumors as compared to other metastases, and as compared to expression in normal bone (International Patent Application No. WO2008/104543). C16orf54 (referred to as A1467606) was identified as a transcription target of RUNX1/AML1 and is expressed during development of the hematopoietic system in vivo and its expression is detected in the CD41+ cell population. See Ferraras, C. et al. (2011) Blood 118: 594-597 and Supplement.

As disclosed herein, analysis of fresh chronic lymphocytic leukemia (CLL) tumor samples from patients using surface tagged antigen profiling (sTAg) of the cell surface proteome identified the transmembrane protein C16orf54 as being present at high density on the surface of CLL tumor cells. C16orf54 is therefore a target for the treatment of CLL, for example, by using binding agents such as antibodies which specifically bind to C16orf54.

The invention provides antibodies to C16orf54 that are useful in the diagnosis and treatment of various types of human cancers.

### SUMMARY

The invention provides antibodies that bind C16orf54, including antibody-drug conjugates comprising the antibodies, and methods of use of the antibodies and the antibody-drug conjugates, including for the diagnosis and treatment of cancers.

Using in-solution labeling of intact CLL tumor cell surfaces, followed by high-resolution, solution-based liquid chromatography coupled tandem mass spectrometry (LC-MS/MS), C16orf54 was identified as being present at high density on the surface of a majority of CLL cell subtypes as compared to normal cells including developing blood cells. Thus, the invention provides anti-C16orf54 antibodies and methods of using such antibodies in the treatment of CLL and other hematologic cancers, including but not limited to acute lymphocytic leukemia (ALL), acute myeloid leukemia (AML), chronic myeloid leukemia (CML), lymphoma, including Hodgkin and non-Hodgkin lymphoma, multiple myeloma, as well as solid tumors such as breast cancer and pancreatic cancer, and metastases of any of these cancers. In an embodiment, the invention provides an isolated antibody or a functional fragment thereof that specifically binds to the extracellular domain of C16orf54 (amino acids 1-32 of SEQ ID NO:1). In some embodiments, the antibody or functional fragment dissociates from the extracellular domain of C16orf54 with a K_{d} of 10⁻⁸ M or less as determined by biolayer interferometry. In some embodiments, the antibody or functional fragment dissociates from the extracellular domain of C16orf54 with a k_{off} rate constant of 1×10⁻³ s⁻¹ or less, as determined by biolayer interferometry. In some embodiments, the antibody or functional fragment dissociates from the extracellular domain of SEQ ID NO:1 with a K_{d} of 10⁻⁸ M or less and a k_{off} rate constant of 1×10⁻³ s⁻¹ or less, both determined by biolayer interferometry. In an embodiment, the invention provides an isolated antibody or a functional fragment thereof comprising all three heavy chain complementarity determining regions (CDRs) from a heavy chain variable domain having an amino acid sequence selected from the group consisting of SEQ ID NO: 4, SEQ ID NO: 8, SEQ ID NO:12, SEQ ID NO:16, SEQ ID NO:20, SEQ ID NO:24, SEQ ID NO:28, SEQ ID NO:32, SEQ ID NO:36, SEQ ID NO:40, SEQ ID NO:44, SEQ ID NO:48, SEQ ID NO:52, SEQ ID NO:56, SEQ ID NO:60, SEQ ID NO:64, SEQ ID NO:132, SEQ ID NO:134, SEQ ID NO:136, SEQ ID NO:138, SEQ ID NO:140, SEQ ID NO:142, SEQ ID NO:144, and SEQ ID NO:146, and/or all three light chain CDRs from a light chain variable domain having an amino acid sequence selected from the group consisting of SEQ ID NO: 6, SEQ ID NO: 10, and SEQ ID NO:14, SEQ ID NO:18, SEQ ID NO:22, SEQ ID NO:26, SEQ ID NO:30, SEQ ID NO:34, SEQ ID NO:38, SEQ ID NO:42, SEQ ID NO:46, SEQ ID NO:50, SEQ ID NO:54, SEQ ID NO:58, SEQ ID NO:62, and SEQ ID NO:66. In some embodiments, the antibody of a functional fragment thereof comprises all three heavy chain complementarity determining regions (CDRs) and/or all three light chain CDRs from: (a) the antibody designated R29-7-2A; (b) the antibody designated R29-7-1C; (c) the antibody designated R29-67-7A; (d) the antibody designated R29-8-136C; (e) the antibody designated R29-8-57B; (f) the antibody designated R29-7-54C; (g) the antibody designated R29-7-53A; (h) the antibody designated R29-8-50C; (i) the antibody designated R29-8-19B; (j) the antibody designated R29-8-58C; (k) the antibody designated R29-8-9B; (l) the antibody designated R29-8-28C; (m) the antibody designated R29-8-120B; (n) the antibody designated R29-8-75B; (o) the antibody designated R29-8-36C; (p) the antibody designated R29-8-12A; (q) the antibody designated R29-8-93B; (r) the antibody designated R29-8-51B; (s) the antibody designated R29-8-30A; (t) the antibody designated R29-8-18B; (u) the antibody designated R29-7-38C; (v) the antibody designated R29-7-49A; (w) the antibody designated R29-7-13A; or (x) the antibody designated R29-67-4A. In some embodiments, the antibody or functional fragment thereof comprises all three heavy chain CDRs or all three light chain CDRs from the antibody designated R29-7-2A. In some embodiments, the antibody or functional fragment thereof comprises all three heavy chain CDRs or all three light chain CDRs from the antibody designated R29-7-1C. In some embodiments, the antibody or functional fragment thereof comprises all three heavy chain CDRs or all three light chain CDRs from the antibody designated R29-67-7A. In some embodiments, the antibody or functional fragment thereof comprises all three heavy chain CDRs or all three light chain CDRs from the antibody designated R29-8-136C. In some embodiments, the antibody or functional fragment thereof comprises all three heavy chain CDRs or all three light chain CDRs from the antibody designated R29-8-57B. In some embodiments, the antibody or functional fragment thereof comprises all three heavy chain CDRs or all three light chain CDRs from the antibody designated R29-7-54C. In some embodiments, the antibody or functional fragment thereof comprises all three heavy chain CDRs or all three light chain CDRs from the antibody designated R29-7-53A. In some embodiments, the antibody or functional fragment thereof comprises all three heavy chain CDRs or all three light chain CDRs from the antibody designated R29-8-50C. In some embodiments, the antibody or functional fragment thereof comprises all three heavy chain CDRs or all three light chain CDRs from the antibody designated R29-8-19B. In some embodiments, the antibody or functional fragment thereof comprises all three heavy chain CDRs or all three light chain CDRs from the antibody designated R29-8-58C. In some embodiments, the antibody or functional fragment thereof comprises all three heavy chain CDRs or all three light chain CDRs from the antibody designated R29-8-9B. In some embodiments, the antibody or functional fragment thereof comprises all three heavy chain CDRs or all three light chain CDRs from the antibody designated R29-8-28C. In some embodiments, the antibody or functional fragment thereof comprises all three heavy chain CDRs or all three light chain CDRs from the antibody designated R29-8-120B. In some embodiments, the antibody or functional fragment thereof comprises all three heavy chain CDRs or all three light chain CDRs from the antibody designated R29-8-75B. In some embodiments, the antibody or functional fragment thereof comprises all three heavy chain CDRs or all three light chain CDRs from the antibody designated R29-8-36C. In some embodiments, the antibody or functional fragment thereof comprises all three heavy chain CDRs or all three light chain CDRs from the antibody designated R29-8-12A. In some embodiments, the antibody or functional fragment thereof comprises all three heavy chain CDRs or all three light chain CDRs from the antibody designated R29-8-93B. In some embodiments, the antibody or functional fragment thereof comprises all three heavy chain CDRs or all three light chain CDRs from the antibody designated R29-8-51B. In some embodiments, the antibody or functional fragment thereof comprises all three heavy chain CDRs or all three light chain CDRs from the antibody designated R29-8-30A. In some embodiments, the antibody or functional fragment thereof comprises all three heavy chain CDRs or all three light chain CDRs from the antibody designated R29-8-18B. In some embodiments, the antibody or functional fragment thereof comprises all three heavy chain CDRs or all three light chain CDRs from the antibody designated R29-7-38C. In some embodiments, the antibody or functional fragment thereof comprises all three heavy chain CDRs or all three light chain CDRs from the antibody designated R29-7-49A. In some embodiments, the antibody or functional fragment thereof comprises all three heavy chain CDRs or all three light chain CDRs from the antibody designated R29-7-13A. In some embodiments, the antibody or functional fragment thereof comprises all three heavy chain CDRs or all three light chain CDRs from the antibody designated R29-67-4A.

In an embodiment, the invention provides an isolated antibody or a functional fragment thereof comprising all three heavy chain CDRs from a heavy chain variable domain having an amino acid sequence selected from the group consisting of SEQ ID NO: 4, SEQ ID NO: 8, SEQ ID NO:12, SEQ ID NO:16, SEQ ID NO:20, SEQ ID NO:24, SEQ ID NO:28, SEQ ID NO:32, SEQ ID NO:36, SEQ ID NO:40, SEQ ID NO:44, SEQ ID NO:48, SEQ ID NO:52, SEQ ID NO:56, SEQ ID NO:60, SEQ ID NO:64, SEQ ID NO:132, SEQ ID NO:134, SEQ ID NO:136, SEQ ID NO:138, SEQ ID NO:140, SEQ ID NO:142, SEQ ID NO:144, and SEQ ID NO:146, and all three light chain CDRs from a light chain variable domain having an amino acid sequence selected from the group consisting of SEQ ID NO: 6, SEQ ID NO: 10, and SEQ ID NO:14, SEQ ID NO:18, SEQ ID NO:22, SEQ ID NO:26, SEQ ID NO:30, SEQ ID NO:34, SEQ ID NO:38, SEQ ID NO:42, SEQ ID NO:46, SEQ ID NO:50, SEQ ID NO:54, SEQ ID NO:58, SEQ ID NO:62, and SEQ ID NO:66. In some embodiments, the antibody or functional fragment thereof comprises all heavy and light chain complementarity determining regions (CDRs) from: (a) the antibody designated R29-7-2A; (b) the antibody designated R29-7-1C; (c) the antibody designated R29-67-7A; (d) the antibody designated R29-8-136C; (e) the antibody designated R29-8-57B; (f) the antibody designated R29-7-54C; (g) the antibody designated R29-7-53A; (h) the antibody designated R29-8-50C; (i) the antibody designated R29-8-19B; (j) the antibody designated R29-8-58C; (k) the antibody designated R29-8-9B; (l) the antibody designated R29-8-28C; (m) the antibody designated R29-8-120B; (n) the antibody designated R29-8-75B; (o) the antibody designated R29-8-36C; (p) the antibody designated R29-8-12A; (q) the antibody designated R29-8-93B; (r) the antibody designated R29-8-51B; (s) the antibody designated R29-8-30A; (t) the antibody designated R29-8-18B; (u) the antibody designated R29-7-38C; (v) the antibody designated R29-7-49A; (w) the antibody designated R29-7-13A; or (x) the antibody designated R29-67-4A. In some embodiments, the antibody or functional fragment thereof comprises all heavy and light chain CDRs from the antibody designated R29-7-2A. In some embodiments, the antibody or functional fragment thereof comprises all heavy and light chain CDRs from the antibody designated R29-7-1C. In some embodiments, the antibody or functional fragment thereof comprises all heavy and light chain CDRs from the antibody designated R29-67-7A. In some embodiments, the antibody or functional fragment thereof comprises all heavy and light chain CDRs from the antibody designated R29-8-136C. In some embodiments, the antibody or functional fragment thereof comprises all heavy and light chain CDRs from the antibody designated R29-8-57B. In some embodiments, the antibody or functional fragment thereof comprises all heavy and light chain CDRs from the antibody designated R29-7-54C. In some embodiments, the antibody or functional fragment thereof comprises all heavy and light chain CDRs from the antibody designated R29-7-53A. In some embodiments, the antibody or functional fragment thereof comprises all heavy and light chain CDRs from the antibody designated R29-8-50C. In some embodiments, the antibody or functional fragment thereof comprises all heavy and light chain CDRs from the antibody designated R29-8-19B. In some embodiments, the antibody or functional fragment thereof comprises all heavy and light chain CDRs from the antibody designated R29-8-58C. In some embodiments, the antibody or functional fragment thereof comprises all heavy and light chain CDRs from the antibody designated R29-8-9B. In some embodiments, the antibody or functional fragment thereof comprises all heavy and light chain CDRs from the antibody designated R29-8-28C. In some embodiments, the antibody or functional fragment thereof comprises all heavy and light chain CDRs from the antibody designated R29-8-120B. In some embodiments, the antibody or functional fragment thereof comprises all heavy and light chain CDRs from the antibody designated R29-8-75B. In some embodiments, the antibody or functional fragment thereof comprises all heavy and light chain CDRs from the antibody designated R29-8-36C. In some embodiments, the antibody or functional fragment thereof comprises all heavy and light chain CDRs from the antibody designated R29-8-12A.

In some embodiments, the antibody comprises a heavy chain variable domain sequence selected from the group consisting of SEQ ID NO: 4, SEQ ID NO: 8, SEQ ID NO:12, SEQ ID NO:16, SEQ ID NO:20, SEQ ID NO:24, SEQ ID NO:28, SEQ ID NO:32, SEQ ID NO:36, SEQ ID NO:40, SEQ ID NO:44, SEQ ID NO:48, SEQ ID NO:52, SEQ ID NO:56, SEQ ID NO:60, SEQ ID NO:64, SEQ ID NO:132, SEQ ID NO:134, SEQ ID NO:136, SEQ ID NO:138, SEQ ID NO:140, SEQ ID NO:142, SEQ ID NO:144, and SEQ ID NO:146. In some embodiments, the antibody comprises a light chain variable domain sequence selected from the group consisting of SEQ ID NO: 6, SEQ ID NO: 10, SEQ ID NO:14, SEQ ID NO:18, SEQ ID NO:22, SEQ ID NO:26, SEQ ID NO:30, SEQ ID NO:34, SEQ ID NO:38, SEQ ID NO:42, SEQ ID NO:46, SEQ ID NO:50, SEQ ID NO:54, SEQ ID NO:58, SEQ ID NO:62, and SEQ ID NO:66. In some embodiments, the antibody comprises a heavy chain variable domain sequence selected from the group consisting of SEQ ID NO: 4, SEQ ID NO: 8, SEQ ID NO:12, SEQ ID NO:16, SEQ ID NO:20, SEQ ID NO:24, SEQ ID NO:28, SEQ ID NO:32, SEQ ID NO:36, SEQ ID NO:40, SEQ ID NO:44, SEQ ID NO:48, SEQ ID NO:52, SEQ ID NO:56, SEQ ID NO:60, SEQ ID NO:64, SEQ ID NO:132, SEQ ID NO:134, SEQ ID NO:136, SEQ ID NO:138, SEQ ID NO:140, SEQ ID NO:142, SEQ ID NO:144, and SEQ ID NO:146, and further comprises a light chain variable domain sequence selected from the group consisting of SEQ ID NO: 6, SEQ ID NO: 10, SEQ ID NO:14, SEQ ID NO:18, SEQ ID NO:22, SEQ ID NO:26, SEQ ID NO:30, SEQ ID NO:34, SEQ ID NO:38, SEQ ID NO:42, SEQ ID NO:46, SEQ ID NO:50, SEQ ID NO:54, SEQ ID NO:58, SEQ ID NO:62, and SEQ ID NO:66.

In an embodiment, the antibody comprises the heavy chain variable domain sequence of SEQ ID NO: 4 and the light chain variable domain sequence of SEQ ID NO: 6. In an embodiment, the antibody comprises the heavy chain variable domain sequence of SEQ ID NO: 8 and the light chain variable domain sequence of SEQ ID NO: 10. In an embodiment, the antibody comprises the heavy chain variable domain sequence of SEQ ID NO: 12 and the light chain variable domain sequence of SEQ ID NO: 14. In an embodiment, the antibody comprises the antibody comprises the heavy chain variable domain sequence of SEQ ID NO:16 and the light chain variable domain sequence of SEQ ID NO:18. In an embodiment, the antibody comprises the heavy chain variable domain sequence of SEQ ID NO:20 and the light chain variable domain sequence of SEQ ID NO:22. In an embodiment, the antibody comprises the heavy chain variable domain sequence of SEQ ID NO24: and the light chain variable domain sequence of SEQ ID NO:26. In an embodiment, the antibody comprises the heavy chain variable domain sequence of SEQ ID NO:28 and the light chain variable domain sequence of SEQ ID NO:30. In an embodiment, the antibody comprises the heavy chain variable domain sequence of SEQ ID NO:32 and the light chain variable domain sequence of SEQ ID NO:34. In an embodiment, the antibody comprises the heavy chain variable domain sequence of SEQ ID NO:36 and the light chain variable domain sequence of SEQ ID NO:38. In an embodiment, the antibody comprises the heavy chain variable domain sequence of SEQ ID NO:40 and the light chain variable domain sequence of SEQ ID NO:42. In an embodiment, the antibody comprises the heavy chain variable domain sequence of SEQ ID NO:44 and the light chain variable domain sequence of SEQ ID NO:46. In an embodiment, the antibody comprises the heavy chain variable domain sequence of SEQ ID NO:48 and the light chain variable domain sequence of SEQ ID NO:50. In an embodiment, the antibody comprises the heavy chain variable domain sequence of SEQ ID NO:52 and the light chain variable domain sequence of SEQ ID NO:54. In an embodiment, the antibody comprises the heavy chain variable domain sequence of SEQ ID NO:56 and the light chain variable domain sequence of SEQ ID NO:58. In an embodiment, the antibody comprises the heavy chain variable domain sequence of SEQ ID NO:60 and the light chain variable domain sequence of SEQ ID NO:62. In an embodiment, the antibody comprises the heavy chain variable domain sequence of SEQ ID NO:64 and the light chain variable domain sequence of SEQ ID NO:66.

In some embodiments, the invention provides an isolated antibody or functional fragment thereof that binds to C16orf54, wherein the antibody comprises a heavy chain variable (V_{H}) region comprising one, two, three or more VH CDR amino acid sequences of Tables 1-5, and/or a light chain variable (V_{L}) region comprising one, two, three or more VL CDR amino acid sequences of Tables 1-5. Accordingly, in some embodiments, the invention provides an isolated antibody or functional fragment thereof that binds to C16orf54, wherein the antibody comprises: (a) a V_{H} region comprising: (1) a V_{H} CDR1 having an amino acid sequence selected from the group consisting of: (i) GFTGSX₁YA (SEQ ID NO:67) wherein X₁ is a naturally occurring amino acid, (ii) GFTFSRFG (SEQ ID NO:73), (iii) GYSITSX₁YA (SEQ ID NO:79) wherein X₁ is a naturally occurring amino acid, (iv) GFSLTDYX₁ (SEQ ID NO:85) wherein X₁ is a naturally occurring amino acid, and (v) GFSFNTHA (SEQ ID NO:91); (2) a V_{H} CDR2 having an amino acid sequence selected from the group consisting of: (i) ITGX₁GGX₂X₃ (SEQ ID NO:68) wherein X₁, X₂, and X₃ is a naturally occurring amino acid, (ii) ISSGSSTI (SEQ ID NO:74), (iii) IX₁YSGX₂X₃ (SEQ ID NO:80) wherein X₁, X₂, and X₃ is a naturally occurring amino acid, (iv) IWGGGX₁T (SEQ ID NO:86) wherein X₁ is a naturally occurring amino acid, and (v) IRSKSNNYAR (SEQ ID NO:92); and/or (3) a V_{H} CDR3 having an amino acid sequence selected from the group consisting of: (i) X₁RGWDENDX₂ (SEQ ID NO:69) wherein X₁ and X₂ is a naturally occurring amino acid, (ii) ARVDYDVALAY (SEQ ID NO:75), (iii) AREX₁YDX₂X₃X₄YX₅MDY (SEQ ID NO:81) wherein X₁, X₂, X₃, X₄, and X₅ is a naturally occurring amino acid, or alternatively ATTGTR (SEQ ID NO:121), (iv) AKHEEVSRFAX₁ (SEQ ID NO:87) wherein X₁ is a naturally occurring amino acid, and (v) VKQGDGGFAY (SEQ ID NO:93); and/or (b) a V_{L} region comprising: (1) a V_{L} CDR1 having an amino acid sequence selected from the group consisting of: (i) QSLX₁X₂SNGNTY (SEQ ID NO:70) wherein X₁ and X₂ is a naturally occurring amino acid, (ii) QSIVHRNGNTY (SEQ ID NO:76), (iii) QX₁LLYSX₂NQKNY (SEQ ID NO:82) wherein X₁ and X₂ is a naturally occurring amino acid, (iv) QSLVYSNGNSY (SEQ ID NO:88), and (v) QSLLYSSNQKNY (SEQ ID NO:94); (2) a V_{L} CDR2 having an amino acid sequence of: (i) KVS (SEQ ID NO:71), or (ii) WAS (SEQ ID NO:83); and/or (3) a V_{L} CDR3 having an amino acid sequence selected from the group consisting of: (i) SQX₁THVPWT (SEQ ID NO:72) wherein X₁ is a naturally occurring amino acid, (ii) FQGSQWT (SEQ ID NO:78), (iii) QQYYX₁YRT (SEQ ID NO:84) wherein X₁ is a naturally occurring amino acid, (iv) SQSTHIPLT (SEQ ID NO:90), and (v) QQYYSYPPT (SEQ ID NO:96).

In some embodiments, the antibody comprises a heavy chain variable (V_{H}) region comprising: (1) a V_{H} CDR1 having an amino acid sequence selected from the group consisting of: (i) GFTGSX₁YA (SEQ ID NO:67) wherein X₁ is a naturally occurring amino acid, (ii) GFTFSRFG (SEQ ID NO:73), (iii) GYSITSX₁YA (SEQ ID NO:79) wherein X₁ is a naturally occurring amino acid, (iv) GFSLTDYX₁ (SEQ ID NO:85) wherein X₁ is a naturally occurring amino acid, and (v) GFSFNTHA (SEQ ID NO:91); (2) a V_{H} CDR2 having an amino acid sequence selected from the group consisting of: (i) ITGX₁GGX₂X₃ (SEQ ID NO:68) wherein X₁, X₂, and X₃ is a naturally occurring amino acid, (ii) ISSGSSTI (SEQ ID NO:74), (iii) IX₁YSGX₂X₃ (SEQ ID NO:80) wherein X₁, X₂, and X₃ is a naturally occurring amino acid, (iv) IWGGGX₁T (SEQ ID NO:86) wherein X₁ is a naturally occurring amino acid, and (v) IRSKSNNYAR (SEQ ID NO:92); and (3) a V_{H} CDR3 having an amino acid sequence selected from the group consisting of: (i) X₁ RGWDENDX₂ (SEQ ID NO:69) wherein X₁ and X₂ is a naturally occurring amino acid, (ii) ARVDYDVALAY (SEQ ID NO:75), (iii) AREX₁YDX₂X₃X₄YX₅MDY (SEQ ID NO:81) wherein X₁, X₂, X₃, X₄, and X₅ is a naturally occurring amino acid, or alternatively ATTGTR (SEQ ID NO:121), (iv) AKHEEVSRFAX₁ (SEQ ID NO:87) wherein X₁ is a naturally occurring amino acid, and (v) VKQGDGGFAY (SEQ ID NO:93).

In some embodiments, the antibody comprises a light chain variable (V_{L}) region comprising: (1) a V_{L} CDR1 having an amino acid sequence selected from the group consisting of: (i) QSLX₁X₂SNGNTY (SEQ ID NO:70) wherein X₁ and X₂ is a naturally occurring amino acid, (ii)QSIVHRNGNTY (SEQ ID NO:76), (iii) QX₁LLYSX₂NQKNY (SEQ ID NO:82) wherein X₁ and X₂ is a naturally occurring amino acid, (iv) QSLVYSNGNSY (SEQ ID NO:88), and (v) QSLLYSSNQKNY (SEQ ID NO:94); (2) a V_{L} CDR2 having an amino acid sequence of: (i) KVS (SEQ ID NO:71), or (ii) WAS (SEQ ID NO:83); and (3) a V_{L} CDR3 having an amino acid sequence selected from the group consisting of: (i) SQX₁THVPWT (SEQ ID NO:72) wherein X₁ is a naturally occurring amino acid, (ii) FQGSQWT (SEQ ID NO:78), (iii) QQYYX₁YRT (SEQ ID NO:84) wherein X₁ is a naturally occurring amino acid, (iv) SQSTHIPLT (SEQ ID NO:90), and (v) QQYYSYPPT (SEQ ID NO:96).

In some embodiments, the invention provides an isolated antibody or functional fragment thereof that binds to C16orf54, wherein the antibody comprises a heavy chain variable (V_{H}) region comprising one, two, three or more VH CDR amino acid sequences of Table 1, and/or a light chain variable (V_{L}) region comprising one, two, three or more VL CDR amino acid sequences of Table 1. Accordingly, in some embodiments, the antibody comprises: (a) a heavy chain variable (V_{H}) region comprising: (1) a V_{H} CDR1 having an amino acid sequence of GFTGSX₁YA (SEQ ID NO:67) wherein X₁ is a naturally occurring amino acid; (2) a V_{H} CDR2 having an amino acid sequence of ITGX₁GGX₂X₃ (SEQ ID NO:68) wherein X₁, X₂, and X₃ is a naturally occurring amino acid; and (3) a V_{H} CDR3 having an amino acid sequence of X₁RGWDENDX₂ (SEQ ID NO:69) wherein X₁ and X₂ is a naturally occurring amino acid; and (b) a light chain variable (V_{L}) region comprising: (1) a V_{L} CDR1 having an amino acid sequence of QSLX₁X₂SNGNTY (SEQ ID NO:70) wherein X₁ and X₂ is a naturally occurring amino acid; (2) a V_{L} CDR2 having an amino acid sequence of KVS (SEQ ID NO:71); and (3) a V_{L} CDR3 having an amino acid sequence of SQX₁THVPWT (SEQ ID NO:72) wherein X₁ is a naturally occurring amino acid. In one embodiment, the antibody comprises: (a) a heavy chain variable (V_{H}) region comprising: (1) a V_{H} CDR1 having an amino acid sequence of GFTGSX₁YA (SEQ ID NO:67) wherein X₁ is S, N, I or T; (2) a V_{H} CDR2 having an amino acid sequence of ITGX₁GGX₂X₃ (SEQ ID NO:68) wherein X1 is G or S, wherein X2 is G, S, T or R, wherein X3 is T, N or S; and (3) a V_{H} CDR3 having an amino acid sequence of X₁RGWDENDX₂ (SEQ ID NO:69) wherein X1 is A, G or T, wherein X2 is Y or L; and (b) a light chain variable (V_{L}) region comprising: (1) a V_{L} CDR1 having an amino acid sequence of QSLX₁X₂SNGNTY (SEQ ID NO:70) wherein X1 is V or L, wherein X2 is F or Y; (2) a V_{L} CDR2 having an amino acid sequence of KVS (SEQ ID NO:71); and (3) a V_{L} CDR3 having an amino acid sequence of SQX₁THVPWT (SEQ ID NO:72) wherein X1 is S or T. In one embodiment, the antibody comprises: (a) a heavy chain variable (V_{H}) region comprising: (1) a V_{H} CDR1 having an amino acid sequence selected from the group consisting of SEQ ID NO:76, SEQ ID NO:89, SEQ ID NO:95, and SEQ ID NO:96; (2) a V_{H} CDR2 having an amino acid sequence selected from the group consisting of SEQ ID NO:97, SEQ ID NO:98, SEQ ID NO:99, SEQ ID NO:100, SEQ ID NO:101, and SEQ ID NO:102; and (3) a V_{H} CDR3 having an amino acid sequence selected from the group consisting of SEQ ID NO:103, SEQ ID NO:104, and SEQ ID NO:105; and (b) a light chain variable (V_{L}) region comprising: (1) a V_{L} CDR1 having an amino acid sequence selected from the group consisting of SEQ ID NO:106, SEQ ID NO:107, and SEQ ID NO:108; (2) a V_{L} CDR2 having an amino acid sequence of KVS (SEQ ID NO:71); and (3) a V_{L} CDR3 having an amino acid sequence of SEQ ID NO:109 or SEQ ID NO:110.

In some embodiments, the invention provides an isolated antibody or functional fragment thereof that binds to C16orf54, wherein the antibody comprises a heavy chain variable (V_{H}) region comprising one, two, or three VH CDR amino acid sequences of Table 2, and/or a light chain variable (V_{L}) region comprising one, two, or three VL CDR amino acid sequences of Table 2. Accordingly, in some embodiments, the antibody comprises: (a) a heavy chain variable (V_{H}) region comprising: (1) a V_{H} CDR1 having an amino acid sequence of GFTFSRFG (SEQ ID NO:73); (2) a V_{H} CDR2 having an amino acid sequence of ISSGSSTI (SEQ ID NO:74); and (3) a V_{H} CDR3 having an amino acid sequence of ARVDYDVALAY (SEQ ID NO:75); and (b) a light chain variable (V_{L}) region comprising: (1) a V_{L} CDR1 having an amino acid sequence of QSIVHRNGNTY (SEQ ID NO:76); (2) a V_{L} CDR2 having an amino acid sequence of KVS (SEQ ID NO:71); and (3) a V_{L} CDR3 having an amino acid sequence of FQGSQWT (SEQ ID NO:78).

In some embodiments, the invention provides an isolated antibody or functional fragment thereof that binds to C16orf54, wherein the antibody comprises a heavy chain variable (V_{H}) region comprising one, two, three or more VH CDR amino acid sequences of Table 3, and/or a light chain variable (V_{L}) region comprising one, two, three or more VL CDR amino acid sequences of Table 3. Accordingly, in some embodiments, the antibody comprises: (a) a heavy chain variable (V_{H}) region comprising: (1) a V_{H} CDR1 having an amino acid sequence of GYSITSX₁YA (SEQ ID NO:79) wherein X₁ is a naturally occurring amino acid; (2) a V_{H} CDR2 having an amino acid sequence of IX₁YSGX₂X₃ (SEQ ID NO:80) wherein X₁, X₂, and X₃ is a naturally occurring amino acid; and (3) a V_{H} CDR3 having an amino acid sequence of AREX₁YDX₂X₃X₄YX₅MDY (SEQ ID NO:81) wherein X₁, X₂, X₃, X₄, and X₅ is a naturally occurring amino acid, or alternatively ATTGTR (SEQ ID NO:121); and (b) a light chain variable (V_{L}) region comprising: (1) a V_{L} CDR1 having an amino acid sequence of QX₁LLYSX₂NQKNY (SEQ ID NO:82) wherein X₁ and X₂ is a naturally occurring amino acid; (2) a V_{L} CDR2 having an amino acid sequence of WAS (SEQ ID NO:83); and (3) a V_{L} CDR3 having an amino acid sequence of QQYYX₁YRT (SEQ ID NO:84) wherein X₁ is a naturally occurring amino acid. In one embodiment, the antibody comprises: (a) a heavy chain variable (V_{H}) region comprising: (1) a V_{H} CDR1 having an amino acid sequence of GYSITSX₁YA (SEQ ID NO:79) wherein X1 is D or V; (2) a V_{H} CDR2 having an amino acid sequence of IX₁YSGX₂X₃ (SEQ ID NO:80) wherein X1 is N or S, wherein X2 is S, R or I, wherein X3 is T, S, I; and (3) a V_{H} CDR3 having an amino acid sequence of AREX₁YDX₂X₃X₄YX₅MDY (SEQ ID NO:81) wherein X1 is R, K or N, wherein X2 is G, N or Y, wherein X3 is V, Y or E, wherein X4 is Y or F, X5 is G or A, or alternatively ATTGTR (SEQ ID NO:121); and (b) a light chain variable (V_{L}) region comprising: (1) a V_{L} CDR1 having an amino acid sequence of QX₁LLYSX₂NQKNY (SEQ ID NO:82) wherein X1 is S or N, wherein X2 is S or T; (2) a V_{L} CDR2 having an amino acid sequence of WAS (SEQ ID NO:83); and (3) a V_{L} CDR3 having an amino acid sequence of QQYYX₁YRT (SEQ ID NO:84) wherein X1 is S or I. In one embodiment, the antibody comprises: (a) a heavy chain variable (V_{H}) region comprising: (1) a V_{H} CDR1 having an amino acid sequence of SEQ ID NO:111 or SEQ ID NO:112; (2) a V_{H} CDR2 having an amino acid sequence selected from the group consisting of SEQ ID NO:113, SEQ ID NO:114, SEQ ID NO:115, SEQ ID NO:116, and SEQ ID NO:117; and (3) a V_{H} CDR3 having an amino acid sequence selected from the group consisting of SEQ ID NO:118, SEQ ID NO:119, SEQ ID NO:120, and SEQ ID NO:121; and (b) a light chain variable (V_{L}) region comprising: (1) a V_{L} CDR1 having an amino acid sequence selected from the group consisting of SEQ ID NO:94, SEQ ID NO:122, and SEQ ID NO:123; (2) a V_{L} CDR2 having an amino acid sequence of WAS (SEQ ID NO:83); and (3) a V_{L} CDR3 having an amino acid sequence of SEQ ID NO:124 or SEQ ID NO:125.

In some embodiments, the invention provides an isolated antibody or functional fragment thereof that binds to C16orf54, wherein the antibody comprises a heavy chain variable (V_{H}) region comprising one, two, three or more VH CDR amino acid sequences of Table 4, and/or a light chain variable (V_{L}) region comprising one, two, three or more VL CDR amino acid sequences of Table 4. Accordingly, in some embodiments, the antibody comprises: (a) a heavy chain variable (V_{H}) region comprising: (1) a V_{H} CDR1 having an amino acid sequence of GFSLTDYX₁ (SEQ ID NO:85) wherein X₁ is a naturally occurring amino acid; (2) a V_{H} CDR2 having an amino acid sequence of IWGGGX₁T (SEQ ID NO:86) wherein X₁ is a naturally occurring amino acid; and (3) a V_{H} CDR3 having an amino acid sequence of AKHEEVSRFAX₁ (SEQ ID NO:87) wherein X₁ is a naturally occurring amino acid; and (b) a light chain variable (V_{L}) region comprising: (1) a V_{L} CDR1 having an amino acid sequence of QSLVYSNGNSY (SEQ ID NO:88); (2) a V_{L} CDR2 having an amino acid sequence of KVS (SEQ ID NO:71); and (3) a V_{L} CDR3 having an amino acid sequence of SQSTHIPLT (SEQ ID NO:90). In one embodiment, the antibody comprises: (a) a heavy chain variable (V_{H}) region comprising: (1) a V_{H} CDR1 having an amino acid sequence of GFSLTDYX₁ (SEQ ID NO:85) wherein X1 is A or G; (2) a V_{H} CDR2 having an amino acid sequence of IWGGGX₁T (SEQ ID NO:86) wherein X1 is R or G; and (3) a V_{H} CDR3 having an amino acid sequence of AKHEEVSRFAX₁ (SEQ ID NO:87) wherein X1 is Y or H; and (b) a light chain variable (V_{L}) region comprising: (1) a V_{L} CDR1 having an amino acid sequence of QSLVYSNGNSY (SEQ ID NO:88); (2) a V_{L} CDR2 having an amino acid sequence of KVS (SEQ ID NO:71); and (3) a V_{L} CDR3 having an amino acid sequence of SQSTHIPLT (SEQ ID NO:90). In one embodiment, the antibody comprises: (a) a heavy chain variable (V_{H}) region comprising: (1) a V_{H} CDR1 having an amino acid sequence of SEQ ID NO:126 or SEQ ID NO:127; (2) a V_{H} CDR2 having an amino acid sequence of SEQ ID NO:128 or SEQ ID NO:129; and (3) a V_{H} CDR3 having an amino acid sequence of SEQ ID NO:130 or SEQ ID NO:77; and (b) a light chain variable (V_{L}) region comprising: (1) a V_{L} CDR1 having an amino acid sequence of QSLVYSNGNSY (SEQ ID NO:88); (2) a V_{L} CDR2 having an amino acid sequence of KVS (SEQ ID NO:71); and (3) a V_{L} CDR3 having an amino acid sequence of SQSTHIPLT (SEQ ID NO:90).

In some embodiments, the invention provides an isolated antibody or functional fragment thereof that binds to C16orf54, wherein the antibody comprises a heavy chain variable (V_{H}) region comprising one, two, or three VH CDR amino acid sequences of Table 5, and/or a light chain variable (V_{L}) region comprising one, two, or three VL CDR amino acid sequences of Table 5. Accordingly, in some embodiments, the antibody comprises: (a) a heavy chain variable (V_{H}) region comprising: (1) a V_{H} CDR1 having an amino acid sequence of GFSFNTHA (SEQ ID NO:91); (2) a V_{H} CDR2 having an amino acid sequence of IRSKSNNYAR (SEQ ID NO:92); and (3) a V_{H} CDR3 having an amino acid sequence of VKQGDGGFAY (SEQ ID NO:93); and (b) a light chain variable (V_{L}) region comprising: (1) a V_{L} CDR1 having an amino acid sequence of QSLLYSSNQKNY (SEQ ID NO:94); (2)a V_{L} CDR2 having an amino acid sequence of WAS (SEQ ID NO:83); and (3) a V_{L} CDR3 having an amino acid sequence of QQYYSYPPT (SEQ ID NO:96).

In some embodiments, the invention provides an isolated antibody or functional fragment thereof that binds to C16orf54, wherein the antibody comprises (a) a heavy chain variable (V_{H}) region comprising a V_{H} CDR1, a V_{H} CDR2, and a V_{H} CDR3 amino acid sequence depicted in Tables 6-29; and/or (b) a light chain variable (V_{L}) region comprising a V_{L} CDR1, a V_{L} CDR2, and a V_{L} CDR3 amino acid sequence depicted in Tables 6, 10, 12-22, 24, 25 and 29. 84. In some embodiments, the antibody comprises a heavy chain variable (V_{H}) region comprising a V_{H} CDR1, a V_{H} CDR2, and a V_{H} CDR3 amino acid sequence depicted in Tables 6-29. In some embodiments, the antibody comprises a light chain variable (V_{L}) region comprising a V_{L} CDR1, a V_{L} CDR2, and a V_{L} CDR3 amino acid sequence depicted in Tables 6, 10, 12-22, 24, 25 and 29.

In some embodiments, the invention provides an isolated antibody or functional fragment thereof that binds to C16orf54, wherein the antibody comprises a heavy chain variable (V_{H}) region comprising one, two, three or more VH CDR amino acid sequences of Table 6, and/or a light chain variable (V_{L}) region comprising one, two, three or more VL CDR amino acid sequences of Table 6. Accordingly, in some embodiments, the antibody comprises: (a) a heavy chain variable (V_{H}) region comprising: (1) a V_{H} CDR1 having an amino acid sequence of selected from the group consisting of SEQ ID NO:76, 147, 161, 166, and 172; (2) a V_{H} CDR2 having an amino acid sequence of selected from the group consisting of SEQ ID NO:97, 148, 162, 167, and 173; and (3) a V_{H} CDR3 having an amino acid sequence of selected from the group consisting of SEQ ID NO:103, 149, 163, and 168; and (b) a light chain variable (V_{L}) region comprising: (1) a V_{L} CDR1 having an amino acid sequence of selected from the group consisting of SEQ ID NO:106, 150, 164, and 169; (2) a V_{L} CDR2 having an amino acid sequence of selected from the group consisting of SEQ ID NO:71, 160, and 170; and (3) a V_{L} CDR3 having an amino acid sequence of selected from the group consisting of SEQ ID NO:109, 165, and 171. In one embodiment, the antibody comprises: (a) a heavy chain variable (V_{H}) region comprising: (1) a V_{H} CDR1 having the amino acid sequence of SEQ ID NO:76; (2) a V_{H} CDR2 having the amino acid sequence of SEQ ID NO:97; and (3) a V_{H} CDR3 having the amino acid sequence of SEQ ID NO:103; and (b) a light chain variable (V_{L}) region comprising: (1) a V_{L} CDR1 having the amino acid sequence of SEQ ID NO:106; (2) a V_{L} CDR2 having the amino acid sequence of SEQ ID NO:71; and (3) a V_{L} CDR3 having the amino acid sequence of SEQ ID NO:109. In one embodiment, the antibody comprises: (a) a heavy chain variable (V_{H}) region comprising: (1) a V_{H} CDR1 having the amino acid sequence of SEQ ID NO:147; (2) a V_{H} CDR2 having the amino acid sequence of SEQ ID NO:148; and (3) a V_{H} CDR3 having the amino acid sequence of SEQ ID NO:149; and (b) a light chain variable (V_{L}) region comprising: (1) a V_{L} CDR1 having the amino acid sequence of SEQ ID NO:150; (2) a V_{L} CDR2 having the amino acid sequence of SEQ ID NO:160; and (3) a V_{L} CDR3 having the amino acid sequence of SEQ ID NO:109. In one embodiment, the antibody comprises: (a) a heavy chain variable (V_{H}) region comprising: (1) a V_{H} CDR1 having the amino acid sequence of SEQ ID NO:161; (2) a V_{H} CDR2 having the amino acid sequence of SEQ ID NO:162; and (3) a V_{H} CDR3 having the amino acid sequence of SEQ ID NO:163; and (b) a light chain variable (V_{L}) region comprising: (1) a V_{L} CDR1 having the amino acid sequence of SEQ ID NO:164; (2) a V_{L} CDR2 having the amino acid sequence of SEQ ID NO:71; and (3) a V_{L} CDR3 having the amino acid sequence of SEQ ID NO:165. In one embodiment, the antibody comprises: (a) a heavy chain variable (V_{H}) region comprising: (1) a V_{H} CDR1 having the amino acid sequence of SEQ ID NO:166; (2)a V_{H} CDR2 having the amino acid sequence of SEQ ID NO:167; and (3) a V_{H} CDR3 having the amino acid sequence of SEQ ID NO:168; and (b) a light chain variable (V_{L}) region comprising: (1) a V_{L} CDR1 having the amino acid sequence of SEQ ID NO:169; (2) a V_{L} CDR2 having the amino acid sequence of SEQ ID NO:170; and (3) a V_{L} CDR3 having the amino acid sequence of SEQ ID NO:171. In one embodiment, the antibody comprises: (a) a heavy chain variable (V_{H}) region comprising: (1) a V_{H} CDR1 having the amino acid sequence of SEQ ID NO:172; (2) a V_{H} CDR2 having the amino acid sequence of SEQ ID NO:173; and (3) a V_{H} CDR3 having the amino acid sequence of SEQ ID NO:149; and (b) a light chain variable (V_{L}) region comprising: (1) a V_{L} CDR1 having the amino acid sequence of SEQ ID NO:150; (2) a V_{L} CDR2 having the amino acid sequence of SEQ ID NO:160; and (3) a V_{L} CDR3 having the amino acid sequence of SEQ ID NO:109.

In some embodiments, the invention provides an isolated antibody or functional fragment thereof that binds to C16orf54, wherein the antibody comprises a heavy chain variable (V_{H}) region comprising one, two, three or more VH CDR amino acid sequences of Table 7, and/or a light chain variable (V_{L}) region comprising one, two, three or more VL CDR amino acid sequences of Table 7. Accordingly, in some embodiments, the antibody comprises: (a) a heavy chain variable (V_{H}) region comprising: (1) a V_{H} CDR1 having an amino acid sequence of selected from the group consisting of SEQ ID NO:76, 147, 161, 166, and 172; (2) a V_{H} CDR2 having an amino acid sequence of selected from the group consisting of SEQ ID NO:97, 148, 162, 167, and 173; and (3) a V_{H} CDR3 having an amino acid sequence of selected from the group consisting of SEQ ID NO:103, 149, 163, and 168. In one embodiment, the antibody comprises: (a) a heavy chain variable (V_{H}) region comprising: (1) a V_{H} CDR1 having the amino acid sequence of SEQ ID NO:76; (2) a V_{H} CDR2 having the amino acid sequence of SEQ ID NO:97; and (3) a V_{H} CDR3 having the amino acid sequence of SEQ ID NO:103. In one embodiment, the antibody comprises: (a) a heavy chain variable (V_{H}) region comprising: (1) a V_{H} CDR1 having the amino acid sequence of SEQ ID NO:147; (2) a V_{H} CDR2 having the amino acid sequence of SEQ ID NO:148; and (3) a V_{H} CDR3 having the amino acid sequence of SEQ ID NO:149. In one embodiment, the antibody comprises: (a) a heavy chain variable (V_{H}) region comprising: (1) a V_{H} CDR1 having the amino acid sequence of SEQ ID NO:161; (2) a V_{H} CDR2 having the amino acid sequence of SEQ ID NO:162; and (3) a V_{H} CDR3 having the amino acid sequence of SEQ ID NO:163. In one embodiment, the antibody comprises: (a) a heavy chain variable (V_{H}) region comprising: (1) a V_{H} CDR1 having the amino acid sequence of SEQ ID NO:166; (2) a V_{H} CDR2 having the amino acid sequence of SEQ ID NO:167; and (3) a V_{H} CDR3 having the amino acid sequence of SEQ ID NO:168. In one embodiment, the antibody comprises: (a) a heavy chain variable (V_{H}) region comprising: (1) a V_{H} CDR1 having the amino acid sequence of SEQ ID NO:172; (2) a V_{H} CDR2 having the amino acid sequence of SEQ ID NO:173; and (3) a V_{H} CDR3 having the amino acid sequence of SEQ ID NO:149. In one embodiment, the antibody comprises: (a) a heavy chain variable (V_{H}) region comprising: (1) a V_{H} CDR1 having an amino acid sequence of selected from the group consisting of SEQ ID NO:76, 147, 161, 166, and 172; (2) a V_{H} CDR2 having an amino acid sequence of selected from the group consisting of SEQ ID NO:97, 148, 162, 167, and 173; and (3) a V_{H} CDR3 having an amino acid sequence of selected from the group consisting of SEQ ID NO:103, 149, 163, and 168.

In some embodiments, the invention provides an isolated antibody or functional fragment thereof that binds to C16orf54, wherein the antibody comprises a heavy chain variable (V_{H}) region comprising one, two, three or more VH CDR amino acid sequences of Table 8, and/or a light chain variable (V_{L}) region comprising one, two, three or more VL CDR amino acid sequences of Table 8. Accordingly, in some embodiments, the antibody comprises: (a) a heavy chain variable (V_{H}) region comprising: (1) a V_{H} CDR1 having the amino acid sequence of SEQ ID NO:76; (2) a V_{H} CDR2 having the amino acid sequence of SEQ ID NO:97; and (3) a V_{H} CDR3 having the amino acid sequence of SEQ ID NO:103. In one embodiment, the antibody comprises: (a) a heavy chain variable (V_{H}) region comprising: (1) a V_{H} CDR1 having the amino acid sequence of SEQ ID NO:147; (2) a V_{H} CDR2 having the amino acid sequence of SEQ ID NO:148; and (3) a V_{H} CDR3 having the amino acid sequence of SEQ ID NO:149. In one embodiment, the antibody comprises: (a) a heavy chain variable (V_{H}) region comprising: (1) a V_{H} CDR1 having the amino acid sequence of SEQ ID NO:161; (2) a V_{H} CDR2 having the amino acid sequence of SEQ ID NO:162; and (3) a V_{H} CDR3 having the amino acid sequence of SEQ ID NO:163. In one embodiment, the antibody comprises: (a) a heavy chain variable (V_{H}) region comprising: (1) a V_{H} CDR1 having the amino acid sequence of SEQ ID NO:166; (2) a V_{H} CDR2 having the amino acid sequence of SEQ ID NO:167; and (3) a V_{H} CDR3 having the amino acid sequence of SEQ ID NO:168. In one embodiment, the antibody comprises: (a) a heavy chain variable (V_{H}) region comprising: (1) a V_{H} CDR1 having the amino acid sequence of SEQ ID NO:172; (2) a V_{H} CDR2 having the amino acid sequence of SEQ ID NO:173; and (3) a V_{H} CDR3 having the amino acid sequence of SEQ ID NO:149.

In some embodiments, the invention provides an isolated antibody or functional fragment thereof that binds to C16orf54, wherein the antibody comprises a heavy chain variable (V_{H}) region comprising one, two, three or more VH CDR amino acid sequences of Table 9, and/or a light chain variable (V_{L}) region comprising one, two, three or more VL CDR amino acid sequences of Table 9. Accordingly, in some embodiments, the antibody comprises: (a) a heavy chain variable (V_{H}) region comprising: (1) a V_{H} CDR1 having an amino acid sequence of selected from the group consisting of SEQ ID NO:89, 174, 176, 177, and 179; (2) a V_{H} CDR2 having an amino acid sequence of selected from the group consisting of SEQ ID NO:98, 175, 162, 178, and 180; and (3) a V_{H} CDR3 having an amino acid sequence of selected from the group consisting of SEQ ID NO:103, 149, 163, and 168. In one embodiment, the antibody comprises: (a) a heavy chain variable (V_{H}) region comprising: (1) a V_{H} CDR1 having the amino acid sequence of SEQ ID NO:89; (2) a V_{H} CDR2 having the amino acid sequence of SEQ ID NO:98; and (3) a V_{H} CDR3 having the amino acid sequence of SEQ ID NO:103. In one embodiment, the antibody comprises: (a) a heavy chain variable (V_{H}) region comprising: (1) a V_{H} CDR1 having the amino acid sequence of SEQ ID NO:174; (2) a V_{H} CDR2 having the amino acid sequence of SEQ ID NO:175; and (3) a V_{H} CDR3 having the amino acid sequence of SEQ ID NO:149. In one embodiment, the antibody comprises: (a) a heavy chain variable (V_{H}) region comprising: (1) a V_{H} CDR1 having the amino acid sequence of SEQ ID NO:179; (2) a V_{H} CDR2 having the amino acid sequence of SEQ ID NO:162; and (3) a V_{H} CDR3 having the amino acid sequence of SEQ ID NO:163. In one embodiment, the antibody comprises: (a) a heavy chain variable (V_{H}) region comprising: (1) a V_{H} CDR1 having the amino acid sequence of SEQ ID NO:177; (2) a V_{H} CDR2 having the amino acid sequence of SEQ ID NO:178; and (3) a V_{H} CDR3 having the amino acid sequence of SEQ ID NO:168. In one embodiment, the antibody comprises: (a) a heavy chain variable (V_{H}) region comprising: (1) a V_{H} CDR1 having the amino acid sequence of SEQ ID NO:179; (2) a V_{H} CDR2 having the amino acid sequence of SEQ ID NO:180; and (3) a V_{H} CDR3 having the amino acid sequence of SEQ ID NO:149.

In some embodiments, the invention provides an isolated antibody or functional fragment thereof that binds to C16orf54, wherein the antibody comprises a heavy chain variable (V_{H}) region comprising one, two, three or more VH CDR amino acid sequences of Table 10, and/or a light chain variable (V_{L}) region comprising one, two, three or more VL CDR amino acid sequences of Table 10. Accordingly, in some embodiments, the antibody comprises: (a) a heavy chain variable (V_{H}) region comprising: (1) a V_{H} CDR1 having an amino acid sequence of selected from the group consisting of SEQ ID NO:95, 181, 184, 186, and 189; (2) a V_{H} CDR2 having an amino acid sequence of selected from the group consisting of SEQ ID NO:99, 182, 162, 187, and 190; and (3) a V_{H} CDR3 having an amino acid sequence of selected from the group consisting of SEQ ID NO:103, 149, 163, and 168; and (b) a light chain variable (V_{L}) region comprising: (1) a V_{L} CDR1 having an amino acid sequence of selected from the group consisting of SEQ ID NO:107, 183, 185, and 188; (2) a V_{L} CDR2 having an amino acid sequence of selected from the group consisting of SEQ ID NO:71, 160, and 170; and (3) a V_{L} CDR3 having an amino acid sequence of selected from the group consisting of SEQ ID NO:109, 165 and 171. In one embodiment, the antibody comprises: (a) a heavy chain variable (V_{H}) region comprising: (1) a V_{H} CDR1 having the amino acid sequence of SEQ ID NO:95; (2) a V_{H} CDR2 having the amino acid sequence of SEQ ID NO:99; and (3) a V_{H} CDR3 having the amino acid sequence of SEQ ID NO:103; and (b) a light chain variable (V_{L}) region comprising: (1) a V_{L} CDR1 having the amino acid sequence of SEQ ID NO:107; (2) a V_{L} CDR2 having the amino acid sequence of SEQ ID NO:71; and (3) a V_{L} CDR3 having the amino acid sequence of SEQ ID NO:109. In one embodiment, the antibody comprises: (a) a heavy chain variable (V_{H}) region comprising: (1) a V_{H} CDR1 having the amino acid sequence of SEQ ID NO:181; (2) a V_{H} CDR2 having the amino acid sequence of SEQ ID NO:182; and (3) a V_{H} CDR3 having the amino acid sequence of SEQ ID NO:149; and (b) a light chain variable (V_{L}) region comprising: (1) a V_{L} CDR1 having the amino acid sequence of SEQ ID NO:183; (2) a V_{L} CDR2 having the amino acid sequence of SEQ ID NO:160; and (3) a V_{L} CDR3 having the amino acid sequence of SEQ ID NO:109. In one embodiment, the antibody comprises: (a) a heavy chain variable (V_{H}) region comprising: (1) a V_{H} CDR1 having the amino acid sequence of SEQ ID NO:184; (2) a V_{H} CDR2 having the amino acid sequence of SEQ ID NO:162; and (3) a V_{H} CDR3 having the amino acid sequence of SEQ ID NO:163; and (b) a light chain variable (V_{L}) region comprising: (1) a V_{L} CDR1 having the amino acid sequence of SEQ ID NO:185; (2) a V_{L} CDR2 having the amino acid sequence of SEQ ID NO:71; and (3) a V_{L} CDR3 having the amino acid sequence of SEQ ID NO:165. In one embodiment, the antibody comprises: (a) a heavy chain variable (V_{H}) region comprising: (1) a V_{H} CDR1 having the amino acid sequence of SEQ ID NO:186; (2) a V_{H} CDR2 having the amino acid sequence of SEQ ID NO:187; and (3) a V_{H} CDR3 having the amino acid sequence of SEQ ID NO:168; and (b) a light chain variable (V_{L}) region comprising: (1) a V_{L} CDR1 having the amino acid sequence of SEQ ID NO:188; (2) a V_{L} CDR2 having the amino acid sequence of SEQ ID NO:170; and (3) a V_{L} CDR3 having the amino acid sequence of SEQ ID NO:171. In one embodiment, the antibody comprises: (a) a heavy chain variable (V_{H}) region comprising: (1) a V_{H} CDR1 having the amino acid sequence of SEQ ID NO:189; (2) a V_{H} CDR2 having the amino acid sequence of SEQ ID NO:190; and (3) a V_{H} CDR3 having the amino acid sequence of SEQ ID NO:149; and (b) a light chain variable (V_{L}) region comprising: (1) a V_{L} CDR1 having the amino acid sequence of SEQ ID NO:183; (2) a V_{L} CDR2 having the amino acid sequence of SEQ ID NO:160; and (3) a V_{L} CDR3 having the amino acid sequence of SEQ ID NO:109.

In some embodiments, the invention provides an isolated antibody or functional fragment thereof that binds to C16orf54, wherein the antibody comprises a heavy chain variable (V_{H}) region comprising one, two, three or more VH CDR amino acid sequences of Table 11, and/or a light chain variable (V_{L}) region comprising one, two, three or more VL CDR amino acid sequences of Table 11. Accordingly, in some embodiments, the antibody comprises: (a) a heavy chain variable (V_{H}) region comprising: (1) a V_{H} CDR1 having an amino acid sequence of selected from the group consisting of SEQ ID NO:96, 191, 193, 195, and 197; (2) a V_{H} CDR2 having an amino acid sequence of selected from the group consisting of SEQ ID NO:100, 192, 194, 196, and 198; and (3) a V_{H} CDR3 having an amino acid sequence of selected from the group consisting of SEQ ID NO:103, 149, 163, and 168. In one embodiment, the antibody comprises: (a) a heavy chain variable (V_{H}) region comprising: (1) a V_{H} CDR1 having the amino acid sequence of SEQ ID NO:96; (2) a V_{H} CDR2 having the amino acid sequence of SEQ ID NO:100; and (3) a V_{H} CDR3 having the amino acid sequence of SEQ ID NO:103. In one embodiment, the antibody comprises: (a) a heavy chain variable (V_{H}) region comprising: (1) a V_{H} CDR1 having the amino acid sequence of SEQ ID NO:191; (2) a V_{H} CDR2 having the amino acid sequence of SEQ ID NO:192; and (3) a V_{H} CDR3 having the amino acid sequence of SEQ ID NO:149. In one embodiment, the antibody comprises: (a) a heavy chain variable (V_{H}) region comprising: (1) a V_{H} CDR1 having the amino acid sequence of SEQ ID NO:193; (2) a V_{H} CDR2 having the amino acid sequence of SEQ ID NO:194; and (3) a V_{H} CDR3 having the amino acid sequence of SEQ ID NO:163. In one embodiment, the antibody comprises: (a) a heavy chain variable (V_{H}) region comprising: (1) a V_{H} CDR1 having the amino acid sequence of SEQ ID NO:195; (2) a V_{H} CDR2 having the amino acid sequence of SEQ ID NO:196; and (3) a V_{H} CDR3 having the amino acid sequence of SEQ ID NO:168. In one embodiment, the antibody comprises: (a) a heavy chain variable (V_{H}) region comprising: (1) a V_{H} CDR1 having the amino acid sequence of SEQ ID NO:197; (2) a V_{H} CDR2 having the amino acid sequence of SEQ ID NO:198; and (3) a V_{H} CDR3 having the amino acid sequence of SEQ ID NO:149.

In some embodiments, the invention provides an isolated antibody or functional fragment thereof that binds to C16orf54, wherein the antibody comprises a heavy chain variable (V_{H}) region comprising one, two, three or more VH CDR amino acid sequences of Table 12, and/or a light chain variable (V_{L}) region comprising one, two, three or more VL CDR amino acid sequences of Table 12. Accordingly, in some embodiments, the antibody comprises: (a) a heavy chain variable (V_{H}) region comprising: (1) a V_{H} CDR1 having an amino acid sequence of selected from the group consisting of SEQ ID NO:89, 199, 176, 202, and 206; (2) a V_{H} CDR2 having an amino acid sequence of selected from the group consisting of SEQ ID NO:101, 200, 194, 203, and 207; and (3) a V_{H} CDR3 having an amino acid sequence of selected from the group consisting of SEQ ID NO:104, 149, 163, and 204; and (b) a light chain variable (V_{L}) region comprising: (1) a V_{L} CDR1 having an amino acid sequence of selected from the group consisting of SEQ ID NO:107, 183, 185, and 188; (2) a V_{L} CDR2 having an amino acid sequence of selected from the group consisting of SEQ ID NO:71, 160 and 170; and (3) a V_{L} CDR3 having an amino acid sequence of selected from the group consisting of SEQ ID NO:110, 201, and 205. In one embodiment, the antibody comprises: (a) a heavy chain variable (V_{H}) region comprising: (1) a V_{H} CDR1 having the amino acid sequence of SEQ ID NO:89; (2) a V_{H} CDR2 having the amino acid sequence of SEQ ID NO:101; and (3) a V_{H} CDR3 having the amino acid sequence of SEQ ID NO:104; and (b) a light chain variable (V_{L}) region comprising: (1) a V_{L} CDR1 having the amino acid sequence of SEQ ID NO:107; (2) a V_{L} CDR2 having the amino acid sequence of SEQ ID NO:71; and (3) a V_{L} CDR3 having the amino acid sequence of SEQ ID NO:110. In one embodiment, the antibody comprises: (a) a heavy chain variable (V_{H}) region comprising: (1) a V_{H} CDR1 having the amino acid sequence of SEQ ID NO:199; (2) a V_{H} CDR2 having the amino acid sequence of SEQ ID NO:200; and (3) a V_{H} CDR3 having the amino acid sequence of SEQ ID NO:149; and (b) a light chain variable (V_{L}) region comprising: (1) a V_{L} CDR1 having the amino acid sequence of SEQ ID NO:183; (2) a V_{L} CDR2 having the amino acid sequence of SEQ ID NO:160; and (3) a V_{L} CDR3 having the amino acid sequence of SEQ ID NO:110. In one embodiment, the antibody comprises: (a) a heavy chain variable (V_{H}) region comprising: (1) a V_{H} CDR1 having the amino acid sequence of SEQ ID NO:176; (2) a V_{H} CDR2 having the amino acid sequence of SEQ ID NO:194; and (3) a V_{H} CDR3 having the amino acid sequence of SEQ ID NO:163; and (b) a light chain variable (V_{L}) region comprising: (1) a V_{L} CDR1 having the amino acid sequence of SEQ ID NO:185; (2) a V_{L} CDR2 having the amino acid sequence of SEQ ID NO:71; and (3) a V_{L} CDR3 having the amino acid sequence of SEQ ID NO:201. In one embodiment, the antibody comprises: (a) a heavy chain variable (V_{H}) region comprising: (1) a V_{H} CDR1 having the amino acid sequence of SEQ ID NO:202; (2) a V_{H} CDR2 having the amino acid sequence of SEQ ID NO:203; and (3) a V_{H} CDR3 having the amino acid sequence of SEQ ID NO:204; and (b) a light chain variable (V_{L}) region comprising: (1) a V_{L} CDR1 having the amino acid sequence of SEQ ID NO:188; (2) a V_{L} CDR2 having the amino acid sequence of SEQ ID NO:170; and (3) a V_{L} CDR3 having the amino acid sequence of SEQ ID NO:205. In one embodiment, the antibody comprises: (a) a heavy chain variable (V_{H}) region comprising: (1) a V_{H} CDR1 having the amino acid sequence of SEQ ID NO:206; (2) a V_{H} CDR2 having the amino acid sequence of SEQ ID NO:207; and (3) a V_{H} CDR3 having the amino acid sequence of SEQ ID NO:149; and (b) a light chain variable (V_{L}) region comprising: (1) a V_{L} CDR1 having the amino acid sequence of SEQ ID NO:183; (2) a V_{L} CDR2 having the amino acid sequence of SEQ ID NO:160; and (3) a V_{L} CDR3 having the amino acid sequence of SEQ ID NO:110. In one embodiment, the antibody comprises: (a) a heavy chain variable (V_{H}) region comprising: (1) a V_{H} CDR1 having an amino acid sequence of selected from the group consisting of SEQ ID NO:76, 208, 161, 211, and 213; (2) a V_{H} CDR2 having an amino acid sequence of selected from the group consisting of SEQ ID NO:101, 209, 194, 203, and 207; and (3) a V_{H} CDR3 having an amino acid sequence of selected from the group consisting of SEQ ID NO:105, 210, 163, and 212; and (b) a light chain variable (V_{L}) region comprising: (1) a V_{L} CDR1 having an amino acid sequence of selected from the group consisting of SEQ ID NO:107, 183, 185, and 188; (2) a V_{L} CDR2 having an amino acid sequence of selected from the group consisting of SEQ ID NO:71, 160, and 170; and (3) a V_{L} CDR3 having an amino acid sequence of selected from the group consisting of SEQ ID NO:110, 201, and 205.

In some embodiments, the invention provides an isolated antibody or functional fragment thereof that binds to C16orf54, wherein the antibody comprises a heavy chain variable (V_{H}) region comprising one, two, three or more VH CDR amino acid sequences of Table 13, and/or a light chain variable (V_{L}) region comprising one, two, three or more VL CDR amino acid sequences of Table 13. Accordingly, in some embodiments, the antibody comprises: (a) a heavy chain variable (V_{H}) region comprising: (1) a V_{H} CDR1 having the amino acid sequence of SEQ ID NO:76; (2) a V_{H} CDR2 having the amino acid sequence of SEQ ID NO:101; and (3) a V_{H} CDR3 having the amino acid sequence of SEQ ID NO:105; and (b) a light chain variable (V_{L}) region comprising: (1) a V_{L} CDR1 having the amino acid sequence of SEQ ID NO:107; (2) a V_{L} CDR2 having the amino acid sequence of SEQ ID NO:71; and (3) a V_{L} CDR3 having the amino acid sequence of SEQ ID NO:110. In one embodiment, the antibody comprises: (a) a heavy chain variable (V_{H}) region comprising: (1) a V_{H} CDR1 having the amino acid sequence of SEQ ID NO:208; (2) a V_{H} CDR2 having the amino acid sequence of SEQ ID NO:209; and (3) a V_{H} CDR3 having the amino acid sequence of SEQ ID NO:210; and (b) a light chain variable (V_{L}) region comprising: (1) a V_{L} CDR1 having the amino acid sequence of SEQ ID NO:183; (2) a V_{L} CDR2 having the amino acid sequence of SEQ ID NO:160; and (3) a V_{L} CDR3 having the amino acid sequence of SEQ ID NO:110. In one embodiment, the antibody comprises: (a) a heavy chain variable (V_{H}) region comprising: (1) a V_{H} CDR1 having the amino acid sequence of SEQ ID NO:161; (2) a V_{H} CDR2 having the amino acid sequence of SEQ ID NO:194; and (3) a V_{H} CDR3 having the amino acid sequence of SEQ ID NO:163; and (b) a light chain variable (V_{L}) region comprising: (1) a V_{L} CDR1 having the amino acid sequence of SEQ ID NO:185; (2) a V_{L} CDR2 having the amino acid sequence of SEQ ID NO:71; and (3) a V_{L} CDR3 having the amino acid sequence of SEQ ID NO:201. In one embodiment, the antibody comprises: (a) a heavy chain variable (V_{H}) region comprising: (1) a V_{H} CDR1 having the amino acid sequence of SEQ ID NO:211; (2) a V_{H} CDR2 having the amino acid sequence of SEQ ID NO:203; and (3) a V_{H} CDR3 having the amino acid sequence of SEQ ID NO:212; and (b) a light chain variable (V_{L}) region comprising: (1) a V_{L} CDR1 having the amino acid sequence of SEQ ID NO:188; (2) a V_{L} CDR2 having the amino acid sequence of SEQ ID NO:170; and (3) a V_{L} CDR3 having the amino acid sequence of SEQ ID NO:205. In one embodiment, the antibody comprises: (a) a heavy chain variable (V_{H}) region comprising: (1) a V_{H} CDR1 having the amino acid sequence of SEQ ID NO:213; (2) a V_{H} CDR2 having the amino acid sequence of SEQ ID NO:207; and (3) a V_{H} CDR3 having the amino acid sequence of SEQ ID NO:210; and (b) a light chain variable (V_{L}) region comprising: (1) a V_{L} CDR1 having the amino acid sequence of SEQ ID NO:183; (2) a V_{L} CDR2 having the amino acid sequence of SEQ ID NO:160; and (3) a V_{L} CDR3 having the amino acid sequence of SEQ ID NO:110.

In some embodiments, the invention provides an isolated antibody or functional fragment thereof that binds to C16orf54, wherein the antibody comprises a heavy chain variable (V_{H}) region comprising one, two, three or more VH CDR amino acid sequences of Table 14, and/or a light chain variable (V_{L}) region comprising one, two, three or more VL CDR amino acid sequences of Table 14. Accordingly, in some embodiments, the antibody comprises: (a) a heavy chain variable (V_{H}) region comprising: (1) a V_{H} CDR1 having an amino acid sequence of selected from the group consisting of SEQ ID NO:76, 208, 161, 211, and 213; (2) a V_{H} CDR2 having an amino acid sequence of selected from the group consisting of SEQ ID NO:101, 209, 194, 203, and 207; and (3) a V_{H} CDR3 having an amino acid sequence of selected from the group consisting of SEQ ID NO:105, 210, 163, and 212; and (b) a light chain variable (V_{L}) region comprising: (1) a V_{L} CDR1 having an amino acid sequence of selected from the group consisting of SEQ ID NO:107, 183, 185, and 188; (2) a V_{L} CDR2 having an amino acid sequence of selected from the group consisting of SEQ ID NO:71, 160, and 170; and (3) a V_{L} CDR3 having an amino acid sequence of selected from the group consisting of SEQ ID NO:110, 201, and 205. In one embodiment, the antibody comprises: (a) a heavy chain variable (V_{H}) region comprising: (1) a V_{H} CDR1 having the amino acid sequence of SEQ ID NO:76; (2) a V_{H} CDR2 having the amino acid sequence of SEQ ID NO:101; and (3) a V_{H} CDR3 having the amino acid sequence of SEQ ID NO:105; and (b) a light chain variable (V_{L}) region comprising: (1) a V_{L} CDR1 having the amino acid sequence of SEQ ID NO:107; (2) a V_{L} CDR2 having the amino acid sequence of SEQ ID NO:71; and (3) a V_{L} CDR3 having the amino acid sequence of SEQ ID NO:110. In one embodiment, the antibody comprises: (a) a heavy chain variable (V_{H}) region comprising: (1) a V_{H} CDR1 having the amino acid sequence of SEQ ID NO:208; (2) a V_{H} CDR2 having the amino acid sequence of SEQ ID NO:209; and (3) a V_{H} CDR3 having the amino acid sequence of SEQ ID NO:210; and (b) a light chain variable (V_{L}) region comprising: (1) a V_{L} CDR1 having the amino acid sequence of SEQ ID NO:183; (2) a V_{L} CDR2 having the amino acid sequence of SEQ ID NO:160; and (3) a V_{L} CDR3 having the amino acid sequence of SEQ ID NO:110. In one embodiment, the antibody comprises: (a) a heavy chain variable (V_{H}) region comprising: (1) a V_{H} CDR1 having the amino acid sequence of SEQ ID NO:161; (2) a V_{H} CDR2 having the amino acid sequence of SEQ ID NO:194; and (3) a V_{H} CDR3 having the amino acid sequence of SEQ ID NO:163; and (b) a light chain variable (V_{L}) region comprising: (1) a V_{L} CDR1 having the amino acid sequence of SEQ ID NO:185; (2) a V_{L} CDR2 having the amino acid sequence of SEQ ID NO:71; and (3) a V_{L} CDR3 having the amino acid sequence of SEQ ID NO:201. In one embodiment, the antibody comprises: (a) a heavy chain variable (V_{H}) region comprising: (1) a V_{H} CDR1 having the amino acid sequence of SEQ ID NO:211; (2) a V_{H} CDR2 having the amino acid sequence of SEQ ID NO:203; and (3) a V_{H} CDR3 having the amino acid sequence of SEQ ID NO:212; and (b) a light chain variable (V_{L}) region comprising: (1) a V_{L} CDR1 having the amino acid sequence of SEQ ID NO:188; (2) a V_{L} CDR2 having the amino acid sequence of SEQ ID NO:170; and (3) a V_{L} CDR3 having the amino acid sequence of SEQ ID NO:205. In one embodiment, the antibody comprises: (a) a heavy chain variable (V_{H}) region comprising: (1) a V_{H} CDR1 having the amino acid sequence of SEQ ID NO:213; (2) a V_{H} CDR2 having the amino acid sequence of SEQ ID NO:207; and (3) a V_{H} CDR3 having the amino acid sequence of SEQ ID NO:210; and (b) a light chain variable (V_{L}) region comprising: (1) a V_{L} CDR1 having the amino acid sequence of SEQ ID NO:183; (2) a V_{L} CDR2 having the amino acid sequence of SEQ ID NO:160; and (3) a V_{L} CDR3 having the amino acid sequence of SEQ ID NO:110.

In some embodiments, the invention provides an isolated antibody or functional fragment thereof that binds to C16orf54, wherein the antibody comprises a heavy chain variable (V_{H}) region comprising one, two, three or more VH CDR amino acid sequences of Table 15, and/or a light chain variable (V_{L}) region comprising one, two, three or more VL CDR amino acid sequences of Table 15. Accordingly, in some embodiments, the antibody comprises: (a) a heavy chain variable (V_{H}) region comprising: (1) a V_{H} CDR1 having an amino acid sequence of selected from the group consisting of SEQ ID NO:76, 208, 161, 211, and 213; (2) a V_{H} CDR2 having an amino acid sequence of selected from the group consisting of SEQ ID NO:101, 209, 194, 203, and 207; and (3) a V_{H} CDR3 having an amino acid sequence of selected from the group consisting of SEQ ID NO:105, 210, 163, and 212; and (b) a light chain variable (V_{L}) region comprising: (1) a V_{L} CDR1 having an amino acid sequence of selected from the group consisting of SEQ ID NO:107, 183, 185, and 188; (2) a V_{L} CDR2 having an amino acid sequence of selected from the group consisting of SEQ ID NO:71, 160 and 170; and (3) a V_{L} CDR3 having an amino acid sequence of selected from the group consisting of SEQ ID NO:110, 201 and 205. In one embodiment, the antibody comprises: (a) a heavy chain variable (V_{H}) region comprising: (1) a V_{H} CDR1 having the amino acid sequence of SEQ ID NO:76; (2) a V_{H} CDR2 having the amino acid sequence of SEQ ID NO:101; and (3) a V_{H} CDR3 having the amino acid sequence of SEQ ID NO:105; and (b) a light chain variable (V_{L}) region comprising: (1) a V_{L} CDR1 having the amino acid sequence of SEQ ID NO:107; (2) a V_{L} CDR2 having the amino acid sequence of SEQ ID NO:71; and (3) a V_{L} CDR3 having the amino acid sequence of SEQ ID NO:110. In one embodiment, the antibody comprises: (a) a heavy chain variable (V_{H}) region comprising: (1) a V_{H} CDR1 having the amino acid sequence of SEQ ID NO:208; (2) a V_{H} CDR2 having the amino acid sequence of SEQ ID NO:209; and (3) a V_{H} CDR3 having the amino acid sequence of SEQ ID NO:210; and (b) a light chain variable (V_{L}) region comprising: (1) a V_{L} CDR1 having the amino acid sequence of SEQ ID NO:183; (2) a V_{L} CDR2 having the amino acid sequence of SEQ ID NO:160; and (3) a V_{L} CDR3 having the amino acid sequence of SEQ ID NO:110. In one embodiment, the antibody comprises: (a) a heavy chain variable (V_{H}) region comprising: (1) a V_{H} CDR1 having the amino acid sequence of SEQ ID NO:161; (2) a V_{H} CDR2 having the amino acid sequence of SEQ ID NO:194; and (3) a V_{H} CDR3 having the amino acid sequence of SEQ ID NO:163; and (b) a light chain variable (V_{L}) region comprising: (1) a V_{L} CDR1 having the amino acid sequence of SEQ ID NO:185; (2) a V_{L} CDR2 having the amino acid sequence of SEQ ID NO:71; and (3) a V_{L} CDR3 having the amino acid sequence of SEQ ID NO:201. In one embodiment, the antibody comprises: (a) a heavy chain variable (V_{H}) region comprising: (1) a V_{H} CDR1 having the amino acid sequence of SEQ ID NO:211; (2) a V_{H} CDR2 having the amino acid sequence of SEQ ID NO:203; and (3) a V_{H} CDR3 having the amino acid sequence of SEQ ID NO:212; and (b) a light chain variable (V_{L}) region comprising: (1) a V_{L} CDR1 having the amino acid sequence of SEQ ID NO:188; (2) a V_{L} CDR2 having the amino acid sequence of SEQ ID NO:170; and (3) a V_{L} CDR3 having the amino acid sequence of SEQ ID NO:205. In one embodiment, the antibody comprises: (a) a heavy chain variable (V_{H}) region comprising: (1) a V_{H} CDR1 having the amino acid sequence of SEQ ID NO:213; (2) a V_{H} CDR2 having the amino acid sequence of SEQ ID NO:207; and (3) a V_{H} CDR3 having the amino acid sequence of SEQ ID NO:210; and (b) a light chain variable (V_{L}) region comprising: (1) a V_{L} CDR1 having the amino acid sequence of SEQ ID NO:183; (2) a V_{L} CDR2 having the amino acid sequence of SEQ ID NO:160; and (3) a V_{L} CDR3 having the amino acid sequence of SEQ ID NO:110.

In some embodiments, the invention provides an isolated antibody or functional fragment thereof that binds to C16orf54, wherein the antibody comprises a heavy chain variable (V_{H}) region comprising one, two, three or more VH CDR amino acid sequences of Table 16, and/or a light chain variable (V_{L}) region comprising one, two, three or more VL CDR amino acid sequences of Table 16. Accordingly, in some embodiments, the antibody comprises: (a) a heavy chain variable (V_{H}) region comprising: (1) a V_{H} CDR1 having an amino acid sequence of selected from the group consisting of SEQ ID NO:76, 208, 161, 211, and 213; (2) a V_{H} CDR2 having an amino acid sequence of selected from the group consisting of SEQ ID NO:102, 214, 194, 164, and 218; and (3) a V_{H} CDR3 having an amino acid sequence of selected from the group consisting of SEQ ID NO:105, 210, 163, and 212; and (b) a light chain variable (V_{L}) region comprising: (1) a V_{L} CDR1 having an amino acid sequence of selected from the group consisting of SEQ ID NO:108, 215, 216, and 217; (2) a V_{L} CDR2 having an amino acid sequence of selected from the group consisting of SEQ ID NO:71, 160, and 170; and (3) a V_{L} CDR3 having an amino acid sequence of selected from the group consisting of SEQ ID NO:110, 201, and 205. In one embodiment, the antibody comprises: (a) a heavy chain variable (V_{H}) region comprising: (1) a V_{H} CDR1 having the amino acid sequence of SEQ ID NO:76; (2) a V_{H} CDR2 having the amino acid sequence of SEQ ID NO:102; and (3) a V_{H} CDR3 having the amino acid sequence of SEQ ID NO:105; and (b) a light chain variable (V_{L}) region comprising: (1) a V_{L} CDR1 having the amino acid sequence of SEQ ID NO:108; (2) a V_{L} CDR2 having the amino acid sequence of SEQ ID NO:71; and (3) a V_{L} CDR3 having the amino acid sequence of SEQ ID NO:110. In one embodiment, the antibody comprises: (a) a heavy chain variable (V_{H}) region comprising: (1) a V_{H} CDR1 having the amino acid sequence of SEQ ID NO:208; (2) a V_{H} CDR2 having the amino acid sequence of SEQ ID NO:214; and (3) a V_{H} CDR3 having the amino acid sequence of SEQ ID NO:210; and (b) a light chain variable (V_{L}) region comprising: (1) a V_{L} CDR1 having the amino acid sequence of SEQ ID NO:215; (2) a V_{L} CDR2 having the amino acid sequence of SEQ ID NO:160; and (3) a V_{L} CDR3 having the amino acid sequence of SEQ ID NO:110. In one embodiment, the antibody comprises: (a) a heavy chain variable (V_{H}) region comprising: (1) a V_{H} CDR1 having the amino acid sequence of SEQ ID NO:161; (2) a V_{H} CDR2 having the amino acid sequence of SEQ ID NO:194; and (3) a V_{H} CDR3 having the amino acid sequence of SEQ ID NO:163; and (b) a light chain variable (V_{L}) region comprising: (1) a V_{L} CDR1 having the amino acid sequence of SEQ ID NO:216; (2) a V_{L} CDR2 having the amino acid sequence of SEQ ID NO:71; and (3) a V_{L} CDR3 having the amino acid sequence of SEQ ID NO:201. In one embodiment, the antibody comprises: (a) a heavy chain variable (V_{H}) region comprising: (1) a V_{H} CDR1 having the amino acid sequence of SEQ ID NO:211; (2) a V_{H} CDR2 having the amino acid sequence of SEQ ID NO:164; and (3) a V_{H} CDR3 having the amino acid sequence of SEQ ID NO:212; and (b) a light chain variable (V_{L}) region comprising: (1) a V_{L} CDR1 having the amino acid sequence of SEQ ID NO:217; (2) a V_{L} CDR2 having the amino acid sequence of SEQ ID NO:170; and (3) a V_{L} CDR3 having the amino acid sequence of SEQ ID NO:205. In one embodiment, the antibody comprises: (a) a heavy chain variable (V_{H}) region comprising: (1) a V_{H} CDR1 having the amino acid sequence of SEQ ID NO:213; (2) a V_{H} CDR2 having the amino acid sequence of SEQ ID NO:218; and (3) a V_{H} CDR3 having the amino acid sequence of SEQ ID NO:210; and (b) a light chain variable (V_{L}) region comprising: (1) a V_{L} CDR1 having the amino acid sequence of SEQ ID NO:215; (2) a V_{L} CDR2 having the amino acid sequence of SEQ ID NO:160; and (3) a V_{L} CDR3 having the amino acid sequence of SEQ ID NO:110.

In some embodiments, the invention provides an isolated antibody or functional fragment thereof that binds to C16orf54, wherein the antibody comprises a heavy chain variable (V_{H}) region comprising one, two, three or more VH CDR amino acid sequences of Table 17, and/or a light chain variable (V_{L}) region comprising one, two, three or more VL CDR amino acid sequences of Table 17. Accordingly, in some embodiments, the antibody comprises: (a) a heavy chain variable (V_{H}) region comprising: (1) a V_{H} CDR1 having an amino acid sequence of selected from the group consisting of SEQ ID NO:76, 208, 161, 211, and 213; (2) a V_{H} CDR2 having an amino acid sequence of selected from the group consisting of SEQ ID NO:102, 214, 194, 164, and 218; and (3) a V_{H} CDR3 having an amino acid sequence of selected from the group consisting of SEQ ID NO:105, 210, 163, and 212; and (b) a light chain variable (V_{L}) region comprising: (1) a V_{L} CDR1 having an amino acid sequence of selected from the group consisting of SEQ ID NO:108, 215, 216, and 217; (2) a V_{L} CDR2 having an amino acid sequence of selected from the group consisting of SEQ ID NO:71, 160, and 170; and (3) a V_{L} CDR3 having an amino acid sequence of selected from the group consisting of SEQ ID NO:110, 201, and 205. In one embodiment, the antibody comprises: (a) a heavy chain variable (V_{H}) region comprising: (1) a V_{H} CDR1 having the amino acid sequence of SEQ ID NO:76; (2) a V_{H} CDR2 having the amino acid sequence of SEQ ID NO:102; and (3) a V_{H} CDR3 having the amino acid sequence of SEQ ID NO:105; and (b) a light chain variable (V_{L}) region comprising: (1) a V_{L} CDR1 having the amino acid sequence of SEQ ID NO:108; (2) a V_{L} CDR2 having the amino acid sequence of SEQ ID NO:71; and (3) a V_{L} CDR3 having the amino acid sequence of SEQ ID NO:110. In one embodiment, the antibody comprises: (a) a heavy chain variable (V_{H}) region comprising: (1) a V_{H} CDR1 having the amino acid sequence of SEQ ID NO:208; (2) a V_{H} CDR2 having the amino acid sequence of SEQ ID NO:214; and (3) a V_{H} CDR3 having the amino acid sequence of SEQ ID NO:210; and (b) a light chain variable (V_{L}) region comprising: (1) a V_{L} CDR1 having the amino acid sequence of SEQ ID NO:215; (2) a V_{L} CDR2 having the amino acid sequence of SEQ ID NO:160; and (3) a V_{L} CDR3 having the amino acid sequence of SEQ ID NO:110. In one embodiment, the antibody comprises: (a) a heavy chain variable (V_{H}) region comprising: (1) a V_{H} CDR1 having the amino acid sequence of SEQ ID NO:161; (2) a V_{H} CDR2 having the amino acid sequence of SEQ ID NO:194; and (3) a V_{H} CDR3 having the amino acid sequence of SEQ ID NO:163; and (b) a light chain variable (V_{L}) region comprising: (1) a V_{L} CDR1 having the amino acid sequence of SEQ ID NO:216; (2) a V_{L} CDR2 having the amino acid sequence of SEQ ID NO:71; and (3) a V_{L} CDR3 having the amino acid sequence of SEQ ID NO:201. In one embodiment, the antibody comprises: (a) a heavy chain variable (V_{H}) region comprising: (1) a V_{H} CDR1 having the amino acid sequence of SEQ ID NO:211; (2) a V_{H} CDR2 having the amino acid sequence of SEQ ID NO:164; and (3) a V_{H} CDR3 having the amino acid sequence of SEQ ID NO:212; and (b) a light chain variable (V_{L}) region comprising: (1) a V_{L} CDR1 having the amino acid sequence of SEQ ID NO:217; (2) a V_{L} CDR2 having the amino acid sequence of SEQ ID NO:170; and (3) a V_{L} CDR3 having the amino acid sequence of SEQ ID NO:205. In one embodiment, the antibody comprises: (a) a heavy chain variable (V_{H}) region comprising: (1) a V_{H} CDR1 having the amino acid sequence of SEQ ID NO:213; (2) a V_{H} CDR2 having the amino acid sequence of SEQ ID NO:218; and (3) a V_{H} CDR3 having the amino acid sequence of SEQ ID NO:210; and (b) a light chain variable (V_{L}) region comprising: (1) a V_{L} CDR1 having the amino acid sequence of SEQ ID NO:215; (2) a V_{L} CDR2 having the amino acid sequence of SEQ ID NO:160; and (3) a V_{L} CDR3 having the amino acid sequence of SEQ ID NO:110. In one embodiment, the antibody comprises: (a) a heavy chain variable (V_{H}) region comprising: (1) a V_{H} CDR1 having an amino acid sequence of selected from the group consisting of SEQ ID NO:73, 219, 224, 229, and 318; (2) a V_{H} CDR2 having an amino acid sequence of selected from the group consisting of SEQ ID NO:74, 220, 225, 230, and 319; and (3) a V_{H} CDR3 having an amino acid sequence of selected from the group consisting of SEQ ID NO: 75, 221, 226, and 231; and (b) a light chain variable (V_{L}) region comprising: (1) a V_{L} CDR1 having an amino acid sequence of selected from the group consisting of SEQ ID NO:76, 222, 227, and 232; (2) a V_{L} CDR2 having an amino acid sequence of selected from the group consisting of SEQ ID NO:71, 160, and 170; and (3) a V_{L} CDR3 having an amino acid sequence of selected from the group consisting of SEQ ID NO:78, 228, and 233.

In some embodiments, the invention provides an isolated antibody or functional fragment thereof that binds to C16orf54, wherein the antibody comprises a heavy chain variable (V_{H}) region comprising one, two, three or more VH CDR amino acid sequences of Table 18, and/or a light chain variable (V_{L}) region comprising one, two, three or more VL CDR amino acid sequences of Table 18. Accordingly, in some embodiments, the antibody comprises: (a) a heavy chain variable (V_{H}) region comprising: (1) a V_{H} CDR1 having the amino acid sequence of SEQ ID NO:73; (2) a V_{H} CDR2 having the amino acid sequence of SEQ ID NO:74; and (3) a V_{H} CDR3 having the amino acid sequence of SEQ ID NO:75; and (b) a light chain variable (V_{L}) region comprising: (1) a V_{L} CDR1 having the amino acid sequence of SEQ ID NO:76; (2) a V_{L} CDR2 having the amino acid sequence of SEQ ID NO:71; and (3) a V_{L} CDR3 having the amino acid sequence of SEQ ID NO:78. In one embodiment, the antibody comprises: (a) a heavy chain variable (V_{H}) region comprising: (1) a V_{H} CDR1 having the amino acid sequence of SEQ ID NO:219; (2) a V_{H} CDR2 having the amino acid sequence of SEQ ID NO:220; and (3) a V_{H} CDR3 having the amino acid sequence of SEQ ID NO:221; and (b) a light chain variable (V_{L}) region comprising: (1) a V_{L} CDR1 having the amino acid sequence of SEQ ID NO:222; (2) a V_{L} CDR2 having the amino acid sequence of SEQ ID NO:160; and (3) a V_{L} CDR3 having the amino acid sequence of SEQ ID NO:78. In one embodiment, the antibody comprises: (a) a heavy chain variable (V_{H}) region comprising: (1) a V_{H} CDR1 having the amino acid sequence of SEQ ID NO:224; (2) a V_{H} CDR2 having the amino acid sequence of SEQ ID NO:225; and (3) a V_{H} CDR3 having the amino acid sequence of SEQ ID NO:226; and (b) a light chain variable (V_{L}) region comprising: (1) a V_{L} CDR1 having the amino acid sequence of SEQ ID NO:227; (2) a V_{L} CDR2 having the amino acid sequence of SEQ ID NO:71; and (3) a V_{L} CDR3 having the amino acid sequence of SEQ ID NO:228. In one embodiment, the antibody comprises: (a) a heavy chain variable (V_{H}) region comprising: (1) a V_{H} CDR1 having the amino acid sequence of SEQ ID NO:229; (2) a V_{H} CDR2 having the amino acid sequence of SEQ ID NO:230; and (3) a V_{H} CDR3 having the amino acid sequence of SEQ ID NO:231; and (b) a light chain variable (V_{L}) region comprising: (1) a V_{L} CDR1 having the amino acid sequence of SEQ ID NO:232; (2) a V_{L} CDR2 having the amino acid sequence of SEQ ID NO:170; and (3) a V_{L} CDR3 having the amino acid sequence of SEQ ID NO:233. In one embodiment, the antibody comprises: (a) a heavy chain variable (V_{H}) region comprising: (1) a V_{H} CDR1 having the amino acid sequence of SEQ ID NO:318; (2) a V_{H} CDR2 having the amino acid sequence of SEQ ID NO:319; and (3) a V_{H} CDR3 having the amino acid sequence of SEQ ID NO:221; and (b) a light chain variable (V_{L}) region comprising: (1) a V_{L} CDR1 having the amino acid sequence of SEQ ID NO:222; (2) a V_{L} CDR2 having the amino acid sequence of SEQ ID NO:160; and (3) a V_{L} CDR3 having the amino acid sequence of SEQ ID NO:78.

In some embodiments, the invention provides an isolated antibody or functional fragment thereof that binds to C16orf54, wherein the antibody comprises a heavy chain variable (V_{H}) region comprising one, two, three or more VH CDR amino acid sequences of Table 19, and/or a light chain variable (V_{L}) region comprising one, two, three or more VL CDR amino acid sequences of Table 19. Accordingly, in some embodiments, the antibody comprises: (a) a heavy chain variable (V_{H}) region comprising: (1) a V_{H} CDR1 having an amino acid sequence of selected from the group consisting of SEQ ID NO:111, 234, 240, 244, and 250; (2) a V_{H} CDR2 having an amino acid sequence of selected from the group consisting of SEQ ID NO:113, 235, 239, 245, and 251; and (3) a V_{H} CDR3 having an amino acid sequence of selected from the group consisting of SEQ ID NO:118, 236, 241, and 246; and (b) a light chain variable (V_{L}) region comprising: (1) a V_{L} CDR1 having an amino acid sequence of selected from the group consisting of SEQ ID NO:94, 237, 242, and 247; (2) a V_{L} CDR2 having an amino acid sequence of selected from the group consisting of SEQ ID NO:83, 238, and 248; and (3) a V_{L} CDR3 having an amino acid sequence of selected from the group consisting of SEQ ID NO:124, 243, and 249. In one embodiment, the antibody comprises: (a) a heavy chain variable (V_{H}) region comprising: (1) a V_{H} CDR1 having the amino acid sequence of SEQ ID NO:111; (2) a V_{H} CDR2 having the amino acid sequence of SEQ ID NO:113; and (3) a V_{H} CDR3 having the amino acid sequence of SEQ ID NO:118; and (b) a light chain variable (V_{L}) region comprising: (1) a V_{L} CDR1 having the amino acid sequence of SEQ ID NO:94; (2) a V_{L} CDR2 having the amino acid sequence of SEQ ID NO:83; and (3) a V_{L} CDR3 having the amino acid sequence of SEQ ID NO:124. In one embodiment, the antibody comprises: (a) a heavy chain variable (V_{H}) region comprising: (1) a V_{H} CDR1 having the amino acid sequence of SEQ ID NO:234; (2) a V_{H} CDR2 having the amino acid sequence of SEQ ID NO:235; and (3) a V_{H} CDR3 having the amino acid sequence of SEQ ID NO:236; and (b) a light chain variable (V_{L}) region comprising: (1) a V_{L} CDR1 having the amino acid sequence of SEQ ID NO:237; (2) a V_{L} CDR2 having the amino acid sequence of SEQ ID NO:238; and (3) a V_{L} CDR3 having the amino acid sequence of SEQ ID NO:124. In one embodiment, the antibody comprises: (a) a heavy chain variable (V_{H}) region comprising: (1) a V_{H} CDR1 having the amino acid sequence of SEQ ID NO:240; (2) a V_{H} CDR2 having the amino acid sequence of SEQ ID NO:239; and (3) a V_{H} CDR3 having the amino acid sequence of SEQ ID NO:241; and (b) a light chain variable (V_{L}) region comprising: (1) a V_{L} CDR1 having the amino acid sequence of SEQ ID NO:242; (2) a V_{L} CDR2 having the amino acid sequence of SEQ ID NO:83; and (3) a V_{L} CDR3 having the amino acid sequence of SEQ ID NO:243. In one embodiment, the antibody comprises: (a) a heavy chain variable (V_{H}) region comprising: (1) a V_{H} CDR1 having the amino acid sequence of SEQ ID NO:244; (2) a V_{H} CDR2 having the amino acid sequence of SEQ ID NO:245; and (3) a V_{H} CDR3 having the amino acid sequence of SEQ ID NO:246; and (b) a light chain variable (V_{L}) region comprising: (1) a V_{L} CDR1 having the amino acid sequence of SEQ ID NO:247; (2) a V_{L} CDR2 having the amino acid sequence of SEQ ID NO:248; and (3) a V_{L} CDR3 having the amino acid sequence of SEQ ID NO:249. In one embodiment, the antibody comprises: (a) a heavy chain variable (V_{H}) region comprising: (1) a V_{H} CDR1 having the amino acid sequence of SEQ ID NO:250; (2) a V_{H} CDR2 having the amino acid sequence of SEQ ID NO:251; and (3) a V_{H} CDR3 having the amino acid sequence of SEQ ID NO:236; and (b) a light chain variable (V_{L}) region comprising: (1) a V_{L} CDR1 having the amino acid sequence of SEQ ID NO:237; (2) a V_{L} CDR2 having the amino acid sequence of SEQ ID NO:238; and (3) a V_{L} CDR3 having the amino acid sequence of SEQ ID NO:124.

In some embodiments, the invention provides an isolated antibody or functional fragment thereof that binds to C16orf54, wherein the antibody comprises a heavy chain variable (V_{H}) region comprising one, two, three or more VH CDR amino acid sequences of Table 20, and/or a light chain variable (V_{L}) region comprising one, two, three or more VL CDR amino acid sequences of Table 20. Accordingly, in some embodiments, the antibody comprises: (a) a heavy chain variable (V_{H}) region comprising: (1) a V_{H} CDR1 having an amino acid sequence of selected from the group consisting of SEQ ID NO:111, 234, 240, 244, and 250; (2) a V_{H} CDR2 having an amino acid sequence of selected from the group consisting of SEQ ID NO:114, 223, 239, 252, and 253; and (3) a V_{H} CDR3 having an amino acid sequence of selected from the group consisting of SEQ ID NO:118, 236, 241, and 246; and (b) a light chain variable (V_{L}) region comprising: (1) a V_{L} CDR1 having an amino acid sequence of selected from the group consisting of SEQ ID NO:94, 237, 242, and 247; (2) a V_{L} CDR2 having an amino acid sequence of selected from the group consisting of SEQ ID NO:83, 238, and 248; and (3) a V_{L} CDR3 having an amino acid sequence of selected from the group consisting of SEQ ID NO:124, 243 and 249. In one embodiment, the antibody comprises: (a) a heavy chain variable (V_{H}) region comprising: (1) a V_{H} CDR1 having the amino acid sequence of SEQ ID NO:111; (2) a V_{H} CDR2 having the amino acid sequence of SEQ ID NO:114; and (3) a V_{H} CDR3 having the amino acid sequence of SEQ ID NO:118; and (b) a light chain variable (V_{L}) region comprising: (1) a V_{L} CDR1 having the amino acid sequence of SEQ ID NO:94; (2) a V_{L} CDR2 having the amino acid sequence of SEQ ID NO:83; and (3) a V_{L} CDR3 having the amino acid sequence of SEQ ID NO:124. In one embodiment, the antibody comprises: (a) a heavy chain variable (V_{H}) region comprising: (1) a V_{H} CDR1 having the amino acid sequence of SEQ ID NO:234; (2) a V_{H} CDR2 having the amino acid sequence of SEQ ID NO:223; and (3) a V_{H} CDR3 having the amino acid sequence of SEQ ID NO:236; and (b) a light chain variable (V_{L}) region comprising: (1) a V_{L} CDR1 having the amino acid sequence of SEQ ID NO:237; (2) a V_{L} CDR2 having the amino acid sequence of SEQ ID NO:238; and (3) a V_{L} CDR3 having the amino acid sequence of SEQ ID NO:124. In one embodiment, the antibody comprises: (a) a heavy chain variable (V_{H}) region comprising: (1) a V_{H} CDR1 having the amino acid sequence of SEQ ID NO:240; (2) a V_{H} CDR2 having the amino acid sequence of SEQ ID NO:239; and (3) a V_{H} CDR3 having the amino acid sequence of SEQ ID NO:241; and (b) a light chain variable (V_{L}) region comprising: (1) a V_{L} CDR1 having the amino acid sequence of SEQ ID NO:242; (2) a V_{L} CDR2 having the amino acid sequence of SEQ ID NO:83; and (3) a V_{L} CDR3 having the amino acid sequence of SEQ ID NO:243. In one embodiment, the antibody comprises: (a) a heavy chain variable (V_{H}) region comprising: (1) a V_{H} CDR1 having the amino acid sequence of SEQ ID NO:244; (2) a V_{H} CDR2 having the amino acid sequence of SEQ ID NO:252; and (3) a V_{H} CDR3 having the amino acid sequence of SEQ ID NO:246; and (b) a light chain variable (V_{L}) region comprising: (1) a V_{L} CDR1 having the amino acid sequence of SEQ ID NO:247; (2) a V_{L} CDR2 having the amino acid sequence of SEQ ID NO:248; and (3) a V_{L} CDR3 having the amino acid sequence of SEQ ID NO:249. In one embodiment, the antibody comprises: (a) a heavy chain variable (V_{H}) region comprising: (1) a V_{H} CDR1 having the amino acid sequence of SEQ ID NO:250; (2) a V_{H} CDR2 having the amino acid sequence of SEQ ID NO:253; and (3) a V_{H} CDR3 having the amino acid sequence of SEQ ID NO:236; and (b) a light chain variable (V_{L}) region comprising: (1) a V_{L} CDR1 having the amino acid sequence of SEQ ID NO:237; (2) a V_{L} CDR2 having the amino acid sequence of SEQ ID NO:238; and (3) a V_{L} CDR3 having the amino acid sequence of SEQ ID NO:124. In one embodiment, the antibody comprises: (a) a heavy chain variable (V_{H}) region comprising: (1) a V_{H} CDR1 having an amino acid sequence of selected from the group consisting of SEQ ID NO:111, 234, 240, 244, and 250; (2) a V_{H} CDR2 having an amino acid sequence of selected from the group consisting of SEQ ID NO:115, 254, 239, 259, and 262; and (3) a V_{H} CDR3 having an amino acid sequence of selected from the group consisting of SEQ ID NO:119, 255, 257, and 260; and (b) a light chain variable (V_{L}) region comprising: (1) a V_{L} CDR1 having an amino acid sequence of selected from the group consisting of SEQ ID NO:122, 256, 258, and 261; (2) a V_{L} CDR2 having an amino acid sequence of selected from the group consisting of SEQ ID NO:83, 238, and 248; and (3) a V_{L} CDR3 having an amino acid sequence of selected from the group consisting of SEQ ID NO:124, 243, and 249.

In some embodiments, the invention provides an isolated antibody or functional fragment thereof that binds to C16orf54, wherein the antibody comprises a heavy chain variable (V_{H}) region comprising one, two, three or more VH CDR amino acid sequences of Table 21, and/or a light chain variable (V_{L}) region comprising one, two, three or more VL CDR amino acid sequences of Table 21. Accordingly, in some embodiments, the antibody comprises: (a) a heavy chain variable (V_{H}) region comprising: (1) a V_{H} CDR1 having the amino acid sequence of SEQ ID NO:111; (2) a V_{H} CDR2 having the amino acid sequence of SEQ ID NO:115; and (3) a V_{H} CDR3 having the amino acid sequence of SEQ ID NO:119; and (b) a light chain variable (V_{L}) region comprising: (1) a V_{L} CDR1 having the amino acid sequence of SEQ ID NO:122; (2) a V_{L} CDR2 having the amino acid sequence of SEQ ID NO:83; and (3) a V_{L} CDR3 having the amino acid sequence of SEQ ID NO:124. In one embodiment, the antibody comprises: (a) a heavy chain variable (V_{H}) region comprising: (1) a V_{H} CDR1 having the amino acid sequence of SEQ ID NO:234; (2) a V_{H} CDR2 having the amino acid sequence of SEQ ID NO:254; and (3) a V_{H} CDR3 having the amino acid sequence of SEQ ID NO:255; and (b) a light chain variable (V_{L}) region comprising: (1) a V_{L} CDR1 having the amino acid sequence of SEQ ID NO:256; (2) a V_{L} CDR2 having the amino acid sequence of SEQ ID NO:238; and (3) a V_{L} CDR3 having the amino acid sequence of SEQ ID NO:124. In one embodiment, the antibody comprises: (a) a heavy chain variable (V_{H}) region comprising: (1) a V_{H} CDR1 having the amino acid sequence of SEQ ID NO:240; (2) a V_{H} CDR2 having the amino acid sequence of SEQ ID NO:239; and (3) a V_{H} CDR3 having the amino acid sequence of SEQ ID NO:257; and (b) a light chain variable (V_{L}) region comprising: (1) a V_{L} CDR1 having the amino acid sequence of SEQ ID NO:258; (2) a V_{L} CDR2 having the amino acid sequence of SEQ ID NO:83; and (3) a V_{L} CDR3 having the amino acid sequence of SEQ ID NO:243. In one embodiment, the antibody comprises: (a) a heavy chain variable (V_{H}) region comprising: (1) a V_{H} CDR1 having the amino acid sequence of SEQ ID NO:244; (2) a V_{H} CDR2 having the amino acid sequence of SEQ ID NO:259; and (3) a V_{H} CDR3 having the amino acid sequence of SEQ ID NO:260; and (b) a light chain variable (V_{L}) region comprising: (1) a V_{L} CDR1 having the amino acid sequence of SEQ ID NO:261; (2) a V_{L} CDR2 having the amino acid sequence of SEQ ID NO:248; and (3) a V_{L} CDR3 having the amino acid sequence of SEQ ID NO:249. In one embodiment, the antibody comprises: (a) a heavy chain variable (V_{H}) region comprising: (1) a V_{H} CDR1 having the amino acid sequence of SEQ ID NO:250; (2) a V_{H} CDR2 having the amino acid sequence of SEQ ID NO:262; and (3) a V_{H} CDR3 having the amino acid sequence of SEQ ID NO:255; and (b) a light chain variable (V_{L}) region comprising: (1) a V_{L} CDR1 having the amino acid sequence of SEQ ID NO:256; (2) a V_{L} CDR2 having the amino acid sequence of SEQ ID NO:238; and (3) a V_{L} CDR3 having the amino acid sequence of SEQ ID NO:124.

In some embodiments, the invention provides an isolated antibody or functional fragment thereof that binds to C16orf54, wherein the antibody comprises a heavy chain variable (V_{H}) region comprising one, two, three or more VH CDR amino acid sequences of Table 22, and/or a light chain variable (V_{L}) region comprising one, two, three or more VL CDR amino acid sequences of Table 22. Accordingly, in some embodiments, the antibody comprises: (a) a heavy chain variable (V_{H}) region comprising: (1) a V_{H} CDR1 having an amino acid sequence of selected from the group consisting of SEQ ID NO:111, 234, 240, 244, and 250; (2) a V_{H} CDR2 having an amino acid sequence of selected from the group consisting of SEQ ID NO:116, 263, 239, 270, and 273; and (3) a V_{H} CDR3 having an amino acid sequence of selected from the group consisting of SEQ ID NO:120, 264, 267, and 271; and (b) a light chain variable (V_{L}) region comprising: (1) a V_{L} CDR1 having an amino acid sequence of selected from the group consisting of SEQ ID NO:123, 265, 268, and 247; (2) a V_{L} CDR2 having an amino acid sequence of selected from the group consisting of SEQ ID NO:83, 238, and 248; and (3) a V_{L} CDR3 having an amino acid sequence of selected from the group consisting of SEQ ID NO:125, 269, and 272. In one embodiment, the antibody comprises: (a) a heavy chain variable (V_{H}) region comprising: (1) a V_{H} CDR1 having the amino acid sequence of SEQ ID NO:111; (2) a V_{H} CDR2 having the amino acid sequence of SEQ ID NO:116; and (3) a V_{H} CDR3 having the amino acid sequence of SEQ ID NO:120; and (b) a light chain variable (V_{L}) region comprising: (1) a V_{L} CDR1 having the amino acid sequence of SEQ ID NO:123; (2) a V_{L} CDR2 having the amino acid sequence of SEQ ID NO:83; and (3) a V_{L} CDR3 having the amino acid sequence of SEQ ID NO:125. In one embodiment, the antibody comprises: (a) a heavy chain variable (V_{H}) region comprising: (1) a V_{H} CDR1 having the amino acid sequence of SEQ ID NO:234; (2) a V_{H} CDR2 having the amino acid sequence of SEQ ID NO:263; and (3) a V_{H} CDR3 having the amino acid sequence of SEQ ID NO:264; and (b) a light chain variable (V_{L}) region comprising: (1) a V_{L} CDR1 having the amino acid sequence of SEQ ID NO:265; (2) a V_{L} CDR2 having the amino acid sequence of SEQ ID NO:238; and (3) a V_{L} CDR3 having the amino acid sequence of SEQ ID NO:125. In one embodiment, the antibody comprises: (a) a heavy chain variable (V_{H}) region comprising: (1) a V_{H} CDR1 having the amino acid sequence of SEQ ID NO:240; (2) a V_{H} CDR2 having the amino acid sequence of SEQ ID NO:239; and (3) a V_{H} CDR3 having the amino acid sequence of SEQ ID NO:267; and (b) a light chain variable (V_{L}) region comprising: (1) a V_{L} CDR1 having the amino acid sequence of SEQ ID NO:268; (2) a V_{L} CDR2 having the amino acid sequence of SEQ ID NO:83; and (3) a V_{L} CDR3 having the amino acid sequence of SEQ ID NO:269. In one embodiment, the antibody comprises: (a) a heavy chain variable (V_{H}) region comprising: (1) a V_{H} CDR1 having the amino acid sequence of SEQ ID NO:244; (2) a V_{H} CDR2 having the amino acid sequence of SEQ ID NO:270; and (3) a V_{H} CDR3 having the amino acid sequence of SEQ ID NO:271; and (b) a light chain variable (V_{L}) region comprising: (1) a V_{L} CDR1 having the amino acid sequence of SEQ ID NO:247; (2) a V_{L} CDR2 having the amino acid sequence of SEQ ID NO:248; and (3) a V_{L} CDR3 having the amino acid sequence of SEQ ID NO:272. In one embodiment, the antibody comprises: (a) a heavy chain variable (V_{H}) region comprising: (1) a V_{H} CDR1 having the amino acid sequence of SEQ ID NO:250; (2) a V_{H} CDR2 having the amino acid sequence of SEQ ID NO:273; and (3) a V_{H} CDR3 having the amino acid sequence of SEQ ID NO:264; and (b) a light chain variable (V_{L}) region comprising: (1) a V_{L} CDR1 having the amino acid sequence of SEQ ID NO:265; (2) a V_{L} CDR2 having the amino acid sequence of SEQ ID NO:238; and (3) a V_{L} CDR3 having the amino acid sequence of SEQ ID NO:125.

In some embodiments, the invention provides an isolated antibody or functional fragment thereof that binds to C16orf54, wherein the antibody comprises a heavy chain variable (V_{H}) region comprising one, two, three or more VH CDR amino acid sequences of Table 23, and/or a light chain variable (V_{L}) region comprising one, two, three or more VL CDR amino acid sequences of Table 23. Accordingly, in some embodiments, the antibody comprises: (a) a heavy chain variable (V_{H}) region comprising: (1) a V_{H} CDR1 having an amino acid sequence of selected from the group consisting of SEQ ID NO:112, 274, 266, 277, and 279; (2) a V_{H} CDR2 having an amino acid sequence of selected from the group consisting of SEQ ID NO:117, 275, 239, 278, and 280; and (3) a V_{H} CDR3 having an amino acid sequence of SEQ ID NO:121 or 276. In one embodiment, the antibody comprises: (a) a heavy chain variable (V_{H}) region comprising: (1) a V_{H} CDR1 having the amino acid sequence of SEQ ID NO:112; (2) a V_{H} CDR2 having the amino acid sequence of SEQ ID NO:117; and (3) a V_{H} CDR3 having the amino acid sequence of SEQ ID NO:121. In one embodiment, the antibody comprises: (a) a heavy chain variable (V_{H}) region comprising: (1) a V_{H} CDR1 having the amino acid sequence of SEQ ID NO:274; (2) a V_{H} CDR2 having the amino acid sequence of SEQ ID NO:275; and (3) a V_{H} CDR3 having the amino acid sequence of SEQ ID NO:276. In one embodiment, the antibody comprises: (a) a heavy chain variable (V_{H}) region comprising: (1) a V_{H} CDR1 having the amino acid sequence of SEQ ID NO:266; (2) a V_{H} CDR2 having the amino acid sequence of SEQ ID NO:239; and (3) a V_{H} CDR3 having the amino acid sequence of SEQ ID NO:276. In one embodiment, the antibody comprises: (a) a heavy chain variable (V_{H}) region comprising: (1) a V_{H} CDR1 having the amino acid sequence of SEQ ID NO:277; (2) a V_{H} CDR2 having the amino acid sequence of SEQ ID NO:278; and (3) a V_{H} CDR3 having the amino acid sequence of SEQ ID NO:121. In one embodiment, the antibody comprises: (a) a heavy chain variable (V_{H}) region comprising: (1) a V_{H} CDR1 having the amino acid sequence of SEQ ID NO:279; (2) a V_{H} CDR2 having the amino acid sequence of SEQ ID NO:280; and (3) a V_{H} CDR3 having the amino acid sequence of SEQ ID NO:276.

In some embodiments, the invention provides an isolated antibody or functional fragment thereof that binds to C16orf54, wherein the antibody comprises a heavy chain variable (V_{H}) region comprising one, two, three or more VH CDR amino acid sequences of Table 24, and/or a light chain variable (V_{L}) region comprising one, two, three or more VL CDR amino acid sequences of Table 24. Accordingly, in some embodiments, the antibody comprises: (a) a heavy chain variable (V_{H}) region comprising: (1) a V_{H} CDR1 having an amino acid sequence of selected from the group consisting of SEQ ID NO:126, 281, 285, 289, and 294; (2) a V_{H} CDR2 having an amino acid sequence of selected from the group consisting of SEQ ID NO:128, 282, 162, 290, and 295; and (3) a V_{H} CDR3 having an amino acid sequence of selected from the group consisting of SEQ ID NO:130, 283, 286, and 291; and (b) a light chain variable (V_{L}) region comprising: (1) a V_{L} CDR1 having an amino acid sequence of selected from the group consisting of SEQ ID NO:88, 284, 287, 292, and 284; (2) a V_{L} CDR2 having an amino acid sequence of selected from the group consisting of SEQ ID NO:71, 160, and 170; and (3) a V_{L} CDR3 having an amino acid sequence of selected from the group consisting of SEQ ID NO:90, 288 and 293. In one embodiment, the antibody comprises: (a) a heavy chain variable (V_{H}) region comprising: (1) a V_{H} CDR1 having the amino acid sequence of SEQ ID NO:126; (2) a V_{H} CDR2 having the amino acid sequence of SEQ ID NO:128; and (3) a V_{H} CDR3 having the amino acid sequence of SEQ ID NO:130; and (b) a light chain variable (V_{L}) region comprising: (1) a V_{L} CDR1 having the amino acid sequence of SEQ ID NO:88; (2) a V_{L} CDR2 having the amino acid sequence of SEQ ID NO:71; and (3) a V_{L} CDR3 having the amino acid sequence of SEQ ID NO:90. In one embodiment, the antibody comprises: (a) a heavy chain variable (V_{H}) region comprising: (1) a V_{H} CDR1 having the amino acid sequence of SEQ ID NO:281; (2) a V_{H} CDR2 having the amino acid sequence of SEQ ID NO:282; and (3) a V_{H} CDR3 having the amino acid sequence of SEQ ID NO:283; and (b) a light chain variable (V_{L}) region comprising: (1) a V_{L} CDR1 having the amino acid sequence of SEQ ID NO:284; (2) a V_{L} CDR2 having the amino acid sequence of SEQ ID NO:160; and (3) a V_{L} CDR3 having the amino acid sequence of SEQ ID NO:90. In one embodiment, the antibody comprises: (a) a heavy chain variable (V_{H}) region comprising: (1) a V_{H} CDR1 having the amino acid sequence of SEQ ID NO:285; (2) a V_{H} CDR2 having the amino acid sequence of SEQ ID NO:162; and (3) a V_{H} CDR3 having the amino acid sequence of SEQ ID NO:286; and (b) a light chain variable (V_{L}) region comprising: (1) a V_{L} CDR1 having the amino acid sequence of SEQ ID NO:287; (2) a V_{L} CDR2 having the amino acid sequence of SEQ ID NO:71; and (3) a V_{L} CDR3 having the amino acid sequence of SEQ ID NO:288. In one embodiment, the antibody comprises: (a) a heavy chain variable (V_{H}) region comprising: (1) a V_{H} CDR1 having the amino acid sequence of SEQ ID NO:289; (2) a V_{H} CDR2 having the amino acid sequence of SEQ ID NO:290; and (3) a V_{H} CDR3 having the amino acid sequence of SEQ ID NO:291; and (b) a light chain variable (V_{L}) region comprising: (1) a V_{L} CDR1 having the amino acid sequence of SEQ ID NO:292; (2) a V_{L} CDR2 having the amino acid sequence of SEQ ID NO:170; and (3) a V_{L} CDR3 having the amino acid sequence of SEQ ID NO:293. In one embodiment, the antibody comprises: (a) a heavy chain variable (V_{H}) region comprising: (1) a V_{H}CDR1 having the amino acid sequence of SEQ ID NO:294; (2) a V_{H} CDR2 having the amino acid sequence of SEQ ID NO:295; and (3) a V_{H} CDR3 having the amino acid sequence of SEQ ID NO:283; and (b) a light chain variable (V_{L}) region comprising: (1) a V_{L} CDR1 having the amino acid sequence of SEQ ID NO:284; (2) a V_{L} CDR2 having the amino acid sequence of SEQ ID NO:160; and (3) a V_{L} CDR3 having the amino acid sequence of SEQ ID NO:90.

In some embodiments, the invention provides an isolated antibody or functional fragment thereof that binds to C16orf54, wherein the antibody comprises a heavy chain variable (V_{H}) region comprising one, two, three or more VH CDR amino acid sequences of Table 25, and/or a light chain variable (V_{L}) region comprising one, two, three or more VL CDR amino acid sequences of Table 25. Accordingly, in some embodiments, the antibody comprises: (a) a heavy chain variable (V_{H}) region comprising: (1) a V_{H} CDR1 having an amino acid sequence of selected from the group consisting of SEQ ID NO:126, 281, 285, 289, and 294; (2) a V_{H} CDR2 having an amino acid sequence of selected from the group consisting of SEQ ID NO:128, 282, 162, 290, and 295; and (3) a V_{H} CDR3 having an amino acid sequence of selected from the group consisting of SEQ ID NO:130, 283, 286, and 291; and (b) a light chain variable (V_{L}) region comprising: (1) a V_{L} CDR1 having an amino acid sequence of selected from the group consisting of SEQ ID NO:88, 284, 287, and 292; (2) a V_{L} CDR2 having an amino acid sequence of selected from the group consisting of SEQ ID NO:71, 160, and 170; and (3) a V_{L} CDR3 having an amino acid sequence of selected from the group consisting of SEQ ID NO:90, 288 and 293. In one embodiment, the antibody comprises: (a) a heavy chain variable (V_{H}) region comprising: (1) a V_{H} CDR1 having the amino acid sequence of SEQ ID NO:126; (2) a V_{H} CDR2 having the amino acid sequence of SEQ ID NO:128; and (3) a V_{H} CDR3 having the amino acid sequence of SEQ ID NO:130; and (b) a light chain variable (V_{L}) region comprising: (1) a V_{L} CDR1 having the amino acid sequence of SEQ ID NO:88; (2) a V_{L} CDR2 having the amino acid sequence of SEQ ID NO:71; and (3) a V_{L} CDR3 having the amino acid sequence of SEQ ID NO:90. In one embodiment, the antibody comprises: (a) a heavy chain variable (V_{H}) region comprising: (1) a V_{H} CDR1 having the amino acid sequence of SEQ ID NO:281; (2) a V_{H} CDR2 having the amino acid sequence of SEQ ID NO:282; and (3) a V_{H} CDR3 having the amino acid sequence of SEQ ID NO:283; and (b) a light chain variable (V_{L}) region comprising: (1) a V_{L} CDR1 having the amino acid sequence of SEQ ID NO:284; (2) a V_{L} CDR2 having the amino acid sequence of SEQ ID NO:160; and (3) a V_{L} CDR3 having the amino acid sequence of SEQ ID NO:90. In one embodiment, the antibody comprises: (a) a heavy chain variable (V_{H}) region comprising: (1) a V_{H} CDR1 having the amino acid sequence of SEQ ID NO:285; (2) a V_{H} CDR2 having the amino acid sequence of SEQ ID NO:162; and (3) a V_{H} CDR3 having the amino acid sequence of SEQ ID NO:286; and (b) a light chain variable (V_{L}) region comprising: (1) a V_{L} CDR1 having the amino acid sequence of SEQ ID NO:287; (2) a V_{L} CDR2 having the amino acid sequence of SEQ ID NO:71; and (3) a V_{L} CDR3 having the amino acid sequence of SEQ ID NO:288. In one embodiment, the antibody comprises: (a) a heavy chain variable (V_{H}) region comprising: (1) a V_{H} CDR1 having the amino acid sequence of SEQ ID NO:289; (2) a V_{H} CDR2 having the amino acid sequence of SEQ ID NO:290; and (3) a V_{H} CDR3 having the amino acid sequence of SEQ ID NO:291; and (b) a light chain variable (V_{L}) region comprising: (1) a V_{L} CDR1 having the amino acid sequence of SEQ ID NO:292; (2) a V_{L} CDR2 having the amino acid sequence of SEQ ID NO:170; and (3) a V_{L} CDR3 having the amino acid sequence of SEQ ID NO:293. In one embodiment, the antibody comprises: (a) a heavy chain variable (V_{H}) region comprising: (1) a V_{H} CDR1 having the amino acid sequence of SEQ ID NO:294; (2) a V_{H} CDR2 having the amino acid sequence of SEQ ID NO:295; and (3) a V_{H} CDR3 having the amino acid sequence of SEQ ID NO:283; and (b) a light chain variable (V_{L}) region comprising: (1) a V_{L} CDR1 having the amino acid sequence of SEQ ID NO:284; (2) a V_{L} CDR2 having the amino acid sequence of SEQ ID NO:160; and (3) a V_{L} CDR3 having the amino acid sequence of SEQ ID NO:90.

In some embodiments, the invention provides an isolated antibody or functional fragment thereof that binds to C16orf54, wherein the antibody comprises a heavy chain variable (V_{H}) region comprising one, two, three or more VH CDR amino acid sequences of Table 26, and/or a light chain variable (V_{L}) region comprising one, two, three or more VL CDR amino acid sequences of Table 26. Accordingly, in some embodiments, the antibody comprises: (a) a heavy chain variable (V_{H}) region comprising: (1) a V_{H} CDR1 having an amino acid sequence of selected from the group consisting of SEQ ID NO:126, 281, 285, 289, and 294; (2) a V_{H} CDR2 having an amino acid sequence of selected from the group consisting of SEQ ID NO:128, 282, 162, 290, and 295; and (3) a V_{H} CDR3 having an amino acid sequence of selected from the group consisting of SEQ ID NO:77, 296, 286, and 291. In one embodiment, the antibody comprises: (a) a heavy chain variable (V_{H}) region comprising: (1) a V_{H} CDR1 having the amino acid sequence of SEQ ID NO:126; (2) a V_{H} CDR2 having the amino acid sequence of SEQ ID NO:128; and (3) a V_{H} CDR3 having the amino acid sequence of SEQ ID NO:77. In one embodiment, the antibody comprises: (a) a heavy chain variable (V_{H}) region comprising: (1) a V_{H} CDR1 having the amino acid sequence of SEQ ID NO:281; (2) a V_{H} CDR2 having the amino acid sequence of SEQ ID NO:282; and (3) a V_{H} CDR3 having the amino acid sequence of SEQ ID NO:296. In one embodiment, the antibody comprises: (a) a heavy chain variable (V_{H}) region comprising: (1) a V_{H} CDR1 having the amino acid sequence of SEQ ID NO:285; (2) a V_{H} CDR2 having the amino acid sequence of SEQ ID NO:162; and (3) a V_{H} CDR3 having the amino acid sequence of SEQ ID NO:286. In one embodiment, the antibody comprises: (a) a heavy chain variable (V_{H}) region comprising: (1) a V_{H} CDR1 having the amino acid sequence of SEQ ID NO:289; (2) a V_{H} CDR2 having the amino acid sequence of SEQ ID NO:290; and (3) a V_{H} CDR3 having the amino acid sequence of SEQ ID NO:291. In one embodiment, the antibody comprises: (a) a heavy chain variable (V_{H}) region comprising: (1) a V_{H} CDR1 having the amino acid sequence of SEQ ID NO:294; (2) a V_{H} CDR2 having the amino acid sequence of SEQ ID NO:295; and (3) a V_{H} CDR3 having the amino acid sequence of SEQ ID NO:296.

In some embodiments, the invention provides an isolated antibody or functional fragment thereof that binds to C16orf54, wherein the antibody comprises a heavy chain variable (V_{H}) region comprising one, two, three or more VH CDR amino acid sequences of Table 27, and/or a light chain variable (V_{L}) region comprising one, two, three or more VL CDR amino acid sequences of Table 27. Accordingly, in some embodiments, the antibody comprises: (a) a heavy chain variable (V_{H}) region comprising: (1) a V_{H} CDR1 having an amino acid sequence of selected from the group consisting of SEQ ID NO:127, 297, 285, 299, and 301; (2) a V_{H} CDR2 having an amino acid sequence of selected from the group consisting of SEQ ID NO:129, 298, 162, 300, and 302; and (3) a V_{H} CDR3 having an amino acid sequence of selected from the group consisting of SEQ ID NO:130, 283, 286, and 291. In one embodiment, the antibody comprises: (a) a heavy chain variable (V_{H}) region comprising: (1) a V_{H} CDR1 having the amino acid sequence of SEQ ID NO:127; (2) a V_{H} CDR2 having the amino acid sequence of SEQ ID NO:129; and (3) a V_{H} CDR3 having the amino acid sequence of SEQ ID NO:130. In one embodiment, the antibody comprises: (a) a heavy chain variable (V_{H}) region comprising: (1) a V_{H} CDR1 having the amino acid sequence of SEQ ID NO:297; (2) a V_{H} CDR2 having the amino acid sequence of SEQ ID NO:298; and (3) a V_{H} CDR3 having the amino acid sequence of SEQ ID NO:283. In one embodiment, the antibody comprises: (a) a heavy chain variable (V_{H}) region comprising: (1) a V_{H} CDR1 having the amino acid sequence of SEQ ID NO:285; (2) a V_{H} CDR2 having the amino acid sequence of SEQ ID NO:162; and (3) a V_{H} CDR3 having the amino acid sequence of SEQ ID NO:286. In one embodiment, the antibody comprises: (a) a heavy chain variable (V_{H}) region comprising: (1) a V_{H} CDR1 having the amino acid sequence of SEQ ID NO:299; (2) a V_{H} CDR2 having the amino acid sequence of SEQ ID NO:300; and (3) a V_{H} CDR3 having the amino acid sequence of SEQ ID NO:291. In one embodiment, the antibody comprises: (a) a heavy chain variable (V_{H}) region comprising: (1) a V_{H} CDR1 having the amino acid sequence of SEQ ID NO:301; (2) a V_{H} CDR2 having the amino acid sequence of SEQ ID NO:302; and (3) a V_{H} CDR3 having the amino acid sequence of SEQ ID NO:283.

In some embodiments, the invention provides an isolated antibody or functional fragment thereof that binds to C16orf54, wherein the antibody comprises a heavy chain variable (V_{H}) region comprising one, two, three or more VH CDR amino acid sequences of Table 28, and/or a light chain variable (V_{L}) region comprising one, two, three or more VL CDR amino acid sequences of Table 28. Accordingly, in some embodiments, the antibody comprises: (a) a heavy chain variable (V_{H}) region comprising: (1) a V_{H} CDR1 having an amino acid sequence of selected from the group consisting of SEQ ID NO:126, 281, 285, 289, and 294; (2) a V_{H} CDR2 having an amino acid sequence of selected from the group consisting of SEQ ID NO:128, 282, 162, 290, and 295; and (3) a V_{H} CDR3 having an amino acid sequence of selected from the group consisting of SEQ ID NO:130, 283, 286, and 291. In one embodiment, the antibody comprises: (a) a heavy chain variable (V_{H}) region comprising: (1) a V_{H} CDR1 having the amino acid sequence of SEQ ID NO:126; (2) a V_{H} CDR2 having the amino acid sequence of SEQ ID NO:128; and (3) a V_{H} CDR3 having the amino acid sequence of SEQ ID NO:130. In one embodiment, the antibody comprises: (a) a heavy chain variable (V_{H}) region comprising: (1) a V_{H} CDR1 having the amino acid sequence of SEQ ID NO:281; (2) a V_{H} CDR2 having the amino acid sequence of SEQ ID NO:282; and (3) a V_{H} CDR3 having the amino acid sequence of SEQ ID NO:283. In one embodiment, the antibody comprises: (a) a heavy chain variable (V_{H}) region comprising: (1) a V_{H} CDR1 having the amino acid sequence of SEQ ID NO:285; (2) a V_{H} CDR2 having the amino acid sequence of SEQ ID NO:162; and (3) a V_{H} CDR3 having the amino acid sequence of SEQ ID NO:286. In one embodiment, the antibody comprises: (a) a heavy chain variable (V_{H}) region comprising: (1) a V_{H} CDR1 having the amino acid sequence of SEQ ID NO:289; (2) a V_{H} CDR2 having the amino acid sequence of SEQ ID NO:290; and (3) a V_{H} CDR3 having the amino acid sequence of SEQ ID NO:291. In one embodiment, the antibody comprises: (a) a heavy chain variable (V_{H}) region comprising: (1) a V_{H} CDR1 having the amino acid sequence of SEQ ID NO:294; (2) a V_{H} CDR2 having the amino acid sequence of SEQ ID NO:295; and (3) a V_{H} CDR3 having the amino acid sequence of SEQ ID NO:283.

In some embodiments, the invention provides an isolated antibody or functional fragment thereof that binds to C16orf54, wherein the antibody comprises a heavy chain variable (V_{H}) region comprising one, two, three or more VH CDR amino acid sequences of Table 29, and/or a light chain variable (V_{L}) region comprising one, two, three or more VL CDR amino acid sequences of Table 29. Accordingly, in some embodiments, the antibody comprises: (a) a heavy chain variable (V_{H}) region comprising: (1) a V_{H} CDR1 having an amino acid sequence of selected from the group consisting of SEQ ID NO:91, 303, 307, 311, and 317; (2) a V_{H} CDR2 having an amino acid sequence of selected from the group consisting of SEQ ID NO:92, 304, 308, 312, and 316; and (3) a V_{H} CDR3 having an amino acid sequence of selected from the group consisting of SEQ ID NO:93, 305, 309, and 313; and (b) a light chain variable (V_{L}) region comprising: (1) a V_{L} CDR1 having an amino acid sequence of selected from the group consisting of SEQ ID NO:94, 237, 242, and 247; (2) a V_{L} CDR2 having an amino acid sequence of selected from the group consisting of SEQ ID NO:83, 306, and 314; and (3) a V_{L} CDR3 having an amino acid sequence of selected from the group consisting of SEQ ID NO:96, 310, and 315. In one embodiment, the antibody comprises: (a) a heavy chain variable (V_{H}) region comprising: (1) a V_{H} CDR1 having the amino acid sequence of SEQ ID NO:91; (2) a V_{H} CDR2 having the amino acid sequence of SEQ ID NO:92; and (3) a V_{H} CDR3 having the amino acid sequence of SEQ ID NO:93; and (b) a light chain variable (V_{L}) region comprising: (1) a V_{L} CDR1 having the amino acid sequence of SEQ ID NO:94; (2) a V_{L} CDR2 having the amino acid sequence of SEQ ID NO:83; and (3) a V_{L} CDR3 having the amino acid sequence of SEQ ID NO:96. In one embodiment, the antibody comprises: (a) a heavy chain variable (V_{H}) region comprising: (1) a V_{H} CDR1 having the amino acid sequence of SEQ ID NO:303; (2) a V_{H} CDR2 having the amino acid sequence of SEQ ID NO:304; and (3) a V_{H} CDR3 having the amino acid sequence of SEQ ID NO:305; and (b) a light chain variable (V_{L}) region comprising: (1) a V_{L} CDR1 having the amino acid sequence of SEQ ID NO:237; (2) a V_{L} CDR2 having the amino acid sequence of SEQ ID NO:306; and (3) a V_{L} CDR3 having the amino acid sequence of SEQ ID NO:96. In one embodiment, the antibody comprises: (a) a heavy chain variable (V_{H}) region comprising: (1) a V_{H} CDR1 having the amino acid sequence of SEQ ID NO:307; (2) a V_{H} CDR2 having the amino acid sequence of SEQ ID NO:308; and (3) a V_{H} CDR3 having the amino acid sequence of SEQ ID NO:309; and (b) a light chain variable (V_{L}) region comprising: (1) a V_{L} CDR1 having the amino acid sequence of SEQ ID NO:242; (2) a V_{L} CDR2 having the amino acid sequence of SEQ ID NO:83; and (3) a V_{L} CDR3 having the amino acid sequence of SEQ ID NO:310. In one embodiment, the antibody comprises: (a) a heavy chain variable (V_{H}) region comprising: (1) a V_{H} CDR1 having the amino acid sequence of SEQ ID NO:311; (2) a V_{H} CDR2 having the amino acid sequence of SEQ ID NO:312; and (3) a V_{H} CDR3 having the amino acid sequence of SEQ ID NO:313; and (b) a light chain variable (V_{L}) region comprising: (1) a V_{L} CDR1 having the amino acid sequence of SEQ ID NO:247; (2) a V_{L} CDR2 having the amino acid sequence of SEQ ID NO:314; and (3) a V_{L} CDR3 having the amino acid sequence of SEQ ID NO:315. In one embodiment, the antibody comprises: (a) a heavy chain variable (V_{H}) region comprising: (1) a V_{H} CDR1 having the amino acid sequence of SEQ ID NO:317; (2) a V_{H} CDR2 having the amino acid sequence of SEQ ID NO:316; and (3) a V_{H} CDR3 having the amino acid sequence of SEQ ID NO:305; and (b) a light chain variable (V_{L}) region comprising: (1) a V_{L} CDR1 having the amino acid sequence of SEQ ID NO:237; (2) a V_{L} CDR2 having the amino acid sequence of SEQ ID NO:306; and (3) a V_{L} CDR3 having the amino acid sequence of SEQ ID NO:96.

In some embodiments, the invention provides an isolated antibody or a functional fragment thereof that specifically binds to amino acid residues 1-31 of SEQ ID NO:1. In some embodiments, the invention provides an isolated antibody or a functional fragment thereof that specifically binds to amino acid residues 1-15 of SEQ ID NO:1 or amino acid residues 9-24 of SEQ ID NO:1.

In some embodiments, an antibody provided herein is a monoclonal antibody. In some embodiments, the monoclonal antibody of the invention is a humanized, human or chimeric antibody. In some embodiments, the antibody functional fragment of the invention is an Fab, Fab′, F(ab′)2, Fv, scFv, (scFv)2, single chain antibody molecule, dual variable domain antibody, single variable domain antibody, linear antibody, V domain, or a multspecific antibody formed from antibody fragments.

In a further embodiment, the invention comprises a binding agent that binds to essentially the same epitope as any of the antibodies disclosed above. In some embodiments, the binding agent inhibits the growth of a tumor expressing C16orf54. In some embodiments, the binding agent is an antibody or a functional fragment thereof. In other embodiments, the binding agent is an anticalin, an adnectin, an affibody, a DARPin, a fynomer, an affitin, an affilin, an avimer, a cysteine-rich knottin peptide, or an engineered Kunitz-type inhibitor.

In one embodiment, the invention provides a binding agent capable of binding to C16orf54, wherein any one of the antibodies disclosed above displaces the binding agent in a competitive binding assay. In some embodiments, the binding agent is an antibody, or a functional fragment thereof. In another embodiment, the invention provides a binding agent capable of binding to C16orf54, wherein the binding agent displaces any one of the antibodies disclosed above in a competitive binding assay. In some embodiments, the binding agent is an antibody, or a functional fragment thereof.

In an embodiment, the invention provides humanized antibodies.

In some embodiments, the invention provides an antibody that binds to C16orf54, wherein the antibody comprises a heavy chain variable domain having at least 90%, at least 91%, at least 92%, at least 93%, at least 94%, at least 95%, at least 96%, at least 97%, at least 98%, or at least 99% sequence identity to an amino acid sequence selected from the group consisting of SEQ ID NO: 4, SEQ ID NO: 8, SEQ ID NO:12, SEQ ID NO:16, SEQ ID NO:20, SEQ ID NO:24, SEQ ID NO:28, SEQ ID NO:32, SEQ ID NO:36, SEQ ID NO:40, SEQ ID NO:44, SEQ ID NO:48, SEQ ID NO:52, SEQ ID NO:56, SEQ ID NO:60, SEQ ID NO:64, SEQ ID NO:132, SEQ ID NO:134, SEQ ID NO:136, SEQ ID NO:138, SEQ ID NO:140, SEQ ID NO:142, SEQ ID NO:144, and SEQ ID NO:146. In some embodiments, the antibody comprises a light chain variable domain having at least 90%, at least 91%, at least 92%, at least 93%, at least 94%, at least 95%, at least 96%, at least 97%, at least 98%, or at least 99% sequence identity to an amino acid sequence selected from the group consisting of SEQ ID NO: 6, SEQ ID NO: 10, SEQ ID NO:14, SEQ ID NO:18, SEQ ID NO:22, SEQ ID NO:26, SEQ ID NO:30, SEQ ID NO:34, SEQ ID NO:38, SEQ ID NO:42, SEQ ID NO:46, SEQ ID NO:50, SEQ ID NO:54, SEQ ID NO:58, SEQ ID NO:62, and SEQ ID NO:66. In some embodiments, the antibody comprises a heavy chain variable domain having at least 90%, at least 91%, at least 92%, at least 93%, at least 94%, at least 95%, at least 96%, at least 97%, at least 98%, or at least 99% sequence identity to an amino acid sequence selected from the group consisting of SEQ ID NO: 4, SEQ ID NO: 8, SEQ ID NO:12, SEQ ID NO:16, SEQ ID NO:20, SEQ ID NO:24, SEQ ID NO:28, SEQ ID NO:32, SEQ ID NO:36, SEQ ID NO:40, SEQ ID NO:44, SEQ ID NO:48, SEQ ID NO:52, SEQ ID NO:56, SEQ ID NO:60, SEQ ID NO:64, SEQ ID NO:132, SEQ ID NO:134, SEQ ID NO:136, SEQ ID NO:138, SEQ ID NO:140, SEQ ID NO:142, SEQ ID NO:144, and SEQ ID NO:146, and the antibody further comprises a light chain variable domain having at least 90%, at least 91%, at least 92%, at least 93%, at least 94%, at least 95%, at least 96%, at least 97%, at least 98%, or at least 99% sequence identity to an amino acid sequence selected from the group consisting of SEQ ID NO: 6, SEQ ID NO: 10, SEQ ID NO:14, SEQ ID NO:18, SEQ ID NO:22, SEQ ID NO:26, SEQ ID NO:30, SEQ ID NO:34, SEQ ID NO:38, SEQ ID NO:42, SEQ ID NO:46, SEQ ID NO:50, SEQ ID NO:54, SEQ ID NO:58, SEQ ID NO:62, and SEQ ID NO:66.

In some embodiments, the invention provides an antibody that is a variant of any of the above antibodies having one or more amino acid substitutions, deletions, insertions or modifications, and which retains a biological function of the antibody. In some embodiments, the invention provides an antibody that binds to C16orf54 expressed on the cell surface and inhibits the growth of the cell. In some embodiments, the anti-C16orf54 antibody binds to C16orf54 expressed on the cell surface and inhibits cell proliferation. In some embodiments, the anti-C16orf54 antibody binds to C16orf54 expressed on the cell surface and induces cell death. In some embodiments, the anti-C16orf54 antibody binds to C16orf54 expressed on the cell surface and induces cell differentiation or de-differentiation. In some embodiments, the anti-C16orf54 antibody binds to C16orf54 expressed on the cell surface and induces cell activation. In some embodiments, the invention provides an antibody that is a variant of any one of the above antibodies having improvements in one or more of a property such as binding affinity, specificity, thermostability, expression level, effector function, glycosylation, reduced immunogenicity, or solubility as compared to the unmodified antibody.

In some embodiments, the invention provides any one of the above antibodies or functional fragments, wherein the antibody or fragment is conjugated to a cytotoxic agent. In various embodiments, the cytotoxic agent is selected from a chemotherapeutic agent, a drug, a growth inhibitory agent, a toxin, or a radioactive isotope. In some embodiments, the invention provides any one of the above antibodies or functional fragments, wherein the antibody or fragment is conjugated to a detectable marker. In various embodiments, the detectable marker is selected from a radioisotope, a metal chelator, an enzyme, a fluorescent compound, a bioluminescent compound and a chemiluminescent compound.

In an embodiment, the invention provides a hybridoma that produces a monoclonal antibody of the invention. In an embodiment, the invention provides a transgenic animal that produces a monoclonal antibody of the invention.

In some embodiments, a polynucleotide encoding any of the above antibodies is provided. In an embodiment, a vector comprising the polynucleotide is provided. In an embodiment, a host cell comprising the vector is provided. In an embodiment, the host cell is prokaryotic. In an embodiment, the host cell is an E. coli cell. In another embodiment, the host cell is eukaryotic. In an embodiment, the host cell is a Chinese Hamster Ovary (CHO) cell. In an embodiment, a method of making an anti-C16orf54 antibody is provided, wherein the method comprises culturing the host cell under conditions suitable for expression of the polynucleotide encoding the antibody, and isolating the antibody.

In an embodiment, the invention provides a method of inhibiting growth of cancer cells that express C16orf54, the method comprising exposing the cells to any of the above antibodies or functional fragments, or an antibody conjugate of the invention. In some embodiments, the cancer cells are from a cancer selected from chronic lymphocytic leukemia, acute lymphocytic leukemia, acute myeloid leukemia, chronic myeloid leukemia, a lymphoma, a myeloma, breast cancer and pancreatic cancer.

In one embodiment, the invention provides a pharmaceutical composition comprising any of the above antibodies or functional fragments thereof, antibody conjugates, or binding agents of the invention. In a further embodiment, the invention provides a method of inhibiting growth of cancer cells that express C16orf54, the method comprising exposing the cells to any one or more of the above antibodies or functional fragments thereof, antibody conjugates, or binding agents of the invention. In various embodiments, the cancer cells are from a hematologic cancer, including but not limited to a leukemia (e.g., CLL, ALL, AML, CML), a lymphoma or a myeloma, and solid tumors such as breast cancer and pancreatic cancer or metastases of any of these cancers.

In an embodiment, the invention provides a method for treating a cancer in a subject comprising administering to the subject a pharmaceutical composition comprising any of the above antibodies or functional fragments thereof, antibody conjugates, or binding agents of the invention. In various embodiments, the cancer is selected from a hematologic cancer, including but not limited to a leukemia (e.g., CLL, ALL, AML, CML), a lymphoma or a myeloma, and solid tumors such as breast cancer and pancreatic cancer or metastases of any of these cancers. In some embodiments, the cancer is associated with increased expression of C16orf54 on the surface of a cell. In some embodiments, the antibody conjugates are antibody-drug conjugates (ADCs) comprising an antibody that binds to C16orf54 (e.g., the extracellular domain of C16orf54), for example, an ADC of the formula A-L-CTX, wherein A is an antibody, L is a linker, and CTX is a cytotoxic agent. In some embodiments, the method for treating a cancer comprises administering to a therapeutically effective amount of an anti-C16orf54 antibody or antibody-drug conjugate comprising an anti-C16orf54 antibody.

In some embodiments, the subject is administered one or more chemotherapeutic compound in combination with the antibody or functional fragment, wherein the chemotherapeutic compound is selected from chlorambucil, bendamustine hydrochloride, clophosphamide, fludurabine, pentostatin, cladribine, nelarabine, cytarabine, prednisone, prednisolone, methylprednisolone, dexamethasone, melphalan, lenalidomide, thalidomide, flavopiridol, oblimersen, ABT-263, doxorubicin, daunorubicin, idarubicin, mitoxentrone, methotrexate, clofarabine, imatinib mesylate, bosutinib, dasatinib, nilotinib, bortezomib, azacytidine, decitabine, midostaurin, sorafenib, AC220, arsenic trioxide, all-trans retinoic acid, vincristine sulfate, rituximab, ofatumumab, obinutuzumab, veltuzumab, ocrelizumab, lumiliximab, alemtuzumab and gemtuzumab ozogamicin.

In some embodiments, the one or more chemotherapeutic compound is selected from chlorambucil, bendamustine hydrochloride, clophosphamide, fludurabine, pentostatin, cladribine, prednisone, prednisolone, lenalidomide, flavopiridol, oblimersen, ABT-263, rituximab, ofatumumab, obinutuzumab, veltuzumab, ocrelizumab, lumiliximab, and alemtuzumab. In other embodiments, the one or more chemotherapeutic compound is selected from cytarabine, lenalidomide, doxorubicin, daunorubicin, idarubicin, mitoxentrone, clofarabine, azacytidine, decitabine, midostaurin, sorafenib, AC220, arsenic trioxide, all-trans retinoic acid, vincristine sulfate, and gemtuzumab ozogamicin.

In an embodiment, a method of detecting the presence of C16orf54 in a biological sample is provided, the method comprising contacting the biological sample with any of the above antibodies or functional fragments thereof, antibody conjugates, or binding agents of the invention under conditions permissive for binding of the antibody to C16orf54, and detecting whether a complex is formed between the antibody and C16orf54. In some embodiments, the biological sample is from a mammal having or suspected of having a cancer of cells or tissues including, but not limited to, a leukemia (e.g., CLL, ALL, AML, CML), a lymphoma, a myeloma, and solid tumors such as breast cancer and pancreatic cancer, or metastases of any of these cancers.

In an embodiment, a method of diagnosing a cancer associated with increased expression of C16orf54 is provided, the method comprising contacting a test cell with any of the above antibodies or functional fragments thereof, antibody conjugates, or binding agents of the invention; determining the level of expression of C16orf54 by detecting binding of the antibody or functional fragments thereof, antibody conjugates, or binding agents of the invention to C16orf54; and comparing the level of expression of C16orf54 by the test cell with the level of expression of C16orf54 by a control cell, wherein a higher level of expression of C16orf54 by the test cell as compared to the control cell indicates the presence of a cancer associated with increased expression of C16orf54. In some embodiments, the test cell is a cell from a patient suspected of having a cancer selected from a leukemia, a lymphoma, a myeloma, and a solid tumor such as breast cancer or pancreatic cancer, or a metastasis of any of these cancers.

In an embodiment, a method of killing a tumor cell is provided, the method comprising contacting a tumor cell expressing C16orf54 with an amount of an anti-C16orf54 antibody or antibody-drug conjugate comprising an anti-C16orf54 antibody effective to kill the tumor cell. In some embodiments, the tumor cell is from a hematologic cancer, including but not limited to a leukemia (e.g., CLL, ALL, AML, CML), a lymphoma or a myeloma, and solid tumors such as a breast cancer and pancreatic cancer or a tumor cell from metastases of any of these cancers. In some embodiments, the antibody conjugates are antibody-drug conjugates (ADCs) comprising an antibody that binds to C16orf54 (e.g., the extracellular domain of C16orf54), for example, an ADC of the formula A-L-CTX, wherein A is an antibody, L is a linker, and CTX is a cytotoxic agent.

In an embodiment, the method comprises determining the level of expression of C16orf54 on the surface of the test cell and comparing the level of expression of C16orf54 on the surface of the test cell with the level of expression of C16orf54 on the surface of the control cell. In some embodiments, the test cell is a cancer cell and the control cell is a normal cell of the same tissue type. In some embodiments, the test cell is a leukemia cell and the control cell is a bone marrow mononuclear cell or a peripheral blood mononuclear cell.

In an embodiment, the invention provides a use of any of the above antibodies or functional fragments thereof, antibody conjugates, or binding agents of the invention in the in the manufacture of a medicament, wherein the medicament is for use in a method of inhibiting growth of cancer cells that express C16orf54, the method comprising exposing the cells to the antibody or functional fragment thereof, antibody conjugate, or binding agent of the invention. In some embodiments, the cancer cells are from a cancer selected from chronic lymphocytic leukemia, acute lymphocytic leukemia, acute myeloid leukemia, chronic myeloid leukemia, a lymphoma, a myeloma, breast cancer and pancreatic cancer.

In an embodiment, the invention provides any of the above antibodies or functional fragments thereof, antibody conjugates, or binding agents of the invention for use in inhibiting the growth of cancer cells that express C16orf54. In some embodiments, the cancer cells are from a cancer selected from chronic lymphocytic leukemia, acute lymphocytic leukemia, acute myeloid leukemia, chronic myeloid leukemia, a lymphoma, a myeloma, breast cancer and pancreatic cancer.

In an embodiment, the invention provides a use of a pharmaceutical composition comprising any of the above antibodies or functional fragments thereof, antibody conjugates, or binding agents of the invention in the manufacture of a medicament, wherein the medicament is for use in a method of treating cancer in a subject, the method comprising administering the pharmaceutical composition to the subject. In various embodiments, the cancer is selected from a hematologic cancer, including but not limited to a leukemia (e.g., CLL, ALL, AML, CML), a lymphoma or a myeloma, and solid tumors such as breast cancer and pancreatic cancer or metastases of any of these cancers. In some embodiments, the cancer is associated with increased expression of C16orf54 on the surface of a cell. In some embodiments, the subject is administered one or more chemotherapeutic compound in combination with the antibody or functional fragment, wherein the chemotherapeutic compound is selected from chlorambucil, bendamustine hydrochloride, clophosphamide, fludurabine, pentostatin, cladribine, nelarabine, cytarabine, prednisone, prednisolone, methylprednisolone, dexamethasone, melphalan, lenalidomide, thalidomide, flavopiridol, oblimersen, ABT-263, doxorubicin, daunorubicin, idarubicin, mitoxentrone, methotrexate, clofarabine, imatinib mesylate, bosutinib, dasatinib, nilotinib, bortezomib, azacytidine, decitabine, midostaurin, sorafenib, AC220, arsenic trioxide, all-trans retinoic acid, vincristine sulfate, rituximab, ofatumumab, obinutuzumab, veltuzumab, ocrelizumab, lumiliximab, alemtuzumab and gemtuzumab ozogamicin. In some embodiments, the one or more chemotherapeutic compound is selected from chlorambucil, bendamustine hydrochloride, clophosphamide, fludurabine, pentostatin, cladribine, prednisone, prednisolone, lenalidomide, flavopiridol, oblimersen, ABT-263, rituximab, ofatumumab, obinutuzumab, veltuzumab, ocrelizumab, lumiliximab, and alemtuzumab. In other embodiments, the one or more chemotherapeutic compound is selected from cytarabine, lenalidomide, doxorubicin, daunorubicin, idarubicin, mitoxentrone, clofarabine, azacytidine, decitabine, midostaurin, sorafenib, AC220, arsenic trioxide, all-trans retinoic acid, vincristine sulfate, and gemtuzumab ozogamicin.

In an embodiment, the invention provides a pharmaceutical composition comprising any of the above antibodies or functional fragments thereof, antibody conjugates, or binding agents of the invention and a pharmaceutically acceptable carrier, for use in treating cancer in a subject. In various embodiments, the cancer is selected from a hematologic cancer, including but not limited to a leukemia (e.g., CLL, ALL, AML, CML), a lymphoma or a myeloma, and solid tumors such as breast cancer and pancreatic cancer or metastases of any of these cancers. In some embodiments, the cancer is associated with increased expression of C16orf54 on the surface of a cell. In some embodiments, the subject is administered one or more chemotherapeutic compound in combination with the antibody or functional fragment, wherein the chemotherapeutic compound is selected from chlorambucil, bendamustine hydrochloride, clophosphamide, fludurabine, pentostatin, cladribine, nelarabine, cytarabine, prednisone, prednisolone, methylprednisolone, dexamethasone, melphalan, lenalidomide, thalidomide, flavopiridol, oblimersen, ABT-263, doxorubicin, daunorubicin, idarubicin, mitoxentrone, methotrexate, clofarabine, imatinib mesylate, bosutinib, dasatinib, nilotinib, bortezomib, azacytidine, decitabine, midostaurin, sorafenib, AC220, arsenic trioxide, all-trans retinoic acid, vincristine sulfate, rituximab, ofatumumab, obinutuzumab, veltuzumab, ocrelizumab, lumiliximab, alemtuzumab and gemtuzumab ozogamicin. In some embodiments, the one or more chemotherapeutic compound is selected from chlorambucil, bendamustine hydrochloride, clophosphamide, fludurabine, pentostatin, cladribine, prednisone, prednisolone, lenalidomide, flavopiridol, oblimersen, ABT-263, rituximab, ofatumumab, obinutuzumab, veltuzumab, ocrelizumab, lumiliximab, and alemtuzumab. In other embodiments, the one or more chemotherapeutic compound is selected from cytarabine, lenalidomide, doxorubicin, daunorubicin, idarubicin, mitoxentrone, clofarabine, azacytidine, decitabine, midostaurin, sorafenib, AC220, arsenic trioxide, all-trans retinoic acid, vincristine sulfate, and gemtuzumab ozogamicin.

In an embodiment, the invention provides the use of any of the above antibodies or functional fragments thereof, antibody conjugates, or binding agents of the invention in the manufacture of a medicament, wherein the medicament is for use in a method for detecting the presence of of C16orf54 in a biological sample, the method comprising contacting the biological sample with the antibody or functional fragment thereof, antibody conjugate, or binding agent under conditions permissive for binding of the antibody to C16orf54, and detecting whether a complex is formed between the antibody or functional fragment thereof, antibody conjugate, or binding agent and C16orf54. In some embodiments, the biological sample is from a mammal having or suspected of having a cancer of cells or tissues including, but not limited to, a leukemia (e.g., CLL, ALL, AML, CML), a lymphoma, a myeloma, and solid tumors such as breast cancer and pancreatic cancer, or metastases of any of these cancers.

In an embodiment, the invention provides any of the above antibodies or functional fragments thereof, antibody conjugates, or binding agents of the invention for use in a method of detecting the presence of C16orf54 in a biological sample, the method comprising contacting the biological sample with the antibody functional fragment thereof, antibody conjugate, or binding agent under conditions permissive for binding of the antibody or functional fragment thereof, antibody conjugate, or binding agent to C16orf54, and detecting whether a complex is formed between the antibody or functional fragment thereof, antibody conjugate, or binding agent and C16orf54. In some embodiments, the biological sample is from a mammal having or suspected of having a cancer of cells or tissues including, but not limited to, a leukemia (e.g., CLL, ALL, AML, CML), a lymphoma, a myeloma, and solid tumors such as breast cancer and pancreatic cancer, or metastases of any of these cancers.

In an embodiment, the invention provides the use of any of the above antibodies or functional fragments thereof, antibody conjugates, or binding agents of the invention in the manufacture of a medicament, wherein the medicament is for use in a method of diagnosing a cancer associated with increased expression of C16orf54, the method comprising determining the level of expression of C16orf54 by detecting binding of the antibody or functional fragment, antibody conjugate, or binding agent of the invention to C16orf54; and comparing the level of expression of C16orf54 in the test cell with the level of expression of C16orf54 in a control cell, wherein a higher level of expression of C16orf54 in the test cell as compared to the control cell indicates the presence of a cancer associated with increased expression of C16orf54. In some embodiments, the test cell is a cell from a patient suspected of having a cancer selected from a leukemia, a lymphoma, a myeloma, and a solid tumor such as breast cancer or pancreatic cancer, or a metastasis of any of these cancers. In an embodiment, the method comprises determining the level of expression of C16orf54 on the surface of the test cell and comparing the level of expression of C16orf54 on the surface of the test cell with the level of expression of C16orf54 on the surface of the control cell. In some embodiments, the test cell is a cancer cell and the control cell is a normal cell of the same tissue type. In some embodiments, the test cell is a leukemia cell and the control cell is a bone marrow mononuclear cell or a peripheral blood mononuclear cell.

In an embodiment, the invention provides any of the above antibodies or functional fragments thereof, antibody conjugates, or binding agents of the invention for use in a method of diagnosing a cancer associated with increased expression of C16orf54, the method comprising determining the level of expression of C16orf54 by detecting binding of the antibody or functional fragment thereof, antibody conjugate, or binding agent of the invention to C16orf54; and comparing the level of expression of C16orf54 in the test cell with the level of expression of C16orf54 in a control cell, wherein a higher level of expression of C16orf54 in the test cell as compared to the control cell indicates the presence of a cancer associated with increased expression of C16orf54. In some embodiments, the test cell is a cell from a patient suspected of having a cancer selected from a leukemia, a lymphoma, a myeloma, and a solid tumor such as breast cancer or pancreatic cancer, or a metastasis of any of these cancers. In an embodiment, the method comprises determining the level of expression of C16orf54 on the surface of the test cell and comparing the level of expression of C16orf54 on the surface of the test cell with the level of expression of C16orf54 on the surface of the control cell. In some embodiments, the test cell is a cancer cell and the control cell is a normal cell of the same tissue type. In some embodiments, the test cell is a leukemia cell and the control cell is a bone marrow mononuclear cell or a peripheral blood mononuclear cell.

In another embodiment of the invention, an article of manufacture, or “kit”, containing materials useful for the treatment of the disorders described above is provided. The article of manufacture comprises a container and a label or package insert on or associated with the container. Suitable containers include, for example, bottles, vials, syringes, blister pack, etc. The containers may be formed from a variety of materials such as glass or plastic. The container holds an antibody or an antibody-drug conjugate (ADC) composition which is effective for treating the condition, and may have a sterile access port (for example the container may be an intravenous solution bag or a vial having a stopper pierceable by a hypodermic injection needle). At least one active agent in the composition is an antibdy or ADC. The label or package insert indicates that the composition is used for treating the condition of choice, such as cancer. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### BRIEF DESCRIPTION OF THE FIGURES

FIG. 1 shows the protein expression levels of CD19, CD20, and C16orf54 as identified and quantified by sTAg analysis in CLL specimens and relevant normal controls. Lines indicate the mean of % normalized spectral abundance factor (NSAF) in positive samples. Expression of CD19, CD20 and C16orf54 protein in 33 CLL patient samples, 11 normal PBMC and 11 normal BMMC samples.

FIG. 2 shows the results of competition ELISA to establish competitive binding bins for anti-huC16orf54 monoclonal antibodies. (A) Heatmap of 132 IgG2a antibodies binned against 25 IgG1/2b antibodies. (B) Clustergram of 132 IgG2a antibodies.

FIG. 3 shows relative binding properties of anti-huC16orf54 antibodies R29-7-1C, R29-67-1B, R29-67-3C, R29-67-4A, R29-67-5A, R29-67-7A, R29-67-9A, and R29-7-2A derived using a competition ELISA.

FIG. 4 shows an EC50 ELISA for the anti-huC16orf54 monoclonal antibodies R29-7-2A, R29-7-1C, R29-67-4A, and R29-67-7A. An isotype control, R22-4-26, is also shown.

FIG. 5 shows the inhibition of in vivo tumor growth in an acute myeloid leukemia xenograft. Acute myeloid leukemia cell line KG-1 was used as a therapeutic xenograft efficacy model, which expresses C16rf54 on the cell surface. Tumor volumes at which treatment was initiated were 92 mm³. Anti C16orf54 monoclonal antibodies 7-1C and 67-7A induced a statistically significant tumor growth inhibition of 69% (p=0.0078) and 70% (p=0.0068), respectively. Anti C16orf54 monoclonal antibodies 67-4A and 7-2A did not induce any statistically significant tumor growth inhibition. HB121 was used as an IgG isotype negative control antibody.

FIG. 6A-64 shows a sequence alignment of the variable heavy chains and variable light chains of the anti-C16orf54 monoclonal antibodies designated R29-7-2A, R29-7-1C, R29-67-7A, R29-8-136C, R29-8-57B, R29-7-54C, R29-7-53A, R29-8-50C, R29-8-19B, R29-8-58C, R29-8-9B, R29-8-28C, R29-8-120B, R29-8-75B, R29-8-36C, R29-8-12A, R29-8-93B, R29-8-51B, R29-8-30A, R29-8-18B, R29-7-38C, R29-7-49A, R29-7-13A and R29-67-4A. Boundaries of CDR's are indicated by Kabat, AbM, Chothia, Contact and IMGT numbering.

### DETAILED DESCRIPTION OF EMBODIMENTS OF THE INVENTION

General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3rd. edition (2001) Cold Spring Harbor Laboratory Press, Cold Spring Harbor, N.Y.; Current Protocols in Molecular Biology (F. M. Ausubel, et al. eds., (2003)); Therapeutic Monoclonal Antibodies: From Bench to Clinic, Z. An, ed, Wiley, Hoboken N.J. (2009); Monoclonal Antibodies: Methods and Protocols, M. Albitar, ed., Humana Press, Totawa, N.J. (2010); and Antibody Engineering, 2nd Ed., Vols 1 and 2, Kontermann and Dubel, eds., Springer-Verlag, Heidelberg, 2010.

### TERMINOLOGY

Unless defined otherwise, all technical and scientific terms used herein have the same meaning as is commonly understood by one of ordinary skill in the art. For purposes of interpreting this specification, the following definitions will apply and whenever appropriate, terms used in the singular will also include the plural and vice versa. All patents, applications, published applications and other publications are incorporated by reference in their entirety. In the event that any definition set forth conflicts with any document incorporated herein by reference, the definition set forth below shall control.

The term “C16orf54” or “C16orf54 polypeptide” and similar terms refers to the polypeptide (“polypeptide,” and “protein” are used interchangeably herein) or any native Chromosome 16 Open Reading Frame 54 (C16orf54) from any vertebrate source, including mammals such as primates (e.g., humans, cynomolgus monkey (cyno)), dogs, and rodents (e.g., mice and rats), unless otherwise indicated, and, in certain embodiments, included related C16orf54 polypeptides, including SNP variants thereof. The amino acid of human C16orf54 (“huC16orf54”) is provided below:

(SEQ ID NO: 1)

MPLTPEPPSGRVEGPPAWEAAPWPSLPCGPCIPIMLVLATLAALFILTTA
VLAERLFRRALRPDPSHRAPTLVWRPGGELWIEPMGTARERSEDWYGSAV
PLLTDRAPEPPTQVGTLEARATAPPAPSAPNSAPSNLGPQTVLEVPARST
FWGPQPWEGRPPATGLVSWAEPEQRPEASVQFGSPQARRQRPGSPDPEWG
LQPRVTLEQISAFWKREGRTSVGF.

The encoding nucleic acid sequence of huC16orf54 is provided below:

(SEQ ID NO: 2)

ATGCCGTTGACTCCAGAGCCGCCCTCTGGGCGCGTGGAGGGGCCCCCC
GCATGGGAAGCAGCCCCATGGCCCTCACTGCCCTGTGGGCCCTGCATCCC
CATCATGCTGGTCCTGGCCACCCTGGCTGCGCTCTTCATCCTCACCACCG
CTGTGTTGGCTGAACGCCTGTTCCGCCGTGCTCTCCGCCCAGACCCCAGC
CACCGTGCACCCACCCTGGTGTGGCGCCCAGGAGGAGAGCTGTGGATTGA
GCCCATGGGCACCGCCCGAGAGCGCTCTGAGGACTGGTATGGCTCTGCGG
TCCCCCTGCTGACAGATCGGGCCCCTGAGCCTCCCACCCAGGTGGGCACT
TTGGAGGCCCGAGCAACAGCCCCACCTGCCCCCTCAGCCCCAAATTCTGC
TCCCAGCAACTTGGGCCCCCAGACCGTACTGGAGGTCCCAGCCCGGAGCA
CCTTCTGGGGGCCCCAGCCCTGGGAGGGGAGGCCCCCCGCCACAGGCCTG
GTGAGCTGGGCTGAACCCGAGCAGAGGCCAGAGGCCAGCGTCCAGTTTGG
GAGCCCCCAGGCCAGGAGGCAGCGGCCAGGGAGCCCGGATCCTGAGTGGG
GCCTCCAGCCACGGGTCACCTTGGAGCAGATCTCAGCTTTCTGGAAGCGT
GAAGGCCGGACCAGTGTGGGGTTCTGA.

The amino acid sequence of the predicted transmembrane protein C16orf54 for cynomolgus monkey (cyno), scientific name Macaca fascicularis, is provided below:

(SEQ ID NO: 318)

MPSTPEPPSGRMEGPPTWEAAPWPSLPCGPCIPIMLALATLAALFILTTA
VLAERLFRRALRPDPSHHAPTLVWRPGGELWIEPMGTPRERSEDWYGSAV
PLLTDRAPEPPTPVGVLEAQATAPYAPSAPNSAPSSLGPQTPPEVPARST
FWGPQPWEGRPPGTGLVSWAEPEQRPEARAQFGSPQARRQRPRSPDPEWG
LQPRVTLEQISAFWRREGRTSVGF

The amino acid sequence of the transmembrane protein C16orf54 homolog for mouse, scientific name Mus musculus, is provided below:

(SEQ ID NO: 319)

MPVTPQQPSGHTEGLPEPTAEAAVWVVIPCGPCIPIMLGLASLTAFFIIT
TAVLAERLFRRPQPDPSQRAPTLVWRPGGELWIEPTSSARERSEDWYGSS
MPLLMDRAPGPPTPGGTLEGRATAPPATSAPYSSLSSLVPQTPPEVPAQS
TFWRPQTQEERPHDTSLVSWVGSEPMPEAGLQVGSPRPWRPRQGSLEPDW
GLQPRVTLEQISAFWKREGRTSVGF

Related polypeptides include allelic variants (e.g., SNP variants); splice variants; fragments; derivatives; substitution, deletion, and insertion variants; fusion polypeptides; and interspecies homologs, preferably, which retain C16orf54 activity and/or are sufficient to generate an anti-C16orf54 immune response. As those skilled in the art will appreciate, an anti-C16orf54 antibody provided herein can bind to a C16orf54 polypeptide, polypeptide fragment, antigen, and/or epitope, as an epitope is part of the larger antigen, which is part of the larger polypeptide fragment, which, in turn, is part of the larger polypeptide. C16orf54 can exist in a native or denatured form. The C16ORF54 polypeptides described herein may be isolated from a variety of sources, such as from human tissue types or from another source, or prepared by recombinant or synthetic methods. A “native sequence C16ORF54 polypeptide” comprises a polypeptide having the same amino acid sequence as the corresponding C16ORF54 polypeptide derived from nature. Such native sequence C16ORF54 polypeptides can be isolated from nature or can be produced by recombinant or synthetic means. The term “native sequence C16ORF54 polypeptide” specifically encompasses naturally-occurring truncated or secreted forms of the specific C16ORF54 polypeptide (e.g., an extracellular domain sequence), naturally-occurring variant forms (e.g., alternatively spliced forms) and naturally-occurring allelic variants of the polypeptide. Orthologs to the C16orf54 polypeptide are also well known in the art.

The term “C16orf54” encompasses “full-length,” unprocessed C16orf54 as well as any form of C16orf54 that results from processing in the cell. The term also encompasses naturally occurring variants or mutations of C16orf54, e.g., splice variants, allelic variants, SNP variants and isoforms. The C16orf54 polypeptides described herein may be isolated from a variety of sources, such as from human tissue types or from another source, or prepared by recombinant or synthetic methods. A “native sequence C16orf54 polypeptide” comprises a polypeptide having the same amino acid sequence as the corresponding C16orf54 polypeptide derived from nature. Such native sequence C16orf54 polypeptides can be isolated from nature or can be produced by recombinant or synthetic means. The term “native sequence C16orf54 polypeptide” specifically encompasses naturally-occurring truncated or secreted forms of the specific C16orf54 polypeptide (e.g., an extracellular domain sequence), naturally-occurring variant forms (e.g., alternatively spliced forms) and naturally-occurring allelic variants of the polypeptide.

The term “antibody” and “immunoglobulin” or “Ig” are used interchangeably herein, and is used in the broadest sense and specifically covers, for example, single anti-C16orf54 monoclonal antibodies (including agonist, antagonist, neutralizing antibodies, full length or intact monoclonal antibodies), anti-C16orf54 antibody compositions with polyepitopic specificity, polyclonal antibodies, multivalent antibodies, multispecific antibodies (e.g., bispecific antibodies so long as they exhibit the desired biological activity), formed from at least two intact antibodies, single chain anti-C16orf54 antibodies, and fragments of anti-C16orf54 antibodies, as defined below. An antibody can be human, humanized, chimeric and/or affinity matured as well as an antibody from other species, e.g., mouse, rabbit etc. The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa) and each amino-terminal portion of each chain includes a variable region of about 100 to about 130 or more amino acids and each carboxy-terminal portion of each chain includes a constant region (See, Borrebaeck (ed.) (1995) Antibody Engineering, Second Ed., Oxford University Press.; Kuby (1997) Immunology, Third Ed., W.H. Freeman and Company, New York). In specific embodiments, the specific molecular antigen can be bound by an antibody provided herein includes the target C16orf54 polypeptide, fragment or epitope.

Antibodies also include, but are not limited to, synthetic antibodies, monoclonal antibodies, recombinantly produced antibodies, multispecific antibodies (including bi-specific antibodies), human antibodies, humanized antibodies, camelized antibodies, chimeric antibodies, intrabodies, anti-idiotypic (anti-Id) antibodies, and functional fragments of any of the above, which refers a portion of an antibody heavy or light chain polypeptide that retains some or all of the binding activity of the antibody from which the fragment was derived. Non-limiting examples of functional fragements include single-chain Fvs (scFv) (e.g., including monospecific, bispecific, etc.), Fab fragments, F(ab′) fragments, F(ab)₂ fragements, F(ab′)₂ fragments, disulfide-linked Fvs (sdFv), Fd fragments, Fv fragments, diabody, triabody, tetrabody and minibody. In particular, antibodies provided herein include immunoglobulin molecules and immunologically active portions of immunoglobulin molecules, e.g., antigen binding domains or molecules that contain an antigen-binding site that binds to a C16orf54 antigen (e.g., one or more complementarity determining regions (CDRs) of an anti-C16orf54 antibody). Such antibody fragments can be found described in, for example, Harlow and Lane, Antibodies: A Laboratory Manual, Cold Spring Harbor Laboratory, New York (1989); Myers (ed.), Molec. Biology and Biotechnology: A Comprehensive Desk Reference, New York: VCH Publisher, Inc.; Huston et al., Cell Biophysics, 22:189-224 (1993); Plückthun and Skerra, Meth. Enzymol., 178:497-515 (1989) and in Day, E. D., Advanced Immunochemistry, Second Ed., Wiley-Liss, Inc., New York, N.Y. (1990). The antibodies provided herein can be of any type (e.g., IgG, IgE, IgM, IgD, IgA and IgY), any class (e.g., IgG1, IgG2, IgG3, IgG4, IgA1 and IgA2), or any subclass (e.g., IgG2a and IgG2b) of immunoglobulin molecule. An anti-C16orf54 antibodies provided herein can be agonistic antibodies or antagonistic antibodies.

An “antigen” is a predetermined antigen to which an antibody can selectively bind. The target antigen may be a polypeptide, carbohydrate, nucleic acid, lipid, hapten or other naturally occurring or synthetic compound. Preferably, the target antigen is a polypeptide.

The term “antigen binding fragment,” “antigen binding domain,” “antigen binding region,” and similar terms refer to that portion of an antibody which comprises the amino acid residues that interact with an antigen and confer on the binding agent its specificity and affinity for the antigen (e.g., the complementarity determining regions (CDRs)).

The terms “binds” or “binding” as used herein refer to an interaction between molecules to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions or forces. The strength of the total non-covalent interactions between a single antigen-binding site on an antibody and a single epitope of a target molecule, such as C16orf54, is the affinity of the antibody or functional fragment for that epitope. The ratio of association (k₁) to dissociation (k₋₁) of an antibody to a monovalent antigen (k₁/k₋₁) is the association constant K, which is a measure of affinity. The value of K varies for different complexes of antibody and antigen and depends on both k₁ and k₋₁. The association constant K for an antibody provided herein can be determined using any method provided herein or any other method well known to those skilled in the art. The affinity at one binding site does not always reflect the true strength of the interaction between an antibody and an antigen. When complex antigens containing multiple, repeating antigenic determinants, such as a polyvalent C16orf54, come in contact with antibodies containing multiple binding sites, the interaction of antibody with antigen at one site will increase the probability of a reaction at a second site. The strength of such multiple interactions between a multivalent antibody and antigen is called the avidity. The avidity of an antibody can be a better measure of its binding capacity than is the affinity of its individual binding sites. For example, high avidity can compensate for low affinity as is sometimes found for pentameric IgM antibodies, which can have a lower affinity than IgG, but the high avidity of IgM, resulting from its multivalence, enables it to bind antigen effectively.

The terms “antibodies that specifically bind to C16orf54,” “antibodies that specifically bind to a C16orf54 epitope,” “anti-C16orf54 antibodies” and analogous terms are also used interchangeably herein and refer to antibodies that specifically bind to a C16orf54 polypeptide, such as a C16orf54 antigen or epitope. An antibody that specifically binds to C16orf54 may bind to the extracellular domain or peptide derived from the extracellular domain of C16orf54. An antibody that specifically binds to a C16orf54 antigen may be cross-reactive with related antigens. In certain embodiments, an antibody that specifically binds to a C16orf54 antigen does not cross-react with other antigens. An antibody that specifically binds to a C16orf54 antigen can be identified, for example, by immunoassays, BIAcore, or other techniques known to those of skill in the art. An antibody binds specifically to a C16orf54 antigen when it binds to a C16orf54 antigen with higher affinity than to any cross-reactive antigen as determined using experimental techniques, such as radioimmunoassays (RIA) and enzyme-linked immunosorbent assays (ELISAs). Typically a specific or selective reaction will be at least twice background signal or noise and more typically more than 10 times background. See, e.g., Paul, ed., 1989, Fundamental Immunology Second Edition, Raven Press, New York at pages 332-336 for a discussion regarding antibody specificity. An antibody “which binds” an antigen of interest is one that binds the antigen with sufficient affinity such that the antibody is useful as a therapeutic agent in targeting a cell or tissue expressing the antigen, and does not significantly cross-react with other proteins. In such embodiments, the extent of binding of the antibody to a “non-target” protein will be less than about 10% of the binding of the antibody to its particular target protein as determined by fluorescence activated cell sorting (FACS) analysis or radioimmunoprecipitation (RIA). With regard to the binding of an antibody to a target molecule, the term “specific binding” or “specifically binds to” or is “specific for” a particular polypeptide or an epitope on a particular polypeptide target means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding” or “specifically binds to” or is “specific for” a particular polypeptide or an epitope on a particular polypeptide target as used herein can be exhibited, for example, by a molecule having a Kd for the target of at least about 10⁻⁴ M, alternatively at least about 10⁻⁵ M, alternatively at least about 10⁻⁶ M, alternatively at least about 10⁻⁷ M, alternatively at least about 10⁻⁸ M, alternatively at least about 10⁻⁹ M, alternatively at least about 10⁻¹⁰ M, alternatively at least about 10⁻¹¹ M, alternatively at least about 10⁻¹² M, or greater. In one embodiment, the term “specific binding” refers to binding where a molecule binds to a particular polypeptide or epitope on a particular polypeptide without substantially binding to any other polypeptide or polypeptide epitope. In certain embodiments, an antibody that binds to C16orf54 has a dissociation constant (Kd) of ≦1 μM, ≦100 nM, ≦10 nM, ≦1 nM, or ≦0.1 nM. In certain embodiments, anti-C16orf54 antibody binds to an epitope of C16orf54 that is conserved among C16orf54 from different species.

The term “anti-C16orf54 antibody” or “an antibody that binds to C16orf54” refers to an antibody that is capable of binding C16orf54 with sufficient affinity such that the antibody is useful as a diagnostic and/or therapeutic agent in targeting C16orf54. Preferably, the extent of binding of an anti-C16orf54 antibody to an unrelated, non-C16orf54 protein is less than about 10% of the binding of the antibody to C16orf54 as measured, e.g., by fluorescence activated cell sorting (FACS) analysis or a radioimmunoassay (RIA). An antibody that “specifically binds to” or is “specific for” C16orf54 is defined as above. In certain embodiments, an antibody that binds to C16orf54 has a dissociation constant (Kd) of ≦1 μM, ≦100 nM, ≦10 nM, ≦1 nM, or ≦0.1 nM. In certain embodiments, anti-C16orf54 antibody binds to an epitope of C16orf54 that is conserved among C16orf54 from different species.

An “isolated” antibody is substantially free of cellular material or other contaminating proteins from the cell or tissue source and/or other contaminant components from which the antibody is derived, or substantially free of chemical precursors or other chemicals when chemically synthesized. The language “substantially free of cellular material” includes preparations of an antibody in which the antibody is separated from cellular components of the cells from which it is isolated or recombinantly produced. Thus, an antibody that is substantially free of cellular material includes preparations of antibody having less than about 30%, 20%, 10%, or 5% (by dry weight) of heterologous protein (also referred to herein as a “contaminating protein”). In certain embodiments, when the antibody is recombinantly produced, it is substantially free of culture medium, e.g., culture medium represents less than about 20%, 10%, or 5% of the volume of the protein preparation. In certain embodiments, when the antibody is produced by chemical synthesis, it is substantially free of chemical precursors or other chemicals, e.g., it is separated from chemical precursors or other chemicals which are involved in the synthesis of the protein. Accordingly such preparations of the antibody have less than about 30%, 20%, 10%, 5% (by dry weight) of chemical precursors or compounds other than the antibody of interest. Contaminant components can also include, but are not limited to, materials that would interfere with therapeutic uses for the antibody, and may include enzymes, hormones, and other proteinaceous or nonproteinaceous solutes. In certain embodiments, the antibody will be purified (1) to greater than 95% by weight of antibody as determined by the Lowry method (Lowry et al. J. Bio. Chem. 193: 265-275, 1951), such as 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step. In a specific embodiment, antibodies provided herein are isolated

The basic 4-chain antibody unit is a heterotetrameric glycoprotein composed of two identical light (L) chains and two identical heavy (H) chains. In the case of IgGs, the 4-chain unit is generally about 150,000 daltons. Each L chain is linked to a H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain (C_{L}) at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71 and Chapter 6.

The term “variable domain” or “variable region” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable domain of the heavy chain may be referred to as “VH.” The variable domain of the light chain may be referred to as “VL.” The term “variable” refers to the fact that certain segments of the variable domains differ extensively in sequence among antibodies. The V domain mediates antigen binding and defines specificity of a particular antibody for its particular antigen. However, the variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md., 1991)). The constant domains are not involved directly in binding an antibody to an antigen, but exhibit various effector functions, such as participation of the antibody in antibody dependent cellular cytotoxicity (ADCC) and complement dependent cytotoxicity (CDC). The variable domains differ extensively in sequence between different antibodies. The variability in sequence is concentrated in the CDRs while the less variable portions in the variable domain are referred to as framework regions (FR). The CDRs of the light and heavy chains are primarily responsible for the interaction of the antibody with antigen. In specific embodiments, the variable region is a human variable region.

The term “variable domain residue numbering as in Kabat” or “amino acid position numbering as in Kabat”, and variations thereof, refers to the numbering system used for heavy chain variable domains or light chain variable domains of the compilation of antibodies in Kabat et al., Sequences of Proteins of Immunological Interest, 5th Ed. Public Health Service, National Institutes of Health, Bethesda, Md. (1991). Using this numbering system, the actual linear amino acid sequence may contain fewer or additional amino acids corresponding to a shortening of, or insertion into, a FR or CDR of the variable domain. For example, a heavy chain variable domain may include a single amino acid insert (residue 52a according to Kabat) after residue 52 of H2 and inserted residues (e.g. residues 82a, 82b, and 82c, etc, according to Kabat) after heavy chain FR residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g, Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG 1 EU antibody. Unless stated otherwise herein, references to residue numbers in the variable domain of antibodies means residue numbering by the Kabat numbering system. Unless stated otherwise herein, references to residue numbers in the constant domain of antibodies means residue numbering by the EU numbering system.

An “intact” antibody is one comprising an antigen-binding site as well as a CL and at least heavy chain constant domains, C_{H}1, C_{H}2 and C_{H}3. The constant domains may be native sequence constant domains (e.g., human native sequence constant domains) or amino acid sequence variant thereof. Preferably, the intact antibody has one or more effector functions.

“Antibody fragments” comprise a portion of an intact antibody, preferably the antigen binding or variable region of the intact antibody. Examples of antibody fragments include, without limitation, Fab, Fab′, F(ab′)₂, and Fv fragments; diabodies and di-diabodies (see, e.g., Holliger, P. et al. (1993) Proc. Natl. Acad. Sci. 90:6444-8; Lu, D. et al. (2005) J. Biol. Chem. 280:19665-72; Hudson et al., Nat. Med. 9:129-134 (2003); WO 93/11161; and U.S. Pat. Nos. 5,837,242 and 6,492,123); single-chain antibody molecules (see, e.g., U.S. Pat. Nos. 4,946,778; 5,260,203; 5,482,858 and 5,476,786); dual variable domain antibodies (see, e.g., U.S. Pat. No. 7,612,181); single variable domain antibodies (SdAbs) (see, e.g., Woolven et al., Immunogenetics 50: 98-101, 1999; Streltsov et al., Proc Natl Acad Sci USA. 101:12444-12449, 2004); and multispecific antibodies formed from antibody fragments.

A “functional fragment” of a therapeutic antibody will exhibit at least one if not some or all of the biological functions attributed to the intact antibody, the function comprising at least specific binding to the target antigen.

The term “fusion protein” as used herein refers to a polypeptide that comprises an amino acid sequence of an antibody and an amino acid sequence of a heterologous polypeptide or protein (e.g., a polypeptide or protein not normally a part of the antibody (e.g., a non-anti-C16orf54 antigen antibody)). The term “fusion” when used in relation to C16orf54 or to an anti-C16orf54 antibody refers to the joining of a peptide or polypeptide, or fragment, variant and/or derivative thereof, with a heterologous peptide or polypeptide. In certain embodiments, the fusion protein retains the biological activity of the C16orf54 or anti-C16orf54 antibody. In certain embodiments, the fusion protein comprises a C16orf54 antibody VH domain, VL domain, VH CDR (one, two or three VH CDRs), and/or VL CDR (one, two or three VL CDRs), wherein the fusion protein binds to a C16orf54 epitope.

The term “heavy chain” when used in reference to an antibody refers to a polypeptide chain of about 50-70 kDa, wherein the amino-terminal portion includes a variable region of about 120 to 130 or more amino acids and a carboxy-terminal portion that includes a constant region. The constant region can be one of five distinct types, referred to as alpha (α), delta (δ), epsilon (ε), gamma (γ) and mu (μ), based on the amino acid sequence of the heavy chain constant region. The distinct heavy chains differ in size: α, δ and γ contain approximately 450 amino acids, while p and c contain approximately 550 amino acids. When combined with a light chain, these distinct types of heavy chains give rise to five well known classes of antibodies, IgA, IgD, IgE, IgG and IgM, respectively, including four subclasses of IgG, namely IgG1, IgG2, IgG3 and IgG4. A heavy chain can be a human heavy chain.

The term “host” as used herein refers to an animal, such as a mammal (e.g., a human).

The term “host cell” as used herein refers to the particular subject cell transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

The term “monoclonal antibody” as used herein refers to an antibody obtained from a population of substantially homogeneous antibodies, e.g., the individual antibodies comprising the population are identical except for possible naturally occurring mutations that may be present in minor amounts, and each monoclonal antibody will typically recognize a single epitope on the antigen. In specific embodiments, a “monoclonal antibody,” as used herein, is an antibody produced by a single hybridoma or other cell, wherein the antibody binds to only a C16orf54 epitope as determined, e.g., by ELISA or other antigen-binding or competitive binding assay known in the art. The term “monoclonal” is not limited to any particular method for making the antibody. For example, the monoclonal antibodies useful in the present invention may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example. Other methods for the preparation of clonal cell lines and of monoclonal antibodies expressed thereby are well known in the art (see, for example, Chapter 11 in: Short Protocols in Molecular Biology, (2002) 5th Ed., Ausubel et al., eds., John Wiley and Sons, New York). Other exemplary methods of producing other monoclonal antibodies are provided in the Examples herein.

The term “native” when used in connection with biological materials such as nucleic acid molecules, polypeptides, host cells, and the like, refers to those which are found in nature and not manipulated by a human being.

The antibodies provided herein can include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., Proc. Natl. Acad. Sci. USA, 81:6851-6855 (1984)).

“Humanized” forms of nonhuman (e.g., murine) antibodies are chimeric antibodies that include human immunoglobulins (recipient antibody) in which the native CDR residues are replaced by residues from the corresponding CDR of a nonhuman species (donor antibody) such as mouse, rat, rabbit or nonhuman primate having the desired specificity, affinity, and capacity. In some instances, one or more FR region residues of the human immunoglobulin are replaced by corresponding nonhuman residues. Furthermore, humanized antibodies can comprise residues that are not found in the recipient antibody or in the donor antibody. These modifications are made to further refine antibody performance. A humanized antibody heavy or light chain can comprise substantially all of at least one or more variable domains, in which all or substantially all of the CDRs correspond to those of a nonhuman immunoglobulin and all or substantially all of the FRs are those of a human immunoglobulin sequence. In certain embodiments, the humanized antibody will comprise at least a portion of an immunoglobulin constant region (Fc), typically that of a human immunoglobulin. For further details, see, Jones et al., Nature, 321:522-525 (1986); Riechmann et al., Nature, 332:323-329 (1988); and Presta, Curr. Op. Struct. Biol., 2:593-596 (1992); Carter et al., Proc. Natl. Acd. Sci. USA 89:4285-4289 (1992); and U.S. Pat. No. 6,800,738 (issued Oct. 5, 2004), U.S. Pat. No. 6,719,971 (issued Sep. 27, 2005), U.S. Pat. No. 6,639,055 (issued Oct. 28, 2003), U.S. Pat. No. 6,407,213 (issued Jun. 18, 2002), and U.S. Pat. No. 6,054,297 (issued Apr. 25, 2000).

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries (Hoogenboom and Winter, J. Mol. Biol., 227:381 (1991); Marks et al., J. Mol. Biol., 222:581 (1991) and yeast display libraries (Chao et al., Nature Protocols 1: 755-768 (2006)). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., mice (see, e.g., Jakobovits, A., Curr. Opin. Biotechnol. 1995, 6(5):561-6; Brüggemann and Taussing, Curr. Opin. Biotechnol. 1997, 8(4):455-8; and U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

A “CDR” refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody VL β-sheet framework. Accordingly, CDRs are variable region sequences interspersed within the framework region sequences. CDR regions are well known to those skilled in the art and have been defined by, for example, Kabat as the regions of most hypervariability within the antibody variable (V) domains (Kabat et al., J. Biol. Chem. 252:6609-6616 (1977); Kabat, Adv. Prot. Chem. 32:1-75 (1978)). CDR region sequences also have been defined structurally by Chothia as those residues that are not part of the conserved β-sheet framework, and thus are able to adapt different conformations (Chothia and Lesk, J. Mol. Biol. 196:901-917 (1987)). Both terminologies are well recognized in the art. The positions of CDRs within a canonical antibody variable domain have been determined by comparison of numerous structures (Al-Lazikani et al., J. Mol. Biol. 273:927-948 (1997); Morea et al., Methods 20:267-279 (2000)). Because the number of residues within a hypervariable region varies in different antibodies, additional residues relative to the canonical positions are conventionally numbered with a, b, c and so forth next to the residue number in the canonical variable domain numbering scheme (Al-Lazikani et al., supra (1997)). Such nomenclature is similarly well known to those skilled in the art.

The term “hypervariable region”, “HVR”, or “HV”, when used herein refers to the regions of an antibody variable domain that are hypervariable in sequence and/or form structurally defined loops. Generally, antibodies comprise six hypervariable regions; three in the VH (H1, H2, H3), and three in the VL (L1, L2, L3). A number of hypervariable region delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software. The “contact” hypervariable regions are based on an analysis of the available complex crystal structures. The residues from each of these hypervariable regions are noted below.

Recently, a universal numbering system has been developed and widely adopted, ImMunoGeneTics (IMGT) Information System® (Lafranc et al., Dev. Comp. Immunol. 27(1):55-77 (2003)). IMGT is an integrated information system specializing in immunoglobulins (IG), T cell receptors (TR) and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues and are readily identified. This information can be used in grafting and replacement of CDR residues from immunoglobulins of one species into an acceptor framework from, typically, a human antibody. Correspondence between the Kabat numbering and the IMGT unique numbering system is also well known to one skilled in the art (e.g. Lefranc et al., supra).

| Loop | IMGT | Kabat | AbM | Chothia | Contact |
|---|---|---|---|---|---|
| — | — | — | — | — | — |
| L1 | 27-38 | L24-L34 | L24-L34 | L26-L32 | L30-L36 |
| L2 | 56-65 | L50-L56 | L50-L56 | L50-L52 | L46-L55 |
| L3 | 105-117 | L89-L97 | L89-L97 | L91-L96 | L89-L96 |
| H1 | 27-38 | H31-H35B | H26-H35B | H26-H32 | H30-H35B |

(Kabat Numbering)

| | | | | | |
|---|---|---|---|---|---|
| H1 | 27-38 | H31-H35 | H26-H35 | H26-H32 | H30-H35 |

(Chothia Numbering)

| | | | | | |
|---|---|---|---|---|---|
| H2 | 56-65 | H50-H65 | H50-H58 | H53-H55 | H47-H58 |
| H3 | 105-117 | H95-H102 | H95-H102 | H96-H101 | H93-H101 |

Hypervariable regions may comprise “extended hypervariable regions” as follows: 24-36 or 24-34 (L1), 46-56 or 50-56 (L2) and 89-97 or 89-96 (L3) in the VL and 26-35 or 26-35A (H1), 50-65 or 49-65 (H2) and 93-102, 94-102, or 95-102 (H3) in the VH. The variable domain residues are numbered according to Kabat et al., supra, for each of these definitions. As used herein, the terms “HVR” and “CDR” are used interchangeably.

The term “constant region” or “constant domain” refers to a carboxy terminal portion of the light and heavy chain which is not directly involved in binding of the antibody to antigen but exhibits various effector function, such as interaction with the Fc receptor. The terms refer to the portion of an immunoglobulin molecule having a more conserved amino acid sequence relative to the other portion of the immunoglobulin, the variable domain, which contains the antigen binding site. The constant domain contains the CH1, CH2 and CH3 domains of the heavy chain and the CL domain of the light chain.

The term “framework” or “FR” residues are those variable domain residues flanking the CDRs. FR residues are present, e.g., in chimeric, humanized, human, domain antibodies, diabodies, linear antibodies, and bispecific antibodies. FR residues are those variable domain residues other than the hypervariable region residues herein defined.

The term “variable domain residue numbering as in Kabat” or “amino acid position numbering as in Kabat”, and variations thereof, refers to the numbering system used for heavy chain variable domains or light chain variable domains of the compilation of antibodies in Kabat et al., Sequences of Proteins of Immunological Interest, 5th Ed. Public Health Service, National Institutes of Health, Bethesda, Md. (1991). Using this numbering system, the actual linear amino acid sequence may contain fewer or additional amino acids corresponding to a shortening of, or insertion into, a FR or CDR of the variable domain. For example, a heavy chain variable domain may include a single amino acid insert (residue 52a according to Kabat) after residue 52 of H2 and inserted residues (e.g., residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-113 of the heavy chain) (e.g., Kabat et al., Sequences of Immunological Interest, 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody. Unless stated otherwise herein, references to residue numbers in the variable domain of antibodies means residue numbering by the Kabat numbering system. Unless stated otherwise herein, references to residue numbers in the constant domain of antibodies means residue numbering by the EU numbering system.

An “affinity matured” antibody is one with one or more alterations in one or more HVRs thereof which result in an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art. For review, see Hudson and Souriau, Nature Medicine 9:129-134 (2003); Hoogenboom, Nature Biotechnol. 23: 1105-1116 (2005); Quiroz and Sinclair, Revista Ingeneria Biomedia 4: 39-51 (2010).

A “blocking” antibody or an “antagonist” antibody is one which inhibits or reduces biological activity of the antigen it binds. Preferred blocking antibodies or antagonist antibodies substantially or completely inhibit the biological activity of the antigen.

An “agonist antibody”, as used herein, is an antibody that triggers a response, e.g., one that mimics at least one of the functional activities of a polypeptide of interest.

An “agonist” of C16orf54 refers to a molecule that is capable of activating or otherwise increasing one or more of the biological activities of C16orf54, such as in a cell expressing C16orf54 or in a cell expressing a C16orf54 ligand, such as a C16orf54 receptor. In some embodiments, an agonist of C16orf54 (e.g., an agonistic antibody provided herein) may, for example, act by activating or otherwise increasing the activation and/or cell signaling pathways of the cell expressing a C16orf54 or a C16orf54 receptor, thereby increasing a C16orf54-mediated biological activity of the cell the relative to the C16orf54-mediated biological activity in the absence of agonist. In certain embodiments the antibodies provided herein are agonistic anti-C16orf54 antibodies.

As used herein, an “antagonist” or “inhibitor” of C16orf54 refers to a molecule that is capable of inhibiting or otherwise decreasing one or more of the biological activities of C16orf54, such as in a cell expressing C16orf54 or in a cell expressing a C16orf54 ligand, such as a C16orf54 receptor. In some embodiments, an antagonist of C16orf54 (e.g., an antagonistic antibody provided herein) may, for example, act by inhibiting or otherwise decreasing the activation and/or cell signaling pathways of the cell expressing a C16orf54 or a C16orf54 receptor, thereby inhibiting a C16orf54-mediated biological activity of the cell the relative to the C16orf54-mediated biological activity in the absence of antagonist. In certain embodiments the antibodies provided herein are antagonistic anti-C16orf54 antibodies.

“Binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention. Specific illustrative embodiments are described in the following.

In one embodiment, the “Kd” or “Kd value” according to this invention is measured by a radiolabeled antigen binding assay (RIA) performed with the Fab version of an antibody of interest and its antigen as described by the following assay that measures solution binding affinity of Fabs for antigen by equilibrating Fab with a minimal concentration of (¹²⁵I)-labeled antigen in the presence of a titration series of unlabeled antigen, then capturing bound antigen with an anti-Fab antibody-coated plate (Chen, et al., (1999) J. Mol Biol 293:865-881). According to another embodiment the Kd or Kd value is measured by using surface plasmon resonance assays using, for example, a BIAcore™-2000 or a BIAcore™-3000 (BIAcore, Inc., Piscataway, N.J.), or by biolayer interferometry using, for example, the OctetQK384 sytem (ForteBio, Menlo Park, Calif.).

An “on-rate” or “rate of association” or “association rate” or “kₒₙ” according to this invention can also be determined with the same surface plasmon resonance or biolayer interferometry techniques described above using, for example, a BIAcore™-2000 or a BIAcore™-3000 (BIAcore, Inc., Piscataway, N.J.), or the OctetQK384 sytem (ForteBio, Menlo Park, Calif.).

The phrase “substantially similar,” or “substantially the same”, as used herein, denotes a sufficiently high degree of similarity between two numeric values (generally one associated with an antibody of the invention and the other associated with a reference antibody) such that one of skill in the art would consider the difference between the two values to be of little or no biological and/or statistical significance within the context of the biological characteristic measured by said values (e.g., Kd values). The difference between the two values is preferably less than about 50%, preferably less than about 40%, preferably less than about 30%, preferably less than about 20%, preferably less than about 10% as a function of the value for the reference antibody.

The phrase “substantially reduced,” or “substantially different”, as used herein, denotes a sufficiently high degree of difference between two numeric values (generally one associated with an antibody of the invention and the other associated with a reference antibody) such that one of skill in the art would consider the difference between the two values to be of statistical significance within the context of the biological characteristic measured by said values (e.g., Kd values, HAMA response). The difference between said two values is preferably greater than about 10%, preferably greater than about 20%, preferably greater than about 30%, preferably greater than about 40%, preferably greater than about 50% as a function of the value for the reference antibody.

An antibody that “inhibits the growth of cells expressing a C16orf54 polypeptide” or a “growth inhibitory” antibody is one that results in measurable growth inhibition of cells expressing or overexpressing the appropriate C16orf54 polypeptide. In certain embodiments, the cells are tumor cells or cancer cells as emplified herein, but other types of cells are contemplated. The C16orf54 polypeptide may be a transmembrane polypeptide expressed on the surface of a cancer cell or may be a polypeptide that is produced and secreted by a cancer cell. Preferred growth inhibitory anti-C16orf54 antibodies inhibit growth of C16orf54-expressing tumor cells by greater than 20%, preferably from about 20% to about 50%, and even more preferably, by greater than 50% (e.g., from about 50% to about 100%) as compared to the appropriate control, the control typically being tumor cells not treated with the antibody being tested. In one embodiment, growth inhibition can be measured at an antibody concentration of about 0.1 to 30 μg/ml or about 0.5 nM to 200 nM in cell culture, where the growth inhibition is determined 1-10 days after exposure of the tumor cells to the antibody. Growth inhibition of tumor cells in vivo can be determined in various ways such as is described below. The antibody is growth inhibitory in vivo if administration of the anti-C16orf54 antibody at about 1 μg/kg to about 100 mg/kg body weight results in reduction in tumor size or tumor cell proliferation within about 5 days to 3 months from the first administration of the antibody, preferably within about 5 to 30 days.

An antibody that “induces apoptosis” is one that induces programmed cell death as determined by binding of annexin V, fragmentation of DNA, cell shrinkage, dilation of endoplasmic reticulum, cell fragmentation, and/or formation of membrane vesicles (called apoptotic bodies). The cell is usually one that overexpresses a C16orf54 polypeptide. Preferably the cell is a tumor cell. Various methods are available for evaluating the cellular events associated with apoptosis. For example, phosphatidyl serine (PS) translocation can be measured by annexin binding; DNA fragmentation can be evaluated through DNA laddering; and nuclear/chromatin condensation along with DNA fragmentation can be evaluated by any increase in hypodiploid cells. Preferably, the antibody which induces apoptosis is one which results in about 2 to 50 fold, preferably about 5 to 50 fold, and most preferably about 10 to 50 fold, induction of annexin binding relative to untreated cell in an annexin binding assay.

An antibody that “induces cell death” is one that causes a viable cell to become nonviable. The cell is of a cell type that specifically expresses or overexpresses a C16orf54 polypeptide. The cell may be cancerous or a normal cell of the particular cell type. The C16orf54 polypeptide may be a transmembrane polypeptide expressed on the surface of a cancer cell or may be a polypeptide that is produced and secreted by a cancer cell. Cell death in vitro may be determined in the absence of complement and immune effector cells to distinguish cell death induced by antibody-dependent cell-mediated cytotoxicity (ADCC) or complement dependent cytotoxicity (CDC). Thus, the assay for cell death may be performed using heat inactivated serum (e.g., in the absence of complement) and in the absence of immune effector cells. One way to determine whether the antibody is able to induce cell death, is to assess loss of membrane integrity as evaluated by uptake of propidium iodide (PI), trypan blue (see Moore et al. Cytotechnology 17:1-11 (1995)) or 7AAD relative to untreated cells. In some embodiments, cell death-inducing antibodies are those which induce PI uptake in the PI uptake assay in C16orf54 expressing cells.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptor); and B cell activation.

The term “Fc region” herein is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue.

A “functional Fc region” possesses an “effector function” of a native sequence Fc region. Exemplary “effector functions” include C1q binding; CDC; Fc receptor binding; ADCC; phagocytosis; down regulation of cell surface receptors (e.g., B cell receptor; BCR), etc. Such effector functions generally require the Fc region to be combined with a binding domain (e.g., an antibody variable domain) and can be assessed using various assays as disclosed, for example, in definitions herein.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g., from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

The term “variant” when used in relation to C16orf54 or to an anti-C16orf54 antibody refers to a peptide or polypeptide comprising one or more (such as, for example, about 1 to about 25, about 1 to about 20, about 1 to about 15, about 1 to about 10, or about 1 to about 5) amino acid sequence substitutions, deletions, and/or additions as compared to a native or unmodified sequence. For example, a C16orf54 variant may result from one or more (such as, for example, about 1 to about 25, about 1 to about 20, about 1 to about 15, about 1 to about 10, or about 1 to about 5) changes to an amino acid sequence of native C16orf54. Also by way of example, a variant of an anti-C16orf54 antibody may result from one or more (such as, for example, about 1 to about 25, about 1 to about 20, about 1 to about 15, about 1 to about 10, or about 1 to about 5) changes to an amino acid sequence of a native or previously unmodified anti-C16orf54 antibody. Variants may be naturally occurring, such as allelic or splice variants, or may be artificially constructed. Polypeptide variants may be prepared from the corresponding nucleic acid molecules encoding the variants. In specific embodiments, the C16orf54 variant or anti-C16orf54 antibody variant at least retains C16orf54 or anti-C16orf54 antibody functional activity, respectively. In specific embodiments, an anti-C16orf54 antibody variant binds C16orf54 and/or is antagonistic to C16orf54 activity. In specific embodiments, an anti-C16orf54 antibody variant binds C16orf54 and/or is agonistic to C16orf54 activity. In certain embodiments, the variant is encoded by a single nucleotide polymorphism (SNP) variant of a nucleic acid molecule that encodes C16orf54 or anti-C16orf54 antibody VH or VL regions or subregions.

The term “vector” refers to a substance that is used to introduce a nucleic acid molecule into a host cell. Vectors applicable for use include, for example, expression vectors, plasmids, phage vectors, viral vectors, episomes and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more nucleic acid molecules are to be co-expressed (e.g. both an antibody heavy and light chain), both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the nucleic acid molecule is expressed in a sufficient amount to produce the desired product (e.g. an anti-C16orf54 antibody provided herein), and it is further understood that expression levels can be optimized to obtain sufficient expression using methods well known in the art.

“Antibody-dependent cell-mediated cytotoxicity” or “ADCC” refers to a form of cytotoxicity in which secreted Ig bound onto Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. (USA) 95:652-656 (1998).

“Fc receptor” or “FcR” describes a receptor that binds to the Fc region of an antibody. The preferred FcR is a native sequence human FcR. Moreover, a preferred FcR is one that binds an IgG antibody (a gamma receptor) and includes receptors of the FcγRI, FcγRII and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof (see review M. in Daëron, Annu. Rev. Immunol. 15:203-234 (1997)). FcRs are reviewed in Ravetch and Kinet, Annu. Rev. Immunol. 9:457-492 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)). Antibody variants with improved or diminished binding to FcRs are described, for example, in WO 2000/42072, and U.S. Pat. Nos. 7,183,387; 7,332,581; and 7,335,742. See also, e.g., Shields et al. J. Biol. Chem. 9(2):6591-6604 (2001).

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay, e.g., as described in Gazzano-Santoro et al., J. Immunol. Methods 202:163 (1996), may be performed. Polypeptide variants with altered Fc region amino acid sequences (polypeptides with a variant Fc region) and increased or decreased C1q binding capability are described, e.g., in U.S. Pat. No. 6,194,551 B1 and WO 1999/51642. See also, e.g., Idusogie et al. J. Immunol. 164: 4178-4184 (2000).

The C16orf54 polypeptide “extracellular domain” or “ECD” refers to a form of the C16orf54 polypeptide that is essentially free of the transmembrane and cytoplasmic domains. Ordinarily, a C16orf54 polypeptide ECD will have less than 1% of such transmembrane and/or cytoplasmic domains and preferably, will have less than 0.5% of such domains. The transmembrane domain of C16orf54 comprises amino acid residues from about 32 to about 53. The exact boundaries of a transmembrane domain may vary but most likely by no more than about 5 amino acids at either end of the domain as initially identified. Optionally, therefore, an extracellular domain of a C16orf54 polypeptide may comprise amino acids from about 1 to 27-37 of the sequence of C16orf54 as shown in SEQ ID NO:1.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

A “modification” of an amino acid residue/position, as used herein, refers to a change of a primary amino acid sequence as compared to a starting amino acid sequence, wherein the change results from a sequence alteration involving said amino acid residue/positions. For example, typical modifications include substitution of the residue with another amino acid (e.g., a conservative or non-conservative substitution), insertion of one or more (generally fewer than 5 or 3) amino acids adjacent to said residue/position, and deletion of said residue/position.

An “epitope” is the site on the surface of an antigen molecule to which a single antibody molecule binds, such as a localized region on the surface of an antigen, such as C16orf54 polypeptide or C16orf54 polypeptide fragment, that is capable of being bound to one or more antigen binding regions of an antibody, and that has antigenic or immunogenic activity in an animal, such as a mammal (e.g., a human), that is capable of eliciting an immune response. An epitope having immunogenic activity is a portion of a polypeptide that elicits an antibody response in an animal. An epitope having antigenic activity is a portion of a polypeptide to which an antibody binds as determined by any method well known in the art, for example, by an immunoassay. Antigenic epitopes need not necessarily be immunogenic. Epitopes usually consist of chemically active surface groupings of molecules such as amino acids or sugar side chains and have specific three dimensional structural characteristics as well as specific charge characteristics. The term specifically includes linear epitopes and conformational epitopes. A region of a polypeptide contributing to an epitope may be contiguous amino acids of the polypeptide or the epitope may come together from two or more non-contiguous regions of the polypeptide. The epitope may or may not be a three-dimensional surface feature of the antigen. In certain embodiments, a C16orf54 epitope is a three-dimensional surface feature of a C16orf54 polypeptide. In other embodiments, a C16orf54 epitope is linear feature of a C16orf54 polypeptide. Generally an antigen has several or many different epitopes and reacts with many different antibodies.

An antibody binds “essentially the same epitope” as a reference antibody, when the two antibodies recognize identical, overlapping epitopes or adjacent epitopes in a three-dimensional space. The most widely used and rapid methods for determining whether two antibodies bind to identical, overlapping epitopes or adjacent epitopes in a three-dimensional space are competition assays, which can be configured in a number of different formats, using either labeled antigen or labeled antibody. Usually, the antigen is immobilized on a 96-well plate, or expressed on a cell surface, and the ability of unlabeled antibodies to block the binding of labeled antibodies is measured using radioactive, fluorescent or enzyme labels.

“Epitope mapping” is the process of identifying the binding sites, or epitopes, of antibodies on their target antigens. Antibody epitopes may be linear epitopes or conformational epitopes. Linear epitopes are formed by a continuous sequence of amino acids in a protein. Conformational epitopes are formed of amino acids that are discontinuous in the protein sequence, but which are brought together upon folding of the protein into its three-dimensional structure. Induced epitopes are formed when the three dimensional structure of the protein is in an altered confirmation, such as following activation or binding of another protein or ligand.

“Epitope binning”, as defined herein, is the process of grouping antibodies based on the epitopes they recognize. More particularly, epitope binning comprises methods and systems for discriminating the epitope recognition properties of different antibodies, using competition assays combined with computational processes for clustering antibodies based on their epitope recognition properties and identifying antibodies having distinct binding specificities.

A “C16orf54-expressing cell,” “a cell having expression of C16orf54” or a grammatical equivalent thereof refers to a cell that expresses endogenous or transfected C16orf54 on the cell surface. A cell expressing C16orf54 produces sufficient levels of C16orf54 on its surface, such that an anti-C16orf54 antibody can bind thereto. In some aspect, such binding may have a therapeutic effect with respect to the cancer. A cell that “overexpresses” C16orf54 is one that has significantly higher levels of C16orf54 at the cell surface thereof, compared to a cell of the same tissue type that is known to express C16orf54. Such overexpression may be caused by gene amplification or by increased transcription or translation. C16orf54 overexpression may be determined in a diagnostic or prognostic assay by evaluating increased levels of the C16orf54 protein present on the surface of a cell (e.g. via an immunohistochemistry assay; FACS analysis). Alternatively, or additionally, one may measure levels of C16orf54-encoding nucleic acid or mRNA in the cell, e.g. via fluorescent in situ hybridization; (FISH; see WO98/45479 published October, 1998), Southern blotting, Northern blotting, or polymerase chain reaction (PCR) techniques, such as real time quantitative PCR (RT-PCR). Aside from the above assays, various in vivo assays are available to the skilled practitioner. For example, one may expose cells within the body of the patient to an antibody which is optionally labeled with a detectable agent, and binding of the antibody to cells in the patient can be evaluated, e.g. by external scanning for radioactivity or by analyzing a biopsy taken from a patient previously exposed to the antibody. A C16orf54-expressing tumor cell includes, but is not limited to, acute myeloid leukemia (AML) tumor cells.

A “C16orf54-mediated disease” and “C16orf54-mediated disorder” are used interchangeably and refer to any disease that is completely or partially caused by or is the result of C16orf54. In certain embodiments, C16orf54 is aberrantly (e.g., highly) expressed on the surface of a cell. In some embodiments, C16orf54 may be aberrantly upregulated on a particular cell type. In other embodiments, normal, aberrant or excessive cell signaling is caused by binding of C16orf54 to a C16orf54 ligand, which can bind or otherwise interact with C16orf54.

A “disorder” is any condition or disease that would benefit from treatment with an substance/molecule or method of the invention. This includes chronic and acute disorders including those pathological conditions that predispose the mammal to the disorder in question. Non-limiting examples of disorders to be treated herein include cancerous conditions such as a leukemia (including, but not limited to CLL, ALL, AML, and CML), multiple myeloma, and certain solid tumors such as breast cancer and pancreatic cancer, or a metastasis of any of these cancers.

The terms “cell proliferative disorder” and “proliferative disorder” refer to disorders that are associated with some degree of abnormal cell proliferation. In one embodiment, the cell proliferative disorder is cancer. “Tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein. The terms “cancer” and “cancerous” refer to or describe the physiological condition in mammals that is typically characterized by unregulated cell growth. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, oral cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, multiple myeloma and B-cell lymphoma, brain cancer, as well as head and neck cancer, and associated metastases.

In some embodiments, a cancer may be a hematopoietic cancer, referring to cancers of the bone marrow and blood, and including both leukemia and myeloma. The term “myeloma” (also “multiple myeloma” or “plasma cell myeloma” refers to or describes a cancer of the plasma cells. The term “leukemia” refers to or describes any one of various acute or chronic neoplastic diseases of the blood-forming tissues characterized by distorted proliferation and development of leukocytes and their precursors in the blood and bone marrow. Leukemias are typically classified as either chronic (slowly progressing, and deriving from mature cells) or acute (rapidly progressing, and deriving from immature blasts). Leukemias are further classified based upon the type of white blood cell that is affected, either lymphoid cells (lymphoid, lymphocytic or lymphoblastic leukemia) or myeloid cells (myeloid, myelogenous, myeloblastic, or granulocytic leukemia). Examples of leukemia include but are not limited to acute leukemias, chronic leukemias, lymphoblastic leukemias, lymphocytic leukemias, myeloid leukemias, myelogenous leukemias, Acute lymphoblastic leukemia (ALL), Chronic lymphocytic leukemia (CLL), Acute myelogenous leukemia (AML), Chronic myelogenous leukemia (CML), Hairy cell leukemia (HCL), T-cell prolymphocytic leukemia (T-PLL), B-cell prolymphocytic leukemia (B-PLL), Large granular lymphocytic leukemia, MLL-positive leukemias and MLL-induced lukemias.

Chronic lymphocytic leukemia (CLL) is a chronic leukemia of the lymphoblastic type. The staging of CLL is based upon the Rai or Binet systems. The Rai system divides CLL into 5 stages:
Rai stage 0: The blood lymphocyte count is too high, usually defined as over 10,000 lymphocytes/mm3 of blood (this is called lymphocytosis). Some doctors will diagnose CLL if the count is over 5,000/mm3 and the cells all have the same chemical pattern on special testing. The lymph nodes, spleen, and liver are not enlarged and the red blood cell and platelet counts are near normal.
Rai stage I: Lymphocytosis plus enlarged lymph nodes. The spleen and liver are not enlarged and the red blood cell and platelet counts are near normal.
Rai stage II: Lymphocytosis plus an enlarged spleen (and possibly an enlarged liver), with or without enlarged lymph nodes. The red blood cell and platelet counts are near normal.
Rai stage III: Lymphocytosis plus anemia (too few red blood cells), with or without enlarged lymph nodes, spleen, or liver. Platelet counts are near normal.
Rai stage IV: Lymphocytosis plus thrombocytopenia (too few blood platelets), with or without anemia, enlarged lymph nodes, spleen, or liver.

Stage 0 is considered low risk, stages I and II considered intermediate risk, and stages III and IV are considered high risk.

In the Binet staging system, CLL is classified by the number of affected lymphoid tissue groups (neck lymph nodes, groin lymph nodes, underarm lymph nodes, spleen, and liver) and by whether or not the patient has anemia (too few red blood cells) or thrombocytopenia (too few blood platelets).
Binet stage A: Fewer than 3 areas of lymphoid tissue are enlarged, with no anemia or thrombocytopenia.
Binet stage B: 3 or more areas of lymphoid tissue are enlarged, with no anemia or thrombocytopenia.
Binet stage C: Anemia and/or thrombocytopenia are present.

The term “Chronic lymphocytic leukemia” or “CLL”, as used herein, may refer to any of these subtypes or stages.

Acute myeloid leukemia (AML) is an acute leukemia of the myeloblastic type.

The classification system from the World Health Organization (WHO) includes:
AML with recurrent genetic abnormalities (that is, with specific chromosomal changes)
AML with multilineage dysplasia (abnormalities in how the blood cells look)
AML, related to therapy that is damaging to cells (also called therapy-related myeloid neoplasm)
AML that is not otherwise categorized

The French-American-British (FAB) classification system (Bennett et al., 1976, Br J Haematol 33 (4): 451-458) classifies AML subtypes as follows:

M0: Myeloblastic without differentiation

M1: Myeloblastic without maturation

M2: Myeloblastic with maturation

M3: Promyelocytic

M4: Myelomonocytic

M5a: Monocytic without differentiation (monoblastic)

M5b: Monocytic with differentiation

M6: Erythroleukemic

M7: Megakaryocytic

The term “acute myeloid leukemia” or “AML”, as used herein, may refer to any of these subtypes.

A “C16orf54-expressing cell” is a cell that expresses endogenous or transfected C16orf54 on the cell surface. A “C16orf54-expressing cancer” is a cancer comprising cells that have C16orf54 protein present on the cell surface. A “C16orf54-expressing cancer” produces sufficient levels of C16orf54 on the surface of cells thereof, such that an anti-C16orf54 antibody can bind thereto and have a therapeutic effect with respect to the cancer. A cancer that “overexpresses” C16orf54 is one that has significantly higher levels of C16orf54 at the cell surface thereof, compared to a noncancerous cell of the same tissue type. Such overexpression may be caused by gene amplification or by increased transcription or translation or increased stability of the protein. C16orf54 overexpression may be determined in a diagnostic or prognostic assay by evaluating increased levels of the C16orf54 protein present on the surface of a cell (e.g., via an immunohistochemistry assay; FACS analysis). Alternatively, or additionally, one may measure levels of C16orf54-encoding nucleic acid or mRNA in the cell, e.g., via fluorescent in situ hybridization; (FISH; see WO98/45479 published October, 1998), Southern blotting, Northern blotting, or polymerase chain reaction (PCR) techniques, such as real time quantitative PCR (RT-PCR). Aside from the above assays, various in vivo assays are available to the skilled practitioner. For example, one may expose cells within the body of the patient to an antibody which is optionally labeled with a detectable label, e.g., a radioactive isotope, and binding of the antibody to cells in the patient can be evaluated, e.g., by external scanning for radioactivity or by analyzing a biopsy taken from a patient previously exposed to the antibody. A C16orf54-expressing cancer includes, but is not limited to, a leukemia, multiple myeloma, solid tumors such as breast cancer and pancreatic cancer, and metastases of any of these cancers.

As used herein, the terms “treat,” “treatment” and “treating” refer to the reduction or amelioration of the progression, severity, and/or duration of a C16orf54-mediated disease resulting from the administration of one or more therapies (including, but not limited to, the administration of one or more therapeutic agents, such as an antibody provided herein). “Treatment” (and variations such as “treat” or “treating”) also refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, preventing metastasis in the case of cancer, decreasing the rate of disease progression, amelioration or palliation of the disease state, and remission or improved prognosis. In some embodiments, antibodies of the invention are used to delay development of a disease or disorder or to slow the progression of a disease or disorder. In specific embodiments, such terms refer to the reduction or inhibition of cancer or tumor formation. In other specific embodiments, such term refers to the reduction or amelioration of the progression, severity and/or duration of graft-versus-host disease. In yet other specific embodiments, such terms refer to the reduction or amelioration of the progression, severity, and/or duration of a disease that is responsive to immune modulation, such modulation resulting from increasing T cell activation, increasing T cell proliferation or increasing cytokine production.

The above parameters for assessing successful treatment and improvement in the disease are readily measurable by routine procedures familiar to a physician. For cancer therapy, efficacy can be measured, for example, by assessing the time to disease progression (TTP) and/or by determining the response rate (RR). Other endpoints for measuring efficacy include, for example, overall survival (OS), disease-free survival (DFS) and recurrence-free (or relapse-free) survival (RFS). Metastasis can be determined by staging tests and by bone scan and tests for calcium level and other enzymes to determine spread to the bone. CT scans can also be done to look for spread to the pelvis and lymph nodes in the area. Chest X-rays and measurement of liver enzyme levels by known methods are used to look for metastasis to the lungs and liver, respectively. Other routine methods for monitoring the disease include transrectal ultrasonography (TRUS) and transrectal needle biopsy (TRNB).

An “individual” is a vertebrate. In certain embodiments, the vertebrate is a mammal. Mammals include, but are not limited to, farm animals (such as cows), sport animals, pets (such as cats, dogs, and horses), primates, mice and rats. In certain embodiments, a mammal is a human.

An “effective amount” is an amount sufficient to effect beneficial or desired results or to carry out a specifically state purpose, such as an amount effective, at dosages and for periods of time necessary, to achieve the desired therapeutic or prophylactic result. An “effective amount” may be determined empirically and in a routine manner, in relation to the stated purpose. An effective amount can be administered in one or more administrations, applications or dosages. Such delivery is dependent on a number of variables including the time period for which the individual dosage unit is to be used, the bioavailability of the agent, the route of administration, etc. In some embodiments, effective amount also refers to the amount of an antibody provided herein to achieve a specified result (e.g., inhibition of a C16orf54 biological activity of a cell, such as modulating T cell activation and/or proliferation). In some embodiments, this term refers to the amount of a therapy (e.g., an antibody or pharmaceutical composition provided herein) which is sufficient to reduce and/or ameliorate the severity and/or duration of a given disease and/or a symptom related thereto. This term also encompasses an amount necessary for the reduction or amelioration of the advancement or progression of a given disease, reduction or amelioration of the recurrence, development or onset of a given disease, and/or to improve or enhance the prophylactic or therapeutic effect(s) of another therapy (e.g., a therapy other than anti-C16orf54 antibody provided herein). In some embodiments, the effective amount of an antibody is from about 0.1 mg/kg (mg of antibody per kg weight of the subject) to about 100 mg/kg. In certain embodiments, an effective amount of an antibody provided therein is about 0.1 mg/kg, about 0.5 mg/kg, about 1 mg/kg, 3 mg/kg, 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg about 90 mg/kg or about 100 mg/kg (or a range therein).

The term “therapeutically effective amount” as used herein refers to the amount of a therapeutic agent (e.g., an antibody provided herein or any other therapeutic agent provided herein) that is sufficient to reduce and/or ameliorate the severity and/or duration of a given disease and/or a symptom related thereto. A therapeutically effective amount of a therapeutic agent can be an amount necessary for the reduction or amelioration of the advancement or progression of a given disease, reduction or amelioration of the recurrence, development or onset of a given disease, and/or to improve or enhance the prophylactic or therapeutic effect of another therapy (e.g., a therapy other than the administration of an antibody provided herein). A “therapeutically effective amount” of a substance/molecule of the invention may vary according to factors such as the disease state, age, sex, and weight of the individual, and the ability of the substance/molecule, to elicit a desired response in the individual. A therapeutically effective amount encompasses an amount in which any toxic or detrimental effects of the substance/molecule are outweighed by the therapeutically beneficial effects. In certain embodiments, the term “therapeutically effective amount” refers to an amount of an antibody or other drug effective to “treat” a disease or disorder in a subject or mammal. In the case of cancer, the therapeutically effective amount of the drug may reduce the number of cancer cells; reduce the tumor size; inhibit (e.g., slow to some extent and preferably stop) cancer cell infiltration into peripheral organs; inhibit (e.g., slow to some extent and preferably stop) tumor metastasis; inhibit, to some extent, tumor growth; and/or relieve to some extent one or more of the symptoms associated with the cancer. See the definition herein of “treating”. To the extent the drug may prevent growth and/or kill existing cancer cells, it may be cytostatic and/or cytotoxic.

A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount would be less than the therapeutically effective amount. In the case of cancer, the therapeutically effective amount of the drug may, for example, reduce the number of cancer cells; reduce the tumor size; inhibit (e.g., slow to some extent and preferably stop) cancer cell infiltration into peripheral organs; inhibit (e.g., slow to some extent and preferably stop) tumor metastasis; inhibit, to some extent, tumor growth; and/or relieve to some extent one or more of the symptoms associated with the cancer. See preceding definition of “treating”. To the extent the drug may prevent growth and/or kill existing cancer cells, it may be cytostatic and/or cytotoxic.

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

Administration “in combination with” one or more further therapeutic agents includes simultaneous (concurrent) and consecutive administration in any order. As used herein, the term “in combination” in the context of the administration of other therapies refers to the use of more than one therapy. The use of the term “in combination” does not restrict the order in which therapies are administered to a subject with an infection. A first therapy can be administered before (e.g., 1 minute, 45 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks), concurrently, or after (e.g., 1 minute, 45 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks) the administration of a second therapy to a subject which had, has, or is susceptible to a C16orf54-mediated disease. Any additional therapy can be administered in any order with the other additional therapies. In certain embodiments, the antibodies can be administered in combination with one or more therapies (e.g., therapies that are not the antibodies that are currently administered to prevent, treat, manage, and/or ameliorate a C16orf54-mediated disease. Non-limiting examples of therapies that can be administered in combination with an antibody include analgesic agents, anesthetic agents, antibiotics, or immunomodulatory agents or any other agent listed in the U.S. Pharmacopoeia and/or Physician's Desk Reference.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™. The term “carrier” can also refer to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a exemplary carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences (1990) Mack Publishing Co., Easton, Pa. Such compositions will contain a prophylactically or therapeutically effective amount of the antibody, e.g., in isolated or purified form, together with a suitable amount of carrier so as to provide the form for proper administration to the patient. The formulation should suit the mode of administration.

The term “pharmaceutically acceptable” as used herein means being approved by a regulatory agency of the Federal or a state government, or listed in the U.S. Pharmacopeia, European Pharmacopeia or other generally recognized Pharmacopeia for use in animals, and more particularly in humans.

The term “pharmaceutical formulation” refers to a preparation which is in such form as to permit the biological activity of the active ingredient to be effective, and which contains no additional components which are unacceptably toxic to a subject to which the formulation would be administered. Such formulation may be sterile.

A “sterile” formulation is aseptic of free from all living microorganisms and their spores.

“Polyclonal antibodies” as used herein refers to an antibody population generated in an immunogenic response to a protein having many epitopes and thus includes a variety of different antibodies directed to the same and to different epitopes within the protein. Methods for producing polyclonal antibodies are known in the art (See, e.g., see, for example, Chapter 11 in: Short Protocols in Molecular Biology, (2002) 5th Ed., Ausubel et al., eds., John Wiley and Sons, New York).

An “immunoconjugate” as used herein refers to an antibody that is conjugated to one or more cytotoxic agents (e.g., a chemotherapeutic agent, a drug, a growth inhibitory agent, a toxin, or a radioisotype) or diagnostic agents (e.g., a radioisotope, a metal chelator, an enzyme, a fluorescent compound, a bioluminescent compound, or a chemiluminescent compound). In some embodiments the antibody is covalently bound by a synthetic linker to the one or more cytotoxic or diagnostic agents. Immunoconjugates comprising antibodies conjugated to cytotoxic agents are also referred to herein as “antibody drug conjugates,” or “ADCs”. An “antibody-drug conjugate” or “ADC” is an antibody that is conjugated to one or more cytotoxic agents, for example, through one or more linkers. An ADC may be of the formula A-L-CTX, wherein A is an antibody, L is a linker, and CTX is a cytotoxic agent.

The term “cytotoxic agent” or “cytotoxin” or “CTX” as used herein refers to a substance that inhibits or prevents the function of cells and/or has a cytotoxic effect on cells (e.g., causes destruction of cells). The term is also intended to include alkylating agents, an anthracyclines, a cytoskeletal disruptors (taxanes), an epothilones, an histone deacetylase Inhibitor (HDAC), an inhibitor of Topoisomerase I, an Inhibitor of Topoisomerase II, a kinase inhibitor, a monoclonal antibodies, a nucleotide analog, a peptide antibiotic, a platinum-based agent, a retinoids, a Vinca alkaloid or a derivative thereof, and radioisotope. The term is also intended to include Actinomycin, all-trans retinoic acid, Azacitidine, Azathioprine, Bleomycin, Bortezomib, Carboplatin, Capecitabine, Cisplatin, Chlorambucil, Cyclophosphamide, Cytarabine, Daunorubicin, Docetaxel, Doxifluridine, Doxorubicin, Epirubicin, Epothilone, Etoposide, Fluorouracil, Gemcitabine, Hydroxyurea, Idarubicin, Imatinib, Irinotecan, Mechlorethamine, Mercaptopurine, Methotrexate, Mitoxantrone, Oxaliplatin, Paclitaxel, Pemetrexed, Teniposide, Tioguanine, Topotecan, Valrubicin, Vinblastine, Vincristine, Vindesine, and Vinorelbine. The term is also intended to include a tubulin stabilizer, a tubulin destabilizer, a DNA alkylator, a DNA minor groove binder, a DNA intercalator, a topoisomerase I inhibitor, a topoisomerase II inhibitor, a gyrase inhibitor, a protein synthesis inhibitor, a proteosome inhibitor, and an anti-metabolite. The term is also intended to include Actinomycin D, Amonafide, an auristatin, benzophenone, benzothiazole, a calicheamicin, Camptothecin, CC-1065 (NSC 298223), Cemadotin, Colchicine, Combretastatin A4, Dolastatin, Doxorubicin, Elinafide, Emtansine (DM1), Etoposide, KF-12347 (Leinamycin), a maytansinoid, Methotrexate, Mitoxantrone, Nocodazole, Proteosome Inhibitor 1 (PSI 1), Roridin A, T-2 Toxin (trichothecene analog), Taxol, a tubulysin, Velcade®, and Vincristine. Preferred cytotoxins include an auristatin, a calicheamicin, a maytansinoid, and a tubulysin. Other preferred cytotoxins include monomethylauristatin E, monomethylauristatin F, calicheamicin γ, mertansine, tubulysin T3, and tubulysin T4, the structures for which are provided below:

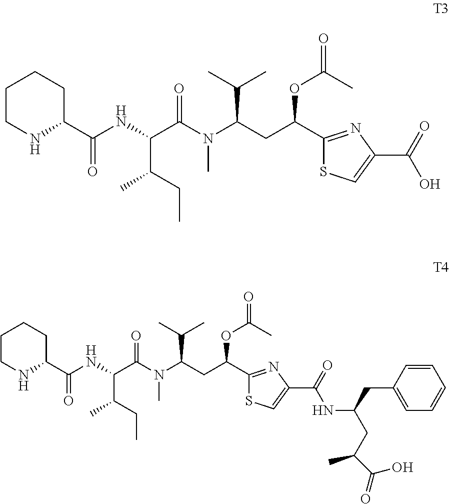 [SMILES: CC[C@H](C)[C@H](NC(=O)[C@H]1CCCCN1)C(=O)N(C)[C@H](C[C@@H](OC(C)=O)c1nc(C(=O)O)cs1)C(C)C ; CC[C@H](C)[C@H](NC(=O)[C@H]1CCCCN1)C(=O)N(C)[C@H](C[C@@H](OC(C)=O)c1nc(C(=O)N[C@@H](Cc2ccccc2)C[C@H](C)C(=O)O)cs1)C(C)C]

Other cytotoxic agents including various antitumor or anticancer agents are known in the art. The term is also intended to include radioactive isotopes (e.g., At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents e.g., methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including fragments and/or variants thereof, and the various antitumor or anticancer agents disclosed below. Other cytotoxic agents are described below. A tumoricidal agent causes destruction of tumor cells. A “toxin” is any substance capable of having a detrimental effect on the growth or proliferation of a cell.

A “chemotherapeutic agent” is a chemical agent (e.g., compound or drug) useful in the treatment of cancer, regardless of mechanism of action. Chemotherapeutic agents include compounds used in targeted therapy and conventional chemotherapy. Examples of chemotherapeutic agents include, but are not limited to, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem Intl. Ed. Engl., 33: 183-186 (1994)); dynemicin, including dynemicin A; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN®, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; gemcitabine (GEMZAR®); 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluoromethylornithine (DMFO); retinoids such as retinoic acid; capecitabine (XELODA®); pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin. Additional chemotherapeutic agents include cytotoxic agents useful as antibody drug conjugates, such as maytansinoids (DM1 and DM4, for example) and auristatins (MMAE and MMAF, for example).

Also included in the definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, 4-hydroxytamoxifen, trioxifene, keoxifene, LY 117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RI VISor® (vorozole), FEMARA® (letrozole; Novartis), and ARIMIDEX® (anastrozole; AstraZeneca); (iii) anti-androgens such as flutamide, nilutamide, bicalutamide, leuprolide, and goserelin; as well as troxacitabine (a 1,3-dioxolane nucleoside cytosine analog); (iv) protein kinase inhibitors such as ME inhibitors (WO 2007/044515); (v) lipid kinase inhibitors; (vi) antisense oligonucleotides, particularly those which inhibit expression of genes in signaling pathways implicated in aberrant cell proliferation, for example, PKC-alpha, Raf and H-Ras, such as oblimersen (GENASENSE®, Genta Inc.); (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAX1 D®; PROLEUKIN® rIL-2; topoisomerase 1 inhibitors such as LURTOTECAN®; ABARELIX® rmRH; (ix) anti-angiogenic agents such as bevacizumab (AVASTIN®, Genentech); and pharmaceutically acceptable salts, acids and derivatives of any of the above.

“Chemotherapeutic agents” may also include agents used in the treatment of leukemias, including alkylating agents such as chlorambucil, bendamustine hydrochloride or cyclophosphamide (CYTOXAN®)); purine analogs such as fludurabine (FLUDARA®), pentostatin (NIPENT®), cladribine or nelarabine; pyrimidine analogs such as cytarabine; corticosteroids such as prednisone, prednisolone or methylprednisolone, immunomodulatory agents such as lenalidomide or thalidomide, synthetic flavons such as flavopiridol, Bcl2 antagonists such as oblimersen or ABT-263, antibiotics such as doxorubicin (ADRIAMYCIN®), daunorubicin, idarubicin, or mitoxentrone; anti-metabolites such as methotrexate and clofarabine; tyrosine kinase inhibitors such as imatinib mesylate (GLEEVEC®), bosutinib, dasatinib, and nilotinib; a hypomethylating agents such as azacytidine or decitabine, an FLT3 inhibitor such as midostaurin, sorafenib, or AC220; arsenic trioxide; all-trans retinoic acid; vincristine sulfate; and monoclonal antibodies such as rituximab (RITUXAN®), ofatumumab, obinutuzumab, veltuzumab, ocrelizumab, lumiliximab or alemtuzumab (CAMPATH®); pharmaceutically acceptable salts, acids or derivatives of any of the above; as well as combinations of two or more of the above; as well as combinations of two or more of the above such as fludarabine plus cyclophosphamide (FC), cladribine plus cyclophosphamide (CC), fludarabine plus rituximab, fludarabine plus cyclophosphamide plus rituximab (FCR), and FCR plus alemtuzumab (CFAR). Chemotherapeutic agents may also include agents used in the treatment of multiple myeloma, including thalidomide, lenalidomide, bortezomib, dexamethesone, prednisone, and melphalan, as well as combinations of two or more of the above, such as thalidomide or lenalidomide plus dexamethasone, or bortezomib or lenalidomide plus melphalan and prednisone.

Also included in the definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide, and goserelin; as well as troxacitabine (a 1,3-dioxolane nucleoside cytosine analog); (iv) protein kinase inhibitors such as MEK inhibitors (WO 2007/044515); (v) lipid kinase inhibitors; (vi) antisense oligonucleotides, particularly those which inhibit expression of genes in signaling pathways implicated in aberrant cell proliferation, for example, PKC-alpha, Raf and H-Ras, such as oblimersen (GENASENSE®, Genta Inc.); (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN® rIL-2; topoisomerase 1 inhibitors such as LURTOTECAN®; ABARELIX® rmRH; (ix) anti-angiogenic agents such as bevacizumab (AVASTIN®, Genentech); and pharmaceutically acceptable salts, acids and derivatives of any of the above.

The term “prodrug” as used in this application refers to a precursor or derivative form of a compound of the invention that may be less cytotoxic to cells compared to the parent compound or drug and is capable of being enzymatically or hydrolytically activated or converted into the more active parent form. See, e.g., Wilman, “Prodrugs in Cancer Chemotherapy” Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). The prodrugs of this invention include, but are not limited to, phosphate-containing prodrugs, thiophosphate-containing prodrugs, sulfate-containing prodrugs, peptide-containing prodrugs, D-amino acid-modified prodrugs, glycosylated prodrugs, β-lactam-containing prodrugs, optionally substituted phenoxyacetamide-containing prodrugs, optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use in this invention include, but are not limited to, compounds of the invention and chemotherapeutic agents such as described above.

A “small molecule” is defined herein to have a molecular weight below about 500 Daltons.

An “isolated nucleic acid” is a nucleic acid, e.g., an RNA, DNA, or a mixed polymer, which is substantially separated from other genome DNA sequences as well as proteins or complexes such as ribosomes and polymerases, which naturally accompany a native sequence. An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a specific embodiment, a nucleic acid molecule(s) encoding an antibody provided herein is isolated or purified. The term embraces a nucleic acid sequence that has been removed from its naturally occurring environment, and includes recombinant or cloned DNA isolates and chemically synthesized analogues or analogues biologically synthesized by heterologous systems. A substantially pure molecule includes isolated forms of the molecule.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. “Oligonucleotide,” as used herein, generally refers to short, generally single-stranded, generally synthetic polynucleotides that are generally, but not necessarily, less than about 200 nucleotides in length. The terms “oligonucleotide” and “polynucleotide” are not mutually exclusive. The description above for polynucleotides is equally and fully applicable to oligonucleotides. The cell that produces an anti-C16orf54 antibody of the invention will include the parent hybridoma cell e.g., the hybridomas that are deposited with the ATCC, as well as bacterial and eukaryotic host cells into which nucleic acid encoding the antibodies have been introduced. Suitable host cells are disclosed below.

A “pre-cancerous cell” refers to a cell that has an abnormal appearance such as a difference in size or shape in comparison to cells of the surrounding tissue or normal cells of its cell type, but are not invasive. The appearance of pre-cancerous cells can be suggestive of an increased cancer risk. Pre-cancerous cells expressing C16orf54 can be identified using methods disclosed herein, which can include analyzing a sample of cells from a patient.

The term “package insert” is used to refer to instructions customarily included in commercial packages of therapeutic products, that contain information about the indications, usage, dosage, administration, contraindications and/or warnings concerning the use of such therapeutic products.

As used herein, the terms “prevent,” “preventing,” and “prevention” refer to the total or partial inhibition of the development, recurrence, onset or spread of a C16orf54-mediated disease and/or symptom related thereto, resulting from the administration of a therapy or combination of therapies provided herein (e.g., a combination of prophylactic or therapeutic agents, such as an antibody provided herein).

As used herein, the term “prophylactic agent” refers to any agent that can totally or partially inhibit the development, recurrence, onset or spread of a C16orf54-mediated disease and/or symptom related thereto in a subject. In certain embodiments, the term “prophylactic agent” refers to an anti-C16orf54 antibody provided herein. In certain other embodiments, the term “prophylactic agent” refers to an agent other than an anti-C16orf54 antibody provided herein. In certain embodiments, a prophylactic agent is an agent which is known to be useful to or has been or is currently being used to prevent a C16orf54-mediated disease and/or a symptom related thereto or impede the onset, development, progression and/or severity of a C16orf54-mediated disease and/or a symptom related thereto. In specific embodiments, the prophylactic agent is a humanized anti-C16orf54 antibody, such as a humanized anti-C16orf54 monoclonal antibody.

In certain embodiments, a “prophylactically effective serum titer” is the serum titer in a subject, preferably a human, that totally or partially inhibits the development, recurrence, onset or spread of a C16orf54-mediated disease and/or symptom related thereto in the subject.

In certain embodiments, a “therapeutically effective serum titer” is the serum titer in a subject, preferably a human, that reduces the severity, the duration and/or the symptoms associated with a C16orf54-mediated disease in the subject.

The term “recombinant antibody” refers to an antibody that is prepared, expressed, created or isolated by recombinant means. Recombinant antibodies can be antibodies expressed using a recombinant expression vector transfected into a host cell, antibodies isolated from a recombinant, combinatorial antibody library, antibodies isolated from an animal (e.g., a mouse or cow) that is transgenic and/or transchromosomal for human immunoglobulin genes (see e.g., Taylor, L. D. et al. (1992) Nucl. Acids Res. 20:6287-6295) or antibodies prepared, expressed, created or isolated by any other means that involves splicing of immunoglobulin gene sequences to other DNA sequences. Such recombinant antibodies can have variable and constant regions derived from human germline immunoglobulin sequences (See Kabat, E. A. et al. (1991) Sequences of Proteins of Immunological Interest, Fifth Edition, U.S. Department of Health and Human Services, NIH Publication No. 91-3242). In certain embodiments, however, such recombinant antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “serum titer” as used herein refers to an average serum titer in a population of least 10, such as at least 20, or at least 40 subjects, up to about 100, 1000 or more.

As used herein, the term “side effects” encompasses unwanted and adverse effects of a therapy (e.g., a prophylactic or therapeutic agent). Unwanted effects are not necessarily adverse. An adverse effect from a therapy (e.g., a prophylactic or therapeutic agent) might be harmful or uncomfortable or risky. Examples of side effects include, diarrhea, cough, gastroenteritis, wheezing, nausea, vomiting, anorexia, abdominal cramping, fever, pain, loss of body weight, dehydration, alopecia, dyspenea, insomnia, dizziness, mucositis, nerve and muscle effects, fatigue, dry mouth, and loss of appetite, rashes or swellings at the site of administration, flu-like symptoms such as fever, chills and fatigue, digestive tract problems and allergic reactions. Additional undesired effects experienced by patients are numerous and known in the art. Many are described in the Physician's Desk Reference (67^{th} ed., 2013).

As used herein, the terms “subject” and “patient” are used interchangeably. As used herein, in certain embodiments, a subject is a mammal, such as a non-primate (e.g., cows, pigs, horses, cats, dogs, rats, etc.) or a primate (e.g., monkey and human). In specific embodiments, the subject is a human. In one embodiment, the subject is a mammal (e.g., a human) having a C16orf54-mediated disease. In another embodiment, the subject is a mammal (e.g., a human) at risk of developing a C16orf54-mediated disease.

As used herein “substantially all” refers to refers to at least about 60%, at least about 70%, at least about 75%, at least about 80%, at least about 85%, at least about 90%, at least about 95%, at least about 98%, at least about 99%, or about 100%.

As used herein, the term “therapeutic agent” refers to any agent that can be used in treating, preventing or alleviating a disease, disorder or condition, including in the treatment, prevention or alleviation of one or more symptoms of a C16orf54-mediated disease, disorder, or condition and/or a symptom related thereto. In certain embodiments, a therapeutic agent refers to an antibody provided herein. In certain other embodiments, a therapeutic agent refers to an agent other than an antibody provided herein. In certain embodiments, a therapeutic agent is an agent which is known to be useful for, or has been or is currently being used for the treatment, prevention or alleviation of one or more symptoms of a C16orf54-mediated disease, disorder, condition, or a symptom related thereto.

The combination of therapies (e.g., use of therapeutic agents) can be more effective than the additive effects of any two or more single therapy. For example, a synergistic effect of a combination of therapeutic agents permits the use of lower dosages of one or more of the agents and/or less frequent administration of the agents to a subject with a C16orf54-mediated disease. The ability to utilize lower dosages of therapeutic therapies and/or to administer the therapies less frequently reduces the toxicity associated with the administration of the therapies to a subject without reducing the efficacy of the therapies in the prevention, treatment or alleviation of one or more symptom of a C16orf54-mediated disease. In addition, a synergistic effect can result in improved efficacy of therapies in the prevention, treatment or alleviation of one or more symptom of a C16orf54-mediated disease. Finally, synergistic effect of a combination of therapies (e.g., therapeutic agents) may avoid or reduce adverse or unwanted side effects associated with the use of any single therapy.

As used herein, the term “therapy” refers to any protocol, method and/or agent that can be used in the prevention, management, treatment and/or amelioration of a C16orf54-mediated disease. In certain embodiments, the terms “therapies” and “therapy” refer to a biological therapy, supportive therapy, and/or other therapies useful in the prevention, management, treatment and/or amelioration of a C16orf54-mediated disease known to one of skill in the art such as medical personnel.

The term “thiol,” as used herein, refers to the radical —SH.

The term “alkyl,” as used herein, means a straight, branched chain, or cyclic (in this case, it would also be known as “cycloalkyl”) hydrocarbon containing from 1-10 carbon atoms. Examples of alkyl include, but are not limited to, methyl, ethyl, n-propyl, iso-propyl, n-butyl, sec-butyl, tert-butyl, n-pentyl, isopentyl, neopentyl, n-hexyl, 3-methylhexyl, 2,2-dimethylpentyl, 2,3-dimethylhexyl, n-heptyl, n-octyl, n-nonyl, and n-decyl. In certain embodiments, alkyl groups are optionally substituted.

The term “C₁₋₆alkyl,” as used herein, means a straight, branched chain, or cyclic (in this case, it would also be known as “cycloalkyl”) hydrocarbon containing from 1-6 carbon atoms.

The term “C₁₋₃alkyl,” as used herein, means a straight or branched chain hydrocarbon containing from 1-3 carbon atoms.

The term “alkenyl,” as used herein, means a straight, branched chain, or cyclic (in which case, it would also be known as a “cycloalkenyl”) hydrocarbon containing from 2-10 carbons and containing at least one carbon-carbon double bond formed by the removal of two hydrogens. In some embodiments, depending on the structure, an alkenyl group is a monoradical or a diradical (e.g., an alkenylene group). In some embodiments, alkenyl groups are optionally substituted. Examples of alkenyl include, but are not limited to, ethenyl, 2-propenyl, 2-methyl-2-propenyl, 3-butenyl, 4-pentenyl, 5-hexenyl, 2-heptenyl, and 2-methyl-1-heptenyl. In certain embodiments, alkenyl groups are optionally substituted.

The term “C₂₋₆ alkenyl,” as used herein, means a straight, branched chain, or cyclic (in this case, it would also be known as “cycloalkyl”) hydrocarbon containing from 2-6 carbon atoms and at least one carbon-carbon double bond formed by the removal of two hydrogens.

The term “alkoxy,” as used herein, means an alkyl group, as defined herein, appended to the parent molecular moiety through an oxygen atom. Examples of alkoxy include, but are not limited to, methoxy, ethoxy, propoxy, 2-propoxy, butoxy, tert-butoxy, pentyloxy, and hexyloxy.

An “amino acid” (or AA) or amino acid residue include but are not limited to the 20 naturally occurring amino acids acids commonly designated by three letter symbols and also includes 4 hydroxyproline, hydroxyysine, demosine, isodemosine, 3-methylhistidine, norvalin, beta-alanine, gamma-aminobutyric acid, homocysteine, homoserine, ornithine and methionine sulfone. The amino acid residue of the present application also include the corresponding N-methyl amino acids, such as —N(CH₃)CH₂C(O)O—, —NHC(O)CH₂CH₂CH(NHCH₃)C(O)O—, etc. The amino acids, dipeptides, tripeptides, oligomers and polypeptides designated as -(AA)ᵣ- of the present application may include the corresponding non-N-alkylated amino acids and peptides (such as non-N-methylated amino acids in the peptides), as well as a mixture of the non-N-alkylated amino acids and the N-alkylated amino acids of the peptides.

The term “chemical group,” as used herein, refers to two or more atoms bound together as a single unit and forming part of a molecule.

The term “cycloalkyl,” as used herein, means a monocyclic or polycyclic radical that contains only carbon and hydrogen, and includes those that are saturated, partially unsaturated, or fully unsaturated. Cycloalkyl groups include groups having from 3 to 10 ring atoms.

The term “detectable probe,” as used herein, refers to a composition that provides a detectable signal. The term includes, without limitation, any fluorophore, chromophore, radiolabel, enzyme, antibody or antibody fragment, and the like, that provide a detectable signal via its activity.

The term “diagnostic agent” refers to a substance administered to a subject that aids in the diagnosis of a disease. Such substances can be used to reveal, pinpoint, and/or define the localization of a disease causing process. In certain embodiments, a diagnostic agent includes a substance that is conjugated to an antibody provided herein, that when administered to a subject or contacted to a sample from a subject aids in the diagnosis of cancer, tumor formation, or any other C16orf54-mediated disease.

The term “detectable agent” refers to a substance that can be used to ascertain the existence or presence of a desired molecule, such as an antibody provided herein, in a sample or subject. A detectable agent can be a substance that is capable of being visualized or a substance that is otherwise able to be determined and/or measured (e.g., by quantitation).

The term “electrophilic leaving group,” as used herein, refers to a leaving group that accepts an electron pair to make a covalent bond. In general, electrophiles are susceptible to attack by complementary nucleophiles, including the reduced thiols from the disulfide bond of an antibody.

The term “electrophilic leaving group that reacts selectively with thiols,” as used herein, refers to electrophilic leaving group that reacts selectively with thiols, over other nucleophiles. In certain embodiments, an electrophilic leaving group that reacts selectively with thiols reacts selectively with the reduced thiols from the disulfide bond of an antibody.

The term “encode” or grammatical equivalents thereof as it is used in reference to nucleic acid molecule refers to a nucleic acid molecule in its native state or when manipulated by methods well known to those skilled in the art that can be transcribed to produce mRNA, which is then translated into a polypeptide and/or a fragment thereof. The antisense strand is the complement of such a nucleic acid molecule, and the encoding sequence can be deduced therefrom.

The term “excipient” as used herein refers to an inert substance which is commonly used as a diluent, vehicle, preservative, binder, or stabilizing agent, and includes, but not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). See, also, Remington's Pharmaceutical Sciences (1990) Mack Publishing Co., Easton, Pa., which is hereby incorporated by reference in its entirety.

In the context of a peptide or polypeptide, the term “fragment” as used herein refers to a peptide or polypeptide that comprises less than the full length amino acid sequence. Such a fragment may arise, for example, from a truncation at the amino terminus, a truncation at the carboxy terminus, and/or an internal deletion of a residue(s) from the amino acid sequence. Fragments may, for example, result from alternative RNA splicing or from in vivo protease activity. In certain embodiments, C16orf54 fragments include polypeptides comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least contiguous 100 amino acid residues, at least 125 contiguous amino acid residues, at least 150 contiguous amino acid residues, at least 175 contiguous amino acid residues, at least 200 contiguous amino acid residues, or at least 250 contiguous amino acid residues of the amino acid sequence of a C16orf54 polypeptide or an antibody that binds to a C16orf54 polypeptide. In a specific embodiment, a fragment of a C16orf54 polypeptide or an antibody that binds to a C16orf54 antigen retains at least 1, at least 2, or at least 3 functions of the polypeptide or antibody.

The term “leaving group,” as used herein, refers to any group that leaves in the course of a chemical reaction involving the group as described herein and includes but is not limited to halogen, sulfonates (brosylate, mesylate, tosylate triflate etc . . . ), p-nitrobenzoate and phosphonate groups, for example.

The term “light chain” when used in reference to an antibody refers to a polypeptide chain of about 25 kDa, wherein the amino-terminal portion includes a variable region of about 100 to about 110 or more amino acids and a carboxy-terminal portion that includes a constant region. The approximate length of a light chain is 211 to 217 amino acids. There are two distinct types, referred to as kappa (K) of lambda (A) based on the amino acid sequence of the constant domains. Light chain amino acid sequences are well known in the art. A light chain can be a human light chain.

A “linker” (noted as L or L¹, L² and L³) is a molecule with two reactive termini, one for conjugation to an antibody or to another linker and the other for conjugation to a cytotoxic agent. The antibody conjugation reactive terminus of the linker is typically a site that is capable of conjugation to the antibody through a cysteine thiol or lysine amine group on the antibody, and so is typically a thiol-reactive group such as a double bond (as in maleimide) or a leaving group such as a chloro, bromo or iodo or an R-sulfanyl group or sulfonyl group, or an amine-reactive group such as a carboxyl group or as defined herein; while the antibody conjugation reactive terminus of the linker is typically a site that is capable of conjugation to the cytotoxic agent through formation of an amide bond with a basic amine or carboxyl group on the cytotoxin, and so is typically a carboxyl or basic amine group. In one embodiment, when the term “linker” is used in describing the linker in conjugated form, one or both of the reactive termini will be absent (such as the leaving group of the thiol-reactive group) or incomplete (such as the being only the carbonyl of the carboxylic acid) because of the formation of the bonds between the linker and/or the cytotoxic agent.

As used herein, the terms “manage,” “managing,” and “management” refer to the beneficial effects that a subject derives from a therapy (e.g., a prophylactic or therapeutic agent), which does not result in a cure of the disease. In certain embodiments, a subject is administered one or more therapies (e.g., prophylactic or therapeutic agents, such as an antibody provided herein) to “manage” a C16orf54-mediated disease, one or more symptoms thereof, so as to prevent the progression or worsening of the disease.

The term “thiol,” as used herein, refers to the radical —SH.

“Tubulysin” includes both the natural products described as tubulysins, such as by Sasse et al. and other authors mentioned in the Description of the related art, and also the tubulysin analogs described in US Patent Application Publication No. US 2011/0021568 A1. Tubulysins disclosed in the present application are noted herein and may include the tubulysins of the formulae T3 and T4, and other tubulysins where the terminal N-methylpiperidine has been replaced by an unsubstituted piperidine, allowing amide bond formation with a linker.

The term “about” or “approximately” means within 20%, such as within 10%, or within 5% (or 1% or less) of a given value or range.

As used herein, “administer” or “administration” refers to the act of injecting or otherwise physically delivering a substance as it exists outside the body (e.g., an anti-C16orf54 antibody provided herein) into a patient, such as by mucosal, intradermal, intravenous, intramuscular delivery and/or any other method of physical delivery described herein or known in the art. When a disease, or a symptom thereof, is being treated, administration of the substance typically occurs after the onset of the disease or symptoms thereof. When a disease, or symptoms thereof, are being prevented, administration of the substance typically occurs before the onset of the disease or symptoms thereof.

In the context of a polypeptide, the term “analog” as used herein refers to a polypeptide that possesses a similar or identical function as a C16orf54 polypeptide, a fragment of a C16orf54 polypeptide, or an anti-C16orf54 antibody but does not necessarily comprise a similar or identical amino acid sequence of a C16orf54 polypeptide, a fragment of a C16orf54 polypeptide, or an anti-C16orf54 antibody, or possess a similar or identical structure of a C16orf54 polypeptide, a fragment of a C16orf54 polypeptide, or an anti-C16orf54 antibody. A polypeptide that has a similar amino acid sequence refers to a polypeptide that satisfies at least one of the following: (a) a polypeptide having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of a C16orf54 polypeptide (e.g., SEQ ID NO:1079), a fragment of a C16orf54 polypeptide, or an anti-C16orf54 antibody described herein; (b) a polypeptide encoded by a nucleotide sequence that hybridizes under stringent conditions to a nucleotide sequence encoding a C16orf54 polypeptide, a fragment of a C16orf54 polypeptide, or an anti-C16orf54 antibody (or VH or VL region thereof) described herein of at least 5 amino acid residues, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues (see, e.g., Sambrook et al. (2001) Molecular Cloning: A Laboratory Manual, Cold Spring Harbor Laboratory Press, Cold Spring Harbor, N.Y.; Maniatis et al. (1982) Molecular Cloning: A Laboratory Manual, Cold Spring Harbor Press, Cold Spring Harbor, N.Y.); and (c) a polypeptide encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% identical to the nucleotide sequence encoding a C16orf54 polypeptide, a fragment of a C16orf54 polypeptide, or an anti-C16orf54 antibody (or VH or VL region thereof) described herein. A polypeptide with similar structure to a C16orf54 polypeptide, a fragment of a C16orf54 polypeptide, or an anti-C16orf54 antibody described herein refers to a polypeptide that has a similar secondary, tertiary or quaternary structure of a C16orf54 polypeptide, a fragment of a C16orf54, or a C16orf54 antibody described herein. The structure of a polypeptide can determined by methods known to those skilled in the art, including but not limited to, X-ray crystallography, nuclear magnetic resonance, and crystallographic electron microscopy.

As used herein, the term “composition” is intended to encompass a product containing the specified ingredients (e.g., an antibody provided herein) in, optionally, the specified amounts, as well as any product which results, directly or indirectly, from combination of the specified ingredients in, optionally, the specified amounts.

In the context of a polypeptide, the term “derivative” as used herein refers to a polypeptide that comprises an amino acid sequence of a C16orf54 polypeptide, a fragment of a C16orf54 polypeptide, or an antibody that binds to a C16orf54 polypeptide which has been altered by the introduction of amino acid residue substitutions, deletions or additions. The term “derivative” as used herein also refers to a C16orf54 polypeptide, a fragment of a C16orf54 polypeptide, or an antibody that binds to a C16orf54 polypeptide which has been chemically modified, e.g., by the covalent attachment of any type of molecule to the polypeptide. For example, but not by way of limitation, a C16orf54 polypeptide, a fragment of a C16orf54 polypeptide, or a C16orf54 antibody may be chemically modified, e.g., by glycosylation, acetylation, pegylation, phosphorylation, amidation, derivatization by known protecting/blocking groups, proteolytic cleavage, linkage to a cellular ligand or other protein, etc. The derivatives are modified in a manner that is different from naturally occurring or starting peptide or polypeptides, either in the type or location of the molecules attached. Derivatives further include deletion of one or more chemical groups which are naturally present on the peptide or polypeptide. A derivative of a C16orf54 polypeptide, a fragment of a C16orf54 polypeptide, or a C16orf54 antibody may be chemically modified by chemical modifications using techniques known to those of skill in the art, including, but not limited to specific chemical cleavage, acetylation, formulation, metabolic synthesis of tunicamycin, etc. Further, a derivative of a C16orf54 polypeptide, a fragment of a C16orf54 polypeptide, or a C16orf54 antibody may contain one or more non-classical amino acids. A polypeptide derivative possesses a similar or identical function as a C16orf54 polypeptide, a fragment of a C16orf54 polypeptide, or a C16orf54 antibody described herein.

### Compositions and Methods of Making the Same

Antibodies that bind to C16orf54 are provided. Immunoconjugates comprising anti-C16orf54 antibodies are also provided. Antibodies and immunoconjugates of the invention are useful, e.g., for the diagnosis or treatment of disorders associated with altered expression, e.g., increased expression, of C16orf54. In certain embodiments, antibodies or immunoconjugates of the invention are useful for the diagnosis or treatment of a cell proliferative disorder, such as cancer.

Provided herein are antibodies that bind to a C16orf54 polypeptide, a C16orf54 polypeptide fragment, C16orf54 peptide, or a C16orf54 epitope. In some embodiments, the anti-C16orf54 antibodies bind to the extracellular domain (ECD) of C16orf54. Also provided are antibodies that competitively block an anti-C16orf54 antibody provided herein from binding to a C16orf54 polypeptide. The anti-C16orf54 antibodies provided herein can also be conjugated or recombinantly fused to a diagnostic agent, detectable agent or therapeutic agent (e.g., antibody-drug conjugate). Further provided are compositions comprising an anti-C16orf54 antibody. For example, a detectable agent may be a detectable probe.

Also provided herein are isolated nucleic acid molecules encoding a VH chain, VL chain, VH domain, VL domain, VH CDR1, VH CDR2, VH CDR3, VL CDR1, VL CDR2, and/or VL CDR3 of anti-C16orf54 antibodies that bind to a C16orf54 polypeptide, a C16orf54 polypeptide fragment, a C16orf54 peptide or a C16orf54 epitope. Further provided are vectors and host cells comprising nucleic acid molecules encoding anti-C16orf54 antibodies that bind to a C16orf54 polypeptide, a C16orf54 polypeptide fragment, a C16orf54 peptide or a C16orf54 epitope. Also provided are methods of making antibodies that bind to a C16orf54 polypeptide, a C16orf54 polypeptide fragment, a C16orf54 peptide or a C16orf54 epitope.

Methods of using the anti-C16orf54 antibodies are provided. The methods include treating, preventing or alleviating a disease, disorder or condition, including treating, preventing ro alleviating one or more symptoms of a disease, disorder or condition in a subject or inhibiting the growth of a cell having cell surface expression of a C16orf54 polypeptide. Additional methods provided include using an anti-C16orf54 antibody provided herein, for example, as an unconjugated antibody or conjugated antibody (ADC), with anti-tumor activity to mediate anti-tumor effects. In certain embodiments, the anti-C16orf54 antibodies provided herein directly kill C16orf54-bearing tumor cells (e.g., via antibody-dependent cellular cytotoxicity (ADCC) and/or complement-dependent cytotoxicity (CDC). In certain embodiments, antibody drug conjugates (ADCs) comprising anti-C16orf54 antibodies provided herein directly kill C16orf54-bearing tumor cells (e.g., by binding to tumor cells expressing C16orf54 and allowing internalization of the cytotoxic drug). Additional methods provided include using an anti-C16orf54 antibody to modulate a C16orf54 mediated disease or disorder detecting C16orf54 in a sample.

Anti-C16orf54 Antibodies

In one embodiment, the present invention provides anti-C16orf54 antibodies that may find use herein as therapeutic agents. Exemplary antibodies include polyclonal, monoclonal, humanized, human, bispecific, and heteroconjugate antibodies, as well as variants thereof having improved affinity or other properties.

In some embodiments, provided herein are antibodies that bind to C16orf54, including a C16orf54 polypeptide, a C16orf54 polypeptide fragment, a C16orf54 peptide or a C16orf54 epitope. In some embodiments the anti-C16orf54 antibodies are humanized antibodies (e.g., comprising human constant regions) that bind C16orf54, including C16orf54 polypeptide, a C16orf54 polypeptide fragment, a C16orf54 peptide or a C16orf54 epitope.

In certain embodiments, the anti-C16orf54 antibody comprises a VH region, VL region, VH CDR1, VH CDR2, VH CDR3, VL CDR1, VL CDR2, and/or VL CDR3 of any one of the murine monoclonal antibodies described herein, such as an amino acid sequence depicted in Tables 1-29. Accordingly, in some embodiments, the isolated antibody or functional fragment thereof provided herein comprises one, two, or three heavy chain CDRs and/or one, two, or three light chain CDRs from: (a) the antibody designated R29-7-2A; (b) the antibody designated R29-7-1C; (c) the antibody designated R29-67-7A; (d) the antibody designated R29-8-136C; (e) the antibody designated R29-8-57B; (f) the antibody designated R29-7-54C; (g) the antibody designated R29-7-53A; (h) the antibody designated R29-8-50C; (i) the antibody designated R29-8-19B; (j) the antibody designated R29-8-58C; (k) the antibody designated R29-8-9B; (l) the antibody designated R29-8-28C; (m) the antibody designated R29-8-120B; (n) the antibody designated R29-8-75B; (o) the antibody designated R29-8-36C; (p) the antibody designated R29-8-12A; (q) the antibody designated R29-8-93B; (r) the antibody designated R29-8-51B; (s) the antibody designated R29-8-30A; (t) the antibody designated R29-8-18B; (u) the antibody designated R29-7-38C; (v) the antibody designated R29-7-49A; (w) the antibody designated R29-7-13A; or (x) the antibody designated R29-67-4A.

TABLE 1
VH/VL Sequences for Group I Antibodies

| | | VH CDR Sequences | | | VL CDR Sequences | | |
|---|---|---|---|---|---|---|---|
| # | Antibody | VH CDR1 | VH CDR2 | VH CDR3 | VL CDR1 | VL CDR2 | VL CDR3 |
| | | Consensus Sequences | | | Consensus Sequences | | |
| | | GFTGSX₁YA | ITGX₁GGX₂X₃ | X₁RGWDENDX₂ | QSLX₁X₂SNGNTY | KVS | SQX₁THVPWT |
| | | (SEQ ID NO: 67) | (SEQ ID NO: 68) | (SEQ ID NO: 69) | (SEQ ID NO: 70) | (SEQ ID NO: | (SEQ ID |
| | | | | | | 71) | NO: 72) |
| | | Individual Sequences | | | Individual Sequences | | |
| 1 | R29-8-9B | GFTFSSYA | ITGGGGT | ARGWDENDY | QSLVFSNGNTY | KVS | SQSTHVPWT |
| | | (SEQ ID NO: 76) | (SEQ ID NO: 97) | (SEQ ID NO: 103) | (SEQ ID NO: 106) | (SEQ ID NO: | (SEQ ID |
| | | | | | | 71) | NO: 109) |
| 2 | R29-8-93B | GFTFSSYA | ITGGGGT | ARGWDENDY | — | — | — |
| | | (SEQ ID NO: 76) | (SEQ ID NO: 97) | (SEQ ID NO: 103) | | | |
| 3 | R29-8-51B | GFTFSSYA | ITGGGGT | ARGWDENDY | — | — | — |
| | | (SEQ ID NO: 76) | (SEQ ID NO: 97) | (SEQ ID NO: 103) | | | |
| 4 | R29-8-30A | GFTFSNYA | ITGGGST | ARGWDENDY | — | — | — |
| | | (SEQ ID NO: 89) | (SEQ ID NO: 98) | (SEQ ID NO: 103) | | | |
| 5 | R29-8-120B | GFTFSIYA | ITGGGTN | ARGWDENDY | QSLVYSNGNTY | KVS | SQSTHVPWT |
| | | (SEQ ID NO: 95) | (SEQ ID NO: 99) | (SEQ ID NO: 103) | (SEQ ID NO: 107) | (SEQ ID NO: | (SEQ ID |
| | | | | | | 71) | NO: 109) |
| 6 | R29-8-18B | GFTFSTYA | ITSGGST | ARGWDENDY | — | — | — |
| | | (SEQ ID NO: 96) | (SEQ ID NO: 100) | (SEQ ID NO: 103) | | | |
| 7 | R29-8-28C | GFTFSNYA | ITSGGRT | GRGWDENDY | QSLVYSNGNTY | KVS | SQTTHVPWT |
| | | (SEQ ID NO: 89) | (SEQ ID NO: 101) | (SEQ ID NO: 104) | (SEQ ID NO: 107) | (SEQ ID NO: | (SEQ ID |
| | | | | | | 71) | NO: 110) |
| 8 | R29-8-19B | GFTFSSYA | ITSGGRT | TRGWDENDL | QSLVYSNGNTY | KVS | SQTTHVPWT |
| | | (SEQ ID NO: 76) | (SEQ ID NO: 101) | (SEQ ID NO: 105) | (SEQ ID NO: 107) | (SEQ ID NO: | (SEQ ID |
| | | | | | | 71) | NO: 110) |
| 9 | R29-8-50C | GFTFSSYA | ITSGGRT | TRGWDENDL | QSLVYSNGNTY | KVS | SQTTHVPWT |
| | | (SEQ ID NO: 76) | (SEQ ID NO: 101) | (SEQ ID NO: 105) | (SEQ ID NO: 107) | (SEQ ID NO: | (SEQ ID |
| | | | | | | 71) | NO: 110) |
| 10 | R29-8-12A | GFTFSSYA | ITSGGRT | TRGWDENDL | QSLVYSNGNTY | KVS | SQTTHVPWT |
| | | (SEQ ID NO: 76) | (SEQ ID NO: 101) | (SEQ ID NO: 105) | (SEQ ID NO: 107) | (SEQ ID NO: | (SEQ ID |
| | | | | | | 71) | NO: 110) |
| 11 | R29-8-36C | GFTFSSYA | ITSGGRS | TRGWDENDL | QSLLYSNGNTY | KVS | SQTTHVPWT |
| | | (SEQ ID NO: 76) | (SEQ ID NO: 102) | (SEQ ID NO: 105) | (SEQ ID NO: 108) | (SEQ ID NO: | (SEQ ID |
| | | | | | | 71) | NO: 110) |
| 12 | R29-8-58C | GFTFSSYA | ITSGGRS | TRGWDENDL | QSLLYSNGNTY | KVS | SQTTHVPWT |
| | | (SEQ ID NO: 76) | (SEQ ID NO: 102) | (SEQ ID NO: 105) | (SEQ ID NO: 108) | (SEQ ID NO: | (SEQ ID |
| | | | | | | 71) | NO: 110) |

Residue designated “X” represents any naturally occurring amino acid.

TABLE 2
VH/VL Sequences for Group II Antibodies

| | | VH CDR Sequences | | | VL CDR Sequences | | |
|---|---|---|---|---|---|---|---|
| | Antibody | VH CDR1 | VH CDR2 | VH CDR3 | VL CDR1 | VL CDR2 | VL CDR3 |
| # | | Individual Sequences | | | Individual Sequences | | |
| 13 | R29-8-75B | GFTFSRFG | ISSGSSTI | ARVDYDVALAY | QSIVHRNGNTY | KVS | FQGSQWT |
| | | (SEQ ID | (SEQ ID NO: 74) | (SEQ ID NO: 75) | (SEQ ID NO: 76) | (SEQ ID NO: 71) | (SEQ ID NO: 78) |
| | | NO: 73) | | | | | |

TABLE 3
VH/VL Sequences for Group III Antibodies

| | | VH CDR Sequences | | | VL CDR Sequences | | |
|---|---|---|---|---|---|---|---|
| # | Antibody | VH CDR1 | VH CDR2 | VH CDR3 | VL CDR1 | VL CDR2 | VL CDR3 |
| | | Consensus Sequences | | | Consensus Sequences | | |
| | | GYSITSX₁YA | IX₁YSGX₂X₃ | AREX₁YDX₂X₃X₄YX₅ | QX₁LLYSX₂NQKNY | WAS | QQYYX₁YRT |
| | | (SEQ ID | (SEQ ID | MDY | (SEQ ID NO: 82) | (SEQ ID NO: 83) | (SEQ ID NO: 84) |
| | | NO: 79) | NO: 80) | (SEQ ID NO: 81) | | | |
| | | Individual Sequences | | | Individual Sequences | | |
| 14 | R29-8-57B | GYSITSDYA | INYSGST | ARERYDGVYYGMDY | QSLLYSSNQKNY | WAS | QQYYSYRT |
| | | (SEQ ID | (SEQ ID | (SEQ ID NO: 118) | (SEQ ID NO: 94) | (SEQ ID NO: 83) | (SEQ ID NO: 124) |
| | | NO: 111) | NO: 113) | | | | |
| 15 | R29-8-136C | GYSITSDYA | INYSGSS | ARERYDGVYYGMDY | QSLLYSSNQKNY | WAS | QQYYSYRT |
| | | (SEQ ID | (SEQ ID | (SEQ ID NO: 118) | (SEQ ID NO: 94) | (SEQ ID NO: 83) | (SEQ ID NO: 124) |
| | | NO: 111) | NO: 114) | | | | |
| 16 | R29-7-1C | GYSITSDYA | ISYSGSI | AREKYDNYYYAMDY | QNLLYSTNQKNY | WAS | QQYYSYRT |
| | | (SEQ ID | (SEQ ID | (SEQ ID NO: 119) | (SEQ ID NO: 122) | (SEQ ID NO: 83) | (SEQ ID NO: 124) |
| | | NO: 111) | NO: 115) | | | | |
| 17 | R29-67-7A | GYSITSDYA | INYSGRT | ARENYDYEFYAMDY | QNLLYSSNQKNY | WAS | QQYYIYRT |
| | | (SEQ ID | (SEQ ID | (SEQ ID NO: 120) | (SEQ ID NO: 123) | (SEQ ID NO: 83) | (SEQ ID NO: 125) |
| | | NO: 111) | NO: 116) | | | | |
| 18 | R29-67-4A | GYSITSVYA | ISYSGIT | ATTGTR* | — | — | — |
| | | (SEQ ID | (SEQ ID | (SEQ ID NO: 121) | | | |
| | | NO: 112) | NO: 117) | | | | |

Residue designated “X” represents any naturally occurring amino acid.
*Not included in consensus sequence.

TABLE 4
VH/VL Sequences for Group IV Antibodies

| | | VH CDR Sequences | | | VL CDR Sequences | | |
|---|---|---|---|---|---|---|---|
| # | Antibody | VH CDR1 | VH CDR2 | VH CDR3 | VL CDR1 | VL CDR2 | VL CDR3 |
| | | Consensus Sequences | | | Consensus Sequences | | |
| | | GFSLTDYX₁ | IWGGGX₁T | AKHEEVSRFAX₁ | QSLVYSNGNSY | KVS | SQSTHIPLT |
| | | (SEQ ID | (SEQ ID NO: 86) | (SEQ ID NO: 87) | (SEQ ID | (SEQ ID NO: 71) | (SEQ ID NO: 90) |
| | | NO: 85) | | | NO: 88) | | |
| | | Individual Sequences | | | Individual Sequences | | |
| 19 | R29-7A-53A | GFSLTDYA | IWGGGRT | AKHEEVSRFAY | QSLVYSNGNSY | KVS | SQSTHIPLT |
| | | (SEQ ID | (SEQ ID NO: 128) | (SEQ ID NO: 130) | (SEQ ID | (SEQ ID NO: 71) | (SEQ ID NO: 90) |
| | | NO: 126) | | | NO: 88) | | |
| 20 | R29-7A-54C | GFSLTDYA | IWGGGRT | AKHEEVSRFAY | QSLVYSNGNSY | KVS | SQSTHIPLT |
| | | (SEQ ID | (SEQ ID NO: 128) | (SEQ ID NO: 130) | (SEQ ID | (SEQ ID NO: 71) | (SEQ ID NO: 90) |
| | | NO: 126) | | | NO: 88) | | |
| 21 | R29-7A-38C | GFSLTDYA | IWGGGRT | AKHEEVSRFAH | — | — | — |
| | | (SEQ ID | (SEQ ID NO: 128) | (SEQ ID NO: 77) | | | |
| | | NO: 126) | | | | | |
| 22 | R29-7A-49A | GFSLTDYG | IWGGGGT | AKHEEVSRFAY | — | — | — |
| | | (SEQ ID | (SEQ ID NO: 129) | (SEQ ID NO: 130) | | | |
| | | NO: 127) | | | | | |
| 23 | R29-7A-13A | GFSLTDYA | IWGGGRT | AKHEEVSRFAY | — | — | — |
| | | (SEQ ID | (SEQ ID NO: 128) | (SEQ ID NO: 130) | | | |
| | | NO: 126) | | | | | |

Residue designated “X” represents any naturally occurring amino acid.

TABLE 5
VH/VL Sequences for Group V Antibodies

| | | VH CDR Sequences | | | VL CDR Sequences | | |
|---|---|---|---|---|---|---|---|
| | Antibody | VH CDR1 | VH CDR2 | VH CDR3 | VL CDR1 | VL CDR2 | VL CDR3 |
| # | | Individual Sequences | | | Individual Sequences | | |
| 24 | R29-7-2A | GFSFNTHA | IRSKSNNYAR | VKQGDGGFAY | QSLLYSSNQKNY | WAS | QQYYSYPPT |
| | | (SEQ ID | (SEQ ID | (SEQ ID | (SEQ ID NO: 94) | (SEQ ID NO: 83) | (SEQ ID NO: 96) |
| | | NO: 91) | NO: 92) | NO: 93) | | | |

TABLE 6
Antibody R29-8-9B CDR Sequences

| | | IMGT | Kabat | Chothia | Contact | AbM |
|---|---|---|---|---|---|---|
| VH CDR | VH CDR1 | GFTFSSYA | SYAMS | GFTFSSY | SSYAMS | GFTFSSYAMS |
| Seq. | | (SEQ ID NO: 76) | (SEQ ID | (SEQ ID | (SEQ ID NO: 166) | (SEQ ID |
| | | | NO: 147) | NO: 161) | | NO: 172) |
| | VH CDR2 | ITGGGGT | SITGGGGTYYP | GGG | WVASITGGGGTY | SITGGGGTY |
| | | (SEQ ID NO: 97) | DSVKG | (SEQ ID | (SEQ ID NO: 167) | (SEQ ID |
| | | | (SEQ ID | NO: 162) | | NO: 173) |
| | | | NO: 148) | | | |
| | VH CDR3 | ARGWDENDY | GWDENDY | WDEND | ARGWDEND | GWDENDY |
| | | (SEQ ID NO: 103) | (SEQ ID | (SEQ ID | (SEQ ID NO: 168) | (SEQ ID |
| | | | NO: 149) | NO: 163) | | NO: 149) |
| VL CDR | VL CDR1 | QSLVFSNGNTY | RSSQSLVFSN | SQSLVFSNGNTY | VFSNGNTYLHWY | RSSQSLVFS |
| Seq. | | (SEQ ID NO: 106) | GNTYLH | (SEQ ID | (SEQ ID NO: 169) | NGNTYLH |
| | | | (SEQ ID | NO: 164) | | (SEQ ID |
| | | | NO: 150) | | | NO: 150) |
| | VL CDR2 | KVS | KVSNRFS | KVS | LLIYKVSNRF | KVSNRFS |
| | | (SEQ ID NO: 71) | (SEQ ID | (SEQ ID NO: 71) | (SEQ ID NO: 170) | (SEQ ID |
| | | | NO: 160) | | | NO: 160) |
| | VL CDR3 | SQSTHVPWT | SQSTHVPWT | STHVPW | SQSTHVPW | SQSTHVPWT |
| | | (SEQ ID NO: 109) | (SEQ ID | (SEQ ID | (SEQ ID NO: 171) | (SEQ ID |
| | | | NO: 109) | NO: 165) | | NO: 109) |

VH Sequence:
MNYGFSLIFLVLVLKGVQCEVKLVESGGGVVKPGGSLKLSCAASGFTFSSYAMSWFRQTSEMSLEWVASITGGGGTYYPDSVKGRFTISRDTVRNILYLQMRSLRSGDTAIYYCARGWDENDYWGQGTTLTVSS (SEQ ID NO: 44)
VL Sequence:
MKLPVRLLVLMFWIPASRSDVVMTQIPLSLPVSLGDQASISCRSSQSLVFSNGNTYLHWYFQKPGQSPKLLIYKVSNRFSGVPDRFSGSGSGTDFILRISRVEAEDLGVYFCSQSTHVPWTFGGGTKLEIK (SEQ ID NO: 46)

TABLE 7
Antibody R29-8-93B CDR Sequences

| | | IMGT | Kabat | Chothia | Contact | AbM |
|---|---|---|---|---|---|---|
| VH CDR | VH CDR1 | GFTFSSYA | SYAMS | GFTFSSY | SSYAMS | GFTFSSYAMS |
| Seq. | | (SEQ ID NO: 76) | (SEQ ID | (SEQ ID | (SEQ ID NO: 166) | (SEQ ID |
| | | | NO: 147) | NO: 161) | | NO: 172) |
| | VH CDR2 | ITGGGGT | SITGGGGTY | GGG | WVASITGGGGTY | SITGGGGTY |
| | | (SEQ ID NO: 97) | YPDSVKG | (SEQ ID | (SEQ ID NO: 167) | (SEQ ID |
| | | | (SEQ ID | NO: 162) | | NO: 173) |
| | | | NO: 148) | | | |
| | VH CDR3 | ARGWDENDY | GWDENDY | WDEND | ARGWDEND | GWDENDY |
| | | (SEQ ID NO: 103) | (SEQ ID | (SEQ ID | (SEQ ID NO: 168) | (SEQ ID |
| | | | NO: 149) | NO: 163) | | NO: 149) |

VH Sequence:
EVKLVESGGGVVKPGGSLKLSCTASGFTFSSYAMSWFRQTSEMRLEWVASITGGGGTYYPDSVKGRFTISRDTVRNILYLQMRSLRSGDTAIYYCARGWDENDYWGQGTPLTVSS (SEQ ID NO: 132)

TABLE 8
Antibody R29-8-51B CDR Sequences

| | | IMGT | Kabat | Chothia | Contact | AbM |
|---|---|---|---|---|---|---|
| VH CDR | VH CDR1 | GFTFSSYA | SYAMS | GFTFSSY | SSYAMS | GFTFSSYAMS |
| Seq. | | (SEQ ID | (SEQ ID | (SEQ ID | (SEQ ID NO: 166) | (SEQ ID |
| | | NO: 76) | NO: 147) | NO: 161) | | NO: 172) |
| | VH CDR2 | ITGGGGT | SITGGGGTYY | GGG | WVASITGGGGTY | SITGGGGTY |
| | | (SEQ ID | PDSVKG | (SEQ ID | (SEQ ID NO: 167) | (SEQ ID |
| | | NO: 97) | (SEQ ID | NO: 162) | | NO: 173) |
| | | | NO: 148) | | | |
| | VH CDR3 | ARGWDENDY | GWDENDY | WDEND | ARGWDEND | GWDENDY |
| | | (SEQ ID | (SEQ ID | (SEQ ID | (SEQ ID NO: 168) | (SEQ ID |
| | | NO: 103) | NO: 149) | NO: 163) | | NO: 149) |

VH Sequence:
EVKLVESGGDMVKPGGSLKLSCAASGFTFSSYAMSWFRQTSEMRLEWVASITGGGGTYYPDSVKGRFTISRDTVRNILYLQMRSLRSGDTAIYYCARGWDENDYWGQGTTLTVSS (SEQ ID NO: 134)

TABLE 9
Antibody R29-8-30A CDR Sequences

| | | IMGT | Kabat | Chothia | Contact | AbM |
|---|---|---|---|---|---|---|
| VH CDR | VH CDR1 | GFTFSNYA | NYAMS | GFTFSNY | SNYAMS | GFTFSNYAMS |
| Seq. | | (SEQ ID | (SEQ ID | (SEQ ID | (SEQ ID NO: 177) | (SEQ ID |
| | | NO: 89) | NO: 174) | NO: 176) | | NO: 179) |
| | VH CDR2 | ITGGGST | SITGGGSTYY | GGG | WVASITGGGSTY | SITGGGSTY |
| | | (SEQ ID | PDSVKG | (SEQ ID | (SEQ ID NO: 178) | (SEQ ID |
| | | NO: 98) | (SEQ ID | NO: 162) | | NO: 180) |
| | | | NO: 175) | | | |
| | VH CDR3 | ARGWDENDY | GWDENDY | WDEND | ARGWDEND | GWDENDY |
| | | (SEQ ID | (SEQ ID | (SEQ ID | (SEQ ID NO: 168) | (SEQ ID |
| | | NO: 103) | NO: 149) | NO: 163) | | NO: 149) |

VH Sequence:
EVKLVESGGGLVKPGGSLKLSCAASGFTFSNYAMSWFRQTPEKRLEWVASITGGGSTYYPDSVKGRFIISRDNARNILYLQMRSLRSEDTAMYYCARGWDENDYWGQGTTLTVSS (SEQ ID NO: 136)

TABLE 10
Antibody R29-8-120B CDR Sequences

| | | IMGT | Kabat | Chothia | Contact | AbM |
|---|---|---|---|---|---|---|
| VH CDR | VH CDR1 | GFTFSIYA | IYAMS | GFTFSIY | SIYAMS | GFTFSIYAMS |
| Seq. | | (SEQ ID | (SEQ ID | (SEQ ID | (SEQ ID NO: 186) | (SEQ ID |
| | | NO: 95) | NO: 181) | NO: 184) | | NO: 189) |
| | VH CDR2 | ITGGGTN | SITGGGTNFY | GGG | WVASITGGGTNF | SITGGGTNF |
| | | (SEQ ID | PDSVRG | (SEQ ID | (SEQ ID NO: 187) | (SEQ ID |
| | | NO: 99) | (SEQ ID | NO: 162) | | NO: 190) |
| | | | NO: 182) | | | |
| | VH CDR3 | ARGWDENDY | GWDENDY | WDEND | ARGWDEND | GWDENDY |
| | | (SEQ ID | (SEQ ID | (SEQ ID | (SEQ ID NO: 168) | (SEQ ID |
| | | NO: 103) | NO: 149) | NO: 163) | | NO: 149) |
| VL CDR | VL CDR1 | QSLVYSNGNTY | RSSQSLVYSN | SQSLVYSNGNTY | VYSNGNTYLHWY | RSSQSLVYS |
| Seq. | | (SEQ ID | GNTYLH | (SEQ ID | (SEQ ID NO: 188) | NGNTYLH |
| | | NO: 107) | (SEQ ID | NO: 185) | | (SEQ ID |
| | | | NO: 183) | | | NO: 183) |
| | VL CDR2 | KVS | KVSNRFS | KVS | LLIYKVSNRF | KVSNRFS |
| | | (SEQ ID | (SEQ ID | (SEQ ID NO: 71) | (SEQ ID NO: 170) | (SEQ ID |
| | | NO: 71) | NO: 160) | | | NO: 160) |
| | VL CDR3 | SQSTHVPWT | SQSTHVPWT | STHVPW | SQSTHVPW | SQSTHVPWT |
| | | (SEQ ID | (SEQ ID | (SEQ ID | (SEQ ID NO: 171) | (SEQ ID |
| | | NO: 109) | NO: 109) | NO: 165) | | NO: 109) |

VH Sequence: MNFGFSLIFLVLVLKGVQCKVNLVESGGGLVKAGGSLKLSCAASGFTFSIYAMSWFRQTPEKRLEWVASITGGGTNFYPDSVRGRFTISRDNVRNILYLQMSSLRSEDTAMYYCARGWDENDYWGQGTTLTVSS (SEQ ID NO: 52)
VL Sequence: MKLPVRLLVLMFWIPASSSDVVMTQTPLSLPVSLGDQASISCRSSQSLVYSNGNTYLHWYLQKPGQSPKLLIYKVSNRFSGVPDRFSGSGSGTDFTLKISRVEAEDLGVYFCSQSTHVPWTFGGGTKLEIK (SEQ ID NO: 54)

TABLE 11
Antibody R29-8-18B CDR Sequences

| | | IMGT | Kabat | Chothia | Contact | AbM |
|---|---|---|---|---|---|---|
| VH CDR | VH CDR1 | GFTFSTYA | TYAVS | GFTFSTY | STYAVS | GFTFSTYAVS |
| Seq. | | (SEQ ID | (SEQ ID | (SEQ ID | (SEQ ID NO: 195) | (SEQ ID |
| | | NO: 96) | NO: 191) | NO: 193) | | NO: 197) |
| | VH CDR2 | ITSGGST | SITSGGSTYYP | SGG | WVASITSGGSTY | SITSGGSTY |
| | | (SEQ ID | DSVKG | (SEQ ID | (SEQ ID NO: 196) | (SEQ ID |
| | | NO: 100) | (SEQ ID | NO: 194) | | NO: 198) |
| | | | NO: 192) | | | |
| | VH CDR3 | ARGWDENDY | GWDENDY | WDEND | ARGWDEND | GWDENDY |
| | | (SEQ ID | (SEQ ID | (SEQ ID | (SEQ ID NO: 168) | (SEQ ID |
| | | NO: 103) | NO: 149) | NO: 163) | | NO: 149) |

VH Sequence: EVKLVESGGGLVKPGGSLKLSCAASGFTFSTYAVSWFRQTPERRLEWVASITSGGSTYYPDSVKGRFTISRDNARNILYLQMISLRSGDTAMYYCARGWDENDYWGQGTTLTVSS (SEQ ID NO: 138)

TABLE 12
Antibody R29-8-28C CDR Sequences

| | | IMGT | Kabat | Chothia | Contact | AbM |
|---|---|---|---|---|---|---|
| VH CDR | VH CDR1 | GFTFSNYA | NYAVS | GFTFSNY | SNYAVS | GFTFSNYAVS |
| Seq. | | (SEQ ID | (SEQ ID | (SEQ ID | (SEQ ID NO: 202) | (SEQ ID |
| | | NO: 89) | NO: 199) | NO: 176) | | NO: 206) |
| | VH CDR2 | ITSGGRT | SITSGGRTYY | SGG | WVASITSGGRTY | SITSGGRTY |
| | | (SEQ ID | PDSMKG | (SEQ ID | (SEQ ID NO: 203) | (SEQ ID |
| | | NO: 101) | (SEQ ID | NO: 194) | | NO: 207) |
| | | | NO: 200) | | | |
| | VH CDR3 | GRGWDENDY | GWDENDY | WDEND | GRGWDEND | GWDENDY |
| | | (SEQ ID | (SEQ ID | (SEQ ID | (SEQ ID NO: 204) | (SEQ ID |
| | | NO: 104) | NO: 149) | NO: 163) | | NO: 149) |
| VL CDR | VL CDR1 | QSLVYSNGNTY | RSSQSLVYSN | SQSLVYSNGNTY | VYSNGNTYLHWY | RSSQSLVYS |
| Seq. | | (SEQ ID | GNTYLH | (SEQ ID | (SEQ ID NO: 188) | NGNTYLH |
| | | NO: 107) | (SEQ ID | NO: 185) | | (SEQ ID |
| | | | NO: 183) | | | NO: 183) |
| | VL CDR2 | KVS | KVSNRFS | KVS | LLIYKVSNRF | KVSNRFS |
| | | (SEQ ID | (SEQ ID | (SEQ ID NO: 71) | (SEQ ID NO: 170) | (SEQ ID |
| | | NO: 71) | NO: 160) | | | NO: 160) |
| | VL CDR3 | SQTTHVPWT | SQTTHVPWT | TTHVPW | SQTTHVPW | SQTTHVPWT |
| | | (SEQ ID | (SEQ ID | (SEQ ID | (SEQ ID NO: 205) | (SEQ ID |
| | | NO: 110) | NO: 110) | NO: 201) | | NO: 110) |

VH Sequence: MNFGFSLIFLVLVLKGVQSEVKLVESGGGVVKPGGSLTLSCAASGFTFSNYAVSWFRQTPEKRLEWVASITSGGRTYYPDSMKGRFTISRDNARNILYLQMSSLRSEDTAIYYCGRGWDENDYWGQGTTLTVSS (SEQ ID NO: 48)
VL Sequence: MKLPVRLLVLMFWIPASCSDVVMTQTPLSLPVSLGDQASISCRSSQSLVYSNGNTYLHWYLQKPGQSPKLLIYKVSNRFSGVPDRFSGSGSGTDFTLKINRVEAEDLGVYFCSQTTHVPWTFGGGTKLEIK (SEQ ID NO: 50)

TABLE 13
Antibody R29-8-19B CDR Sequences

| | | IMGT | Kabat | Chothia | Contact | AbM |
|---|---|---|---|---|---|---|
| VH CDR | VH CDR1 | GFTFSSYA | SYAVS | GFTFSSY | SSYAVS | GFTFSSYAVS |
| Seq. | | (SEQ ID | (SEQ ID | (SEQ ID | (SEQ ID NO: 211) | (SEQ ID |
| | | NO: 76) | NO: 208) | NO: 161) | | NO: 213) |
| | VH CDR2 | ITSGGRT | SITSGGRTYY | SGG | WVASITSGGRTY | SITSGGRTY |
| | | (SEQ ID | PDSVKG | (SEQ ID | (SEQ ID NO: 203) | (SEQ ID |
| | | NO: 101) | (SEQ ID | NO: 194) | | NO: 207) |
| | | | NO: 209) | | | |
| | VH CDR3 | TRGWDENDL | GWDENDL | WDEND | TRGWDEND | GWDENDL |
| | | (SEQ ID | (SEQ ID | (SEQ ID | (SEQ ID NO: 212) | (SEQ ID |
| | | NO: 105) | NO: 210) | NO: 163) | | NO: 210) |
| VL CDR | VL CDR1 | QSLVYSNGNTY | RSSQSLVYSN | SQSLVYSNGNTY | VYSNGNTYLHWY | RSSQSLVYS |
| Seq. | | (SEQ ID | GNTYLH | (SEQ ID | (SEQ ID NO: 188) | NGNTYLH |
| | | NO: 107) | (SEQ ID | NO: 185) | | (SEQ ID |
| | | | NO: 183) | | | NO: 183) |
| | VL CDR2 | KVS | KVSNRFS | KVS | LLIYKVSNRF | KVSNRFS |
| | | (SEQ ID | (SEQ ID | (SEQ ID NO: 71) | (SEQ ID NO: 170) | (SEQ ID |
| | | NO: 71) | NO: 160) | | | NO: 160) |
| | VL CDR3 | SQTTHVPWT | SQTTHVPWT | TTHVPW | SQTTHVPW | SQTTHVPWT |
| | | (SEQ ID | (SEQ ID | (SEQ ID | (SEQ ID NO: 205) | (SEQ ID |
| | | NO: 110) | NO: 110) | NO: 201) | | NO: 110) |

VH Sequence: MNFGFSLIFLVLVSKGVQCEVKLVESGGGLVKPGGSLKFSCAASGFTFSSYAVSWVRQTPEKRLEWVASITSGGRTYYPDSVKGRFTISRDNARSMLYLHMSSLRSEDTAMYYCTRGWDENDLWGQGTTLTVSS (SEQ ID NO: 36)
VL Sequence: MKLPVRLLVLMFWIPASRSDVVMTQTPLSLPVSLGDQASISCRSSQSLVYSNGNTYLHWYLQKPGQSPQLLIYKVSNRFSGVPDRFSGSGSGTDFTLKISRVEAEDLGVYFCSQTTHVPWTFGGGTKLEIK (SEQ ID NO: 38)

TABLE 14
Antibody R29-8-50C CDR Sequences

| | | IMGT | Kabat | Chothia | Contact | AbM |
|---|---|---|---|---|---|---|
| VH CDR | VH CDR1 | GFTFSSYA | SYAVS | GFTFSSY | SSYAVS | GFTFSSYAVS |
| Seq. | | (SEQ ID | (SEQ ID | (SEQ ID | (SEQ ID NO: 211) | (SEQ ID |
| | | NO: 76) | NO: 208) | NO: 161) | | NO: 213) |
| | VH CDR2 | ITSGGRT | SITSGGRTYY | SGG | WVASITSGGRTY | SITSGGRTY |
| | | (SEQ ID | PDSVKG | (SEQ ID | (SEQ ID NO: 203) | (SEQ ID |
| | | NO: 101) | (SEQ ID | NO: 194) | | NO: 207) |
| | | | NO: 209) | | | |
| | VH CDR3 | TRGWDENDL | GWDENDL | WDEND | TRGWDEND | GWDENDL |
| | | (SEQ ID | (SEQ ID | (SEQ ID | (SEQ ID NO: 212) | (SEQ ID |
| | | NO: 105) | NO: 210) | NO: 163) | | NO: 210) |
| VL CDR | VL CDR1 | QSLVYSNGNTY | RSSQSLVYSN | SQSLVYSNGNTY | VYSNGNTYLHWY | RSSQSLVYS |
| Seq. | | (SEQ ID | GNTYLH | (SEQ ID | (SEQ ID NO: 188) | NGNTYLH |
| | | NO: 107) | (SEQ ID | NO: 185) | | (SEQ ID |
| | | | NO: 183) | | | NO: 183) |
| | VL CDR2 | KVS | KVSNRFS | KVS | LLIYKVSNRF | KVSNRFS |
| | | (SEQ ID | (SEQ ID | (SEQ ID NO: 71) | (SEQ ID NO: 170) | (SEQ ID |
| | | NO: 71) | NO: 160) | | | NO: 160) |
| | VL CDR3 | SQTTHVPWT | SQTTHVPWT | TTHVPW | SQTTHVPW | SQTTHVPWT |
| | | (SEQ ID | (SEQ ID | (SEQ ID | (SEQ ID NO: 205) | (SEQ ID |
| | | NO: 110) | NO: 110) | NO: 201) | | NO: 110) |

VH Sequence: MNFGFSLIFLVLVSKGVQCEVKLVESGGGLVKPGGSLKFSCAASGFTFSSYAVSWVRQTPEKRLEWVASITSGGRTYYPDSVKGRFTISRDNARSMLYLHMSSLRSEDTAMYYCTRGWDENDLWGQGTTLTVSS (SEQ ID NO: 32)
VL Sequence: MKLPVRLLVLMFWIPASSSDVVMTQTPLSLPVSLGDQASISCRSSQSLVYSNGNTYLHWYLQRPGQSPQLLIYKVSNRFSGVPDRFSGSGSGTDFTLKISRVEAEDLGVYFCSQTTHVPWTFGGGTKLEIK (SEQ ID NO: 34)

TABLE 15
Antibody R29-8-12A CDR Sequences

| | | IMGT | Kabat | Chothia | Contact | AbM |
|---|---|---|---|---|---|---|
| VH CDR | VH CDR1 | GFTFSSYA | SYAVS | GFTFSSY | SSYAVS | GFTFSSYAVS |
| Seq. | | (SEQ ID | (SEQ ID | (SEQ ID | (SEQ ID NO: 211) | (SEQ ID |
| | | NO: 76) | NO: 208) | NO: 161) | | NO: 213) |
| | VH CDR2 | ITSGGRT | SITSGGRTYY | SGG | WVASITSGGRTY | SITSGGRTY |
| | | (SEQ ID | PDSVKG | (SEQ ID | (SEQ ID NO: 203) | (SEQ ID |
| | | NO: 101) | (SEQ ID | NO: 194) | | NO: 207) |
| | | | NO: 209) | | | |
| | VH CDR3 | TRGWDENDL | GWDENDL | WDEND | TRGWDEND | GWDENDL |
| | | (SEQ ID | (SEQ ID | (SEQ ID | (SEQ ID NO: 212) | (SEQ ID |
| | | NO: 105) | NO: 210) | NO: 163) | | NO: 210) |
| VL CDR | VL CDR1 | QSLVYSNGNTY | RSSQSLVYSN | SQSLVYSNGNTY | VYSNGNTYLHWY | RSSQSLVYS |
| Seq. | | (SEQ ID | GNTYLH | (SEQ ID | (SEQ ID NO: 188) | NGNTYLH |
| | | NO: 107) | (SEQ ID | NO: 185) | | (SEQ ID |
| | | | NO: 183) | | | NO: 183) |
| | VL CDR2 | KVS | KVSNRFS | KVS | LLIYKVSNRF | KVSNRFS |
| | | (SEQ ID | (SEQ ID | (SEQ ID NO: 71) | (SEQ ID NO: 170) | (SEQ ID |
| | | NO: 71) | NO: 160) | | | NO: 160) |
| | VL CDR3 | SQTTHVPWT | SQTTHVPWT | TTHVPW | SQTTHVPW | SQTTHVPWT |
| | | (SEQ ID | (SEQ ID | (SEQ ID | (SEQ ID NO: 205) | (SEQ ID |
| | | NO: 110) | NO: 110) | NO: 201) | | NO: 110) |

VH Sequence: MNFGFSLIFLVLVSKGVQCEVKLVESGGGLVKPGGSLKFSCAASGFTFSSYAVSWVRQTPEKRLEWVASITSGGRTYYPDSVKGRFTISRDNARSMLYLHMSSLRSEDTAMYYCTRGWDENDLWGQGTTLTVSS (SEQ ID NO: 64)
VL Sequence: MKLPVRLLVLMFWIPASRSDVVMTQTPLSLPVSLGDQASISCRSSQSLVYSNGNTYLHWYLQKPGQSPQLLIYKVSNRFSGVPDRFSGSGSGTDFTLKISRVEAEDLGVYFCSQTTHVPWTFGGGTKLEIK (SEQ ID NO: 66)

TABLE 16
Antibody R29-8-36C CDR Sequences

| | | IMGT | Kabat | Chothia | Contact | AbM |
|---|---|---|---|---|---|---|
| VH CDR | VH CDR1 | GFTFSSYA | SYAVS | GFTFSSY | SSYAVS | GFTFSSYAVS |
| Seq. | | (SEQ ID | (SEQ ID | (SEQ ID | (SEQ ID NO: 211) | (SEQ ID |
| | | NO: 76) | NO: 208) | NO: 161) | | NO: 213) |
| | VH CDR2 | ITSGGRS | SITSGGRSYY | SGG | WVASITSGGRSY | SITSGGRSY |
| | | (SEQ ID | PDSVKG | (SEQ ID | (SEQ ID NO: 164) | (SEQ ID |
| | | NO: 102) | (SEQ ID | NO: 194) | | NO: 218) |
| | | | NO: 214) | | | |
| | VH CDR3 | TRGWDENDL | GWDENDL | WDEND | TRGWDEND | GWDENDL |
| | | (SEQ ID | (SEQ ID | (SEQ ID | (SEQ ID NO: 212) | (SEQ ID |
| | | NO: 105) | NO: 210) | NO: 163) | | NO: 210) |
| VL CDR | VL CDR1 | QSLLYSNGNTY | RSSQSLLYSN | SQSLLYSNGNTY | LYSNGNTYLHWY | RSSQSLLYSN |
| Seq. | | (SEQ ID | GNTYLH | (SEQ ID | (SEQ ID NO: 217) | GNTYLH |
| | | NO: 108) | (SEQ ID | NO: 216) | | (SEQ ID |
| | | | NO: 215) | | | NO: 215) |
| | VL CDR2 | KVS | KVSNRFS | KVS | LLIYKVSNRF | KVSNRFS |
| | | (SEQ ID | (SEQ ID | (SEQ ID NO: 71) | (SEQ ID NO: 170) | (SEQ ID |
| | | NO: 71) | NO: 160) | | | NO: 160) |
| | VL CDR3 | SQTTHVPWT | SQTTHVPWT | TTHVPW | SQTTHVPW | SQTTHVPWT |
| | | (SEQ ID | (SEQ ID | (SEQ ID | (SEQ ID NO: 205) | (SEQ ID |
| | | NO: 110) | NO: 110) | NO: 201) | | NO: 110) |

VH Sequence: MNFGFSLIFLVLVSKGVQCEVKLVESGGGLVKPGGSLKFSCAASGFTFSSYAVSWVRQTPEKRLEWVASITSGGRSYYPDSVKGRFTISRDNARSMLYLHMSSLRSEDTAMYYCTRGWDENDLWGQGTTLTVSS (SEQ ID NO: 60)
VL Sequence: MKLPVRLLVLMFWIPASSSDVVMTQTPLSLFVSLGDQASISCRSSQSLLYSNGNTYLHWYLQRPGQSPQLLIYKVSNRFSGVPDRFSGSGSGTDFTLKISRVEAEDLGVYFCSQTTHVPWTFGGGTKLEIK (SEQ ID NO: 62)

TABLE 17
Antibody R29-8-58C CDR Sequences

| | | IMGT | Kabat | Chothia | Contact | AbM |
|---|---|---|---|---|---|---|
| VH CDR | VH CDR1 | GFTFSSYA | SYAVS | GFTFSSY | SSYAVS | GFTFSSYAVS |
| Seq. | | (SEQ ID | (SEQ ID | (SEQ ID | (SEQ ID NO: 211) | (SEQ ID |
| | | NO: 76) | NO: 208) | NO: 161) | | NO: 213) |
| | VH CDR2 | ITSGGRS | SITSGGRSYY | SGG | WVASITSGGRSY | SITSGGRSY |
| | | (SEQ ID | PDSVKG | (SEQ ID | (SEQ ID NO: 164) | (SEQ ID |
| | | NO: 102) | (SEQ ID | NO: 194) | | NO: 218) |
| | | | NO: 214) | | | |
| | VH CDR3 | TRGWDENDL | GWDENDL | WDEND | TRGWDEND | GWDENDL |
| | | (SEQ ID | (SEQ ID | (SEQ ID | (SEQ ID NO: 212) | (SEQ ID |
| | | NO: 105) | NO: 210) | NO: 163) | | NO: 210) |
| VL CDR | VL CDR1 | QSLLYSNGNTY | RSSQSLLYSN | SQSLLYSNGNTY | LYSNGNTYLHWY | RSSQSLLYSN |
| Seq. | | (SEQ ID | GNTYLH | (SEQ ID | (SEQ ID NO: 217) | GNTYLH |
| | | NO: 108) | (SEQ ID | NO: 216) | | (SEQ ID |
| | | | NO: 215) | | | NO: 215) |
| | VL CDR2 | KVS | KVSNRFS | KVS | LLIYKVSNRF | KVSNRFS |
| | | (SEQ ID | (SEQ ID | (SEQ ID NO: 71) | (SEQ ID NO: 170) | (SEQ ID |
| | | NO: 71) | NO: 160) | | | NO: 160) |
| | VL CDR3 | SQTTHVPWT | SQTTHVPWT | TTHVPW | SQTTHVPW | SQTTHVPWT |
| | | (SEQ ID | (SEQ ID | (SEQ ID | (SEQ ID NO: 205) | (SEQ ID |
| | | NO: 110) | NO: 110) | NO: 201) | | NO: 110) |

VH Sequence: MNFGFSLIFLVLVSKGVQCEVKLVESGGGLVKPGGSLKFSCAASGFTFSSYAVSWVRQTPEKRLEWVASITSGGRSYYPDSVKGRFTISRDNARSMLYLHMSSLRSEDTAMYYCTRGWDENDLWGQGTTLTVSS (SEQ ID NO: 40)
VL Sequence: MKLPVRLLVLMFWIPASSSDVVMTQTPLSLFVSLGDQASISCRSSQSLLYSNGNTYLHWYLQRPGQSPQLLIYKVSNRFSGVPDRFSGSGSGTDFTLKISRVEAEDLGVYFCSQTTHVPWTFGGGTKLEIK (SEQ ID NO: 42)

TABLE 18
Antibody R29-8-75B CDR Sequences

| | | IMGT | Kabat | Chothia | Contact | AbM |
|---|---|---|---|---|---|---|
| VH CDR | VH CDR1 | GFTFSRFG | RFGMH | GFTFSRF | SRFGMH | GFTFSRFGMH |
| Seq. | | (SEQ ID | (SEQ ID | (SEQ ID | (SEQ ID NO: 229) | (SEQ ID |
| | | NO: 73) | NO: 219) | NO: 224) | | NO: 318) |
| | VH CDR2 | ISSGSSTI | YISSGSSTIYY | SGSS | WVAYISSGSSTIY | YISSGSSTIY |
| | | (SEQ ID | ADTVKG | (SEQ ID | (SEQ ID NO: 230) | (SEQ ID |
| | | NO: 74) | (SEQ ID | NO: 225) | | NO: 319) |
| | | | NO: 220) | | | |
| | VH CDR3 | ARVDYDVALAY | VDYDVALAY | DYDVALA | ARVDYDVALA | VDYDVALAY |
| | | (SEQ ID | (SEQ ID | (SEQ ID | (SEQ ID NO: 231) | (SEQ ID |
| | | NO: 75) | NO: 221) | NO: 226) | | NO: 221) |
| VL GDR | VL CDR1 | QSIVHRNGNTY | RSSQSIVHRN | SQSIVHRNGNTY | VHRNGNTYLEWY | RSSQSIVHRN |
| Seq. | | (SEQ ID | GNTYLE | (SEQ ID | (SEQ ID NO: 232) | GNTYLE |
| | | NO: 76) | (SEQ ID | NO: 227) | | (SEQ ID |
| | | | NO: 222) | | | NO: 222) |
| | VL CDR2 | KVS | KVSNRFS | KVS | LLIYKVSNRF | KVSNRFS |
| | | (SEQ ID | (SEQ ID | (SEQ ID NO: 71) | (SEQ ID NO: 170) | (SEQ ID |
| | | NO: 71) | NO: 160) | | | NO: 160) |
| | VL CDR3 | FQGSQWT | FQGSQWT | GSQW | FQGSQW | FQGSQWT |
| | | (SEQ ID | (SEQ ID | (SEQ ID | (SEQ ID NO: 233) | (SEQ ID |
| | | NO: 78) | NO: 78) | NO: 228) | | NO: 78) |

VH Sequence: MDSRLNLVFLVLILKGVQCDVQLVESGGGLVQPGGSRKLSCAASGFTFSRFGMHWVRQAPEKGLEWVAYISSGSSTIYYADTVKGRFTISRDNPKNTLFLQMTSLRSEDTAMYYCARVDYDVALAYWGQGTLVTVSA (SEQ ID NO: 56)
VL Sequence: MKLPVRLLVLMFWIPASSSGVLMTQTPLSLPVSLGDQASISCRSSQSIVHRNGNTYLEWYLQKPGQSPKLLIYKVSNRFSGVPDRFSGSGSGTDFTLKISRVEAEDLGVYYCFQGSQWTFGGGTKLEIK (SEQ ID NO: 58)

TABLE 19
Antibody R29-8-57B CDR Sequences

| | | IMGT | Kabat | Chothia | Contact | AbM |
|---|---|---|---|---|---|---|
| VH CDR | VH CDR1 | GYSITSDYA | SDYAWN | GYSITSDY | TSDYAWN | GYSITSDYAWN |
| Seq. | | (SEQ ID | (SEQ ID | (SEQ ID | (SEQ ID NO: 244) | (SEQ ID |
| | | NO: 111) | NO: 234) | NO: 240) | | NO: 250) |
| | VH CDR2 | INYSGST | YINYSGSTRY | YSG | WMGYINYSGSTR | YINYSGSTR |
| | | (SEQ ID | NPSLKS | (SEQ ID | (SEQ ID NO: 245) | (SEQ ID |
| | | NO: 113) | (SEQ ID | NO: 239) | | NO: 251) |
| | | | NO: 235) | | | |
| | VH CDR3 | ARERYDGVYY | ERYDGVYYG | RYDGVYYGMD | ARERYDGVYYG | ERYDGVYYG |
| | | GMDY | MDY | (SEQ ID | MD | MDY |
| | | (SEQ ID | (SEQ ID | NO: 241) | (SEQ ID NO: 246) | (SEQ ID |
| | | NO: 118) | NO: 236) | | | NO: 236) |
| VL CDR | VL CDR1 | QSLLYSSNQK | KSSQSLLYSS | SQSLLYSSNQK | LYSSNQKNYLA | KSSQSLLYSS |
| Seq. | | NY | NQKNYLA | NY | WY | NQKNYLA |
| | | (SEQ ID | (SEQ ID | (SEQ ID | (SEQ ID NO: 247) | (SEQ ID |
| | | NO: 94) | NO: 237) | NO: 242) | | NO: 237) |
| | VL CDR2 | WAS | WASTRES | WAS | LLIYWASTRE | WASTRES |
| | | (SEQ ID | (SEQ ID | (SEQ ID NO: 83) | (SEQ ID NO: 248) | (SEQ ID |
| | | NO: 83) | NO: 238) | | | NO: 238) |
| | VL CDR3 | QQYYSYRT | QQYYSYRT | YYSYR | QQYYSYR | QQYYSYRT |
| | | (SEQ ID | (SEQ ID | (SEQ ID | (SEQ ID NO: 249) | (SEQ ID |
| | | NO: 124) | NO: 124) | NO: 243) | | NO: 124) |

VH Sequence: MRVLILLWLFTAFPGILSDVQLQESGPGLVKPSQSLSLTCTVTGYSITSDYAWNWIRQFPGNKLEWMGYINYSGSTRYNPSLKSRISITRDTSKNHFFLQLNSVTPEDTATYHCARERYDGVYYGMDYWGQGTSVTVSS (SEQ ID NO: 20)
VL Sequence: MDSQAQVLMSLLLWVSGTCGDIVMSQSPSSLAVSVGEKVTMSCKSSQSLLYSSNQKNYLAWYQQKPGQSPKLLIYWASTRESGVPDRFTGSGSGTDFTLTISSVKTEDLAVYYCQQYYSYRTFGGGTKLEIK (SEQ ID NO: 22)

TABLE 20
Antibody R29-8-136C CDR Sequences

| | | IMGT | Kabat | Chothia | Contact | AbM |
|---|---|---|---|---|---|---|
| VH CDR | VH CDR1 | GYSITSDYA | SDYAWN | GYSITSDY | TSDYAWN | GYSITSDYAWN |
| Seq. | | (SEQ ID | (SEQ ID | (SEQ ID | (SEQ ID NO: 244) | (SEQ ID |
| | | NO: 111) | NO: 234) | NO: 240) | | NO: 250) |
| | VH CDR2 | INYSGSS | YINYSGSSRY | YSG | WMGYINYSGSSR | YINYSGSSR |
| | | (SEQ ID | NPSLKS | (SEQ ID | (SEQ ID NO: 252) | (SEQ ID |
| | | NO: 114) | (SEQ ID | NO: 239) | | NO: 253) |
| | | | NO: 223) | | | |
| | VH CDR3 | ARERYDGVYY | ERYDGVYYG | RYDGVYYGMD | ARERYDGVYYG | ERYDGVYYG |
| | | GMDY | MDY | (SEQ ID | MD | MDY |
| | | (SEQ ID | (SEQ ID | NO: 241) | (SEQ ID NO: 246) | (SEQ ID |
| | | NO: 118) | NO: 236) | | | NO: 236) |
| VL CDR | VL CDR1 | QSLLYSSNQK | KSSQSLLYSS | SQSLLYSSNQK | LYSSNQKNYLA | KSSQSLLYSS |
| Seq. | | NY | NQKNYLA | NY | WY | NQKNYLA |
| | | (SEQ ID | (SEQ ID | (SEQ ID | (SEQ ID NO: 247) | (SEQ ID |
| | | NO: 94) | NO: 237) | NO: 242) | | NO: 237) |
| | VL CDR2 | WAS | WASTRES | WAS | LLIYWASTRE | WASTRES |
| | | (SEQ ID | (SEQ ID | (SEQ ID NO: 83) | (SEQ ID NO: 248) | (SEQ ID |
| | | NO: 83) | NO: 238) | | | NO: 238) |
| | VL CDR3 | QQYYSYRT | QQYYSYRT | YYSYR | QQYYSYR | QQYYSYRT |
| | | (SEQ ID | (SEQ ID | (SEQ ID | (SEQ ID NO: 249) | (SEQ ID |
| | | NO: 124) | NO: 124) | NO: 243) | | NO: 124) |

VH Sequence: MRVLILLWLFTAFPGILSDVQLQESGPGLVKPSQSLSLTCTVTGYSITSDYAWNWIRQFPGNKLEWMGYINYSGSSRYNPSLKSRISITRDTSKNHFFLQLNSVTPEDTATYHCARERYDGVYYGMDYWGQGTSVTVSS (SEQ ID NO: 16)
VL Sequence: MDSQAQVLMSLLLWVSGTCGDIVMSQSPSSLAVSVGEKVTMSCKSSQSLLYSSNQKNYLAWYQQKPGQSPKLLIYWASTRESGVPDRFTGSGSGTDFTLTISSVKTEDLAVYYCQQYYSYRTFGGGTKLEIK (SEQ ID NO: 18)

TABLE 21
Antibody R29-7-1C CDR Sequences

| | | IMGT | Kabat | Chothia | Contact | AbM |
|---|---|---|---|---|---|---|
| VH CDR | VH CDR1 | GYSITSDYA | SDYAWN | GYSITSDY | TSDYAWN | GYSITSDYAWN |
| Seq. | | (SEQ ID | (SEQ ID | (SEQ ID | (SEQ ID NO: 244) | (SEQ ID |
| | | NO: 111) | NO: 234) | NO: 240) | | NO: 250) |
| | VH CDR2 | ISYSGSI | YISYSGSIRYN | YSG | WMGYISYSGSIR | YISYSGSIR |
| | | (SEQ ID | PSLKS | (SEQ ID | (SEQ ID NO: 259) | (SEQ ID |
| | | NO: 115) | (SEQ ID | NO: 239) | | NO: 262) |
| | | | NO: 254) | | | |
| | VH CDR3 | AREKYDNYYY | EKYDNYYYAM | KYDNYYYAMD | AREKYDNYYYA | EKYDNYYYA |
| | | AMDY | DY | (SEQ ID | MD | MDY |
| | | (SEQ ID | (SEQ ID | NO: 257) | (SEQ ID NO: 260) | (SEQ ID |
| | | NO: 119) | NO: 255) | | | NO: 255) |
| VL CDR | VL CDR1 | QNLLYSTNQK | KSSQNLLYST | SQNLLYSTNQK | LYSTNQKNYLA | KSSQNLLYST |
| Seq. | | NY | NQKNYLA | NY | WY | NQKNYLA |
| | | (SEQ ID | (SEQ ID | (SEQ ID | (SEQ ID NO: 261) | (SEQ ID |
| | | NO: 122) | NO: 256) | NO: 258) | | NO: 256) |
| | VL CDR2 | WAS | WASTRES | WAS | LLIYWASTRE | WASTRES |
| | | (SEQ ID | (SEQ ID | (SEQ ID NO: 83) | (SEQ ID NO: 248) | (SEQ ID |
| | | NO: 83) | NO: 238) | | | NO: 238) |
| | VL CDR3 | QQYYSYRT | QQYYSYRT | YYSYR | QQYYSYR | QQYYSYRT |
| | | (SEQ ID | (SEQ ID | (SEQ ID | (SEQ ID NO: 249) | (SEQ ID |
| | | NO: 124) | NO: 124) | NO: 243) | | NO: 124) |

VH Sequence: MRVLILLWLFTAFPGILSDVQLQESGPGLVKPSQSLSLTCTVTGYSITSDYAWNWIRQFPGNKLEWMGYISYSGSIRYNPSLKSRISITRDTSKNQFFLQLNSVTTEDTATYYCAREKYDNYYYAMDYWGQGTSVTVSS (SEQ ID NO: 8)
VL Sequence: MDSQAQVLMLLLLWVSGTCGDIVMSQSPSSLAVSVGEKVTLSCKSSQNLLYSTNQKNYLAWYQQKPGQSPKLLIYWASTRESGVPDRFTGSGSGTDFTLTISSVKAEDLAVYYCQQYYSYRTFGGGTKLEIK (SEQ ID NO: 10)

TABLE 22
Antibody R29-67-7A CDR Sequences

| | | IMGT | Kabat | Chothia | Contact | AbM |
|---|---|---|---|---|---|---|
| VH CDR | VH CDR1 | GYSITSDYA | SDYAWN | GYSITSDY | TSDYAWN | GYSITSDYAWN |
| Seq. | | (SEQ ID | (SEQ ID | (SEQ ID | (SEQ ID NO: 244) | (SEQ ID |
| | | NO: 111) | NO: 234) | NO: 240) | | NO: 250) |
| | VH CDR2 | INYSGRT | YINYSGRTRY | YSG | WMGYINYSGRTR | YINYSGRTR |
| | | (SEQ ID | NPSLNS | (SEQ ID | (SEQ ID NO: 270) | (SEQ ID |
| | | NO: 116) | (SEQ ID | NO: 239) | | NO: 273) |
| | | | NO: 263) | | | |
| | VH CDR3 | ARENYDYEFY | ENYDYEFYAM | NYDYEFYAMD | ARENYDYEFYA | ENYDYEFYA |
| | | AMDY | DY | (SEQ ID | MD | MDY |
| | | (SEQ ID | (SEQ ID | NO: 267) | (SEQ ID NO: 271) | (SEQ ID |
| | | NO: 120) | NO: 264) | | | NO: 264) |
| VL CDR | VL CDR1 | QNLLYSSNQK | KSSQNLLYSS | SQNLLYSSNQK | LYSSNQKNYLA | KSSQNLLYSS |
| Seq. | | NY | NQKNYLA | NY | WY | NQKNYLA |
| | | (SEQ ID | (SEQ ID | (SEQ ID | (SEQ ID NO: 247) | (SEQ ID |
| | | NO: 123) | NO: 265) | NO: 268) | | NO: 265) |
| | VL CDR2 | WAS | WASTRES | WAS | LLIYWASTRE | WASTRES |
| | | (SEQ ID | (SEQ ID | (SEQ ID NO: 83) | (SEQ ID NO: 248) | (SEQ ID |
| | | NO: 83) | NO: 238) | | | NO: 238) |
| | VL CDR3 | QQYYIYRT | QQYYIYRT | YYIYR | QQYYIYR | QQYYIYRT |
| | | (SEQ ID | (SEQ ID | (SEQ ID | (SEQ ID NO: 272) | (SEQ ID |
| | | NO: 125) | NO: 125) | NO: 269) | | NO: 125) |

VH Sequence: MRVLILLWLFTAFPGILSDVQLQESGPGLVKPSQSLSLTCTVTGYSITSDYAWNWIRQFPGNKLEWMGYINYSGRTRYNPSLNSRISLTRDTSTNQFFLQLNSVTTEDTATYYCARENYDYEFYAMDYWGQGTSVTVSS (SEQ ID NO: 12)
VL Sequence: MESQAQVLMLLLLWVSGTCGDIVMSQSPSSLAVSVGEKVTMSCKSSQNLLYSSNQKNYLAWYQQKPGQSPKWYWASTRESGVPDRFTGSGSGTDFTLTISSVKAEDLAVYYCQQYYIYRTFGGGTKLEIK (SEQ ID NO: 14)

TABLE 23
Antibody R29-67-4A CDR Sequences

| | | IMGT | Kabat | Chothia | Contact | AbM |
|---|---|---|---|---|---|---|
| VH CDR | VH CDR1 | GYSITSVYA | SVYAWN | GYSITSVY | TSVYAWN | GYSITSVYAWN |
| Seq. | | (SEQ ID | (SEQ ID | (SEQ ID | (SEQ ID NO: 277) | (SEQ ID |
| | | NO: 112) | NO: 274) | NO: 266) | | NO: 279) |
| | VH CDR2 | ISYSGIT | YISYSGITTYN | YSG | WMGYISYSGITT | YISYSGITT |
| | | (SEQ ID | PSLKS | (SEQ ID | (SEQ ID NO: 278) | (SEQ ID |
| | | NO: 117) | (SEQ ID | NO: 239) | | NO: 280) |
| | | | NO: 275) | | | |
| | VH CDR3 | ATTGTR | TGTR | TGTR | ATTGTR | TGTR |
| | | (SEQ ID | (SEQ ID | (SEQ ID | (SEQ ID NO: 121) | (SEQ ID |
| | | NO: 121) | NO: 276) | NO: 276) | | NO: 276) |

VH Sequence: SDVQLQESGPGLVKPSQSLSLTCTVTGYSITSVYAWNWIRQFPGNKLEWMGYISYSGITTYNPSLKSRISITRDTSKNQFFLQLNSVTTEDTATYYCATTGTRWGQGTTLTVSS (SEQ ID NO: 146)

TABLE 24
Antibody R29-7A-53A CDR Sequences

| | | IMGT | Kabat | Chothia | Contact | AbM |
|---|---|---|---|---|---|---|
| VH CDR | VH CDR1 | GFSLTDYA | DYAIS | GFSLTDY | TDYAIS | GFSLTDYAIS |
| Seq. | | (SEQ ID | (SEQ ID | (SEQ ID | (SEQ ID NO: 289) | (SEQ ID |
| | | NO: 126) | NO: 281) | NO: 285) | | NO: 294) |
| | VH CDR2 | IWGGGRT | VIWGGGRTYS | GGG | WLGVIWGGGRTY | VIWGGGRTY |
| | | (SEQ ID | NSALKS | (SEQ ID | (SEQ ID NO: 290) | (SEQ ID |
| | | NO: 128) | (SEQ ID | NO: 162) | | NO: 295) |
| | | | NO: 282) | | | |
| | VH CDR3 | AKHEEVSRFAY | HEEVSRFAY | EEVSRFA | AKHEEVSRFA | HEEVSRFAY |
| | | (SEQ ID | (SEQ ID | (SEQ ID | (SEQ ID NO: 291) | (SEQ ID |
| | | NO: 130) | NO: 283) | NO: 286) | | NO: 283) |
| VL CDR | VL CDR1 | QSLVYSNGNSY | RSSQSLVYSN | SQSLVYSNGN | VYSNGNSYLHWY | RSSQSLVYS |
| Seq. | | (SEQ ID | GNSYLH | SY | (SEQ ID NO: 292) | NGNSYLH |
| | | NO: 88) | (SEQ ID | (SEQ ID | | (SEQ ID |
| | | | NO: 284) | NO: 287) | | NO: 284) |
| | VL CDR2 | KVS | KVSNRFS | KVS | LLIYKVSNRF | KVSNRFS |
| | | (SEQ ID | (SEQ ID | (SEQ ID NO: 71) | (SEQ ID NO: 170) | (SEQ ID |
| | | NO: 71) | NO: 160) | | | NO: 160) |
| | VL CDR3 | SQSTHIPLT | SQSTHIPLT | STHIPL | SQSTHIPL | SQSTHIPLT |
| | | (SEQ ID | (SEQ ID | (SEQ ID | (SEQ ID NO: 293) | (SEQ ID |
| | | NO: 90) | NO: 90) | NO: 288) | | NO: 90) |

VH Sequence: MAVLGLLLCLVTFPSCVLSQVQLKESGPGLVAPSQSLSITCTVSGFSLTDYAISWIRQPPGKGLEWLGVIWGGGRTYSNSALKSRLSISKDNSRSQIFLKMNSLQTDDTAMYYCAKHEEVSRFAYWGQGTLVTVSV (SEQ ID NO: 28)
VL Sequence: MKLPVRLLVLMFWIPASISDVVLTQTPLSLPVSLGDQASISCRSSQSLVYSNGNSYLHWYLQKPGQSPKLLIYKVSNRFSGVPDRFSGSGSGTDFTLKITRVEAEDLGVYFCSQSTHIPLTFGAGTKLELK (SEQ ID NO: 30)

TABLE 25
Antibody R29-7A-54C CDR Sequences

| | | IMGT | Kabat | Chothia | Contact | AbM |
|---|---|---|---|---|---|---|
| VH CDR | VH CDR1 | GFSLTDYA | DYAIS | GFSLTDY | TDYAIS | GFSLTDYAIS |
| Seq. | | (SEQ ID | (SEQ ID | (SEQ ID | (SEQ ID NO: 289) | (SEQ ID |
| | | NO: 126) | NO: 281) | NO: 285) | | NO: 294) |
| | VH CDR2 | IWGGGRT | VIWGGGRTYS | GGG | WLGVIWGGGRTY | VIWGGGRTY |
| | | (SEQ ID | NSALKS | (SEQ ID | (SEQ ID NO: 290) | (SEQ ID |
| | | NO: 128) | (SEQ ID | NO: 162) | | NO: 295) |
| | | | NO: 282) | | | |
| | VH CDR3 | AKHEEVSRFAY | HEEVSRFAY | EEVSRFA | AKHEEVSRFA | HEEVSRFAY |
| | | (SEQ ID | (SEQ ID | (SEQ ID | (SEQ ID NO: 291) | (SEQ ID |
| | | NO: 130) | NO: 283) | NO: 286) | | NO: 283) |
| VL CDR | VL CDR1 | QSLVYSNGNSY | RSSQSLVYSN | SQSLVYSNGN | VYSNGNSYLHWY | RSSQSLVYS |
| Seq. | | (SEQ ID | GNSYLH | SY | (SEQ ID NO: 292) | NGNSYLH |
| | | NO: 88) | (SEQ ID | (SEQ ID | | (SEQ ID |
| | | | NO: 284) | NO: 287) | | NO: 284) |
| | VL CDR2 | KVS | KVSNRFS | KVS | LLIYKVSNRF | KVSNRFS |
| | | (SEQ ID | (SEQ ID | (SEQ ID NO: 71) | (SEQ ID NO: 170) | (SEQ ID |
| | | NO: 71) | NO: 160) | | | NO: 160) |
| | VL CDR3 | SQSTHIPLT | SQSTHIPLT | STHIPL | SQSTHIPL | SQSTHIPLT |
| | | (SEQ ID | (SEQ ID | (SEQ ID | (SEQ ID NO: 293) | (SEQ ID |
| | | NO: 90) | NO: 90) | NO: 288) | | NO: 90) |

VH Sequence: MAVLGLLLCLVTFPSCVLSQVQLKESGPGLVAPSQSLSITCTVSGFSLTDYAISWIRQPPGKGLEWLGVIWGGGRTYSNSALKSRLSISKDNSRSQIFLKMNSLQTDDTAMYYCAKHEEVSRFAYWGQGTLVTVSV (SEQ ID NO: 24)
VL Sequence: MKLPVRLLVLMFWIPASISDVVLTQTPLSLPVSLGDQASISCRSSQSLVYSNGNSYLHWYLQKPGQSPKLLIYKVSNRFSGVPDRFSGSGSGTDFTLKITRVEAEDLGVYFCSQSTHIPLTFGAGTKLELK (SEQ ID NO: 26)

TABLE 26
Antibody R29-7A-38C CDR Sequences

| | | IMGT | Kabat | Chothia | Contact | AbM |
|---|---|---|---|---|---|---|
| VH CDR | VH CDR1 | GFSLTDYA | DYAIS | GFSLTDY | TDYAIS | GFSLTDYAIS |
| Seq. | | (SEQ ID | (SEQ ID | (SEQ ID | (SEQ ID NO: 289) | (SEQ ID |
| | | NO: 126) | NO: 281) | NO: 285) | | NO: 294) |
| | VH CDR2 | IWGGGRT | VIWGGGRTYS | GGG | WLGVIWGGGRTY | VIWGGGRTY |
| | | (SEQ ID | NSALKS | (SEQ ID | (SEQ ID NO: 290) | (SEQ ID |
| | | NO: 128) | (SEQ ID | NO: 162) | | NO: 295) |
| | | | NO: 282) | | | |
| | VH CDR3 | AKHEEVSRFAH | HEEVSRFAH | EEVSRFA | AKHEEVSRFA | HEEVSRFAH |
| | | (SEQ ID | (SEQ ID | (SEQ ID | (SEQ ID NO: 291) | (SEQ ID |
| | | NO: 77) | NO: 296) | NO: 286) | | NO: 296) |

VH Sequence: QVQLKESGPGLVAPSQSLSITCTVSGFSLTDYAISWIRQPPGKGLEWLGVIWGGGRTYSNSALKSRLSISKDNSRSQIFLKMNSLQTDDTAMYYCAKHEEVSRFAHWGQGTLVTVSA (SEQ ID NO: 140)

TABLE 27
Antibody R29-7A-49A CDR Sequences

| | | IMGT | Kabat | Chothia | Contact | AbM |
|---|---|---|---|---|---|---|
| VH CDR | VH CDR1 | GFSLTDYG | DYGVS | GFSLTDY | TDYGVS | GFSLTDYGVS |
| Seq. | | (SEQ ID | (SEQ ID | (SEQ ID | (SEQ ID NO: 299) | (SEQ ID |
| | | NO: 127) | NO: 297) | NO: 285) | | NO: 301) |
| | VH CDR2 | IWGGGGT | VIWGGGGTYY | GGG | WLGVIWGGGGTY | VIWGGGGTY |
| | | (SEQ ID | NSALKS | (SEQ ID | (SEQ ID NO: 300) | (SEQ ID |
| | | NO: 129) | (SEQ ID | NO: 162) | | NO: 302) |
| | | | NO: 298) | | | |
| | VH CDR3 | AKHEEVSRFAY | HEEVSRFAY | EEVSRFA | AKHEEVSRFA | HEEVSRFAY |
| | | (SEQ ID | (SEQ ID | (SEQ ID | (SEQ ID NO: 291) | (SEQ ID |
| | | NO: 130) | NO: 283) | NO: 286) | | NO: 283) |

VH Sequence: QVQLKESGPGLVAPSQSLSITCTVSGFSLTDYGVSWIRQPPGKGLEWLGVIWGGGGTYYNSALKSRLSINKDNSKSQIFLKMNSLQTDDTAIYYCAKHEEVSRFAYWGQGTLVTVSA (SEQ ID NO: 142)

TABLE 28
Antibody R29-7A-13A CDR Sequences

| | | IMGT | Kabat | Chothia | Contact | AbM |
|---|---|---|---|---|---|---|
| VH CDR | VH CDR1 | GFSLTDYA | DYAIS | GFSLTDY | TDYAIS | GFSLTDYAIS |
| Seq. | | (SEQ ID | (SEQ ID | (SEQ ID | (SEQ ID NO: 289) | (SEQ ID |
| | | NO: 126) | NO: 281) | NO: 285) | | NO: 294) |
| | VH CDR2 | IWGGGRT | VIWGGGRTYS | GGG | WLGVIWGGGRTY | VIWGGGRTY |
| | | (SEQ ID | NSALKS | (SEQ ID | (SEQ ID NO: 290) | (SEQ ID |
| | | NO: 128) | (SEQ ID | NO: 162) | | NO: 295) |
| | | | NO: 282) | | | |
| | VH CDR3 | AKHEEVSRFAY | HEEVSRFAY | EEVSRFA | AKHEEVSRFA | HEEVSRFAY |
| | | (SEQ ID | (SEQ ID | (SEQ ID | (SEQ ID NO: 291) | (SEQ ID |
| | | NO: 130) | NO: 283) | NO: 286) | | NO: 283) |

VH Sequence: QVQLKESGPGLVAPSQSLSITCTVSGFSLTDYAISWIRQPPGKGLEWLGVIWGGGRTYSNSALKSRLSISKDNSRSQIFLKMNSLHTEDTAVYYCAKHEEVSRFAYWGQGTLVTVSA (SEQ ID NO: 144)

TABLE 29
Antibody R29-7-2A CDR Sequences

| | | IMGT | Kabat | Chothia | Contact | AbM |
|---|---|---|---|---|---|---|
| VH CDR | VH CDR1 | GFSFNTHA | THAMN | GFSFNTH | NTHAMN | GFSFNTHAMN |
| Seq. | | (SEQ ID | (SEQ ID | (SEQ ID | (SEQ ID NO: 311) | (SEQ ID |
| | | NO: 91) | NO: 303) | NO: 307) | | NO: 317) |
| | VH CDR2 | IRSKSNNYAR | RIRSKSNNYA | SKSNNY | WVARIRSKSNN | RIRSKSNNYA |
| | | (SEQ ID | RYYADSVKD | (SEQ ID | YARY | RY |
| | | NO: 92) | (SEQ ID | NO: 308) | (SEQ ID NO: 312) | (SEQ ID |
| | | | NO: 304) | | | NO: 316) |
| | VH CDR3 | VKQGDGGFAY | QGDGGFAY | GDGGFA | VKQGDGGFA | QGDGGFAY |
| | | (SEQ ID | (SEQ ID | (SEQ ID | (SEQ ID NO: 313) | (SEQ ID |
| | | NO: 93) | NO: 305) | NO: 309) | | NO: 305) |
| VL GDR | VL CDR1 | QSLLYSSNQK | KSSQSLLYSS | SQSLLYSSNQK | LYSSNQKNYLA | KSSQSLLYSS |
| Seq. | | NY | NQKNYLA | NY | WY | NQKNYLA |
| | | (SEQ ID | (SEQ ID | (SEQ ID | (SEQ ID NO: 247) | (SEQ ID |
| | | NO: 94) | NO: 237) | NO: 242) | | NO: 237) |
| | VL CDR2 | WAS | WASARES | WAS | LLIYWASARE | WASARES |
| | | (SEQ ID | (SEQ ID | (SEQ ID NO: 83) | (SEQ ID NO: 314) | (SEQ ID |
| | | NO: 83) | NO: 306) | | | NO: 306) |
| | VL CDR3 | QQYYSYPPT | QQYYSYPPT | YYSYPP | QQYYSYPP | QQYYSYPPT |
| | | (SEQ ID | (SEQ ID | (SEQ ID | (SEQ ID NO: 315) | (SEQ ID |
| | | NO: 96) | NO: 96) | NO: 310) | | NO: 96) |

VH Sequence: MLLGLKWVFFVVFYQGVPCEVQLVESGGGLIQPKGSLKLSCVASGFSFNTHAMNWVRQAPGKGLEWVARIRSKSNNYARYYADSVKDRFTISRDDSQSILYLQMNNLKTEDTAMYYCVKQGDGGFAYWGQGTLVTVSA (SEQ ID NO: 4)
VL Sequence: MDSQAQVLMLLLLWVSGTCGDIVMSQSPSSLAVSVGEKVTMSCKSSQSLLYSSNQKNYLAWYQQRPGQSPKLLIYWASARESGVPDRFTGSGSGTDFTLTISSVKAEDLAVYYCQQYYSYPPTFGAGTKVELK (SEQ ID NO: 6)

In some embodiments, the antibodies provided herein comprise a VH region that comprises or consists of a VH domain. In other embodiments, the antibodies provided herein comprise a VH region that comprises or consists of a VH chain. In some embodiments, the antibodies provided herein comprise a VL region that comprises or consists of a VL domain. In other embodiments, the antibodies provided herein comprise a VL region that comprises or consists of a VL chain. In some embodiments, the antibodies provided herein have a combination of (i) a VH domain or VH chain; and/or (ii) a VL domain or VL chain.

In some embodiments, an antibody provided herein comprises or consists of six CDRs, for example, VH CDR1, VH CDR2, VH CDR3, VL CDR1, VL CDR2, and/or VL CDR3 identified in Tables 1-29. In certain embodiments, an antibody provided herein can comprise less than six CDRs. In some embodiments, the antibody comprises or consists of one, two, three, four, or five CDRs selected from the group consisting of VH CDR1, VH CDR2, VH CDR3, VL CDR1, VL CDR2, and/or VL CDR3. In specific embodiments, the antibody comprises or consists of one, two, three, four, or five CDRs selected from the group consisting of VH CDR1, VH CDR2, VH CDR3, VL CDR1, VL CDR2, and/or VL CDR3 of the murine monoclonal antibody selected from the group consisting of: (a) the antibody designated R29-7-2A; (b) the antibody designated R29-7-1C; (c) the antibody designated R29-67-7A; (d) the antibody designated R29-8-136C; (e) the antibody designated R29-8-57B; (f) the antibody designated R29-7-54C; (g) the antibody designated R29-7-53A; (h) the antibody designated R29-8-50C; (i) the antibody designated R29-8-19B; (j) the antibody designated R29-8-58C; (k) the antibody designated R29-8-9B; (l) the antibody designated R29-8-28C; (m) the antibody designated R29-8-120B; (n) the antibody designated R29-8-75B; (o) the antibody designated R29-8-36C; (p) the antibody designated R29-8-12A; (q) the antibody designated R29-8-93B; (r) the antibody designated R29-8-51B; (s) the antibody designated R29-8-30A; (t) the antibody designated R29-8-18B; (u) the antibody designated R29-7-38C; (v) the antibody designated R29-7-49A; (w) the antibody designated R29-7-13A; or (x) the antibody designated R29-67-4A described herein. Accordingly, in some embodiments, the antibody comprises or consists of one, two, three four or five CDRs of anyone of the VH CDR1, VH CDR2, VH CDR3, VL CDR1, VL CDR2, and/or VL CDR3 identified in Tables 1-29.

In some embodiments, the antibodies provided herein comprise one or more (eg. one, two or three) VH CDRs listed in Tables 1-29. In other embodiments, the antibodies provided herein comprise one or more (eg. one, two or three) VL CDRs listed in Tables 1-6, 10, 12-22, 24, 25 and 29. In yet other embodiments, the antibodies provided herein comprise one or more (eg. one, two or three) VH CDRs listed in Tables 1-29 and one or more VL CDRs listed in Tables 1-6, 10, 12-22, 24, 25 and 29. Accordingly, in certain embodiments, the antibodies comprise a VH CDR1 having the amino acid sequence of any one of SEQ ID NOS: 67, 73, 76, 79, 85, 89, 91, 95, 96, 111, 112, 126, 127, 147, 161, 166, 172, 174, 176, 177, 179, 181, 184, 186, 189, 191, 193, 195, 197, 199, 202, 206, 208, 211, 213, 219, 224, 229, 234, 240, 244, 250, 266, 274, 277, 279, 281, 285, 289, 294, 297, 299, 301, 303, 307, 311, 317, 318. In another embodiment, the antibodies comprise a VH CDR2 having the amino acid sequence of any one of SEQ ID NOS: 68, 74, 80, 86, 92, 97, 98, 99, 100, 101, 102, 114, 115, 116, 117, 128, 129, 148, 162, 164, 167, 173, 175, 178, 180, 182, 187, 190, 192, 194, 196, 198, 203, 207, 209, 214, 218, 220, 223, 225, 230, 239, 252, 253, 254, 259, 262, 263, 270, 273, 275, 278, 280, 282, 290, 295, 298, 300, 302, 304, 308, 312, 316, 319. In another embodiment, the antibodies comprise a VH CDR3 having the amino acid sequence of any one of SEQ ID NOS: 69, 75, 77, 81, 87, 93, 101, 103, 104, 105, 118, 119, 120, 121, 130, 149, 163, 168, 194, 200, 203, 204, 207, 210, 212, 226, 231, 236, 241, 246, 255, 257, 260, 264, 267, 271, 276, 283, 286, 291, 296, 305, 309, 313. In certain embodiments, the antibodies comprise a VH CDR1 and/or a VH CDR2 and/or a VH CDR3 independently selected from a VH CDR1, VH CDR2, VH CDR3 as depicted in any one of the amino acid sequences depicted in Table 1-29. In certain embodiments, the antibodies comprise a VL CDR1 having the amino acid sequence of any one of SEQ ID NOS: 70, 76, 82, 88, 94, 106, 107, 108, 122, 123, 150, 164, 169, 183, 185, 188, 215, 216, 217, 222, 227, 232, 237, 242, 247, 256, 258, 261, 265, 268, 284, 287, 292. In another embodiment, the antibodies comprise a VL CDR2 having the amino acid sequence of any one of SEQ ID NOS: 71, 83, 160, 170, 238, 248, 306, 314. In another embodiment, the antibodies comprise a VL CDR3 having the amino acid sequence of any one of SEQ ID NOS: 72, 78, 84, 90, 96, 109, 110, 124, 125, 165, 171, 201, 205, 228, 233, 243, 249, 269, 272, 288, 293, 310, 315. In certain embodiments, the antibodies comprise a VL CDR1 and/or a VL CDR2 and/or a VL CDR3 independently selected from a VL CDR1, VL CDR2, VL CDR3 as depicted in any one of the amino acid sequences depicted in Tables 1-6, 10, 12-22, 24, 25 and 29.

Also provided herein are antibodies comprising one or more VH CDRs and one or more (eg. one, two or three) VL CDRs listed in Tables 1-29. In particular, provided herein is an antibody comprising a VH CDR1 (SEQ ID NOS: 67, 73, 76, 79, 85, 89, 91, 95, 96, 111, 112, 126, 127, 147, 161, 166, 172, 174, 176, 177, 179, 181, 184, 186, 189, 191, 193, 195, 197, 199, 202, 206, 208, 211, 213, 219, 224, 229, 234, 240, 244, 250, 266, 274, 277, 279, 281, 285, 289, 294, 297, 299, 301, 303, 307, 311, 317, 318) and a VL CDR1 (SEQ ID NOS: 70, 76, 82, 88, 94, 106, 107, 108, 122, 123, 150, 164, 169, 183, 185, 188, 215, 216, 217, 222, 227, 232, 237, 242, 247, 256, 258, 261, 265, 268, 284, 287, 292); a VH CDR1 (SEQ ID NOS: 67, 73, 76, 79, 85, 89, 91, 95, 96, 111, 112, 126, 127, 147, 161, 166, 172, 174, 176, 177, 179, 181, 184, 186, 189, 191, 193, 195, 197, 199, 202, 206, 208, 211, 213, 219, 224, 229, 234, 240, 244, 250, 266, 274, 277, 279, 281, 285, 289, 294, 297, 299, 301, 303, 307, 311, 317, 318) and a VL CDR2 (SEQ ID NOS: 71, 83, 160, 170, 238, 248, 306, 314); a VH CDR1 (SEQ ID NOS: 67, 73, 76, 79, 85, 89, 91, 95, 96, 111, 112, 126, 127, 147, 161, 166, 172, 174, 176, 177, 179, 181, 184, 186, 189, 191, 193, 195, 197, 199, 202, 206, 208, 211, 213, 219, 224, 229, 234, 240, 244, 250, 266, 274, 277, 279, 281, 285, 289, 294, 297, 299, 301, 303, 307, 311, 317, 318) and a VL CDR3 (SEQ ID NOS: 72, 78, 84, 90, 96, 109, 110, 124, 125, 165, 171, 201, 205, 228, 233, 243, 249, 269, 272, 288, 293, 310, 315); a VH CDR2 (SEQ ID NOS: 68, 74, 80, 86, 92, 97, 98, 99, 100, 101, 102, 114, 115, 116, 117, 128, 129, 148, 162, 164, 167, 173, 175, 178, 180, 182, 187, 190, 192, 194, 196, 198, 203, 207, 209, 214, 218, 220, 223, 225, 230, 239, 252, 253, 254, 259, 262, 263, 270, 273, 275, 278, 280, 282, 290, 295, 298, 300, 302, 304, 308, 312, 316, 319) and a VL CDR1 (SEQ ID NOS: 70, 76, 82, 88, 94, 106, 107, 108, 122, 123, 150, 164, 169, 183, 185, 188, 215, 216, 217, 222, 227, 232, 237, 242, 247, 256, 258, 261, 265, 268, 284, 287, 292); a VH CDR2 (SEQ ID NOS: 68, 74, 80, 86, 92, 97, 98, 99, 100, 101, 102, 114, 115, 116, 117, 128, 129, 148, 162, 164, 167, 173, 175, 178, 180, 182, 187, 190, 192, 194, 196, 198, 203, 207, 209, 214, 218, 220, 223, 225, 230, 239, 252, 253, 254, 259, 262, 263, 270, 273, 275, 278, 280, 282, 290, 295, 298, 300, 302, 304, 308, 312, 316, 319) and a VL CDR2 (SEQ ID NOS: 71, 83, 160, 170, 238, 248, 306, 314); a VH CDR2 (SEQ ID NOS: 68, 74, 80, 86, 92, 97, 98, 99, 100, 101, 102, 114, 115, 116, 117, 128, 129, 148, 162, 164, 167, 173, 175, 178, 180, 182, 187, 190, 192, 194, 196, 198, 203, 207, 209, 214, 218, 220, 223, 225, 230, 239, 252, 253, 254, 259, 262, 263, 270, 273, 275, 278, 280, 282, 290, 295, 298, 300, 302, 304, 308, 312, 316, 319) and a VL CDR3 (SEQ ID NOS: 72, 78, 84, 90, 96, 109, 110, 124, 125, 165, 171, 201, 205, 228, 233, 243, 249, 269, 272, 288, 293, 310, 315); a VH CDR3 (SEQ ID NOS: 69, 75, 77, 81, 87, 93, 101, 103, 104, 105, 118, 119, 120, 121, 130, 149, 163, 168, 194, 200, 203, 204, 207, 210, 212, 226, 231, 236, 241, 246, 255, 257, 260, 264, 267, 271, 276, 283, 286, 291, 296, 305, 309, 313) and a VL CDR1 (SEQ ID NOS: 70, 76, 82, 88, 94, 106, 107, 108, 122, 123, 150, 164, 169, 183, 185, 188, 215, 216, 217, 222, 227, 232, 237, 242, 247, 256, 258, 261, 265, 268, 284, 287, 292); a VH CDR3 (SEQ ID NOS: 69, 75, 77, 81, 87, 93, 101, 103, 104, 105, 118, 119, 120, 121, 130, 149, 163, 168, 194, 200, 203, 204, 207, 210, 212, 226, 231, 236, 241, 246, 255, 257, 260, 264, 267, 271, 276, 283, 286, 291, 296, 305, 309, 313) and a VL CDR2 (SEQ ID NOS: 71, 83, 160, 170, 238, 248, 306, 314); a VH CDR3 (SEQ ID NOS: 69, 75, 77, 81, 87, 93, 101, 103, 104, 105, 118, 119, 120, 121, 130, 149, 163, 168, 194, 200, 203, 204, 207, 210, 212, 226, 231, 236, 241, 246, 255, 257, 260, 264, 267, 271, 276, 283, 286, 291, 296, 305, 309, 313) and a VL CDR3 (SEQ ID NOS: 72, 78, 84, 90, 96, 109, 110, 124, 125, 165, 171, 201, 205, 228, 233, 243, 249, 269, 272, 288, 293, 310, 315); a VH CDR1 (SEQ ID NOS: 67, 73, 76, 79, 85, 89, 91, 95, 96, 111, 112, 126, 127, 147, 161, 166, 172, 174, 176, 177, 179, 181, 184, 186, 189, 191, 193, 195, 197, 199, 202, 206, 208, 211, 213, 219, 224, 229, 234, 240, 244, 250, 266, 274, 277, 279, 281, 285, 289, 294, 297, 299, 301, 303, 307, 311, 317, 318), a VH CDR2 (SEQ ID NOS: 68, 74, 80, 86, 92, 97, 98, 99, 100, 101, 102, 114, 115, 116, 117, 128, 129, 148, 162, 164, 167, 173, 175, 178, 180, 182, 187, 190, 192, 194, 196, 198, 203, 207, 209, 214, 218, 220, 223, 225, 230, 239, 252, 253, 254, 259, 262, 263, 270, 273, 275, 278, 280, 282, 290, 295, 298, 300, 302, 304, 308, 312, 316, 319) and a VL CDR1 (SEQ ID NOS: 70, 76, 82, 88, 94, 106, 107, 108, 122, 123, 150, 164, 169, 183, 185, 188, 215, 216, 217, 222, 227, 232, 237, 242, 247, 256, 258, 261, 265, 268, 284, 287, 292); a VH CDR1 (SEQ ID NOS: 67, 73, 76, 79, 85, 89, 91, 95, 96, 111, 112, 126, 127, 147, 161, 166, 172, 174, 176, 177, 179, 181, 184, 186, 189, 191, 193, 195, 197, 199, 202, 206, 208, 211, 213, 219, 224, 229, 234, 240, 244, 250, 266, 274, 277, 279, 281, 285, 289, 294, 297, 299, 301, 303, 307, 311, 317, 318), a VH CDR2 (SEQ ID NOS: 68, 74, 80, 86, 92, 97, 98, 99, 100, 101, 102, 114, 115, 116, 117, 128, 129, 148, 162, 164, 167, 173, 175, 178, 180, 182, 187, 190, 192, 194, 196, 198, 203, 207, 209, 214, 218, 220, 223, 225, 230, 239, 252, 253, 254, 259, 262, 263, 270, 273, 275, 278, 280, 282, 290, 295, 298, 300, 302, 304, 308, 312, 316, 319) and a VL CDR2 (SEQ ID NOS: 71, 83, 160, 170, 238, 248, 306, 314); a VH CDR1 (SEQ ID NOS: 67, 73, 76, 79, 85, 89, 91, 95, 96, 111, 112, 126, 127, 147, 161, 166, 172, 174, 176, 177, 179, 181, 184, 186, 189, 191, 193, 195, 197, 199, 202, 206, 208, 211, 213, 219, 224, 229, 234, 240, 244, 250, 266, 274, 277, 279, 281, 285, 289, 294, 297, 299, 301, 303, 307, 311, 317, 318), a VH CDR2 (SEQ ID NOS: 68, 74, 80, 86, 92, 97, 98, 99, 100, 101, 102, 114, 115, 116, 117, 128, 129, 148, 162, 164, 167, 173, 175, 178, 180, 182, 187, 190, 192, 194, 196, 198, 203, 207, 209, 214, 218, 220, 223, 225, 230, 239, 252, 253, 254, 259, 262, 263, 270, 273, 275, 278, 280, 282, 290, 295, 298, 300, 302, 304, 308, 312, 316, 319) and a VL CDR3 (SEQ ID NOS: 72, 78, 84, 90, 96, 109, 110, 124, 125, 165, 171, 201, 205, 228, 233, 243, 249, 269, 272, 288, 293, 310, 315);a VH CDR2 (SEQ ID NOS: 68, 74, 80, 86, 92, 97, 98, 99, 100, 101, 102, 114, 115, 116, 117, 128, 129, 148, 162, 164, 167, 173, 175, 178, 180, 182, 187, 190, 192, 194, 196, 198, 203, 207, 209, 214, 218, 220, 223, 225, 230, 239, 252, 253, 254, 259, 262, 263, 270, 273, 275, 278, 280, 282, 290, 295, 298, 300, 302, 304, 308, 312, 316, 319), a VH CDR3 (SEQ ID NOS: 69, 75, 77, 81, 87, 93, 101, 103, 104, 105, 118, 119, 120, 121, 130, 149, 163, 168, 194, 200, 203, 204, 207, 210, 212, 226, 231, 236, 241, 246, 255, 257, 260, 264, 267, 271, 276, 283, 286, 291, 296, 305, 309, 313) and a VL CDR1 (SEQ ID NOS: 70, 76, 82, 88, 94, 106, 107, 108, 122, 123, 150, 164, 169, 183, 185, 188, 215, 216, 217, 222, 227, 232, 237, 242, 247, 256, 258, 261, 265, 268, 284, 287, 292), a VH CDR2 (SEQ ID NOS: 68, 74, 80, 86, 92, 97, 98, 99, 100, 101, 102, 114, 115, 116, 117, 128, 129, 148, 162, 164, 167, 173, 175, 178, 180, 182, 187, 190, 192, 194, 196, 198, 203, 207, 209, 214, 218, 220, 223, 225, 230, 239, 252, 253, 254, 259, 262, 263, 270, 273, 275, 278, 280, 282, 290, 295, 298, 300, 302, 304, 308, 312, 316, 319), a VH CDR3 (SEQ ID NOS: 69, 75, 77, 81, 87, 93, 101, 103, 104, 105, 118, 119, 120, 121, 130, 149, 163, 168, 194, 200, 203, 204, 207, 210, 212, 226, 231, 236, 241, 246, 255, 257, 260, 264, 267, 271, 276, 283, 286, 291, 296, 305, 309, 313) and a VL CDR2 (SEQ ID NOS: 71, 83, 160, 170, 238, 248, 306, 314); a VH CDR2 (SEQ ID NOS: 68, 74, 80, 86, 92, 97, 98, 99, 100, 101, 102, 114, 115, 116, 117, 128, 129, 148, 162, 164, 167, 173, 175, 178, 180, 182, 187, 190, 192, 194, 196, 198, 203, 207, 209, 214, 218, 220, 223, 225, 230, 239, 252, 253, 254, 259, 262, 263, 270, 273, 275, 278, 280, 282, 290, 295, 298, 300, 302, 304, 308, 312, 316, 319), a VH CDR3 (SEQ ID NOS: 69, 75, 77, 81, 87, 93, 101, 103, 104, 105, 118, 119, 120, 121, 130, 149, 163, 168, 194, 200, 203, 204, 207, 210, 212, 226, 231, 236, 241, 246, 255, 257, 260, 264, 267, 271, 276, 283, 286, 291, 296, 305, 309, 313) and a VL CDR3 (SEQ ID NOS: 72, 78, 84, 90, 96, 109, 110, 124, 125, 165, 171, 201, 205, 228, 233, 243, 249, 269, 272, 288, 293, 310, 315); a VH CDR1 (SEQ ID NOS: 67, 73, 76, 79, 85, 89, 91, 95, 96, 111, 112, 126, 127, 147, 161, 166, 172, 174, 176, 177, 179, 181, 184, 186, 189, 191, 193, 195, 197, 199, 202, 206, 208, 211, 213, 219, 224, 229, 234, 240, 244, 250, 266, 274, 277, 279, 281, 285, 289, 294, 297, 299, 301, 303, 307, 311, 317, 318), a VL CDR1 (SEQ ID NOS: 70, 76, 82, 88, 94, 106, 107, 108, 122, 123, 150, 164, 169, 183, 185, 188, 215, 216, 217, 222, 227, 232, 237, 242, 247, 256, 258, 261, 265, 268, 284, 287, 292) and a VL CDR2 (SEQ ID NOS: 71, 83, 160, 170, 238, 248, 306, 314); a VH CDR1 (SEQ ID NOS: 67, 73, 76, 79, 85, 89, 91, 95, 96, 111, 112, 126, 127, 147, 161, 166, 172, 174, 176, 177, 179, 181, 184, 186, 189, 191, 193, 195, 197, 199, 202, 206, 208, 211, 213, 219, 224, 229, 234, 240, 244, 250, 266, 274, 277, 279, 281, 285, 289, 294, 297, 299, 301, 303, 307, 311, 317, 318), a VL CDR1 (SEQ ID NOS: 70, 76, 82, 88, 94, 106, 107, 108, 122, 123, 150, 164, 169, 183, 185, 188, 215, 216, 217, 222, 227, 232, 237, 242, 247, 256, 258, 261, 265, 268, 284, 287, 292) and a VL CDR3 (SEQ ID NOS: 72, 78, 84, 90, 96, 109, 110, 124, 125, 165, 171, 201, 205, 228, 233, 243, 249, 269, 272, 288, 293, 310, 315); a VH CDR1 (SEQ ID NOS: 67, 73, 76, 79, 85, 89, 91, 95, 96, 111, 112, 126, 127, 147, 161, 166, 172, 174, 176, 177, 179, 181, 184, 186, 189, 191, 193, 195, 197, 199, 202, 206, 208, 211, 213, 219, 224, 229, 234, 240, 244, 250, 266, 274, 277, 279, 281, 285, 289, 294, 297, 299, 301, 303, 307, 311, 317, 318), a VL CDR2 (SEQ ID NOS: 71, 83, 160, 170, 238, 248, 306, 314) and a VL CDR3 (SEQ ID NOS: 72, 78, 84, 90, 96, 109, 110, 124, 125, 165, 171, 201, 205, 228, 233, 243, 249, 269, 272, 288, 293, 310, 315); a VH CDR2 (SEQ ID NOS: 68, 74, 80, 86, 92, 97, 98, 99, 100, 101, 102, 114, 115, 116, 117, 128, 129, 148, 162, 164, 167, 173, 175, 178, 180, 182, 187, 190, 192, 194, 196, 198, 203, 207, 209, 214, 218, 220, 223, 225, 230, 239, 252, 253, 254, 259, 262, 263, 270, 273, 275, 278, 280, 282, 290, 295, 298, 300, 302, 304, 308, 312, 316, 319), a VL CDR1 (SEQ ID NOS: 70, 76, 82, 88, 94, 106, 107, 108, 122, 123, 150, 164, 169, 183, 185, 188, 215, 216, 217, 222, 227, 232, 237, 242, 247, 256, 258, 261, 265, 268, 284, 287, 292) and a VL CDR2 (SEQ ID NOS: 71, 83, 160, 170, 238, 248, 306, 314); a VH CDR2 (SEQ ID NOS: 68, 74, 80, 86, 92, 97, 98, 99, 100, 101, 102, 114, 115, 116, 117, 128, 129, 148, 162, 164, 167, 173, 175, 178, 180, 182, 187, 190, 192, 194, 196, 198, 203, 207, 209, 214, 218, 220, 223, 225, 230, 239, 252, 253, 254, 259, 262, 263, 270, 273, 275, 278, 280, 282, 290, 295, 298, 300, 302, 304, 308, 312, 316, 319), a VL CDR1 (SEQ ID NOS: 70, 76, 82, 88, 94, 106, 107, 108, 122, 123, 150, 164, 169, 183, 185, 188, 215, 216, 217, 222, 227, 232, 237, 242, 247, 256, 258, 261, 265, 268, 284, 287, 292) and a VL CDR3 (SEQ ID NOS: 72, 78, 84, 90, 96, 109, 110, 124, 125, 165, 171, 201, 205, 228, 233, 243, 249, 269, 272, 288, 293, 310, 315); a VH CDR2 (SEQ ID NOS: 68, 74, 80, 86, 92, 97, 98, 99, 100, 101, 102, 114, 115, 116, 117, 128, 129, 148, 162, 164, 167, 173, 175, 178, 180, 182, 187, 190, 192, 194, 196, 198, 203, 207, 209, 214, 218, 220, 223, 225, 230, 239, 252, 253, 254, 259, 262, 263, 270, 273, 275, 278, 280, 282, 290, 295, 298, 300, 302, 304, 308, 312, 316, 319), a VL CDR2 (SEQ ID NOS: 71, 83, 160, 170, 238, 248, 306, 314) and a VL CDR3 (SEQ ID NOS: 72, 78, 84, 90, 96, 109, 110, 124, 125, 165, 171, 201, 205, 228, 233, 243, 249, 269, 272, 288, 293, 310, 315); a VH CDR3 (SEQ ID NOS: 69, 75, 77, 81, 87, 93, 101, 103, 104, 105, 118, 119, 120, 121, 130, 149, 163, 168, 194, 200, 203, 204, 207, 210, 212, 226, 231, 236, 241, 246, 255, 257, 260, 264, 267, 271, 276, 283, 286, 291, 296, 305, 309, 313), a VL CDR1 (SEQ ID NOS: 70, 76, 82, 88, 94, 106, 107, 108, 122, 123, 150, 164, 169, 183, 185, 188, 215, 216, 217, 222, 227, 232, 237, 242, 247, 256, 258, 261, 265, 268, 284, 287, 292) and a VL CDR2 (SEQ ID NOS: 71, 83, 160, 170, 238, 248, 306, 314); a VH CDR3 (SEQ ID NOS: 69, 75, 77, 81, 87, 93, 101, 103, 104, 105, 118, 119, 120, 121, 130, 149, 163, 168, 194, 200, 203, 204, 207, 210, 212, 226, 231, 236, 241, 246, 255, 257, 260, 264, 267, 271, 276, 283, 286, 291, 296, 305, 309, 313), a VL CDR1 (SEQ ID NOS: 70, 76, 82, 88, 94, 106, 107, 108, 122, 123, 150, 164, 169, 183, 185, 188, 215, 216, 217, 222, 227, 232, 237, 242, 247, 256, 258, 261, 265, 268, 284, 287, 292) and a VL CDR3 (SEQ ID NOS: 72, 78, 84, 90, 96, 109, 110, 124, 125, 165, 171, 201, 205, 228, 233, 243, 249, 269, 272, 288, 293, 310, 315); a VH CDR3 (SEQ ID NOS: 69, 75, 77, 81, 87, 93, 101, 103, 104, 105, 118, 119, 120, 121, 130, 149, 163, 168, 194, 200, 203, 204, 207, 210, 212, 226, 231, 236, 241, 246, 255, 257, 260, 264, 267, 271, 276, 283, 286, 291, 296, 305, 309, 313), a VL CDR2 (SEQ ID NOS: 71, 83, 160, 170, 238, 248, 306, 314) and a VL CDR3 (SEQ ID NOS: 72, 78, 84, 90, 96, 109, 110, 124, 125, 165, 171, 201, 205, 228, 233, 243, 249, 269, 272, 288, 293, 310, 315); a VH CDR1 (SEQ ID NOS: 67, 73, 76, 79, 85, 89, 91, 95, 96, 111, 112, 126, 127, 147, 161, 166, 172, 174, 176, 177, 179, 181, 184, 186, 189, 191, 193, 195, 197, 199, 202, 206, 208, 211, 213, 219, 224, 229, 234, 240, 244, 250, 266, 274, 277, 279, 281, 285, 289, 294, 297, 299, 301, 303, 307, 311, 317, 318), a VH CDR2 (SEQ ID NOS: 68, 74, 80, 86, 92, 97, 98, 99, 100, 101, 102, 114, 115, 116, 117, 128, 129, 148, 162, 164, 167, 173, 175, 178, 180, 182, 187, 190, 192, 194, 196, 198, 203, 207, 209, 214, 218, 220, 223, 225, 230, 239, 252, 253, 254, 259, 262, 263, 270, 273, 275, 278, 280, 282, 290, 295, 298, 300, 302, 304, 308, 312, 316, 319), a VH CDR3 (SEQ ID NOS: 69, 75, 77, 81, 87, 93, 101, 103, 104, 105, 118, 119, 120, 121, 130, 149, 163, 168, 194, 200, 203, 204, 207, 210, 212, 226, 231, 236, 241, 246, 255, 257, 260, 264, 267, 271, 276, 283, 286, 291, 296, 305, 309, 313) and a VL CDR1 (SEQ ID NOS: 70, 76, 82, 88, 94, 106, 107, 108, 122, 123, 150, 164, 169, 183, 185, 188, 215, 216, 217, 222, 227, 232, 237, 242, 247, 256, 258, 261, 265, 268, 284, 287, 292); a VH CDR1 (SEQ ID NOS: 67, 73, 76, 79, 85, 89, 91, 95, 96, 111, 112, 126, 127, 147, 161, 166, 172, 174, 176, 177, 179, 181, 184, 186, 189, 191, 193, 195, 197, 199, 202, 206, 208, 211, 213, 219, 224, 229, 234, 240, 244, 250, 266, 274, 277, 279, 281, 285, 289, 294, 297, 299, 301, 303, 307, 311, 317, 318), a VH CDR2 (SEQ ID NOS: 68, 74, 80, 86, 92, 97, 98, 99, 100, 101, 102, 114, 115, 116, 117, 128, 129, 148, 162, 164, 167, 173, 175, 178, 180, 182, 187, 190, 192, 194, 196, 198, 203, 207, 209, 214, 218, 220, 223, 225, 230, 239, 252, 253, 254, 259, 262, 263, 270, 273, 275, 278, 280, 282, 290, 295, 298, 300, 302, 304, 308, 312, 316, 319), a VH CDR3 (SEQ ID NOS: 69, 75, 77, 81, 87, 93, 101, 103, 104, 105, 118, 119, 120, 121, 130, 149, 163, 168, 194, 200, 203, 204, 207, 210, 212, 226, 231, 236, 241, 246, 255, 257, 260, 264, 267, 271, 276, 283, 286, 291, 296, 305, 309, 313) and a VL CDR2 (SEQ ID NOS: 71, 83, 160, 170, 238, 248, 306, 314); a VH CDR1 (SEQ ID NOS: 67, 73, 76, 79, 85, 89, 91, 95, 96, 111, 112, 126, 127, 147, 161, 166, 172, 174, 176, 177, 179, 181, 184, 186, 189, 191, 193, 195, 197, 199, 202, 206, 208, 211, 213, 219, 224, 229, 234, 240, 244, 250, 266, 274, 277, 279, 281, 285, 289, 294, 297, 299, 301, 303, 307, 311, 317, 318), a VH CDR2 (SEQ ID NOS: 68, 74, 80, 86, 92, 97, 98, 99, 100, 101, 102, 114, 115, 116, 117, 128, 129, 148, 162, 164, 167, 173, 175, 178, 180, 182, 187, 190, 192, 194, 196, 198, 203, 207, 209, 214, 218, 220, 223, 225, 230, 239, 252, 253, 254, 259, 262, 263, 270, 273, 275, 278, 280, 282, 290, 295, 298, 300, 302, 304, 308, 312, 316, 319), a VH CDR3 (SEQ ID NOS: 69, 75, 77, 81, 87, 93, 101, 103, 104, 105, 118, 119, 120, 121, 130, 149, 163, 168, 194, 200, 203, 204, 207, 210, 212, 226, 231, 236, 241, 246, 255, 257, 260, 264, 267, 271, 276, 283, 286, 291, 296, 305, 309, 313) and a VL CDR3 (SEQ ID NOS: 72, 78, 84, 90, 96, 109, 110, 124, 125, 165, 171, 201, 205, 228, 233, 243, 249, 269, 272, 288, 293, 310, 315); a VH CDR1 (SEQ ID NOS: 67, 73, 76, 79, 85, 89, 91, 95, 96, 111, 112, 126, 127, 147, 161, 166, 172, 174, 176, 177, 179, 181, 184, 186, 189, 191, 193, 195, 197, 199, 202, 206, 208, 211, 213, 219, 224, 229, 234, 240, 244, 250, 266, 274, 277, 279, 281, 285, 289, 294, 297, 299, 301, 303, 307, 311, 317, 318), a VH CDR2 (SEQ ID NOS: 68, 74, 80, 86, 92, 97, 98, 99, 100, 101, 102, 114, 115, 116, 117, 128, 129, 148, 162, 164, 167, 173, 175, 178, 180, 182, 187, 190, 192, 194, 196, 198, 203, 207, 209, 214, 218, 220, 223, 225, 230, 239, 252, 253, 254, 259, 262, 263, 270, 273, 275, 278, 280, 282, 290, 295, 298, 300, 302, 304, 308, 312, 316, 319), a VL CDR1 (SEQ ID NOS: 70, 76, 82, 88, 94, 106, 107, 108, 122, 123, 150, 164, 169, 183, 185, 188, 215, 216, 217, 222, 227, 232, 237, 242, 247, 256, 258, 261, 265, 268, 284, 287, 292) and a VL CDR2 (SEQ ID NOS: 71, 83, 160, 170, 238, 248, 306, 314); a VH CDR1 (SEQ ID NOS: 67, 73, 76, 79, 85, 89, 91, 95, 96, 111, 112, 126, 127, 147, 161, 166, 172, 174, 176, 177, 179, 181, 184, 186, 189, 191, 193, 195, 197, 199, 202, 206, 208, 211, 213, 219, 224, 229, 234, 240, 244, 250, 266, 274, 277, 279, 281, 285, 289, 294, 297, 299, 301, 303, 307, 311, 317, 318), a VH CDR2 (SEQ ID NOS: 68, 74, 80, 86, 92, 97, 98, 99, 100, 101, 102, 114, 115, 116, 117, 128, 129, 148, 162, 164, 167, 173, 175, 178, 180, 182, 187, 190, 192, 194, 196, 198, 203, 207, 209, 214, 218, 220, 223, 225, 230, 239, 252, 253, 254, 259, 262, 263, 270, 273, 275, 278, 280, 282, 290, 295, 298, 300, 302, 304, 308, 312, 316, 319), a VL CDR1 (SEQ ID NOS: 70, 76, 82, 88, 94, 106, 107, 108, 122, 123, 150, 164, 169, 183, 185, 188, 215, 216, 217, 222, 227, 232, 237, 242, 247, 256, 258, 261, 265, 268, 284, 287, 292) and a VL CDR3 (SEQ ID NOS: 72, 78, 84, 90, 96, 109, 110, 124, 125, 165, 171, 201, 205, 228, 233, 243, 249, 269, 272, 288, 293, 310, 315); a VH CDR1 (SEQ ID NOS: 67, 73, 76, 79, 85, 89, 91, 95, 96, 111, 112, 126, 127, 147, 161, 166, 172, 174, 176, 177, 179, 181, 184, 186, 189, 191, 193, 195, 197, 199, 202, 206, 208, 211, 213, 219, 224, 229, 234, 240, 244, 250, 266, 274, 277, 279, 281, 285, 289, 294, 297, 299, 301, 303, 307, 311, 317, 318), a VH CDR2 (SEQ ID NOS: 68, 74, 80, 86, 92, 97, 98, 99, 100, 101, 102, 114, 115, 116, 117, 128, 129, 148, 162, 164, 167, 173, 175, 178, 180, 182, 187, 190, 192, 194, 196, 198, 203, 207, 209, 214, 218, 220, 223, 225, 230, 239, 252, 253, 254, 259, 262, 263, 270, 273, 275, 278, 280, 282, 290, 295, 298, 300, 302, 304, 308, 312, 316, 319), a VL CDR2 (SEQ ID NOS: 71, 83, 160, 170, 238, 248, 306, 314) and a VL CDR3 (SEQ ID NOS: 72, 78, 84, 90, 96, 109, 110, 124, 125, 165, 171, 201, 205, 228, 233, 243, 249, 269, 272, 288, 293, 310, 315); a VH CDR1 (SEQ ID NOS: 67, 73, 76, 79, 85, 89, 91, 95, 96, 111, 112, 126, 127, 147, 161, 166, 172, 174, 176, 177, 179, 181, 184, 186, 189, 191, 193, 195, 197, 199, 202, 206, 208, 211, 213, 219, 224, 229, 234, 240, 244, 250, 266, 274, 277, 279, 281, 285, 289, 294, 297, 299, 301, 303, 307, 311, 317, 318), a VH CDR3 (SEQ ID NOS: 69, 75, 77, 81, 87, 93, 101, 103, 104, 105, 118, 119, 120, 121, 130, 149, 163, 168, 194, 200, 203, 204, 207, 210, 212, 226, 231, 236, 241, 246, 255, 257, 260, 264, 267, 271, 276, 283, 286, 291, 296, 305, 309, 313), a VL CDR1 (SEQ ID NOS: 70, 76, 82, 88, 94, 106, 107, 108, 122, 123, 150, 164, 169, 183, 185, 188, 215, 216, 217, 222, 227, 232, 237, 242, 247, 256, 258, 261, 265, 268, 284, 287, 292) and a VL CDR2 (SEQ ID NOS: 71, 83, 160, 170, 238, 248, 306, 314); a VH CDR1 (SEQ ID NOS: 67, 73, 76, 79, 85, 89, 91, 95, 96, 111, 112, 126, 127, 147, 161, 166, 172, 174, 176, 177, 179, 181, 184, 186, 189, 191, 193, 195, 197, 199, 202, 206, 208, 211, 213, 219, 224, 229, 234, 240, 244, 250, 266, 274, 277, 279, 281, 285, 289, 294, 297, 299, 301, 303, 307, 311, 317, 318), a VH CDR3 (SEQ ID NOS: 69, 75, 77, 81, 87, 93, 101, 103, 104, 105, 118, 119, 120, 121, 130, 149, 163, 168, 194, 200, 203, 204, 207, 210, 212, 226, 231, 236, 241, 246, 255, 257, 260, 264, 267, 271, 276, 283, 286, 291, 296, 305, 309, 313), a VL CDR1 (SEQ ID NOS: 70, 76, 82, 88, 94, 106, 107, 108, 122, 123, 150, 164, 169, 183, 185, 188, 215, 216, 217, 222, 227, 232, 237, 242, 247, 256, 258, 261, 265, 268, 284, 287, 292) and a VL CDR3 (SEQ ID NOS: 72, 78, 84, 90, 96, 109, 110, 124, 125, 165, 171, 201, 205, 228, 233, 243, 249, 269, 272, 288, 293, 310, 315); a VH CDR1 (SEQ ID NOS: 67, 73, 76, 79, 85, 89, 91, 95, 96, 111, 112, 126, 127, 147, 161, 166, 172, 174, 176, 177, 179, 181, 184, 186, 189, 191, 193, 195, 197, 199, 202, 206, 208, 211, 213, 219, 224, 229, 234, 240, 244, 250, 266, 274, 277, 279, 281, 285, 289, 294, 297, 299, 301, 303, 307, 311, 317, 318), a VH CDR3 (SEQ ID NOS: 69, 75, 77, 81, 87, 93, 101, 103, 104, 105, 118, 119, 120, 121, 130, 149, 163, 168, 194, 200, 203, 204, 207, 210, 212, 226, 231, 236, 241, 246, 255, 257, 260, 264, 267, 271, 276, 283, 286, 291, 296, 305, 309, 313), a VL CDR2 (SEQ ID NOS: 71, 83, 160, 170, 238, 248, 306, 314) and a VL CDR3 (SEQ ID NOS: 72, 78, 84, 90, 96, 109, 110, 124, 125, 165, 171, 201, 205, 228, 233, 243, 249, 269, 272, 288, 293, 310, 315); a VH CDR2 (SEQ ID NOS: 68, 74, 80, 86, 92, 97, 98, 99, 100, 101, 102, 114, 115, 116, 117, 128, 129, 148, 162, 164, 167, 173, 175, 178, 180, 182, 187, 190, 192, 194, 196, 198, 203, 207, 209, 214, 218, 220, 223, 225, 230, 239, 252, 253, 254, 259, 262, 263, 270, 273, 275, 278, 280, 282, 290, 295, 298, 300, 302, 304, 308, 312, 316, 319), a VH CDR3 (SEQ ID NOS: 69, 75, 77, 81, 87, 93, 101, 103, 104, 105, 118, 119, 120, 121, 130, 149, 163, 168, 194, 200, 203, 204, 207, 210, 212, 226, 231, 236, 241, 246, 255, 257, 260, 264, 267, 271, 276, 283, 286, 291, 296, 305, 309, 313), a VL CDR1 (SEQ ID NOS: 70, 76, 82, 88, 94, 106, 107, 108, 122, 123, 150, 164, 169, 183, 185, 188, 215, 216, 217, 222, 227, 232, 237, 242, 247, 256, 258, 261, 265, 268, 284, 287, 292) and a VL CDR2 (SEQ ID NOS: 71, 83, 160, 170, 238, 248, 306, 314); a VH CDR2 (SEQ ID NOS: 68, 74, 80, 86, 92, 97, 98, 99, 100, 101, 102, 114, 115, 116, 117, 128, 129, 148, 162, 164, 167, 173, 175, 178, 180, 182, 187, 190, 192, 194, 196, 198, 203, 207, 209, 214, 218, 220, 223, 225, 230, 239, 252, 253, 254, 259, 262, 263, 270, 273, 275, 278, 280, 282, 290, 295, 298, 300, 302, 304, 308, 312, 316, 319), a VH CDR3 (SEQ ID NOS: 69, 75, 77, 81, 87, 93, 101, 103, 104, 105, 118, 119, 120, 121, 130, 149, 163, 168, 194, 200, 203, 204, 207, 210, 212, 226, 231, 236, 241, 246, 255, 257, 260, 264, 267, 271, 276, 283, 286, 291, 296, 305, 309, 313), a VL CDR1 (SEQ ID NOS: 70, 76, 82, 88, 94, 106, 107, 108, 122, 123, 150, 164, 169, 183, 185, 188, 215, 216, 217, 222, 227, 232, 237, 242, 247, 256, 258, 261, 265, 268, 284, 287, 292) and a VL CDR3 (SEQ ID NOS: 72, 78, 84, 90, 96, 109, 110, 124, 125, 165, 171, 201, 205, 228, 233, 243, 249, 269, 272, 288, 293, 310, 315); a VH CDR2 (SEQ ID NOS: 68, 74, 80, 86, 92, 97, 98, 99, 100, 101, 102, 114, 115, 116, 117, 128, 129, 148, 162, 164, 167, 173, 175, 178, 180, 182, 187, 190, 192, 194, 196, 198, 203, 207, 209, 214, 218, 220, 223, 225, 230, 239, 252, 253, 254, 259, 262, 263, 270, 273, 275, 278, 280, 282, 290, 295, 298, 300, 302, 304, 308, 312, 316, 319), a VH CDR3 (SEQ ID NOS: 69, 75, 77, 81, 87, 93, 101, 103, 104, 105, 118, 119, 120, 121, 130, 149, 163, 168, 194, 200, 203, 204, 207, 210, 212, 226, 231, 236, 241, 246, 255, 257, 260, 264, 267, 271, 276, 283, 286, 291, 296, 305, 309, 313), a VL CDR2 (SEQ ID NOS: 71, 83, 160, 170, 238, 248, 306, 314) and a VL CDR3 (SEQ ID NOS: 72, 78, 84, 90, 96, 109, 110, 124, 125, 165, 171, 201, 205, 228, 233, 243, 249, 269, 272, 288, 293, 310, 315); a VH CDR1 (SEQ ID NOS: 67, 73, 76, 79, 85, 89, 91, 95, 96, 111, 112, 126, 127, 147, 161, 166, 172, 174, 176, 177, 179, 181, 184, 186, 189, 191, 193, 195, 197, 199, 202, 206, 208, 211, 213, 219, 224, 229, 234, 240, 244, 250, 266, 274, 277, 279, 281, 285, 289, 294, 297, 299, 301, 303, 307, 311, 317, 318), a VH CDR2 (SEQ ID NOS: 68, 74, 80, 86, 92, 97, 98, 99, 100, 101, 102, 114, 115, 116, 117, 128, 129, 148, 162, 164, 167, 173, 175, 178, 180, 182, 187, 190, 192, 194, 196, 198, 203, 207, 209, 214, 218, 220, 223, 225, 230, 239, 252, 253, 254, 259, 262, 263, 270, 273, 275, 278, 280, 282, 290, 295, 298, 300, 302, 304, 308, 312, 316, 319), a VH CDR3 (SEQ ID NOS: 69, 75, 77, 81, 87, 93, 101, 103, 104, 105, 118, 119, 120, 121, 130, 149, 163, 168, 194, 200, 203, 204, 207, 210, 212, 226, 231, 236, 241, 246, 255, 257, 260, 264, 267, 271, 276, 283, 286, 291, 296, 305, 309, 313), a VL CDR1 (SEQ ID NOS: 70, 76, 82, 88, 94, 106, 107, 108, 122, 123, 150, 164, 169, 183, 185, 188, 215, 216, 217, 222, 227, 232, 237, 242, 247, 256, 258, 261, 265, 268, 284, 287, 292) and a VL CDR2 (SEQ ID NOS: 71, 83, 160, 170, 238, 248, 306, 314); a VH CDR1 (SEQ ID NOS: 67, 73, 76, 79, 85, 89, 91, 95, 96, 111, 112, 126, 127, 147, 161, 166, 172, 174, 176, 177, 179, 181, 184, 186, 189, 191, 193, 195, 197, 199, 202, 206, 208, 211, 213, 219, 224, 229, 234, 240, 244, 250, 266, 274, 277, 279, 281, 285, 289, 294, 297, 299, 301, 303, 307, 311, 317, 318), a VH CDR2 (SEQ ID NOS: 68, 74, 80, 86, 92, 97, 98, 99, 100, 101, 102, 114, 115, 116, 117, 128, 129, 148, 162, 164, 167, 173, 175, 178, 180, 182, 187, 190, 192, 194, 196, 198, 203, 207, 209, 214, 218, 220, 223, 225, 230, 239, 252, 253, 254, 259, 262, 263, 270, 273, 275, 278, 280, 282, 290, 295, 298, 300, 302, 304, 308, 312, 316, 319), a VH CDR3 (SEQ ID NOS: 69, 75, 77, 81, 87, 93, 101, 103, 104, 105, 118, 119, 120, 121, 130, 149, 163, 168, 194, 200, 203, 204, 207, 210, 212, 226, 231, 236, 241, 246, 255, 257, 260, 264, 267, 271, 276, 283, 286, 291, 296, 305, 309, 313), a VL CDR1 (SEQ ID NOS: 70, 76, 82, 88, 94, 106, 107, 108, 122, 123, 150, 164, 169, 183, 185, 188, 215, 216, 217, 222, 227, 232, 237, 242, 247, 256, 258, 261, 265, 268, 284, 287, 292) and a VL CDR3 (SEQ ID NOS: 72, 78, 84, 90, 96, 109, 110, 124, 125, 165, 171, 201, 205, 228, 233, 243, 249, 269, 272, 288, 293, 310, 315); a VH CDR1 (SEQ ID NOS: 67, 73, 76, 79, 85, 89, 91, 95, 96, 111, 112, 126, 127, 147, 161, 166, 172, 174, 176, 177, 179, 181, 184, 186, 189, 191, 193, 195, 197, 199, 202, 206, 208, 211, 213, 219, 224, 229, 234, 240, 244, 250, 266, 274, 277, 279, 281, 285, 289, 294, 297, 299, 301, 303, 307, 311, 317, 318), a VH CDR2 (SEQ ID NOS: 68, 74, 80, 86, 92, 97, 98, 99, 100, 101, 102, 114, 115, 116, 117, 128, 129, 148, 162, 164, 167, 173, 175, 178, 180, 182, 187, 190, 192, 194, 196, 198, 203, 207, 209, 214, 218, 220, 223, 225, 230, 239, 252, 253, 254, 259, 262, 263, 270, 273, 275, 278, 280, 282, 290, 295, 298, 300, 302, 304, 308, 312, 316, 319), a VH CDR3 (SEQ ID NOS: 69, 75, 77, 81, 87, 93, 101, 103, 104, 105, 118, 119, 120, 121, 130, 149, 163, 168, 194, 200, 203, 204, 207, 210, 212, 226, 231, 236, 241, 246, 255, 257, 260, 264, 267, 271, 276, 283, 286, 291, 296, 305, 309, 313), a VL CDR2 (SEQ ID NOS: 71, 83, 160, 170, 238, 248, 306, 314) and a VL CDR3 (SEQ ID NOS: 72, 78, 84, 90, 96, 109, 110, 124, 125, 165, 171, 201, 205, 228, 233, 243, 249, 269, 272, 288, 293, 310, 315); a VH CDR1 (SEQ ID NOS: 67, 73, 76, 79, 85, 89, 91, 95, 96, 111, 112, 126, 127, 147, 161, 166, 172, 174, 176, 177, 179, 181, 184, 186, 189, 191, 193, 195, 197, 199, 202, 206, 208, 211, 213, 219, 224, 229, 234, 240, 244, 250, 266, 274, 277, 279, 281, 285, 289, 294, 297, 299, 301, 303, 307, 311, 317, 318), a VH CDR2 (SEQ ID NOS: 68, 74, 80, 86, 92, 97, 98, 99, 100, 101, 102, 114, 115, 116, 117, 128, 129, 148, 162, 164, 167, 173, 175, 178, 180, 182, 187, 190, 192, 194, 196, 198, 203, 207, 209, 214, 218, 220, 223, 225, 230, 239, 252, 253, 254, 259, 262, 263, 270, 273, 275, 278, 280, 282, 290, 295, 298, 300, 302, 304, 308, 312, 316, 319), a VL CDR1 (SEQ ID NOS: 70, 76, 82, 88, 94, 106, 107, 108, 122, 123, 150, 164, 169, 183, 185, 188, 215, 216, 217, 222, 227, 232, 237, 242, 247, 256, 258, 261, 265, 268, 284, 287, 292), a VL CDR2 (SEQ ID NOS: 71, 83, 160, 170, 238, 248, 306, 314), and a VL CDR3 (SEQ ID NOS: 72, 78, 84, 90, 96, 109, 110, 124, 125, 165, 171, 201, 205, 228, 233, 243, 249, 269, 272, 288, 293, 310, 315); a VH CDR1 (SEQ ID NOS: 67, 73, 76, 79, 85, 89, 91, 95, 96, 111, 112, 126, 127, 147, 161, 166, 172, 174, 176, 177, 179, 181, 184, 186, 189, 191, 193, 195, 197, 199, 202, 206, 208, 211, 213, 219, 224, 229, 234, 240, 244, 250, 266, 274, 277, 279, 281, 285, 289, 294, 297, 299, 301, 303, 307, 311, 317, 318), a VH CDR3 (SEQ ID NOS: 69, 75, 77, 81, 87, 93, 101, 103, 104, 105, 118, 119, 120, 121, 130, 149, 163, 168, 194, 200, 203, 204, 207, 210, 212, 226, 231, 236, 241, 246, 255, 257, 260, 264, 267, 271, 276, 283, 286, 291, 296, 305, 309, 313), a VL CDR1 (SEQ ID NOS: 70, 76, 82, 88, 94, 106, 107, 108, 122, 123, 150, 164, 169, 183, 185, 188, 215, 216, 217, 222, 227, 232, 237, 242, 247, 256, 258, 261, 265, 268, 284, 287, 292), a VL CDR2 (SEQ ID NOS: 71, 83, 160, 170, 238, 248, 306, 314), and a VL CDR3 (SEQ ID NOS: 72, 78, 84, 90, 96, 109, 110, 124, 125, 165, 171, 201, 205, 228, 233, 243, 249, 269, 272, 288, 293, 310, 315); a VH CDR2 (SEQ ID NOS: 68, 74, 80, 86, 92, 97, 98, 99, 100, 101, 102, 114, 115, 116, 117, 128, 129, 148, 162, 164, 167, 173, 175, 178, 180, 182, 187, 190, 192, 194, 196, 198, 203, 207, 209, 214, 218, 220, 223, 225, 230, 239, 252, 253, 254, 259, 262, 263, 270, 273, 275, 278, 280, 282, 290, 295, 298, 300, 302, 304, 308, 312, 316, 319), a VH CDR3 (SEQ ID NOS: 69, 75, 77, 81, 87, 93, 101, 103, 104, 105, 118, 119, 120, 121, 130, 149, 163, 168, 194, 200, 203, 204, 207, 210, 212, 226, 231, 236, 241, 246, 255, 257, 260, 264, 267, 271, 276, 283, 286, 291, 296, 305, 309, 313), a VL CDR1 (SEQ ID NOS: 70, 76, 82, 88, 94, 106, 107, 108, 122, 123, 150, 164, 169, 183, 185, 188, 215, 216, 217, 222, 227, 232, 237, 242, 247, 256, 258, 261, 265, 268, 284, 287, 292), a VL CDR2 (SEQ ID NOS: 71, 83, 160, 170, 238, 248, 306, 314), and a VL CDR3 (SEQ ID NOS: 72, 78, 84, 90, 96, 109, 110, 124, 125, 165, 171, 201, 205, 228, 233, 243, 249, 269, 272, 288, 293, 310, 315); or any combination thereof of the VH CDRs (SEQ ID NOS:36, 30, 59-62, 33, 37, 101-104, 50, 114, 99, 100, 31, 65-74, 83-90, 95, 321, 322, 835-842, 34, 847-858, 49, 886-894 1070-1078, 38, 32, 319, 35 or 883-885) and VL CDRs (SEQ ID NOS:45, 253-271, 42, 46, 272-275, 40, 843-846, 43, 1045-1048, 47, 41, 44 or 1056-1058) listed in Tables 1-29.

1. Polyclonal Antibodies

The antibodies of the invention may comprise polyclonal antibodies. Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include the C16orf54 polypeptide or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation. The mammal can then be bled, and the serum assayed for C16orf54 antibody titer. If desired, the mammal can be boosted until the antibody titer increases or plateaus.

2. Monoclonal Antibodies

The antibodies of the invention may alternatively be monoclonal antibodies. Monoclonal antibodies may be made using the hybridoma method first described by Kohler et al., Nature, 256:495 (1975), or may be made by recombinant DNA methods (see, e.g., U.S. Pat. No. 4,816,567).

In the hybridoma method, a mouse or other appropriate host animal, such as a hamster, is immunized as described above to elicit lymphocytes that produce or are capable of producing antibodies that will specifically bind to the protein used for immunization. Alternatively, lymphocytes may be immunized in vitro. After immunization, lymphocytes are isolated and then fused with a myeloma cell line using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium which medium preferably contains one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically will include hypoxanthine, aminopterin, and thymidine (HAT medium), which substances prevent the growth of HGPRT-deficient cells.

Preferred fusion partner myeloma cells are those that fuse efficiently, support stable high-level production of antibody by the selected antibody-producing cells, and are sensitive to a selective medium that selects against the unfused parental cells. Preferred myeloma cell lines are murine myeloma lines, such as those derived from MOPC-21 and MPC-11 mouse tumors available from the Salk Institute Cell Distribution Center, San Diego, Calif. USA, and SP-2 and derivatives e.g., X63-Ag8-653 cells available from the American Type Culture Collection, Manassas, Va., USA. Human myeloma and mouse-human heteromyeloma cell lines also have been described for the production of human monoclonal antibodies (Kozbor, J., Immunol., 133:3001 (1984); and Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA). The binding affinity of the monoclonal antibody can, for example, be determined by the Scatchard analysis described in Munson et al., Anal. Biochem., 107:220 (1980).

Once hybridoma cells that produce antibodies of the desired specificity, affinity, and/or activity are identified, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal e.g., by i.p. injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-Sepharose) or ion-exchange chromatography, hydroxylapatite chromatography, gel electrophoresis, dialysis, etc.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Plückthun, Immunol. Revs. 130:151-188 (1992).

In a specific embodiment, an antibody that binds a C16orf54 epitope comprises an amino acid sequence of a VH domain and/or an amino acid sequence of a VL domain encoded by a nucleotide sequence that hybridizes to (1) the complement of a nucleotide sequence encoding any one of the VH and/or VL domain depicted herein under stringent conditions (e.g., hybridization to filter-bound DNA in 6× sodium chloride/sodium citrate (SSC) at about 45° C. followed by one or more washes in 0.2×SSC/0.1% SDS at about 50-65° C.) under highly stringent conditions (e.g., hybridization to filter-bound nucleic acid in 6×SSC at about 45° C. followed by one or more washes in 0.1×SSC/0.2% SDS at about 68° C.), or under other stringent hybridization conditions which are known to those of skill in the art (see, for example, Ausubel, F. M. et al., eds., 1989, Current Protocols in Molecular Biology, Vol. I, Green Publishing Associates, Inc. and John Wiley & Sons, Inc., New York at pages 6.3.1-6.3.6 and 2.10.3).

In another embodiment, an antibody that binds a C16orf54 epitope comprises an amino acid sequence of a VH CDR or an amino acid sequence of a VL CDR encoded by a nucleotide sequence that hybridizes to the complement of a nucleotide sequence encoding any one of the VH CDRs and/or VL CDRs depicted in Tables 1-29 under stringent conditions (e.g., hybridization to filter-bound DNA in 6×SSC at about 45° C. followed by one or more washes in 0.2×SSC/0.1% SDS at about 50-65° C.), under highly stringent conditions (e.g., hybridization to filter-bound nucleic acid in 6×SSC at about 45° C. followed by one or more washes in 0.1×SSC/0.2% SDS at about 68° C.), or under other stringent hybridization conditions which are known to those of skill in the art (see, for example, Ausubel, F. M. et al., eds., 1989, Current Protocols in Molecular Biology, Vol. I, Green Publishing Associates, Inc. and John Wiley & Sons, Inc., New York at pages 6.3.1-6.3.6 and 2.10.3)

In a further embodiment, monoclonal antibodies or antibody fragments can be isolated from antibody phage libraries generated using the techniques described in, e.g., Antibody Phage Display: Methods and Protocols, P. M. O'Brien and R. Aitken, eds, Humana Press, Totawa N.J., 2002. In principle, synthetic antibody clones are selected by screening phage libraries containing phage that display various fragments of antibody variable region (Fv) fused to phage coat protein. Such phage libraries are screened for against the desired antigen. Clones expressing Fv fragments capable of binding to the desired antigen are adsorbed to the antigen and thus separated from the non-binding clones in the library. The binding clones are then eluted from the antigen, and can be further enriched by additional cycles of antigen adsorption/elution.

Variable domains can be displayed functionally on phage, either as single-chain Fv (scFv) fragments, in which VH and VL are covalently linked through a short, flexible peptide, or as Fab fragments, in which they are each fused to a constant domain and interact non-covalently, as described in Winter et al., Ann. Rev. Immunol., 12: 433-455 (1994).

Repertoires of VH and VL genes can be separately cloned by polymerase chain reaction (PCR) and recombined randomly in phage libraries, which can then be searched for antigen-binding clones as described in Winter et al., supra. Libraries from immunized sources provide high-affinity antibodies to the immunogen without the requirement of constructing hybridomas. Alternatively, the naive repertoire can be cloned to provide a single source of human antibodies to a wide range of non-self and also self antigens without any immunization as described by Griffiths et al., EMBO J, 12: 725-734 (1993). Finally, naive libraries can also be made synthetically by cloning the unrearranged V-gene segments from stem cells, and using PCR primers containing random sequence to encode the highly variable CDR3 regions and to accomplish rearrangement in vitro as described by Hoogenboom and Winter, J. Mol. Biol., 227: 381-388 (1992).

Screening of the libraries can be accomplished by various techniques known in the art. For example, C16orf54 can be used to coat the wells of adsorption plates, expressed on host cells affixed to adsorption plates or used in cell sorting, or conjugated to biotin for capture with streptavidin-coated beads, or used in any other method for panning display libraries. The selection of antibodies with slow dissociation kinetics (and good binding affinities) can be promoted by use of long washes and monovalent phage display as described in Bass et al., Proteins, 8: 309-314 (1990) and in WO 92/09690, and a low coating density of antigen as described in Marks et al., Biotechnol., 10: 779-783 (1992).

Any of the anti-C16orf54 antibodies of the invention can be obtained by designing a suitable antigen screening procedure to select for the phage clone of interest followed by construction of a full length anti-C16orf54 antibody clone using the Fv sequences from the phage clone of interest and suitable constant region (Fc) sequences described in Kabat et al., Sequences of Proteins of Immunological Interest, Fifth Edition, NIH Publication 91-3242, Bethesda Md. (1991), vols. 1-3.

3. Antibody Fragments

The present invention encompasses antibody fragments. In certain circumstances there are advantages of using antibody fragments, rather than whole antibodies. The smaller size of the fragments allows for rapid clearance, and may lead to improved access to solid tumors. For a review of certain antibody fragments, see Hudson et al. (2003) Nat. Med. 9:129-134.

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. Fab, Fv and ScFv antibody fragments can all be expressed in and secreted from E. coli or yeast cells, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Fab and F(ab′)₂ fragment with increased in vivo half-life comprising salvage receptor binding epitope residues are described in U.S. Pat. No. 5,869,046. Other techniques for the production of antibody fragments will be apparent to the skilled practitioner. In certain embodiments, an antibody is a single chain Fv fragment (scFv). See WO 93/16185; U.S. Pat. Nos. 5,571,894; and 5,587,458. Fv and scFv are the only species with intact combining sites that are devoid of constant regions; thus, they may be suitable for reduced nonspecific binding during in vivo use. scFv fusion proteins may be constructed to yield fusion of an effector protein at either the amino or the carboxy terminus of an scFv. See Antibody Engineering, ed. Borrebaeck, supra. The antibody fragment may also be a “linear antibody”, e.g., as described for example, in the references cited before. Such linear antibodies may be monospecific or multi-specific, such as bispecific.

The smallest antibody-derived binding structures are the separate variable domains (V domains) also termed single variable domain antibodies (SdAbs). Certain types of organisms, the camelids and cartilaginous fish, possess high affinity single V-like domains mounted on an Fc equivalent domain structure as part of their immune system. (Woolven et al., Immunogenetics 50: 98-101, 1999; Streltsov et al., Proc Natl Acad Sci USA. 101:12444-12449, 2004). The V-like domains (called VhH in camelids and V-NAR in sharks) typically display long surface loops, which allow penetration of cavities of target antigens. They also stabilize isolated VH domains by masking hydrophobic surface patches.

These VhH and V-NAR domains have been used to engineer sdAbs. Human V domain variants have been designed using selection from phage libraries and other approaches that have resulted in stable, high binding VL- and VH-derived domains.

Antibodies provided herein include, but are not limited to, synthetic antibodies, monoclonal antibodies, recombinantly produced antibodies, multispecific antibodies (including bi-specific antibodies), human antibodies, humanized antibodies, camelized antibodies, chimeric antibodies, intrabodies, anti-idiotypic (anti-Id) antibodies, and functional fragements of any of the above. Non-limiting examples of functional fragements include single-chain Fvs (scFv) (e.g., including monospecific, bispecific, etc.), Fab fragments, F(ab′) fragments, F(ab)2 fragements, F(ab′)2 fragments, disulfide-linked Fvs (sdFv), Fd fragments, Fv fragments, diabody, triabody, tetrabody and minibody.

In particular, antibodies provided herein include immunoglobulin molecules and immunologically active portions of immunoglobulin molecules, e.g., molecules that contain an antigen binding site that bind to a C16orf54 epitope. The immunoglobulin molecules provided herein can be of any type (e.g., IgG, IgE, IgM, IgD, IgA and IgY), class (e.g., IgG1, IgG2, IgG3, IgG4, IgA1 and IgA2) or subclass of immunoglobulin molecule.

Variants and derivatives of antibodies include antibody functional fragments that retain the ability to bind to a C16orf54 epitope. Exemplary functional fragments include Fab fragments (an antibody fragment that contains the antigen-binding domain and comprises a light chain and part of a heavy chain bridged by a disulfide bond); Fab′ (an antibody fragment containing a single anti-binding domain comprising an Fab and an additional portion of the heavy chain through the hinge region); F(ab′)2 (two Fab′ molecules joined by interchain disulfide bonds in the hinge regions of the heavy chains; the Fab′ molecules may be directed toward the same or different epitopes); a bispecific Fab (a Fab molecule having two antigen binding domains, each of which may be directed to a different epitope); a single chain Fab chain comprising a variable region, also known as, a sFv (the variable, antigen-binding determinative region of a single light and heavy chain of an antibody linked together by a chain of 10-25 amino acids); a disulfide-linked Fv, or dsFv (the variable, antigen-binding determinative region of a single light and heavy chain of an antibody linked together by a disulfide bond); a camelized VH (the variable, antigen-binding determinative region of a single heavy chain of an antibody in which some amino acids at the VH interface are those found in the heavy chain of naturally occurring camel antibodies); a bispecific sFv (a sFv or a dsFv molecule having two antigen-binding domains, each of which may be directed to a different epitope); a diabody (a dimerized sFv formed when the VH domain of a first sFv assembles with the VL domain of a second sFv and the VL domain of the first sFv assembles with the VH domain of the second sFv; the two antigen-binding regions of the diabody may be directed towards the same or different epitopes); and a triabody (a trimerized sFv, formed in a manner similar to a diabody, but in which three antigen-binding domains are created in a single complex; the three antigen binding domains may be directed towards the same or different epitopes). Derivatives of antibodies also include one or more CDR sequences of an antibody combining site. The CDR sequences may be linked together on a scaffold when two or more CDR sequences are present. In certain embodiments, the antibody comprises a single-chain Fv (“scFv”). scFvs are antibody fragments comprising the VH and VL domains of an antibody, wherein these domains are present in a single polypeptide chain. Generally, the scFv polypeptide further comprises a polypeptide linker between the VH and VL domains which enables the scFv to form the desired structure for antigen binding. For a review of scFvs see Plückthun in The Pharmacology of Monoclonal Antibodies, vol. 113, Rosenburg and Moore eds. Springer-Verlag, New York, pp. 269-315 (1994).

4. Humanized Antibodies

The invention encompasses humanized antibodies. Various methods for humanizing non-human antibodies are known in the art. For example, a humanized antibody can have one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al. (1986) Nature 321:522-525; Riechmann et al. (1988) Nature 332:323-327; Verhoeyen et al. (1988) Science 239:1534-1536), by substituting hypervariable region sequences for the corresponding sequences of a human antibody.

In some cases, the humanized antibodies are constructed by CDR grafting, in which the amino acid sequences of the six complementarity determining regions (CDRs) of the parent rodent antibody are grafted onto a human antibody framework. Padlan et al. (FASEB J. 9:133-139, 1995) determined that only about one third of the residues in the CDRs actually contact the antigen, and termed these the “specificity determining residues,” or SDRs. In the technique of SDR grafting, only the SDR residues are grafted onto the human antibody framework (Kashmiri et al., Methods 36: 25-34, 2005).

The choice of human variable domains, both light and heavy, to be used in making the humanized antibodies can be important to reduce antigenicity. According to the so-called “best-fit” method, the sequence of the variable domain of a rodent antibody is screened against the entire library of known human variable-domain sequences. The human sequence which is closest to that of the rodent is then accepted as the human framework for the humanized antibody (Sims et al. (1993) J. Immunol. 151:2296; Chothia et al. (1987) J. Mol. Biol. 196:901. Another method uses a particular framework derived from the consensus sequence of all human antibodies of a particular subgroup of light or heavy chains. The same framework may be used for several different humanized antibodies (Carter et al. (1992) Proc. Natl. Acad. Sci. USA, 89:4285; Presta et al. (1993) J. Immunol., 151:2623. In some cases, the framework is derived from the consensus sequences of the most abundant human subclasses, V_{L}6 subgroup I (V_{L}6 I) and V_{H} subgroup III (V_{H}III). In another method, human germline genes are used at the source of the framework regions.

In an alternative paradigm based on comparison of CDRs, called Superhumanization, FR homology is irrelevant. The method consists of comparison of the non-human sequence with the functional human germline gene repertoire. Those genes encoding the same or closely related canonical structures to the murine sequences are then selected. Next, within the genes sharing the canonical structures with the non-human antibody, those with highest homology within the CDRs are chosen as FR donors. Finally, the non-human CDRs are grafted onto these FRs. (Tan et al., J. Immunol. 169: 1119-1125, 2002).

It is further generally desirable that antibodies be humanized with retention of high affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. These include, for example, WAM (Whitelegg and Rees, Protein Eng. 13: 819-824, 2000), Modeller (Sali and Blundell, J. Mol. Biol. 234: 779-815, 1993), and Swiss PDB Viewer (Guex and Peitsch, Electrophoresis 18: 2714-2713, 1997). Inspection of these displays permits analysis of the likely role of the residues in the functioning of the candidate immunoglobulin sequence, e.g., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Another method for antibody humanization is based on a metric of antibody humanness termed Human String Content (HSC). This method compares the mouse sequence with the repertoire of human germline genes and the differences are scored as HSC. The target sequence is then humanized by maximizing its HSC rather than using a global identity measure to generate multiple diverse humanized variants. (Lazar et al., Mol. Immunol. 44: 1986-1998, 2007).

In contrast to the methods described above, empirical methods may be used to generate and select humanized antibodies. These methods are based upon the generation of large libraries of humanized variants and selection of the best clones using enrichment technologies or high throughput screening techniques. Antibody variants may be isolated from phage, ribosome and yeast display libraries as well as by bacterial colony screening. (Hoogenboom, Nat. Biotechnol. 23: 1105-1116, 2005; Dufner et al., Trends Biotechnol. 24: 523-529, 2006; Feldhaus et al., Nat. Biotechnol. 21: 163-70, 2003; Schlapschy et al., Protein Eng. Des. Sel. 17: 847-60, 2004).

In the FR library approach, a collection of residue variants are introduced at specific positions in the FR followed by selection of the library to select the FR that best supports the grafted CDR. The residues to be substituted may include some or all of the “Vernier” residues identified as potentially contributing to CDR structure (Foote and Winter, J. Mol. Biol. 224: 487-499, 1992), or from the more limited set of target residues identied by Baca et al. (J. Biol. Chem. 272: 10678-10684, 1997).

In FR shuffling, whole FRs are combined with the non-human CDRs instead of creating combinatorial libraries of selected residue variants. (Dall'Acqua et al., Methods 36: 43-60, 2005). The libraries may be screened for binding in a two-step selection process, first humanizing VL, followed by VH. Alternatively, a one-step FR shuffling process may be used. Such a process has been shown to be more efficient than the two-step screening, as the resulting antibodies exhibited improved biochemical and physico-chemical properties including enhanced expression, increased affinity and thermal stability (Damschroder et al., Mol. Immunol. 44: 3049-60, 2007).

The “humaneering” method is based on experimental identification of essential minimum specificity determinants (MSDs) and is based on sequential replacement of non-human fragments into libraries of human FRs and assessment of binding. It begins with regions of the CDR-3 of non-human VH and VL chains and progressively replaces other regions of the non-human antibody into the human FRs, including the CDR-1 and CDR-2 of both VH and VL. This methodology typically results in epitope retention and identification of antibodies from multiple sub-classes with distinct human V-segment CDRs. Humaneering allows for isolation of antibodies that are 91-96% homologous to human germline gene antibodies. (Alfenito, Cambridge Healthtech Institute's Third Annual PEGS, The Protein Engineering Summit, 2007).

5. Human Antibodies

Human anti-C16orf54 antibodies of the invention can be constructed by combining Fv clone variable domain sequence(s) selected from human-derived phage display libraries with known human constant domain sequences(s) as described above. Alternatively, human monoclonal anti-C16orf54 antibodies of the invention can be made by the hybridoma method. Human myeloma and mouse-human heteromyeloma cell lines for the production of human monoclonal antibodies have been described, for example, by Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).

It is also possible to produce transgenic animals (e.g., mice) that are capable, upon immunization, of producing a full repertoire of human antibodies in the absence of endogenous immunoglobulin production. Transgenic mice that express human antibody repertoires have been used to generate high-affinity human sequence monoclonal antibodies against a wide variety of potential drug targets. See, e.g., Jakobovits, A., Curr. Opin. Biotechnol. 1995, 6(5):561-6; Brüggemann and Taussing, Curr. Opin. Biotechnol. 1997, 8(4):455-8; U.S. Pat. Nos. 6,075,181 and 6,150,584; and Lonberg et al., Nature Biotechnol. 23: 1117-1125, 2005).

Alternatively, the human antibody may be prepared via immortalization of human B lymphocytes producing an antibody directed against a target antigen (such B lymphocytes may be recovered from an individual or may have been immunized in vitro). See, e.g., Cole et al., Monoclonal Antibodies and Cancer Therapy, Alan R. Liss, p. 77 (1985); Boerner et al., J. Immunol., 147 (1):86-95 (1991); and U.S. Pat. No. 5,750,373.

Gene shuffling can also be used to derive human antibodies from non-human, e.g., rodent, antibodies, where the human antibody has similar affinities and specificities to the starting non-human antibody. According to this method, which is also called “epitope imprinting” or “guided selection”, either the heavy or light chain variable region of a non-human antibody fragment obtained by phage display techniques as described herein is replaced with a repertoire of human V domain genes, creating a population of non-human chain/human chain scFv or Fab chimeras. Selection with antigen results in isolation of a non-human chain/human chain chimeric scFv or Fab wherein the human chain restores the antigen binding site destroyed upon removal of the corresponding non-human chain in the primary phage display clone, i.e. the epitope guides (imprints) the choice of the human chain partner. When the process is repeated in order to replace the remaining non-human chain, a human antibody is obtained (see PCT WO 93/06213; and Osbourn et al., Methods., 36, 61-68, 2005). Unlike traditional humanization of non-human antibodies by CDR grafting, this technique provides completely human antibodies, which have no FR or CDR residues of non-human origin. Examples of guided selection to humanize mouse antibodies towards cell surface antigens include the folate-binding protein present on ovarian cancer cells (Figini et al., Cancer Res., 58, 991-996, 1998) and CD147, which is highly expressed on hepatocellular carcinoma (Bao et al., Cancer Biol. Ther., 4, 1374-1380, 2005).

A potential disadvantage of the guided selection approach is that shuffling of one antibody chain while keeping the other constant could result in epitope drift. In order to maintain the epitope recognized by the non-human antibody, CDR retention can be applied (Klimka et al., Br. J. Cancer., 83, 252-260, 2000; Beiboer et al., J. Mol. Biol., 296, 833-49, 2000) In this method, the non-human CDR-H3 is commonly retained, as this CDR is at the center of the antigen-binding site and has proven to be the most important region of the antibody for antigen recognition. In some instances, however, CDR-H3 and CDR-L3, as well as CDR-H3, CDR-L3 and CDR-L2 of the non-human antibody may be retained.

6. Bispecific Antibodies

Bispecific antibodies are monoclonal antibodies that have binding specificities for at least two different antigens. In certain embodiments, bispecific antibodies are human or humanized antibodies. In certain embodiments, one of the binding specificities is for C16orf54 and the other is for any other antigen. In some embodiments, one of the binding specificities is for C16orf54, and the other is for a surface antigen expressed on leukemia cells, including but not limited to CD5, CD11a, CD20, CD23, CD27, CD33, CD38, CD48, CD49d, CD52, CD62L, and CD100. In some embodiments, one arm of the bispecific antibody specifically binds to C16orf54 and the other arm has the binding specificity of a known antibody used to treat CLL (for example, alemtuzumab, rituximab, ofatumumab, or lumiliximab) or AML (for example, gemtuzumab). In certain embodiments, bispecific antibodies may bind to two different epitopes of C16orf54. Bispecific antibodies may also be used to localize cytotoxic agents to cells that express C16orf54. These antibodies possess a C16orf54-binding arm and an arm which binds a cytotoxic agent, such as, e.g., saporin, anti-interferon-α, vinca alkaloid, ricin A chain, methotrexate or radioactive isotope hapten. Bispecific antibodies can be prepared as full length antibodies or antibody fragments (e.g., F(ab′)₂ bispecific antibodies).

Methods for making bispecific antibodies are known in the art, such as, for example, by co-expression of two immunoglobulin heavy chain-light chain pairs, where the two heavy chains have different specificities (Milstein and Cuello, Nature, 305: 537 (1983)). For further details of generating bispecific antibodies see, for example, Bispecific Antibodies, Kontermann, ed., Springer-Verlag, Hiedelberg (2011).

7. Multivalent Antibodies

A multivalent antibody may be internalized (and/or catabolized) faster than a bivalent antibody by a cell expressing an antigen to which the antibodies bind. The antibodies of the present invention can be multivalent antibodies (which are other than of the IgM class) with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. In certain embodiments, the dimerization domain comprises (or consists of) an Fc region or a hinge region. In this scenario, the antibody will comprise an Fc region and three or more antigen binding sites amino-terminal to the Fc region. In certain embodiments, a multivalent antibody comprises (or consists of) three to about eight antigen binding sites. In one such embodiment, a multivalent antibody comprises (or consists of) four antigen binding sites. The multivalent antibody comprises at least one polypeptide chain (for example, two polypeptide chains), wherein the polypeptide chain(s) comprise two or more variable domains. For instance, the polypeptide chain(s) may comprise VD1-(X1)n-VD2-(X2)n-Fc, wherein VD1 is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein may further comprise at least two (for example, four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

8. Effector Function Engineering

It may be desirable to modify the antibody of the invention with respect to effector function, e.g., so as to enhance antigen-dependent cell-mediated cyotoxicity (ADCC) and/or complement dependent cytotoxicity (CDC) of the antibody. This may be achieved by introducing one or more amino acid substitutions in an Fc region of the antibody. See, e.g., Lazar et al., Proc. Natl. Acad. Sci. USA 2006, 103(11):4005-4010; Presta, L. G., Curr. Opin. Immunol. 2008, 20(4):460-70; and U.S. Pat. Nos. 7,183,387; 7,332,581; and 7,335,742.

Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff et al., Cancer Research 53:2560-2565 (1993). Alternatively, an antibody can be engineered which has dual Fc regions and may thereby have enhanced complement lysis and ADCC capabilities. See Stevenson et al., Anti-Cancer Drug Design 3:219-230 (1989). To increase the serum half life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

9. Alternative Binding Agents

The invention encompasses non-immunoglobulin binding agents that specifically bind to the same epitope as an anti-C16orf54 antibody disclosed herein. In some embodiments, a binding agent is identified an agent that displaces or is displaced by an anti-C16orf54 antibody of the invention in a competive binding assay. These alternative binding agents may include, for example, any of the engineered protein scaffolds known in the art. Such scaffolds include, for example, anticalins, which are based upon the lipocalin scaffold, a protein structure characterized by a rigid beta-barrel that supports four hypervariable loops which form the ligand binding site. Novel binding specificities are engineered by targeted random mutagenesis in the loop regions, in combination with functional display and guided selection (Skerra (2008) FEBS J. 275: 2677-2683). Other suitable scaffolds may include, for example, adnectins, or monobodies, based on the tenth extracellular domain of human fibronectin III (Koide and Koide (2007) Methods Mol. Biol. 352: 95-109); affibodies, based on the Z domain of staphylococcal protein A (Nygren et al. (2008) FEBS J. 275: 2668-2676)); DARPins, based on ankyrin repeat proteins (Stumpp et al. (2008) Drug. Discov. Today 13: 695-701); fynomers, based on the SH3 domain of the human Fyn protein kinase Grabulovski et al. (2007) J. Biol. Chem. 282: 3196-3204); affitins, based on Sac7d from Sulfolobus acidolarius (Krehenbrink et al. (2008) J. Mol. Biol. 383: 1058-1068); affilins, based on human y-B-crystallin (Ebersbach et al. (2007) J. Mol. Biol. 372: 172-185); avimers, based on the A domains of membrane receptor proteins (Silverman et al. (2005) Biotechnol. 23: 1556-1561); cysteine-rich knottin peptides (Kolmar (2008) FEBS J. 275: 2684-2690); and engineered Kunitz-type inhibitors (Nixon and Wood (2006) Curr. Opin. Drug. Discov. Dev. 9: 261-268) For a review, see Gebauer and Skerra (2009) Curr. Opin. Chem. Biol. 13: 245-255.

Antibody Variants

In some embodiments, amino acid sequence modification(s) of the antibodies described herein are contemplated. For example, it may be desirable to improve the binding affinity and/or other biological properties of the antibody, including but not limited to specificity, thermostability, expression level, effector functions, glycosylation, reduced immunogenicity or solubility. In addition to the anti-C16orf54 antibodies described herein, it is contemplated that anti-C16orf54 antibody variants can be prepared. Anti-C16orf54 antibody variants can be prepared by introducing appropriate nucleotide changes into the encoding DNA, and/or by synthesis of the desired antibody or polypeptide. Those skilled in the art will appreciate that amino acid changes may alter post-translational processes of the anti-C16orf54 antibody, such as changing the number or position of glycosylation sites or altering the membrane anchoring characteristics.

In some embodiments, antibodies provided herein are chemically modified, e.g., by the covalent attachment of any type of molecule to the antibody. For example, but not by way of limitation, the antibody derivatives include antibodies that have been chemically modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formulation, metabolic synthesis of tunicamycin, etc. Additionally, the antibody may contain one or more non-classical amino acids.

Variations may be a substitution, deletion or insertion of one or more codons encoding the antibody or polypeptide that results in a change in the amino acid sequence as compared with the native sequence antibody or polypeptide. Amino acid substitutions can be the result of replacing one amino acid with another amino acid having similar structural and/or chemical properties, such as the replacement of a leucine with a serine, e.g., conservative amino acid replacements. Insertions or deletions may optionally be in the range of about 1 to 5 amino acids. In certain embodiments, the substitution, deletion or insertion includes less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the original molecule. In a specific embodiment, the substitution is a conservative amino acid substitution made at one or more predicted non-essential amino acid residues. The variation allowed may be determined by systematically making insertions, deletions or substitutions of amino acids in the sequence and testing the resulting variants for activity exhibited by the full-length or mature native sequence. In some embodiments, the substitution, deletion or insertion can be at a variable amino acid residue, such as the one or more residues designate “X” as identified in Tables 1, 3 and 4.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for antibody-directed enzyme prodrug therapy) or a polypeptide which increases the serum half-life of the antibody.

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Amino acids may be grouped according to similarities in the properties of their side chains (in A. L. Lehninger, in Biochemistry, 2nd Ed., pp. 73-75, Worth Publishers, New York (1975)):

(1) non-polar: Ala (A), Val (V), Leu (L), Ile (I), Pro (P), Phe (F), Trp (W), Met (M)

(2) uncharged polar: Gly (G), Ser (S), Thr (T), Cys (C), Tyr (Y), Asn (N), Gln (Q)

(3) acidic: Asp (D), Glu (E)

(4) basic: Lys (K), Arg (R), His(H)

Alternatively, naturally occurring residues may be divided into groups based on common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class. Such substituted residues also may be introduced into the conservative substitution sites or, into the remaining (non-conserved) sites. Accordingly, in one embodiment, an antibody or fragment thereof that binds to a C16orf54 epitope comprises an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of a murine monoclonal antibody described herein. In one embodiment, an antibody or fragment thereof that binds to a C16orf54 epitope comprises an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to an amino acid sequence depicted in Tables 1-29. In yet another embodiment, an antibody or fragment thereof that binds to a C16orf54 epitope comprises a VH CDR and/or a VL CDR amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to a VH CDR amino acid sequence depicted in Tables 1-29 and/or a VL CDR amino acid sequence depicted in Tables 1-6, 10, 12-22, 24, 25 and 29. The variations can be made using methods known in the art such as oligonucleotide-mediated (site-directed) mutagenesis, alanine scanning, and PCR mutagenesis. Site-directed mutagenesis (Carter et al., Nucl. Acids Res., 13:4331 (1986); Zoller et al., Nucl. Acids Res., 10:6487 (1987)), cassette mutagenesis (Wells et al., Gene, 34:315 (1985)), restriction selection mutagenesis (Wells et al., Philos. Trans. R. Soc. London SerA, 317:415 (1986)) or other known techniques can be performed on the cloned DNA to produce the anti-C16orf54 antibody variant DNA.

Any cysteine residue not involved in maintaining the proper conformation of the anti-C16orf54 antibody also may be substituted, generally with serine, to improve the oxidative stability of the molecule and prevent aberrant crosslinking. Conversely, cysteine bond(s) may be added to the anti-C16orf54 antibody to improve its stability (particularly where the antibody is an antibody fragment such as an Fv fragment). Cysteine-engineered antibodies, which can be used to generate antibody-drug conjugates, are described, for example, in WO 2006/034488.

In an embodiment, an anti-C16orf54 antibody molecule of the invention is a “de-immunized” antibody. A “de-immunized” anti-C16orf54 antibody is an antibody derived from a humanized or chimeric anti-C16orf54 antibody, that has one or more alterations in its amino acid sequence resulting in a reduction of immunogenicity of the antibody, compared to the respective original non-de-immunized antibody. One of the procedures for generating such antibody mutants involves the identification and removal of T-cell epitopes of the antibody molecule. In a first step, the immunogenicity of the antibody molecule can be determined by several methods, e.g., by in vitro determination of T-cell epitopes or in silico prediction of such epitopes, as known in the art. Once the critical residues for T-cell epitope function have been identified, mutations can be made to remove immunogenicity and retain antibody activity. For review, see, e.g., Jones et al., Methods in Molecular Biology 525: 405-423, 2009.

In Vitro Affinity Maturation

In an embodiment, antibody variants having an improved property such as affinity, stability, or expression level as compared to a parent antibody is in vitro affinity maturation. Like the natural prototype, in vitro affinity maturation is based on the principles of mutation and selection. Libraries of antibodies are displayed as Fab, scFv or V domain fragments either on the surface of an organism (e.g., phage, bacteria, yeast or mammalian cell) or in association (covalently or non-covalently) with their encoding mRNA or DNA. Affinity selection of the displayed antibodies allows isolation of organisms or complexes carrying the genetic information encoding the antibodies. Two or three rounds of mutation and selection using display methods such as phage display usually results in antibody fragments with affinities in the low nanomolar range. Preferred affinity matured antibodies will have nanomolar or even picomolar affinities for the target antigen.

Phage display is the most widepread method for display and selection of antibodies. The antibodies are displayed on the surface of Fd or M13 bacteriophages as fusions to the bacteriophage coat protein. Selection involves exposure to antigen to allow phage-displayed antibodies to bind their targets, a process referred to as “panning.” Phage bound to antigen are recovered and infected in bacteria to produce phage for further rounds of selection. For review, see Hoogenboom, Methods. Mol. Biol. 178: 1-37, 2002; Bradbury and Marks, J. Immuno. Methods 290: 29-49, 2004).

In the yeast display system (Boder et al., Nat. Biotech. 15: 553-57, 1997; Chao et al., Nat. Protocols 1:755-768, 2006), the antibody is displayed as single-chain variable fusions (scFv) in which the heavy and light chains are connected by a flexible linker. The scFv is fused to the adhesion subunit of the yeast agglutinin protein Aga2p, which attaches to the yeast cell wall through disulfide bonds to Aga1p. Display of a protein via Aga2p projects the protein away from the cell surface, minimizing potential interactions with other molecules on the yeast cell wall. Magnetic separation and flow cytometry are used to screen the library to select for antibodies with improved affinity or stability. Binding to a soluble antigen of interest is determined by labeling of yeast with biotinylated antigen and a secondary reagent such as streptavidin conjugated to a fluorophore. Variations in surface expression of the antibody can be measured through immunofluorescence labeling of either the hemagglutinin or c-Myc epitope tag flanking the scFv. Expression has been shown to correlate with the stability of the displayed protein, and thus antibodies can be selected for improved stability as well as affinity (Shusta et al., J. Mol. Biol. 292: 949-956, 1999). An additional advantage of yeast display is that displayed proteins are folded in the endoplasmic reticulum of the eukaryotic yeast cells, taking advantage of endoplasmic reticulum chaperones and quality-control machinery. Once maturation is complete, antibody affinity can be conveniently ‘titrated’ while displayed on the surface of the yeast, eliminating the need for expression and purification of each clone. A theoretical limitation of yeast surface display is the potentially smaller functional library size than that of other display methods; however, a recent approach uses the yeast cells' mating system to create combinatorial diversity estimated to be 10¹⁴ in size (US Patent Publication 2003/0186,374; Blaise et al., Gene 342: 211-218, 2004).

In ribosome display, antibody-ribosome-mRNA (ARM) complexes are generated for selection in a cell-free system. The DNA library coding for a particular library of antibodies is genetically fused to a spacer sequence lacking a stop codon. This spacer sequence, when translated, is still attached to the peptidyl tRNA and occupies the ribosomal tunnel, and thus allows the protein of interest to protrude out of the ribosome and fold. The resulting complex of mRNA, ribosome, and protein can bind to surface-bound ligand, allowing simultaneous isolation of the antibody and its encoding mRNA through affinity capture with the ligand. The ribosome-bound mRNA is then reversed transcribed back into cDNA, which can then undergo mutagenesis and be used in the next round of selection. (Fukuda et al., Nucleic Acids Res. 34, e127, 2006). In mRNA display, a covalent bond between antibody and mRNA is established using puromycin as an adaptor molecule (Wilson et al., Proc. Natl. Acad. Sci. USA 98, 3750-3755, 2001).

As these methods are performed entirely in vitro, they provide two main advantages over other selection technologies. First, the diversity of the library is not limited by the transformation efficiency of bacterial cells, but only by the number of ribosomes and different mRNA molecules present in the test tube. Second, random mutations can be introduced easily after each selection round, for example, by non-proofreading polymerases, as no library must be transformed after any diversification step.

Diversity may be introduced into the CDRs or the whole V genes of the antibody libraries in a targeted manner or via random introduction. The former approach includes sequentially targeting all the CDRs of an antibody via a high or low level of mutagenesis or targeting isolated hot spots of somatic hypermutations (Ho, et al., J. Biol. Chem. 280: 607-617, 2005) or residues suspected of affecting affinity on experimental basis or structural reasons. Random mutations can be introduced throughout the whole V gene using E. coli mutator strains, error-prone replication with DNA polymerases (Hawkins et al., J. Mol. Biol. 226: 889-896, 1992) or RNA replicases. Diversity may also be introduced by replacement of regions that are naturally diverse via DNA shuffling or similar techniques ((Lu et al., J. Biol. Chem. 278: 43496-43507, 2003; U.S. Pat. No. 5,565,332; U.S. Pat. No. 6,989,250). Alternative techniques target hypervariable loops extending into framework-region residues (Bond et al., J. Mol. Biol. 348: 699-709, 2005) employ loop deletions and insertions in CDRs or use hybridization-based diversification (US Patent Publication No. 2004/0005709). Additional methods of generating diversity in CDRs are disclosed in U.S. Pat. No. 7,985,840.

Screening of the libraries can be accomplished by various techniques known in the art. For example, C16orf54 can be immobilized onto solid supports, columns, pins or cellulose/poly(vinylidene fluoride) membranes/other filters, expressed on host cells affixed to adsorption plates or used in cell sorting, or conjugated to biotin for capture with streptavidin-coated beads, or used in any other method for panning display libraries.

For review of in vitro affinity maturation methods, see Hoogenboom, Nature Biotechnology 23: 1105-1116, 2005 and Quiroz and Sinclair, Revista Ingeneria Biomedia 4: 39-51, 2010 and references therein.

Modifications of Anti-C16orf54 Antibodies

Covalent modifications of anti-C16orf54 antibodies are included within the scope of this invention. Covalent modifications include reacting targeted amino acid residues of an anti-C16orf54 antibody with an organic derivatizing agent that is capable of reacting with selected side chains or the N- or C-terminal residues of the anti-C16orf54 antibody. Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains (T.E. Creighton, Proteins: Structure and Molecular Properties, W.H. Freeman & Co., San Francisco, pp. 79-86 (1983)), acetylation of the N-terminal amine, and amidation of any C-terminal carboxyl group.

Other types of covalent modification of the anti-C16orf54 antibody included within the scope of this invention include altering the native glycosylation pattern of the antibody or polypeptide (Beck et al., Curr. Pharm. Biotechnol. 9: 482-501, 2008; Walsh, Drug Discov. Today 15: 773-780, 2010), and linking the antibody to one of a variety of nonproteinaceous polymers, e.g., polyethylene glycol (PEG), polypropylene glycol, or polyoxyalkylenes, in the manner set forth in U.S. Pat. Nos. 4,640,835; 4,496,689; 4,301,144; 4,670,417; 4,791,192 or 4,179,337.

The anti-C16orf54 antibody of the present invention may also be modified to form chimeric molecules comprising an anti-C16orf54 antibody fused to another, heterologous polypeptide or amino acid sequence, e.g., an epitope tag (Terpe, Appl. Microbiol. Biotechnol. 60: 523-533, 2003) or the Fc region of an IgG molecule (Aruffo, “Immunoglobulin fusion proteins” in Antibody Fusion Proteins, S. M. Chamow and A. Ashkenazi, eds., Wiley-Liss, New York, 1999, pp. 221-242).

Also provided herein are fusion proteins comprising an antibody provided herein that binds to a C16orf54 antigen and a heterologous polypeptide. In some embodiments, the heterologous polypeptide to which the antibody is fused is useful for targeting the antibody to cells having cell surface-expressed C16orf54.

Also provided herein are panels of antibodies that bind to a C16orf54 antigen. In specific embodiments, panels of antibodies have different association rate constants different dissociation rate constants, different affinities for C16orf54 antigen, and/or different specificities for a C16orf54 antigen. In some embodiments, the panels comprise or consist of about 10, about 25, about 50, about 75, about 100, about 125, about 150, about 175, about 200, about 250, about 300, about 350, about 400, about 450, about 500, about 550, about 600, about 650, about 700, about 750, about 800, about 850, about 900, about 950, or about 1000 antibodies or more. Panels of antibodies can be used, for example, in 96 well or 384 well plates, such as for assays such as ELISAs.

Preparation of Anti-C16orf54 Antibodies

Anti-C16orf54 antibodies may be produced by culturing cells transformed or transfected with a vector containing anti-C16orf54 antibody-encoding nucleic acid. Polynucleotide sequences encoding polypeptide components of the antibody of the invention can be obtained using standard recombinant techniques. Desired polynucleotide sequences may be isolated and sequenced from antibody producing cells such as ‘ybridomas cells. Alternatively, polynucleotides can be synthesized using nucleotide synthesizer or PCR techniques. Once obtained, sequences encoding the polypeptides are inserted into a recombinant vector capable of replicating and expressing heterologous polynucleotides in host cells. Many vectors that are available and known in the art can be used for the purpose of the present invention. Selection of an appropriate vector will depend mainly on the size of the nucleic acids to be inserted into the vector and the particular host cell to be transformed with the vector. Host cells suitable for expressing antibodies of the invention include prokaryotes such as Archaebacteria and Eubacteria, including Gram-negative or Gram-positive organisms, eukaryotic microbes such as filamentous fungi or yeast, invertebrate cells such as insect or plant cells, and vertebrate cells such as mammalian host cell lines. Host cells are transformed with the above-described expression vectors and cultured in conventional nutrient media modified as appropriate for inducing promoters, selecting transformants, or amplifying the genes encoding the desired sequences. Antibodies produced by the host cells are purified using standard protein purification methods as known in the art.

Methods for antibody production including vector construction, expression and purification are further described in Plückthun et al., (1996) in Antibody Engineering: Producing antibodies in Escherichia coli: From PCR to fermentation (McCafferty, J., Hoogenboom, H. R., and Chiswell, D. J., eds), 1 Ed., pp. 203-252, IRL Press, Oxford; Kwong, K. & Rader, C. E. coli expression and purification of Fab antibody fragments. Current protocols in protein science editorial board John E Coligan et al., Chapter 6, Unit 6.10 (2009); Tachibana and Takekoshi, “Production of Antibody Fab Fragments in Escherischia coli,” in Antibody Expression and Production, M. Al-Rubeai, Ed., Springer, New York, 2011; Therapeutic Monoclonal Antibodies: From Bench to Clinic (ed Z. An), John Wiley & Sons, Inc., Hoboken, N.J., USA.

It is, of course, contemplated that alternative methods, which are well known in the art, may be employed to prepare anti-C16orf54 antibodies. For instance, the appropriate amino acid sequence, or portions thereof, may be produced by direct peptide synthesis using solid-phase techniques (see, e.g., Stewart et al., Solid-Phase Peptide Synthesis, W.H. Freeman Co., San Francisco, Calif. (1969); Merrifield, J. Am. Chem. Soc., 85:2149-2154 (1963)). In vitro protein synthesis may be performed using manual techniques or by automation. Various portions of the anti-C16orf548 antibody may be chemically synthesized separately and combined using chemical or enzymatic methods to produce the desired anti-C16orf54 antibody. Alternatively, antibodies may be purified from cells or bodily fluids, such as milk, of a transgenic animal engineered to express the antibody, as disclosed, for example, in U.S. Pat. No. 5,545,807 and U.S. Pat. No. 5,827,690.

Immunoconiuqates

The invention also provides immunoconjugates (interchangably referred to as “antibody drug conjugates,” or “ADCs”) comprising any one of the anti-C16orf54 antibodies of the invention covalently bound by a synthetic linker to one or more cytotoxic agents. ADCs combine the high specificity of monoclonal antibodies with the pharmacological potency of cytotoxic molecules, allowing specific targeting of cytotoxic agents to tumor cells and avoiding the nonspecific toxicity of most anticancer drugs. For review, see, e.g., Carter and Senter, Cancer J. 14: 154-169 (2008); Ducry and Stump, Bioconjugate Chem. 21:5-13 (2010); Beck et al., Discov. Med. 10: 329-339 (2010).

Cytotoxic agents for use in the immunoconjugates of the invention may include chemotherapeutic agents, drugs or growth inhibitory agents as described above, toxins (e.g., an enzymatically active toxin of bacterial, fungal, plant or animal origin, or fragments thereof) or radioisotopes. In some embodiments, the immunoconjugate comprises a DNA binder (e.g., calicheamycin) or a tubulin depolymerization agent (e.g., a maytansinoid or an auristatin). The present invention further contemplates an immunoconjugate formed between an antibody and a compound with nucleolytic activity (e.g., a ribonuclease or a DNA endonuclease such as a deoxyribonuclease; DNase).

Enzymatically active toxins and fragments thereof that can be used in the immunoconjugates of the invention include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. See, e.g., WO 93/21232.

A variety of radioactive isotopes are available for the production of radioconjugated antibodies. Examples include At²¹¹, I4, I4, Y4, Re4, Re4, Sm4, Bi4, P4, Pb4 and radioactive isotopes of Lu. When the conjugate is used for detection, it may comprise a radioactive atom for scintigraphic studies, for example tc4 or I4, or a spin label for nuclear magnetic resonance (NMR) imaging (also known as magnetic resonance imaging, MRI), such as iodine-123 again, iodine-131, indium-111, fluorine-19, carbon-13, nitrogen-15, oxygen-17, gadolinium, manganese or iron. The radioisotopes may be incorporated in the conjugate in known ways as described, e.g., in Reilly, “The radiochemistry of monoclonal antibodies and peptides,” in Monoclonal Antibody and Peptide-Targeted Radiotherapy of Cancer, R. M. Reilly, ed., Wiley, Hoboken N.J., 2010.

In some embodiments, antibodies provided herein are conjugated or recombinantly fused to a diagnostic, detectable or therapeutic agent or any other molecule. The conjugated or recombinantly fused antibodies can be useful, e.g., for monitoring or prognosing the onset, development, progression and/or severity of a C16orf54-mediated disease as part of a clinical testing procedure, such as determining the efficacy of a particular therapy.

Such diagnosis and detection can accomplished, for example, by coupling the antibody to detectable substances including, but not limited to, various enzymes, such as, but not limited to, horseradish peroxidase, alkaline phosphatase, beta-galactosidase, or acetylcholinesterase; prosthetic groups, such as, but not limited to, streptavidin/biotin and avidin/biotin; fluorescent materials, such as, but not limited to, umbelliferone, fluorescein, fluorescein isothiocynate, rhodamine, dichlorotriazinylamine fluorescein, dansyl chloride or phycoerythrin; luminescent materials, such as, but not limited to, luminol; bioluminescent materials, such as but not limited to, luciferase, luciferin, and aequorin; chemiluminescent material, such as but not limited to, an acridinium based compound or a HALOTAG; radioactive materials, such as, but not limited to, iodine (¹³¹I, ¹²⁵I, ¹²³I, and ¹²¹I), carbon (¹⁴C), sulfur (³⁵S), tritium (³H), indium (¹¹⁵In, ¹¹³In, ¹¹²In, and ¹¹¹In,), technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶HO, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, ¹¹³Sn, and ¹¹⁷Sn; and positron emitting metals using various positron emission tomographies, and non-radioactive paramagnetic metal ions.

Also provided herein are antibodies that are conjugated or recombinantly fused to a therapeutic moiety (or one or more therapeutic moieties), as well as uses thereof. The antibody may be conjugated or recombinantly fused to a therapeutic moiety, such as a cytotoxin, e.g., a cytostatic or cytocidal agent, a therapeutic agent or a radioactive metal ion, e.g., alpha-emitters. A cytotoxin or cytotoxic agent includes any agent that is detrimental to cells. Therapeutic moieties include, but are not limited to, antimetabolites (e.g., methotrexate, 6-mercaptopurine, 6-thioguanine, cytarabine, 5-fluorouracil decarbazine); alkylating agents (e.g., mechlorethamine, thioepa chlorambucil, melphalan, carmustine (BCNU) and lomustine (CCNU), cyclothosphamide, busulfan, dibromomannitol, streptozotocin, mitomycin C, and cisdichlorodiamine platinum (II) (DDP), and cisplatin); anthracyclines (e.g., daunorubicin (formerly daunomycin) and doxorubicin); antibiotics (e.g., d actinomycin (formerly actinomycin), bleomycin, mithramycin, and anthramycin (AMC)); Auristatin molecules (e.g., auristatin PHE, auristatin F, monomethyl auristatin E, bryostatin 1, and solastatin 10; see Woyke et al., Antimicrob. Agents Chemother. 46:3802-8 (2002), Woyke et al., Antimicrob. Agents Chemother. 45:3580-4 (2001), Mohammad et al., Anticancer Drugs 12:735-40 (2001), Wall et al., Biochem. Biophys. Res. Commun. 266:76-80 (1999), Mohammad et al., Int. J. Oncol. 15:367-72 (1999), all of which are incorporated herein by reference); hormones (e.g., glucocorticoids, progestins, androgens, and estrogens), DNA-repair enzyme inhibitors (e.g., etoposide or topotecan), kinase inhibitors (e.g., compound ST1571, imatinib mesylate (Kantarjian et al., Clin Cancer Res. 8(7):2167-76 (2002)); cytotoxic agents (e.g., paclitaxel, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D,1-dehydrotestosterone, glucorticoids, procaine, tetracaine, lidocaine, propranolol, and puromycin and analogs or homologs thereof and those compounds disclosed in U.S. Pat. Nos. 6,245,759, 6,399,633, 6,383,790, 6,335,156, 6,271,242, 6,242,196, 6,218,410, 6,218,372, 6,057,300, 6,034,053, 5,985,877, 5,958,769, 5,925,376, 5,922,844, 5,911,995, 5,872,223, 5,863,904, 5,840,745, 5,728,868, 5,648,239, 5,587,459); farnesyl transferase inhibitors (e.g., R115777, BMS-214662, and those disclosed by, for example, U.S. Pat. Nos. 6,458,935, 6,451,812, 6,440,974, 6,436,960, 6,432,959, 6,420,387, 6,414,145, 6,410,541, 6,410,539, 6,403,581, 6,399,615, 6,387,905, 6,372,747, 6,369,034, 6,362,188, 6,342,765, 6,342,487, 6,300,501, 6,268,363, 6,265,422, 6,248,756, 6,239,140, 6,232,338, 6,228,865, 6,228,856, 6,225,322, 6,218,406, 6,211,193, 6,187,786, 6,169,096, 6,159,984, 6,143,766, 6,133,303, 6,127,366, 6,124,465, 6,124,295, 6,103,723, 6,093,737, 6,090,948, 6,080,870, 6,077,853, 6,071,935, 6,066,738, 6,063,930, 6,054,466, 6,051,582, 6,051,574, and 6,040,305); topoisomerase inhibitors (e.g., camptothecin; irinotecan; SN-38; topotecan; 9-aminocamptothecin; GG-211 (GI 147211); DX-8951f; IST-622; rubitecan; pyrazoloacridine; XR-5000; saintopin; UCE6; UCE1022; TAN-1518A; TAN 1518B; KT6006; KT6528; ED-110; NB-506; ED-110; NB-506; and rebeccamycin); bulgarein; DNA minor groove binders such as Hoescht dye 33342 and Hoechst dye 33258; nitidine; fagaronine; epiberberine; coralyne; beta-lapachone; BC-4-1; bisphosphonates (e.g., alendronate, cimadronte, clodronate, tiludronate, etidronate, ibandronate, neridronate, olpandronate, risedronate, piridronate, pamidronate, zolendronate) HMG-CoA reductase inhibitors, (e.g., lovastatin, simvastatin, atorvastatin, pravastatin, fluvastatin, statin, cerivastatin, lescol, lupitor, rosuvastatin and atorvastatin); antisense oligonucleotides (e.g., those disclosed in the U.S. Pat. Nos. 6,277,832, 5,998,596, 5,885,834, 5,734,033, and 5,618,709); adenosine deaminase inhibitors (e.g., Fludarabine phosphate and 2-Chlorodeoxyadenosine); ibritumomab tiuxetan (Zevalin®); tositumomab (Bexxar®)) and pharmaceutically acceptable salts, solvates, clathrates, and prodrugs thereof.

Further, an antibody provided herein may be conjugated or recombinantly fused to a therapeutic moiety or drug moiety that modifies a given biological response. Therapeutic moieties or drug moieties are not to be construed as limited to classical chemical therapeutic agents. For example, the drug moiety may be a protein, peptide, or polypeptide possessing a desired biological activity. Such proteins may include, for example, a toxin such as abrin, ricin A, pseudomonas exotoxin, cholera toxin, or diphtheria toxin; a protein such as tumor necrosis factor, γ-interferon, α-interferon, nerve growth factor, platelet derived growth factor, tissue plasminogen activator, an apoptotic agent, e.g., TNF-γ, TNF-γ, AIM I (see, International Publication No. WO 97/33899), AIM II (see, International Publication No. WO 97/34911), Fas Ligand (Takahashi et al., 1994, J. Immunol., 6:1567-1574), and VEGF (see, International Publication No. WO 99/23105), an anti-angiogenic agent, e.g., angiostatin, endostatin or a component of the coagulation pathway (e.g., tissue factor); or, a biological response modifier such as, for example, a lymphokine (e.g., interferon gamma, interleukin-1 (“IL-1”), interleukin-2 (“IL-2”), interleukin-5 (“IL-5”), interleukin-6 (“IL-6”), interleukin-7 (“IL-7”), interleukin 9 (“IL-9”), interleukin-10 (“IL-10”), interleukin-12 (“IL-12”), interleukin-15 (“IL-15”), interleukin-23 (“IL-23”), granulocyte macrophage colony stimulating factor (“GM-CSF”), and granulocyte colony stimulating factor (“G-CSF”)), or a growth factor (e.g., growth hormone (“GH”)), or a coagulation agent (e.g., calcium, vitamin K, tissue factors, such as but not limited to, Hageman factor (factor XII), high-molecular-weight kininogen (HMWK), prekallikrein (PK), coagulation proteins-factors II (prothrombin), factor V, XIIa, VIII, XIIIa, XI, XIa, IX, IXa, X, phospholipid, and fibrin monomer).

Also provided herein are antibodies that are recombinantly fused or chemically conjugated (covalent or non-covalent conjugations) to a heterologous protein or polypeptide (or fragment thereof, for example, to a polypeptide of about 10, about 20, about 30, about 40, about 50, about 60, about 70, about 80, about 90 or about 100 amino acids) to generate fusion proteins, as well as uses thereof. In particular, provided herein are fusion proteins comprising an antigen-binding fragment of an antibody provided herein (e.g., a Fab fragment, Fd fragment, Fv fragment, F(ab)₂ fragment, a VH domain, a VH CDR, a VL domain or a VL CDR) and a heterologous protein, polypeptide, or peptide. In one embodiment, the heterologous protein, polypeptide, or peptide that the antibody is fused to is useful for targeting the antibody to a particular cell type, such as a cell that expresses C16orf54 or an C16orf54 receptor. For example, an antibody that binds to a cell surface receptor expressed by a particular cell type (e.g., an immune cell) may be fused or conjugated to a modified antibody provided herein.

In addition, an antibody provided herein can be conjugated to therapeutic moieties such as a radioactive metal ion, such as alpha-emitters such as ²¹³Bi or macrocyclic chelators useful for conjugating radiometal ions, including but not limited to, ¹³¹In, ¹³¹LU, ¹³¹Y, ¹³¹Ho, ¹³¹Sm, to polypeptides. In certain embodiments, the macrocyclic chelator is 1,4,7,10-tetraazacyclododecane-N,N′,N″,N′″-tetraacetic acid (DOTA) which can be attached to the antibody via a linker molecule. Such linker molecules are commonly known in the art and described in Denardo et al., 1998, Clin Cancer Res. 4(10):2483-90; Peterson et al., 1999, Bioconjug. Chem. 10(4):553-7; and Zimmerman et al., 1999, Nucl. Med. Biol. 26(8):943-50, each incorporated by reference in their entireties.

Moreover, antibodies provided herein can be fused to marker sequences, such as a peptide to facilitate purification. In specific embodiments, the marker amino acid sequence is a hexa-histidine peptide, such as the tag provided in a pQE vector (QIAGEN, Inc.), among others, many of which are commercially available. As described in Gentz et al., 1989, Proc. Natl. Acad. Sci. USA 86:821-824, for instance, hexa-histidine provides for convenient purification of the fusion protein. Other peptide tags useful for purification include, but are not limited to, the hemagglutinin (“HA”) tag, which corresponds to an epitope derived from the influenza hemagglutinin protein (Wilson et al., 1984, Cell 37:767), and the “FLAG” tag.

Methods for fusing or conjugating therapeutic moieties (including polypeptides) to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies 84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), Thorpe et al., 1982, Immunol. Rev. 62:119-58; U.S. Pat. Nos. 5,336,603, 5,622,929, 5,359,046, 5,349,053, 5,447,851, 5,723,125, 5,783,181, 5,908,626, 5,844,095, and 5,112,946; EP 307,434; EP 367,166; EP 394,827; PCT publications WO 91/06570, WO 96/04388, WO 96/22024, WO 97/34631, and WO 99/04813; Ashkenazi et al., Proc. Natl. Acad. Sci. USA, 88: 10535-10539, 1991; Traunecker et al., Nature, 331:84-86, 1988; Zheng et al., J. Immunol., 154:5590-5600, 1995; Vil et al., Proc. Natl. Acad. Sci. USA, 89:11337-11341, 1992, which are incorporated herein by reference in their entireties.

Fusion proteins may be generated, for example, through the techniques of gene-shuffling, motif-shuffling, exon-shuffling, and/or codon-shuffling (collectively referred to as “DNA shuffling”). DNA shuffling may be employed to alter the activities of antibodies provided herein (e.g., antibodies with higher affinities and lower dissociation rates). See, generally, U.S. Pat. Nos. 5,605,793, 5,811,238, 5,830,721, 5,834,252, and 5,837,458; Patten et al., 1997, Curr. Opinion Biotechnol.

8:724-33; Harayama, 1998, Trends Biotechnol. 16(2):76-82; Hansson et al., 1999, J. Mol. Biol. 287:265-76; and Lorenzo and Blasco, 1998, Biotechniques 24(2):308-313 (each of these patents and publications are hereby incorporated by reference in its entirety). Antibodies, or the encoded antibodies, may be altered by being subjected to random mutagenesis by error-prone PCR, random nucleotide insertion or other methods prior to recombination. A polynucleotide encoding an antibody provided herein may be recombined with one or more components, motifs, sections, parts, domains, fragments, etc. of one or more heterologous molecules.

An antibody provided herein can also be conjugated to a second antibody to form an antibody heteroconjugate as described in U.S. Pat. No. 4,676,980, which is incorporated herein by reference in its entirety.

The therapeutic moiety or drug conjugated or recombinantly fused to an antibody provided herein that binds to a C16orf54 antigen should be chosen to achieve the desired prophylactic or therapeutic effect(s). In certain embodiments, the antibody is a modified antibody. A clinician or other medical personnel should consider the following when deciding on which therapeutic moiety or drug to conjugate or recombinantly fuse to an antibody provided herein: the nature of the disease, the severity of the disease, and the condition of the subject.

Antibodies provided herein may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

The linker may be a “cleavable linker” facilitating release of the cytotoxic drug in the cell, but non-cleavable linkers are also contemplated herein. Linkers for use in the immunoconjugates of the invention include without limitation acid labile linkers (e.g., hydrazone linkers), disulfide-containing linkers, peptidase-sensitive linkers (e.g., peptide linkers comprising amino acids, for example, valine and/or citrulline such as citrulline-valine or phenylalanine-lysine), photolabile linkers, dimethyl linkers (Chari et al., Cancer Research 52:127-131 (1992); U.S. Pat. No. 5,208,020), thioether linkers, or hydrophilic linkers designed to evade multidrug transporter-mediated resistance (Kovtun et al., Cancer Res. 70: 2528-2537, 2010).

Conjugates of the antibody and cytotoxic agent may be made using a variety of bifunctional protein coupling agents such as BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate)). For example, a ricin immunotoxin can be prepared as described in Vitetta et al., Science 238:1098 (1987). The invention further contemplates that conjugates of antibodies and cytotoxic agents may be prepared using any suitable methods as disclosed in the art, e.g., in Bioconjugate Techniques, 2nd Ed., G. T. Hermanson, ed., Elsevier, San Francisco, 2008.

Conventional antibody-drug conjugation strategies have been based on random conjugation chemistries involving the c-amino group of Lys residues or the thiol group of Cys residues, which results in heterogenous conjugates. Recently developed techniques allow site-specific conjugation to antibodies, resulting in homogeneous drug loading and avoiding ADC subpopulations with altered antigen-binding or pharmacokinetics. These include engineering of “thiomabs” comprising cysteine substitutions at positions on the heavy and light chains that provide reactive thiol groups and do not disrupt immunoglobulin folding and assembly or alter antigen binding (Junutula et al., J. Immunol. Meth. 332: 41-52 (2008); Junutula et al., Nat. Biotechnol. 26: 925-932, 2008). In another method, selenocysteine is cotranslationally inserted into an antibody sequence by recoding the stop codon UGA from termination to selenocysteine insertion, allowing site specific covalent conjugation at the nucleophilic selenol group of selenocysteine in the presence of the other natural amino acids (Hofer et al., Proc. Natl. Acad. Sci. USA 105: 12451-12456 (2008); Hofer et al., Biochemistry 48(50): 12047-12057, 2009).

Antibody-drug conjugates are provided herein, including an antibody-drug conjugate of the following formulas (Ia) and (Ib):

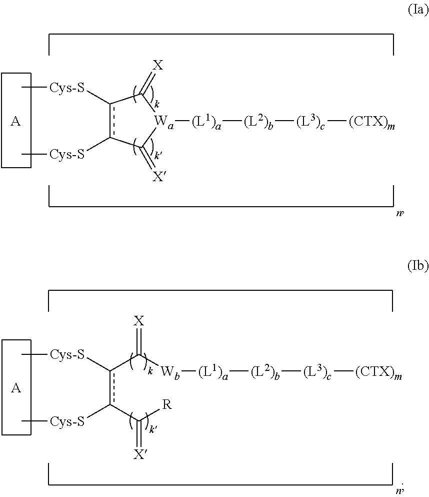

or a pharmaceutically acceptable salt thereof;

wherein:

A is an antibody or antibody fragment;

the two depicted cysteine residues are from an opened cysteine-cysteine disulfide bond in A;

each X and X′ is independently O, S, NH, or NR¹ wherein R¹ is C₁₋₆ alkyl;

Wₐ is ═N—, ═CH—, ═CHCH₂—, ═C(R²)—, or ═CHCH(R²)—; W_{b} is —NH—, —N(R¹)—, —CH₂—, —CH₂—NH—, —CH₂—N(R¹)—, —CH₂CH₂—, —CH(R²)—, or —CH₂CH(R²)—; wherein R¹ and R² are independently C₁₋₆ alkyl;

CTX is a cytotoxic agent;

R is any chemical group; or R is absent;

each L¹, L² and L³ is independently a linker selected from the group consisting of —O—, —C(O)—, —S—, —S(O)—, —S(O)₂—, —NH—, —NCH₃—, —(CH₂)_{q}—, —NH(CH₂)₂NH—, —OC(O)—, —CO₂—, —NHCH₂CH₂C(O)—, —C(O)NHCH₂CH₂NH—, —NHCH₂C(O)—, —NHC(O)—, —C(O)NH—, —NCH₃C(O)—, —C(O)NCH₃—, —(CH₂CH₂O)ₚ, —(CH₂CH₂O)ₚCH₂CH₂—, —CH₂CH₂—(CH₂CH₂O)ₚ—, —OCH(CH₂O—)₂, -(AA)ᵣ-, cyclopentanyl, cyclohexanyl, unsubstituted phenylenyl, and phenylenyl substituted by 1 or 2 substituents selected from the group consisting of halo, CF₃—, CF₃O—, CH₃O—, —C(O)OH, —C(O)OC₁₋₃ alkyl, —C(O)CH₃, —CN, —NH₂, —OH, —NHCH₃, —N(CH3)₂, and C₁₋₃ alkyl;

a, b and c are each independently an integer of 0, 1, 2 or 3, provided that at least one of a, b or c is 1;

each k and k′ is independently an integer of 0 or 1;

each p is independently an integer of 1 to 14;

each q is independently an integer from 1 to 12;

each AA is independently an amino acid;

each r is 1 to 12;

m is an integer of 1 to 4;

n is an integer of 1 to 4; and

the [symbol] bond represents a single or a double bond.

In certain embodiments of the antibody-drug conjugate of formula (Ib), R is selected from the group consisting of W, (L¹)ₐ, (L²)_{b}, (L³)_{c}, Z, W-(L¹)ₐ-(L²)_{b}-(L³)_{c}, (L¹)ₐ-(L²)_{b}-(L³)_{c}-Z, and W-(L¹)ₐ-(L²)_{b}-(L³)_{c}-Z, as defined herein. In certain embodiments, R is selected from the group consisting of W, (L¹)ₐ, (L²)_{b}, (L³)_{c}, and W-(L¹)ₐ-(L²)_{b}-(L³)_{c}. In certain embodiments, R is selected from the group consisting of Z, (L¹)ₐ-(L²)_{b}-(L³)_{c}-Z, and W-(L¹)ₐ-(L²)_{b}-(L³)_{c}-Z.

In certain embodiments of the antibody-drug conjugate of formula (Ib), R is a detectable probe. In certain embodiments, R is a fluorophore, chromophore, radiolabel, enzyme, antibody or antibody fragment. In certain embodiments, R is an antibody fragment.

In certain embodiments of the antibody-drug conjugate of formula (Ib), R is bonded to the rest of the linker molecule via an amide, an N—(C₁₋₆ alkyl)amide, a carbamate, an N—(C₁₋₆ alkyl)carbamate, an amine, an N—(C₁₋₆ alkyl)amine, an ether, a thioether, an urea, an N—(C₁₋₆ alkyl)urea, or an N,N-di(C₁₋₆ alkyl)urea bond.

In certain embodiments of the antibody-drug conjugate of formula (Ia) or (Ib), CTX is bonded to (L¹)ₐ-(L²)_{b}-(L³)_{c} via a group selected from —NHC(O)—, —NHC(O)O—, —N(C₁₋₃alkyl)C(O)O—, —NH—, —N(C₁₋₃alkyl)-, —N(C₁₋₃alkyl)C(O)NH— and —N(C₁₋₃alkyl)C(O)N(C₁₋₃alkyl)-.

In certain embodiments of the antibody-drug conjugate of formula (Ia) or (Ib), CTX is selected from a from the group consisting of a tubulin stabilizer, a tubulin destabilizer, a DNA alkylator, a DNA minor groove binder, a DNA intercalator, a topoisomerase I inhibitor, a topoisomerase II inhibitor, a gyrase inhibitor, a protein synthesis inhibitor, a proteosome inhibitor, and an anti-metabolite.

In certain embodiments of the antibody-drug conjugate of formula (Ia) or (Ib), the CTX is a chemotherapeutic agent. Those of ordinary skill in the art will be aware of appropriate chemotherapeutic agents as disclosed, for example, in Chu, E., DeVite, V. T., 2012, Physicians' Cancer Chemotherapy Drug Manual 2012 (Jones & Bartlett Learning Oncology), and similar documents.

In certain embodiments, the CTX may be any FDA-approved chemotherapeutic agent. In certain embodiments, the CTX may be any FDA-approved chemotherapeutic agent available for cancer treatment.

In certain embodiments, the CTX is selected from the group consisting of an alkylating agents, an anthracyclines, a cytoskeletal disruptors (taxanes), an epothilones, an histone deacetylase Inhibitor (HDAC), an inhibitor of Topoisomerase I, an Inhibitor of Topoisomerase II, a kinase inhibitor, a monoclonal antibodies, a nucleotide analog, a peptide antibiotic, a platinum-based agent, a retinoids, a Vinca alkaloid or a derivative thereof, and radioisotope.

In certain embodiments, the CTX is selected from the group consisting of Actinomycin, all-trans retinoic acid, Azacitidine, Azathioprine, Bleomycin, Bortezomib, Carboplatin, Capecitabine, Cisplatin, Chlorambucil, Cyclophosphamide, Cytarabine, Daunorubicin, Docetaxel, Doxifluridine, Doxorubicin, Epirubicin, Epothilone, Etoposide, Fluorouracil, Gemcitabine, Hydroxyurea, Idarubicin, Imatinib, Irinotecan, Mechlorethamine, Mercaptopurine, Methotrexate, Mitoxantrone, Oxaliplatin, Paclitaxel, Pemetrexed, Teniposide, Tioguanine, Topotecan, Valrubicin, Vinblastine, Vincristine, Vindesine, and Vinorelbine.

In certain embodiments, the CTX is selected from the group consisting of a tubulin stabilizer, a tubulin destabilizer, a DNA alkylator, a DNA minor groove binder, a DNA intercalator, a topoisomerase I inhibitor, a topoisomerase II inhibitor, a gyrase inhibitor, a protein synthesis inhibitor, a proteosome inhibitor, and an anti-metabolite.

In certain embodiments, the CTX is selected from the group consisting of Actinomycin D, Amonafide, an auristatin, benzophenone, benzothiazole, a calicheamicin, Camptothecin, CC-1065 (NSC 298223), Cemadotin, Colchicine, Combretastatin A4, Dolastatin, Doxorubicin, Elinafide, Emtansine (DM1), Etoposide, KF-12347 (Leinamycin), a maytansinoid, Methotrexate, Mitoxantrone, Nocodazole, Proteosome Inhibitor 1 (PSI 1), Roridin A, T-2 Toxin (trichothecene analog), Taxol, a tubulysin, Velcade®, and Vincristine. In certain embodiments, the CTX is an auristatin, a calicheamicin, a maytansinoid, or a tubulysin. In certain embodiments, the CTX is monomethylauristatin E, monomethylauristatin F, calicheamicin γ, mertansine, tubulysin T3, or tubulysin T4, the structures for which are provided below:

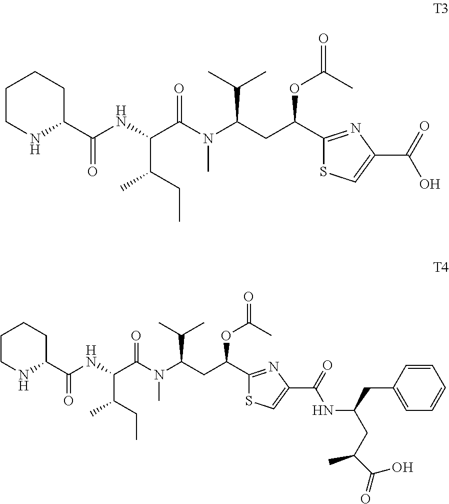 [SMILES: CC[C@H](C)[C@H](NC(=O)[C@H]1CCCCN1)C(=O)N(C)[C@H](C[C@@H](OC(C)=O)c1nc(C(=O)O)cs1)C(C)C ; CC[C@H](C)[C@H](NC(=O)[C@H]1CCCCN1)C(=O)N(C)[C@H](C[C@@H](OC(C)=O)c1nc(C(=O)N[C@@H](Cc2ccccc2)C[C@H](C)C(=O)O)cs1)C(C)C]

Pharmaceutical Formulations

The antibodies or immunoconjugates, e.g., antibody-drug conjugates (ADC), of the invention may be administered by any route appropriate to the condition to be treated. The antibody or ADC will typically be administered parenterally, e.g., infusion, subcutaneous, intramuscular, intravenous, intradermal, intrathecal and epidural.

For treating cancers, in one embodiment, the antibody or antibody-drug conjugate is administered via intravenous infusion. The dosage administered via infusion is in the range of about 1 μg/m² to about 10,000 μg/m² per dose, generally one dose per week for a total of one, two, three or four doses. Alternatively, the dosage range is of about 1 μg/m² to about 1000 μg/m², about 1 μg/m² to about 800 μg/m², about 1 μg/m² to about 600 μg/m², about 1 μg/m² to about 400 μg/m², about 10 μg/m² to about 500 μg/m², about 10 μg/m² to about 300 μg/m², about 10 μg/m² to about 200 μg/m², and about 1 μg/m² to about 200 μg/m². The dose may be administered once per day, once per week, multiple times per week, but less than once per day, multiple times per month but less than once per day, multiple times per month but less than once per week, once per month or intermittently to relieve or alleviate symptoms of the disease. Administration may continue at any of the disclosed intervals until remission of the tumor or symptoms of the cancer being treated. Administration may continue after remission or relief of symptoms is achieved where such remission or relief is prolonged by such continued administration.

In one aspect, the invention further provides pharmaceutical formulations comprising at least one anti-C16orf54 antibody of the invention and/or at least one immunoconjugate thereof and/or at least one anti-C16orf54 antibody-drug conjugate of the invention. In some embodiments, a pharmaceutical formulation comprises 1) an anti-C16orf54 antibody and/or an anti-C16orf54 antibody-drug conjugate and/or an immunoconjugate thereof, and 2) a pharmaceutically acceptable carrier. In some embodiments, a pharmaceutical formulation comprises 1) an anti-C16orf54 antibody and/or an immunoconjugate thereof, and optionally, 2) at least one additional therapeutic agent.

Pharmaceutical formulations comprising an antibody or immunoconjugate of the invention or the antibody-drug conjugate of the invention are prepared for storage by mixing the antibody or antibody-drug conjugate having the desired degree of purity with optional physiologically acceptable carriers, excipients or stabilizers (Remington's Pharmaceutical Sciences 16th edition, Osol, A. Ed. (1980)) in the form of aqueous solutions or lyophilized or other dried formulations. The formulations herein may also contain more than one active compound as necessary for the particular indication being treated, preferably those with complementary activities that do not adversely affect each other. For example, in addition to an anti-C16orf54 antibody, it may be desirable to include in the one formulation, an additional antibody, e.g., a second anti-C16orf54 antibody which binds a different epitope on the C16orf54 polypeptide, or an antibody to some other target such as a growth factor that affects the growth of the particular cancer. In some embodiments, the formulation includes an antibody to CD20 (e.g., rituximab, ofatumumab, obinutuzumab, veltuzumab, and ocrelizumab), CD23 (lumiliximab), CD52 (alemtuzumab), or CD33 (gemtuzumab). Alternatively, or additionally, the composition may further comprise a chemotherapeutic agent, cytotoxic agent, cytokine, growth inhibitory agent, anti-hormonal agent, and/or cardioprotectant. In some embodiments the formulation includes an alkylating agent (for example, chlorambucil, bendamustine hydrochloride or cyclophosphamide) a nucleoside analog (for example, fludurabine, pentostatin, cladribine or cytarabine) a corticosteroid (for example, prednisone, prednisolone or methylprednisolone), an immunomodulatory agent (for example, lenalidomide), an antibiotic (for example, doxorubicin, daunorubicin idarubicin or mitoxentrone), a synthetic flavon such as flavopiridol, a Bcl2 antagonist, such as oblimersen or ABT-263, a hypomethylating agent such as azacytidine or decitabine, an FLT3 inhibitor such as midostaurin, sorafenib and AC220. Such molecules are suitably present in combination in amounts that are effective for the purpose intended.

The antibodies or immunoconjugates of the invention may be formulated in any suitable form for delivery to a target cell/tissue, e.g., as microcapsules or macroemulsions (Remington's Pharmaceutical Sciences, 16th edition, Osol, A. Ed. (1980); Park et al., Molecules 10: 146-161 (2005); Malik et al., Curr. Drug. Deliv. 4: 141-151 (2007)); as sustained release formulations (Putney and Burke, Nature Biotechnol. 16: 153-157, (1998)) or in liposomes (Maclean et al., Int. J. Oncol. 11: 235-332 (1997); Kontermann, Curr. Opin. Mol. Ther. 8: 39-45 (2006)).

Therapeutic Methods

An antibody or immunoconjugate of the invention may be used in, for example, in vitro, ex vivo, and in vivo therapeutic methods. In one aspect, the invention provides methods for inhibiting cell growth or proliferation, either in vivo or in vitro, the method comprising exposing a cell to an anti-C16orf54 antibody or immunoconjugate thereof under conditions permissive for binding of the immunoconjugate to C16orf54. “Inhibiting cell growth or proliferation” means decreasing a cell's growth or proliferation by at least 10%, 20%, 30%, 40%, 50%, 60%, 70%, 80%, 90%, 95%, or 100%, and includes inducing cell death. In certain embodiments, the cell is a tumor cell. In certain embodiments, the cell is a leukemia cell, a lymphoma cell, a myeloma cell, a solid tumor cell such as a breast cancer cell, a pancreatic cancer cell or a metastatic cancer cell of any of the aforementioned cancer cells.

In one aspect, an antibody or immunoconjugate of the invention is used to treat or prevent a cell proliferative disorder, such as cancer. In certain embodiments, the cell proliferative disorder is associated with increased expression and/or activity of C16orf54. For example, in certain embodiments, the cell proliferative disorder is associated with increased expression of C16orf54 on the surface of a cancer cell. Examples of cell proliferative disorders to be treated by the antibodies or immunoconjugates of the invention include, but are not limited to, a leukemia such as CLL, ALL, AML or CML, a lymphoma, a myeloma, solid tumors such as breast cancer or pancreatic cancer, and metastases of any of these cancers.

In one aspect, the invention provides methods for treating a cell proliferative disorder comprising administering to an individual an effective amount of an anti-C16orf54 antibody or immunoconjugate thereof. In certain embodiments, a method for treating a cell proliferative disorder comprises administering to an individual an effective amount of a pharmaceutical formulation comprising an anti-C16orf54 antibody or anti-C16orf54 immunoconjugate and, optionally, at least one additional therapeutic agent, such as those provided below. In one embodiment, an anti-C16orf54 antibody or immunoconjugate can be used for targeting C16orf54 on cancer cells by contacting the antibody or immunoconjugate with C16orf54 to form an antibody or immunoconjugate-antigen complex such that a conjugated cytotoxic agent of the immunoconjugate accesses the interior of the cell. In one embodiment, the bound antibody or immunoconjugate is internalized into the cancer cell expressing C16orf54.

An anti-C16orf54 antibody or immunoconjugate can be administered to a human for therapeutic purposes. Moreover, an anti-C16orf54 antibody or immunoconjugate can be administered to a non-human mammal expressing C16orf54 with which the antibody cross-reacts (e.g., a primate, pig, rat, or mouse) for veterinary purposes or as an animal model of human disease. Regarding the latter, such animal models may be useful for evaluating the therapeutic efficacy of antibodies or immunoconjugates of the invention (e.g., testing of dosages and time courses of administration).

Antibodies or immunoconjugates of the invention can be used either alone or in combination with other compositions in a therapy. For instance, an antibody or immunoconjugate of the invention may be co-administered with at least one additional therapeutic agent and/or adjuvant. In certain embodiments, an additional therapeutic agent is a cytotoxic agent, a chemotherapeutic agent, or a growth inhibitory agent. In one of such embodiments, a chemotherapeutic agent is an agent or a combination of agents such as, for example, cyclophosphamide, hydroxydaunorubicin, adriamycin, doxorubincin, vincristine (Oncovin™) prednisolone, CHOP, CVP, or COP, wherein the combination therapy is useful in the treatment of cancers. In some embodiments, the additional compound is a therapeutic antibody other than an anti-C16orf54 antibody.

Such combination therapies noted above encompass combined administration (where two or more therapeutic agents are included in the same or separate formulations), and separate administration, in which case, administration of the antibody or immunoconjugate of the invention can occur prior to, simultaneously, and/or following, administration of the additional therapeutic agent and/or adjuvant. Antibodies or immunoconjugates of the invention can also be used in combination with additional therapeutic regimens including without limitation radiation therapy and/or bone marrow and peripheral blood transplants.

An antibody or immunoconjugate of the invention (and any additional therapeutic agent or adjuvant) can be administered by any suitable means, including parenteral, subcutaneous, intraperitoneal, intrapulmonary, and intranasal, and, if desired for local treatment, intralesional administration. Parenteral infusions include intramuscular, intravenous, intraarterial, intraperitoneal, or subcutaneous administration. In addition, the antibody or immunoconjugate is suitably administered by pulse infusion, particularly with declining doses of the antibody or immunoconjugate. Dosing can be by any suitable route, e.g., by injections, such as intravenous or subcutaneous injections, depending in part on whether the administration is brief or chronic.

Antibodies or immunoconjugates of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody or immunoconjugate need not be, but is optionally formulated with one or more agents currently used to prevent or treat the disorder in question. The effective amount of such other agents depends on the amount of antibody or immunoconjugate present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody or immunoconjugate of the invention (when used alone or in combination with one or more other additional therapeutic agents, such as chemotherapeutic agents) will depend on the type of disease to be treated, the type of antibody or immunoconjugate, the severity and course of the disease, whether the antibody or immunoconjugate is administered for preventive or therapeutic purposes, previous therapy, the patient's clinical history and response to the antibody or immunoconjugate, and the discretion of the attending physician. The antibody or immunoconjugate is suitably administered to the patient at one time or over a series of treatments. Depending on the type and severity of the disease, about 1 μg/kg to 100 mg/kg (e.g., 0.1 mg/kg-20 mg/kg) of antibody or immunoconjugate can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the antibody or immunoconjugate would be in the range from about 0.05 mg/kg to about 10 mg/kg. Thus, one or more doses of about 0.5 mg/kg, 2.0 mg/kg, 4.0 mg/kg or 10 mg/kg (or any combination thereof) of antibody or immunoconjugate may be administered to the patient. Such doses may be administered intermittently, e.g., every week or every three weeks (e.g., such that the patient receives from about two to about twenty, or e.g., about six doses of the antibody or immunoconjugate). An initial higher loading dose, followed by one or more lower doses may be administered. An exemplary dosing regimen comprises administering an initial loading dose of about 4 mg/kg, followed by a weekly maintenance dose of about 2 mg/kg of the antibody. However, other dosage regimens may be useful. The progress of this therapy is easily monitored by conventional techniques and assays.

Diagnostic Methods and Methods of Detection

In one aspect, anti-C16orf54 antibodies and immunoconjugates of the invention are useful for detecting the presence of C16orf54 in a biological sample. The term “detecting” as used herein encompasses quantitative or qualitative detection. In certain embodiments, a biological sample comprises a cell or tissue. In certain embodiments, such cells include normal and/or cancerous cells that express C16orf54 at higher levels relative to other cells, for example, a leukemia such as CLL, ALL, AML or CML, a lymphoma, a myeloma, or a solid tumor such as breast cancer or pancreatic cancer or metastases of any of these cancers.

In one aspect, the invention provides a method of detecting the presence of C16orf54 in a biological sample. In certain embodiments, the method comprises contacting the biological sample with an anti-C16orf54 antibody under conditions permissive for binding of the anti-C16orf54 antibody to C16orf54, and detecting whether a complex is formed between the anti-C16orf54 antibody and C16orf54.

In one aspect, the invention provides a method of diagnosing a disorder associated with increased expression of C16orf54. In certain embodiments, the method comprises contacting a test cell with an anti-C16orf54 antibody; determining the level of expression (either quantitatively or qualitatively) of C16orf54 by the test cell by detecting binding of the anti-C16orf54 antibody to C16orf54; and comparing the level of expression of C16orf54 by the test cell with the level of expression of C16orf54 by a control cell (e.g., a normal cell of the same tissue origin as the test cell or a cell that expresses C16orf54 at levels comparable to such a normal cell), wherein a higher level of expression of C16orf54 by the test cell as compared to the control cell indicates the presence of a disorder associated with increased expression of C16orf54. In certain embodiments, the increased expression corresponds to higher density of C16orf54 expression on the surface of a tumor cell as compared to a normal cell. In certain embodiments, the test cell is obtained from an individual suspected of having a disorder associated with increased expression of C16orf54. In certain embodiments, the disorder is a cell proliferative disorder, such as a cancer or a tumor. Exemplary cell proliferative disorders that may be diagnosed using an antibody of the invention include a leukemia such as CLL, ALL, AML or CML, a lymphoma, a myeloma, a solid tumor such as breast cancer or pancreatic cancer, or metastases of any of those cancers.

In certain embodiments, a method of diagnosis or detection, such as those described above, comprises detecting binding of an anti-C16orf54 antibody to C16orf54 expressed on the surface of a cell or in a membrane preparation obtained from a cell expressing C16orf54 on its surface. In certain embodiments, the method comprises contacting a cell with an anti-C16orf54 antibody under conditions permissive for binding of the anti-C16orf54 antibody to C16orf54, and detecting whether a complex is formed between the anti-C16orf54 antibody and C16orf54 on the cell surface. An exemplary assay for detecting binding of an anti-C16orf54 antibody to C16orf54 expressed C16orf54 on the surface of a cell is a “FACS” assay.

Certain other methods can be used to detect binding of anti-C16orf54 antibodies to C16orf54. Such methods include, but are not limited to, antigen-binding assays that are well known in the art, such as western blots, radioimmunoassays, ELISA (enzyme linked immunosorbent assay), “sandwich” immunoassays, immunoprecipitation assays, fluorescent immunoassays, protein A immunoassays, and immunohistochemistry (IHC).

In certain embodiments, anti-C16orf54 antibodies are labeled. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

In certain embodiments, anti-C16orf54 antibodies are immobilized on an insoluble matrix. Immobilization entails separating the anti-C16orf54 antibody from any C16orf54 that remains free in solution. This conventionally is accomplished by either insolubilizing the anti-C16orf54 antibody before the assay procedure, as by adsorption to a water-insoluble matrix or surface (Bennich et al.., U.S. Pat. No. 3,720,760), or by covalent coupling (for example, using glutaraldehyde cross-linking), or by insolubilizing the anti-C16orf54 antibody after formation of a complex between the anti-C16orf54 antibody and C16orf54, e.g., by immunoprecipitation.

Any of the above embodiments of diagnosis or detection may be carried out using an immunoconjugate of the invention in place of or in addition to an anti-C16orf54 antibody.

Assays Anti-C16ORF54 antibodies and immunoconjugates of the invention may be characterized for their physical/chemical properties and/or biological activities by various assays known in the art.

Activity Assays

In one aspect, assays are provided for identifying anti-C16orf54 antibodies or immunoconjugates thereof having biological activity. Biological activity may include, e.g., the ability to inhibit cell growth or proliferation (e.g., “cell killing” activity), or the ability to induce cell death, including programmed cell death (apoptosis), or cell differentiation or cell activation. Antibodies or immunoconjugates having such biological activity in vivo and/or in vitro are also provided.

In certain embodiments, an anti-C16orf54 antibody or immunoconjugate thereof is tested for its ability to inhibit cell growth or proliferation in vitro. Assays for inhibition of cell growth or proliferation are well known in the art. Certain assays for cell proliferation, exemplified by the “cell killing” assays described herein, measure cell viability. One such assay is the CellTiter-Glo™ Luminescent Cell Viability Assay, which is commercially available from Promega (Madison, Wis.). That assay determines the number of viable cells in culture based on quantitation of ATP present, which is an indication of metabolically active cells. See Crouch et al. (1993) J. Immunol. Meth. 160:81-88, U.S. Pat. No. 6,602,677. The assay may be conducted in 96- or 384-well format, making it amenable to automated high-throughput screening (HTS). See Cree et al. (1995) AntiCancer Drugs 6:398-404.

Another assay for cell proliferation is the “MTT” assay, a colorimetric assay that measures the oxidation of 3-(4,5-dimethylthiazol-2-yl)-2,5-diphenyltetrazolium bromide to formazan by mitochondrial reductase. Like the CellTiter-Glo™ assay, this assay indicates the number of metabolically active cells present in a cell culture. See, e.g., Mosmann (1983) J. Immunol. Meth. 65:55-63, and Zhang et al. (2005) Cancer Res. 65:3877-3882.

In one aspect, an anti-C16orf54 antibody is tested for its ability to induce cell death in vitro. Assays for induction of cell death are well known in the art. In some embodiments, such assays measure, e.g., loss of membrane integrity as indicated by uptake of propidium iodide (PI), trypan blue (see Moore et al. (1995) Cytotechnology, 17:1-11), or 7AAD. In an exemplary PI uptake assay, cells are cultured in Dulbecco's Modified Eagle Medium (D-MEM): Ham's F-12 (50:50) supplemented with 10% heat-inactivated FBS (Hyclone) and 2 mM L-glutamine. Thus, the assay is performed in the absence of complement and immune effector cells. Cells are seeded at a density of 3×10⁶ per dish in 100×20 mm dishes and allowed to attach overnight. The medium is removed and replaced with fresh medium alone or medium containing various concentrations of the antibody or immunoconjugate. The cells are incubated for a 3-day time period. Following treatment, monolayers are washed with PBS and detached by trypsinization. Cells are then centrifuged at 1200 rpm for 5 minutes at 4° C., the pellet resuspended in 3 ml cold Ca²⁺ binding buffer (10 mM Hepes, pH 7.4, 140 mM NaCl, 2.5 mM CaC₁₂) and aliquoted into 35 mm strainer-capped 12×75 mm tubes (1 ml per tube, 3 tubes per treatment group) for removal of cell clumps. Tubes then receive PI (10 μg/ml). Samples are analyzed using a FACSCAN™ flow cytometer and FACSCONVERT™ CellQuest software (Becton Dickinson). Antibodies or immunoconjugates that induce statistically significant levels of cell death as determined by PI uptake are thus identified.

In one aspect, an anti-C16orf54 antibody or immunoconjugate is tested for its ability to induce apoptosis (programmed cell death) in vitro. An exemplary assay for antibodies or immunconjugates that induce apoptosis is an annexin binding assay, for example, as in Zhang et al. (BioTechniques 23: 525-531, 1997). Another exemplary assay for antibodies or immunconjugates that induce apoptosis is a histone DNA ELISA colorimetric assay for detecting internucleosomal degradation of genomic DNA. Such an assay can be performed using, e.g., the Cell Death Detection ELISA kit (Roche, Palo Alto, Calif.).

Cells for use in any of the above in vitro assays include cells or cell lines that naturally express C16orf54 or that have been engineered to express C16orf54. Such cells include tumor cells that overexpress C16ORF54 relative to normal cells of the same tissue origin. Such cells also include cell lines (including tumor cell lines) that express C16orf54 and cell lines that do not normally express C16orf54 but have been transfected with nucleic acid encoding C16orf54.

In one aspect, an anti-C16orf54 antibody or immunoconjugate thereof is tested for its ability to inhibit cell growth or proliferation in vivo. In certain embodiments, an anti-C16orf54 antibody or immunoconjugate thereof is tested for its ability to inhibit tumor growth in vivo. In vivo model systems, such as xenograft models, can be used for such testing. In an exemplary xenograft system, human tumor cells are introduced into a suitably immunocompromised non-human animal, e.g., a SCID mouse. An antibody or immunoconjugate of the invention is administered to the animal. The ability of the antibody or immunoconjugate to inhibit or decrease tumor growth is measured. In certain embodiments of the above xenograft system, the human tumor cells are tumor cells from a human patient. Such cells useful for preparing xenograft models include without limitation cells expressing exogenous C16orf54, and cells naturally expressing C16orf54. In certain embodiments, the human tumor cells are introduced into a suitably immunocompromised non-human animal by subcutaneous injection or by transplantation into a suitable site, such as a mammary fat pad.

Binding Assays and Other Assays

In one aspect, an anti-C16orf54 antibody is tested for its antigen binding activity. For example, in certain embodiments, an anti-C16orf54 antibody is tested for its ability to bind to exogenous or endogenous C16orf54 expressed on the surface of a cell. A FACS assay may be used for such testing.

A panel of monoclonal antibodies raised against C16orf54 may be grouped based upon the epitiopes they recognize, a process known as epitope binning. Epitope binning is typically carried out using competition assays, which evaluate an antibody's ability to bind to an antigen in the presence of another antibody. In an exemplary competition assay, immobilized C16orf54 is incubated in a solution comprising a first labeled antibody that binds to C16orf54 and a second unlabeled antibody that is being tested for its ability to compete with the first antibody for binding to C16orf54. The second antibody may be present in a hybridoma supernatant. As a control, immobilized C16orf54 is incubated in a solution comprising the first labeled antibody but not the second unlabeled antibody. After incubation under conditions permissive for binding of the first antibody to C16orf54, excess unbound antibody is removed, and the amount of label associated with immobilized C16orf54 is measured. If the amount of label associated with immobilized C16orf54 is substantially reduced in the test sample relative to the control sample, then that indicates that the second antibody is competing with the first antibody for binding to C16orf54. In certain embodiments, immobilized C16orf54 is present on the surface of a cell or in a membrane preparation obtained from a cell expressing C16orf54 on its surface.

High-throughput methods of epitope binning are also known in the art. See, for example, Jia et al., J. Immunol. Methods 2004, 288(1-2):91-98, describing a method of multiplexed competitive antibody binning for the characterization of monoclonal antibodies; and Miller et al., J. Immunol. Methods 2011, 365(1-2):118-25, describing epitope binning of murine monoclonal antibodies by a multiplexed pairing assay.

Epitope Mapping

Epitope mapping is the process of identifying the binding sites, or epitopes, of an antibody on its target protein antigen. Antibody epitopes may be linear epitopes or conformational epitopes. Linear epitopes are formed by a continuous sequence of amino acids in a protein. Conformational epitopes are formed of amino acids that are discontinuous in the protein sequence, but which are brought together upon folding of the protein into its three-dimensional structure.

A variety of methods are known in the art for mapping antibody epitopes on target protein antigens. These include mutagenesis methods, peptide scanning methods, display methods, methods involving and mass spectroscopy, and structural determination.

The site directed mutagenesis method involves targeted site-directed mutagenesis where critical amino acids are identified by systematically introducing substitutions along the protein sequence and then determining the effects of each substitution on antibody binding. This may be done by “alanine scanning mutagenesis,” as described by Cunningham and Wells (1989) Science 244: 1081-1085, or some other form of point mutagenesis of amino acid residues in human C16orf54. Mutagenesis studies, however, may also reveal amino acid residues that are crucial to the overall three-dimensional structure of C16orf54 but that are not directly involved in antibody-antigen contacts, and thus other methods may be necessary to confirm a functional epitope determined using this method.

Shotgun mutagenesis mapping utilizes a comprehensive plasmid-mutation library for the target gene, with each clone in the library bearing a unique amino acid mutation and the entire library covering every amino acid in the target protein. The clones that constitute the mutation library are individually arranged in microplates, expressed within living mammalian cells, and tested for immunoreactivity with antibodies of interest. Amino acids critical for antibody epitopes are identified by a loss of reactivity and are then mapped onto a protein structure to visualize epitopes. By automating the analysis, new epitope maps can be derived within days to weeks. Because it uses the native structure of proteins within mammalian cells, the technique allows both linear and conformational epitope structures to be mapped on complex proteins. (Paes et al., J. Am. Chem. Soc. 131(20): 6952-6954 (2009); Banik and Doranz, Genetic Engineering and Biotechnology News 3(2): 25-28 (2010)).

The epitope bound by an anti-C16orf54 antibody may also be determined using peptide scanning methods. In peptide scanning, libraries of short peptide sequences from overlapping segments of the target protein, C16orf54, are tested for their ability to bind antibodies of interest. The peptides are synthesized and screened for binding, e.g., using ELISA or BIACORE, or on a chip, by any of the multiple methods for solid-phase screening (Reineke et al., Curr. Opin. Biotechnol. 12: 59-64, 2001) as in the “pepscan” methodology (WO 84/03564; WO 93/09872). Such peptide screening methods may not be capable of detecting some discontinuous functional epitopes, i.e. functional epitopes that involve amino acid residues that are not contiguous along the primary sequence of the C16orf54 polypeptide chain.

A recently developed technology termed CLIPS (chemical linkage of peptides onto scaffolds) may be used to map conformational epitopes. The loose ends of the peptides are affixed onto synthetic scaffolds, so that the scaffolded peptide may be able to adopt the same spatial structure as the corresponding sequence in the intact protein. CLIPS technology is used to fix linear peptides into cyclic structures (‘single-loop’ format), and to bring together different parts of a protein binding site (‘double-loop’, ‘triple-loop’, etc. format), so as to create conformational epitopes that may be assayed for antibody binding. (U.S. Pat. No. 7,972,993).

The epitopes bound by antibodies of the invention may also be mapped using display techniques, including, for example, phage display, microbial display, and ribosome/mRNA display as described above. In these methods, libraries of peptide fragments are displayed on the surface of the phage or cell. Epitopes are then mapped by screening mAbs against these fragments using selective binding assays. A number of computational tools have been developed which allow the prediction of conformational epitopes based upon linear affinity-selected peptides obtained using phage display. (Mayrose et al., Bioinformatics 23: 3244-3246, 2007). Methods are also available for the detection of conformational epitopes by phage display. Microbial display systems may also be used to express properly folded antigenic fragments on the cell surface for identification of conformational epitopes (Cochran et al., J. Immunol. Meth. 287: 147-158, 2004; Rockberg et al., Nature Methods 5: 1039-1045, 2008).

Methods involving proteolysis and mass spectroscopy may also be used to determine antibody epitopes (Baerga-Ortiz et al., Protein Sci. 2002 June; 11(6): 1300-1308). In limited proteolysis, the antigen is cleaved by different proteases, in the presence and in the absence of the antibody, and the fragments are identified by mass spectrometry. The epitope is the region of the antigen that becomes protected from proteolysis upon binding of the antibody (Suckau et al., Proc. Natl. Acad. Sci. USA 87:9848-9852, 1990). Additional proteolysis based methods include, for example, selective chemical modification (Fiedler et al., Bioconjugate Chemistry 1998, 9(2): 236-234, 1998), epitope excision (Van de Water et al., Clin. Immunol. Immunopathol. 1997, 85(3): 229-235, 1997), and the recently developed method of hydrogen-deuterium (H/D) exchange (Flanagan, N., Genetic Engineering and Biotechnology News 3(2): 25-28, 2010).

The epitope bound by antibodies of the present invention may also be determined by structural methods, such as X-ray crystal structure determination (e.g., WO 2005/044853), molecular modeling and nuclear magnetic resonance (NMR) spectroscopy, including NMR determination of the H-D exchange rates of labile amide hydrogens when free and when bound in a complex with an antibody of interest (Zinn-Justin et al. (1992) Biochemistry 31:11335-11347; Zinn-Justin et al. (1993) Biochemistry 32:6884-6891).

Additional antibodies binding to the same epitope as an antibody of the present invention may be obtained, for example, by screening of antibodies raised against C16orf54 for binding to the epitope, by immunization of an animal with a peptide comprising a fragment of human C16ORF54 comprising the epitope sequence, or by selection of antibodies using phage display for binding to the epitope sequence. Antibodies that bind to the same functional epitope might be expected to exhibit similar biological activities, such as blocking a biological activity of C16ORF54, and such activities can be confirmed by functional assays of the antibodies.

Additional Activity Assays

In one embodiment, an anti-C16orf54 antibody of the invention is an antagonist antibody that inhibits a biological activity of C16orf54. The anti-C16orf54 antibodies of the invention may be assayed to determine if they inhibit a biological activity of C16orf54.

In one aspect, purified anti-C16orf54 antibodies can be further characterized by a series of assays including, but not limited to, N-terminal sequencing, amino acid analysis, non-denaturing size exclusion high pressure liquid chromatography (HPLC), mass spectrometry, ion exchange chromatography and papain digestion.

In one embodiment, the invention contemplates an altered antibody that possesses some but not all effector functions, which make it a desirable candidate for many applications in which the half life of the antibody in vivo is important yet certain effector functions (such as complement and ADCC) are unnecessary or deleterious. In certain embodiments, the Fc activities of the antibody are measured to ensure that only the desired properties are maintained. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. An example of an in vitro assay to assess ADCC activity of a molecule of interest is described in U.S. Pat. No. 5,500,362 or 5,821,337. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1998). C1q binding assays may also be carried out to confirm that the antibody is unable to bind C1q and hence lacks CDC activity. To assess complement activation, a CDC assay, e.g., as described in Gazzano-Santoro et al., J. Immunol. Methods 202:163 (1996), may be performed. FcRn binding and in vivo clearance/half life determinations can also be performed using methods known in the art.

Although the foregoing invention has been described in some detail by way of illustration and example for purposes of clarity of understanding, the descriptions and examples should not be construed as limiting the scope of the invention. The disclosures of all patent and scientific literatures cited herein are expressly incorporated in their entirety by reference.

### EXAMPLES

The following are examples of methods and compositions of the invention. It is understood that various other embodiments may be practiced, given the general description provided above.

### Example 1

### Identification of C16orf54 on the Surface of Chronic Lymphocytic Leukemia (CLL) Tumor Cells

A total of 33 CLL patient samples and 22 normal samples from healthy donors were initially analyzed. To monitor the quality of individual CLL samples, expression of CD19, CD5 and CD23 was assessed using LC-MS/MS or FACS. Only samples containing at least 75% positive cells were further analyzed. Freshly harvested primary tumor and normal samples were used so as to maximally maintain cell viability during cell surface protein profiling. Optimal labeling times for tumor and normal samples were determined to allow for efficient labeling without compromise of cellular integrity.

Surface tagged antigen profiling (sTAg) was used to identify and quantitatively profile the repertoire of surface proteins on cells in 33 core CLL samples, 11 bone marrow mononuclear cell (BMMC) control and 11 peripheral blood mononuclear cell (PBMC) control samples. The extracellular domains of proteins associated with the cell membranes of intact primary tumor cells were chemically tagged, and then chromatographically enriched for tagged proteins using a solid-phase affinity resin. Eluted proteins were stored at −80° C. prior to mass spectrometry analysis as described below.

Proteins enriched by the sTAg method were identified and quantified using high-resolution, shotgun liquid chromatography tandem mass spectrometry. A hybrid ThermoFisher Orbitrap® Velos hybrid MS instrument, which combines the sensitivity of a linear ion trap with the high-resolution and mass accuracy afforded by the revolutionary orbitrap mass analyzer (Olsen et al., Mol. Cell Proteomics 8:2759-2769, 2009) was coupled to a nanoflow liquid chromatography apparatus, and employed for shotgun-based, bottoms-up proteomics to determine the identities and quantitative abundance measurements of proteins in the CLL cell surface enrichment fractions (Yates et al. (2009) Annu. Rev. Biomed. Eng. 11: 49-79). Enriched surface proteins were proteolytically digested to peptides by trypsin, then separated by hydrophobicity via nanoflow liquid chromatography, and peptide fragmentation patterns were recorded dynamically by the MS. Subsequent processing of the raw MS data was carried out using the SEQUEST algorithm executed on a fast-processing Sorcerer 2 platform (Lundgren et al., 2009) Curr. Protoc. Bioinformatics, Chapter 13: Unit 13.3), then matched to experimental fragmentation patterns to all possible theoretical patterns determined in-silico from the human proteome to determine peptide and protein identities. Resulting matches were statistically validated using PeptideProphet® (Keller et al. (2002) Anal. Chem. 74: 5383-5392) and Protein Prophet® (Nesvizhskii et al. (2003) Anal. Chem. 75: 4646-4658) software tools to ensure the lowest possible false discovery rates (FDR) and thus inclusion of only robustly identified proteins in the candidate pool.

The relative quantitative levels of identified proteins in the sTAg samples were determined using the spectral counting method (Neilson et al., Proteomics 11:535-553, 2011). Spectral counting is based on the empirical demonstration that the number of assigned (positively identified) spectra associated with peptides from each protein correlates strongly with that protein's relative abundance in the original mixture (Liu et al., Anal. Chem. 76:4193-4201, 2004). Spectral counts were obtained from proteomics analytical software Scaffold® (Proteome Software) that displays, sorts and filters the results of SEQUEST-searched mass spectrometry data. Raw spectral counts were transformed to Percent Normalized Spectral Abundance Factor (% NSAF) values (Zybailov et al. J. Proteome Res. 5: 2339-2347, 2006) to account for differences in protein length and variability in sample size.

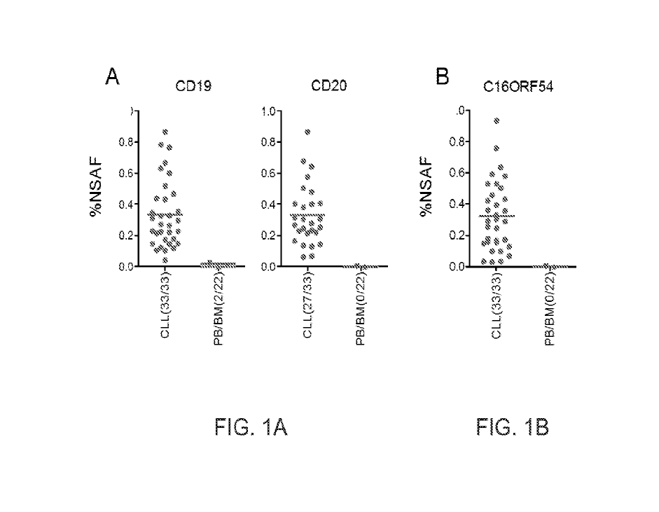

Using sTAg, both known therapeutic antibody targets (CD19 and CD20) and novel target C16orf54 were identified as being present at high density on the surface of CLL tumor cells. As plotted in FIG. 1A, the sTAg method detected CD19 in 33 of 33 CLL samples with a mean % NSAF of 0.34, and CD20 in 27 of 33 CLL samples with a mean % NSAF of 0.32. In the control samples CD19 was detected in 2 of 22 PBMC and BMMC samples with a mean % NSAF of 0.015 and CD20 was not detected. C16orf54, a single-pass membrane protein having the amino acid sequence of SEQ ID NO: 1, was identified in 33 of 33 primary CLL samples with a mean % NSAF of 0.32 but not in any of 22 normal samples (FIG. 1B). Based on this analysis, C16orf54 is substantially enriched on a significant portion of patient-derived CLL primary tumor specimens relative to relevant normal controls.

Additional tumor patient samples and normal samples from healthy donors were analyzed. As shown in Table 30 below, based on sTAg analysis, C16orf54 is expressed in some primary samples of acute myeloid leukemia (AML) tumor cells (4/14) and multiple myeloma (MM) tumor cells (1/30), as well as nearly all primary samples of CLL tumor cells tested (36/40, including the 33 samples discussed above), but not on colorectal (CRC) tumor samples (0/27), lung tumor samples (0/89), ovary tumor samples (0/53), sarcoma tumor samples (0/14) or pancreas tumor samples (0/41).

TABLE 30

| | | | Expression by sTAg |
|---|---|---|---|
| Indication | T/N | Prevalence | (Median % NSAF) |
| AML | Tumor | 4/14 | 0.084 |
| | BMMC | 0/16 | 0.000 |
| CLL | Tumor | 36/40 | 0.272 |
| | PBMC | 0/15 | 0.022 |
| MM | Tumor | 1/30 | 0.110 |
| | BMMC | 0/16 | 0.000 |
| CRC | Tumor | 0/27 | 0.000 |
| | NAT | 0/22 | 0.000 |
| Lung | Tumor | 0/89 | 0.000 |
| | NAT | 0/75 | 0.000 |
| Ovary | Tumor | 0/53 | 0.000 |
| | NAT | 0/10 | 0.000 |
| Sarcoma | Tumor | 0/14 | 0.000 |
| | NAT | nd | 0.000 |
| Pancreas | Tumor | 0/41 | 0.000 |
| | NAT | 0/21 | 0.000 |

NAT = normal adjacent tissue

### Example 2

### Preparation of Monoclonal Antibodies to C16orf54

Monoclonal antibodies were prepared in accordance with a general method as described in “Antibodies: A Laboratory Manual” (Harlow and Lane 1988 CSH Press). Male 129S6 mice purchased from Taconic were used for immunization. Mice were immunized via subcutaneous injection in the flank with 1.5×10⁶ human C16orf54 (huC16orf54) expressing sarcoma cells. On day 55 post-immunization, mice were boosted intraperitoneally with 5×10⁶ huC16orf54-expressing sarcoma cells. Spleens were harvested on day 59. Individual splenocytes were prepared and fused with CRL-2016 myeloma cells (ATCC) using a PEG-based method as generally described in “Antibodies: A Laboratory Manual” (Harlow and Lane 1988 CSH Press) to establish hybridomas.

Hybridomas were grown in 384 well tissue culture plates and supernatants from individual wells were screened by ELISA for production of antibodies recognizing huC16orf54. Positive wells were then transferred to 96 well plates, expanded, and supernatants were collected for huC16orf54 binding confirmation by ELISA. Individual hybridomas producing anti-huC16orf54 antibodies were established as confirmed unique clones producing monoclonal anti-huC16orf54 antibodies by plating single hybridoma cells in wells of 96 well plates. These cells were grown into colonies and the supernatant from these individual colonies was screened by ELISA to confirm monoclonal antibody binding to huC16orf54. Eight monoclonal antibodies (designated R29-67-1B, R29-7-2A, R29-67-4A, R29-67-7A, R29-67-3C, R29-7-1C, R29-67-9A, and R29-67-5A) were selected for their binding to C16orf54 and further analyzed in vitro and in vivo as described herein. Clonal hybridomas were injected into pristane treated Balb/C mice to produce ascites. Ascites was collected and purified using Gammabind sepharose (GE Healthcare product code 17-0885-01), Protein A IgG binding buffer (Thermo Scientific part number 21001), and IgG elution buffer (Thermo Scientific part number 21004) following the general antibody purification protocol published by Thermo Scientific (Product Instructions #21001).

The nucleic acid and amino acid sequences for the heavy chain and light chain variable regions of the antibody R29-7-2A are shown below:

R29-7-2A heavy chain variable region

(SEQ ID NO: 3)

ATGCTGTTGGGGCTGAAGTGGGTTTTCTTTGTTGTTTTTTATCAAGGTGT
GCCTTGTGAGGTGCAGCTTGTTGAGTCTGGTGGAGGATTGATACAGCCTA
AAGGGTCATTGAAACTCTCATGTGTAGCCTCTGGATTCTCCTTCAATACC
CACGCCATGAACTGGGTCCGTCAGGCTCCAGGAAAGGGTTTGGAATGGGT
TGCTCGCATAAGAAGTAAAAGTAATAATTATGCAAGATATTATGCCGATT
CAGTGAAAGACAGGTTCACCATCTCCAGAGATGATTCACAAAGCATTCTC
TATCTGCAAATGAACAACTTGAAAACTGAGGACACAGCCATGTATTACTG
TGTAAAACAGGGGGACGGGGGGTTTGCTTACTGGGGCCAAGGGACCCTGG
TCACTGTCTCTGCA

(SEQ ID NO: 4; exemplary CDRs are underlined;
see also, Table 29)

MLLGLKWVFFVVFYQGVPCEVQLVESGGGLIQPKGSLKLSCVASGFSFNT
HAMNWVRQAPGKGLEWVARIRSKSNNYARYYADSVKDRFTISRDDSQSIL
YLQMNNLKTEDTAMYYCVKQGDGGFAYWGQGTLVTVSA
R29-7-2A light chain variable region

(SEQ ID NO: 5)

ATGGATTCACAGGCCCAGGTTCTTATGTTACTGCTGCTATGGGTATCTGG
TACCTGTGGGGACATTGTGATGTCACAGTCTCCATCCTCCCTAGCTGTGT
CAGTTGGAGAGAAGGTTACTATGAGCTGCAAGTCCAGTCAGAGCCTTTTA
TATAGTAGCAATCAAAAGAACTACTTGGCCTGGTACCAGCAGAGACCAGG
GCAGTCTCCTAAACTACTGATTTACTGGGCATCCGCTAGGGAATCTGGGG
TCCCTGATCGCTTCACAGGCAGTGGATCTGGGACAGATTTCACTCTCACC
ATCAGCAGTGTGAAGGCTGAAGACCTGGCAGTTTATTACTGTCAGCAATA
TTATAGCTATCCTCCCACGTTCGGTGCTGGGACCAAGGTGGAGCTGAAA

(SEQ ID NO: 6; exemplary CDRs are underlined;
see also, Table 29)

MDSQAQVLMLLLLWVSGTCGDIVMSQSPSSLAVSVGEKVTMSCKSSQSLL
YSSNQKNYLAWYQQRPGQSPKLLIYWASARESGVPDRFTGSGSGTDFTLT
ISSVKAEDLAVYYCQQYYSYPPTFGAGTKVELK

The nucleic acid and amino acid sequences for the heavy chain and light chain variable regions of the antibody R29-7-1C are shown below:

R29-7-1C heavy chain variable region

(SEQ ID NO: 7)

ATGAGAGTGCTGATTCTTTTGTGGCTGTTCACAGCCTTTCCTGGTATCCT
GTCTGATGTGCAGCTTCAGGAGTCGGGACCTGGCCTGGTGAAACCTTCTC
AGTCTCTGTCCCTCACCTGCACTGTCACTGGCTACTCAATCACCAGTGAT
TATGCCTGGAACTGGATCCGGCAGTTTCCAGGAAACAAACTGGAGTGGAT
GGGCTACATAAGCTACAGTGGTAGTATTAGGTACAACCCATCTCTCAAAA
GTCGAATCTCTATCACTCGAGACACATCCAAGAACCAGTTCTTCCTGCAG
TTGAATTCTGTGACTACTGAGGACACAGCCACATATTACTGTGCAAGAGA
GAAGTATGATAACTATTACTATGCTATGGACTACTGGGGTCAAGGAACCT
CAGTCACCGTCTCCTCA

(SEQ ID NO: 8; exemplary CDRs are underlined;
see also, Table 21)

MRVLILLWLFTAFPGILSDVQLQESGPGLVKPSQSLSLTCTVTGYSITSD
YAWNWIRQFPGNKLEWMGYISYSGSIRYNPSLKSRISITRDTSKNQFFLQ
LNSVTTEDTATYYCAREKYDNYYYAMDYWGQGTSVTVSS
R29-7-1C light chain variable region

(SEQ ID NO: 9)

ATGGATTCACAGGCCCAGGTTCTTATGTTACTGCTGCTATGGGTATCTGG
TACCTGTGGGGACATTGTAATGTCACAGTCTCCATCCTCCCTAGCTGTGT
CAGTTGGAGAGAAGGTTACTCTGAGCTGCAAGTCCAGTCAGAACCTTTTA
TATAGTACCAATCAAAAGAACTACTTGGCCTGGTACCAGCAGAAACCAGG
GCAGTCTCCTAAACTGCTGATTTACTGGGCATCCACTAGGGAATCTGGGG
TCCCTGATCGCTTCACAGGCAGTGGATCTGGGACAGATTTCACTCTCACC
ATCAGCAGTGTGAAGGCTGAGGACCTGGCAGTTTATTACTGTCAGCAATA
TTATAGCTATCGGACGTTCGGTGGAGGCACCAAGCTGGAAATCAAA

(SEQ ID NO: 10; exemplary CDRs are underlined;
see also, Table 21)

MDSQAQVLMLLLLWVSGTCGDIVMSQSPSSLAVSVGEKVTLSCKSSQNLL
YSTNQKNYLAWYQQKPGQSPKLLIYWASTRESGVPDRFTGSGSGTDFTLT
ISSVKAEDLAVYYCQQYYSYRTFGGGTKLEIK

The nucleic acid and amino acid sequences for the heavy chain and light chain variable regions of the antibody R29-67-7A are shown below:

R29-67-7A heavy chain variable region

(SEQ ID NO: 11)

ATGAGAGTGCTGATTCTTTTGTGGCTGTTCACAGCCTTTCCTGGTATCCT
GTCTGATGTGCAGCTTCAGGAGTCGGGACCTGGCCTGGTGAAACCTTCTC
AGTCTCTGTCCCTCACCTGCACTGTCACTGGCTACTCAATCACCAGTGAT
TATGCCTGGAACTGGATCCGGCAGTTTCCAGGAAACAAACTGGAGTGGAT
GGGCTACATAAACTACAGTGGTCGCACTAGATATAACCCATCTCTCAATA
GTCGCATCTCTCTCACTCGAGACACATCCACGAACCAGTTCTTCCTGCAG
TTGAATTCTGTGACTACTGAGGACACAGCCACATATTACTGTGCAAGAGA
GAACTATGATTACGAGTTTTATGCTATGGACTACTGGGGTCAAGGAACCT
CA GTCACCGTCTCCTCA

(SEQ ID NO: 12; exemplary CDRs are underlined;
see also, Table 22)

MRVLILLWLFTAFPGILSDVQLQESGPGLVKPSQSLSLTCTVTGYSITSD
YAWNWIRQFPGNKLEWMGYINYSGRTRYNPSLNSRISLTRDTSTNQFFLQ
LNSVTTEDTATYYCARENYDYEFYAMDYWGQGTSVTVSS
R29-67-7A light chain variable region

(SEQ ID NO: 13)

ATGGAATCACAGGCCCAGGTTCTTATGTTACTGCTGCTATGGGTATCTGG
TACCTGTGGGGACATTGTGATGTCACAGTCTCCATCCTCCCTAGCTGTGT
CAGTTGGAGAGAAGGTTACTATGAGCTGCAAGTCCAGTCAGAACCTTTTA
TATAGTAGCAATCAAAAGAACTACTTGGCCTGGTACCAGCAGAAACCAGG
GCAGTCTCCTAAACTGCTGATTTACTGGGCATCCACTAGGGAATCTGGGG
TCCCTGATCGCTTCACAGGCAGTGGATCTGGGACAGATTTCACTCTCACC
ATCAGCAGTGTGAAGGCTGAAGACCTGGCAGTTTATTACTGTCAGCAATA
TTATATCTATCGGACGTTCGGTGGAGGCACCAAGCTGGAAATCAAA

(SEQ ID NO: 14; exemplary CDRs are underlined;
see also, Table 22)

MESQAQVLMLLLLWVSGTCGDIVMSQSPSSLAVSVGEKVTMSCKSSQNLL
YSSNQKNYLAWYQQKPGQSPKLLIYWASTRESGVPDRFTGSGSGTDFTLT
ISSVKAEDLAVYYCQQYYIYRTFGGGTKLEIK

The nucleic acid and amino acid sequences for the heavy chain variable region of the antibody R29-67-4A are shown below:

R29-67-4A heavy chain variable region

(SEQ ID NO: 145)

TCTGATGTGCAACTTCAGGAGTCGGGACCTGGCCTGGTGAAACCTTCTC
AGTCTCTGTCCCTCACCTGCACTGTCACTGGCTACTCAATCACCAGTGTT
TATGCCTGGAACTGGATCCGGCAGTTTCCAGGAAACAAACTGGAGTGGAT
GGGCTACATAAGCTACAGTGGTATCACTACCTACAACCCATCTCTCAAAA
GTCGAATCTCTATCACTCGAGACACATCCAAGAACCAGTTCTTCCTGCAG
TTGAATTCTGTGACTACTGAGGACACAGCCACATATTACTGTGCAACAAC
TGGGACCCGCTGGGGCCAAGGCACCACTCTCACAGTCTCCTCA

(SEQ ID NO: 146; exemplary CDRs are underlined;
see also, Table 23)

SDVQLQESGPGLVKPSQSLSLTCTVTGYSITSVYAWNWIRQFPGNKLEWM
GYISYSGITTYNPSLKSRISITRDTSKNQFFLQLNSVTTEDTATYYCATT
GTRWGQGTTLTVSS

Exemplary light chain variable region sequences suitable for use in conjunction with the R29-67-4A heavy chain variable region sequences shown above for preparation of C16orf54 binding antibodies comprising heavy chain and light chain variable region sequences include: SEQ ID NO:21 or SEQ ID NO:22 (see, e.g., R29-8-57B antibody light chain variable region sequences); SEQ ID NO:9 or SEQ ID NO:10 (see, e.g., R29-7-1C antibody light chain variable region sequences); SEQ ID NO:13 or SEQ ID NO:14 (see, e.g., R29-67-7A antibody light chain variable region sequences).

### Example 3

### Preparation of Additional Monoclonal Antibodies to C16orf54

Antibodies to C16orf54 were generated using the invitro anti-tumor Antibody (iTAb) platform. In this system, a mouse tumor cell line is transduced to stably express the human protein and then implanted subcutaneously in syngeneic mice. The mice are treated with anti-CD8 antibody to remove the cell mediated rejection pathway while leaving the humoral immune response intact. Following this immunization, spenocytes are harvested, and are fused to an immortalized partner cell to generate hybridomas. Antibodies from these hybridomas are screened in multiple assays designed to identify a diverse panel of antibodies with good binding properties. The selected antibodies are then produced for in vivo testing as follows.

Murine sarcoma cell lines that express C16orf54 were prepared by virus infection of sarcoma cell lines. A PCR-amplified C16orf54 gene was cloned into a murine stem cell virus expression vector with a neomycin resistance gene and sequenced to confirm the identity. To prepare virus particles, HEK 293t cells with retroviral packaging proteins were transfected, in the presence of transfection reagent FUGENE HD (Roche), with the retroviral expression vector containing C16orf54. The virus particles collected from the supernatant of the culture media 48 hours after transfection were used to infect the sarcoma cells. After G418 selection, stable transfectants were pooled and then cloned by limiting dilution. Clones were then picked and expanded in the presence of antibiotics. Clones with the highest expression level of C16orf54 as measured by flow cytometry were expanded and banked. These cell lines were then used to immunize the syngeneic mice for antibody production and in the binding assays for antibody selection as follows.

For immunization, the mouse sarcoma cell line that expresses C16orf54 was implanted subcutaneously in 129s6/SvEv mice, which are syngeneic with the sarcoma line. Mice were boosted with the cell line three days prior to spleen harvest. Splenocytes were isolated as single cells and fused with SP2-MIL6 cells using PEG1500. Resulting hybridomas were plated in 384-well plates and allowed to grow for ten days.

Antibodies against C16orf54 were initially selected using a cell-based enzyme-linked immunosorbant assay (ELISA) to detect binding to C16orf54. For this assay, the C16orf54 expressing sarcoma cells were plated in 384-well plates one day prior to assay. Cells were then treated with hybridoma supernatants. Following incubation and wash, the presence of bound antibody was detected using a peroxidase-conjugated goat anti-mouse IgG antibody (Jackson ImmunResearch Laboratories) followed by a chemiluminescent substrate (ThermoScientific SuperSignal® ELISA Pico Substrate). Hybridomas identified as positive in the initial screen were transferred to the wells of a 96-well plate. After growth, the supernatants were tested in a similar assay for confirmation.

The isotype of the antibodies was identified by ELISA by using isotype specific goat anti-mouse Fc antibodies. For this assay, C16orf54 expressing cells were plated in 384-well plates one day prior to assay. Cells were then treated with hybridoma supernatants. Following incubation and wash, cells were incubated with peroxidase-conjugated goat antibody specific for mouse IgG1 or IgG2a (Jackson ImmunResearch Laboratories), followed by a chemiluminescent substrate (ThermoScientific SuperSignal® ELISA Pico Substrate).

Concentration of antibody in supernatants found to be positive for binding to the C16orf54 expressing cells was measured by ELISA. Supernatants were tested at four dilutions. For each antibody, the dilution that generated a value within the linear range of the standard curve was used to calculate the concentration of the antibody in the supernatant. Antibody concentration in the supernatants ranged in concentration from <1 μg/ml to >500 μg/ml, with approximately 300 supernatants >10 μg/ml. Twenty-five monoclonal antibodies (designated R29-8-9B, R29-8-93B, R29-8-51B, R29-8-30A, R29-8-120B, R29-8-18B, R29-8-28C, R29-8-19B, R29-8-50C, R29-8-12A, R29-8-36C, R29-8-58C, R29-8-75B, R29-8-57B, R29-8-136C, R29-67-4A, R29-7A-53A, R29-7A-54C, R29-7A-38C, R29-7A-49A, R29-7A-13A, R29-67-1B, R29-67-3C, R29-67-5A, and R29-67-9A) were selected for their binding to C16orf54 and further analyzed in vitro and in vivo as described herein.

The nucleic acid and amino acid sequences for the heavy chain and light chain variable regions of the antibody R29-8-136C are shown below:

R29-8-136C heavy chain variable region

(SEQ ID NO: 15)

ATGAGAGTGCTGATTCTTTTGTGGCTGTTCACAGCCTTTCCTGGTATCCT
GTCTGATGTGCAGCTTCAGGAGTCGGGACCTGGCCTGGTGAAACCTTCTC
AGTCTCTGTCCCTCACCTGCACTGTCACTGGCTACTCAATCACCAGTGAT
TATGCCTGGAACTGGATCCGGCAGTTTCCAGGAAACAAACTGGAGTGGAT
GGGCTACATAAACTATAGTGGTAGTAGTAGGTACAACCCATCTCTCAAAA
GTCGAATCTCTATCACTCGAGACACATCCAAGAACCACTTCTTCCTGCAG
TTGAATTCTGTGACTCCTGAGGACACAGCCACATATCATTGTGCAAGAGA
GAGGTACGACGGGGTTTATTATGGTATGGACTACTGGGGTCAAGGAACCT
CAGTCACCGTGTCCTCA

(SEQ ID NO: 16; exemplary CDRs are underlined;
see also, Table 20)

MRVLILLWLFTAFPGILSDVQLQESGPGLVKPSQSLSLTCTVTGYSITSD
YAWNWIRQFPGNKLEWMGYINYSGSSRYNPSLKSRISITRDTSKNHFFLQ
LNSVTPEDTATYHCARERYDGVYYGMDYWGQGTSVTVSS
R29-8-136C light chain variable region

(SEQ ID NO: 17)

ATGGATTCACAGGCCCAGGTTCTTATGTCACTGCTGCTATGGGTATCTGG
TACCTGTGGGGACATTGTGATGTCACAGTCTCCATCCTCCCTAGCTGTGT
CAGTTGGAGAGAAGGTTACTATGAGCTGCAAGTCCAGTCAGAGCCTTTTA
TATAGTAGCAATCAAAAGAACTACTTGGCCTGGTACCAGCAGAAACCAGG
GCAGTCTCCTAAACTGCTGATTTACTGGGCATCCACTAGGGAATCTGGGG
TCCCTGATCGCTTCACAGGCAGTGGATCTGGGACAGATTTCACTCTCACC
ATCAGCAGTGTGAAGACTGAAGACCTGGCAGTTTATTACTGTCAGCAATA
TTATAGCTATCGGACGTTCGGTGGAGGCACCAAGCTGGAAATCAAA

(SEQ ID NO: 18; exemplary CDRs are underlined;
see also, Table 20)

MDSQAQVLMSLLLWVSGTCGDIVMSQSPSSLAVSVGEKVTMSCKSSQSLL
YSSNQKNYLAWYQQKPGQSPKLLIYWASTRESGVPDRFTGSGSGTDFTLT
ISSVKTEDLAVYYCQQYYSYRTFGGGTKLEIK

The nucleic acid and amino acid sequences for the heavy chain and light chain variable regions of the antibody R29-8-57B are shown below:

R29-8-57B heavy chain variable region

(SEQ ID NO: 19)

ATGAGAGTGCTGATTCTTTTGTGGCTGTTCACAGCCTTTCCTGGTATCCT
GTCTGATGTGCAGCTTCAGGAGTCGGGACCTGGCCTGGTGAAACCTTCTC
AGTCTCTGTCCCTCACCTGCACTGTCACTGGCTACTCAATCACCAGTGAT
TATGCCTGGAACTGGATCCGGCAGTTTCCAGGAAACAAACTGGAGTGGAT
GGGCTACATAAACTATAGTGGTAGTACTAGGTACAACCCATCTCTCAAAA
GTCGAATCTCTATCACTCGAGACACATCCAAGAACCACTTCTTCCTGCAG
TTGAATTCTGTGACTCCTGAGGACACAGCCACATATCATTGTGCAAGAGA
GAGGTACGACGGGGTTTATTATGGTATGGACTACTGGGGTCAAGGAACCT
CAGTCACCGTGTCCTCA

(SEQ ID NO: 20; exemplary CDRs are underlined;
see also, Table 19)

MRVLILLWLFTAFPGILSDVQLQESGPGLVKPSQSLSLTCTVTGYSITSD
YAWNWIRQFPGNKLEWMGYINYSGSTRYNPSLKSRISITRDTSKNHFFLQ
LNSVTPEDTATYHCARERYDGVYYGMDYWGQGTSVTVSS
R29-8-57B light chain variable region

(SEQ ID NO: 21)

ATGGATTCACAGGCCCAGGTTCTTATGTCACTGCTGCTATGGGTATCTGG
TACCTGTGGGGACATTGTGATGTCACAGTCTCCATCCTCCCTAGCTGTGT
CAGTTGGAGAGAAGGTTACTATGAGCTGCAAGTCCAGTCAGAGCCTTTTA
TATAGTAGCAATCAAAAGAACTACTTGGCCTGGTACCAGCAGAAACCAGG
GCAGTCTCCTAAACTGCTGATTTACTGGGCATCCACTAGGGAATCTGGGG
TCCCTGATCGCTTCACAGGCAGTGGATCTGGGACAGATTTCACTCTCACC
ATCAGCAGTGTGAAGACTGAAGACCTGGCAGTTTATTACTGTCAGCAATA
TTATAGCTATCGGACGTTCGGTGGAGGCACCAAGCTGGAAATCAAA

(SEQ ID NO: 22; exemplary CDRs are underlined;
see also, Table 19)

MDSQAQVLMSLLLWVSGTCGDIVMSQSPSSLAVSVGEKVTMSCKSSQSLL
YSSNQKNYLAWYQQKPGQSPKLLIYWASTRESGVPDRFTGSGSGTDFTLT
ISSVKTEDLAVYYCQQYYSYRTFGGGTKLEIK

The nucleic acid and amino acid sequences for the heavy chain and light chain variable regions of the antibody R29-7A-54C are shown below:

R29-7A-54C heavy chain variable region

(SEQ ID NO: 23)

ATGGCTGTCCTGGGGCTGCTTCTCTGCCTGGTGACGTTCCCAAGCTGTG
TCCTGTCCCAGGTGCAGCTGAAGGAGTCAGGACCTGGCCTGGTGGCGCCC
TCACAGAGCCTGTCCATCACATGCACTGTCTCAGGGTTCTCATTAACCGA
CTATGCCATAAGCTGGATTCGCCAGCCTCCAGGAAAGGGTCTGGAGTGGC
TGGGAGTAATATGGGGTGGTGGAAGAACATACTCTAATTCAGCTCTCAAA
TCCAGACTGAGCATCAGCAAGGACAACTCCAGGAGCCAAATTTTCTTAAA
AATGAACAGTCTGCAAACTGATGACACAGCCATGTACTACTGTGCCAAAC
ATGAGGAGGTATCCCGTTTTGCTTACTGGGGCCAAGGGACTCTGGTCACT
GTCTCTGTA

(SEQ ID NO: 24; exemplary CDRs are underlined;
see also, Table 25)

MAVLGLLLCLVTFPSCVLSQVQLKESGPGLVAPSQSLSITCTVSGFSLTD
YAISWIRQPPGKGLEWLGVIWGGGRTYSNSALKSRLSISKDNSRSQIFLK
MNSLQTDDTAMYYCAKHEEVSRFAYWGQGTLVTVSV
R29-7A-54C light chain variable region

(SEQ ID NO: 25)

ATGAAGTTGCCTGTTAGGCTGTTGGTGCTGATGTTCTGGATTCCTGCTTC
CATCAGTGATGTTGTGCTGACCCAAACTCCACTCTCCCTGCCTGTCAGTC
TTGGAGATCAAGCCTCCATCTCTTGCAGATCTAGTCAGAGCCTTGTTTAT
AGTAATGGAAACAGCTATTTACATTGGTACCTGCAGAAGCCAGGCCAGTC
TCCAAAGCTCCTGATCTACAAAGTTTCCAACCGATTTTCTGGGGTCCCAG
ACAGGTTCAGTGGCAGTGGATCAGGGACAGATTTCACACTCAAGATCACT
AGAGTGGAGGCTGAGGATCTGGGAGTTTATTTCTGCTCTCAAAGTACACA
TATTCCGCTCACGTTCGGTGCTGGGACCAAGCTGGAGCTGAAA

(SEQ ID NO: 26; exemplary CDRs are underlined;
see also, Table 25)

MKLPVRLLVLMFWIPASISDVVLTQTPLSLPVSLGDQASISCRSSQSLVY
SNGNSYLHWYLQKPGQSPKLLIYKVSNRFSGVPDRFSGSGSGTDFTLKIT
RVEAEDLGVYFCSQSTHIPLTFGAGTKLELK

The nucleic acid and amino acid sequences for the heavy chain and light chain variable regions of the antibody R29-7A-53A are shown below:

R29-7A-53A heavy chain variable region

(SEQ ID NO: 27)

ATGGCTGTCCTGGGGCTGCTTCTCTGCCTGGTGACGTTCCCAAGCTGTG
TCCTGTCCCAGGTGCAGCTGAAGGAGTCAGGACCTGGCCTGGTGGCGCCC
TCACAGAGCCTGTCCATCACATGCACTGTCTCAGGGTTCTCATTAACCGA
CTATGCCATAAGCTGGATTCGCCAGCCTCCAGGAAAGGGTCTGGAGTGGC
TGGGAGTAATATGGGGTGGTGGAAGAACATACTCTAATTCAGCTCTCAAA
TCCAGACTGAGCATCAGCAAGGACAACTCCAGGAGCCAAATTTTCTTAAA
AATGAACAGTCTGCAAACTGATGACACAGCCATGTACTACTGTGCCAAAC
ATGAGGAGGTATCCCGTTTTGCTTACTGGGGCCAAGGGACTCTGGTCACT
GTCTCTGTA

(SEQ ID NO: 28; exemplary CDRs are underlined;
see also, Table 24)

MAVLGLLLCLVTFPSCVLSQVQLKESGPGLVAPSQSLSITCTVSGFSLTD
YAISWIRQPPGKGLEWLGVIWGGGRTYSNSALKSRLSISKDNSRSQIFLK
MNSLQTDDTAMYYCAKHEEVSRFAYWGQGTLVTVSV
R29-7A-53A light chain variable region

(SEQ ID NO: 29)

ATGAAGTTGCCTGTTAGGCTGTTGGTGCTGATGTTCTGGATTCCTGCTTC
CATCAGTGATGTTGTGCTGACCCAAACTCCACTCTCCCTGCCTGTCAGTC
TTGGAGATCAAGCCTCCATCTCTTGCAGATCTAGTCAGAGCCTTGTTTAT
AGTAATGGAAACAGCTATTTACATTGGTACCTGCAGAAGCCAGGCCAGTC
TCCAAAGCTCCTGATCTACAAAGTTTCCAACCGATTTTCTGGGGTCCCAG
ACAGGTTCAGTGGCAGTGGATCAGGGACAGATTTCACACTCAAGATCACT
AGAGTGGAGGCTGAGGATCTGGGAGTTTATTTCTGCTCTCAAAGTACACA
TATTCCGCTCACGTTCGGTGCTGGGACCAAGCTGGAGCTGAAA

(SEQ ID NO: 30; exemplary CDRs are underlined;
see also, Table 24)

MKLPVRLLVLMFWIPASISDVVLTQTPLSLPVSLGDQASISCRSSQSLVY
SNGNSYLHWYLQKPGQSPKLLIYKVSNRFSGVPDRFSGSGSGTDFTLKIT
RVEAEDLGVYFCSQSTHIPLTFGAGTKLELK

The nucleic acid and amino acid sequences for the heavy chain and light chain variable regions of the antibody R29-8-50C are shown below:

R29-8-50C heavy chain variable region

(SEQ ID NO: 31)

ATGAACTTCGGGTTCAGCTTGATTTTCCTTGTCCTTGTTTCAAAAGGTGT
CCAGTGTGAAGTGAAGCTGGTGGAGTCTGGGGGAGGCTTAGTGAAGCCTG
GAGGGTCCCTGAAATTCTCCTGTGCAGCCTCTGGATTCACTTTCAGTAGC
TATGCCGTGTCTTGGGTTCGCCAGACTCCAGAGAAGAGGCTGGAGTGGGT
CGCATCCATTACTAGTGGTGGTAGAACCTACTATCCAGACAGTGTGAAGG
GCCGATTCACCATCTCCAGAGATAATGCCAGGAGCATGTTGTACCTGCAC
ATGAGCAGTCTGAGGTCTGAGGACACGGCCATGTATTACTGTACAAGAGG
CTGGGACGAGAATGACTTATGGGGCCAAGGCACCACTCTCACCGTCTCCT
CA

(SEQ ID NO: 32; exemplary CDRs are underlined;
see also, Table 14)

MNFGFSLIFLVLVSKGVQCEVKLVESGGGLVKPGGSLKFSCAASGFTFSS
YAVSWVRQTPEKRLEWVASITSGGRTYYPDSVKGRFTISRDNARSMLYLH
MSSLRSEDTAMYYCTRGWDENDLWGQGTTLTVSS
R29-8-50C light chain variable region

(SEQ ID NO: 33)

ATGAAGTTGCCTGTTAGGCTGTTGGTGCTGATGTTCTGGATTCCTGCTTC
CAGCAGTGATGTTGTGATGACCCAAACTCCACTCTCCCTGCCTGTCAGTC
TTGGAGATCAAGCCTCCATCTCTTGCAGATCTAGTCAGAGCCTTGTATAC
AGTAATGGAAACACCTATTTACATTGGTACCTGCAGAGGCCAGGCCAGTC
TCCACAGCTCCTGATCTACAAAGTTTCCAACCGATTTTCTGGGGTCCCAG
ACAGGTTCAGTGGCAGTGGATCAGGGACAGATTTCACACTCAAGATCAGC
AGAGTGGAGGCTGAGGATCTGGGAGTTTATTTCTGCTCTCAAACTACACA
TGTTCCGTGGACGTTCGGTGGAGGCACCAAGCTGGAAATCAAA

(SEQ ID NO: 34; exemplary CDRs are underlined;
see also, Table 14)

MKLPVRLLVLMFWIPASSSDVVMTQTPLSLPVSLGDQASISCRSSQSLVY
SNGNTYLHWYLQRPGQSPQLLIYKVSNRFSGVPDRFSGSGSGTDFTLKIS
RVEAEDLGVYFCSQTTHVPWTFGGGTKLEIK

The nucleic acid and amino acid sequences for the heavy chain and light chain variable regions of the antibody R29-8-19B are shown below:

R29-8-19B heavy chain variable region

(SEQ ID NO: 35)

ATGAACTTCGGGTTCAGCTTGATTTTCCTTGTCCTTGTTTCAAAAGGTGT
CCAGTGTGAAGTGAAGCTGGTGGAGTCTGGGGGAGGCTTAGTGAAGCCTG
GAGGGTCCCTGAAATTCTCCTGTGCAGCCTCTGGATTCACTTTCAGTAGC
TATGCCGTGTCTTGGGTTCGCCAGACTCCAGAGAAGAGGCTGGAGTGGGT
CGCATCCATTACTAGTGGTGGTAGAACCTATTATCCAGACAGTGTGAAGG
GCCGATTCACCATCTCCAGAGATAATGCCAGGAGCATGTTGTATTTGCAC
ATGAGCAGTCTGAGGTCTGAGGACACGGCCATGTATTACTGTACAAGAGG
CTGGGACGAGAATGACTTATGGGGCCAAGGCACCACTCTCACCGTCTCCT
CA

(SEQ ID NO: 36; exemplary CDRs are underlined;
see also, Table 13)

MNFGFSLIFLVLVSKGVQCEVKLVESGGGLVKPGGSLKFSCAASGFTFSS
YAVSWVRQTPEKRLEWVASITSGGRTYYPDSVKGRFTISRDNARSMLYLH
MSSLRSEDTAMYYCTRGWDENDLWGQGTTLTVSS
R29-8-19B light chain variable region

(SEQ ID NO: 37)

ATGAAGTTGCCTGTTAGGCTGTTGGTGCTGATGTTCTGGATTCCTGCTTC
CAGAAGTGATGTTGTGATGACCCAAACTCCACTCTCCCTGCCTGTCAGTC
TTGGAGATCAAGCCTCCATCTCTTGCAGATCTAGTCAGAGCCTTGTATAC
AGTAATGGAAACACCTATTTACATTGGTACCTGCAGAAGCCAGGCCAGTC
TCCACAGCTCCTGATCTACAAAGTTTCCAACCGATTTTCTGGGGTCCCAG
ACAGGTTCAGTGGCAGTGGATCAGGGACAGATTTCACACTCAAGATCAGC
AGAGTGGAGGCTGAGGATCTGGGAGTTTATTTCTGCTCTCAAACTACACA
TGTTCCGTGGACGTTCGGTGGAGGCACCAAGCTGGAAATCAAA

(SEQ ID NO: 38; exemplary CDRs are underlined;
see also, Table 13)

MKLPVRLLVLMFWIPASRSDVVMTQTPLSLPVSLGDQASISCRSSQSLVY
SNGNTYLHWYLQKPGQSPQLLIYKVSNRFSGVPDRFSGSGSGTDFTLKIS
RVEAEDLGVYFCSQTTHVPWTFGGGTKLEIK

The nucleic acid and amino acid sequences for the heavy chain and light chain variable regions of the antibody R29-8-58C are shown below:

R29-8-58C heavy chain variable region

(SEQ ID NO: 39)

ATGAACTTCGGGTTCAGCTTGATTTTCCTTGTCCTTGTTTCAAAAGGTGT
CCAGTGTGAAGTGAAGCTGGTGGAGTCTGGGGGAGGCTTAGTGAAGCCTG
GAGGGTCCCTGAAATTCTCCTGTGCAGCCTCTGGATTCACTTTCAGTAGC
TATGCCGTGTCTTGGGTTCGCCAGACTCCAGAGAAGAGGCTGGAGTGGGT
CGCATCCATTACTAGTGGTGGTAGAAGCTACTATCCGGACAGTGTGAAGG
GCCGATTCACCATCTCCAGAGATAATGCTAGGAGCATGTTGTACCTGCAC
ATGAGCAGTCTGAGGTCTGAGGACACGGCCATGTACTACTGTACAAGAGG
CTGGGACGAGAATGACTTATGGGGCCAAGGCACCACTCTCACCGTCTCCT
CA

(SEQ ID NO: 40; exemplary CDRs are underlined;
see also, Table 17)

MNFGFSLIFLVLVSKGVQCEVKLVESGGGLVKPGGSLKFSCAASGFTFSS
YAVSWVRQTPEKRLEWVASITSGGRSYYPDSVKGRFTISRDNARSMLYLH
MSSLRSEDTAMYYCTRGWDENDLWGQGTTLTVSS
R29-8-58C light chain variable region

(SEQ ID NO: 41)

ATGAAGTTGCCTGTTAGGCTGTTGGTGCTGATGTTCTGGATTCCTGCTTC
CAGCAGTGATGTTGTGATGACCCAAACTCCACTCTCCCTGTTTGTCAGTC
TTGGAGATCAAGCCTCCATCTCTTGCAGATCTAGTCAGAGCCTTCTTTAC
AGTAATGGAAACACCTATTTACATTGGTACCTGCAGAGGCCAGGCCAGTC
TCCACAGCTCCTGATCTACAAAGTTTCCAACCGATTTTCTGGGGTCCCAG
ACAGGTTCAGTGGCAGTGGATCAGGGACAGATTTCACACTCAAGATCAGC
AGAGTGGAGGCTGAGGATCTGGGAGTTTATTTCTGCTCTCAAACTACACA
TGTTCCGTGGACGTTCGGTGGAGGCACCAAGCTGGAAATCAAA

(SEQ ID NO: 42; exemplary CDRs are underlined;
see also, Table 17)

MKLPVRLLVLMFWIPASSSDVVMTQTPLSLFVSLGDQASISCRSSQSLLY
SNGNTYLHWYLQRPGQSPQLLIYKVSNRFSGVPDRFSGSGSGTDFTLKIS
RVEAEDLGVYFCSQTTHVPWTFGGGTKLEIK

The nucleic acid and amino acid sequences for the heavy chain and light chain variable regions of the antibody R29-8-9B are shown below:

R29-8-9B heavy chain variable region

(SEQ ID NO: 43)

ATGAACTACGGGTTCAGCTTGATTTTCCTTGTCCTTGTTTTAAAAGGTGT
CCAGTGTGAAGTGAAGCTGGTGGAGTCTGGGGGAGGCGTAGTGAAGCCTG
GAGGGTCCCTGAAACTCTCCTGTGCAGCCTCTGGATTCACTTTCAGTAGC
TATGCCATGTCCTGGTTTCGCCAGACTTCAGAGATGAGTCTGGAGTGGGT
CGCATCCATTACTGGTGGTGGTGGCACCTACTATCCAGACAGTGTGAAGG
GCCGATTCACCATCTCCAGAGATACTGTCAGGAACATCCTGTACCTGCAA
ATGAGAAGTCTGAGGTCTGGGGACACGGCCATATATTACTGTGCAAGGGG
CTGGGACGAGAATGACTACTGGGGCCAAGGCACCACTCTCACAGTCTCCT
CA

(SEQ ID NO: 44; exemplary CDRs are underlined;
see also, Table 6)

MNYGFSLIFLVLVLKGVQCEVKLVESGGGVVKPGGSLKLSCAASGFTFSS
YAMSWFRQTSEMSLEWVASITGGGGTYYPDSVKGRFTISRDTVRNILYLQ
MRSLRSGDTAIYYCARGWDENDYWGQGTTLTVSS
R29-8-9B light chain variable region

(SEQ ID NO: 45)

ATGAAGTTGCCTGTTAGGCTGTTGGTGCTGATGTTCTGGATTCCTGCTTC
CAGAAGTGATGTTGTGATGACCCAAATTCCATTGTCCCTGCCTGTCAGTC
TTGGAGATCAAGCCTCCATCTCTTGCAGATCTAGTCAGAGCCTTGTATTT
AGTAATGGAAACACCTATTTACATTGGTACTTTCAGAAGCCAGGCCAGTC
TCCAAAGCTCCTGATCTACAAAGTTTCCAACCGATTTTCTGGGGTCCCAG
ACAGGTTCAGTGGCAGTGGATCAGGGACAGATTTCATACTCAGAATCAGC
AGAGTGGAGGCTGAGGATCTGGGAGTTTATTTCTGCTCTCAAAGTACACA
TGTTCCGTGGACGTTCGGTGGAGGCACCAAGCTGGAAATCAAA

(SEQ ID NO: 46; exemplary CDRs are underlined;
see also, Table 6)

MKLPVRLLVLMFWIPASRSDVVMTQIPLSLPVSLGDQASISCRSSQSLVF
SNGNTYLHWYFQKPGQSPKLLIYKVSNRFSGVPDRFSGSGSGTDFILRIS
RVEAEDLGVYFCSQSTHVPWTFGGGTKLEIK

The nucleic acid and amino acid sequences for the heavy chain and light chain variable regions of the antibody R29-8-28C are shown below:

R29-8-28C heavy chain variable region

(SEQ ID NO: 47)

ATGAACTTCGGGTTCAGCTTGATTTTCCTTGTCCTTGTTTTAAAAGGTGT
CCAGTCTGAAGTGAAGCTGGTGGAGTCTGGGGGAGGCGTAGTGAAGCCTG
GAGGGTCCCTGACACTCTCCTGTGCAGCCTCTGGATTCACTTTCAGTAAC
TATGCCGTGTCTTGGTTTCGCCAGACTCCGGAGAAGAGGCTGGAGTGGGT
CGCATCCATTACTAGTGGTGGTAGAACCTACTATCCAGACAGTATGAAGG
GCCGATTCACCATCTCCAGAGATAATGCCAGGAACATCTTGTATCTGCAA
ATGAGCAGTCTGAGGTCTGAGGACACGGCCATATATTACTGTGGAAGAGG
CTGGGACGAGAATGACTACTGGGGCCAAGGCACCACTCTCACCGTCTCCT
CA

(SEQ ID NO: 48; exemplary CDRs are underlined;
see also, Table 12)

MNFGFSLIFLVLVLKGVQSEVKLVESGGGVVKPGGSLTLSCAASGFTFSN
YAVSWFRQTPEKRLEWVASITSGGRTYYPDSMKGRFTISRDNARNILYLQ
MSSLRSEDTAIYYCGRGWDENDYWGQGTTLTVSS
R29-8-28C light chain variable region

(SEQ ID NO: 49)

ATGAAGTTGCCTGTTAGGCTGTTGGTGCTGATGTTCTGGATTCCTGCTTC
CTGCAGTGATGTTGTGATGACCCAAACTCCACTCTCCCTGCCTGTCAGTC
TTGGAGATCAAGCCTCCATCTCTTGCAGATCTAGTCAAAGCCTTGTATAC
AGTAATGGAAACACCTATTTACATTGGTACCTGCAGAAGCCAGGCCAGTC
TCCAAAACTCCTGATCTACAAAGTTTCCAACCGATTTTCTGGGGTCCCAG
ACAGGTTCAGTGGCAGTGGATCAGGGACAGATTTCACACTCAAGATCAAC
AGAGTGGAGGCTGAGGATCTGGGAGTTTATTTCTGCTCTCAAACTACACA
TGTTCCGTGGACGTTCGGTGGAGGCACCAAGCTGGAAATCAAA

(SEQ ID NO: 50; exemplary CDRs are underlined;
see also, Table 12)

MKLPVRLLVLMFWIPASCSDVVMTQTPLSLPVSLGDQASISCRSSQSLVY
SNGNTYLHWYLQKPGQSPKLLIYKVSNRFSGVPDRFSGSGSGTDFTLKIN
RVEAEDLGVYFCSQTTHVPWTFGGGTKLEIK

The nucleic acid and amino acid sequences for the heavy chain and light chain variable regions of the antibody R29-8-120B are shown below:

R29-8-120B heavy chain variable region

(SEQ ID NO: 51)

ATGAACTTCGGGTTCAGCTTGATTTTCCTTGTCCTTGTTTTAAAAGGTGT
CCAGTGTAAAGTGAACCTGGTGGAGTCTGGGGGAGGCTTAGTGAAGGCTG
GAGGGTCCCTGAAACTCTCCTGTGCAGCCTCTGGATTCACTTTCAGTATC
TATGCCATGTCTTGGTTTCGCCAGACTCCAGAGAAGAGGCTGGAGTGGGT
CGCATCCATTACTGGTGGTGGTACCAATTTCTATCCAGACAGTGTGAGGG
GCCGATTCACCATCTCCAGAGATAATGTCAGGAACATTCTGTACCTGCAA
ATGAGCAGTCTGAGGTCTGAGGACACGGCCATGTATTACTGTGCAAGAGG
CTGGGACGAGAATGACTATTGGGGCCAAGGCACCACTCTCACAGTCTCCT
CA

(SEQ ID NO: 52; exemplary CDRs are underlined;
see also, Table 10)

MNFGFSLIFLVLVLKGVQCKVNLVESGGGLVKAGGSLKLSCAASGFTFSI
YAMSWFRQTPEKRLEWVASITGGGTNFYPDSVRGRFTISRDNVRNILYLQ
MSSLRSEDTAMYYCARGWDENDYWGQGTTLTVSS
R29-8-120B light chain variable region

(SEQ ID NO: 53)

ATGAAGTTGCCTGTTAGGCTGTTGGTGCTGATGTTCTGGATTCCAGCTTC
CAGCAGTGATGTTGTGATGACCCAAACTCCACTCTCCCTGCCTGTCAGTC
TTGGAGATCAAGCCTCCATCTCTTGCAGATCTAGTCAGAGCCTTGTATAT
AGTAATGGAAACACCTATTTACATTGGTACCTGCAGAAGCCAGGCCAGTC
TCCAAAGCTCCTGATCTACAAAGTTTCCAACCGATTTTCTGGGGTCCCAG
ACAGGTTCAGTGGCAGTGGATCAGGGACAGATTTCACACTCAAGATCAGC
AGAGTGGAGGCTGAGGATCTGGGAGTTTATTTCTGCTCTCAAAGTACACA
TGTTCCGTGGACGTTCGGTGGAGGCACCAAGCTGGAAATCAAA

(SEQ ID NO: 54; exemplary CDRs are underlined;
see also, Table 10)

MKLPVRLLVLMFWIPASSSDVVMTQTPLSLPVSLGDQASISCRSSQSLVY
SNGNTYLHWYLQKPGQSPKLLIYKVSNRFSGVPDRFSGSGSGTDFTLKIS
RVEAEDLGVYFCSQSTHVPWTFGGGTKLEIK

The nucleic acid and amino acid sequences for the heavy chain and light chain variable regions of the antibody R29-8-75B are shown below:

R29-8-75B heavy chain variable region

(SEQ ID NO: 55)

ATGGACTCCAGGCTCAATTTAGTTTTCCTTGTCCTTATTTTAAAAGGTGT
CCAGTGTGATGTGCAGCTGGTGGAGTCTGGGGGAGGCTTAGTGCAGCCTG
GAGGGTCCCGGAAACTCTCCTGTGCAGCCTCTGGATTCACTTTCAGTAGG
TTTGGAATGCACTGGGTTCGTCAGGCTCCAGAGAAGGGGCTGGAGTGGGT
CGCATACATTAGTAGTGGCAGTAGTACCATCTACTATGCAGACACAGTGA
AGGGCCGATTCACCATCTCCAGAGACAATCCCAAGAACACCCTGTTCCTG
CAAATGACCAGTCTAAGGTCTGAGGACACGGCCATGTATTACTGTGCAAG
AGTTGATTACGACGTGGCGCTTGCTTACTGGGGCCAAGGGACTCTGGTCA
CTGTCTCTGCA

(SEQ ID NO: 56; exemplary CDRs are underlined;
see also, Table 18)

MDSRLNLVFLVLILKGVQCDVQLVESGGGLVQPGGSRKLSCAASGFTFSR
FGMHWVRQAPEKGLEWVAYISSGSSTIYYADTVKGRFTISRDNPKNTLFL
QMTSLRSEDTAMYYCARVDYDVALAYWGQGTLVTVSA
R29-8-75B light chain variable region

(SEQ ID NO: 57)

ATGAAGTTGCCTGTTAGGCTGTTGGTGCTGATGTTCTGGATTCCTGCTTC
CAGCAGTGGTGTTTTGATGACCCAAACTCCACTCTCCCTGCCTGTCAGTC
TTGGAGATCAAGCCTCCATCTCTTGCAGATCTAGTCAGAGCATTGTACAT
AGGAATGGAAATACCTATTTAGAATGGTACCTGCAGAAACCAGGCCAGTC
TCCAAAACTCCTGATCTACAAAGTTTCCAACCGATTTTCTGGGGTCCCAG
ACAGGTTCAGTGGCAGTGGATCAGGGACAGATTTCACACTCAAGATCAGC
AGAGTGGAGGCTGAGGATCTGGGAGTTTATTACTGCTTTCAAGGTTCACA
GTGGACGTTCGGTGGAGGCACCAAGCTGGAAATCAAA

(SEQ ID NO: 58; exemplary CDRs are underlined;
see also, Table 18)

MKLPVRLLVLMFWIPASSSGVLMTQTPLSLPVSLGDQASISCRSSQSIVH
RNGNTYLEWYLQKPGQSPKLLIYKVSNRFSGVPDRFSGSGSGTDFTLKIS
RVEAEDLGVYYCFQGSQWTFGGGTKLEIK

The nucleic acid and amino acid sequences for the heavy chain and light chain variable regions of the antibody R29-8-36C are shown below:

R29-8-36C heavy chain variable region

(SEQ ID NO: 59)

ATGAACTTCGGGTTCAGCTTGATTTTCCTTGTCCTTGTTTCAAAAGGTGT
CCAGTGTGAAGTGAAGCTGGTGGAGTCTGGGGGAGGCTTAGTGAAGCCTG
GAGGGTCCCTGAAATTCTCCTGTGCAGCCTCTGGATTCACTTTCAGTAGC
TATGCCGTGTCTTGGGTTCGCCAGACTCCAGAGAAGAGGCTGGAGTGGGT
CGCATCCATTACTAGTGGTGGTAGAAGCTACTATCCGGACAGTGTGAAGG
GCCGATTCACCATCTCCAGAGATAATGCTAGGAGCATGTTGTACCTGCAC
ATGAGCAGTCTGAGGTCTGAGGACACGGCCATGTACTACTGTACAAGAGG
CTGGGACGAGAATGACTTATGGGGCCAAGGCACCACTCTCACCGTCTCCT
CA

(SEQ ID NO: 60; exemplary CDRs are underlined;
see also, Table 16)

MNFGFSLIFLVLVSKGVQCEVKLVESGGGLVKPGGSLKFSCAASGFTFSS
YAVSWVRQTPEKRLEWVASITSGGRSYYPDSVKGRFTISRDNARSMLYLH
MSSLRSEDTAMYYCTRGWDENDLWGQGTTLTVSS
R29-8-36C light chain variable region

(SEQ ID NO: 61)

ATGAAGTTGCCTGTTAGGCTGTTGGTGCTGATGTTCTGGATTCCTGCTTC
CAGCAGTGATGTTGTGATGACCCAAACTCCACTCTCCCTGTTTGTCAGTC
TTGGAGATCAAGCCTCCATCTCTTGCAGATCTAGTCAGAGCCTTCTTTAC
AGTAATGGAAACACCTATTTACATTGGTACCTGCAGAGGCCAGGCCAGTC
TCCACAGCTCCTGATCTACAAAGTTTCCAACCGATTTTCTGGGGTCCCAG
ACAGGTTCAGTGGCAGTGGATCAGGGACAGATTTCACACTCAAGATCAGC
AGAGTGGAGGCTGAGGATCTGGGAGTTTATTTCTGCTCTCAAACTACACA
TGTTCCGTGGACGTTCGGTGGAGGCACCAAGCTGGAAATCAAA

(SEQ ID NO: 62; exemplary CDRs are underlined;
see also, Table 16)

MKLPVRLLVLMFWIPASSSDVVMTQTPLSLFVSLGDQASISCRSSQSLLY
SNGNTYLHWYLQRPGQSPQLLIYKVSNRFSGVPDRFSGSGSGTDFTLKIS
RVEAEDLGVYFCSQTTHVPWTFGGGTKLEIK

The nucleic acid and amino acid sequences for the heavy chain and light chain variable regions of the antibody R29-8-12A are shown below:

R29-8-12A heavy chain variable region

(SEQ ID NO: 63)

ATGAACTTCGGGTTCAGCTTGATTTTCCTTGTCCTTGTTTCAAAAGGTGT
CCAGTGTGAAGTGAAGCTGGTGGAGTCTGGGGGAGGCTTAGTGAAGCCTG
GAGGGTCCCTGAAATTCTCCTGTGCAGCCTCTGGATTCACTTTCAGTAGC
TATGCCGTGTCTTGGGTTCGCCAGACTCCAGAGAAGAGGCTGGAGTGGGT
CGCATCCATTACTAGTGGTGGTAGAACCTATTATCCAGACAGTGTGAAGG
GCCGATTCACCATCTCCAGAGATAATGCCAGGAGCATGTTGTATTTGCAC
ATGAGCAGTCTGAGGTCTGAGGACACGGCCATGTATTACTGTACAAGAGG
CTGGGACGAGAATGACTTATGGGGCCAAGGCACCACTCTCACCGTCTCCT
CA

(SEQ ID NO: 64; exemplary CDRs are
underlined; see also, Table 15)

MNFGFSLIFLVLVSKGVQCEVKLVESGGGLVKPGGSLKFSCAASGFTFSS
YAVSWVRQTPEKRLEWVASITSGGRTYYPDSVKGRFTISRDNARSMLYLH
MSSLRSEDTAMYYCTRGWDENDLWGQGTTLTVSS
R29-8-12A light chain variable region

(SEQ ID NO: 65)

ATGAAGTTGCCTGTTAGGCTGTTGGTGCTGATGTTCTGGATTCCTGCTT
CCAGAAGTGATGTTGTGATGACCCAAACTCCACTCTCCCTGCCTGTCAG
TCTTGGAGATCAAGCCTCCATCTCTTGCAGATCTAGTCAGAGCCTTGTA
TACAGTAATGGAAACACCTATTTACATTGGTACCTGCAGAAGCCAGGCC
AGTCTCCACAGCTCCTGATCTACAAAGTTTCCAACCGATTTTCTGGGGT
CCCAGACAGGTTCAGTGGCAGTGGATCAGGGACAGATTTCACACTCAAG
ATCAGCAGAGTGGAGGCTGAGGATCTGGGAGTTTATTTCTGCTCTCAAA
CTACACATGTTCCGTGGACGTTCGGTGGAGGCACCAAGCTGGAAATCAA
A

(SEQ ID NO: 66; exemplary CDRs are underlined;
see also, Table 15)

MKLPVRLLVLMFWIPASRSDVVMTQTPLSLPVSLGDQASISCRSSQSLV
YSNGNTYLHWYLQKPGQSPQLLIYKVSNRFSGVPDRFSGSGSGTDFTLK
ISRVEAEDLGVYFCSQTTHVPWTFGGGTKLEIK

The nucleic acid and amino acid sequences for the heavy chain and light chain variable regions of the antibody R29-8-93B are shown below:

R29-8-93B heavy chain variable region

(SEQ ID NO: 131)

GAAGTGAAGCTGGTGGAGTCTGGGGGAGGCGTAGTGAAGCCTGGAGGGTC
CCTGAAACTCTCCTGTACAGCCTCTGGATTCACTTTCAGTAGTTATGCCA
TGTCTTGGTTTCGCCAGACTTCAGAGATGAGGCTGGAGTGGGTCGCATCC
ATTACTGGTGGTGGTGGCACCTACTATCCAGACAGTGTGAAGGGCCGATT
CACCATCTCCAGAGATACTGTCAGGAACATCCTATACCTGCAAATGAGAA
GTCTGAGGTCTGGGGACACGGCCATATATTACTGTGCAAGAGGCTGGGAC
GAGAATGACTACTGGGGCCAAGGCACCCCTCTCACAGTCTCCTCA

(SEQ ID NO: 132; exemplary CDRs are underlined;
see also, Table 7)

EVKLVESGGGVVKPGGSLKLSCTASGFTFSSYAMSWFRQTSEMRLEWVAS
ITGGGGTYYPDSVKGRFTISRDTVRNILYLQMRSLRSGDTAIYYCARGWD
ENDYWGQGTPLTVSS

Exemplary light chain variable region sequences suitable for use in conjunction with the R29-8-93B, R29-8-51B, R29-8-30A, R29-8-18B heavy chain variable region sequences shown above for preparation of C16orf54 binding antibodies comprising heavy chain and light chain variable region sequences include: SEQ ID NO:45 or SEQ ID NO:46 (see, e.g., R29-8-9B antibody light chain variable region sequences); SEQ ID NO:53 or SEQ ID NO:54 (see, e.g., R29-8-120B antibody light chain variable region sequences); SEQ ID NO:49 or SEQ ID NO:50 (see, e.g., R29-8-28C antibody light chain variable region sequences); or SEQ ID NO:61 or SEQ ID NO:62 (see, e.g., R29-8-36C antibody light chain variable region sequences).

The nucleic acid and amino acid sequences for the heavy chain and light chain variable regions of the antibody R29-8-51B are shown below:

R29-8-51B heavy chain variable region

(SEQ ID NO: 133)

GAAGTGAAGCTGGTGGAGTCTGGGGGAGACATGGTGAAGCCTGGAGGGTC
CCTGAAACTCTCCTGTGCAGCCTCTGGATTCACTTTCAGTAGCTATGCCA
TGTCTTGGTTTCGCCAGACTTCAGAGATGAGGCTGGAGTGGGTCGCGTCC
ATTACTGGTGGTGGTGGCACCTACTATCCAGACAGTGTGAAGGGCCGATT
CACCATCTCCAGAGATACTGTCAGGAACATCCTGTACCTGCAAATGAGAA
GTCTGAGGTCTGGGGACACGGCCATATATTACTGTGCAAGAGGCTGGGAC
GAGAATGACTACTGGGGCCAAGGCACCACTCTCACCGTCTCCTCA

(SEQ ID NO: 134; exemplary CDRs are underlined;
see also, Table 8)

EVKLVESGGDMVKPGGSLKLSCAASGFTFSSYAMSWFRQTSEMRLEWVAS
ITGGGGTYYPDSVKGRFTISRDTVRNILYLQMRSLRSGDTAIYYCARGWD
ENDYWGQGTTLTVSS

Exemplary light chain variable region sequences suitable for use in conjunction with the R29-8-93B, R29-8-51B, R29-8-30A, R29-8-18B heavy chain variable region sequences shown above for preparation of C16orf54 binding antibodies comprising heavy chain and light chain variable region sequences include: SEQ ID NO:45 or SEQ ID NO:46 (see, e.g., R29-8-9B antibody light chain variable region sequences); SEQ ID NO:53 or SEQ ID NO:54 (see, e.g., R29-8-120B antibody light chain variable region sequences); SEQ ID NO:49 or SEQ ID NO:50 (see, e.g., R29-8-28C antibody light chain variable region sequences); or SEQ ID NO:61 or SEQ ID NO:62 (see, e.g., R29-8-36C antibody light chain variable region sequences).

The nucleic acid and amino acid sequences for the heavy chain and light chain variable regions of the antibody R29-8-30a are shown below:

R29-8-30A heavy chain variable region

(SEQ ID NO: 135)

GAAGTGAAGCTGGTGGAGTCTGGGGGAGGCTTAGTGAAGCCTGGAGGGTC
CCTGAAACTCTCCTGTGCAGCCTCTGGATTCACTTTCAGTAACTATGCCA
TGTCTTGGTTTCGCCAGACTCCAGAGAAGAGGCTGGAGTGGGTCGCATCC
ATTACTGGTGGTGGTAGCACCTACTATCCAGACAGTGTGAAGGGCCGATT
CATCATCTCCAGAGATAATGCCAGGAACATCCTGTACCTGCAAATGAGGA
GTCTGAGGTCTGAGGACACGGCCATGTATTACTGTGCAAGAGGCTGGGAC
GAGAATGACTACTGGGGCCAAGGCACCACTCTCACAGTCTCCTCA

(SEQ ID NO: 136; exemplary CDRs are underlined;
see also, Table 9)

EVKLVESGGGLVKPGGSLKLSCAASGFTFSNYAMSWFRQTPEKRLEWVAS
ITGGGSTYYPDSVKGRFIISRDNARNILYLQMRSLRSEDTAMYYCARGWD
ENDYWGQGTTLTVSS

Exemplary light chain variable region sequences suitable for use in conjunction with the R29-8-93B, R29-8-51B, R29-8-30A, R29-8-18B heavy chain variable region sequences shown above for preparation of C16orf54 binding antibodies comprising heavy chain and light chain variable region sequences include: SEQ ID NO:45 or SEQ ID NO:46 (see, e.g., R29-8-9B antibody light chain variable region sequences); SEQ ID NO:53 or SEQ ID NO:54 (see, e.g., R29-8-120B antibody light chain variable region sequences); SEQ ID NO:49 or SEQ ID NO:50 (see, e.g., R29-8-28C antibody light chain variable region sequences); or SEQ ID NO:61 or SEQ ID NO:62 (see, e.g., R29-8-36C antibody light chain variable region sequences).

The nucleic acid and amino acid sequences for the heavy chain and light chain variable regions of the antibody R29-8-18B are shown below:

R29-8-18B heavy chain variable region

(SEQ ID NO: 137)

GAAGTGAAGCTGGTGGAGTCTGGGGGAGGCTTAGTGAAGCCTGGAGGGTC
CCTGAAACTCTCCTGTGCAGCCTCTGGATTCACTTTCAGTACCTATGCCG
TGTCTTGGTTTCGCCAGACTCCAGAGAGGAGGCTGGAGTGGGTCGCATCC
ATTACTAGTGGTGGTAGCACCTACTATCCAGACAGTGTGAAGGGCCGATT
CACCATCTCCAGAGATAATGCCAGGAACATCCTGTACCTGCAAATGATCA
GTCTGAGGTCTGGGGACACGGCCATGTATTACTGTGCAAGAGGCTGGGAC
GAGAATGACTACTGGGGCCAAGGCACCACTCTCACAGTCTCCTCA

(SEQ ID NO: 134; exemplary CDRs are underlined;
see also, Table 11)

EVKLVESGGGLVKPGGSLKLSCAASGFTFSTYAVSWFRQTPERRLEWVAS
ITSGGSTYYPDSVKGRFTISRDNARNILYLQMISLRSGDTAMYYCARGWD
ENDYWGQGTTLTVSS

Exemplary light chain variable region sequences suitable for use in conjunction with the R29-8-93B, R29-8-51B, R29-8-30A, R29-8-18B heavy chain variable region sequences shown above for preparation of C16orf54 binding antibodies comprising heavy chain and light chain variable region sequences include: SEQ ID NO:45 or SEQ ID NO:46 (see, e.g., R29-8-9B antibody light chain variable region sequences); SEQ ID NO:53 or SEQ ID NO:54 (see, e.g., R29-8-120B antibody light chain variable region sequences); SEQ ID NO:49 or SEQ ID NO:50 (see, e.g., R29-8-28C antibody light chain variable region sequences); or SEQ ID NO:61 or SEQ ID NO:62 (see, e.g., R29-8-36C antibody light chain variable region sequences).

The nucleic acid and amino acid sequences for the heavy chain and light chain variable regions of the antibody R29-7-38C are shown below:

R29-7-38C heavy chain variable region

(SEQ ID NO: 139)

CAGGTGCAGCTGAAGGAGTCAGGACCTGGCCTGGTGGCGCCCTCACAGAG
CCTGTCCATCACATGCACTGTCTCAGGGTTTTCATTAACCGACTATGCCA
TAAGCTGGATTCGCCAGCCTCCAGGAAAGGGTCTGGAGTGGCTGGGAGTA
ATATGGGGTGGTGGAAGAACATATTCTAATTCAGCTCTCAAATCCAGACT
GAGCATCAGCAAGGACAACTCCAGGAGCCAAATTTTCTTAAAAATGAACA
GTCTGCAAACTGATGACACAGCCATGTACTACTGTGCCAAACATGAGGAG
GTATCCCGTTTTGCTCACTGGGGCCAAGGGACTCTGGTCACTGTCTCTGC
A

(SEQ ID NO: 140; exemplary CDRs are underlined;
see also, Table 26)

QVQLKESGPGLVAPSQSLSITCTVSGFSLTDYAISWIRQPPGKGLEWLGV
IWGGGRTYSNSALKSRLSISKDNSRSQIFLKMNSLQTDDTAMYYCAKHEE
VSRFAHWGQGTLVTVSA

Exemplary light chain variable region sequences suitable for use in conjunction with the R29-7-38C, R29-7-49A, R29-7-13A heavy chain variable region sequences shown above for preparation of C16orf54 binding antibodies comprising heavy chain and light chain variable region sequences include: SEQ ID NO:29 or SEQ ID NO:30 (see, e.g., R29-7A-53A antibody light chain variable region sequences).

The nucleic acid and amino acid sequences for the heavy chain and light chain variable regions of the antibody R29-7-49A are shown below:

R29-7-49A heavy chain variable region

(SEQ ID NO: 141)

CAGGTGCAGCTGAAGGAGTCAGGACCTGGCCTGGTGGCGCCCTCACAGAG
CCTGTCCATCACATGCACTGTCTCAGGGTTCTCATTAACCGACTATGGTG
TAAGCTGGATTCGCCAGCCTCCAGGAAAGGGTCTGGAGTGGCTGGGAGTA
ATATGGGGTGGTGGAGGCACATACTATAATTCAGCTCTCAAATCCAGACT
GAGCATCAACAAGGACAACTCCAAGAGCCAAATTTTCTTAAAAATGAACA
GTCTGCAAACTGATGACACAGCCATTTACTACTGTGCCAAACATGAGGAG
GTATCCCGGTTTGCTTACTGGGGCCAAGGGACTCTGGTCACTGTCTCTGC
A

(SEQ ID NO: 142; exemplary CDRs are underlined;
see also, Table 27)

QVQLKESGPGLVAPSQSLSITCTVSGFSLTDYGVSWIRQPPGKGLEWLGV
IWGGGGTYYNSALKSRLSINKDNSKSQIFLKMNSLQTDDTAIYYCAKHEE
VSRFAYWGQGTLVTVSA

Exemplary light chain variable region sequences suitable for use in conjunction with the R29-7-38C, R29-7-49A, R29-7-13A heavy chain variable region sequences shown above for preparation of C16orf54 binding antibodies comprising heavy chain and light chain variable region sequences include: SEQ ID NO:29 or SEQ ID NO:30 (see, e.g., R29-7A-53A antibody light chain variable region sequences).

The nucleic acid and amino acid sequences for the heavy chain and light chain variable regions of the antibody R29-7-13A are shown below:

R29-7-13A heavy chain variable region

(SEQ ID NO: 143)

CAGGTGCAGCTGAAGGAGTCAGGACCTGGCCTGGTGGCGCCCTCACAGAG
CCTGTCCATCACATGCACTGTCTCAGGGTTCTCATTAACCGACTATGCCA
TAAGCTGGATTCGCCAGCCTCCAGGAAAGGGTCTGGAGTGGCTGGGAGTA
ATATGGGGTGGTGGAAGAACATACTCTAATTCAGCTCTCAAATCCAGACT
GAGCATCAGCAAGGACAATTCCAGGAGCCAAATTTTCTTAAAAATGAACA
GTCTGCACACTGAAGACACAGCCGTGTACTACTGTGCCAAACATGAGGAG
GTCTCCCGTTTTGCTTACTGGGGCCAAGGGACTCTGGTCACTGTCTCTGC
A

(SEQ ID NO: 144; exemplary CDRs are underlined;
see also, Table 28)

QVQLKESGPGLVAPSQSLSITCTVSGFSLTDYAISWIRQPPGKGLEWLGV
IWGGGRTYSNSALKSRLSISKDNSRSQIFLKMNSLHTEDTAVYYCAKHEE
VSRFAYWGQGTLVTVSA

Exemplary light chain variable region sequences suitable for use in conjunction with the R29-7-38C, R29-7-49A, R29-7-13A heavy chain variable region sequences shown above for preparation of C16orf54 binding antibodies comprising heavy chain and light chain variable region sequences include: SEQ ID NO:29 or SEQ ID NO:30 (see, e.g., R29-7A-53A antibody light chain variable region sequences).

### Example 4

### Isotyping and Binning of Monoclonal Antibodies

Individual hybridoma supernatants from Example 2 containing antibodies which recognize huC16orf54 were assessed for isotype by ELISA detection using isotype-specific secondary antibodies purchased from Jackson Immunologicals (Goat×IgG1 HRP—Product#115-035-206, Goat×IgG2a HRP—Product#115-035-206, Goat×IgG2b HRP—Product#115-035-207, Goat×IgG3 HRP—Product#115-035-209). Anti-huC16orf54 antibody 7-2A is an IgG2b, whereas 7-1C, 67-4A, and 67-7A are IgG2as.

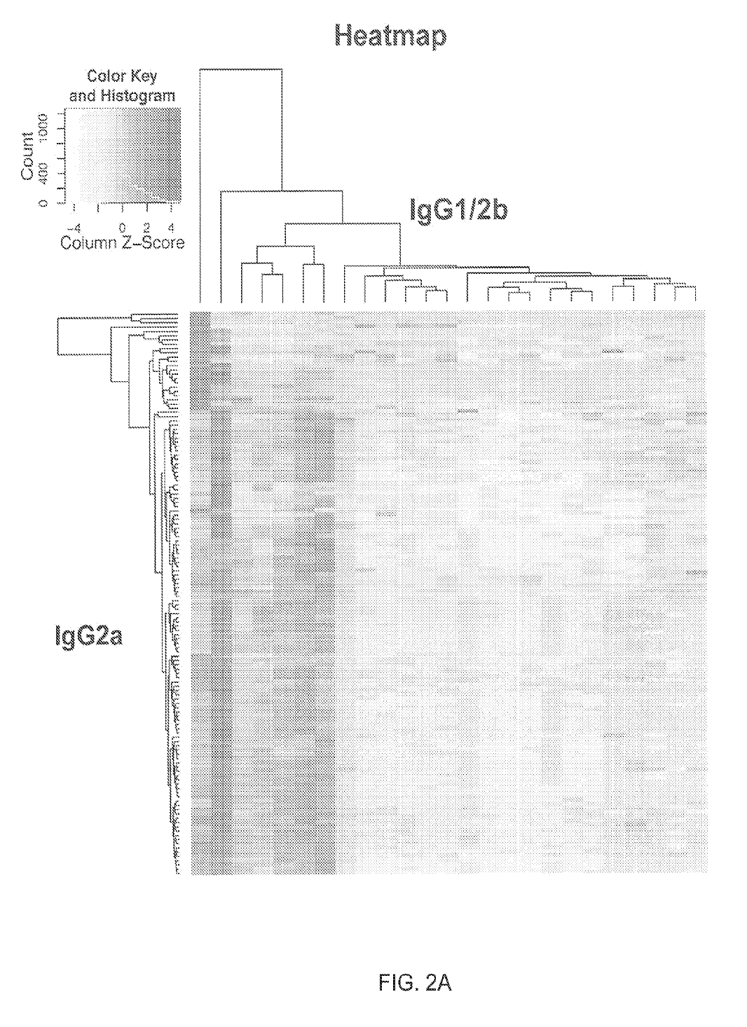

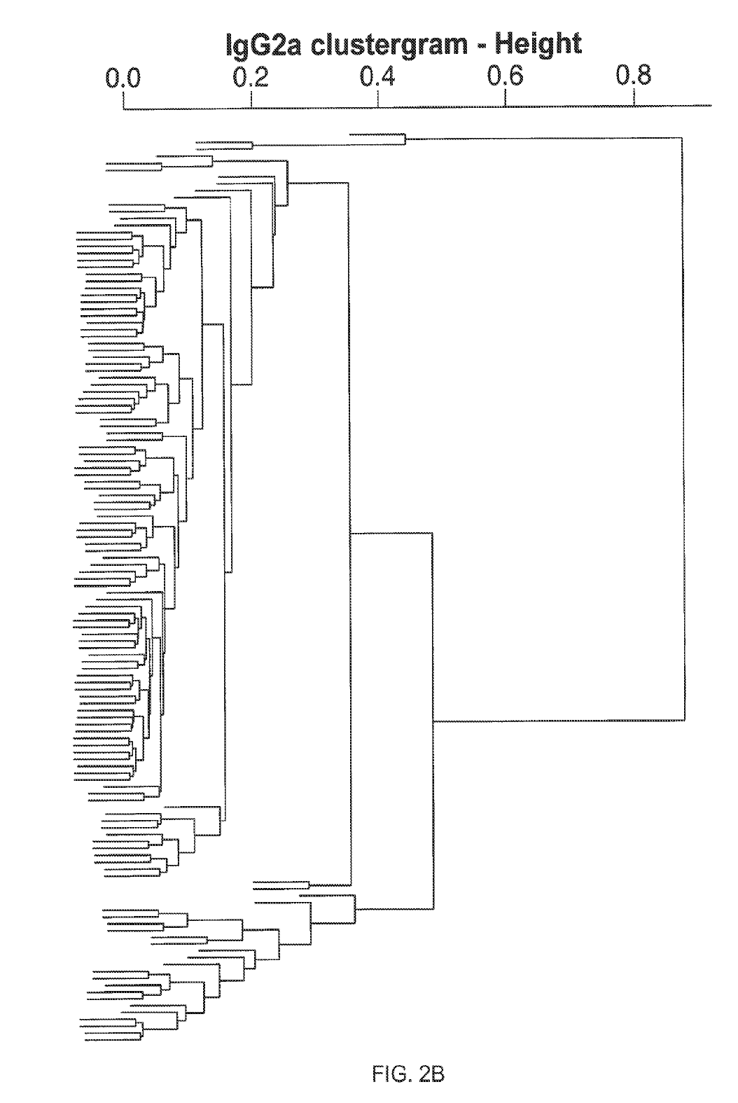

A competition ELISA was performed to establish competitive binding bins. Individual wells containing cells expressing C16orf54 were incubated with either buffer or each anti-huC16orf54 isotyped (e.g., IgG1 b) antibody containing hybridoma supernatant to be used in the competition ELISA. After 1 hour, the wells were washed and fixed using 1% paraformaldehyde. Next, these individual wells of the ELISA plate are incubated for 1 hour with each anti-huC16orf54 isotyped (of a different isotype, e.g., IgG2a) antibody containing hybridoma supernatant. After washing, the wells were incubated with a specific secondary antibody (Jackson Immunologicals Goat×IgG2a HRP—Product#115-035-206) and detected with SuperSignal® ELISA Pico Chemiluminescent substrate (Thermo Scientific—Product#37069). For each anti-huC16orf54 isotyped antibody (of a different isotype, e.g., IgG2a) containing hybridoma supernatant, the luminescence signals from the wells first incubated with each anti-huC16orf54 isotyped (e.g., IgG1) antibody containing hybridoma supernatant are normalized against the luminescence signals from the wells first incubated with buffer only. A heatmap (FIG. 2A) and a clustergram (FIG. 2B) were generated with this normalized data using R (with addition of the R packages gtools, gdata, RColorBrewer and pvclust) for the complete data set. In the heatmap, individual IgG2a isotype antibodies which were able to bind in the presence of an IgG1 are considered to be in a different epitope bin relative to the bin in which the IgG1 resides. Individual IgG2a isotype antibodies which were unable to bind in the presence of an IgG1 are considered to be in the same epitope bin as that particular IgG1. Using this methodology of a competition ELISA as described only one epitope bin was identified for anti-huC16orf54 antibodies, as illustrated in FIG. 2. As confirmed by binding experiments (see Example 5), the C16orf54 antibodies bind to the 32 amino acid N-terminal extracellular domain.

### Example 5

### Binding and Binding Affinity

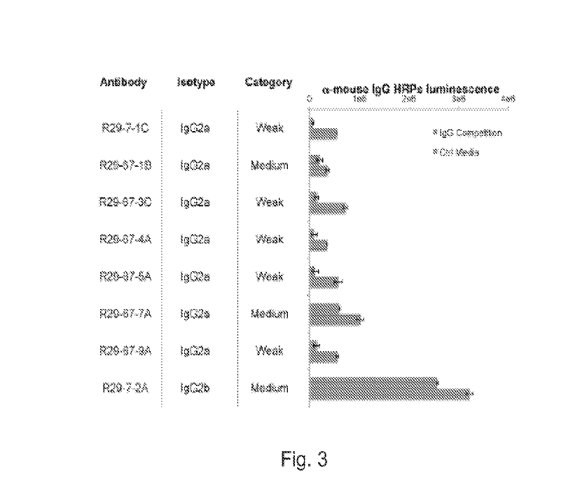

Another competition ELISA was performed to establish relative binding properties. Individual anti-huC16orf54 isotyped (e.g., IgG2a) antibody containing hybridoma supernatants were allowed to bind to huC16orf54 in individual wells of an ELISA plate containing cells expressing C16orf54. After 30 min, the wells were washed. Then individual wells of the ELISA plate were incubated for 2 hours with either buffer or a competing mixture of anti-huC16orf54 isotyped antibodies (of a different isotype, e.g., IgG1 and IgG2b in the case of an IgG2a antibody). After washing, the wells were incubated with a specific secondary antibody (for example, Jackson Immunologicals Goat×IgG2a HRP—Product#115-035-206) and detected with SuperSignal® ELISA Pico Chemiluminescent substrate (Thermo Scientific—Product#37069). For each anti-huC16orf54 isotyped (e.g., IgG2a) antibody containing hybridoma supernatant, relative binding properties were calculated as follows: [(Average luminescence in wells incubated with buffer)−(Average luminescence in wells incubated with mixture of anti-huC16orf54 isotyped antibodies of a different isotype)]/[(Average luminescence in wells incubated with buffer)]. Using this method, it was determined that R29-7-2A undergoes little signal loss upon incubation with the competition mixture, as illustrated in FIG. 3.

Eight anti-huC16orf54 monoclonal antibodies were further screened for binding and subsequent off-rate analysis using the OctetQK384® sytem (ForteBio). Hybridoma supernatants were screened for association to and dissocation from streptavidin sensors coated with the N-terminal 32 amino acids of huC16orf54 coupled to biotin, and the calculated offrates (k_{dis}) are shown in Table 31. Cloned hybridomas with modest to poor off-rates were carried forward for antibody purification.

TABLE 31

| Rank | | | |
|---|---|---|---|
| medium to | | | |
| weak | Antibody ID | kdis (1/s) | kdis Error |
| 1 | R29-67-1B | 5.17 × 10⁻³ | 6.60 × 10⁻⁴ |
| 2 | R29-7-2A | 7.07 × 10⁻³ | 6.06 × 10⁻⁴ |
| 3 | R29-67-4A | 8.06 × 10⁻³ | 1.10 × 10⁻³ |
| 4 | R29-67-7A | 8.45 × 10⁻³ | 6.30 × 10⁻⁴ |
| 5 | R29-67-3C | 9.41 × 10⁻³ | 6.22 × 10⁻⁴ |
| 6 | R29-7-1C | 1.02 × 10⁻² | 8.35 × 10⁻⁴ |
| 7 | R29-67-9A | 1.09 × 10⁻² | 6.83 × 10⁻⁴ |
| 8 | R29-67-5A | 1.22 × 10⁻² | 7.66 × 10⁻⁴ |

Affinity measurements of purified anti-huC16orf54 antibodies were carried out on the OctetQK384® sytem (ForteBio). After coating streptavidin sensors with the N-terminal 32 amino acids of huC16orf54 coupled to biotin at the C-terminus, association and dissociation of purified anti-huC16orf54 antibodies was monitored. KD values were derived using ForteBio's software. The KD values for 7-2A and 7-1C are shown in Table 32.

TABLE 32

| Antibody | kₒₙ (1/Ms) | k_{dis} (1/s) | K_{D} (nM) |
|---|---|---|---|
| R29-7-2A | 31.9 × 10³ ± 4.6 × 10³ | 41.2 × 10⁻⁶ ± 9.7 × 10⁻⁶ | 1.3 ± 0.12 |
| R29-7-1C | 68.1 × 10³ ± 6.2 × 10³ | 79.5 × 10⁻⁶ ± 8.9 × 10⁻⁶ | 1.1 ± 0.03 |

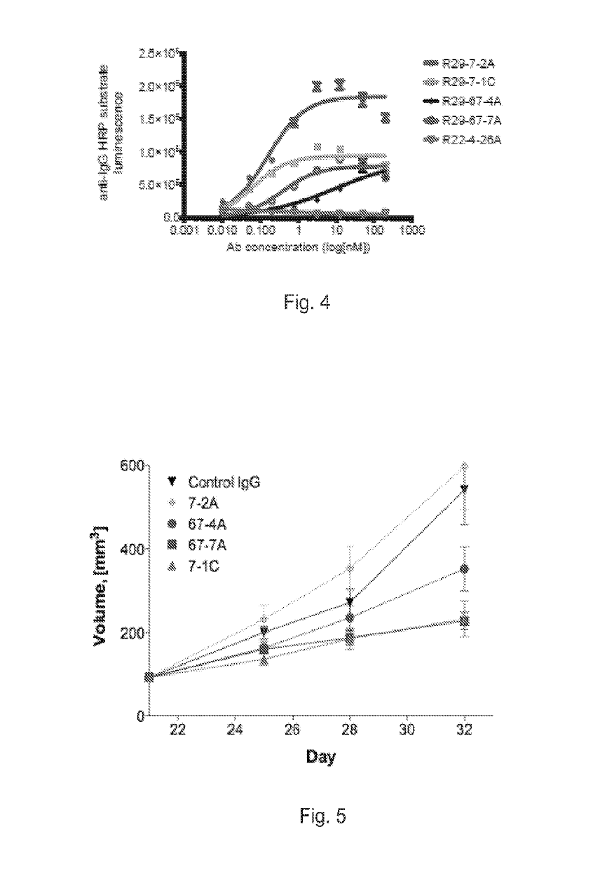

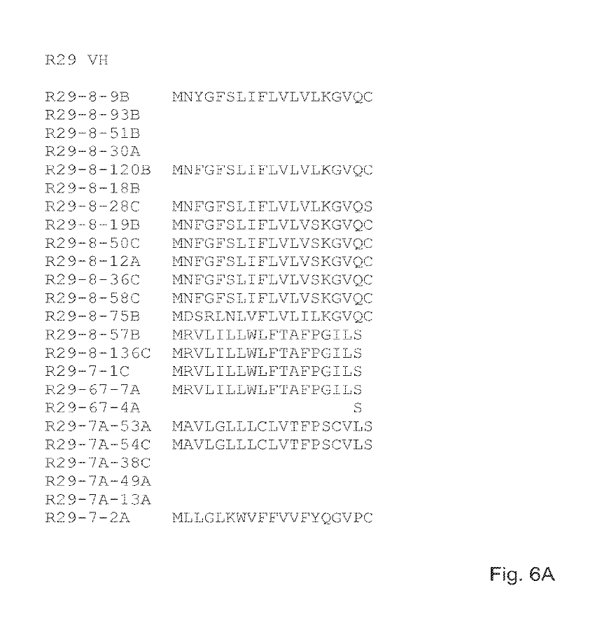

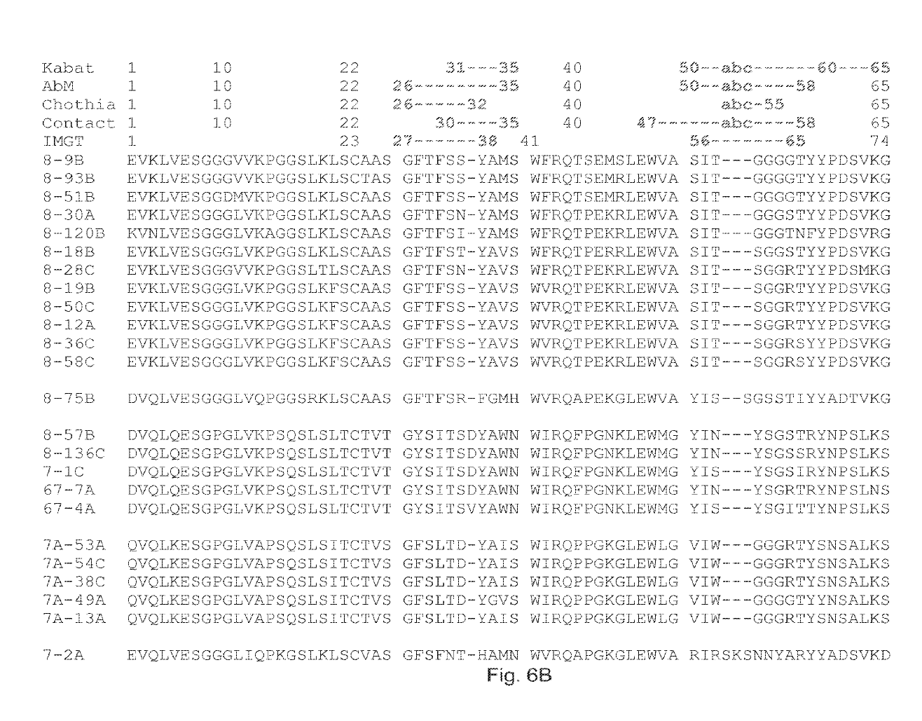

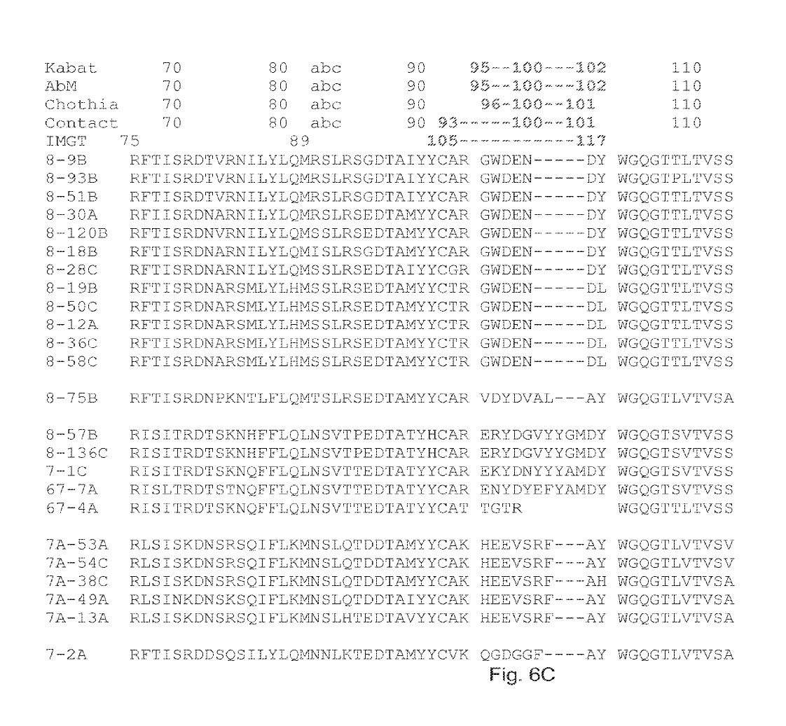

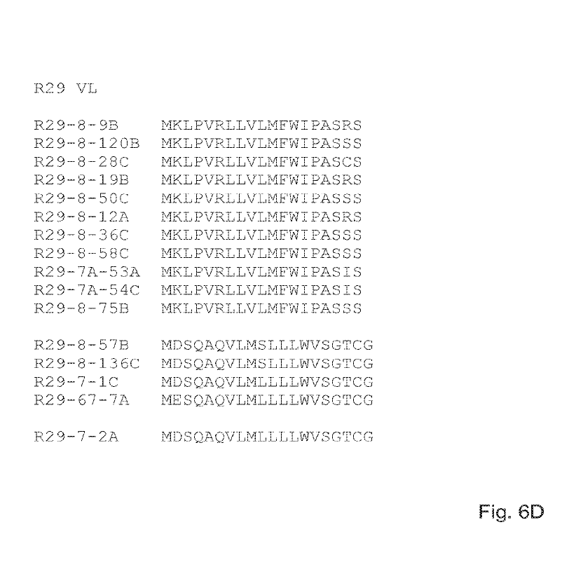

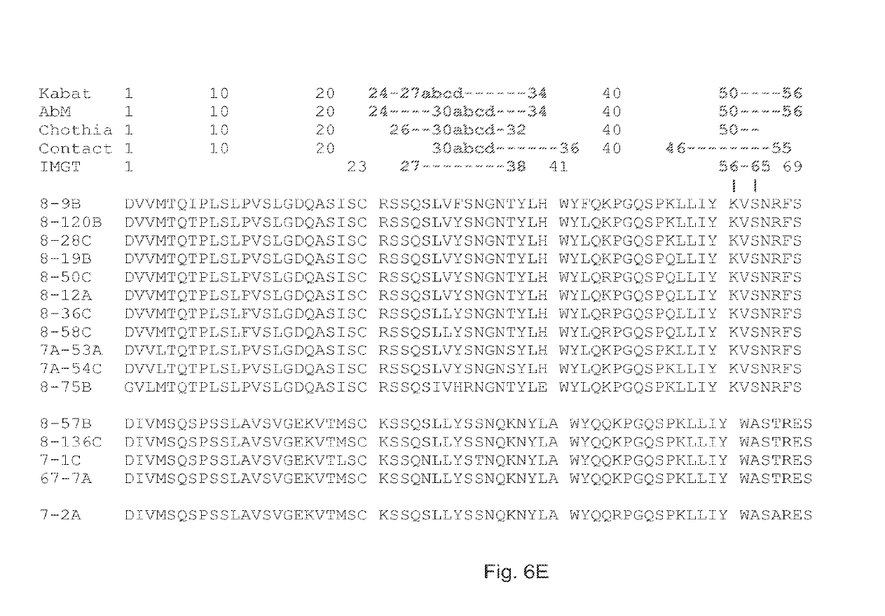

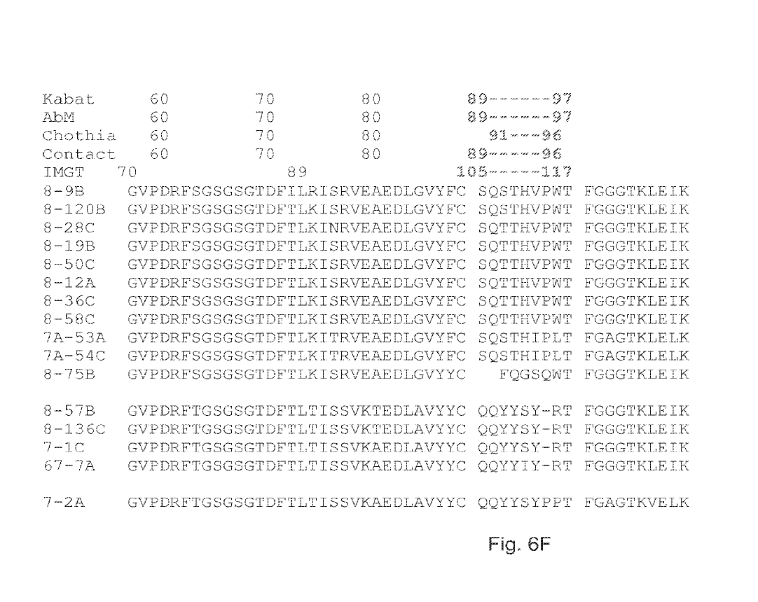

Purified anti-huC16orf54 monoclonal antibodies were also tested for binding by ELISA. huC16orf54-expressing sarcoma cells were incubated with an 1:4 eight-point dilution series starting with 200 nM of anti-huC16orf54 antibody for 2 hours at 4° C. After washing, the cells were incubated with a secondary antibody (Jackson Immunologicals Goat×IgG, Fc specific HRP—Product#115-035-071) and detected with SuperSignal® ELISA Pico Chemiluminescent substrate (Thermo Scientific—Product#37069). EC50 values were determined using Graphpad Prism® software. The titration curves for 7-2A, 7-1C, 67-4A, and 67-7A are shown in FIG. 4. An isotype control, R22-4-26A, is also shown.

Surface expression of huC16orf54 was confirmed in various cell lines (sarcoma cell line, sarcoma cell line expressing hC16orf54, KG-1, HEL9217, REH, WSU-FSCCL, BxPC3, and CFPAC1). After preparing a single cell suspension in PBS supplemented with bovine serum albumin, cells were incubated with 5 μg/ml of huC16orf54 antibody. After two washes, cells were incubated with anti-mouse IgG-PE. After two additional washes, the dead cell indicator TO-PRO3-3 iodide was added to the cell suspension. Upon acquisition of 20,000 events on a BD Biosciences C6 Flow Cytometer, the degree of PE staining was established in live cells using BD C6's analysis software. Using this method, it was shown that huC16orf54 is expressed in the cell lines listed in Table 33. Levels of expression in Table 33 are categorized into four groups: no expression (−), low levels of expression (+ or +/−), medium levels of expression (++), and high levels of expression (+++).

TABLE 33

| Cell line | Cell line origin | C16orf4 staining |
|---|---|---|
| Sarcoma cell line | not applicable | − |
| Sarcoma cell line- | not applicable | +++ |
| C16orf54 | | |
| KG1 | Acute Myelogenous Leukemia | ++ |
| HEL9217 | Erythroleukemia | + |
| REH | Acute Lymphoblastic Leukemia | ++ |
| WSU-FSCCL | B cell lymphoma | ++ |
| BxPCS | Pancreatic Adenocarcinoma | +/− |
| CFPAC1 | Pancreatic Ductal Adenocarcinoma | + |

In additional assays, a variety of tumor cell lines were tested for binding to anti-C16orf54 antibodies and C16orf54 expression using exemplary antibody 7-1C is shown as average mean fluorescence intensity (MFI) in Table 34.

TABLE 34

| | | | Average MFI fold change |
|---|---|---|---|
| | | | relative to isotype control |
| Tumor origin | Cell line | Repeat | (±SEM) |
| Leukemia | KG-1 | 6 | 16.9 ± 1.9 |
| | REH | 3 | 6.4 ± 0.8 |
| | HEL92.1.7 | 3 | 5.0 ± 2.2 |
| | OCI-AML3 | 3 | 3.0 ± 2.0 |
| Lymphoma | WSU-FSCCL | 3 | 13.7 ± 2.8 |
| | KARPAS-1106P | 1 | 10.5 |
| | WSU-DLCL2 | 1 | 9.2 |
| | Granta-519 | 1 | 3.8 |
| Pancreatic | BxPC-3 | 3 | 7.0 ± 2.5 |
| cancer | SW1990 | 3 | 1.9 ± 0.2 |
| | CFPAC-1 | 3 | 1.6 ± 0.0 |
| Colon cancer | NCI-H716 | 2 | 3.4 ± 0.3 |

### Example 6

### Additional Antibody Binding and Characterization

Various assays were performed to establish additional binding properties of anti-C16orf54 antibodies.

### A. Flow Cytometry for Cyno-Crossreactivity Assays

Anti-C16orf54 antibodies which bound to human C16orf54 were tested for their reactivity with human C16orf54 and their crossreactivity with C16orf54 from cynomolgous monkeys (cyno C16orf54) using flow cytometry. Sarcoma cells transfected with and expressing human C16orf54 or cyno C16orf54 were washed with PBS with 0.1% BSA, incubated with FcX receptor blocking solution (BioLegend) for 15 minutes, incubated with 10 μg/ml of anti-C16orf54 antibody for 1 hour at 4° C., washed twice with PBS with 0.1% BSA, incubated with 20 μg/ml of R-Phycoerythrin-AffiniPure F(ab′)2 Fragment Goat Anti-Mouse IgG, Fcγ Fragment Specific (Jackson ImmunoResearch) for 30 minutes at 4° C., washed twice with PBS with 0.1% BSA, incubated with the viability dye TO-PRO-3 iodide (Life Technologies), and immediately analyzed on a MACSQuant Analyzer instrument (Miltenyi). For each cell line and anti-C16orf54 antibody, a fold change was derived by dividing the median fluorescence intensity for Phycoerythrin in the live cell population for the anti-C16orf54 antibody by the median fluorescence intensity for Phycoerythrin in the live cell population for the IgG isotype control antibody. Results are shown in Table 35. A number of anti-C16orf54 antibodies (see, e.g., R29-8-57B, R29-8-136C, R29-7-1C, R29-67-7A, R29-67-4A, and R29-7-2A) bind human C16orf54 and also bind (e.g., are crossreactive with) cyno C16orf54.

TABLE 35

| | | Median fold | Median fold | Flow EC50 on | |
|---|---|---|---|---|---|
| | | change relative to | change relative to | KG1 cells (nM) | Flow EC50 on |
| | | IgG control | IgG control in | [endogenous | F279-cyno- |
| | | in D42M1- | F279-cyno- | expression of | C16orf54 cells |
| # | Antibody | huC16orf54 cells | C16orf54 cells | human C16orf54] | (nM) |
| 1 | R29-8-9B | 194.3 | 2.0 | 1.5 | nd |
| 2 | R29-8-93B | 162.4 | 3.5 | 2.6 | nd |
| 3 | R29-8-51B | 188.2 | 1.7 | 1.3 | nd |
| 4 | R29-8-30A | 107.5 | 1.5 | 0.9 | nd |
| 5 | R29-8-120B | 105.6 | 5.2 | 2.6 | nd |
| 6 | R29-8-18B | 162.4 | 1.7 | 2.5 | nd |
| 7 | R29-8-28C | 156.2 | 3.3 | 2.1 | nd |
| 8 | R29-8-19B | 198.0 | 4.5 | 0.9 | nd |
| 9 | R29-8-50C | 158.7 | 3.2 | 0.7 | nd |
| 10 | R29-8-12A | 193.1 | 3.6 | 1.1 | nd |
| 11 | R29-8-36C | 184.5 | 3.6 | 0.6 | nd |
| 12 | R29-8-58C | 184.5 | 1.4 | 0.6 | nd |
| 13 | R29-8-75B | 148.3 | 7.8 | 6.5 | na |
| 14 | R29-8-57B | 204.0 | 62.0 | 0.1 | 2.5 |
| 15 | R29-8-136C | 201.7 | 61.6 | 0.2 | 2.5 |
| 16 | R29-7-1C | 205.2 | 53.7 | 0.3 | 2.7 |
| 17 | R29-67-7A | 140.0 | 35.6 | 7.3 | 3.7 |
| 18 | R29-67-4A | nd | nd | 20.1 | nd |
| 19 | R29-7A-53A | 5.7 | 1.2 | na | na |
| 20 | R29-7A-54C | 12.1 | 1.0 | na | na |
| 21 | R29-7A-38C | 16.8 | 5.7 | na | na |
| 22 | R29-7A-49A | 58.2 | 0.9 | na | na |
| 23 | R29-7A-13A | 6.0 | 1.4 | na | na |
| 24 | R29-7-2A | 266.9 | 78.5 | 1.0 | 2.7 |
| 25 | R29-67-1B | nd | nd | na | nd |
| 26 | R29-67-3C | nd | nd | na | nd |
| 27 | R29-67-5A | nd | nd | na | nd |
| 28 | R29-67-9A | nd | nd | na | nd |

nd = not done
na = not able to calculate

B. Flow Cytometry for EC50 Assays with Human C16orf54 and Cyno C16orf54 Expressing Cell Lines

Anti-C16orf54 antibodies which bound to human C16orf54 were tested for their binding to cells expressing human C16orf54 and cells expressing cyno C16orf54. Sarcoma cells transfected with and expressing human C16orf54 or cyno C16orf54 were washed with PBS with 0.1% BSA, incubated with FcX receptor blocking solution (BioLegend) for 15 minutes, incubated with 25, 6.25, 1.56, 0.39, 0.098, 0.024, 0.0061 and 0.0015 μg/ml of anti-C16orf54 antibody for 4 hours at 4° C., washed twice with PBS with 0.1% BSA, incubated with 20 μg/ml of R-Phycoerythrin-AffiniPure™ F(ab′)2 Fragment Goat Anti-Mouse IgG, Fcγ Fragment Specific (Jackson ImmunoResearch) for 30 minutes at 4° C., washed twice with PBS with 0.1% BSA, incubated with the viability dye TO-PRO-3 iodide (Life Technologies), and immediately analyzed on a MACSQuant® Analyzer instrument (Miltenyi). Median fluorescence intensities for each primary antibody concentration were used to derive a flow EC50/Kd using the one site-specific binding with Hill slope model in Prism (GraphPad Prism® Software). Results are shown as flow EC50 in Table 35.

C. C16orf54 Peptide Binding Assays.

Anti-C16orf54 antibodies which bind to human C16orf54, including antibodies that are crossreactive with cyno C16orf54, were tested for their binding to various peptides derived from C16orf54 in a peptide ELISA. A 384-well high binding black microplate (Greiner) was incubated with 2 μg/ml of protein (human, cyno, or mouse extracellular domain peptides) for 16 hours, incubated for 1 hr with a blocking buffer (PBS with 5% Fetal Bovine Serum), washed four times with TBS with Tween, incubated for 1 hour with 10 μg/ml of anti-C16orf54 antibody, washed four times with TBS with Tween, incubated for 1 hour with 10 μg/ml of Peroxidase-AffiniPure Goat Anti-Mouse IgG, Fcγ Fragment Specific (Jackson ImmunoResearch), washed four times with TBS with Tween, and peroxidase was detected with a luminometer (Molecular Devices) upon addition of SuperSignal® ELISA Pico Chemiluminescent Substrate (Thermo Scientific). The luminescence signal was determined. Results of binding assays are shown in Table 36.

A number of the anti-C16orf54 antibodies (e.g., R29-8-75B, R29-8-57B, R29-8-136C, R29-7-1C, R29-67-7A and R29-7-2A) bind to the various peptides derived from the extracellular domain (ECD) of C16orf54 and also bind to C16orf54-expressing cells (designated “dual binders”).

TABLE 36

| | | Cyno ECD | Human ECD & ECD | | | |
|---|---|---|---|---|---|---|
| | | bound by | fragments bound by | Peptide | Full kinetics | Full kinetics |
| | Antibody | antibody | antibody | crossblocking | (nM) | (stdev) [#runs] |
| 1 | R29-8-75B | aa1-31 | aa1-31, aa1-15, aa9-24 | aa1-31, aa9-24 | 0.09 | 0.02 [2] |
| 2 | R29-8-57B | | | aa1-31, aa1-15 | 0.60 | 0.12 [3] |
| 3 | R29-8-136C | | | aa1-31, aa1-15 | 0.64 | 0.16 [3] |
| 4 | R29-7-1C | | | none | 0.77 | 0.29 [4] |
| 5 | R29-67-7A | | | aa1-31, aa1-15 | 2.51 | [1] |
| | R29-67-4A | | nd | nd | nd | nd |
| 6 | R29-7-2A | | aa1-31, aa1-15, aa9-24 | aa1-31 | 1.01 | 0.20 [4] |
| | R29-67-1B | | nd | nd | nd | nd |
| | R29-67-3C | | | | | |
| | R29-67-5A | | | | | |
| | R29-67-9A | | | | | |

D. C16orf54 Peptide-Fc Fusion Protein Binding Assays.

Anti-C16orf54 antibodies which bind to human C16orf54, including antibodies that are crossreactive with cyno C16orf54, were tested for their binding to various peptides derived from C16orf54 that were fused to human IgG (e.g., IgG1) constant regions to generate various peptide-Fc fusion proteins in an Fc-based ELISA. The sequences that were used for the various peptide and Fc constructs are shown in Table 37. An exemplary Fc sequence comprises an IgG1 Hinge region sequence and IgG1 CH3 and CH4 domain sequences. As shown in Table 38, a number of peptide-Fc fusion constructs were designed with the extracellular domain of c16orf54 (amino acids 1-31) either N terminal or C-terminal to a human IgG1 Fc domain.

The fusion proteins were transiently expressed in 293 T cells. Protein was purified from the 293 expression supernatants by Protein A-based affinity purification. Exemplary C16 peptide-Fc fusion constructs that were made and purified are shown with their protein yield in Table 38. ELISA assays were performed by coating 384 well plates with the fusion proteins overnight at 4C. Antibodies were analyzed by serial dilution with concentrations ranging from 40 ug/mL to 0.002 mg/mL. Binding was detected with an HRP-conjugated anti mouse secondary antibody. In these assays, a number of the anti-C16orf54 antibodies (e.g., R29-8-75B, R29-8-57B, R29-7-1C, and R29-67-7A) were able to bind to the peptide-Fc fusion proteins and were also dual binders (e.g., able to bind to C16 peptides derived from the C16 ECD and also to C16orf54-expressing cells).

TABLE 37

| Name | Sequences | Length (aa) |
|---|---|---|
| SIT1 peptide | DNCTDLLALGIPSITQ-Lys (Biotin) (SEQ ID | |
| (negative control) | NO: 151) | |
| C16 peptide | MPLTPEPPSGRVEGPPAWEAAPWPSLPCGPC- | |
| (positive control) | C2-Biotin (SEQ ID NO: 152) | |
| Human C16-ECD | MPLTPEPPSGRVEGPPAWEAAPWPSLPCGPC | 31 |
| | (SEQ ID NO: 153) | |
| Cyno C16-ECD | MPSTPEPPSGRMEGPPTWEAAPWPSLPCGPC | 31 |
| | (SEQ ID NO: 154) | |
| 6 His Tag | HHHHHH (SEQ ID NO: 155) | 6 |
| Thrombin site | LVPRGS (SEQ ID NO: 156) | 6 |
| IgG1 Hinge region | DKTHTCPPC (SEQ ID NO: 157) | 9 |
| IgG1 CH3 CH4 | PAPELLGGPSVFLFPPKPKDTLMISRTPEVTCVVV | 218 |
| domains | DVSHEDPEVKFNWYVDGVEVHNAKTKPREEQYN | |
| | STYRVVSVLTVLHQDWLNGKEYKCKVSNKALPAP | |
| | IEKTISKAKGQPREPQVYTLPPSREEMTKNQVSLT | |
| | CLVKGFYPSDIAVEWESNGQPENNYKTTPPVLDS | |
| | DGSFFLYSKLTVDKSRWQQGNVFSCSVMHEALH | |
| | NHYTQKSLSLSPGK (SEQ ID NO: 158) | |
| Mouse kappa | MDFQVQIFSFLLISASVIMSRG (SEQ ID NO: 159) | 22 |
| signal peptide | | |

TABLE 38

| C16-Fc Protein | Yield (mg) |
|---|---|
| KSS-Hinge-Fc-Thr-6His-huC16 ECD | 101 |
| Hu/cynoC16 ECD-6His-Thr-Hinge-Fc | 28 |
| C16 ECD-6His-Thr-Fc | 8 |
| Hu/cynoC16 ECD-6His | nd |

### Example 7

### Binding to C16orf54 Expressing Cells and Copy Number Enumeration

Anti-C16orf54 antibodies were tested for their binding to various cell lines and primary samples (e.g., AML, CLL, lymphoma) using flow cytometry. Copy number of C16orf54 on the various cell lines was determined. Peripheral blood mononuclear (PBMC) and bone marrow mononuclear (BMMC) cells from healthy donors were immunofluorescently stained using the antibodies of CD19, CD3, CD14, CD34, CD33, lineage markers, in addition to an anti-C16orf54 antibody and isotype control antibody, and then evaluated by flow cytometric analysis. Flow results using, for example, monoclonal antibody R29-7-1C, showed low but positive expression of C16orf54 with slightly higher level in BMMCs. In single lineage gated populations, C16orf54 is broadly expressed in CD19+ B cells, CD14+ monocytes, CD34+/lineage—progenitor cells and CD33+ myeloid cells, whereas CD3+ T cells have a distinctively low to undetectable level of C16orf54.

Staining of C16orf54 was observed in AML and lymphoma cells lines, and in primary AML, CLL and lymphoma samples. In some assays, human cell lines and primary samples were immunofluorescently stained using an anti-C16orf54 monoclonal antibody R29-7-1C that was conjugated to AlexaFluor647, and copy number of C16orf54 was determined by interpolation on a calibration curve generated by Quantum™ Simply Cellular® bead standards (Bangs Laboratories, Inc.) Copy number results are shown in Table 39 various cell lines and primary samples expressing C16orf54.

TABLE 39

| | | Bang's analysis |
|---|---|---|
| Type | Name | Average copy # (no. of repeat) |
| Human cell line | KG1/myeloblast | 29,971 (10) |
| | KG1/lymphoblast | 19,975 (1) |
| | KG1A | 11,198 (1) |
| | WSU-FSCCL | 4,221 (3) |
| | WSU-DLCL2 | 5,517 (3) |
| Primary Sample | CLL 1 | 3,979 (1) |
| | CLL 2 | 10,515 (1) |
| | CLL 3 | 12,277 (1) |
| | CLL 4 | 11,240 (1) |
| | MM 1 | 5,798 (1) |
| | MM 2 | 7,590 (1) |
| | Lymphoma 1 | 10,630 (1) |
| | Lymphoma 2 | 34,397 (1) |

In similar experiments with sarcoma cells that were transfected with a C16orf54 gene, average copy number of the expressed C16orf54 was determined to be 406,380 (2 repeats) for C16orf54 expressing sarcoma line 1 and 135,650 (4 repeats) for C16orf54 expressing sarcoma line 2.

### Example 8

### Human C16orf54 Immunohistochemistry (IHC) Analysis

Anti-C16orf54 antibodies were used to detect C16orf54 expression as analyzed by immunohistochemistry (IHC). The transfected cell lines (5×10⁷) and isolated hematological diseased cells were washed in PBS, centrifuged at 300 g for 10 minutes and the resultant cell pellets embedded and frozen in Tissue-Tek OCT compound (commercially available, e.g., Sakura Finetek USA, Inc., Torrence, Calif.; Fisher Scientific USA). 8 μm cryostat sections of the OCT embedded cells and human tissues were post-fixed in a 75% ethanol, 25% acetone mixture for 5 minutes followed by washing in PBS. The slides were then stained with exemplary primary anti-C16orf54 antibodies on a Dako Autostainer using standard avidin-biotin complex (ABC) IHC procedures. In some experiments, the endogenous peroxidase activity was blocked using Bloxall reagent (Vector SP-6000) for 5 minutes followed by avidin/biotin blocking reagents (Vector SP-2001) to block endogenous biotin within tissues. A pre-incubation with a protein blocker (Dako X0909) was performed followed by the primary antibodies incubation for 1 hour at room temperature. In some experiments, primary antibody 7-1C was diluted to a final concentration of 5 μg/ml and primary antibody 67-7A was diluted to a final concentration of 10 μg/ml using antibody diluent (Dako S3022). The control antibody used was a pan-mouse IgG antibody (Life Technologies 08-6599). In some experiments, after washing in PBS, the slides were incubated with a biotinylated horse anti-mouse antibody 1:200 (Vector BA-2001) for 30 minutes followed by a universal streptavidin-peroxidase conjugate (ABC-HRP Vector PK-6100) for 30 minutes. Diaminobenzedine was used as the chromagen (Sigma D5909) and the slides were counterstained with Mayers hematoxylin for 5 minutes, followed by dehydration and coverslipping. Results with the monoclonal anti-C16orf54 antibody 67-7A are shown in Table 40 which confirm the C16orf54 IHC expression in hematological tumors (e.g., AML, CLL, lymphoma).

TABLE 40

| | | C16orf54 | Marker |
|---|---|---|---|
| Indication | Name | (67-7A) | Expression |
| Lymphoma | MD00402 | Positive | CD19+ |
| | OD41411 | Positive | CD19+ |
| | OD41079 | Positive | CD19+ |
| | OD39986 | Positive | CD19+ |
| | WD3517 | Positive | CD19+ |
| CLL | CLL-UF048 | Positive | CD19+ |
| | CLL-UF049 | — | CD19+ |
| | CLL-BC042 | Positive | CD19+ |
| | CLL-BC043 | Positive | CD19+ |
| | CLL-392L | — | CD19+ |
| | CLL-408L | Positive | CD19+ |
| | CLL-413L | — | CD19+ |
| AML | AML-183L | Positive | CD33+ |
| | AML-205L | Positive | CD33+ |
| | AML-211L | Positive | CD33+ |
| | AML-213L | Positive | CD33+ |

### Example 9

### Progenitor Cell Assays

Colony forming unit (CFU) assays were performed to establish if there is binding of anti-C16orf54 antibodies to various progenitor cells including: CFU-E: colony forming unit erythroid (erythroid colony forming cells); BFU-E: burst forming unit erythroid (earliest known erythroid precursor cells); CFU-GM: colony forming unit granulocyte/macrophage (granulocyte-macrophage colony forming cells); and CFU-GEMM: colony forming unit granulocyte, erythrocyte, monocyte/macrophage, megakaryocyte (most primitive colony-forming cells). Normal human bone marrow light density cells derived from normal bone marrow (Lonza, Md.) were stored at −152° C. until utilized for the assays. Clogenic progenitors of the human erythroid and myeloid lineages were set up in methylcellulose-based medium. All test antibodies and reagents were added to the medium at indicated concentrations. Triplicate cultures were initiated for each condition. Following 14 days in culture myeloid and erythroid colonies were assessed microscopically and scored. For these assays, anti-C16orf54 antibodies were evaluated for their direct and indirect effects on human erythroid and myeloid progenitor proliferation. Results using two exemplary antibodies, R29-7-1C and R29-67-7A, are shown in Table 41. When the antibodies were tested at concentrations ranging from 0.032-100 μg/ml they had no effect on colony number or colony size of bone marrow progenitors when added directly into the methylcellulose matrix. Indirect effects on progenitors were assessed by incubating the cells with these antibodies in a liquid culture for 24 hours and subsequent culture of these cells in the methylcellulose matrix. In these indirect assays, the antibodies at concentrations ranging from 0.032-100 μg/ml had no effect on colony number or colony size.

TABLE 41

| | Concentration | | | Total | | | Total |
|---|---|---|---|---|---|---|---|
| Name | (μg/ml) | CFU-E | BFU-E | Erythroid | CFU-GM | CFU-GEMM | CFC |
| Standard | na | 7 +/− 2 | 30 +/− 5 | 38 +/− 3 | 42 +/− 6 | ND | 79 +/− 8 |
| Solvent | na | 8 +/− 2 | 27 +/− 2 | 35 +/− 4 | 45 +/− 4 | ND | 80 +/− 3 |
| 5- | 1 | 4 +/− 2 | ND** | 4 +/− 2^{#} | 1 +/− 1** | ND | 4 +/− 2^{##} |
| Fluorouracil | 0.1 | 6 +/− 2 | 26 +/− 4 | 32 +/− 6 | 26 +/− 3^{$} | ND | 58 +/− 7* |
| | 0.01 | 6 +/− 1 | 31 +/− 1 | 36 +/− 2 | 41 +/− 1 | ND | 77 +/− 2 |
| HB121 | 100 | 7 +/− 2 | 31 +/− 2 | 38 +/− 1 | 41 +/− 6 | ND | 79 +/− 5 |
| (isotype | | | | | | | |
| control) | | | | | | | |
| R29-7- | 100 | 8 +/− 3 | 30 +/− 3 | 38 +/− 4 | 44 +/− 4 | 1 +/− 1 | 82 +/− 4 |
| 1C(anti- | 20 | 8 +/− 2 | 28 +/− 5 | 36 +/− 4 | 40 +/− 5 | 1 +/− 1 | 76 +/− 8 |
| C16orf54) | 4 | 7 +/− 2 | 32 +/− 2 | 38 +/− 3 | 44 +/− 4 | ND | 82 +/− 6 |
| | 0.8 | 8 +/− 2 | 31 +/− 2 | 39 +/− 4 | 42 +/− 4 | 1 +/− 1 | 82 +/− 3 |
| | 0.16 | 9 +/− 2 | 32 +/− 1 | 41 +/− 2 | 44 +/− 6 | ND | 86 +/− 7 |
| | 0.032 | 8 +/− 2 | 31 +/− 2 | 38 +/− 4 | 45 +/− 3 | 1 +/− 1 | 83 +/− 4 |
| R29-67-7A | 100 | 7 +/− 2 | 32 +/− 2 | 39 +/− 3 | 43 +/− 6 | ND | 83 +/− 5 |
| (anti- | 20 | 6 +/− 2 | 33 +/− 3 | 39 +/− 3 | 40 +/− 1 | 1 +/− 1 | 79 +/− 4 |
| C16orf54) | 4 | 9 +/− 2 | 30 +/− 2 | 39 +/− 3 | 43 +/− 2 | ND | 81 +/− 1 |
| | 0.8 | 7 +/− 2 | 32 +/− 3 | 38 +/− 5 | 43 +/− 5 | ND | 81 +/− 6 |
| | 0.16 | 6 +/− 2 | 28 +/− 6 | 35 +/− 7 | 46 +/− 4 | ND | 81 +/− 12 |
| | 0.032 | 7 +/− 2 | 28 +/− 2 | 35 +/− 1 | 39 +/− 3 | ND | 74 +/− 4 |

### Example 10

### Anti-C16orf54 Monoclonal Antibody-Mediated Inhibition of Tumors In Vivo

Antibody efficacy on tumor growth and metastasis formation is studied, e.g., in mouse subcutaneous or orthotopic cancer xenograft models. The antibodies can be unconjugated, or can be conjugated to a therapeutic agent, as appreciated in the art.

Monoclonal antibodies are raised against C16orf54 as described in Example 2 and Example 3, and purified and characterized as described above. Chimeric or humanized antibodies may also be used. A therapeutic monoclonal antibody or a cocktail comprising a mixture of individual monoclonal antibodies is prepared and used for the treatment of mice receiving subcutaneous or orthotopic injections of tumor xenografts.

Subcutaneous tumors are generated by injection of 1×10⁷ cancer cells in a mixture of PBS (without magnesium or calcium) and BD Matrigel™ (BD Biosciences) at a 1:1 ratio in the right flank of female SCID or nu^{-/-} mice. The injected total volume per mouse is 200 ml with 50% being Matrigel (BD Biosciences). Mice are randomized once tumors reach a size between 65-200 mm³. Antibodies are administered weekly, and body weights and tumors are measured once and twice weekly, respectively. Tumor volume is calculated as described (van der Horst et al. (2009) Neoplasia 11: 355-364). As a negative control, mice are injected with either purified mouse IgG or PBS; or a purified monoclonal antibody that recognizes an antigen other than C16orf54.

### Example 11

### Effect of C16orf54 Monoclonal Antibodies on the Growth of Acute Myeloid Lymphoma (AML) Xenografts in Mice

Anti-C16orf54 antibodies were tested for their anti-tumor activity in an animal-tumor model. For these studies, the cell line KG-1 (acute myeloid leukemia) was obtained from ATCC and cultured according to the suppliers' protocols. Animals were obtained from Taconic (Hudson, N.Y.). Studies were conducted with anti-C16orf54 antibodies and antibody-drug conjugates (ADCs) of these antibodies.

In these experiments, 4-6 week-old immunodeficient NOD-SCID female mice were used for the KG-1 tumor model. Mice were subcutaneously injected on the right flank with 6×106 viable cells (KG-1) in a mixture of PBS (without magnesium or calcium) and BD Matrigel™ (BD Biosciences). Once the tumor reached a size between 65-200 mm³ mice were randomized. Anti-C16orf54 antibodies were administered weekly, and bodyweights and tumors were measured once and twice weekly, respectively. Tumor volume was calculated as described (van der Horst et al., supra). Experiments were performed on groups of at least eight animals per experimental point.

Statistical significance between treatment and control groups was calculated using the Graphpad Prism® software package and applying Student's two-tailed t-test. A p-value of less than 0.05 was considered significant.

As shown in FIG. 5, the anti-C16orf54 monoclonal antibodies R29-67-7A and R29-7-1C were inhibitors of tumor growth in the acute myeloid leukemia (KG-1) xenograft as compared to a control IgG. The anti-C16orf54 monoclonal antibody R29-67-4A also inhibited tumor growth in the acute myeloid leukemia (KG-1) xenograft.

In additional experiments, anti-C16orf54 antibodies and ADCs of those antibodies were tested for their in vivo anti-tumor effects in the KG-1 AML model. Results of exemplary experiments shown as tumor growth inhibition (TGI) are shown in Tables 42, 43 and 44.

TABLE 42
C16orf54 Antibodies/ADCs in KG-1 AML Model

| | | | | | p-Value |
|---|---|---|---|---|---|
| | | | | p-Value | (HB121- |
| mAbs | Mean | SD | TGI | (HB121) | MMAF) |
| Control IgG | 3058.6 | 1147.5 | — | — | 0.346 |
| 7-2A | 2572.5 | 841.3 | −17% | 0.352 | 0.990 |
| 7-1C | 2813.4 | 510.5 | −8% | 0.593 | 0.491 |
| 67-7A | 3221.4 | 876.2 | 6% | 0.755 | 0.149 |
| HB-121-mc-MMAF | 2567.0 | 836.3 | −17% | 0.346 | — |
| (control ADC) | | | | | |
| 7-1C-mc-MMAF | 1263.0 | 570.6 | −61% | 0.003 | 0.003 |
| 7-2A-mc-MMAF | 1521.8 | 943.1 | −53% | 0.011 | 0.068 |
| 67-7A-mc-MMAF | 2544.2 | 1252.0 | −18% | 0.406 | 0.967 |

TABLE 43
C16orf54 ADCs in KG-1 AML Model

| mAb | Mean | SD | TGI | p-Value |
|---|---|---|---|---|
| Control IgG-MC-MMAF | 3338.7 | 829.0 | | |
| 8-36C-MC-MMAF | 1040.2 | 1043.6 | −71% | 0.005 |
| 7-1C-VC-MMAE | 1539.4 | 887.0 | −56% | 0.011 |
| 8-57B-MC-MMAF | 1850.2 | 765.4 | −46% | 0.019 |
| 8-19B-MC-MMAF | 1841.9 | 1044.1 | −46% | 0.038 |
| 7-1C-MC-MMAF | 1962.0 | 1132.3 | −42% | 0.063 |
| 8-28C-MC-MMAF | 1777.2 | 1387.5 | −48% | 0.070 |
| 7-2A-VC-MMAE | 2252.0 | 905.5 | −34% | 0.083 |

TABLE 44
C16orf54 ADCs in KG-1 AML Model

| | Dose | | Std | | |
|---|---|---|---|---|---|
| | (mg/ | Volume | Dev | TGI | |
| Treatments | kg) | [mm³] | [mm³] | [%] | p-value |
| CTRL IgG-mc-MMAF | 5 | 2459 | 1025 | — | — |
| 8-36C-mc-MMAF | 5 | 2256 | 902 | −10 | 0.544 |
| 8-36C-mcValCit-MMAE | 5 | 2365 | 923 | −4 | 0.705 |
| 8-57B-mc-MMAF | 5 | 1926 | 420 | −25 | 0.121 |
| 8-57B-mcValCit-MMAE | 5 | 2481 | 867 | 1 | 0.883 |
| 7-1C-mc-MMAF | 5 | 1736 | 600 | −34 | 0.066 |
| 7-1C-mcValCit-MMAF | 5 | 1844 | 506 | −29 | 0.093 |
| 7-1C-mcValCit-MMAE | 5 | 2179 | 615 | −13 | 0.382 |
| 7-1C-mcPEGValCitCC1065 | 5 | 1576 | 834 | −42 | 0.051 |
| Cyclophosphamide | | 1580 | 800 | −42 | 0.048 |
| CTRL IgG-mc-MMAF | 15 | 2131 | 626 | −16 | 0.328 |
| 8-36C-mc-MMAF | 15 | 1926 | 690 | −25 | 0.165 |
| 8-57B-mc-MMAF | 15 | 1485 | 416 | −46 | 0.013 |
| 7-1C-mc-MMAF | 15 | 785 | 270 | −81 | <0.001 |

A number of the ADCs tested had significant affects on tumor growth as measured by tumor growth inhibition (TGI), including when administered at a 15 mg/kg dose as shown in Table 44.

### Example 12

### Preparation and Use of Antibody-Drug Conjugates

Antibody-drug conjugates (ADCs) are prepared and used in secondary ADC assays and direct ADC assays with antibodies to 016orf54 as illustrated in the following generic Scheme A:

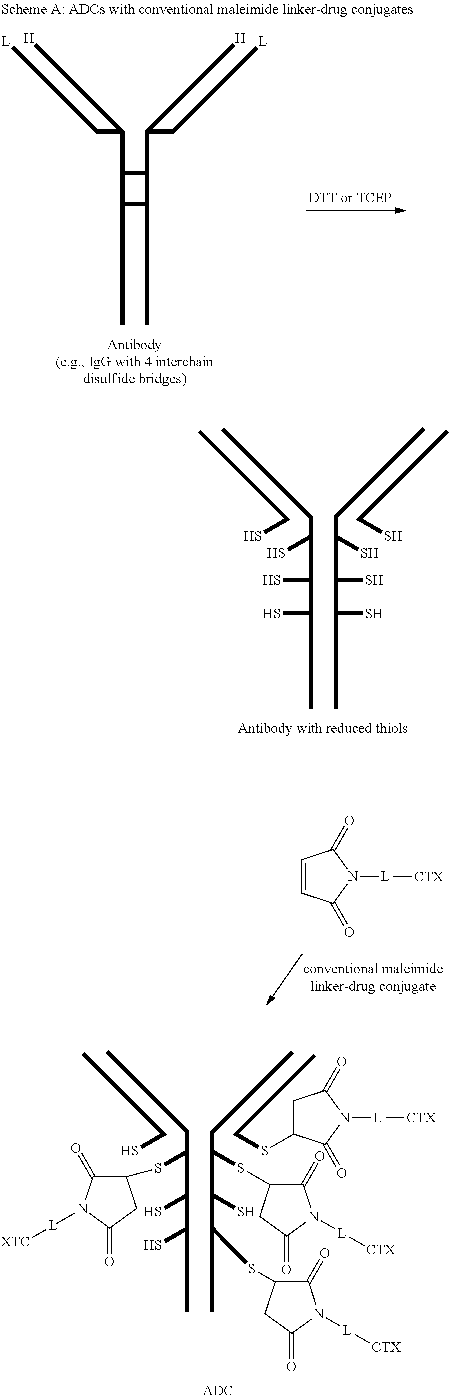

Exemplary antibody-drug conjugates are prepared using maleimidocaproyl-monomethylauristatin F (MC-MMAF), as illustrated in the following Scheme B:

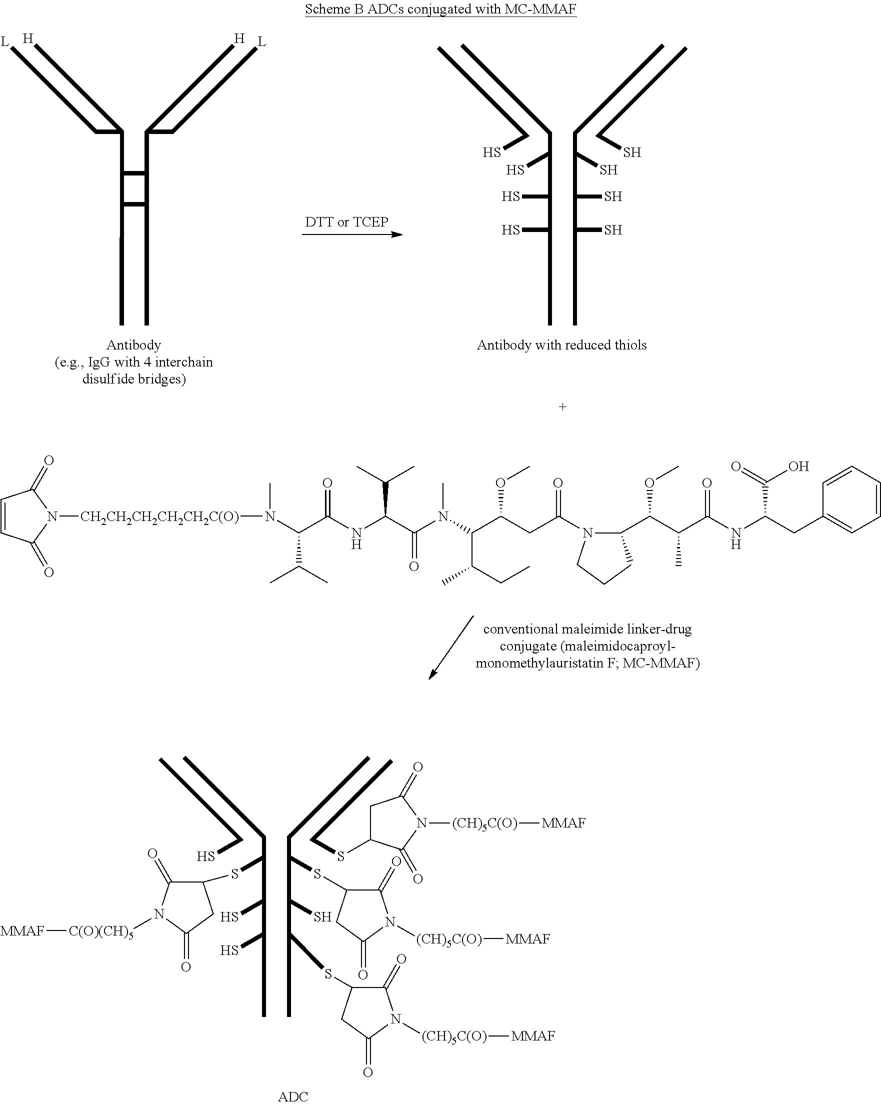

An exemplary synthetic scheme is as follows. In a sterile 1.7 ml eppendorf tube, 20 mg of antibody at 20 mg/ml concentration in phosphate buffered saline (PBS) pH 7.4 (Gibco, Mg and Ca free) is reacted with 1 mM diethylene triamine pentaacetic acid (DTPA) as the chelator. Then 2.75 eq. of tris(2-carboxyethyl)phosphine hydrochloride solution (TCEP HCl) (Sigma ampule 0.5M concentration) or 50 μL of 100 mM dithiothreitol (DTT) is added for an average drug-antibody ratio (DAR) of 4 drugs per antibody and incubated at 37° C. for 1 hour. Dithiobisnitro-benzoate (DTNB; Ellman's reagent) colorimetric assay is used to assess free thiols available for conjugation (Ellman et al., Biochemical Pharmacology 7:88-95 (1961)). The reduced antibody solution is cooled in an ice-bath at ˜0° C. for 15 minutes. Then 60 μL of MC-MMAF from a 10 mM stock solution in DMSO (9.74 mg in 1.074 ml of DMSO for 10 mM) is added and incubated on a roller-plate in a refrigerator at 4° C. overnight (or alternatively at 37° C. for 2 hours). The DTNB assay is repeated to demonstrate no free thiols remaining (clear means no free thiol and a yellow color indicates remaining free thiols and incomplete conjugation of payload). The concentration of the ADC is obtained via the NanoDrop spectrophotometer. The ADC is stored at 4° C.

The anti-tumor activity of unconjugated anti-C16orf54 antibodies and anti-C16orf54 antibodies that were directly conjugated with MMAF (ADCs) was determined in a cell viability assay using several C16orf54-expressing cell lines. Cells were plated at 1000 cells/well and incubated in the presence of antibodies or ADCs for 72 hours. In these in vitro cell-based assays, exemplary MMAF conjugated anti-C16orf54 antibodies (e.g., R29-7-1C-mcMMAF, R29-67-7A-mcMMAF, R29-8-57B-mcMMAF) exhibited killing of the C16orf54-expressing sarcoma cells in a concentration dependent manner: Results are shown as IC50 in Tables 45 and 46. The in vivo activity of the anti-C16orf54 MMAF ADCs was also determined in tumor models as described in Example 11.

TABLE 45
C16orf54 expressing Sarcoma line 1

| Antibody | IC50 (nM) |
|---|---|
| HB121 (isotype control) | n/a |
| R29-7-1C | n/a |
| R29-67-7A | n/a |
| R29-8-57B | n/a |
| MMAE | 0.4 |
| HB121-mcMMAF (control) | n/a |
| R29-7-1C-mcMMAF | 0.1 |
| R29-67-7A mcMMAF | 0.1 |
| R29-8-57B mcMMAF | 0.2 |

TABLE 46
C16orf54 expressing sarcoma line 2

| Antibody | IC50 nM |
|---|---|
| HB121 (isotype control) | n/a |
| R29-7-1C | n/a |
| R29-67-7A | n/a |
| R29-8-57B | n/a |
| MMAE | 4.1 |
| HB121-mcMMAF (control) | n/a |
| R29-7-1C-mcMMAF | 0.2 |
| R29-67-7A mcMMAF | 0.1 |
| R29-8-57B mcMMAF | 0.1 |

### Example 13

### Methods of Synthesizing Additional Anti-C16orf54 Antibody-Drug Conjugates

Alternatively, antibody-drug conjugates of formula (Ia) or (Ib) of the present disclosure may be prepared as illustrated with formula (Ib) in the following Scheme C for:

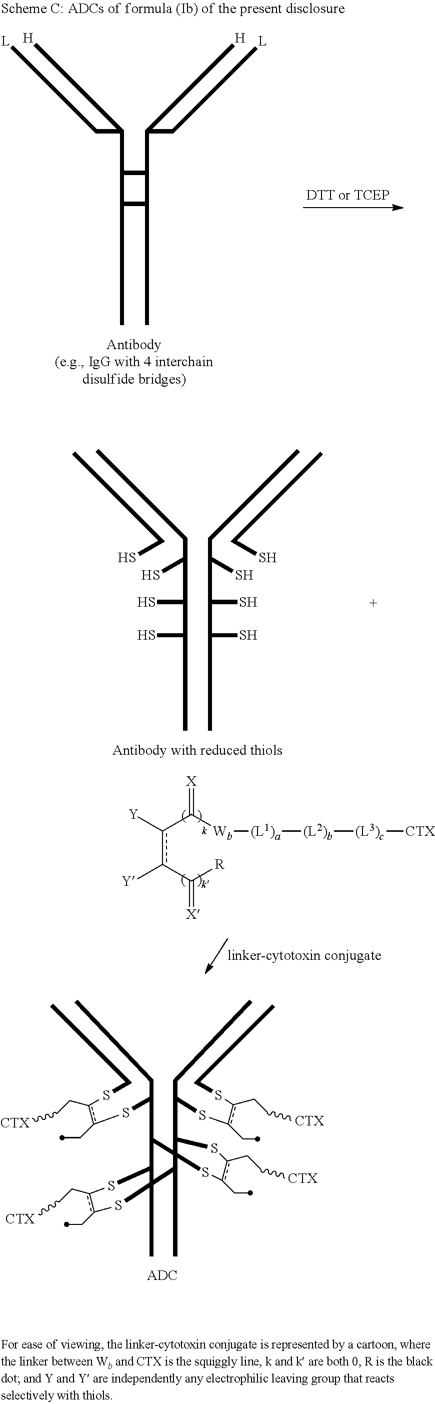

What is claimed:

1. An isolated antibody or functional fragment thereof which specifically binds to the extracellular domain of the polypeptide of SEQ ID NO:1.

2. The antibody or functional fragment of claim 1, wherein the antibody dissociates from the extracellular domain of SEQ ID NO:1 with a K_{d} of 10⁻⁸ M or less as determined by biolayer interferometry.

3. The antibody or functional fragment of claim 1, wherein the antibody dissociates from the extracellular domain of SEQ ID NO:1 with a k_{off} rate constant of 1×10⁻³s⁻¹ or less, as determined by biolayer interferometry.

4. The antibody or functional fragment of claim 1, wherein the antibody dissociates from the extracellular domain of SEQ ID NO:1 with a K_{d} of 10⁻⁸ M or less and a k_{off} rate constant of 1×10⁻³s⁻¹ or less, both determined by biolayer interferometry.

5. The antibody or functional fragment of claim 1, wherein the antibody comprises all three heavy chain complementarity determining regions (CDRs) from a heavy chain variable domain having an amino acid sequence selected from the group consisting of SEQ ID NO:8, SEQ ID NO:4, SEQ ID NO:12, SEQ ID NO:16, SEQ ID NO:20, SEQ ID NO:24, SEQ ID NO:28, SEQ ID NO:32, SEQ ID NO:36, SEQ ID NO:40, SEQ ID NO:44, SEQ ID NO:48, SEQ ID NO:52, SEQ ID NO:56, SEQ ID NO:60, SEQ ID NO:64, SEQ ID NO:132, SEQ ID NO:134, SEQ ID NO:136, SEQ ID NO:138, SEQ ID NO:140, SEQ ID NO:142, SEQ ID NO:144, and SEQ ID NO:146, and/or all three light chain CDRs from a light chain variable domain having an amino acid sequence selected from the group consisting of SEQ ID NO:10, SEQ ID NO:6, SEQ ID NO:14, SEQ ID NO:18, SEQ ID NO:22, SEQ ID NO:26, SEQ ID NO:30, SEQ ID NO:34, SEQ ID NO:38, SEQ ID NO:42, SEQ ID NO:46, SEQ ID NO:50, SEQ ID NO:54, SEQ ID NO:58, SEQ ID NO:62, and SEQ ID NO:66.

6. The antibody or functional fragment of claim 5, wherein the three heavy chain CDRs and/or the three light chain CDRs are determined using IMGT, Kabat, Chothia, Contact or AbM numbering.

7. An isolated antibody or a functional fragment thereof comprising all three heavy chain complementarity determining regions (CDRs) and/or all three light chain CDRs from:
the antibody designated R29-7-1C;
the antibody designated R29-7-2A;
the antibody designated R29-67-7A;
the antibody designated R29-8-136C;
the antibody designated R29-8-57B;
the antibody designated R29-7-54C;
the antibody designated R29-7-53A;
the antibody designated R29-8-50C;
the antibody designated R29-8-19B;
the antibody designated R29-8-58C;
the antibody designated R29-8-9B;
the antibody designated R29-8-28C;
the antibody designated R29-8-120B;
the antibody designated R29-8-75B;
the antibody designated R29-8-36C;
the antibody designated R29-8-12A;
the antibody designated R29-8-93B;
the antibody designated R29-8-51B;
the antibody designated R29-8-30A;
the antibody designated R29-8-18B;
the antibody designated R29-7-38C;
the antibody designated R29-7-49A;
the antibody designated R29-7-13A; or
the antibody designated R29-67-4A.

8. The antibody of claim 7, wherein the antibody or functional fragment thereof comprises all three heavy chain CDRs or all three light chain CDRs from the antibody designated R29-7-2A.

9. The antibody of claim 7, wherein the antibody or functional fragment thereof comprises all three heavy chain CDRs or all three light chain CDRs from the antibody designated R29-7-1C.

10. The antibody of claim 7, wherein the antibody or functional fragment thereof comprises all three heavy chain CDRs or all three light chain CDRs from the antibody designated R29-67-7A.

11. The antibody of claim 7, wherein the antibody or functional fragment thereof comprises all three heavy chain CDRs or all three light chain CDRs from the antibody designated R29-8-136C.

12. The antibody of claim 7, wherein the antibody or functional fragment thereof comprises all three heavy chain CDRs or all three light chain CDRs from the antibody designated R29-8-57B.

13. The antibody of claim 7, wherein the antibody or functional fragment thereof comprises all three heavy chain CDRs or all three light chain CDRs from the antibody designated R29-7-54C.

14. The antibody of claim 7, wherein the antibody or functional fragment thereof comprises all three heavy chain CDRs or all three light chain CDRs from the antibody designated R29-7-53A.

15. The antibody of claim 7, wherein the antibody or functional fragment thereof comprises all three heavy chain CDRs or all three light chain CDRs from the antibody designated R29-8-50C.

16. The antibody of claim 7, wherein the antibody or functional fragment thereof comprises all three heavy chain CDRs or all three light chain CDRs from the antibody designated R29-8-19B.

17. The antibody of claim 7, wherein the antibody or functional fragment thereof comprises all three heavy chain CDRs or all three light chain CDRs from the antibody designated R29-8-58C.

18. The antibody of claim 7, wherein the antibody or functional fragment thereof comprises all three heavy chain CDRs or all three light chain CDRs from the antibody designated R29-8-9B.

19. The antibody of claim 7, wherein the antibody or functional fragment thereof comprises all three heavy chain CDRs or all three light chain CDRs from the antibody designated R29-8-28C.

20. The antibody of claim 7, wherein the antibody or functional fragment thereof comprises all three heavy chain CDRs or all three light chain CDRs from the antibody designated R29-8-120B.

21. The antibody of claim 7, wherein the antibody or functional fragment thereof comprises all three heavy chain CDRs or all three light chain CDRs from the antibody designated R29-8-75B.

22. The antibody of claim 7, wherein the antibody or functional fragment thereof comprises all three heavy chain CDRs or all three light chain CDRs from the antibody designated R29-8-36C.

23. The antibody of claim 7, wherein the antibody or functional fragment thereof comprises all three heavy chain CDRs or all three light chain CDRs from the antibody designated R29-8-12A.

24. The antibody of claim 7, wherein the antibody or functional fragment thereof comprises all three heavy chain CDRs from the antibody designated R29-8-93B.

25. The antibody of claim 7, wherein the antibody or functional fragment thereof comprises all three heavy chain CDRs from the antibody designated R29-8-51B.

26. The antibody of claim 7, wherein the antibody or functional fragment thereof comprises all three heavy chain CDRs from the antibody designated R29-8-30A.

27. The antibody of claim 7, wherein the antibody or functional fragment thereof comprises all three heavy chain CDRs from the antibody designated R29-8-18B.

28. The antibody of claim 7, wherein the antibody or functional fragment thereof comprises all three heavy chain CDRs from the antibody designated R29-7-38C.

29. The antibody of claim 7, wherein the antibody or functional fragment thereof comprises all three heavy chain CDRs from the antibody designated R29-7-49A.

30. The antibody of claim 7, wherein the antibody or functional fragment thereof comprises all three heavy chain CDRs from the antibody designated R29-7-13A.

31. The antibody of claim 7, wherein the antibody or functional fragment thereof comprises all three heavy chain CDRs from the antibody designated R29-67-4A.

32. An isolated antibody or a functional fragment thereof comprising all three heavy chain CDRs from a heavy chain variable domain having an amino acid sequence selected from the group consisting of SEQ ID NO: 8, SEQ ID NO: 4, SEQ ID NO:12, SEQ ID NO:16, SEQ ID NO:20, SEQ ID NO:24, SEQ ID NO:28, SEQ ID NO:32, SEQ ID NO:36, SEQ ID NO:40, SEQ ID NO:44, SEQ ID NO:48, SEQ ID NO:52, SEQ ID NO:56, SEQ ID NO:60, SEQ ID NO:64, SEQ ID NO:132, SEQ ID NO:134, SEQ ID NO:136, SEQ ID NO:138, SEQ ID NO:140, SEQ ID NO:142, SEQ ID NO:144, and SEQ ID NO:146, and all three light chain CDRs from a light chain variable domain having an amino acid sequence selected from the group consisting of SEQ ID NO: 10, SEQ ID NO: 6, SEQ ID NO:14, SEQ ID NO:18, SEQ ID NO:22, SEQ ID NO:26, SEQ ID NO:30, SEQ ID NO:34, SEQ ID NO:38, SEQ ID NO:42, SEQ ID NO:46, SEQ ID NO:50, SEQ ID NO:54, SEQ ID NO:58, SEQ ID NO:62, and SEQ ID NO:66.

33. An isolated antibody or functional fragment thereof comprising all heavy and light chain complementarity determining regions (CDRs) from:
the antibody designated R29-7-2A;
the antibody designated R29-7-1C;
the antibody designated R29-67-7A;
the antibody designated R29-8-136C;
the antibody designated R29-8-57B;
the antibody designated R29-7-54C;
the antibody designated R29-7-53A;
the antibody designated R29-8-50C;
the antibody designated R29-8-19B;
the antibody designated R29-8-58C;
the antibody designated R29-8-9B;
the antibody designated R29-8-28C;
the antibody designated R29-8-120B;
the antibody designated R29-8-75B;
the antibody designated R29-8-36C; or
the antibody designated R29-8-12A.

34. The antibody of claim 33, wherein the antibody or functional fragment thereof comprises all heavy and light chain CDRs from the antibody designated R29-7-2A.

35. The antibody of claim 33, wherein the antibody or functional fragment thereof comprises all heavy and light chain CDRs from the antibody designated R29-7-1C.

36. The antibody of claim 33, wherein the antibody or functional fragment thereof comprises all heavy and light chain CDRs from the antibody designated R29-67-7A.

37. The antibody of claim 33, wherein the antibody or functional fragment thereof comprises all three heavy chain CDRs and all three light chain CDRs from the antibody designated R29-8-136C.

38. The antibody of claim 33, wherein the antibody or functional fragment thereof comprises all three heavy chain CDRs and all three light chain CDRs from the antibody designated R29-8-57B.

39. The antibody of claim 33, wherein the antibody or functional fragment thereof comprises all three heavy chain CDRs and all three light chain CDRs from the antibody designated R29-7-54C.

40. The antibody of claim 33, wherein the antibody or functional fragment thereof comprises all three heavy chain CDRs and all three light chain CDRs from the antibody designated R29-7-53A.

41. The antibody of claim 33, wherein the antibody or functional fragment thereof comprises all three heavy chain CDRs and all three light chain CDRs from the antibody designated R29-8-50C.

42. The antibody of claim 33, wherein the antibody or functional fragment thereof comprises all three heavy chain CDRs and all three light chain CDRs from the antibody designated R29-8-19B.

43. The antibody of claim 33, wherein the antibody or functional fragment thereof comprises all three heavy chain CDRs and all three light chain CDRs from the antibody designated R29-8-58C.

44. The antibody of claim 33, wherein the antibody or functional fragment thereof comprises all three heavy chain CDRs and all three light chain CDRs from the antibody designated R29-8-9B.

45. The antibody of claim 33, wherein the antibody or functional fragment thereof comprises all three heavy chain CDRs and all three light chain CDRs from the antibody designated R29-8-28C.

46. The antibody of claim 33, wherein the antibody or functional fragment thereof comprises all three heavy chain CDRs and all three light chain CDRs from the antibody designated R29-8-120B.

47. The antibody of claim 33, wherein the antibody or functional fragment thereof comprises all three heavy chain CDRs and all three light chain CDRs from the antibody designated R29-8-75B.

48. The antibody of claim 33, wherein the antibody or functional fragment thereof comprises all three heavy chain CDRs and all three light chain CDRs from the antibody designated R29-8-36C.

49. The antibody of claim 33, wherein the antibody or functional fragment thereof comprises all three heavy chain CDRs and all three light chain CDRs from the antibody designated R29-8-12A.

50. The antibody of claim 32, wherein the antibody comprises a heavy chain variable domain sequence selected from the group consisting of SEQ ID NO: 8, SEQ ID NO: 4, SEQ ID NO:12, SEQ ID NO:16, SEQ ID NO:20, SEQ ID NO:24, SEQ ID NO:28, SEQ ID NO:32, SEQ ID NO:36, SEQ ID NO:40, SEQ ID NO:44, SEQ ID NO:48, SEQ ID NO:52, SEQ ID NO:56, SEQ ID NO:60, SEQ ID NO:64, SEQ ID NO:132, SEQ ID NO:134, SEQ ID NO:136, SEQ ID NO:138, SEQ ID NO:140, SEQ ID NO:142, SEQ ID NO:144, and SEQ ID NO:146.

51. The antibody of claim 32, wherein the antibody comprises a light chain variable domain sequence selected from the group consisting of SEQ ID NO: 10, SEQ ID NO: 6, SEQ ID NO:14, SEQ ID NO:18, SEQ ID NO:22, SEQ ID NO:26, SEQ ID NO:30, SEQ ID NO:34, SEQ ID NO:38, SEQ ID NO:42, SEQ ID NO:46, SEQ ID NO:50, SEQ ID NO:54, SEQ ID NO:58, SEQ ID NO:62, and SEQ ID NO:66.

52. Then antibody of claim 50, wherein the antibody further comprises a light chain variable domain sequence selected from the group consisting of SEQ ID NO: 10, SEQ ID NO: 6, SEQ ID NO:14, SEQ ID NO:18, SEQ ID NO:22, SEQ ID NO:26, SEQ ID NO:30, SEQ ID NO:34, SEQ ID NO:38, SEQ ID NO:42, SEQ ID NO:46, SEQ ID NO:50, SEQ ID NO:54, SEQ ID NO:58, SEQ ID NO:62, and SEQ ID NO:66.

53. The antibody of claim 52, wherein the antibody comprises the heavy chain variable domain sequence of SEQ ID NO: 4 and the light chain variable domain sequence of SEQ ID NO: 6.

54. The antibody of claim 52, wherein the antibody comprises the heavy chain variable domain sequence of SEQ ID NO: 8 and the light chain variable domain sequence of SEQ ID NO: 10.

55. The antibody of claim 52, wherein the antibody comprises the heavy chain variable domain sequence of SEQ ID NO: 12 and the light chain variable domain sequence of SEQ ID NO: 14.

56. The antibody of claim 52, wherein the antibody comprises the heavy chain variable domain sequence of SEQ ID NO:16 and the light chain variable domain sequence of SEQ ID NO:18.

57. The antibody of claim 52, wherein the antibody comprises the heavy chain variable domain sequence of SEQ ID NO:20 and the light chain variable domain sequence of SEQ ID NO:22.

58. The antibody of claim 52, wherein the antibody comprises the heavy chain variable domain sequence of SEQ ID NO24: and the light chain variable domain sequence of SEQ ID NO:26.

59. The antibody of claim 52, wherein the antibody comprises the heavy chain variable domain sequence of SEQ ID NO:28 and the light chain variable domain sequence of SEQ ID NO:30.

60. The antibody of claim 52, wherein the antibody comprises the heavy chain variable domain sequence of SEQ ID NO:32 and the light chain variable domain sequence of SEQ ID NO:34.

61. The antibody of claim 52, wherein the antibody comprises the heavy chain variable domain sequence of SEQ ID NO:36 and the light chain variable domain sequence of SEQ ID NO:38.

62. The antibody of claim 52, wherein the antibody comprises the heavy chain variable domain sequence of SEQ ID NO:40 and the light chain variable domain sequence of SEQ ID NO:42.

63. The antibody of claim 52, wherein the antibody comprises the heavy chain variable domain sequence of SEQ ID NO:44 and the light chain variable domain sequence of SEQ ID NO:46.

64. The antibody of claim 52, wherein the antibody comprises the heavy chain variable domain sequence of SEQ ID NO:48 and the light chain variable domain sequence of SEQ ID NO:50.

65. The antibody of claim 52, wherein the antibody comprises the heavy chain variable domain sequence of SEQ ID NO:52 and the light chain variable domain sequence of SEQ ID NO:54.

66. The antibody of claim 52, wherein the antibody comprises the heavy chain variable domain sequence of SEQ ID NO:56 and the light chain variable domain sequence of SEQ ID NO:58.

67. The antibody of claim 52, wherein the antibody comprises the heavy chain variable domain sequence of SEQ ID NO:60 and the light chain variable domain sequence of SEQ ID NO:62.

68. The antibody of claim 52, wherein the antibody comprises the heavy chain variable domain sequence of SEQ ID NO:64 and the light chain variable domain sequence of SEQ ID NO:66.

69. An isolated antibody or functional fragment thereof that binds to C16orf54, wherein the antibody comprises:
(a) a heavy chain variable (V_{H}) region comprising:
(1) a V_{H} CDR1 having an amino acid sequence selected from the group consisting of:
(i) GFTGSX₁YA (SEQ ID NO:67) wherein X₁ is a naturally occurring amino acid,
(ii) GFTFSRFG (SEQ ID NO:73),
(iii) GYSITSX₁YA (SEQ ID NO:79) wherein X₁ is a naturally occurring amino acid,
(iv) GFSLTDYX₁ (SEQ ID NO:85) wherein X₁ is a naturally occurring amino acid, and
(v) GFSFNTHA (SEQ ID NO:91);
(2) a V_{H} CDR2 having an amino acid sequence selected from the group consisting of:
(i) ITGX₁GGX₂X₃ (SEQ ID NO:68) wherein X₁, X₂, and X₃ is a naturally occurring amino acid,
(ii) ISSGSSTI (SEQ ID NO:74),
(iii) IX₁YSGX₂X₃ (SEQ ID NO:80) wherein X₁, X₂, and X₃ is a naturally occurring amino acid,
(iv) IWGGGX₁T (SEQ ID NO:86) wherein X₁ is a naturally occurring amino acid, and
(v) IRSKSNNYAR (SEQ ID NO:92); and
(3) a V_{H} CDR3 having an amino acid sequence selected from the group consisting of:
(i) X₁RGWDENDX₂(SEQ ID NO:69) wherein X₁ and X₂ is a naturally occurring amino acid,
(ii) ARVDYDVALAY (SEQ ID NO:75),
(iii) AREX₁YDX₂X₃X₄YX₅MDY (SEQ ID NO:81) wherein X₁, X₂, X₃, X₄, and X₅ is a naturally occurring amino acid, or alternatively ATTGTR (SEQ ID NO:121),
(iv) AKHEEVSRFAX₁ (SEQ ID NO:87) wherein X₁ is a naturally occurring amino acid, and
(v) VKQGDGGFAY (SEQ ID NO:93);
and/or
(b) a light chain variable (V_{L}) region comprising:
(1) a V_{L} CDR1 having an amino acid sequence selected from the group consisting of:
(i) QSLX₁X₂SNGNTY (SEQ ID NO:70) wherein X₁ and X₂ is a naturally occurring amino acid,
(ii) QSIVHRNGNTY (SEQ ID NO:76),
(iii) QX₁LLYSX₂NQKNY (SEQ ID NO:82) wherein X₁ and X₂ is a naturally occurring amino acid,
(iv) QSLVYSNGNSY (SEQ ID NO:88), and
(v) QSLLYSSNQKNY (SEQ ID NO:94);
(2) a V_{L} CDR2 having an amino acid sequence of:
(i) KVS (SEQ ID NO:71), or
(ii) WAS (SEQ ID NO:83); and
(3) a V_{L} CDR3 having an amino acid sequence selected from the group consisting of:
(i) SQX₁THVPWT (SEQ ID NO:72) wherein X₁ is a naturally occurring amino acid,
(ii) FQGSQWT (SEQ ID NO:78),
(iii) QQYYX₁YRT (SEQ ID NO:84) wherein X₁ is a naturally occurring amino acid,
(iv) SQSTHIPLT (SEQ ID NO:90), and
(v) QQYYSYPPT (SEQ ID NO:96).

70. The antibody of claim 69, wherein the antibody comprises a heavy chain variable (V_{H}) region comprising:
(1) a V_{H} CDR1 having an amino acid sequence selected from the group consisting of:
(i) GFTGSX₁YA (SEQ ID NO:67) wherein X₁ is a naturally occurring amino acid,
(ii) GFTFSRFG (SEQ ID NO:73),
(iii) GYSITSX₁YA (SEQ ID NO:79) wherein X₁ is a naturally occurring amino acid,
(iv) GFSLTDYX₁ (SEQ ID NO:85) wherein X₁ is a naturally occurring amino acid, and
(v) GFSFNTHA (SEQ ID NO:91);
(2) a V_{H} CDR2 having an amino acid sequence selected from the group consisting of:
(i) ITGX₁GGX₂X₃ (SEQ ID NO:68) wherein X₁, X₂, and X₃ is a naturally occurring amino acid,
(ii) ISSGSSTI (SEQ ID NO:74),
(iii) IX₁YSGX₂X₃ (SEQ ID NO:80) wherein X₁, X₂, and X₃ is a naturally occurring amino acid,
(iv) IWGGGX₁T (SEQ ID NO:86) wherein X₁ is a naturally occurring amino acid, and
(v) IRSKSNNYAR (SEQ ID NO:92); and
(3) a V_{H} CDR3 having an amino acid sequence selected from the group consisting of:
(i) X₁RGWDENDX₂(SEQ ID NO:69) wherein X₁ and X₂ is a naturally occurring amino acid,
(ii) ARVDYDVALAY (SEQ ID NO:75),
(iii) AREX₁YDX₂X₃X₄YX₅MDY (SEQ ID NO:81) wherein X₁, X₂, X₃, X₄, and X₅ is a naturally occurring amino acid, or alternatively ATTGTR (SEQ ID NO:121),
(iv) AKHEEVSRFAX₁ (SEQ ID NO:87) wherein X₁ is a naturally occurring amino acid, and
(v) VKQGDGGFAY (SEQ ID NO:93).

71. The antibody of claim 69, wherein the antibody comprises a light chain variable (V_{L}) region comprising:
(1) a V_{L} CDR1 having an amino acid sequence selected from the group consisting of:
(i) QSLX₁X₂SNGNTY (SEQ ID NO:70) wherein X₁ and X₂ is a naturally occurring amino acid,
(ii) QSIVHRNGNTY (SEQ ID NO:76),
(iii) QX₁LLYSX₂NQKNY (SEQ ID NO:82) wherein X₁ and X₂ is a naturally occurring amino acid,
(iv) QSLVYSNGNSY (SEQ ID NO:88), and
(v) QSLLYSSNQKNY (SEQ ID NO:94);
(2) a V_{L} CDR2 having an amino acid sequence of:
(i) KVS (SEQ ID NO:71), or
(ii) WAS (SEQ ID NO:83); and
(3) a V_{L} CDR3 having an amino acid sequence selected from the group consisting of:
(i) SQX₁THVPWT (SEQ ID NO:72) wherein X₁ is a naturally occurring amino acid,
(ii) FQGSQWT (SEQ ID NO:78),
(iii) QQYYX₁YRT (SEQ ID NO:84) wherein X₁ is a naturally occurring amino acid,
(iv) SQSTHIPLT (SEQ ID NO:90), and
(v) QQYYSYPPT (SEQ ID NO:96).

72. The antibody of claim 69, wherein the antibody comprises:
(a) a heavy chain variable (V_{H}) region comprising:
(1) a V_{H} CDR1 having an amino acid sequence of GFTGSX₁YA (SEQ ID NO:67) wherein X₁ is a naturally occurring amino acid;
(2) a V_{H} CDR2 having an amino acid sequence of ITGX₁GGX₂X₃ (SEQ ID NO:68) wherein X₁, X₂, and X₃ is a naturally occurring amino acid; and
(3) a V_{H} CDR3 having an amino acid sequence of X₁RGWDENDX₂ (SEQ ID NO:69) wherein X₁ and X₂ is a naturally occurring amino acid; and
(b) a light chain variable (V_{L}) region comprising:
(1) a V_{L} CDR1 having an amino acid sequence of QSLX₁X₂SNGNTY (SEQ ID NO:70) wherein X₁ and X₂ is a naturally occurring amino acid;
(2) a V_{L} CDR2 having an amino acid sequence of KVS (SEQ ID NO:71); and
(3) a V_{L} CDR3 having an amino acid sequence of SQX₁THVPWT (SEQ ID NO:72) wherein X₁ is a naturally occurring amino acid.

73. The antibody of claim 72, wherein the antibody comprises:
(a) a heavy chain variable (V_{H}) region comprising:
(1) a V_{H} CDR1 having an amino acid sequence of GFTGSX₁YA (SEQ ID NO:67) wherein X₁ is S, N, I or T;
(2) a V_{H} CDR2 having an amino acid sequence of ITGX₁GGX₂X₃ (SEQ ID NO:68) wherein X1 is G or S, wherein X2 is G, S, T or R, wherein X3 is T, N or S; and
(3) a V_{H} CDR3 having an amino acid sequence of X₁RGWDENDX₂ (SEQ ID NO:69) wherein X1 is A, G or T, wherein X2 is Y or L; and
(b) a light chain variable (V_{L}) region comprising:
(1) a V_{L} CDR1 having an amino acid sequence of QSLX₁X₂SNGNTY (SEQ ID NO:70) wherein X1 is V or L, wherein X2 is F or Y;
(2) a V_{L} CDR2 having an amino acid sequence of KVS (SEQ ID NO:71); and
(3) a V_{L} CDR3 having an amino acid sequence of SQX₁THVPWT (SEQ ID NO:72) wherein X1 is S or T.

74. The antibody of claim 73, wherein the antibody comprises:
(a) a heavy chain variable (V_{H}) region comprising:
(1) a V_{H} CDR1 having an amino acid sequence selected from the group consisting of SEQ ID NO:76, SEQ ID NO:89, SEQ ID NO:95, and SEQ ID NO:96;
(2) a V_{H} CDR2 having an amino acid sequence selected from the group consisting of SEQ ID NO:97, SEQ ID NO:98, SEQ ID NO:99, SEQ ID NO:100, SEQ ID NO:101, and SEQ ID NO:102; and
(3) a V_{H} CDR3 having an amino acid sequence selected from the group consisting of SEQ ID NO:103, SEQ ID NO:104, and SEQ ID NO:105; and
(b) a light chain variable (V_{L}) region comprising:
(1) a V_{L} CDR1 having an amino acid sequence selected from the group consisting of SEQ ID NO:106, SEQ ID NO:107, and SEQ ID NO:108;
(2) a V_{L} CDR2 having an amino acid sequence of KVS (SEQ ID NO:71); and
(3) a V_{L} CDR3 having an amino acid sequence of SEQ ID NO:109 or SEQ ID NO:110.

75. The antibody of claim 69, wherein the antibody comprises:
(a) a heavy chain variable (V_{H}) region comprising:
(1) a V_{H} CDR1 having an amino acid sequence of GFTFSRFG (SEQ ID NO:73);
(2) a V_{H} CDR2 having an amino acid sequence of ISSGSSTI (SEQ ID NO:74); and
(3) a V_{H} CDR3 having an amino acid sequence of ARVDYDVALAY (SEQ ID NO:75); and
(b) a light chain variable (V_{L}) region comprising:
(1) a V_{L} CDR1 having an amino acid sequence of QSIVHRNGNTY (SEQ ID NO:76);
(2) a V_{L} CDR2 having an amino acid sequence of KVS (SEQ ID NO:71); and
(3) a V_{L} CDR3 having an amino acid sequence of FQGSQWT (SEQ ID NO:78).

76. The antibody of claim 69, wherein the antibody comprises:
(a) a heavy chain variable (V_{H}) region comprising:
(1) a V_{H} CDR1 having an amino acid sequence of GYSITSX₁YA (SEQ ID NO:79) wherein X₁ is a naturally occurring amino acid;
(2) a V_{H} CDR2 having an amino acid sequence of IX₁YSGX₂X₃ (SEQ ID NO:80) wherein X₁, X₂, and X₃ is a naturally occurring amino acid; and
(3) a V_{H} CDR3 having an amino acid sequence of AREX₁YDX₂X₃X₄YX₅MDY (SEQ ID NO:81) wherein X₁, X₂, X₃, X₄, and X₅ is a naturally occurring amino acid, or alternatively ATTGTR (SEQ ID NO:121);
and
(b) a light chain variable (V_{L}) region comprising:
(1) a V_{L} CDR1 having an amino acid sequence of QX₁LLYSX₂NQKNY (SEQ ID NO:82) wherein X₁ and X₂ is a naturally occurring amino acid;
(2) a V_{L} CDR2 having an amino acid sequence of WAS (SEQ ID NO:83); and
(3) a V_{L} CDR3 having an amino acid sequence of QQYYX₁YRT (SEQ ID NO:84) wherein X₁ is a naturally occurring amino acid.

77. The antibody of claim 76, wherein the antibody comprises:
(a) a heavy chain variable (V_{H}) region comprising:
(1) a V_{H} CDR1 having an amino acid sequence of GYSITSX₁YA (SEQ ID NO:79) wherein X1 is D or V;
(2) a V_{H} CDR2 having an amino acid sequence of IX₁YSGX₂X₃ (SEQ ID NO:80) wherein X1 is N or S, wherein X2 is S, R or I, wherein X3 is T, S, I; and
(3) a V_{H} CDR3 having an amino acid sequence of AREX₁YDX₂X₃X₄YX₅MDY (SEQ ID NO:81) wherein X1 is R, K or N, wherein X2 is G, N or Y, wherein X3 is V, Y or E, wherein X4 is Y or F, X5 is G or A, or alternatively ATTGTR (SEQ ID NO:121);
and
(b) a light chain variable (V_{L}) region comprising:
(1) a V_{L} CDR1 having an amino acid sequence of QX₁LLYSX₂NQKNY (SEQ ID NO:82) wherein X1 is S or N, wherein X2 is S or T;
(2) a V_{L} CDR2 having an amino acid sequence of WAS (SEQ ID NO:83); and
(3) a V_{L} CDR3 having an amino acid sequence of QQYYX₁YRT (SEQ ID NO:84) wherein X1 is S or I.

78. The antibody of claim 77, wherein the antibody comprises:
(a) a heavy chain variable (V_{H}) region comprising:
(1) a V_{H} CDR1 having an amino acid sequence of SEQ ID NO:111 or SEQ ID NO:112;
(2) a V_{H} CDR2 having an amino acid sequence selected from the group consisting of SEQ ID NO:113, SEQ ID NO:114, SEQ ID NO:115, SEQ ID NO:116, and SEQ ID NO:117; and
(3) a V_{H} CDR3 having an amino acid sequence selected from the group consisting of SEQ ID NO:118, SEQ ID NO:119, SEQ ID NO:120, and SEQ ID NO:121;
and
(b) a light chain variable (V_{L}) region comprising:
(1) a V_{L} CDR1 having an amino acid sequence selected from the group consisting of SEQ ID NO:94, SEQ ID NO:122, and SEQ ID NO:123;
(2) a V_{L} CDR2 having an amino acid sequence of WAS (SEQ ID NO:83); and
(3) a V_{L} CDR3 having an amino acid sequence of SEQ ID NO:124 or SEQ ID NO:125.

79. The antibody of claim 69, wherein the antibody comprises:
(a) a heavy chain variable (V_{H}) region comprising:
(1) a V_{H} CDR1 having an amino acid sequence of GFSLTDYX₁ (SEQ ID NO:85) wherein X₁ is a naturally occurring amino acid;
(2) a V_{H} CDR2 having an amino acid sequence of IWGGGX₁T (SEQ ID NO:86) wherein X₁ is a naturally occurring amino acid; and
(3) a V_{H} CDR3 having an amino acid sequence of AKHEEVSRFAX₁ (SEQ ID NO:87) wherein X₁ is a naturally occurring amino acid; and
(b) a light chain variable (V_{L}) region comprising:
(1) a V_{L} CDR1 having an amino acid sequence of QSLVYSNGNSY (SEQ ID NO:88);
(2) a V_{L} CDR2 having an amino acid sequence of KVS (SEQ ID NO:71); and
(3) a V_{L} CDR3 having an amino acid sequence of SQSTHIPLT (SEQ ID NO:90).

80. The antibody of claim 79, wherein the antibody comprises:
(a) a heavy chain variable (V_{H}) region comprising:
(1) a V_{H} CDR1 having an amino acid sequence of GFSLTDYX₁ (SEQ ID NO:85) wherein X1 is A or G;
(2) a V_{H} CDR2 having an amino acid sequence of IWGGGX₁T (SEQ ID NO:86) wherein X1 is R or G; and
(3) a V_{H} CDR3 having an amino acid sequence of AKHEEVSRFAX₁ (SEQ ID NO:87) wherein X1 is Y or H;
and
(b) a light chain variable (V_{L}) region comprising:
(1) a V_{L} CDR1 having an amino acid sequence of QSLVYSNGNSY (SEQ ID NO:88);
(2) a V_{L} CDR2 having an amino acid sequence of KVS (SEQ ID NO:71); and
(3) a V_{L} CDR3 having an amino acid sequence of SQSTHIPLT (SEQ ID NO:90).

81. The antibody of claim 80, wherein the antibody comprises:
(a) a heavy chain variable (V_{H}) region comprising:
(1) a V_{H} CDR1 having an amino acid sequence of SEQ ID NO:126 or SEQ ID NO:127;
(2) a V_{H} CDR2 having an amino acid sequence of SEQ ID NO:128 or SEQ ID NO:129; and
(3) a V_{H} CDR3 having an amino acid sequence of SEQ ID NO:130 or SEQ ID NO:77;
and
(b) a light chain variable (V_{L}) region comprising:
(1) a V_{L} CDR1 having an amino acid sequence of QSLVYSNGNSY (SEQ ID NO:88);
(2) a V_{L} CDR2 having an amino acid sequence of KVS (SEQ ID NO:71); and
(3) a V_{L} CDR3 having an amino acid sequence of SQSTHIPLT (SEQ ID NO:90).

82. The antibody of claim 69, wherein the antibody comprises:
(a) a heavy chain variable (V_{H}) region comprising:
(1) a V_{H} CDR1 having an amino acid sequence of GFSFNTHA (SEQ ID NO:91);
(2) a V_{H} CDR2 having an amino acid sequence of IRSKSNNYAR (SEQ ID NO:92); and
(3) a V_{H} CDR3 having an amino acid sequence of VKQGDGGFAY (SEQ ID NO:93);
and
(b) a light chain variable (V_{L}) region comprising:
(1) a V_{L} CDR1 having an amino acid sequence of QSLLYSSNQKNY (SEQ ID NO:94);
(2) a V_{L} CDR2 having an amino acid sequence of WAS (SEQ ID NO:83); and
(3) a V_{L} CDR3 having an amino acid sequence of QQYYSYPPT (SEQ ID NO:96).

83. An isolated antibody or functional fragment thereof that binds to C16orf54, wherein the antibody comprises:
(a) a heavy chain variable (V_{H}) region comprising a V_{H} CDR1, a V_{H} CDR2, and a V_{H} CDR3 amino acid sequence depicted in Tables 6-29;
and/or
(b) a light chain variable (V_{L}) region comprising a V_{L} CDR1, a V_{L} CDR2, and a V_{L} CDR3 amino acid sequence depicted in Tables 6, 10, 12-22, 24, 25 and 29.

84. The antibody of claim 83, wherein the antibody comprises a heavy chain variable (V_{H}) region comprising a V_{H} CDR1, a V_{H} CDR2, and a V_{H} CDR3 amino acid sequence depicted in Tables 6-29.

85. The antibody of claim 83, wherein the antibody comprises a light chain variable (V_{L}) region comprising a V_{L} CDR1, a V_{L} CDR2, and a V_{L} CDR3 amino acid sequence depicted in Tables 6, 10, 12-22, 24, 25 and 29.

86. The antibody of claim 83, wherein the antibody comprises:
(a) a heavy chain variable (V_{H}) region comprising:
(1) a V_{H} CDR1 having an amino acid sequence of selected from the group consisting of SEQ ID NO:76, 147, 161, 166, and 172;
(2) a V_{H} CDR2 having an amino acid sequence of selected from the group consisting of SEQ ID NO:97, 148, 162, 167, and 173; and
(3) a V_{H} CDR3 having an amino acid sequence of selected from the group consisting of SEQ ID NO:103, 149, 163, and 168;
and
(b) a light chain variable (V_{L}) region comprising:
(1) a V_{L} CDR1 having an amino acid sequence of selected from the group consisting of SEQ ID NO:106, 150, 164, and 169;
(2) a V_{L} CDR2 having an amino acid sequence of selected from the group consisting of SEQ ID NO:71, 160, and 170; and
(3) a V_{L} CDR3 having an amino acid sequence of selected from the group consisting of SEQ ID NO:109, 165, and 171.

87. The antibody of claim 86, wherein the antibody comprises:
(a) a heavy chain variable (V_{H}) region comprising:
(1) a V_{H} CDR1 having the amino acid sequence of SEQ ID NO:76;
(2) a V_{H} CDR2 having the amino acid sequence of SEQ ID NO:97; and
(3) a V_{H} CDR3 having the amino acid sequence of SEQ ID NO:103; and
(b) a light chain variable (V_{L}) region comprising:
(1) a V_{L} CDR1 having the amino acid sequence of SEQ ID NO:106;
(2) a V_{L} CDR2 having the amino acid sequence of SEQ ID NO:71; and
(3) a V_{L} CDR3 having the amino acid sequence of SEQ ID NO:109.

88. The antibody of claim 86, wherein the antibody comprises:
(a) a heavy chain variable (V_{H}) region comprising:
(1) a V_{H} CDR1 having the amino acid sequence of SEQ ID NO:147;
(2) a V_{H} CDR2 having the amino acid sequence of SEQ ID NO:148;
(3) a V_{H} CDR3 having the amino acid sequence of SEQ ID NO:149; and
(b) a light chain variable (V_{L}) region comprising:
(1) a V_{L} CDR1 having the amino acid sequence of SEQ ID NO:150;
(2) a V_{L} CDR2 having the amino acid sequence of SEQ ID NO:160; and
(3) a V_{L} CDR3 having the amino acid sequence of SEQ ID NO:109.

89. The antibody of claim 86, wherein the antibody comprises:
(a) a heavy chain variable (V_{H}) region comprising:
(1) a V_{H} CDR1 having the amino acid sequence of SEQ ID NO:161;
(2) a V_{H} CDR2 having the amino acid sequence of SEQ ID NO:162; and
(3) a V_{H} CDR3 having the amino acid sequence of SEQ ID NO:163; and
(b) a light chain variable (V_{L}) region comprising:
(1) a V_{L} CDR1 having the amino acid sequence of SEQ ID NO:164;
(2) a V_{L} CDR2 having the amino acid sequence of SEQ ID NO:71; and
(3) a V_{L} CDR3 having the amino acid sequence of SEQ ID NO:165.

90. The antibody of claim 86, wherein the antibody comprises:
(a) a heavy chain variable (V_{H}) region comprising:
(1) a V_{H} CDR1 having the amino acid sequence of SEQ ID NO:166;
(2) a V_{H} CDR2 having the amino acid sequence of SEQ ID NO:167; and
(3) a V_{H} CDR3 having the amino acid sequence of SEQ ID NO:168; and
(b) a light chain variable (V_{L}) region comprising:
(1) a V_{L} CDR1 having the amino acid sequence of SEQ ID NO:169;
(2) a V_{L} CDR2 having the amino acid sequence of SEQ ID NO:170; and
(3) a V_{L} CDR3 having the amino acid sequence of SEQ ID NO:171.

91. The antibody of claim 86, wherein the antibody comprises:
(a) a heavy chain variable (V_{H}) region comprising:
(1) a V_{H} CDR1 having the amino acid sequence of SEQ ID NO:172;
(2) a V_{H} CDR2 having the amino acid sequence of SEQ ID NO:173; and
(3) a V_{H} CDR3 having the amino acid sequence of SEQ ID NO:149; and
(b) a light chain variable (V_{L}) region comprising:
(1) a V_{L} CDR1 having the amino acid sequence of SEQ ID NO:150;
(2) a V_{L} CDR2 having the amino acid sequence of SEQ ID NO:160; and
(3) a V_{L} CDR3 having the amino acid sequence of SEQ ID NO:109.

92. The antibody of claim 83, wherein the antibody comprises:
(a) a heavy chain variable (V_{H}) region comprising:
(1) a V_{H} CDR1 having an amino acid sequence of selected from the group consisting of SEQ ID NO:76, 147, 161, 166, and 172;
(2) a V_{H} CDR2 having an amino acid sequence of selected from the group consisting of SEQ ID NO:97, 148, 162, 167, and 173; and
(3) a V_{H} CDR3 having an amino acid sequence of selected from the group consisting of SEQ ID NO:103, 149, 163, and 168.

93. The antibody of claim 92, wherein the antibody comprises:
(a) a heavy chain variable (V_{H}) region comprising:
(1) a V_{H} CDR1 having the amino acid sequence of SEQ ID NO:76;
(2) a V_{H} CDR2 having the amino acid sequence of SEQ ID NO:97; and
(3) a V_{H} CDR3 having the amino acid sequence of SEQ ID NO:103.

94. The antibody of claim 92, wherein the antibody comprises:
(a) a heavy chain variable (V_{H}) region comprising:
(1) a V_{H} CDR1 having the amino acid sequence of SEQ ID NO:147;
(2) a V_{H} CDR2 having the amino acid sequence of SEQ ID NO:148;
(3) a V_{H} CDR3 having the amino acid sequence of SEQ ID NO:149.

95. The antibody of claim 92, wherein the antibody comprises:
(a) a heavy chain variable (V_{H}) region comprising:
(1) a V_{H} CDR1 having the amino acid sequence of SEQ ID NO:161;
(2) a V_{H} CDR2 having the amino acid sequence of SEQ ID NO:162; and
(3) a V_{H} CDR3 having the amino acid sequence of SEQ ID NO:163.

96. The antibody of claim 92, wherein the antibody comprises:
(a) a heavy chain variable (V_{H}) region comprising:
(1) a V_{H} CDR1 having the amino acid sequence of SEQ ID NO:166;
(2) a V_{H} CDR2 having the amino acid sequence of SEQ ID NO:167; and
(3) a V_{H} CDR3 having the amino acid sequence of SEQ ID NO:168.

97. The antibody of claim 92, wherein the antibody comprises:
(a) a heavy chain variable (V_{H}) region comprising:
(1) a V_{H} CDR1 having the amino acid sequence of SEQ ID NO:172;
(2) a V_{H} CDR2 having the amino acid sequence of SEQ ID NO:173; and
(3) a V_{H} CDR3 having the amino acid sequence of SEQ ID NO:149.

98. The antibody of claim 83, wherein the antibody comprises:
(a) a heavy chain variable (V_{H}) region comprising:
(1) a V_{H} CDR1 having an amino acid sequence of selected from the group consisting of SEQ ID NO:76, 147, 161, 166, and 172;
(2) a V_{H} CDR2 having an amino acid sequence of selected from the group consisting of SEQ ID NO:97, 148, 162, 167, and 173; and
(3) a V_{H} CDR3 having an amino acid sequence of selected from the group consisting of SEQ ID NO:103, 149, 163, and 168.

99. The antibody of claim 98, wherein the antibody comprises:
(a) a heavy chain variable (V_{H}) region comprising:
(1) a V_{H} CDR1 having the amino acid sequence of SEQ ID NO:76;
(2) a V_{H} CDR2 having the amino acid sequence of SEQ ID NO:97; and
(3) a V_{H} CDR3 having the amino acid sequence of SEQ ID NO:103.

100. The antibody of claim 98, wherein the antibody comprises:
(a) a heavy chain variable (V_{H}) region comprising:
(1) a V_{H} CDR1 having the amino acid sequence of SEQ ID NO:147;
(2) a V_{H} CDR2 having the amino acid sequence of SEQ ID NO:148; and
(3) a V_{H} CDR3 having the amino acid sequence of SEQ ID NO:149.

101. The antibody of claim 98, wherein the antibody comprises:
(a) a heavy chain variable (V_{H}) region comprising:
(1) a V_{H} CDR1 having the amino acid sequence of SEQ ID NO:161;
(2) a V_{H} CDR2 having the amino acid sequence of SEQ ID NO:162; and
(3) a V_{H} CDR3 having the amino acid sequence of SEQ ID NO:163.

102. The antibody of claim 98, wherein the antibody comprises:
(a) a heavy chain variable (V_{H}) region comprising:
(1) a V_{H} CDR1 having the amino acid sequence of SEQ ID NO:166;
(2) a V_{H} CDR2 having the amino acid sequence of SEQ ID NO:167; and
(3) a V_{H} CDR3 having the amino acid sequence of SEQ ID NO:168.

103. The antibody of claim 98, wherein the antibody comprises:
(a) a heavy chain variable (V_{H}) region comprising:
(1) a V_{H} CDR1 having the amino acid sequence of SEQ ID NO:172;
(2) a V_{H} CDR2 having the amino acid sequence of SEQ ID NO:173; and
(3) a V_{H} CDR3 having the amino acid sequence of SEQ ID NO:149.

104. The antibody of claim 83, wherein the antibody comprises:
(a) a heavy chain variable (V_{H}) region comprising:
(1) a V_{H} CDR1 having an amino acid sequence of selected from the group consisting of SEQ ID NO:89, 174, 176, 177, and 179;
(2) a V_{H} CDR2 having an amino acid sequence of selected from the group consisting of SEQ ID NO:98, 175, 162, 178, and 180; and
(3) a V_{H} CDR3 having an amino acid sequence of selected from the group consisting of SEQ ID NO:103, 149, 163, and 168.

105. The antibody of claim 104, wherein the antibody comprises:
(a) a heavy chain variable (V_{H}) region comprising:
(1) a V_{H} CDR1 having the amino acid sequence of SEQ ID NO:89;
(2) a V_{H} CDR2 having the amino acid sequence of SEQ ID NO:98; and
(3) a V_{H} CDR3 having the amino acid sequence of SEQ ID NO:103.

106. The antibody of claim 104, wherein the antibody comprises:
(a) a heavy chain variable (V_{H}) region comprising:
(1) a V_{H} CDR1 having the amino acid sequence of SEQ ID NO:174;
(2) a V_{H} CDR2 having the amino acid sequence of SEQ ID NO:175; and
(3) a V_{H} CDR3 having the amino acid sequence of SEQ ID NO:149.

107. The antibody of claim 104, wherein the antibody comprises:
(a) a heavy chain variable (V_{H}) region comprising:
(1) a V_{H} CDR1 having the amino acid sequence of SEQ ID NO:179;
(2) a V_{H} CDR2 having the amino acid sequence of SEQ ID NO:162; and
(3) a V_{H} CDR3 having the amino acid sequence of SEQ ID NO:163.

108. The antibody of claim 104, wherein the antibody comprises:
(a) a heavy chain variable (V_{H}) region comprising:
(1) a V_{H} CDR1 having the amino acid sequence of SEQ ID NO:177;
(2) a V_{H} CDR2 having the amino acid sequence of SEQ ID NO:178;
(3) a V_{H} CDR3 having the amino acid sequence of SEQ ID NO:168.

109. The antibody of claim 104, wherein the antibody comprises:
(a) a heavy chain variable (V_{H}) region comprising:
(1) a V_{H} CDR1 having the amino acid sequence of SEQ ID NO:179;
(2) a V_{H} CDR2 having the amino acid sequence of SEQ ID NO:180; and
(3) a V_{H} CDR3 having the amino acid sequence of SEQ ID NO:149.

110. The antibody of claim 83, wherein the antibody comprises:
(a) a heavy chain variable (V_{H}) region comprising:
(1) a V_{H} CDR1 having an amino acid sequence of selected from the group consisting of SEQ ID NO:95, 181, 184, 186, and 189;
(2) a V_{H} CDR2 having an amino acid sequence of selected from the group consisting of SEQ ID NO:99, 182, 162, 187, and 190; and
(3) a V_{H} CDR3 having an amino acid sequence of selected from the group consisting of SEQ ID NO:103, 149, 163, and 168; and
(b) a light chain variable (V_{L}) region comprising:
(1) a V_{L} CDR1 having an amino acid sequence of selected from the group consisting of SEQ ID NO:107, 183, 185, and 188;
(2) a V_{L} CDR2 having an amino acid sequence of selected from the group consisting of SEQ ID NO:71, 160, and 170; and
(3) a V_{L} CDR3 having an amino acid sequence of selected from the group consisting of SEQ ID NO:109, 165 and 171.

111. The antibody of claim 110, wherein the antibody comprises:
(a) a heavy chain variable (V_{H}) region comprising:
(1) a V_{H} CDR1 having the amino acid sequence of SEQ ID NO:95;
(2) a V_{H} CDR2 having the amino acid sequence of SEQ ID NO:99; and
(3) a V_{H} CDR3 having the amino acid sequence of SEQ ID NO:103; and
(b) a light chain variable (V_{L}) region comprising:
(1) a V_{L} CDR1 having the amino acid sequence of SEQ ID NO:107;
(2) a V_{L} CDR2 having the amino acid sequence of SEQ ID NO:71; and
(3) a V_{L} CDR3 having the amino acid sequence of SEQ ID NO:109.

112. The antibody of claim 110, wherein the antibody comprises:
(a) a heavy chain variable (V_{H}) region comprising:
(1) a V_{H} CDR1 having the amino acid sequence of SEQ ID NO:181;
(2) a V_{H} CDR2 having the amino acid sequence of SEQ ID NO:182; and
(3) a V_{H} CDR3 having the amino acid sequence of SEQ ID NO:149; and
(b) a light chain variable (V_{L}) region comprising:
(1) a V_{L} CDR1 having the amino acid sequence of SEQ ID NO:183;
(2) a V_{L} CDR2 having the amino acid sequence of SEQ ID NO:160; and
(3) a V_{L} CDR3 having the amino acid sequence of SEQ ID NO:109.

113. The antibody of claim 110, wherein the antibody comprises:
(a) a heavy chain variable (V_{H}) region comprising:
(1) a V_{H} CDR1 having the amino acid sequence of SEQ ID NO:184;
(2) a V_{H} CDR2 having the amino acid sequence of SEQ ID NO:162; and
(3) a V_{H} CDR3 having the amino acid sequence of SEQ ID NO:163; and
(b) a light chain variable (V_{L}) region comprising:
(1) a V_{L} CDR1 having the amino acid sequence of SEQ ID NO:185;
(2) a V_{L} CDR2 having the amino acid sequence of SEQ ID NO:71; and
(3) a V_{L} CDR3 having the amino acid sequence of SEQ ID NO:165.

114. The antibody of claim 110, wherein the antibody comprises:
(a) a heavy chain variable (V_{H}) region comprising:
(1) a V_{H} CDR1 having the amino acid sequence of SEQ ID NO:186;
(2) a V_{H} CDR2 having the amino acid sequence of SEQ ID NO:187; and
(3) a V_{H} CDR3 having the amino acid sequence of SEQ ID NO:168; and
(b) a light chain variable (V_{L}) region comprising:
(1) a V_{L} CDR1 having the amino acid sequence of SEQ ID NO:188;
(2) a V_{L} CDR2 having the amino acid sequence of SEQ ID NO:170; and
(3) a V_{L} CDR3 having the amino acid sequence of SEQ ID NO:171.

115. The antibody of claim 110, wherein the antibody comprises:
(a) a heavy chain variable (V_{H}) region comprising:
(1) a V_{H} CDR1 having the amino acid sequence of SEQ ID NO:189;
(2) a V_{H} CDR2 having the amino acid sequence of SEQ ID NO:190; and
(3) a V_{H} CDR3 having the amino acid sequence of SEQ ID NO:149; and
(b) a light chain variable (V_{L}) region comprising:
(1) a V_{L} CDR1 having the amino acid sequence of SEQ ID NO:183;
(2) a V_{L} CDR2 having the amino acid sequence of SEQ ID NO:160; and
(3) a V_{L} CDR3 having the amino acid sequence of SEQ ID NO:109.

116. The antibody of claim 83, wherein the antibody comprises:
(a) a heavy chain variable (V_{H}) region comprising:
(1) a V_{H} CDR1 having an amino acid sequence of selected from the group consisting of SEQ ID NO:96, 191, 193, 195, and 197;
(2) a V_{H} CDR2 having an amino acid sequence of selected from the group consisting of SEQ ID NO:100, 192, 194, 196, and 198; and
(3) a V_{H} CDR3 having an amino acid sequence of selected from the group consisting of SEQ ID NO:103, 149, 163, and 168.

117. The antibody of claim 116, wherein the antibody comprises:
(a) a heavy chain variable (V_{H}) region comprising:
(1) a V_{H} CDR1 having the amino acid sequence of SEQ ID NO:96;
(2) a V_{H} CDR2 having the amino acid sequence of SEQ ID NO:100; and
(3) a V_{H} CDR3 having the amino acid sequence of SEQ ID NO:103.

118. The antibody of claim 116, wherein the antibody comprises:
(a) a heavy chain variable (V_{H}) region comprising:
(1) a V_{H} CDR1 having the amino acid sequence of SEQ ID NO:191;
(2) a V_{H} CDR2 having the amino acid sequence of SEQ ID NO:192; and
(3) a V_{H} CDR3 having the amino acid sequence of SEQ ID NO:149.

119. The antibody of claim 116, wherein the antibody comprises:
(a) a heavy chain variable (V_{H}) region comprising:
(1) a V_{H} CDR1 having the amino acid sequence of SEQ ID NO:193;
(2) a V_{H} CDR2 having the amino acid sequence of SEQ ID NO:194; and
(3) a V_{H} CDR3 having the amino acid sequence of SEQ ID NO:163.

120. The antibody of claim 116, wherein the antibody comprises:
(a) a heavy chain variable (V_{H}) region comprising:
(1) a V_{H} CDR1 having the amino acid sequence of SEQ ID NO:195;
(2) a V_{H} CDR2 having the amino acid sequence of SEQ ID NO:196; and
(3) a V_{H} CDR3 having the amino acid sequence of SEQ ID NO:168.

121. The antibody of claim 116, wherein the antibody comprises:
(a) a heavy chain variable (V_{H}) region comprising:
(1) a V_{H} CDR1 having the amino acid sequence of SEQ ID NO:197;
(2) a V_{H} CDR2 having the amino acid sequence of SEQ ID NO:198; and
(3) a V_{H} CDR3 having the amino acid sequence of SEQ ID NO:149.

122. The antibody of claim 83, wherein the antibody comprises:
(a) a heavy chain variable (V_{H}) region comprising:
(1) a V_{H} CDR1 having an amino acid sequence of selected from the group consisting of SEQ ID NO:89, 199, 176, 202, and 206;
(2) a V_{H} CDR2 having an amino acid sequence of selected from the group consisting of SEQ ID NO:101, 200, 194, 203, and 207; and
(3) a V_{H} CDR3 having an amino acid sequence of selected from the group consisting of SEQ ID NO:104, 149, 163, and 204;
and
(b) a light chain variable (V_{L}) region comprising:
(1) a V_{L} CDR1 having an amino acid sequence of selected from the group consisting of SEQ ID NO:107, 183, 185, and 188;
(2) a V_{L} CDR2 having an amino acid sequence of selected from the group consisting of SEQ ID NO:71, 160 and 170; and
(3) a V_{L} CDR3 having an amino acid sequence of selected from the group consisting of SEQ ID NO:110, 201, and 205.

123. The antibody of claim 122, wherein the antibody comprises:
(a) a heavy chain variable (V_{H}) region comprising:
(1) a V_{H} CDR1 having the amino acid sequence of SEQ ID NO:89;
(2) a V_{H} CDR2 having the amino acid sequence of SEQ ID NO:101; and
(3) a V_{H} CDR3 having the amino acid sequence of SEQ ID NO:104; and
(b) a light chain variable (V_{L}) region comprising:
(1) a V_{L} CDR1 having the amino acid sequence of SEQ ID NO:107;
(2) a V_{L} CDR2 having the amino acid sequence of SEQ ID NO:71; and
(3) a V_{L} CDR3 having the amino acid sequence of SEQ ID NO:110.

124. The antibody of claim 122, wherein the antibody comprises:
(a) a heavy chain variable (V_{H}) region comprising:
(1) a V_{H} CDR1 having the amino acid sequence of SEQ ID NO:199;
(2) a V_{H} CDR2 having the amino acid sequence of SEQ ID NO:200; and
(3) a V_{H} CDR3 having the amino acid sequence of SEQ ID NO:149; and
(b) a light chain variable (V_{L}) region comprising:
(1) a V_{L} CDR1 having the amino acid sequence of SEQ ID NO:183;
(2) a V_{L} CDR2 having the amino acid sequence of SEQ ID NO:160; and
(3) a V_{L} CDR3 having the amino acid sequence of SEQ ID NO:110.

125. The antibody of claim 122, wherein the antibody comprises:
(a) a heavy chain variable (V_{H}) region comprising:
(1) a V_{H} CDR1 having the amino acid sequence of SEQ ID NO:176;
(2) a V_{H} CDR2 having the amino acid sequence of SEQ ID NO:194; and
(3) a V_{H} CDR3 having the amino acid sequence of SEQ ID NO:163; and
(b) a light chain variable (V_{L}) region comprising:
(1) a V_{L} CDR1 having the amino acid sequence of SEQ ID NO:185;
(2) a V_{L} CDR2 having the amino acid sequence of SEQ ID NO:71; and
(3) a V_{L} CDR3 having the amino acid sequence of SEQ ID NO:201.

126. The antibody of claim 122, wherein the antibody comprises:
(a) a heavy chain variable (V_{H}) region comprising:
(1) a V_{H} CDR1 having the amino acid sequence of SEQ ID NO:202;
(2) a V_{H} CDR2 having the amino acid sequence of SEQ ID NO:203; and
(3) a V_{H} CDR3 having the amino acid sequence of SEQ ID NO:204; and
(b) a light chain variable (V_{L}) region comprising:
(1) a V_{L} CDR1 having the amino acid sequence of SEQ ID NO:188;
(2) a V_{L} CDR2 having the amino acid sequence of SEQ ID NO:170; and
(3) a V_{L} CDR3 having the amino acid sequence of SEQ ID NO:205.

127. The antibody of claim 122, wherein the antibody comprises:
(a) a heavy chain variable (V_{H}) region comprising:
(1) a V_{H} CDR1 having the amino acid sequence of SEQ ID NO:206;
(2) a V_{H} CDR2 having the amino acid sequence of SEQ ID NO:207; and
(3) a V_{H} CDR3 having the amino acid sequence of SEQ ID NO:149; and
(b) a light chain variable (V_{L}) region comprising:
(1) a V_{L} CDR1 having the amino acid sequence of SEQ ID NO:183;
(2) a V_{L} CDR2 having the amino acid sequence of SEQ ID NO:160; and
(3) a V_{L} CDR3 having the amino acid sequence of SEQ ID NO:110.

128. The antibody of claim 83, wherein the antibody comprises:
(a) a heavy chain variable (V_{H}) region comprising:
(1) a V_{H} CDR1 having an amino acid sequence of selected from the group consisting of SEQ ID NO:76, 208, 161, 211, and 213;
(2) a V_{H} CDR2 having an amino acid sequence of selected from the group consisting of SEQ ID NO:101, 209, 194, 203, and 207; and
(3) a V_{H} CDR3 having an amino acid sequence of selected from the group consisting of SEQ ID NO:105, 210, 163, and 212; and
(b) a light chain variable (V_{L}) region comprising:
(1) a V_{L} CDR1 having an amino acid sequence of selected from the group consisting of SEQ ID NO:107, 183, 185, and 188;
(2) a V_{L} CDR2 having an amino acid sequence of selected from the group consisting of SEQ ID NO:71, 160, and 170; and
(3) a V_{L} CDR3 having an amino acid sequence of selected from the group consisting of SEQ ID NO:110, 201, and 205.

129. The antibody of claim 128, wherein the antibody comprises:
(a) a heavy chain variable (V_{H}) region comprising:
(1) a V_{H} CDR1 having the amino acid sequence of SEQ ID NO:76;
(2) a V_{H} CDR2 having the amino acid sequence of SEQ ID NO:101; and
(3) a V_{H} CDR3 having the amino acid sequence of SEQ ID NO:105; and
(b) a light chain variable (V_{L}) region comprising:
(1) a V_{L} CDR1 having the amino acid sequence of SEQ ID NO:107;
(2) a V_{L} CDR2 having the amino acid sequence of SEQ ID NO:71; and
(3) a V_{L} CDR3 having the amino acid sequence of SEQ ID NO:110.

130. The antibody of claim 128, wherein the antibody comprises:
(a) a heavy chain variable (V_{H}) region comprising:
(1) a V_{H} CDR1 having the amino acid sequence of SEQ ID NO:208;
(2) a V_{H} CDR2 having the amino acid sequence of SEQ ID NO:209; and
(3) a V_{H} CDR3 having the amino acid sequence of SEQ ID NO:210; and
(b) a light chain variable (V_{L}) region comprising:
(1) a V_{L} CDR1 having the amino acid sequence of SEQ ID NO:183;
(2) a V_{L} CDR2 having the amino acid sequence of SEQ ID NO:160; and
(3) a V_{L} CDR3 having the amino acid sequence of SEQ ID NO:110.

131. The antibody of claim 128, wherein the antibody comprises:
(a) a heavy chain variable (V_{H}) region comprising:
(1) a V_{H} CDR1 having the amino acid sequence of SEQ ID NO:161;
(2) a V_{H} CDR2 having the amino acid sequence of SEQ ID NO:194; and
(3) a V_{H} CDR3 having the amino acid sequence of SEQ ID NO:163; and
(b) a light chain variable (V_{L}) region comprising:
(1) a V_{L} CDR1 having the amino acid sequence of SEQ ID NO:185;
(2) a V_{L} CDR2 having the amino acid sequence of SEQ ID NO:71; and
(3) a V_{L} CDR3 having the amino acid sequence of SEQ ID NO:201.

132. The antibody of claim 128, wherein the antibody comprises:
(a) a heavy chain variable (V_{H}) region comprising:
(1) a V_{H} CDR1 having the amino acid sequence of SEQ ID NO:211;
(2) a V_{H} CDR2 having the amino acid sequence of SEQ ID NO:203; and
(3) a V_{H} CDR3 having the amino acid sequence of SEQ ID NO:212; and
(b) a light chain variable (V_{L}) region comprising:
(1) a V_{L} CDR1 having the amino acid sequence of SEQ ID NO:188;
(2) a V_{L} CDR2 having the amino acid sequence of SEQ ID NO:170; and
(3) a V_{L} CDR3 having the amino acid sequence of SEQ ID NO:205.

133. The antibody of claim 128, wherein the antibody comprises:
(a) a heavy chain variable (V_{H}) region comprising:
(1) a V_{H} CDR1 having the amino acid sequence of SEQ ID NO:213;
(2) a V_{H} CDR2 having the amino acid sequence of SEQ ID NO:207; and
(3) a V_{H} CDR3 having the amino acid sequence of SEQ ID NO:210; and
(b) a light chain variable (V_{L}) region comprising:
(1) a V_{L} CDR1 having the amino acid sequence of SEQ ID NO:183;
(2) a V_{L} CDR2 having the amino acid sequence of SEQ ID NO:160; and
(3) a V_{L} CDR3 having the amino acid sequence of SEQ ID NO:110.

134. The antibody of claim 83, wherein the antibody comprises:
(a) a heavy chain variable (V_{H}) region comprising:
(1) a V_{H} CDR1 having an amino acid sequence of selected from the group consisting of SEQ ID NO:76, 208, 161, 211, and 213;
(2) a V_{H} CDR2 having an amino acid sequence of selected from the group consisting of SEQ ID NO:101, 209, 194, 203, and 207; and
(3) a V_{H} CDR3 having an amino acid sequence of selected from the group consisting of SEQ ID NO:105, 210, 163, and 212;
and
(b) a light chain variable (V_{L}) region comprising:
(1) a V_{L} CDR1 having an amino acid sequence of selected from the group consisting of SEQ ID NO:107, 183, 185, and 188;
(2) a V_{L} CDR2 having an amino acid sequence of selected from the group consisting of SEQ ID NO:71, 160, and 170; and
(3) a V_{L} CDR3 having an amino acid sequence of selected from the group consisting of SEQ ID NO:110, 201, and 205.

135. The antibody of claim 134, wherein the antibody comprises:
(a) a heavy chain variable (V_{H}) region comprising:
(1) a V_{H} CDR1 having the amino acid sequence of SEQ ID NO:76;
(2) a V_{H} CDR2 having the amino acid sequence of SEQ ID NO:101; and
(3) a V_{H} CDR3 having the amino acid sequence of SEQ ID NO:105; and
(b) a light chain variable (V_{L}) region comprising:
(1) a V_{L} CDR1 having the amino acid sequence of SEQ ID NO:107;
(2) a V_{L} CDR2 having the amino acid sequence of SEQ ID NO:71; and
(3) a V_{L} CDR3 having the amino acid sequence of SEQ ID NO:110.

136. The antibody of claim 134, wherein the antibody comprises:
(a) a heavy chain variable (V_{H}) region comprising:
(1) a V_{H} CDR1 having the amino acid sequence of SEQ ID NO:208;
(2) a V_{H} CDR2 having the amino acid sequence of SEQ ID NO:209; and
(3) a V_{H} CDR3 having the amino acid sequence of SEQ ID NO:210; and
(b) a light chain variable (V_{L}) region comprising:
(1) a V_{L} CDR1 having the amino acid sequence of SEQ ID NO:183;
(2) a V_{L} CDR2 having the amino acid sequence of SEQ ID NO:160; and
(3) a V_{L} CDR3 having the amino acid sequence of SEQ ID NO:110.

137. The antibody of claim 134, wherein the antibody comprises:
(a) a heavy chain variable (V_{H}) region comprising:
(1) a V_{H} CDR1 having the amino acid sequence of SEQ ID NO:161;
(2) a V_{H} CDR2 having the amino acid sequence of SEQ ID NO:194; and
(3) a V_{H} CDR3 having the amino acid sequence of SEQ ID NO:163; and
(b) a light chain variable (V_{L}) region comprising:
(1) a V_{L} CDR1 having the amino acid sequence of SEQ ID NO:185;
(2) a V_{L} CDR2 having the amino acid sequence of SEQ ID NO:71; and
(3) a V_{L} CDR3 having the amino acid sequence of SEQ ID NO:201.

138. The antibody of claim 134, wherein the antibody comprises:
(a) a heavy chain variable (V_{H}) region comprising:
(1) a V_{H} CDR1 having the amino acid sequence of SEQ ID NO:211;
(2) a V_{H} CDR2 having the amino acid sequence of SEQ ID NO:203; and
(3) a V_{H} CDR3 having the amino acid sequence of SEQ ID NO:212; and
(b) a light chain variable (V_{L}) region comprising:
(1) a V_{L} CDR1 having the amino acid sequence of SEQ ID NO:188;
(2) a V_{L} CDR2 having the amino acid sequence of SEQ ID NO:170; and
(3) a V_{L} CDR3 having the amino acid sequence of SEQ ID NO:205.

139. The antibody of claim 134, wherein the antibody comprises:
(a) a heavy chain variable (V_{H}) region comprising:
(1) a V_{H} CDR1 having the amino acid sequence of SEQ ID NO:213;
(2) a V_{H} CDR2 having the amino acid sequence of SEQ ID NO:207; and
(3) a V_{H} CDR3 having the amino acid sequence of SEQ ID NO:210; and
(b) a light chain variable (V_{L}) region comprising:
(1) a V_{L} CDR1 having the amino acid sequence of SEQ ID NO:183;
(2) a V_{L} CDR2 having the amino acid sequence of SEQ ID NO:160; and
(3) a V_{L} CDR3 having the amino acid sequence of SEQ ID NO:110.

140. The antibody of claim 83, wherein the antibody comprises:
(a) a heavy chain variable (V_{H}) region comprising:
(1) a V_{H} CDR1 having an amino acid sequence of selected from the group consisting of SEQ ID NO:76, 208, 161, 211, and 213;
(2) a V_{H} CDR2 having an amino acid sequence of selected from the group consisting of SEQ ID NO:101, 209, 194, 203, and 207; and
(3) a V_{H} CDR3 having an amino acid sequence of selected from the group consisting of SEQ ID NO:105, 210, 163, and 212;
and
(b) a light chain variable (V_{L}) region comprising:
(1) a V_{L} CDR1 having an amino acid sequence of selected from the group consisting of SEQ ID NO:107, 183, 185, and 188;
(2) a V_{L} CDR2 having an amino acid sequence of selected from the group consisting of SEQ ID NO:71, 160 and 170; and
(3) a V_{L} CDR3 having an amino acid sequence of selected from the group consisting of SEQ ID NO:110, 201 and 205.

141. The antibody of claim 140, wherein the antibody comprises:
(a) a heavy chain variable (V_{H}) region comprising:
(1) a V_{H} CDR1 having the amino acid sequence of SEQ ID NO:76;
(2) a V_{H} CDR2 having the amino acid sequence of SEQ ID NO:101; and
(3) a V_{H} CDR3 having the amino acid sequence of SEQ ID NO:105; and
(b) a light chain variable (V_{L}) region comprising:
(1) a V_{L} CDR1 having the amino acid sequence of SEQ ID NO:107;
(2) a V_{L} CDR2 having the amino acid sequence of SEQ ID NO:71; and
(3) a V_{L} CDR3 having the amino acid sequence of SEQ ID NO:110.

142. The antibody of claim 140, wherein the antibody comprises:
(a) a heavy chain variable (V_{H}) region comprising:
(1) a V_{H} CDR1 having the amino acid sequence of SEQ ID NO:208;
(2) a V_{H} CDR2 having the amino acid sequence of SEQ ID NO:209; and
(3) a V_{H} CDR3 having the amino acid sequence of SEQ ID NO:210; and
(b) a light chain variable (V_{L}) region comprising:
(1) a V_{L} CDR1 having the amino acid sequence of SEQ ID NO:183;
(2) a V_{L} CDR2 having the amino acid sequence of SEQ ID NO:160; and
(3) a V_{L} CDR3 having the amino acid sequence of SEQ ID NO:110.

143. The antibody of claim 140, wherein the antibody comprises:
(a) a heavy chain variable (V_{H}) region comprising:
(1) a V_{H} CDR1 having the amino acid sequence of SEQ ID NO:161;
(2) a V_{H} CDR2 having the amino acid sequence of SEQ ID NO:194; and
(3) a V_{H} CDR3 having the amino acid sequence of SEQ ID NO:163; and
(b) a light chain variable (V_{L}) region comprising:
(1) a V_{L} CDR1 having the amino acid sequence of SEQ ID NO:185;
(2) a V_{L} CDR2 having the amino acid sequence of SEQ ID NO:71; and
(3) a V_{L} CDR3 having the amino acid sequence of SEQ ID NO:201.

144. The antibody of claim 140, wherein the antibody comprises:
(a) a heavy chain variable (V_{H}) region comprising:
(1) a V_{H} CDR1 having the amino acid sequence of SEQ ID NO:211;
(2) a V_{H} CDR2 having the amino acid sequence of SEQ ID NO:203; and
(3) a V_{H} CDR3 having the amino acid sequence of SEQ ID NO:212; and
(b) a light chain variable (V_{L}) region comprising:
(1) a V_{L} CDR1 having the amino acid sequence of SEQ ID NO:188;
(2) a V_{L} CDR2 having the amino acid sequence of SEQ ID NO:170; and
(3) a V_{L} CDR3 having the amino acid sequence of SEQ ID NO:205.

145. The antibody of claim 140, wherein the antibody comprises:
(a) a heavy chain variable (V_{H}) region comprising:
(1) a V_{H} CDR1 having the amino acid sequence of SEQ ID NO:213;
(2) a V_{H} CDR2 having the amino acid sequence of SEQ ID NO:207; and
(3) a V_{H} CDR3 having the amino acid sequence of SEQ ID NO:210; and
(b) a light chain variable (V_{L}) region comprising:
(1) a V_{L} CDR1 having the amino acid sequence of SEQ ID NO:183;
(2) a V_{L} CDR2 having the amino acid sequence of SEQ ID NO:160; and
(3) a V_{L} CDR3 having the amino acid sequence of SEQ ID NO:110.

146. The antibody of claim 83, wherein the antibody comprises:
(a) a heavy chain variable (V_{H}) region comprising:
(1) a V_{H} CDR1 having an amino acid sequence of selected from the group consisting of SEQ ID NO:76, 208, 161, 211, and 213;
(2) a V_{H} CDR2 having an amino acid sequence of selected from the group consisting of SEQ ID NO:102, 214, 194, 164, and 218; and
(3) a V_{H} CDR3 having an amino acid sequence of selected from the group consisting of SEQ ID NO:105, 210, 163, and 212;
and
(b) a light chain variable (V_{L}) region comprising:
(1) a V_{L} CDR1 having an amino acid sequence of selected from the group consisting of SEQ ID NO:108, 215, 216, and 217;
(2) a V_{L} CDR2 having an amino acid sequence of selected from the group consisting of SEQ ID NO:71, 160, and 170; and
(3) a V_{L} CDR3 having an amino acid sequence of selected from the group consisting of SEQ ID NO:110, 201, and 205.

147. The antibody of claim 146, wherein the antibody comprises:
(a) a heavy chain variable (V_{H}) region comprising:
(1) a V_{H} CDR1 having the amino acid sequence of SEQ ID NO:76;
(2) a V_{H} CDR2 having the amino acid sequence of SEQ ID NO:102; and
(3) a V_{H} CDR3 having the amino acid sequence of SEQ ID NO:105; and
(b) a light chain variable (V_{L}) region comprising:
(1) a V_{L} CDR1 having the amino acid sequence of SEQ ID NO:108;
(2) a V_{L} CDR2 having the amino acid sequence of SEQ ID NO:71; and
(3) a V_{L} CDR3 having the amino acid sequence of SEQ ID NO:110.

148. The antibody of claim 146, wherein the antibody comprises:
(a) a heavy chain variable (V_{H}) region comprising:
(1) a V_{H} CDR1 having the amino acid sequence of SEQ ID NO:208;
(2) a V_{H} CDR2 having the amino acid sequence of SEQ ID NO:214; and
(3) a V_{H} CDR3 having the amino acid sequence of SEQ ID NO:210; and
(b) a light chain variable (V_{L}) region comprising:
(1) a V_{L} CDR1 having the amino acid sequence of SEQ ID NO:215;
(2) a V_{L} CDR2 having the amino acid sequence of SEQ ID NO:160; and
(3) a V_{L} CDR3 having the amino acid sequence of SEQ ID NO:110.

149. The antibody of claim 146, wherein the antibody comprises:
(a) a heavy chain variable (V_{H}) region comprising:
(1) a V_{H} CDR1 having the amino acid sequence of SEQ ID NO:161;
(2) a V_{H} CDR2 having the amino acid sequence of SEQ ID NO:194; and
(3) a V_{H} CDR3 having the amino acid sequence of SEQ ID NO:163; and
(b) a light chain variable (V_{L}) region comprising:
(1) a V_{L} CDR1 having the amino acid sequence of SEQ ID NO:216;
(2) a V_{L} CDR2 having the amino acid sequence of SEQ ID NO:71; and
(3) a V_{L} CDR3 having the amino acid sequence of SEQ ID NO:201.

150. The antibody of claim 146, wherein the antibody comprises:
(a) a heavy chain variable (V_{H}) region comprising:
(1) a V_{H} CDR1 having the amino acid sequence of SEQ ID NO:211;
(2) a V_{H} CDR2 having the amino acid sequence of SEQ ID NO:164; and
(3) a V_{H} CDR3 having the amino acid sequence of SEQ ID NO:212; and
(b) a light chain variable (V_{L}) region comprising:
(1) a V_{L} CDR1 having the amino acid sequence of SEQ ID NO:217;
(2) a V_{L} CDR2 having the amino acid sequence of SEQ ID NO:170; and
(3) a V_{L} CDR3 having the amino acid sequence of SEQ ID NO:205.

151. The antibody of claim 146, wherein the antibody comprises:
(a) a heavy chain variable (V_{H}) region comprising:
(1) a V_{H} CDR1 having the amino acid sequence of SEQ ID NO:213;
(2) a V_{H} CDR2 having the amino acid sequence of SEQ ID NO:218; and
(3) a V_{H} CDR3 having the amino acid sequence of SEQ ID NO:210; and
(b) a light chain variable (V_{L}) region comprising:
(1) a V_{L} CDR1 having the amino acid sequence of SEQ ID NO:215;
(2) a V_{L} CDR2 having the amino acid sequence of SEQ ID NO:160; and
(3) a V_{L} CDR3 having the amino acid sequence of SEQ ID NO:110.

152. The antibody of claim 83, wherein the antibody comprises:
(a) a heavy chain variable (V_{H}) region comprising:
(1) a V_{H} CDR1 having an amino acid sequence of selected from the group consisting of SEQ ID NO:76, 208, 161, 211, and 213;
(2) a V_{H} CDR2 having an amino acid sequence of selected from the group consisting of SEQ ID NO:102, 214, 194, 164, and 218; and
(3) a V_{H} CDR3 having an amino acid sequence of selected from the group consisting of SEQ ID NO:105, 210, 163, and 212;
and
(b) a light chain variable (V_{L}) region comprising:
(1) a V_{L} CDR1 having an amino acid sequence of selected from the group consisting of SEQ ID NO:108, 215, 216, and 217;
(2) a V_{L} CDR2 having an amino acid sequence of selected from the group consisting of SEQ ID NO:71, 160, and 170; and
(3) a V_{L} CDR3 having an amino acid sequence of selected from the group consisting of SEQ ID NO:110, 201, and 205.

153. The antibody of claim 152, wherein the antibody comprises:
(a) a heavy chain variable (V_{H}) region comprising:
(1) a V_{H} CDR1 having the amino acid sequence of SEQ ID NO:76;
(2) a V_{H} CDR2 having the amino acid sequence of SEQ ID NO:102; and
(3) a V_{H} CDR3 having the amino acid sequence of SEQ ID NO:105; and
(b) a light chain variable (V_{L}) region comprising:
(1) a V_{L} CDR1 having the amino acid sequence of SEQ ID NO:108;
(2) a V_{L} CDR2 having the amino acid sequence of SEQ ID NO:71; and
(3) a V_{L} CDR3 having the amino acid sequence of SEQ ID NO:110.

154. The antibody of claim 152, wherein the antibody comprises:
(a) a heavy chain variable (V_{H}) region comprising:
(1) a V_{H} CDR1 having the amino acid sequence of SEQ ID NO:208;
(2) a V_{H} CDR2 having the amino acid sequence of SEQ ID NO:214; and
(3) a V_{H} CDR3 having the amino acid sequence of SEQ ID NO:210; and
(b) a light chain variable (V_{L}) region comprising:
(1) a V_{L} CDR1 having the amino acid sequence of SEQ ID NO:215;
(2) a V_{L} CDR2 having the amino acid sequence of SEQ ID NO:160; and
(3) a V_{L} CDR3 having the amino acid sequence of SEQ ID NO:110.

155. The antibody of claim 152, wherein the antibody comprises:
(a) a heavy chain variable (V_{H}) region comprising:
(1) a V_{H} CDR1 having the amino acid sequence of SEQ ID NO:161;
(2) a V_{H} CDR2 having the amino acid sequence of SEQ ID NO:194; and
(3) a V_{H} CDR3 having the amino acid sequence of SEQ ID NO:163; and
(b) a light chain variable (V_{L}) region comprising:
(1) a V_{L} CDR1 having the amino acid sequence of SEQ ID NO:216;
(2) a V_{L} CDR2 having the amino acid sequence of SEQ ID NO:71; and
(3) a V_{L} CDR3 having the amino acid sequence of SEQ ID NO:201.

156. The antibody of claim 152, wherein the antibody comprises:
(a) a heavy chain variable (V_{H}) region comprising:
(1) a V_{H} CDR1 having the amino acid sequence of SEQ ID NO:211;
(2) a V_{H} CDR2 having the amino acid sequence of SEQ ID NO:164; and
(3) a V_{H} CDR3 having the amino acid sequence of SEQ ID NO:212; and
(b) a light chain variable (V_{L}) region comprising:
(1) a V_{L} CDR1 having the amino acid sequence of SEQ ID NO:217;
(2) a V_{L} CDR2 having the amino acid sequence of SEQ ID NO:170; and
(3) a V_{L} CDR3 having the amino acid sequence of SEQ ID NO:205.

157. The antibody of claim 152, wherein the antibody comprises:
(a) a heavy chain variable (V_{H}) region comprising:
(1) a V_{H} CDR1 having the amino acid sequence of SEQ ID NO:213;
(2) a V_{H} CDR2 having the amino acid sequence of SEQ ID NO:218; and
(3) a V_{H} CDR3 having the amino acid sequence of SEQ ID NO:210; and
(b) a light chain variable (V_{L}) region comprising:
(1) a V_{L} CDR1 having the amino acid sequence of SEQ ID NO:215;
(2) a V_{L} CDR2 having the amino acid sequence of SEQ ID NO:160; and
(3) a V_{L} CDR3 having the amino acid sequence of SEQ ID NO:110.

158. The antibody of claim 83, wherein the antibody comprises:
(a) a heavy chain variable (V_{H}) region comprising:
(1) a V_{H} CDR1 having an amino acid sequence of selected from the group consisting of SEQ ID NO:73, 219, 224, 229, and 318;
(2) a V_{H} CDR2 having an amino acid sequence of selected from the group consisting of SEQ ID NO:74, 220, 225, 230, and 319; and
(3) a V_{H} CDR3 having an amino acid sequence of selected from the group consisting of SEQ ID NO: 75, 221, 226, and 231;
and
(b) a light chain variable (V_{L}) region comprising:
(1) a V_{L} CDR1 having an amino acid sequence of selected from the group consisting of SEQ ID NO:76, 222, 227, and 232;
(2) a V_{L} CDR2 having an amino acid sequence of selected from the group consisting of SEQ ID NO:71, 160, and 170; and
(3) a V_{L} CDR3 having an amino acid sequence of selected from the group consisting of SEQ ID NO:78, 228, and 233.

159. The antibody of claim 158, wherein the antibody comprises:
(a) a heavy chain variable (V_{H}) region comprising:
(1) a V_{H} CDR1 having the amino acid sequence of SEQ ID NO:73;
(2) a V_{H} CDR2 having the amino acid sequence of SEQ ID NO:74; and
(3) a V_{H} CDR3 having the amino acid sequence of SEQ ID NO:75; and
(b) a light chain variable (V_{L}) region comprising:
(1) a V_{L} CDR1 having the amino acid sequence of SEQ ID NO:76;
(2) a V_{L} CDR2 having the amino acid sequence of SEQ ID NO:71; and
(3) a V_{L} CDR3 having the amino acid sequence of SEQ ID NO:78.

160. The antibody of claim 158, wherein the antibody comprises:
(a) a heavy chain variable (V_{H}) region comprising:
(1) a V_{H} CDR1 having the amino acid sequence of SEQ ID NO:219;
(2) a V_{H} CDR2 having the amino acid sequence of SEQ ID NO:220; and
(3) a V_{H} CDR3 having the amino acid sequence of SEQ ID NO:221; and
(b) a light chain variable (V_{L}) region comprising:
(1) a V_{L} CDR1 having the amino acid sequence of SEQ ID NO:222;
(2) a V_{L} CDR2 having the amino acid sequence of SEQ ID NO:160; and
(3) a V_{L} CDR3 having the amino acid sequence of SEQ ID NO:78.

161. The antibody of claim 158, wherein the antibody comprises:
(a) a heavy chain variable (V_{H}) region comprising:
(1) a V_{H} CDR1 having the amino acid sequence of SEQ ID NO:224;
(2) a V_{H} CDR2 having the amino acid sequence of SEQ ID NO:225; and
(3) a V_{H} CDR3 having the amino acid sequence of SEQ ID NO:226; and
(b) a light chain variable (V_{L}) region comprising:
(1) a V_{L} CDR1 having the amino acid sequence of SEQ ID NO:227;
(2) a V_{L} CDR2 having the amino acid sequence of SEQ ID NO:71; and
(3) a V_{L} CDR3 having the amino acid sequence of SEQ ID NO:228.

162. The antibody of claim 158, wherein the antibody comprises:
(a) a heavy chain variable (V_{H}) region comprising:
(1) a V_{H} CDR1 having the amino acid sequence of SEQ ID NO:229;
(2) a V_{H} CDR2 having the amino acid sequence of SEQ ID NO:230; and
(3) a V_{H} CDR3 having the amino acid sequence of SEQ ID NO:231; and
(b) a light chain variable (V_{L}) region comprising:
(1) a V_{L} CDR1 having the amino acid sequence of SEQ ID NO:232;
(2) a V_{L} CDR2 having the amino acid sequence of SEQ ID NO:170; and
(3) a V_{L} CDR3 having the amino acid sequence of SEQ ID NO:233.

163. The antibody of claim 158, wherein the antibody comprises:
(a) a heavy chain variable (V_{H}) region comprising:
(1) a V_{H} CDR1 having the amino acid sequence of SEQ ID NO:318;
(2) a V_{H} CDR2 having the amino acid sequence of SEQ ID NO:319; and
(3) a V_{H} CDR3 having the amino acid sequence of SEQ ID NO:221; and
(b) a light chain variable (V_{L}) region comprising:
(1) a V_{L} CDR1 having the amino acid sequence of SEQ ID NO:222;
(2) a V_{L} CDR2 having the amino acid sequence of SEQ ID NO:160; and
(3) a V_{L} CDR3 having the amino acid sequence of SEQ ID NO:78.

164. The antibody of claim 83, wherein the antibody comprises:
(a) a heavy chain variable (V_{H}) region comprising:
(1) a V_{H} CDR1 having an amino acid sequence of selected from the group consisting of SEQ ID NO:111, 234, 240, 244, and 250;
(2) a V_{H} CDR2 having an amino acid sequence of selected from the group consisting of SEQ ID NO:113, 235, 239, 245, and 251; and
(3) a V_{H} CDR3 having an amino acid sequence of selected from the group consisting of SEQ ID NO:118, 236, 241, and 246;
and
(b) a light chain variable (V_{L}) region comprising:
(1) a V_{L} CDR1 having an amino acid sequence of selected from the group consisting of SEQ ID NO:94, 237, 242, and 247;
(2) a V_{L} CDR2 having an amino acid sequence of selected from the group consisting of SEQ ID NO:83, 238, and 248; and
(3) a V_{L} CDR3 having an amino acid sequence of selected from the group consisting of SEQ ID NO:124, 243, and 249.

165. The antibody of claim 164, wherein the antibody comprises:
(a) a heavy chain variable (V_{H}) region comprising:
(1) a V_{H} CDR1 having the amino acid sequence of SEQ ID NO:111;
(2) a V_{H} CDR2 having the amino acid sequence of SEQ ID NO:113; and
(3) a V_{H} CDR3 having the amino acid sequence of SEQ ID NO:118; and
(b) a light chain variable (V_{L}) region comprising:
(1) a V_{L} CDR1 having the amino acid sequence of SEQ ID NO:94;
(2) a V_{L} CDR2 having the amino acid sequence of SEQ ID NO:83; and
(3) a V_{L} CDR3 having the amino acid sequence of SEQ ID NO:124.

166. The antibody of claim 164, wherein the antibody comprises:
(a) a heavy chain variable (V_{H}) region comprising:
(1) a V_{H} CDR1 having the amino acid sequence of SEQ ID NO:234;
(2) a V_{H} CDR2 having the amino acid sequence of SEQ ID NO:235; and
(3) a V_{H} CDR3 having the amino acid sequence of SEQ ID NO:236; and
(b) a light chain variable (V_{L}) region comprising:
(1) a V_{L} CDR1 having the amino acid sequence of SEQ ID NO:237;
(2) a V_{L} CDR2 having the amino acid sequence of SEQ ID NO:238; and
(3) a V_{L} CDR3 having the amino acid sequence of SEQ ID NO:124.

167. The antibody of claim 164, wherein the antibody comprises:
(a) a heavy chain variable (V_{H}) region comprising:
(1) a V_{H} CDR1 having the amino acid sequence of SEQ ID NO:240;
(2) a V_{H} CDR2 having the amino acid sequence of SEQ ID NO:239; and
(3) a V_{H} CDR3 having the amino acid sequence of SEQ ID NO:241; and
(b) a light chain variable (V_{L}) region comprising:
(1) a V_{L} CDR1 having the amino acid sequence of SEQ ID NO:242;
(2) a V_{L} CDR2 having the amino acid sequence of SEQ ID NO:83; and
(3) a V_{L} CDR3 having the amino acid sequence of SEQ ID NO:243.

168. The antibody of claim 164, wherein the antibody comprises:
(a) a heavy chain variable (V_{H}) region comprising:
(1) a V_{H} CDR1 having the amino acid sequence of SEQ ID NO:244;
(2) a V_{H} CDR2 having the amino acid sequence of SEQ ID NO:245; and
(3) a V_{H} CDR3 having the amino acid sequence of SEQ ID NO:246; and
(b) a light chain variable (V_{L}) region comprising:
(1) a V_{L} CDR1 having the amino acid sequence of SEQ ID NO:247;
(2) a V_{L} CDR2 having the amino acid sequence of SEQ ID NO:248; and
(3) a V_{L} CDR3 having the amino acid sequence of SEQ ID NO:249.

169. The antibody of claim 164, wherein the antibody comprises:
(a) a heavy chain variable (V_{H}) region comprising:
(1) a V_{H} CDR1 having the amino acid sequence of SEQ ID NO:250;
(2) a V_{H} CDR2 having the amino acid sequence of SEQ ID NO:251; and
(3) a V_{H} CDR3 having the amino acid sequence of SEQ ID NO:236; and
(b) a light chain variable (V_{L}) region comprising:
(1) a V_{L} CDR1 having the amino acid sequence of SEQ ID NO:237;
(2) a V_{L} CDR2 having the amino acid sequence of SEQ ID NO:238; and
(3) a V_{L} CDR3 having the amino acid sequence of SEQ ID NO:124.

170. The antibody of claim 83, wherein the antibody comprises:
(a) a heavy chain variable (V_{H}) region comprising:
(1) a V_{H} CDR1 having an amino acid sequence of selected from the group consisting of SEQ ID NO:111, 234, 240, 244, and 250;
(2) a V_{H} CDR2 having an amino acid sequence of selected from the group consisting of SEQ ID NO:114, 223, 239, 252, and 253; and
(3) a V_{H} CDR3 having an amino acid sequence of selected from the group consisting of SEQ ID NO:118, 236, 241, and 246;
and
(b) a light chain variable (V_{L}) region comprising:
(1) a V_{L} CDR1 having an amino acid sequence of selected from the group consisting of SEQ ID NO:94, 237, 242, and 247;
(2) a V_{L} CDR2 having an amino acid sequence of selected from the group consisting of SEQ ID NO:83, 238, and 248; and
(3) a V_{L} CDR3 having an amino acid sequence of selected from the group consisting of SEQ ID NO:124, 243 and 249.

171. The antibody of claim 170, wherein the antibody comprises:
(a) a heavy chain variable (V_{H}) region comprising:
(1) a V_{H} CDR1 having the amino acid sequence of SEQ ID NO:111;
(2) a V_{H} CDR2 having the amino acid sequence of SEQ ID NO:114; and
(3) a V_{H} CDR3 having the amino acid sequence of SEQ ID NO:118; and
(b) a light chain variable (V_{L}) region comprising:
(1) a V_{L} CDR1 having the amino acid sequence of SEQ ID NO:94;
(2) a V_{L} CDR2 having the amino acid sequence of SEQ ID NO:83; and
(3) a V_{L} CDR3 having the amino acid sequence of SEQ ID NO:124.

172. The antibody of claim 170, wherein the antibody comprises:
(a) a heavy chain variable (V_{H}) region comprising:
(1) a V_{H} CDR1 having the amino acid sequence of SEQ ID NO:234;
(2) a V_{H} CDR2 having the amino acid sequence of SEQ ID NO:223; and
(3) a V_{H} CDR3 having the amino acid sequence of SEQ ID NO:236; and
(b) a light chain variable (V_{L}) region comprising:
(1) a V_{L} CDR1 having the amino acid sequence of SEQ ID NO:237;
(2) a V_{L} CDR2 having the amino acid sequence of SEQ ID NO:238; and
(3) a V_{L} CDR3 having the amino acid sequence of SEQ ID NO:124.

173. The antibody of claim 170, wherein the antibody comprises:
(a) a heavy chain variable (V_{H}) region comprising:
(1) a V_{H} CDR1 having the amino acid sequence of SEQ ID NO:240;
(2) a V_{H} CDR2 having the amino acid sequence of SEQ ID NO:239; and
(3) a V_{H} CDR3 having the amino acid sequence of SEQ ID NO:241; and
(b) a light chain variable (V_{L}) region comprising:
(1) a V_{L} CDR1 having the amino acid sequence of SEQ ID NO:242;
(2) a V_{L} CDR2 having the amino acid sequence of SEQ ID NO:83; and
(3) a V_{L} CDR3 having the amino acid sequence of SEQ ID NO:243.

174. The antibody of claim 170, wherein the antibody comprises:
(a) a heavy chain variable (V_{H}) region comprising:
(1) a V_{H} CDR1 having the amino acid sequence of SEQ ID NO:244;
(2) a V_{H} CDR2 having the amino acid sequence of SEQ ID NO:252; and
(3) a V_{H} CDR3 having the amino acid sequence of SEQ ID NO:246; and
(b) a light chain variable (V_{L}) region comprising:
(1) a V_{L} CDR1 having the amino acid sequence of SEQ ID NO:247;
(2) a V_{L} CDR2 having the amino acid sequence of SEQ ID NO:248; and
(3) a V_{L} CDR3 having the amino acid sequence of SEQ ID NO:249.

175. The antibody of claim 170, wherein the antibody comprises:
(a) a heavy chain variable (V_{H}) region comprising:
(1) a V_{H} CDR1 having the amino acid sequence of SEQ ID NO:250;
(2) a V_{H} CDR2 having the amino acid sequence of SEQ ID NO:253; and
(3) a V_{H} CDR3 having the amino acid sequence of SEQ ID NO:236; and
(b) a light chain variable (V_{L}) region comprising:
(1) a V_{L} CDR1 having the amino acid sequence of SEQ ID NO:237;
(2) a V_{L} CDR2 having the amino acid sequence of SEQ ID NO:238; and
(3) a V_{L} CDR3 having the amino acid sequence of SEQ ID NO:124.

176. The antibody of claim 83, wherein the antibody comprises:
(a) a heavy chain variable (V_{H}) region comprising:
(1) a V_{H} CDR1 having an amino acid sequence of selected from the group consisting of SEQ ID NO:111, 234, 240, 244, and 250;
(2) a V_{H} CDR2 having an amino acid sequence of selected from the group consisting of SEQ ID NO:115, 254, 239, 259, and 262; and
(3) a V_{H} CDR3 having an amino acid sequence of selected from the group consisting of SEQ ID NO:119, 255, 257, and 260;
and
(b) a light chain variable (V_{L}) region comprising:
(1) a V_{L} CDR1 having an amino acid sequence of selected from the group consisting of SEQ ID NO:122, 256, 258, and 261;
(2) a V_{L} CDR2 having an amino acid sequence of selected from the group consisting of SEQ ID NO:83, 238, and 248; and
(3) a V_{L} CDR3 having an amino acid sequence of selected from the group consisting of SEQ ID NO:124, 243, and 249.

177. The antibody of claim 176, wherein the antibody comprises:
(a) a heavy chain variable (V_{H}) region comprising:
(1) a V_{H} CDR1 having the amino acid sequence of SEQ ID NO:111;
(2) a V_{H} CDR2 having the amino acid sequence of SEQ ID NO:115; and
(3) a V_{H} CDR3 having the amino acid sequence of SEQ ID NO:119; and
(b) a light chain variable (V_{L}) region comprising:
(1) a V_{L} CDR1 having the amino acid sequence of SEQ ID NO:122;
(2) a V_{L} CDR2 having the amino acid sequence of SEQ ID NO:83; and
(3) a V_{L} CDR3 having the amino acid sequence of SEQ ID NO:124.

178. The antibody of claim 176, wherein the antibody comprises:
(a) a heavy chain variable (V_{H}) region comprising:
(1) a V_{H} CDR1 having the amino acid sequence of SEQ ID NO:234;
(2) a V_{H} CDR2 having the amino acid sequence of SEQ ID NO:254; and
(3) a V_{H} CDR3 having the amino acid sequence of SEQ ID NO:255; and
(b) a light chain variable (V_{L}) region comprising:
(1) a V_{L} CDR1 having the amino acid sequence of SEQ ID NO:256;
(2) a V_{L} CDR2 having the amino acid sequence of SEQ ID NO:238; and
(3) a V_{L} CDR3 having the amino acid sequence of SEQ ID NO:124.

179. The antibody of claim 176, wherein the antibody comprises:
(a) a heavy chain variable (V_{H}) region comprising:
(1) a V_{H} CDR1 having the amino acid sequence of SEQ ID NO:240;
(2) a V_{H} CDR2 having the amino acid sequence of SEQ ID NO:239; and
(3) a V_{H} CDR3 having the amino acid sequence of SEQ ID NO:257; and
(b) a light chain variable (V_{L}) region comprising:
(1) a V_{L} CDR1 having the amino acid sequence of SEQ ID NO:258;
(2) a V_{L} CDR2 having the amino acid sequence of SEQ ID NO:83; and
(3) a V_{L} CDR3 having the amino acid sequence of SEQ ID NO:243.

180. The antibody of claim 176, wherein the antibody comprises:
(a) a heavy chain variable (V_{H}) region comprising:
(1) a V_{H} CDR1 having the amino acid sequence of SEQ ID NO:244;
(2) a V_{H} CDR2 having the amino acid sequence of SEQ ID NO:259; and
(3) a V_{H} CDR3 having the amino acid sequence of SEQ ID NO:260; and
(b) a light chain variable (V_{L}) region comprising:
(1) a V_{L} CDR1 having the amino acid sequence of SEQ ID NO:261;
(2) a V_{L} CDR2 having the amino acid sequence of SEQ ID NO:248; and
(3) a V_{L} CDR3 having the amino acid sequence of SEQ ID NO:249.

181. The antibody of claim 176, wherein the antibody comprises:
(a) a heavy chain variable (V_{H}) region comprising:
(1) a V_{H} CDR1 having the amino acid sequence of SEQ ID NO:250;
(2) a V_{H} CDR2 having the amino acid sequence of SEQ ID NO:262; and
(3) a V_{H} CDR3 having the amino acid sequence of SEQ ID NO:255; and
(b) a light chain variable (V_{L}) region comprising:
(1) a V_{L} CDR1 having the amino acid sequence of SEQ ID NO:256;
(2) a V_{L} CDR2 having the amino acid sequence of SEQ ID NO:238; and
(3) a V_{L} CDR3 having the amino acid sequence of SEQ ID NO:124.

182. The antibody of claim 83, wherein the antibody comprises:
(a) a heavy chain variable (V_{H}) region comprising:
(1) a V_{H} CDR1 having an amino acid sequence of selected from the group consisting of SEQ ID NO:111, 234, 240, 244, and 250;
(2) a V_{H} CDR2 having an amino acid sequence of selected from the group consisting of SEQ ID NO:116, 263, 239, 270, and 273; and
(3) a V_{H} CDR3 having an amino acid sequence of selected from the group consisting of SEQ ID NO:120, 264, 267, and 271;
and
(b) a light chain variable (V_{L}) region comprising:
(1) a V_{L} CDR1 having an amino acid sequence of selected from the group consisting of SEQ ID NO:123, 265, 268, and 247;
(2) a V_{L} CDR2 having an amino acid sequence of selected from the group consisting of SEQ ID NO:83, 238, and 248; and
(3) a V_{L} CDR3 having an amino acid sequence of selected from the group consisting of SEQ ID NO:125, 269, and 272.

183. The antibody of claim 182, wherein the antibody comprises:
(a) a heavy chain variable (V_{H}) region comprising:
(1) a V_{H} CDR1 having the amino acid sequence of SEQ ID NO:111;
(2) a V_{H} CDR2 having the amino acid sequence of SEQ ID NO:116; and
(3) a V_{H} CDR3 having the amino acid sequence of SEQ ID NO:120; and
(b) a light chain variable (V_{L}) region comprising:
(1) a V_{L} CDR1 having the amino acid sequence of SEQ ID NO:123;
(2) a V_{L} CDR2 having the amino acid sequence of SEQ ID NO:83; and
(3) a V_{L} CDR3 having the amino acid sequence of SEQ ID NO:125.

184. The antibody of claim 182, wherein the antibody comprises:
(a) a heavy chain variable (V_{H}) region comprising:
(1) a V_{H} CDR1 having the amino acid sequence of SEQ ID NO:234;
(2) a V_{H} CDR2 having the amino acid sequence of SEQ ID NO:263; and
(3) a V_{H} CDR3 having the amino acid sequence of SEQ ID NO:264; and
(b) a light chain variable (V_{L}) region comprising:
(1) a V_{L} CDR1 having the amino acid sequence of SEQ ID NO:265;
(2) a V_{L} CDR2 having the amino acid sequence of SEQ ID NO:238; and
(3) a V_{L} CDR3 having the amino acid sequence of SEQ ID NO:125.

185. The antibody of claim 182, wherein the antibody comprises:
(a) a heavy chain variable (V_{H}) region comprising:
(1) a V_{H} CDR1 having the amino acid sequence of SEQ ID NO:240;
(2) a V_{H} CDR2 having the amino acid sequence of SEQ ID NO:239; and
(3) a V_{H} CDR3 having the amino acid sequence of SEQ ID NO:267; and
(b) a light chain variable (V_{L}) region comprising:
(1) a V_{L} CDR1 having the amino acid sequence of SEQ ID NO:268;
(2) a V_{L} CDR2 having the amino acid sequence of SEQ ID NO:83; and
(3) a V_{L} CDR3 having the amino acid sequence of SEQ ID NO:269.

186. The antibody of claim 182, wherein the antibody comprises:
(a) a heavy chain variable (V_{H}) region comprising:
(1) a V_{H} CDR1 having the amino acid sequence of SEQ ID NO:244;
(2) a V_{H} CDR2 having the amino acid sequence of SEQ ID NO:270; and
(3) a V_{H} CDR3 having the amino acid sequence of SEQ ID NO:271; and
(b) a light chain variable (V_{L}) region comprising:
(1) a V_{L} CDR1 having the amino acid sequence of SEQ ID NO:247;
(2) a V_{L} CDR2 having the amino acid sequence of SEQ ID NO:248; and
(3) a V_{L} CDR3 having the amino acid sequence of SEQ ID NO:272.

187. The antibody of claim 182, wherein the antibody comprises:
(a) a heavy chain variable (V_{H}) region comprising:
(1) a V_{H} CDR1 having the amino acid sequence of SEQ ID NO:250;
(2) a V_{H} CDR2 having the amino acid sequence of SEQ ID NO:273; and
(3) a V_{H} CDR3 having the amino acid sequence of SEQ ID NO:264; and
(b) a light chain variable (V_{L}) region comprising:
(1) a V_{L} CDR1 having the amino acid sequence of SEQ ID NO:265;
(2) a V_{L} CDR2 having the amino acid sequence of SEQ ID NO:238; and
(3) a V_{L} CDR3 having the amino acid sequence of SEQ ID NO:125.

188. The antibody of claim 83, wherein the antibody comprises:
(a) a heavy chain variable (V_{H}) region comprising:
(1) a V_{H} CDR1 having an amino acid sequence of selected from the group consisting of SEQ ID NO:112, 274, 266, 277, and 279;
(2) a V_{H} CDR2 having an amino acid sequence of selected from the group consisting of SEQ ID NO:117, 275, 239, 278, and 280; and
(3) a V_{H} CDR3 having an amino acid sequence of SEQ ID NO:121 or 276.

189. The antibody of claim 188, wherein the antibody comprises:
(a) a heavy chain variable (V_{H}) region comprising:
(1) a V_{H} CDR1 having the amino acid sequence of SEQ ID NO:112;
(2) a V_{H} CDR2 having the amino acid sequence of SEQ ID NO:117; and
(3) a V_{H} CDR3 having the amino acid sequence of SEQ ID NO:121.

190. The antibody of claim 188, wherein the antibody comprises:
(a) a heavy chain variable (V_{H}) region comprising:
(1) a V_{H} CDR1 having the amino acid sequence of SEQ ID NO:274;
(2) a V_{H} CDR2 having the amino acid sequence of SEQ ID NO:275; and
(3) a V_{H} CDR3 having the amino acid sequence of SEQ ID NO:276.

191. The antibody of claim 188, wherein the antibody comprises:
(a) a heavy chain variable (V_{H}) region comprising:
(1) a V_{H} CDR1 having the amino acid sequence of SEQ ID NO:266;
(2) a V_{H} CDR2 having the amino acid sequence of SEQ ID NO:239; and
(3) a V_{H} CDR3 having the amino acid sequence of SEQ ID NO:276.

192. The antibody of claim 188, wherein the antibody comprises:
(a) a heavy chain variable (V_{H}) region comprising:
(1) a V_{H} CDR1 having the amino acid sequence of SEQ ID NO:277;
(2) a V_{H} CDR2 having the amino acid sequence of SEQ ID NO:278; and
(3) a V_{H} CDR3 having the amino acid sequence of SEQ ID NO:121.

193. The antibody of claim 188, wherein the antibody comprises:
(a) a heavy chain variable (V_{H}) region comprising:
(1) a V_{H} CDR1 having the amino acid sequence of SEQ ID NO:279;
(2) a V_{H} CDR2 having the amino acid sequence of SEQ ID NO:280; and
(3) a V_{H} CDR3 having the amino acid sequence of SEQ ID NO:276.

194. The antibody of claim 83, wherein the antibody comprises:
(a) a heavy chain variable (V_{H}) region comprising:
(1) a V_{H} CDR1 having an amino acid sequence of selected from the group consisting of SEQ ID NO:126, 281, 285, 289, and 294;
(2) a V_{H} CDR2 having an amino acid sequence of selected from the group consisting of SEQ ID NO:128, 282, 162, 290, and 295; and
(3) a V_{H} CDR3 having an amino acid sequence of selected from the group consisting of SEQ ID NO:130, 283, 286, and 291;
and
(b) a light chain variable (V_{L}) region comprising:
(1) a V_{L} CDR1 having an amino acid sequence of selected from the group consisting of SEQ ID NO:88, 284, 287, 292, and 284;
(2) a V_{L} CDR2 having an amino acid sequence of selected from the group consisting of SEQ ID NO:71, 160, and 170; and
(3) a V_{L} CDR3 having an amino acid sequence of selected from the group consisting of SEQ ID NO:90, 288 and 293.

195. The antibody of claim 194, wherein the antibody comprises:
(a) a heavy chain variable (V_{H}) region comprising:
(1) a V_{H} CDR1 having the amino acid sequence of SEQ ID NO:126;
(2) a V_{H} CDR2 having the amino acid sequence of SEQ ID NO:128; and
(3) a V_{H} CDR3 having the amino acid sequence of SEQ ID NO:130; and
(b) a light chain variable (V_{L}) region comprising:
(1) a V_{L} CDR1 having the amino acid sequence of SEQ ID NO:88;
(2) a V_{L} CDR2 having the amino acid sequence of SEQ ID NO:71; and
(3) a V_{L} CDR3 having the amino acid sequence of SEQ ID NO:90.

196. The antibody of claim 194, wherein the antibody comprises:
(a) a heavy chain variable (V_{H}) region comprising:
(1) a V_{H} CDR1 having the amino acid sequence of SEQ ID NO:281;
(2) a V_{H} CDR2 having the amino acid sequence of SEQ ID NO:282; and
(3) a V_{H} CDR3 having the amino acid sequence of SEQ ID NO:283; and
(b) a light chain variable (V_{L}) region comprising:
(1) a V_{L} CDR1 having the amino acid sequence of SEQ ID NO:284;
(2) a V_{L} CDR2 having the amino acid sequence of SEQ ID NO:160; and
(3) a V_{L} CDR3 having the amino acid sequence of SEQ ID NO:90.

197. The antibody of claim 194, wherein the antibody comprises:
(a) a heavy chain variable (V_{H}) region comprising:
(1) a V_{H} CDR1 having the amino acid sequence of SEQ ID NO:285;
(2) a V_{H} CDR2 having the amino acid sequence of SEQ ID NO:162; and
(3) a V_{H} CDR3 having the amino acid sequence of SEQ ID NO:286; and
(b) a light chain variable (V_{L}) region comprising:
(1) a V_{L} CDR1 having the amino acid sequence of SEQ ID NO:287;
(2) a V_{L} CDR2 having the amino acid sequence of SEQ ID NO:71; and
(3) a V_{L} CDR3 having the amino acid sequence of SEQ ID NO:288.

198. The antibody of claim 194, wherein the antibody comprises:
(a) a heavy chain variable (V_{H}) region comprising:
(1) a V_{H} CDR1 having the amino acid sequence of SEQ ID NO:289;
(2) a V_{H} CDR2 having the amino acid sequence of SEQ ID NO:290; and
(3) a V_{H} CDR3 having the amino acid sequence of SEQ ID NO:291; and
(b) a light chain variable (V_{L}) region comprising:
(1) a V_{L} CDR1 having the amino acid sequence of SEQ ID NO:292;
(2) a V_{L} CDR2 having the amino acid sequence of SEQ ID NO:170; and
(3) a V_{L} CDR3 having the amino acid sequence of SEQ ID NO:293.

199. The antibody of claim 194, wherein the antibody comprises:
(a) a heavy chain variable (V_{H}) region comprising:
(1) a V_{H} CDR1 having the amino acid sequence of SEQ ID NO:294;
(2) a V_{H} CDR2 having the amino acid sequence of SEQ ID NO:295; and
(3) a V_{H} CDR3 having the amino acid sequence of SEQ ID NO:283; and
(b) a light chain variable (V_{L}) region comprising:
(1) a V_{L} CDR1 having the amino acid sequence of SEQ ID NO:284;
(2) a V_{L} CDR2 having the amino acid sequence of SEQ ID NO:160; and
(3) a V_{L} CDR3 having the amino acid sequence of SEQ ID NO:90.

200. The antibody of claim 83, wherein the antibody comprises:
(a) a heavy chain variable (V_{H}) region comprising:
(1) a V_{H} CDR1 having an amino acid sequence of selected from the group consisting of SEQ ID NO:126, 281, 285, 289, and 294;
(2) a V_{H} CDR2 having an amino acid sequence of selected from the group consisting of SEQ ID NO:128, 282, 162, 290, and 295; and
(3) a V_{H} CDR3 having an amino acid sequence of selected from the group consisting of SEQ ID NO:130, 283, 286, and 291;
and
(b) a light chain variable (V_{L}) region comprising:
(1) a V_{L} CDR1 having an amino acid sequence of selected from the group consisting of SEQ ID NO:88, 284, 287, and 292;
(2) a V_{L} CDR2 having an amino acid sequence of selected from the group consisting of SEQ ID NO:71, 160, and 170; and
(3) a V_{L} CDR3 having an amino acid sequence of selected from the group consisting of SEQ ID NO:90, 288 and 293.

201. The antibody of claim 200, wherein the antibody comprises:
(a) a heavy chain variable (V_{H}) region comprising:
(1) a V_{H} CDR1 having the amino acid sequence of SEQ ID NO:126;
(2) a V_{H} CDR2 having the amino acid sequence of SEQ ID NO:128; and
(3) a V_{H} CDR3 having the amino acid sequence of SEQ ID NO:130;
(b) a light chain variable (V_{L}) region comprising:
(1) a V_{L} CDR1 having the amino acid sequence of SEQ ID NO:88;
(2) a V_{L} CDR2 having the amino acid sequence of SEQ ID NO:71; and
(3) a V_{L} CDR3 having the amino acid sequence of SEQ ID NO:90.

202. The antibody of claim 200, wherein the antibody comprises:
(a) a heavy chain variable (V_{H}) region comprising:
(1) a V_{H} CDR1 having the amino acid sequence of SEQ ID NO:281;
(2) a V_{H} CDR2 having the amino acid sequence of SEQ ID NO:282; and
(3) a V_{H} CDR3 having the amino acid sequence of SEQ ID NO:283; and
(b) a light chain variable (V_{L}) region comprising:
(1) a V_{L} CDR1 having the amino acid sequence of SEQ ID NO:284;
(2) a V_{L} CDR2 having the amino acid sequence of SEQ ID NO:160; and
(3) a V_{L} CDR3 having the amino acid sequence of SEQ ID NO:90.

203. The antibody of claim 200, wherein the antibody comprises:
(a) a heavy chain variable (V_{H}) region comprising:
(1) a V_{H} CDR1 having the amino acid sequence of SEQ ID NO:285;
(2) a V_{H} CDR2 having the amino acid sequence of SEQ ID NO:162; and
(3) a V_{H} CDR3 having the amino acid sequence of SEQ ID NO:286; and
(b) a light chain variable (V_{L}) region comprising:
(1) a V_{L} CDR1 having the amino acid sequence of SEQ ID NO:287;
(2) a V_{L} CDR2 having the amino acid sequence of SEQ ID NO:71; and
(3) a V_{L} CDR3 having the amino acid sequence of SEQ ID NO:288.

204. The antibody of claim 200, wherein the antibody comprises:
(a) a heavy chain variable (V_{H}) region comprising:
(1) a V_{H} CDR1 having the amino acid sequence of SEQ ID NO:289;
(2) a V_{H} CDR2 having the amino acid sequence of SEQ ID NO:290; and
(3) a V_{H} CDR3 having the amino acid sequence of SEQ ID NO:291; and
(b) a light chain variable (V_{L}) region comprising:
(1) a V_{L} CDR1 having the amino acid sequence of SEQ ID NO:292;
(2) a V_{L} CDR2 having the amino acid sequence of SEQ ID NO:170; and
(3) a V_{L} CDR3 having the amino acid sequence of SEQ ID NO:293.

205. The antibody of claim 200, wherein the antibody comprises:
(a) a heavy chain variable (V_{H}) region comprising:
(1) a V_{H} CDR1 having the amino acid sequence of SEQ ID NO:294;
(2) a V_{H} CDR2 having the amino acid sequence of SEQ ID NO:295; and
(3) a V_{H} CDR3 having the amino acid sequence of SEQ ID NO:283; and
(b) a light chain variable (V_{L}) region comprising:
(1) a V_{L} CDR1 having the amino acid sequence of SEQ ID NO:284;
(2) a V_{L} CDR2 having the amino acid sequence of SEQ ID NO:160; and
(3) a V_{L} CDR3 having the amino acid sequence of SEQ ID NO:90.

206. The antibody of claim 83, wherein the antibody comprises:
(a) a heavy chain variable (V_{H}) region comprising:
(1) a V_{H} CDR1 having an amino acid sequence of selected from the group consisting of SEQ ID NO:126, 281, 285, 289, and 294;
(2) a V_{H} CDR2 having an amino acid sequence of selected from the group consisting of SEQ ID NO:128, 282, 162, 290, and 295; and
(3) a V_{H} CDR3 having an amino acid sequence of selected from the group consisting of SEQ ID NO:77, 296, 286, and 291.

207. The antibody of claim 206, wherein the antibody comprises:
(a) a heavy chain variable (V_{H}) region comprising:
(1) a V_{H} CDR1 having the amino acid sequence of SEQ ID NO:126;
(2) a V_{H} CDR2 having the amino acid sequence of SEQ ID NO:128; and
(3) a V_{H} CDR3 having the amino acid sequence of SEQ ID NO:77.

208. The antibody of claim 206, wherein the antibody comprises:
(a) a heavy chain variable (V_{H}) region comprising:
(1) a V_{H} CDR1 having the amino acid sequence of SEQ ID NO:281;
(2) a V_{H} CDR2 having the amino acid sequence of SEQ ID NO:282; and
(3) a V_{H} CDR3 having the amino acid sequence of SEQ ID NO:296.

209. The antibody of claim 206, wherein the antibody comprises:
(a) a heavy chain variable (V_{H}) region comprising:
(1) a V_{H} CDR1 having the amino acid sequence of SEQ ID NO:285;
(2) a V_{H} CDR2 having the amino acid sequence of SEQ ID NO:162; and
(3) a V_{H} CDR3 having the amino acid sequence of SEQ ID NO:286.

210. The antibody of claim 206, wherein the antibody comprises:
(a) a heavy chain variable (V_{H}) region comprising:
(1) a V_{H} CDR1 having the amino acid sequence of SEQ ID NO:289;
(2) a V_{H} CDR2 having the amino acid sequence of SEQ ID NO:290; and
(3) a V_{H} CDR3 having the amino acid sequence of SEQ ID NO:291.

211. The antibody of claim 206, wherein the antibody comprises:
(a) a heavy chain variable (V_{H}) region comprising:
(1) a V_{H} CDR1 having the amino acid sequence of SEQ ID NO:294;
(2) a V_{H} CDR2 having the amino acid sequence of SEQ ID NO:295; and
(3) a V_{H} CDR3 having the amino acid sequence of SEQ ID NO:296.

212. The antibody of claim 83, wherein the antibody comprises:
(a) a heavy chain variable (V_{H}) region comprising:
(1) a V_{H} CDR1 having an amino acid sequence of selected from the group consisting of SEQ ID NO:127, 297, 285, 299, and 301;
(2) a V_{H} CDR2 having an amino acid sequence of selected from the group consisting of SEQ ID NO:129, 298, 162, 300, and 302; and
(3) a V_{H} CDR3 having an amino acid sequence of selected from the group consisting of SEQ ID NO:130, 283, 286, and 291.

213. The antibody of claim 212, wherein the antibody comprises:
(a) a heavy chain variable (V_{H}) region comprising:
(1) a V_{H} CDR1 having the amino acid sequence of SEQ ID NO:127;
(2) a V_{H} CDR2 having the amino acid sequence of SEQ ID NO:129; and
(3) a V_{H} CDR3 having the amino acid sequence of SEQ ID NO:130.

214. The antibody of claim 212, wherein the antibody comprises:
(a) a heavy chain variable (V_{H}) region comprising:
(1) a V_{H} CDR1 having the amino acid sequence of SEQ ID NO:297;
(2) a V_{H} CDR2 having the amino acid sequence of SEQ ID NO:298; and
(3) a V_{H} CDR3 having the amino acid sequence of SEQ ID NO:283.

215. The antibody of claim 212, wherein the antibody comprises:
(a) a heavy chain variable (V_{H}) region comprising:
(1) a V_{H} CDR1 having the amino acid sequence of SEQ ID NO:285;
(2) a V_{H} CDR2 having the amino acid sequence of SEQ ID NO:162; and
(3) a V_{H} CDR3 having the amino acid sequence of SEQ ID NO:286.

216. The antibody of claim 212, wherein the antibody comprises:
(a) a heavy chain variable (V_{H}) region comprising:
(1) a V_{H} CDR1 having the amino acid sequence of SEQ ID NO:299;
(2) a V_{H} CDR2 having the amino acid sequence of SEQ ID NO:300; and
(3) a V_{H} CDR3 having the amino acid sequence of SEQ ID NO:291.

217. The antibody of claim 212, wherein the antibody comprises:
(a) a heavy chain variable (V_{H}) region comprising:
(1) a V_{H} CDR1 having the amino acid sequence of SEQ ID NO:301;
(2) a V_{H} CDR2 having the amino acid sequence of SEQ ID NO:302; and
(3) a V_{H} CDR3 having the amino acid sequence of SEQ ID NO:283.

218. The antibody of claim 83, wherein the antibody comprises:
(a) a heavy chain variable (V_{H}) region comprising:
(1) a V_{H} CDR1 having an amino acid sequence of selected from the group consisting of SEQ ID NO:126, 281, 285, 289, and 294;
(2) a V_{H} CDR2 having an amino acid sequence of selected from the group consisting of SEQ ID NO:128, 282, 162, 290, and 295; and
(3) a V_{H} CDR3 having an amino acid sequence of selected from the group consisting of SEQ ID NO:130, 283, 286, and 291.

219. The antibody of claim 218, wherein the antibody comprises:
(a) a heavy chain variable (V_{H}) region comprising:
(1) a V_{H} CDR1 having the amino acid sequence of SEQ ID NO:126;
(2) a V_{H} CDR2 having the amino acid sequence of SEQ ID NO:128; and
(3) a V_{H} CDR3 having the amino acid sequence of SEQ ID NO:130.

220. The antibody of claim 218, wherein the antibody comprises:
(a) a heavy chain variable (V_{H}) region comprising:
(1) a V_{H} CDR1 having the amino acid sequence of SEQ ID NO:281;
(2) a V_{H} CDR2 having the amino acid sequence of SEQ ID NO:282; and
(3) a V_{H} CDR3 having the amino acid sequence of SEQ ID NO:283.

221. The antibody of claim 218, wherein the antibody comprises:
(a) a heavy chain variable (V_{H}) region comprising:
(1) a V_{H} CDR1 having the amino acid sequence of SEQ ID NO:285;
(2) a V_{H} CDR2 having the amino acid sequence of SEQ ID NO:162; and
(3) a V_{H} CDR3 having the amino acid sequence of SEQ ID NO:286.

222. The antibody of claim 218, wherein the antibody comprises:
(a) a heavy chain variable (V_{H}) region comprising:
(1) a V_{H} CDR1 having the amino acid sequence of SEQ ID NO:289;
(2) a V_{H} CDR2 having the amino acid sequence of SEQ ID NO:290; and
(3) a V_{H} CDR3 having the amino acid sequence of SEQ ID NO:291.

223. The antibody of claim 218, wherein the antibody comprises:
(a) a heavy chain variable (V_{H}) region comprising:
(1) a V_{H} CDR1 having the amino acid sequence of SEQ ID NO:294;
(2) a V_{H} CDR2 having the amino acid sequence of SEQ ID NO:295; and
(3) a V_{H} CDR3 having the amino acid sequence of SEQ ID NO:283.

224. The antibody of claim 83, wherein the antibody comprises:
(a) a heavy chain variable (V_{H}) region comprising:
(1) a V_{H} CDR1 having an amino acid sequence of selected from the group consisting of SEQ ID NO:91, 303, 307, 311, and 317;
(2) a V_{H} CDR2 having an amino acid sequence of selected from the group consisting of SEQ ID NO:92, 304, 308, 312, and 316; and
(3) a V_{H} CDR3 having an amino acid sequence of selected from the group consisting of SEQ ID NO:93, 305, 309, and 313; and
(b) a light chain variable (V_{L}) region comprising:
(1) a V_{L} CDR1 having an amino acid sequence of selected from the group consisting of SEQ ID NO:94, 237, 242, and 247;
(2) a V_{L} CDR2 having an amino acid sequence of selected from the group consisting of SEQ ID NO:83, 306, and 314; and
(3) a V_{L} CDR3 having an amino acid sequence of selected from the group consisting of SEQ ID NO:96, 310, and 315.

225. The antibody of claim 224, wherein the antibody comprises:
(a) a heavy chain variable (V_{H}) region comprising:
(1) a V_{H} CDR1 having the amino acid sequence of SEQ ID NO:91;
(2) a V_{H} CDR2 having the amino acid sequence of SEQ ID NO:92; and
(3) a V_{H} CDR3 having the amino acid sequence of SEQ ID NO:93; and
(b) a light chain variable (V_{L}) region comprising:
(1) a V_{L} CDR1 having the amino acid sequence of SEQ ID NO:94;
(2) a V_{L} CDR2 having the amino acid sequence of SEQ ID NO:83; and
(3) a V_{L} CDR3 having the amino acid sequence of SEQ ID NO:96.

226. The antibody of claim 224, wherein the antibody comprises:
(a) a heavy chain variable (V_{H}) region comprising:
(1) a V_{H} CDR1 having the amino acid sequence of SEQ ID NO:303;
(2) a V_{H} CDR2 having the amino acid sequence of SEQ ID NO:304; and
(3) a V_{H} CDR3 having the amino acid sequence of SEQ ID NO:305; and
(b) a light chain variable (V_{L}) region comprising:
(1) a V_{L} CDR1 having the amino acid sequence of SEQ ID NO:237;
(2) a V_{L} CDR2 having the amino acid sequence of SEQ ID NO:306; and
(3) a V_{L} CDR3 having the amino acid sequence of SEQ ID NO:96.

227. The antibody of claim 224, wherein the antibody comprises:
(a) a heavy chain variable (V_{H}) region comprising:
(1) a V_{H} CDR1 having the amino acid sequence of SEQ ID NO:307;
(2) a V_{H} CDR2 having the amino acid sequence of SEQ ID NO:308; and
(3) a V_{H} CDR3 having the amino acid sequence of SEQ ID NO:309; and
(b) a light chain variable (V_{L}) region comprising:
(1) a V_{L} CDR1 having the amino acid sequence of SEQ ID NO:242;
(2) a V_{L} CDR2 having the amino acid sequence of SEQ ID NO:83; and
(3) a V_{L} CDR3 having the amino acid sequence of SEQ ID NO:310.

228. The antibody of claim 224, wherein the antibody comprises:
(a) a heavy chain variable (V_{H}) region comprising:
(1) a V_{H} CDR1 having the amino acid sequence of SEQ ID NO:311;
(2) a V_{H} CDR2 having the amino acid sequence of SEQ ID NO:312; and
(3) a V_{H} CDR3 having the amino acid sequence of SEQ ID NO:313; and
(b) a light chain variable (V_{L}) region comprising:
(1) a V_{L} CDR1 having the amino acid sequence of SEQ ID NO:247;
(2) a V_{L} CDR2 having the amino acid sequence of SEQ ID NO:314; and
(3) a V_{L} CDR3 having the amino acid sequence of SEQ ID NO:315.

229. The antibody of claim 224, wherein the antibody comprises:
(a) a heavy chain variable (V_{H}) region comprising:
(1) a V_{H} CDR1 having the amino acid sequence of SEQ ID NO:317;
(2) a V_{H} CDR2 having the amino acid sequence of SEQ ID NO:316; and
(3) a V_{H} CDR3 having the amino acid sequence of SEQ ID NO:305; and
(b) a light chain variable (V_{L}) region comprising:
(1) a V_{L} CDR1 having the amino acid sequence of SEQ ID NO:237;
(2) a V_{L} CDR2 having the amino acid sequence of SEQ ID NO:306; and
(3) a V_{L} CDR3 having the amino acid sequence of SEQ ID NO:96.

230. An isolated antibody or functional fragment thereof which specifically binds to amino acid residues 1-31 of SEQ ID NO:1.

231. An isolated antibody or functional fragment thereof which specifically binds to amino acid residues 1-15 of SEQ ID NO:1 or amino acid residues 9-24 of SEQ ID NO:1.

232. The antibody or functional fragment of any one of claims 1-231, wherein the antibody is a monoclonal antibody.

233. The antibody or functional fragment of claim 232, wherein the monoclonal antibody is a humanized, human or chimeric antibody.

234. The antibody or functional fragment of any one of claims 1-231, wherein the fragment is an Fab, Fab′, F(ab′)₂, Fv, scFv, (scFv)₂, single chain antibody molecule, dual variable domain antibody, single variable domain antibody, linear antibody, V domain, or a multispecific antibody formed from antibody fragments.

235. A binding agent that binds to essentially the same epitope as an antibody of any one of claims 1-231.

236. The binding agent of claim 235, wherein the binding agent inhibits the growth of a tumor expressing C16orf54.

237. The binding agent of claim 235, which is an antibody or a functional fragment thereof.

238. The binding agent of claim 235, which is an anticalin, an adnectin, an affibody, a DARPin, a fynomer, an affitin, an affilin, an avimer, a cysteine-rich knottin peptide, or an engineered Kunitz-type inhibitor.

239. A binding agent capable of binding to C16orf54, wherein the antibody of any one of claims 1-231 displaces the binding agent in a competitive binding assay.

240. A binding agent capable of binding to C16orf54, wherein the binding agent displaces the antibody of any one of claims 1-231 in a competitive binding assay.

241. The binding agent of claim 239, wherein the binding agent is an antibody, or a functional fragment thereof.

242. The binding agent of claim 240, wherein the binding agent is an antibody, or a functional fragment thereof.

243. The antibody or functional fragment of any one of claims 1-231, 237, and 241-242, wherein the antibody or fragment is conjugated to a cytotoxic agent.

244. The antibody or functional fragment of claim 243, wherein the cytotoxic agent is selected from a chemotherapeutic agent, a drug, a growth inhibitory agent, a toxin, or a radioactive isotope.

245. The antibody or functional fragment of any one of claims 1-231, 237, and 241-242, wherein the antibody or fragment is conjugated to a detectable marker.

246. The antibody or functional fragment of claim 245, wherein the detectable marker is selected from a radioisotope, a metal chelator, an enzyme, a fluorescent compound, a bioluminescent compound and a chemiluminescent compound.

247. A transgenic animal that produces the monoclonal antibody of any one of claims 1-231.

248. A hybridoma that produces the monoclonal antibody of any one of claims 1-231.

249. A vector comprising a polynucleotide encoding the antibody or fragment thereof of any one of claims 1-231, 237, and 241-246.

250. A pharmaceutical composition that comprises the antibody or functional fragment of any one of claims 1-231, 237, and 241-246, and a pharmaceutically acceptable carrier.

251. A method of inhibiting growth of cancer cells that express C16orf54, the method comprising exposing the cells to the antibody or functional fragment of any one of claims 1-231, 237, and 241-246.

252. The method of claim 251, wherein the cancer cells are from a cancer selected from chronic lymphocytic leukemia, acute lymphocytic leukemia, acute myeloid leukemia, chronic myeloid leukemia, a lymphoma, a myeloma, breast cancer and pancreatic cancer.

253. A method for treating a cancer in a subject comprising administering to the subject the pharmaceutical composition of claim 250.

254. The method of claim 253, wherein the cancer is selected from chronic lymphocytic leukemia, acute lymphocytic leukemia, acute myeloid leukemia, chronic myeloid leukemia, a lymphoma, a myeloma, breast cancer and pancreatic cancer.

255. The method of claim 253, wherein the subject is administered one or more chemotherapeutic compound in combination with the antibody or functional fragment, wherein the chemotherapeutic compound is selected from chlorambucil, bendamustine hydrochloride, clophosphamide, fludurabine, pentostatin, cladribine, nelarabine, cytarabine, prednisone, prednisolone, methylprednisolone, dexamethasone, melphalan, lenalidomide, thalidomide, flavopiridol, oblimersen, ABT-263, doxorubicin, daunorubicin, idarubicin, mitoxentrone, methotrexate, clofarabine, imatinib mesylate, bosutinib, dasatinib, nilotinib, bortezomib, azacytidine, decitabine, midostaurin, sorafenib, AC220, arsenic trioxide, all-trans retinoic acid, vincristine sulfate, rituximab, ofatumumab, obinutuzumab, veltuzumab, ocrelizumab, lumiliximab, alemtuzumab and gemtuzumab ozogamicin.

256. The method of claim 255, wherein the one or more chemotherapeutic compound is selected from chlorambucil, bendamustine hydrochloride, clophosphamide, fludurabine, pentostatin, cladribine, prednisone, prednisolone, lenalidomide, flavopiridol, oblimersen, ABT-263, rituximab, ofatumumab, obinutuzumab, veltuzumab, ocrelizumab, lumiliximab, and alemtuzumab.

257. The method of claim 255, wherein the one or more chemotherapeutic compound is selected from cytarabine, lenalidomide, doxorubicin, daunorubicin, idarubicin, mitoxentrone, clofarabine, azacytidine, decitabine, midostaurin, sorafenib, AC220, arsenic trioxide, all-trans retinoic acid, vincristine sulfate, and gemtuzumab ozogamicin.

258. The method of claim 253 wherein the cancer is associated with increased expression of C16orf54 on the surface of a cell.

259. A method of detecting the presence of C16orf54 in a biological sample, comprising contacting the biological sample with an antibody of any one of claims 1-231, 237, and 241-246 under conditions permissive for binding of the antibody to C16orf54, and detecting whether a complex is formed between the antibody and C16orf54.

260. The method of claim 259, wherein the biological sample is from a mammal having or suspected of having a cancer selected from chronic lymphocytic leukemia, acute lymphocytic leukemia, acute myeloid leukemia, chronic myeloid leukemia, a lymphoma, a myeloma, breast cancer and pancreatic cancer.

261. A method of diagnosing a cancer associated with increased expression of C16orf54, comprising contacting a test cell with an antibody of any one of claims 1-231, 237, and 241-246; determining the level of expression of C16orf54 by detecting binding of the antibody to C16orf54; and comparing the level of expression of C16orf54 in the test cell with the level of expression of C16orf54 in a control cell, wherein a higher level of expression of C16orf54 in the test cell as compared to the control cell indicates the presence of a cancer associated with increased expression of C16orf54.

262. The method of claim 261 wherein the test cell is from a patient suspected of having a cancer selected from chronic lymphocytic leukemia, acute lymphocytic leukemia, acute myeloid leukemia, chronic myeloid leukemia, a lymphoma, a myeloma, breast cancer and pancreatic cancer.

263. The method of claim 262 wherein the method comprises determining the level of expression of C16orf54 on the surface of the test cell and comparing the level of expression of C16orf54 on the surface of the test cell with the level of expression of C16orf54 on the surface of the control cell.

264. The method of claim 263 wherein the test cell is a cancer cell and the control cell is a normal cell of the same tissue type.

265. The method of claim 263 wherein the test cell is a leukemia cell and the control cell is a bone marrow mononuclear cell or a peripheral blood mononuclear cell.

266. Use of the antibody or functional fragment of any one of claims 1-231, 237, and 241-246 in the manufacture of a medicament, wherein the medicament is for use in a method of inhibiting growth of cancer cells that express C16orf54, the method comprising exposing the cells to the antibody or functional fragment.

267. An antibody or functional fragment of any one of claims 1-231, 237, and 241-246 for use in inhibiting the growth of cancer cells that express C16orf54.

268. Use of the pharmaceutical composition of claim 250 in the manufacture of a medicament, wherein the medicament is for use in a method of treating cancer in a subject, the method comprising administering the pharmaceutical composition to the subject.

269. A pharmaceutical composition that comprises the antibody or functional fragment of any one of claims 1-231, 237, and 241-246, and a pharmaceutically acceptable carrier, for use in treating cancer in a subject.

270. Use of an antibody or functional fragment of any one of claims 1-231, 237, and 241-246 in the manufacture of a medicament, wherein the medicament is for use in a method for detecting the presence of C16orf54 in a biological sample, the method comprising contacting the biological sample with the antibody under conditions permissive for binding of the antibody to C16orf54, and detecting whether a complex is formed between the antibody and C16orf54.

271. An antibody or functional fragment of any one of claims 1-231, 237, and 241-246 for use in a method of detecting the presence of C16orf54 in a biological sample, the method comprising contacting the biological sample with the antibody under conditions permissive for binding of the antibody to C16orf54, and detecting whether a complex is formed between the antibody and C16orf54.

272. Use of an antibody or functional fragment of any one of claims 1-231, 237, and 241-246 in the manufacture of a medicament, wherein the medicament is for use in a method of diagnosing a cancer associated with increased expression of C16orf54, the method comprising determining the level of expression of C16orf54 by detecting binding of the antibody to C16orf54; and comparing the level of expression of C16orf54 in the test cell with the level of expression of C16orf54 in a control cell, wherein a higher level of expression of C16orf54 in the test cell as compared to the control cell indicates the presence of a cancer associated with increased expression of C16orf54.

273. An antibody or functional fragment of any one of claims 1-231, 237, and 241-246 for use in a method of diagnosing a cancer associated with increased expression of C16orf54, the method comprising determining the level of expression of C16orf54 by detecting binding of the antibody to C16orf54; and comparing the level of expression of C16orf54 in the test cell with the level of expression of C16orf54 in a control cell, wherein a higher level of expression of C16orf54 in the test cell as compared to the control cell indicates the presence of a cancer associated with increased expression of C16orf54.

274. The antibody or functional fragment of any one of claims 243-246, wherein the conjugated antibody or conjugated fragment comprises a linker.

275. The antibody or functional fragment of claim 274, wherein the antibody or functional fragment comprises a linker of the following formula:
(L¹)ₐ-(L²)_{b}-(L³)_{c}
wherein:
L¹, L² and L³ is independently a linker selected from the group consisting of —O—, —C(O)—, —S—, —S(O)—, —S(O)₂—, —NH—, —NCH₃—, —(CH₂)_{q}—, —NH(CH₂)₂NH—, —OC(O)—, —CO₂—, —NHCH₂CH₂C(O)—, —C(O)NHCH₂CH₂NH—, —NHCH₂C(O)—, —NHC(O)—, —C(O)NH—, —NCH₃C(O)—, —C(O)NCH₃—, —(CH2CH₂O)ₚ, —(CH2CH₂O)ₚCH₂CH₂—, —CH₂CH₂—(CH₂CH₂O)ₚ—, —OCH(CH₂O—)₂, -(AA)ᵣ-, cyclopentanyl, cyclohexanyl, unsubstituted phenylenyl, and phenylenyl substituted by 1 or 2 substituents selected from the group consisting of halo, CF₃—, CF₃O—, CH₃O—, —C(O)OH, —C(O)OC₁₋₃ alkyl, —C(O)CH₃, —CN, —NH₂, —OH, —NHCH₃, —N(CH₃)₂, and C₁₋₃ alkyl.

276. The antibody or functional fragment of claim 274 or 275, wherein the linker comprises valine and/or citrulline.

277. The antibody or functional fragment of claim 274, wherein the cytotoxic agent is selected from the group consisting of a tubulin stabilizer, a tubulin destabilizer, a DNA alkylator, a DNA minor groove binder, a DNA intercalator, a topoisomerase I inhibitor, a topoisomerase II inhibitor, a gyrase inhibitor, a protein synthesis inhibitor, a proteosome inhibitor, and an anti-metabolite.

278. The antibody or functional fragment of claim 274, wherein the cytotoxic agent is selected from the group consisting of Actinomycin D, Amonafide, an auristatin, benzophenone, benzothiazole, a calicheamicin, Camptothecin, CC-1065 (NSC 298223), Cemadotin, Colchicine, Combretastatin A4, Dolastatin, Doxorubicin, Elinafide, Emtansine (DM1), Etoposide, KF-12347 (Leinamycin), a maytansinoid, Methotrexate, Mitoxantrone, Nocodazole, Proteosome Inhibitor 1 (PSI 1), Roridin A, T-2 Toxin (trichothecene analog), Taxol, a tubulysin, Velcade®, and Vincristine.

279. The antibody or functional fragment of claim 274, wherein the cytotoxic agent is an auristatin, a calicheamicin, a maytansinoid, or a tubulysin.

280. The antibody or functional fragment of claim 274, wherein the cytotoxic agent is monomethylauristatin E, monomethylauristatin F, calicheamicin γ, mertansine, tubulysin T3, or tubulysin T4.

281. The antibody or functional fragment of claim 274, wherein the cytotoxic agent is MMAE or MMAF.

282. An antibody-drug conjugate of the following formula (Ia) or (Ib): 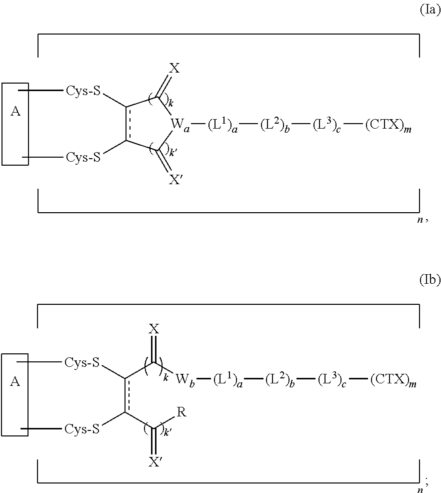
or a pharmaceutically acceptable salt thereof;
wherein:
A is an antibody or antibody fragment;
the two depicted cysteine residues are from an opened cysteine-cysteine disulfide bond in A;
each X and X′ is independently O, S, NH, or NR¹ wherein R¹ is C₁₋₆ alkyl;
Wa is ═N—, ═CH—, ═CHCH₂—, ═C(R²)—, or ═CHCH(R²)—; Wb is —NH—, —N(R¹)—, —CH₂—, —CH₂—NH—, —CH₂—N(R¹)—, —CH₂CH₂—, —CH(R²)—, or —CH₂CH(R²)—;
wherein R¹ and R² are independently C₁₋₆ alkyl;
CTX is a cytotoxic agent;
R is any chemical group; or R is absent;
each L¹, L² and L³ is independently a linker selected from the group consisting of —O—, —C(O)—, —S—, —S(O)—, —S(O)₂—, —NH—, —NCH₃—, —(CH₂)_{q}—, —NH(CH₂)₂NH—, —OC(O)—, —CO₂—, —NHCH₂CH₂C(O)—, —C(O)NHCH₂CH₂NH—, —NHCH₂C(O)—, —NHC(O)—, —C(O)NH—, —NCH₃C(O)—, —C(O)NCH₃—, —(CH₂CH₂O)ₚ, —(CH₂CH₂O)ₚCH₂CH₂—, —CH₂CH₂—(CH₂CH₂O)ₚ—, —OCH(CH₂O—)₂, -(AA)ᵣ-, cyclopentanyl, cyclohexanyl, unsubstituted phenylenyl, and phenylenyl substituted by 1 or 2 substituents selected from the group consisting of halo, CF₃—, CF₃O—, CH₃O—, —C(O)OH, —C(O)OC₁₋₃ alkyl, —C(O)CH₃, —CN, —NH₂, —OH, —NHCH₃, —N(CH₃)₂, and C₁₋₃ alkyl;
a, b and c are each independently an integer of 0, 1, 2 or 3, provided that at least one of a, b or c is 1;
each k and k′ is independently an integer of 0 or 1;
each p is independently an integer of 1 to 14;
each q is independently an integer from 1 to 12;
each AA is independently an amino acid;
each r is 1 to 12;
m is an integer of 1 to 4;
n is an integer of 1 to 4; and
the [symbol] bond represents a single or a double bond.

283. The antibody-drug conjugate of claim 282, wherein A is an anti-C16orf54 antibody.

284. The antibody-drug conjugate of claim 282, wherein A is an antibody or functional fragment of any one of claims 1-231, 237, and 241-246.

285. The antibody-drug conjugate of claim 282, wherein CTX is selected from the group consisting of a tubulin stabilizer, a tubulin destabilizer, a DNA alkylator, a DNA minor groove binder, a DNA intercalator, a topoisomerase I inhibitor, a topoisomerase II inhibitor, a gyrase inhibitor, a protein synthesis inhibitor, a proteosome inhibitor, and an anti-metabolite.

286. The antibody-drug conjugate of claim 282, wherein CTX is selected from the group consisting of Actinomycin D, Amonafide, an auristatin, benzophenone, benzothiazole, a calicheamicin, Camptothecin, CC-1065 (NSC 298223), Cemadotin, Colchicine, Combretastatin A4, Dolastatin, Doxorubicin, Elinafide, Emtansine (DM1), Etoposide, KF-12347 (Leinamycin), a maytansinoid, Methotrexate, Mitoxantrone, Nocodazole, Proteosome Inhibitor 1 (PSI 1), Roridin A, T-2 Toxin (trichothecene analog), Taxol, a tubulysin, Velcade®, and Vincristine.

287. The antibody-drug conjugate of claim 282, wherein CTX is an auristatin, a calicheamicin, a maytansinoid, or a tubulysin.

288. The antibody-drug conjugate of claim 282, wherein CTX is monomethylauristatin E, monomethylauristatin F, calicheamicin γ, mertansine, tubulysin T3, or tubulysin T4.

289. The antibody-drug conjugate of claim 282, wherein the cytotoxic agent is MMAE or MMAF.

290. An antibody-drug conjugate comprising:
(a) an antibody or functional fragment thereof that binds to C16orf54;
(b) optionally, a linker; and
(c) a cytotoxic agent.

291. The antibody-drug conjugate of claim 290, wherein the antibody-drug conjugate comprises a linker of the following formula:
(L¹)ₐ-(L²)_{b}-(L³)_{c}
wherein:
L¹, L² and L³ is independently a linker selected from the group consisting of —O—, —C(O)—, —S—, —S(O)—, —S(O)₂—, —NH—, —NCH₃—, —(CH₂)_{q}—, —NH(CH₂)₂NH—, —OC(O)—, —CO₂—, —NHCH₂CH₂C(O)—, —C(O)NHCH₂CH₂NH—, —NHCH₂C(O)—, —NHC(O)—, —C(O)NH—, —NCH₃C(O)—, —C(O)NCH₃—, —(CH2CH₂O)ₚ, —(CH2CH₂O)ₚCH₂CH₂—, —CH₂CH₂—(CH₂CH₂O)ₚ—, —OCH(CH₂O—)₂, -(AA)ᵣ-, cyclopentanyl, cyclohexanyl, unsubstituted phenylenyl, and phenylenyl substituted by 1 or 2 substituents selected from the group consisting of halo, CF₃—, CF₃O—, CH₃O—, —C(O)OH, —C(O)OC₁₋₃ alkyl, —C(O)CH₃, —CN, —NH₂, —OH, —NHCH₃, —N(CH₃)₂, and C₁₋₃ alkyl.

292. The antibody-drug conjugate of claim 290 or 291, wherein the linker comprises valine and/or citrulline.

293. The antibody-drug conjugate of claim 290, wherein the antibody or functional fragment is an antibody or functional fragment of any one of claims 1-231, 237, and 241-246.

294. The antibody-drug conjugate of claim 290, wherein the cytotoxic agent is selected from the group consisting of a tubulin stabilizer, a tubulin destabilizer, a DNA alkylator, a DNA minor groove binder, a DNA intercalator, a topoisomerase I inhibitor, a topoisomerase II inhibitor, a gyrase inhibitor, a protein synthesis inhibitor, a proteosome inhibitor, and an anti-metabolite.

295. The antibody-drug conjugate of claim 290, wherein the cytotoxic agent is selected from the group consisting of Actinomycin D, Amonafide, an auristatin, benzophenone, benzothiazole, a calicheamicin, Camptothecin, CC-1065 (NSC 298223), Cemadotin, Colchicine, Combretastatin A4, Dolastatin, Doxorubicin, Elinafide, Emtansine (DM1), Etoposide, KF-12347 (Leinamycin), a maytansinoid, Methotrexate, Mitoxantrone, Nocodazole, Proteosome Inhibitor 1 (PSI 1), Roridin A, T-2 Toxin (trichothecene analog), Taxol, a tubulysin, Velcade®, and Vincristine.

296. The antibody-drug conjugate of claim 290, wherein the cytotoxic agent is an auristatin, a calicheamicin, a maytansinoid, or a tubulysin.

297. The antibody-drug conjugate of claim 290, wherein the cytotoxic agent is monomethylauristatin E, monomethylauristatin F, calicheamicin γ, mertansine, tubulysin T3, or tubulysin T4.

298. The antibody-drug conjugate of claim 290, wherein the cytotoxic agent is MMAE or MMAF.

299. The antibody-drug conjugate of claim 290, wherein the antibody-drug conjugate is of the following formula: 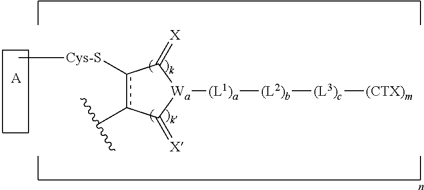
wherein:
A is an antibody or antibody fragment thereof;
the depicted cysteine residue is from an opened cysteine-cysteine disulfide bond in A;
each X and X′ is independently O, S, NH, or NR¹ wherein R¹ is C₁₋₆ alkyl;
Wa is ═N—, ═CH—, ═CHCH₂—, ═C(R²)—, or ═CHCH(R²)—; Wb is —NH—, —N(R¹)—, —CH₂—, —CH₂—NH—, —CH₂—N(R¹)—, —CH₂CH₂—, —CH(R²)—, or —CH₂CH(R²)—;
wherein R¹ and R² are independently C₁₋₆ alkyl;
CTX is a cytotoxic agent;
R is any chemical group; or R is absent;
each L¹, L² and L³ is independently a linker selected from the group consisting of —O—, —C(O)—, —S—, —S(O)—, —S(O)₂—, —NH—, —NCH₃—, —(CH₂)_{q}—, —NH(CH₂)₂NH—, —OC(O)—, —CO₂—, —NHCH₂CH₂C(O)—, —C(O)NHCH₂CH₂NH—, —NHCH₂C(O)—, —NHC(O)—, —C(O)NH—, —NCH₃C(O)—, —C(O)NCH₃—, —(CH₂CH₂O)ₚ, —(CH₂CH₂O)ₚCH₂CH₂—, —CH₂CH₂—(CH₂CH₂O)ₚ—, —OCH(CH₂O—)₂, -(AA)ᵣ-, cyclopentanyl, cyclohexanyl, unsubstituted phenylenyl, and phenylenyl substituted by 1 or 2 substituents selected from the group consisting of halo, CF₃—, CF₃O—, CH₃O—, —C(O)OH, —C(O)OC₁₋₃ alkyl, —C(O)CH₃, —CN, —NH₂, —OH, —NHCH₃, —N(CH₃)₂, and C₁₋₃ alkyl;
a, b and c are each independently an integer of 0, 1, 2 or 3, provided that at least one of a, b or c is 1;
each k and k′ is independently an integer of 0 or 1;
each p is independently an integer of 1 to 14;
each q is independently an integer from 1 to 12;
each AA is independently an amino acid;
each r is 1 to 12;
m is an integer of 1 to 4;
n is an integer of 1 to 4; and
the [symbol] bond represents a single or a double bond.
the [symbol] symbol represents a point of attachment to another chemical group or hydrogen.

300. The antibody-drug conjugate of claim 299, wherein the [symbol] symbol represents a point of attachment to a cysteine residue from an opened cysteine-cysteine disulfide bond in A.

301. A pharmaceutical composition that comprises the antibody or functional fragment of any one of claims 243-246 and 274-281, or the antibody-drug conjugate of any one of claims 282-300, and a pharmaceutically acceptable carrier.

302. A method of inhibiting growth of cancer cells that express 016orf54, the method comprising exposing the cells to the antibody or functional fragment of any one of claims 243-246 and 274-281, the antibody-drug conjugate of any one of claims 282-300, or the pharmaceutical composition of claim 301.

303. The method of claim 302, wherein the cancer cells are from a cancer selected from chronic lymphocytic leukemia, acute lymphocytic leukemia, acute myeloid leukemia, chronic myeloid leukemia, a lymphoma, a myeloma, breast cancer and pancreatic cancer.

304. A method for treating a cancer in a subject comprising administering to the subject the antibody or functional fragment of any one of claims 243-246 and 274-281, the antibody-drug conjugate of any one of claims 282-300, or the pharmaceutical composition of claim 301.

305. The method of claim 304, wherein the cancer is selected from chronic lymphocytic leukemia, acute lymphocytic leukemia, acute myeloid leukemia, chronic myeloid leukemia, a lymphoma, a myeloma, breast cancer and pancreatic cancer.

306. The method of claim 304, wherein the subject is administered one or more chemotherapeutic compound in combination with the antibody or functional fragment, wherein the chemotherapeutic compound is selected from chlorambucil, bendamustine hydrochloride, clophosphamide, fludurabine, pentostatin, cladribine, nelarabine, cytarabine, prednisone, prednisolone, methylprednisolone, dexamethasone, melphalan, lenalidomide, thalidomide, flavopiridol, oblimersen, ABT-263, doxorubicin, daunorubicin, idarubicin, mitoxentrone, methotrexate, clofarabine, imatinib mesylate, bosutinib, dasatinib, nilotinib, bortezomib, azacytidine, decitabine, midostaurin, sorafenib, AC220, arsenic trioxide, all-trans retinoic acid, vincristine sulfate, rituximab, ofatumumab, obinutuzumab, veltuzumab, ocrelizumab, lumiliximab, alemtuzumab and gemtuzumab ozogamicin.

307. The method of claim 306, wherein the one or more chemotherapeutic compound is selected from chlorambucil, bendamustine hydrochloride, clophosphamide, fludurabine, pentostatin, cladribine, prednisone, prednisolone, lenalidomide, flavopiridol, oblimersen, ABT-263, rituximab, ofatumumab, obinutuzumab, veltuzumab, ocrelizumab, lumiliximab, and alemtuzumab.

308. The method of claim 306, wherein the one or more chemotherapeutic compound is selected from cytarabine, lenalidomide, doxorubicin, daunorubicin, idarubicin, mitoxentrone, clofarabine, azacytidine, decitabine, midostaurin, sorafenib, AC220, arsenic trioxide, all-trans retinoic acid, vincristine sulfate, and gemtuzumab ozogamicin.

309. The method of claim 304 wherein the cancer is associated with increased expression of C16orf54 on the surface of a cell.

310. A method of detecting the presence of C16orf54 in a biological sample, comprising contacting the biological sample with the antibody or functional fragment of any one of claims 243-246 and 274-281, or the antibody-drug conjugate of any one of claims 282-300, under conditions permissive for binding of the antibody to C16orf54, and detecting whether a complex is formed between the antibody and C16orf54.

311. The method of claim 310, wherein the biological sample is from a mammal having or suspected of having a cancer selected from chronic lymphocytic leukemia, acute lymphocytic leukemia, acute myeloid leukemia, chronic myeloid leukemia, a lymphoma, a myeloma, breast cancer and pancreatic cancer.

312. A method of diagnosing a cancer associated with increased expression of C16orf54, comprising contacting a test cell with the antibody or functional fragment of any one of claims 243-246 and 274-281, or the antibody-drug conjugate of any one of claims 282-300; determining the level of expression of C16orf54 by detecting binding of the antibody to C16orf54; and comparing the level of expression of C16orf54 in the test cell with the level of expression of C16orf54 in a control cell, wherein a higher level of expression of C16orf54 in the test cell as compared to the control cell indicates the presence of a cancer associated with increased expression of C16orf54.

313. The method of claim 312, wherein the test cell is from a patient suspected of having a cancer selected from chronic lymphocytic leukemia, acute lymphocytic leukemia, acute myeloid leukemia, chronic myeloid leukemia, a lymphoma, a myeloma, breast cancer and pancreatic cancer.

314. The method of claim 312, wherein the method comprises determining the level of expression of C16orf54 on the surface of the test cell and comparing the level of expression of C16orf54 on the surface of the test cell with the level of expression of C16orf54 on the surface of the control cell.

315. The method of claim 314, wherein the test cell is a cancer cell and the control cell is a normal cell of the same tissue type.

316. The method of claim 314, wherein the test cell is a leukemia cell and the control cell is a bone marrow mononuclear cell or a peripheral blood mononuclear cell.

317. Use of the antibody or functional fragment of any one of claims 243-246 and 274-281, or the antibody-drug conjugate of any one of claims 282-300, in the manufacture of a medicament, wherein the medicament is for use in a method of inhibiting growth of cancer cells that express C16orf54, the method comprising exposing the cells to the antibody or functional fragment.

318. An antibody or functional fragment of any one of claims 243-246 and 274-281, or an antibody-drug conjugate of any one of claims 282-300, for use in inhibiting the growth of cancer cells that express C16orf54.

319. Use of the pharmaceutical composition of claim 301 in the manufacture of a medicament, wherein the medicament is for use in a method of treating cancer in a subject, the method comprising administering the pharmaceutical composition to the subject.

320. A pharmaceutical composition that comprises the antibody or functional fragment of any one of claims 243-246 and 274-281, or the antibody-drug conjugate of any one of claims 282-300, and a pharmaceutically acceptable carrier, for use in treating cancer in a subject.

321. Use of an antibody or functional fragment of any one of claims 243-246 and 274-281, or an antibody-drug conjugate of any one of claims 282-300, in the manufacture of a medicament, wherein the medicament is for use in a method for detecting the presence of C16orf54 in a biological sample, the method comprising contacting the biological sample with the antibody under conditions permissive for binding of the antibody to C16orf54, and detecting whether a complex is formed between the antibody and C16orf54.

322. An antibody or functional fragment of any one of claims 243-246 and 274-281, or an antibody-drug conjugate of any one of claims 282-300, for use in a method of detecting the presence of C16orf54 in a biological sample, the method comprising contacting the biological sample with the antibody under conditions permissive for binding of the antibody to C16orf54, and detecting whether a complex is formed between the antibody and C16orf54.

323. Use of an antibody or functional fragment of any one of claims 243-246 and 274-281, or an antibody-drug conjugate of any one of claims 282-300, in the manufacture of a medicament, wherein the medicament is for use in a method of diagnosing a cancer associated with increased expression of C16orf54, the method comprising determining the level of expression of C16orf54 by detecting binding of the antibody to C16orf54; and comparing the level of expression of C16orf54 in the test cell with the level of expression of C16orf54 in a control cell, wherein a higher level of expression of C16orf54 in the test cell as compared to the control cell indicates the presence of a cancer associated with increased expression of C16orf54.

324. An antibody or functional fragment of any one of claims 243-246 and 274-281, or an antibody-drug conjugate of any one of claims 282-300, for use in a method of diagnosing a cancer associated with increased expression of C16orf54, the method comprising determining the level of expression of C16orf54 by detecting binding of the antibody to C16orf54; and comparing the level of expression of C16orf54 in the test cell with the level of expression of C16orf54 in a control cell, wherein a higher level of expression of C16orf54 in the test cell as compared to the control cell indicates the presence of a cancer associated with increased expression of C16orf54.


SEQUENCE LISTING (319 sequences)

SEQ ID NO: 1 - protein, 224 aa, homo sapiens
  human C16orf54
MPLTPEPPSGRVEGPPAWEAAPWPSLPCGPCIPIMLVLATLAALFILTTAVLAERLFRRALRPDPSHRAPTLVWRPGGELWIEPMGTARERSEDWYGSAVPLLTDRAPEPPTQVGTLEARATAPPAPSAPNSAPSNLGPQTVLEVPARSTFWGPQPWEGRPPATGLVSWAEPEQRPEASVQFGSPQARRQRPGSPDPEWGLQPRVTLEQISAFWKREGRTSVGF

SEQ ID NO: 2 - DNA, 675 bp, homo sapiens
  human C16orf54
atgccgttgactccagagccgccctctgggcgcgtggaggggccccccgcatgggaagcagccccatggccctcactgccctgtgggccctgcatccccatcatgctggtcctggccaccctggctgcgctcttcatcctcaccaccgctgtgttggctgaacgcctgttccgccgtgctctccgcccagaccccagccaccgtgcacccaccctggtgtggcgcccaggaggagagctgtggattgagcccatgggcaccgcccgagagcgctctgaggactggtatggctctgcggtccccctgctgacagatcgggcccctgagcctcccacccaggtgggcactttggaggcccgagcaacagccccacctgccccctcagccccaaattctgctcccagcaacttgggcccccagaccgtactggaggtcccagcccggagcaccttctgggggccccagccctgggaggggaggccccccgccacaggcctggtgagctgggctgaacccgagcagaggccagaggccagcgtccagtttgggagcccccaggccaggaggcagcggccagggagcccggatcctgagtggggcctccagccacgggtcaccttggagcagatctcagctttctggaagcgtgaaggccggaccagtgtggggttctga

SEQ ID NO: 3 - DNA, 414 bp, Artificial Sequence
  R29-7-2A heavy chain variable region
atgctgttggggctgaagtgggttttctttgttgttttttatcaaggtgtgccttgtgaggtgcagcttgttgagtctggtggaggattgatacagcctaaagggtcattgaaactctcatgtgtagcctctggattctccttcaatacccacgccatgaactgggtccgtcaggctccaggaaagggtttggaatgggttgctcgcataagaagtaaaagtaataattatgcaagatattatgccgattcagtgaaagacaggttcaccatctccagagatgattcacaaagcattctctatctgcaaatgaacaacttgaaaactgaggacacagccatgtattactgtgtaaaacagggggacggggggtttgcttactggggccaagggaccctggtcactgtctctgca

SEQ ID NO: 4 - protein, 138 aa, Artificial Sequence
  R29-7-2A heavy chain variable region
MLLGLKWVFFVVFYQGVPCEVQLVESGGGLIQPKGSLKLSCVASGFSFNTHAMNWVRQAPGKGLEWVARIRSKSNNYARYYADSVKDRFTISRDDSQSILYLQMNNLKTEDTAMYYCVKQGDGGFAYWGQGTLVTVSA

SEQ ID NO: 5 - DNA, 399 bp, Artificial Sequence
  R29-7-2A light chain variable region
atggattcacaggcccaggttcttatgttactgctgctatgggtatctggtacctgtggggacattgtgatgtcacagtctccatcctccctagctgtgtcagttggagagaaggttactatgagctgcaagtccagtcagagccttttatatagtagcaatcaaaagaactacttggcctggtaccagcagagaccagggcagtctcctaaactactgatttactgggcatccgctagggaatctggggtccctgatcgcttcacaggcagtggatctgggacagatttcactctcaccatcagcagtgtgaaggctgaagacctggcagtttattactgtcagcaatattatagctatcctcccacgttcggtgctgggaccaaggtggagctgaaa

SEQ ID NO: 6 - protein, 133 aa, Artificial Sequence
  R29-7-2A light chain variable region
MDSQAQVLMLLLLWVSGTCGDIVMSQSPSSLAVSVGEKVTMSCKSSQSLLYSSNQKNYLAWYQQRPGQSPKLLIYWASARESGVPDRFTGSGSGTDFTLTISSVKAEDLAVYYCQQYYSYPPTFGAGTKVELK

SEQ ID NO: 7 - DNA, 417 bp, Artificial Sequence
  R29-7-1C heavy chain variable region
atgagagtgctgattcttttgtggctgttcacagcctttcctggtatcctgtctgatgtgcagcttcaggagtcgggacctggcctggtgaaaccttctcagtctctgtccctcacctgcactgtcactggctactcaatcaccagtgattatgcctggaactggatccggcagtttccaggaaacaaactggagtggatgggctacataagctacagtggtagtattaggtacaacccatctctcaaaagtcgaatctctatcactcgagacacatccaagaaccagttcttcctgcagttgaattctgtgactactgaggacacagccacatattactgtgcaagagagaagtatgataactattactatgctatggactactggggtcaaggaacctcagtcaccgtctcctca

SEQ ID NO: 8 - protein, 139 aa, Artificial Sequence
  R29-7-1C heavy chain variable region
MRVLILLWLFTAFPGILSDVQLQESGPGLVKPSQSLSLTCTVTGYSITSDYAWNWIRQFPGNKLEWMGYISYSGSIRYNPSLKSRISITRDTSKNQFFLQLNSVTTEDTATYYCAREKYDNYYYAMDYWGQGTSVTVSS

SEQ ID NO: 9 - DNA, 396 bp, Artificial Sequence
  R29-7-1C light chain variable region
atggattcacaggcccaggttcttatgttactgctgctatgggtatctggtacctgtggggacattgtaatgtcacagtctccatcctccctagctgtgtcagttggagagaaggttactctgagctgcaagtccagtcagaaccttttatatagtaccaatcaaaagaactacttggcctggtaccagcagaaaccagggcagtctcctaaactgctgatttactgggcatccactagggaatctggggtccctgatcgcttcacaggcagtggatctgggacagatttcactctcaccatcagcagtgtgaaggctgaggacctggcagtttattactgtcagcaatattatagctatcggacgttcggtggaggcaccaagctggaaatcaaa

SEQ ID NO: 10 - protein, 132 aa, Artificial Sequence
  R29-7-1C light chain variable region
MDSQAQVLMLLLLWVSGTCGDIVMSQSPSSLAVSVGEKVTLSCKSSQNLLYSTNQKNYLAWYQQKPGQSPKLLIYWASTRESGVPDRFTGSGSGTDFTLTISSVKAEDLAVYYCQQYYSYRTFGGGTKLEIK

SEQ ID NO: 11 - DNA, 417 bp, Artificial Sequence
  R29-67-7A heavy chain variable region
atgagagtgctgattcttttgtggctgttcacagcctttcctggtatcctgtctgatgtgcagcttcaggagtcgggacctggcctggtgaaaccttctcagtctctgtccctcacctgcactgtcactggctactcaatcaccagtgattatgcctggaactggatccggcagtttccaggaaacaaactggagtggatgggctacataaactacagtggtcgcactagatataacccatctctcaatagtcgcatctctctcactcgagacacatccacgaaccagttcttcctgcagttgaattctgtgactactgaggacacagccacatattactgtgcaagagagaactatgattacgagttttatgctatggactactggggtcaaggaacctcagtcaccgtctcctca

SEQ ID NO: 12 - protein, 139 aa, Artificial Sequence
  R29-67-7A heavy chain variable region
MRVLILLWLFTAFPGILSDVQLQESGPGLVKPSQSLSLTCTVTGYSITSDYAWNWIRQFPGNKLEWMGYINYSGRTRYNPSLNSRISLTRDTSTNQFFLQLNSVTTEDTATYYCARENYDYEFYAMDYWGQGTSVTVSS

SEQ ID NO: 13 - DNA, 396 bp, Artificial Sequence
  R29-67-7A light chain variable region
atggaatcacaggcccaggttcttatgttactgctgctatgggtatctggtacctgtggggacattgtgatgtcacagtctccatcctccctagctgtgtcagttggagagaaggttactatgagctgcaagtccagtcagaaccttttatatagtagcaatcaaaagaactacttggcctggtaccagcagaaaccagggcagtctcctaaactgctgatttactgggcatccactagggaatctggggtccctgatcgcttcacaggcagtggatctgggacagatttcactctcaccatcagcagtgtgaaggctgaagacctggcagtttattactgtcagcaatattatatctatcggacgttcggtggaggcaccaagctggaaatcaaa

SEQ ID NO: 14 - protein, 132 aa, Artificial Sequence
  R29-67-7A light chain variable region
MESQAQVLMLLLLWVSGTCGDIVMSQSPSSLAVSVGEKVTMSCKSSQNLLYSSNQKNYLAWYQQKPGQSPKLLIYWASTRESGVPDRFTGSGSGTDFTLTISSVKAEDLAVYYCQQYYIYRTFGGGTKLEIK

SEQ ID NO: 15 - DNA, 417 bp, Artificial Sequence
  R29-8-136C heavy chain variable region
atgagagtgctgattcttttgtggctgttcacagcctttcctggtatcctgtctgatgtgcagcttcaggagtcgggacctggcctggtgaaaccttctcagtctctgtccctcacctgcactgtcactggctactcaatcaccagtgattatgcctggaactggatccggcagtttccaggaaacaaactggagtggatgggctacataaactatagtggtagtagtaggtacaacccatctctcaaaagtcgaatctctatcactcgagacacatccaagaaccacttcttcctgcagttgaattctgtgactcctgaggacacagccacatatcattgtgcaagagagaggtacgacggggtttattatggtatggactactggggtcaaggaacctcagtcaccgtgtcctca

SEQ ID NO: 16 - protein, 139 aa, Artificial Sequence
  R29-8-136C heavy chain variable region
MRVLILLWLFTAFPGILSDVQLQESGPGLVKPSQSLSLTCTVTGYSITSDYAWNWIRQFPGNKLEWMGYINYSGSSRYNPSLKSRISITRDTSKNHFFLQLNSVTPEDTATYHCARERYDGVYYGMDYWGQGTSVTVSS

SEQ ID NO: 17 - DNA, 396 bp, Artificial Sequence
  R29-8-136C light chain variable region
atggattcacaggcccaggttcttatgtcactgctgctatgggtatctggtacctgtggggacattgtgatgtcacagtctccatcctccctagctgtgtcagttggagagaaggttactatgagctgcaagtccagtcagagccttttatatagtagcaatcaaaagaactacttggcctggtaccagcagaaaccagggcagtctcctaaactgctgatttactgggcatccactagggaatctggggtccctgatcgcttcacaggcagtggatctgggacagatttcactctcaccatcagcagtgtgaagactgaagacctggcagtttattactgtcagcaatattatagctatcggacgttcggtggaggcaccaagctggaaatcaaa

SEQ ID NO: 18 - protein, 132 aa, Artificial Sequence
  R29-8-136C light chain variable region
MDSQAQVLMSLLLWVSGTCGDIVMSQSPSSLAVSVGEKVTMSCKSSQSLLYSSNQKNYLAWYQQKPGQSPKLLIYWASTRESGVPDRFTGSGSGTDFTLTISSVKTEDLAVYYCQQYYSYRTFGGGTKLEIK

SEQ ID NO: 19 - DNA, 417 bp, Artificial Sequence
  R29-8-57B heavy chain variable region
atgagagtgctgattcttttgtggctgttcacagcctttcctggtatcctgtctgatgtgcagcttcaggagtcgggacctggcctggtgaaaccttctcagtctctgtccctcacctgcactgtcactggctactcaatcaccagtgattatgcctggaactggatccggcagtttccaggaaacaaactggagtggatgggctacataaactatagtggtagtactaggtacaacccatctctcaaaagtcgaatctctatcactcgagacacatccaagaaccacttcttcctgcagttgaattctgtgactcctgaggacacagccacatatcattgtgcaagagagaggtacgacggggtttattatggtatggactactggggtcaaggaacctcagtcaccgtgtcctca

SEQ ID NO: 20 - protein, 139 aa, Artificial Sequence
  R29-8-57B heavy chain variable region
MRVLILLWLFTAFPGILSDVQLQESGPGLVKPSQSLSLTCTVTGYSITSDYAWNWIRQFPGNKLEWMGYINYSGSTRYNPSLKSRISITRDTSKNHFFLQLNSVTPEDTATYHCARERYDGVYYGMDYWGQGTSVTVSS

SEQ ID NO: 21 - DNA, 396 bp, Artificial Sequence; identical to SEQ ID NO: 17
  R29-8-57B light chain variable region

SEQ ID NO: 22 - protein, 132 aa, Artificial Sequence; identical to SEQ ID NO: 18
  R29-8-57B light chain variable region

SEQ ID NO: 23 - DNA, 408 bp, Artificial Sequence
  R29-7-54C heavy chain variable region
atggctgtcctggggctgcttctctgcctggtgacgttcccaagctgtgtcctgtcccaggtgcagctgaaggagtcaggacctggcctggtggcgccctcacagagcctgtccatcacatgcactgtctcagggttctcattaaccgactatgccataagctggattcgccagcctccaggaaagggtctggagtggctgggagtaatatggggtggtggaagaacatactctaattcagctctcaaatccagactgagcatcagcaaggacaactccaggagccaaattttcttaaaaatgaacagtctgcaaactgatgacacagccatgtactactgtgccaaacatgaggaggtatcccgttttgcttactggggccaagggactctggtcactgtctctgta

SEQ ID NO: 24 - protein, 136 aa, Artificial Sequence
  R29-7-54C heavy chain variable region
MAVLGLLLCLVTFPSCVLSQVQLKESGPGLVAPSQSLSITCTVSGFSLTDYAISWIRQPPGKGLEWLGVIWGGGRTYSNSALKSRLSISKDNSRSQIFLKMNSLQTDDTAMYYCAKHEEVSRFAYWGQGTLVTVSV

SEQ ID NO: 25 - DNA, 393 bp, Artificial Sequence
  R29-7-54C light chain variable region
atgaagttgcctgttaggctgttggtgctgatgttctggattcctgcttccatcagtgatgttgtgctgacccaaactccactctccctgcctgtcagtcttggagatcaagcctccatctcttgcagatctagtcagagccttgtttatagtaatggaaacagctatttacattggtacctgcagaagccaggccagtctccaaagctcctgatctacaaagtttccaaccgattttctggggtcccagacaggttcagtggcagtggatcagggacagatttcacactcaagatcactagagtggaggctgaggatctgggagtttatttctgctctcaaagtacacatattccgctcacgttcggtgctgggaccaagctggagctgaaa

SEQ ID NO: 26 - protein, 131 aa, Artificial Sequence
  R29-7-54C light chain variable region
MKLPVRLLVLMFWIPASISDVVLTQTPLSLPVSLGDQASISCRSSQSLVYSNGNSYLHWYLQKPGQSPKLLIYKVSNRFSGVPDRFSGSGSGTDFTLKITRVEAEDLGVYFCSQSTHIPLTFGAGTKLELK

SEQ ID NO: 27 - DNA, 408 bp, Artificial Sequence; identical to SEQ ID NO: 23
  R29-7-53A heavy chain variable region

SEQ ID NO: 28 - protein, 136 aa, Artificial Sequence; identical to SEQ ID NO: 24
  R29-7-53A heavy chain variable region

SEQ ID NO: 29 - DNA, 393 bp, Artificial Sequence; identical to SEQ ID NO: 25
  R29-7-53A light chain variable region

SEQ ID NO: 30 - protein, 131 aa, Artificial Sequence; identical to SEQ ID NO: 26
  R29-7-53A light chain variable region

SEQ ID NO: 31 - DNA, 402 bp, Artificial Sequence
  R29-8-50C heavy chain variable region
atgaacttcgggttcagcttgattttccttgtccttgtttcaaaaggtgtccagtgtgaagtgaagctggtggagtctgggggaggcttagtgaagcctggagggtccctgaaattctcctgtgcagcctctggattcactttcagtagctatgccgtgtcttgggttcgccagactccagagaagaggctggagtgggtcgcatccattactagtggtggtagaacctactatccagacagtgtgaagggccgattcaccatctccagagataatgccaggagcatgttgtacctgcacatgagcagtctgaggtctgaggacacggccatgtattactgtacaagaggctgggacgagaatgacttatggggccaaggcaccactctcaccgtctcctca

SEQ ID NO: 32 - protein, 134 aa, Artificial Sequence
  R29-8-50C heavy chain variable region
MNFGFSLIFLVLVSKGVQCEVKLVESGGGLVKPGGSLKFSCAASGFTFSSYAVSWVRQTPEKRLEWVASITSGGRTYYPDSVKGRFTISRDNARSMLYLHMSSLRSEDTAMYYCTRGWDENDLWGQGTTLTVSS

SEQ ID NO: 33 - DNA, 393 bp, Artificial Sequence
  R29-8-50C light chain variable region
atgaagttgcctgttaggctgttggtgctgatgttctggattcctgcttccagcagtgatgttgtgatgacccaaactccactctccctgcctgtcagtcttggagatcaagcctccatctcttgcagatctagtcagagccttgtatacagtaatggaaacacctatttacattggtacctgcagaggccaggccagtctccacagctcctgatctacaaagtttccaaccgattttctggggtcccagacaggttcagtggcagtggatcagggacagatttcacactcaagatcagcagagtggaggctgaggatctgggagtttatttctgctctcaaactacacatgttccgtggacgttcggtggaggcaccaagctggaaatcaaa

SEQ ID NO: 34 - protein, 131 aa, Artificial Sequence
  R29-8-50C light chain variable region
MKLPVRLLVLMFWIPASSSDVVMTQTPLSLPVSLGDQASISCRSSQSLVYSNGNTYLHWYLQRPGQSPQLLIYKVSNRFSGVPDRFSGSGSGTDFTLKISRVEAEDLGVYFCSQTTHVPWTFGGGTKLEIK

SEQ ID NO: 35 - DNA, 402 bp, Artificial Sequence
  R29-8-19B heavy chain variable region
atgaacttcgggttcagcttgattttccttgtccttgtttcaaaaggtgtccagtgtgaagtgaagctggtggagtctgggggaggcttagtgaagcctggagggtccctgaaattctcctgtgcagcctctggattcactttcagtagctatgccgtgtcttgggttcgccagactccagagaagaggctggagtgggtcgcatccattactagtggtggtagaacctattatccagacagtgtgaagggccgattcaccatctccagagataatgccaggagcatgttgtatttgcacatgagcagtctgaggtctgaggacacggccatgtattactgtacaagaggctgggacgagaatgacttatggggccaaggcaccactctcaccgtctcctca

SEQ ID NO: 36 - protein, 134 aa, Artificial Sequence; identical to SEQ ID NO: 32
  R29-8-19B heavy chain variable region

SEQ ID NO: 37 - DNA, 393 bp, Artificial Sequence
  R29-8-19B light chain variable region
atgaagttgcctgttaggctgttggtgctgatgttctggattcctgcttccagaagtgatgttgtgatgacccaaactccactctccctgcctgtcagtcttggagatcaagcctccatctcttgcagatctagtcagagccttgtatacagtaatggaaacacctatttacattggtacctgcagaagccaggccagtctccacagctcctgatctacaaagtttccaaccgattttctggggtcccagacaggttcagtggcagtggatcagggacagatttcacactcaagatcagcagagtggaggctgaggatctgggagtttatttctgctctcaaactacacatgttccgtggacgttcggtggaggcaccaagctggaaatcaaa

SEQ ID NO: 38 - protein, 131 aa, Artificial Sequence
  R29-8-19B light chain variable region
MKLPVRLLVLMFWIPASRSDVVMTQTPLSLPVSLGDQASISCRSSQSLVYSNGNTYLHWYLQKPGQSPQLLIYKVSNRFSGVPDRFSGSGSGTDFTLKISRVEAEDLGVYFCSQTTHVPWTFGGGTKLEIK

SEQ ID NO: 39 - DNA, 402 bp, Artificial Sequence
  R29-8-58C heavy chain variable region
atgaacttcgggttcagcttgattttccttgtccttgtttcaaaaggtgtccagtgtgaagtgaagctggtggagtctgggggaggcttagtgaagcctggagggtccctgaaattctcctgtgcagcctctggattcactttcagtagctatgccgtgtcttgggttcgccagactccagagaagaggctggagtgggtcgcatccattactagtggtggtagaagctactatccggacagtgtgaagggccgattcaccatctccagagataatgctaggagcatgttgtacctgcacatgagcagtctgaggtctgaggacacggccatgtactactgtacaagaggctgggacgagaatgacttatggggccaaggcaccactctcaccgtctcctca

SEQ ID NO: 40 - protein, 134 aa, Artificial Sequence
  R29-8-58C heavy chain variable region
MNFGFSLIFLVLVSKGVQCEVKLVESGGGLVKPGGSLKFSCAASGFTFSSYAVSWVRQTPEKRLEWVASITSGGRSYYPDSVKGRFTISRDNARSMLYLHMSSLRSEDTAMYYCTRGWDENDLWGQGTTLTVSS

SEQ ID NO: 41 - DNA, 393 bp, Artificial Sequence
  R29-8-58C light chain variable region
atgaagttgcctgttaggctgttggtgctgatgttctggattcctgcttccagcagtgatgttgtgatgacccaaactccactctccctgtttgtcagtcttggagatcaagcctccatctcttgcagatctagtcagagccttctttacagtaatggaaacacctatttacattggtacctgcagaggccaggccagtctccacagctcctgatctacaaagtttccaaccgattttctggggtcccagacaggttcagtggcagtggatcagggacagatttcacactcaagatcagcagagtggaggctgaggatctgggagtttatttctgctctcaaactacacatgttccgtggacgttcggtggaggcaccaagctggaaatcaaa

SEQ ID NO: 42 - protein, 131 aa, Artificial Sequence
  R29-8-58C light chain variable region
MKLPVRLLVLMFWIPASSSDVVMTQTPLSLFVSLGDQASISCRSSQSLLYSNGNTYLHWYLQRPGQSPQLLIYKVSNRFSGVPDRFSGSGSGTDFTLKISRVEAEDLGVYFCSQTTHVPWTFGGGTKLEIK

SEQ ID NO: 43 - DNA, 402 bp, Artificial Sequence
  R29-8-9B heavy chain variable region
atgaactacgggttcagcttgattttccttgtccttgttttaaaaggtgtccagtgtgaagtgaagctggtggagtctgggggaggcgtagtgaagcctggagggtccctgaaactctcctgtgcagcctctggattcactttcagtagctatgccatgtcctggtttcgccagacttcagagatgagtctggagtgggtcgcatccattactggtggtggtggcacctactatccagacagtgtgaagggccgattcaccatctccagagatactgtcaggaacatcctgtacctgcaaatgagaagtctgaggtctggggacacggccatatattactgtgcaaggggctgggacgagaatgactactggggccaaggcaccactctcacagtctcctca

SEQ ID NO: 44 - protein, 134 aa, Artificial Sequence
  R29-8-9B heavy chain variable region
MNYGFSLIFLVLVLKGVQCEVKLVESGGGVVKPGGSLKLSCAASGFTFSSYAMSWFRQTSEMSLEWVASITGGGGTYYPDSVKGRFTISRDTVRNILYLQMRSLRSGDTAIYYCARGWDENDYWGQGTTLTVSS

SEQ ID NO: 45 - DNA, 393 bp, Artificial Sequence
  R29-8-9B light chain variable region
atgaagttgcctgttaggctgttggtgctgatgttctggattcctgcttccagaagtgatgttgtgatgacccaaattccattgtccctgcctgtcagtcttggagatcaagcctccatctcttgcagatctagtcagagccttgtatttagtaatggaaacacctatttacattggtactttcagaagccaggccagtctccaaagctcctgatctacaaagtttccaaccgattttctggggtcccagacaggttcagtggcagtggatcagggacagatttcatactcagaatcagcagagtggaggctgaggatctgggagtttatttctgctctcaaagtacacatgttccgtggacgttcggtggaggcaccaagctggaaatcaaa

SEQ ID NO: 46 - protein, 131 aa, Artificial Sequence
  R29-8-9B light chain variable region
MKLPVRLLVLMFWIPASRSDVVMTQIPLSLPVSLGDQASISCRSSQSLVFSNGNTYLHWYFQKPGQSPKLLIYKVSNRFSGVPDRFSGSGSGTDFILRISRVEAEDLGVYFCSQSTHVPWTFGGGTKLEIK

SEQ ID NO: 47 - DNA, 402 bp, Artificial Sequence
  R29-8-28C heavy chain variable region
atgaacttcgggttcagcttgattttccttgtccttgttttaaaaggtgtccagtctgaagtgaagctggtggagtctgggggaggcgtagtgaagcctggagggtccctgacactctcctgtgcagcctctggattcactttcagtaactatgccgtgtcttggtttcgccagactccggagaagaggctggagtgggtcgcatccattactagtggtggtagaacctactatccagacagtatgaagggccgattcaccatctccagagataatgccaggaacatcttgtatctgcaaatgagcagtctgaggtctgaggacacggccatatattactgtggaagaggctgggacgagaatgactactggggccaaggcaccactctcaccgtctcctca

SEQ ID NO: 48 - protein, 134 aa, Artificial Sequence
  R29-8-28C heavy chain variable region
MNFGFSLIFLVLVLKGVQSEVKLVESGGGVVKPGGSLTLSCAASGFTFSNYAVSWFRQTPEKRLEWVASITSGGRTYYPDSMKGRFTISRDNARNILYLQMSSLRSEDTAIYYCGRGWDENDYWGQGTTLTVSS

SEQ ID NO: 49 - DNA, 393 bp, Artificial Sequence
  R29-8-28C light chain variable region
atgaagttgcctgttaggctgttggtgctgatgttctggattcctgcttcctgcagtgatgttgtgatgacccaaactccactctccctgcctgtcagtcttggagatcaagcctccatctcttgcagatctagtcaaagccttgtatacagtaatggaaacacctatttacattggtacctgcagaagccaggccagtctccaaaactcctgatctacaaagtttccaaccgattttctggggtcccagacaggttcagtggcagtggatcagggacagatttcacactcaagatcaacagagtggaggctgaggatctgggagtttatttctgctctcaaactacacatgttccgtggacgttcggtggaggcaccaagctggaaatcaaa

SEQ ID NO: 50 - protein, 131 aa, Artificial Sequence
  R29-8-28C light chain variable region
MKLPVRLLVLMFWIPASCSDVVMTQTPLSLPVSLGDQASISCRSSQSLVYSNGNTYLHWYLQKPGQSPKLLIYKVSNRFSGVPDRFSGSGSGTDFTLKINRVEAEDLGVYFCSQTTHVPWTFGGGTKLEIK

SEQ ID NO: 51 - DNA, 402 bp, Artificial Sequence
  R29-8-120B heavy chain variable region
atgaacttcgggttcagcttgattttccttgtccttgttttaaaaggtgtccagtgtaaagtgaacctggtggagtctgggggaggcttagtgaaggctggagggtccctgaaactctcctgtgcagcctctggattcactttcagtatctatgccatgtcttggtttcgccagactccagagaagaggctggagtgggtcgcatccattactggtggtggtaccaatttctatccagacagtgtgaggggccgattcaccatctccagagataatgtcaggaacattctgtacctgcaaatgagcagtctgaggtctgaggacacggccatgtattactgtgcaagaggctgggacgagaatgactattggggccaaggcaccactctcacagtctcctca

SEQ ID NO: 52 - protein, 134 aa, Artificial Sequence
  R29-8-120B heavy chain variable region
MNFGFSLIFLVLVLKGVQCKVNLVESGGGLVKAGGSLKLSCAASGFTFSIYAMSWFRQTPEKRLEWVASITGGGTNFYPDSVRGRFTISRDNVRNILYLQMSSLRSEDTAMYYCARGWDENDYWGQGTTLTVSS

SEQ ID NO: 53 - DNA, 393 bp, Artificial Sequence
  R29-8-120B light chain variable region
atgaagttgcctgttaggctgttggtgctgatgttctggattccagcttccagcagtgatgttgtgatgacccaaactccactctccctgcctgtcagtcttggagatcaagcctccatctcttgcagatctagtcagagccttgtatatagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtggacgttcggtggaggcaccaagctggaaatcaaa

SEQ ID NO: 54 - protein, 131 aa, Artificial Sequence
  R29-8-120B light chain variable region
MKLPVRLLVLMFWIPASSSDVVMTQTPLSLPVSLGDQASISCRSSQSLVYSNGNTYLHWYLQKPGQSPKLLIYKVSNRFSGVPDRFSGSGSGTDFTLKISRVEAEDLGVYFCSQSTHVPWTFGGGTKLEIK

SEQ ID NO: 55 - DNA, 411 bp, Artificial Sequence
  R29-8-75B heavy chain variable region
atggactccaggctcaatttagttttccttgtccttattttaaaaggtgtccagtgtgatgtgcagctggtggagtctgggggaggcttagtgcagcctggagggtcccggaaactctcctgtgcagcctctggattcactttcagtaggtttggaatgcactgggttcgtcaggctccagagaaggggctggagtgggtcgcatacattagtagtggcagtagtaccatctactatgcagacacagtgaagggccgattcaccatctccagagacaatcccaagaacaccctgttcctgcaaatgaccagtctaaggtctgaggacacggccatgtattactgtgcaagagttgattacgacgtggcgcttgcttactggggccaagggactctggtcactgtctctgca

SEQ ID NO: 56 - protein, 137 aa, Artificial Sequence
  R29-8-75B heavy chain variable region
MDSRLNLVFLVLILKGVQCDVQLVESGGGLVQPGGSRKLSCAASGFTFSRFGMHWVRQAPEKGLEWVAYISSGSSTIYYADTVKGRFTISRDNPKNTLFLQMTSLRSEDTAMYYCARVDYDVALAYWGQGTLVTVSA

SEQ ID NO: 57 - DNA, 387 bp, Artificial Sequence
  R29-8-75B light chain variable region
atgaagttgcctgttaggctgttggtgctgatgttctggattcctgcttccagcagtggtgttttgatgacccaaactccactctccctgcctgtcagtcttggagatcaagcctccatctcttgcagatctagtcagagcattgtacataggaatggaaatacctatttagaatggtacctgcagaaaccaggccagtctccaaaactcctgatctacaaagtttccaaccgattttctggggtcccagacaggttcagtggcagtggatcagggacagatttcacactcaagatcagcagagtggaggctgaggatctgggagtttattactgctttcaaggttcacagtggacgttcggtggaggcaccaagctggaaatcaaa

SEQ ID NO: 58 - protein, 129 aa, Artificial Sequence
  R29-8-75B light chain variable region
MKLPVRLLVLMFWIPASSSGVLMTQTPLSLPVSLGDQASISCRSSQSIVHRNGNTYLEWYLQKPGQSPKLLIYKVSNRFSGVPDRFSGSGSGTDFTLKISRVEAEDLGVYYCFQGSQWTFGGGTKLEIK

SEQ ID NO: 59 - DNA, 402 bp, Artificial Sequence; identical to SEQ ID NO: 39
  R29-8-36C heavy chain variable region

SEQ ID NO: 60 - protein, 134 aa, Artificial Sequence; identical to SEQ ID NO: 40
  R29-8-36C heavy chain variable region

SEQ ID NO: 61 - DNA, 393 bp, Artificial Sequence; identical to SEQ ID NO: 41
  R29-8-36C light chain variable region

SEQ ID NO: 62 - protein, 131 aa, Artificial Sequence; identical to SEQ ID NO: 42
  R29-8-36C light chain variable region

SEQ ID NO: 63 - DNA, 402 bp, Artificial Sequence; identical to SEQ ID NO: 35
  R29-8-12A heavy chain variable region

SEQ ID NO: 64 - protein, 134 aa, Artificial Sequence; identical to SEQ ID NO: 32
  R29-8-12A heavy chain variable region

SEQ ID NO: 65 - DNA, 393 bp, Artificial Sequence; identical to SEQ ID NO: 37
  R29-8-12A light chain variable region

SEQ ID NO: 66 - protein, 131 aa, Artificial Sequence; identical to SEQ ID NO: 38
  R29-8-12A light chain variable region

SEQ ID NO: 67 - protein, 8 aa, Artificial Sequence
  VH CDR1 consensus sequence for Group I antibodies
GFTGSXYA

SEQ ID NO: 68 - protein, 8 aa, Artificial Sequence
  VH CDR2 consensus sequence for Group I antibodies
ITGXGGXX

SEQ ID NO: 69 - protein, 9 aa, Artificial Sequence
  VH CDR3 consensus sequence for Group I antibodies
XRGWDENDX

SEQ ID NO: 70 - protein, 11 aa, Artificial Sequence
  VL CDR1 consensus sequence for Group I antibodies
QSLXXSNGNTY

SEQ ID NO: 71 - protein, 3 aa, Artificial Sequence
  VL CDR2 consensus sequence for Group I antibodies
KVS

SEQ ID NO: 72 - protein, 9 aa, Artificial Sequence
  VL CDR3 consensus sequence for Group I antibodies
SQXTHVPWT

SEQ ID NO: 73 - protein, 8 aa, Artificial Sequence
  VH CDR1 consensus sequence for Group II antibodies
GFTFSRFG

SEQ ID NO: 74 - protein, 8 aa, Artificial Sequence
  VH CDR2 consensus sequence for Group II antibodies
ISSGSSTI

SEQ ID NO: 75 - protein, 11 aa, Artificial Sequence
  VH CDR3 consensus sequence for Group II antibodies
ARVDYDVALAY

SEQ ID NO: 76 - protein, 11 aa, Artificial Sequence
  VL CDR1 consensus sequence for Group II antibodies
QSIVHRNGNTY

SEQ ID NO: 77 - protein, 11 aa, Artificial Sequence
  VH CDR3 sequence for antibody R29-7A-38C
AKHEEVSRFAH

SEQ ID NO: 78 - protein, 7 aa, Artificial Sequence
  VH CDR3 consensus sequence for Group II antibodies
FQGSQWT

SEQ ID NO: 79 - protein, 9 aa, Artificial Sequence
  VH CDR1 consensus sequence for Group III antibodies
GYSITSXYA

SEQ ID NO: 80 - protein, 7 aa, Artificial Sequence
  VH CDR2 consensus sequence for Group III antibodies
IXYSGXX

SEQ ID NO: 81 - protein, 14 aa, Artificial Sequence
  VH CDR3 consensus sequence for Group III antibodies
AREXYDXXXYXMDY

SEQ ID NO: 82 - protein, 12 aa, Artificial Sequence
  VL CDR1 consensus sequence for Group III antibodies
QXLLYSXNQKNY

SEQ ID NO: 83 - protein, 3 aa, Artificial Sequence
  VL CDR2 consensus sequence for Group III antibodies
WAS

SEQ ID NO: 84 - protein, 8 aa, Artificial Sequence
  VL CDR3 consensus sequence for Group III antibodies
QQYYXYRT

SEQ ID NO: 85 - protein, 8 aa, Artificial Sequence
  VH CDR1 consensus sequence for Group IV antibodies
GFSLTDYX

SEQ ID NO: 86 - protein, 7 aa, Artificial Sequence
  VH CDR2 consensus sequence for Group IV antibodies
IWGGGXT

SEQ ID NO: 87 - protein, 11 aa, Artificial Sequence
  VH CDR3 consensus sequence for Group IV antibodies
AKHEEVSRFAX

SEQ ID NO: 88 - protein, 11 aa, Artificial Sequence
  VL CDR1 consensus sequence for Group IV antibodies
QSLVYSNGNSY

SEQ ID NO: 89 - protein, 8 aa, Artificial Sequence
  VH CDR1 sequence of antibody R29-8-28C
GFTFSNYA

SEQ ID NO: 90 - protein, 9 aa, Artificial Sequence
  VL CDR3 consensus sequence for Group IV antibodies
SQSTHIPLT

SEQ ID NO: 91 - protein, 8 aa, Artificial Sequence
  VH CDR1 sequence for Group IV antibody R29-7-2A
GFSFNTHA

SEQ ID NO: 92 - protein, 10 aa, Artificial Sequence
  VH CDR2 sequence for Group IV antibody R29-7-2A
IRSKSNNYAR

SEQ ID NO: 93 - protein, 10 aa, Artificial Sequence
  VH CDR3 sequence for Group IV antibody R29-7-2A
VKQGDGGFAY

SEQ ID NO: 94 - protein, 12 aa, Artificial Sequence
  VL CDR1 sequence for Group IV antibody R29-7-2A
QSLLYSSNQKNY

SEQ ID NO: 95 - protein, 8 aa, Artificial Sequence
  VH CDR1 sequence for Group I antibody R29-8-120B
GFTFSIYA

SEQ ID NO: 96 - protein, 9 aa, Artificial Sequence
  VH CDR1 sequence for Group I antibody R29-8-18B
QQYYSYPPT

SEQ ID NO: 97 - protein, 7 aa, Artificial Sequence
  VH CDR2 sequence for Group I antibody R29-8-9B
ITGGGGT

SEQ ID NO: 98 - protein, 7 aa, Artificial Sequence
  VH CDR2 sequence for Group I antibody R29-8-30A
ITGGGST

SEQ ID NO: 99 - protein, 7 aa, Artificial Sequence
  VH CDR2 sequence for Group I antibody R29-8-120B
ITGGGTN

SEQ ID NO: 100 - protein, 7 aa, Artificial Sequence
  VH CDR2 sequence for Group I antibody R29-8-18B
ITSGGST

SEQ ID NO: 101 - protein, 7 aa, Artificial Sequence
  VH CDR2 sequence for Group I antibody R29-8-28C
ITSGGRT

SEQ ID NO: 102 - protein, 7 aa, Artificial Sequence
  VH CDR2 sequence for Group I antibody R29-8-36C
ITSGGRS

SEQ ID NO: 103 - protein, 9 aa, Artificial Sequence
  VH CDR3 sequence for Group I antibody R29-8-9B
ARGWDENDY

SEQ ID NO: 104 - protein, 9 aa, Artificial Sequence
  VH CDR3 sequence for Group I antibody R29-8-28C
GRGWDENDY

SEQ ID NO: 105 - protein, 9 aa, Artificial Sequence
  VH CDR3 sequence for Group I antibody R29-8-19B
TRGWDENDL

SEQ ID NO: 106 - protein, 11 aa, Artificial Sequence
  VL CDR1 sequence for Group I antibody R29-8-9B
QSLVFSNGNTY

SEQ ID NO: 107 - protein, 11 aa, Artificial Sequence
  VL CDR1 sequence for Group I antibody R29-8-120B
QSLVYSNGNTY

SEQ ID NO: 108 - protein, 11 aa, Artificial Sequence
  VL CDR1 sequence for Group I antibody R29-8-36C
QSLLYSNGNTY

SEQ ID NO: 109 - protein, 9 aa, Artificial Sequence
  VL CDR3 sequence for Group I antibody R29-8-9B
SQSTHVPWT

SEQ ID NO: 110 - protein, 9 aa, Artificial Sequence
  VL CDR3 sequence for Group I antibody R29-8-28C
SQTTHVPWT

SEQ ID NO: 111 - protein, 9 aa, Artificial Sequence
  VH CDR1 sequence for Group III antibody R29-8-57B
GYSITSDYA

SEQ ID NO: 112 - protein, 9 aa, Artificial Sequence
  VH CDR1 sequence for Group III antibody R29-67-4A
GYSITSVYA

SEQ ID NO: 113 - protein, 7 aa, Artificial Sequence
  VH CDR2 sequence for Group III antibody R29-8-57B
INYSGST

SEQ ID NO: 114 - protein, 7 aa, Artificial Sequence
  VH CDR2 sequence for Group III antibody R29-8-136C
INYSGSS

SEQ ID NO: 115 - protein, 7 aa, Artificial Sequence
  VH CDR2 sequence for Group III antibody R29-7-1C
ISYSGSI

SEQ ID NO: 116 - protein, 7 aa, Artificial Sequence
  VH CDR2 sequence for Group III antibody R29-67-7A
INYSGRT

SEQ ID NO: 117 - protein, 7 aa, Artificial Sequence
  VH CDR2 sequence for Group III antibody R29-67-4A
ISYSGIT

SEQ ID NO: 118 - protein, 14 aa, Artificial Sequence
  VH CDR3 sequence for Group III antibody R29-8-57B
ARERYDGVYYGMDY

SEQ ID NO: 119 - protein, 14 aa, Artificial Sequence
  VH CDR3 sequence for Group III antibody R29-7-1C
AREKYDNYYYAMDY

SEQ ID NO: 120 - protein, 14 aa, Artificial Sequence
  VH CDR3 sequence for Group III antibody R29-67-7A
ARENYDYEFYAMDY

SEQ ID NO: 121 - protein, 6 aa, Artificial Sequence
  VH CDR3 sequence for Group III antibody R29-67-4A
ATTGTR

SEQ ID NO: 122 - protein, 12 aa, Artificial Sequence
  VL CDR1 sequence for Group III antibody R29-7-1C
QNLLYSTNQKNY

SEQ ID NO: 123 - protein, 12 aa, Artificial Sequence
  VL CDR1 sequence for Group III antibody R29-67-7A
QNLLYSSNQKNY

SEQ ID NO: 124 - protein, 8 aa, Artificial Sequence
  VL CDR3 sequence for Group III antibody R29-8-57B
QQYYSYRT

SEQ ID NO: 125 - protein, 8 aa, Artificial Sequence
  VL CDR3 sequence for Group III antibody R29-67-7A
QQYYIYRT

SEQ ID NO: 126 - protein, 8 aa, Artificial Sequence
  VH CDR1 sequence for Group IV antibody R29-7A-53A
GFSLTDYA

SEQ ID NO: 127 - protein, 8 aa, Artificial Sequence
  VH CDR1 sequence for Group IV antibody R29-7A-49A
GFSLTDYG

SEQ ID NO: 128 - protein, 7 aa, Artificial Sequence
  VH CDR2 sequence for Group IV antibody R29-7A-53A
IWGGGRT

SEQ ID NO: 129 - protein, 7 aa, Artificial Sequence
  VH CDR2 sequence for Group IV antibody R29-7A-49A
IWGGGGT

SEQ ID NO: 130 - protein, 11 aa, Artificial Sequence
  VH CDR3 sequence for Group IV antibody R29-7A-53A
AKHEEVSRFAY

SEQ ID NO: 131 - DNA, 345 bp, Artificial Sequence
  R29-8-93B heavy chain variable region
gaagtgaagctggtggagtctgggggaggcgtagtgaagcctggagggtccctgaaactctcctgtacagcctctggattcactttcagtagttatgccatgtcttggtttcgccagacttcagagatgaggctggagtgggtcgcatccattactggtggtggtggcacctactatccagacagtgtgaagggccgattcaccatctccagagatactgtcaggaacatcctatacctgcaaatgagaagtctgaggtctggggacacggccatatattactgtgcaagaggctgggacgagaatgactactggggccaaggcacccctctcacagtctcctca

SEQ ID NO: 132 - protein, 115 aa, Artificial Sequence
  R29-8-93B heavy chain variable region
EVKLVESGGGVVKPGGSLKLSCTASGFTFSSYAMSWFRQTSEMRLEWVASITGGGGTYYPDSVKGRFTISRDTVRNILYLQMRSLRSGDTAIYYCARGWDENDYWGQGTPLTVSS

SEQ ID NO: 133 - DNA, 345 bp, Artificial Sequence
  R29-8-51B heavy chain variable region
gaagtgaagctggtggagtctgggggagacatggtgaagcctggagggtccctgaaactctcctgtgcagcctctggattcactttcagtagctatgccatgtcttggtttcgccagacttcagagatgaggctggagtgggtcgcgtccattactggtggtggtggcacctactatccagacagtgtgaagggccgattcaccatctccagagatactgtcaggaacatcctgtacctgcaaatgagaagtctgaggtctggggacacggccatatattactgtgcaagaggctgggacgagaatgactactggggccaaggcaccactctcaccgtctcctca

SEQ ID NO: 134 - protein, 115 aa, Artificial Sequence
  R29-8-51B heavy chain variable region
EVKLVESGGDMVKPGGSLKLSCAASGFTFSSYAMSWFRQTSEMRLEWVASITGGGGTYYPDSVKGRFTISRDTVRNILYLQMRSLRSGDTAIYYCARGWDENDYWGQGTTLTVSS

SEQ ID NO: 135 - DNA, 345 bp, Artificial Sequence
  R29-8-30A heavy chain variable region
gaagtgaagctggtggagtctgggggaggcttagtgaagcctggagggtccctgaaactctcctgtgcagcctctggattcactttcagtaactatgccatgtcttggtttcgccagactccagagaagaggctggagtgggtcgcatccattactggtggtggtagcacctactatccagacagtgtgaagggccgattcatcatctccagagataatgccaggaacatcctgtacctgcaaatgaggagtctgaggtctgaggacacggccatgtattactgtgcaagaggctgggacgagaatgactactggggccaaggcaccactctcacagtctcctca

SEQ ID NO: 136 - protein, 115 aa, Artificial Sequence
  R29-8-30A heavy chain variable region
EVKLVESGGGLVKPGGSLKLSCAASGFTFSNYAMSWFRQTPEKRLEWVASITGGGSTYYPDSVKGRFIISRDNARNILYLQMRSLRSEDTAMYYCARGWDENDYWGQGTTLTVSS

SEQ ID NO: 137 - DNA, 345 bp, Artificial Sequence
  R29-8-18B heavy chain variable region
gaagtgaagctggtggagtctgggggaggcttagtgaagcctggagggtccctgaaactctcctgtgcagcctctggattcactttcagtacctatgccgtgtcttggtttcgccagactccagagaggaggctggagtgggtcgcatccattactagtggtggtagcacctactatccagacagtgtgaagggccgattcaccatctccagagataatgccaggaacatcctgtacctgcaaatgatcagtctgaggtctggggacacggccatgtattactgtgcaagaggctgggacgagaatgactactggggccaaggcaccactctcacagtctcctca

SEQ ID NO: 138 - protein, 115 aa, Artificial Sequence
  R29-8-18B heavy chain variable region
EVKLVESGGGLVKPGGSLKLSCAASGFTFSTYAVSWFRQTPERRLEWVASITSGGSTYYPDSVKGRFTISRDNARNILYLQMISLRSGDTAMYYCARGWDENDYWGQGTTLTVSS

SEQ ID NO: 139 - DNA, 351 bp, Artificial Sequence
  R29-7-38C heavy chain variable region
caggtgcagctgaaggagtcaggacctggcctggtggcgccctcacagagcctgtccatcacatgcactgtctcagggttttcattaaccgactatgccataagctggattcgccagcctccaggaaagggtctggagtggctgggagtaatatggggtggtggaagaacatattctaattcagctctcaaatccagactgagcatcagcaaggacaactccaggagccaaattttcttaaaaatgaacagtctgcaaactgatgacacagccatgtactactgtgccaaacatgaggaggtatcccgttttgctcactggggccaagggactctggtcactgtctctgca

SEQ ID NO: 140 - protein, 117 aa, Artificial Sequence
  R29-7-38C heavy chain variable region
QVQLKESGPGLVAPSQSLSITCTVSGFSLTDYAISWIRQPPGKGLEWLGVIWGGGRTYSNSALKSRLSISKDNSRSQIFLKMNSLQTDDTAMYYCAKHEEVSRFAHWGQGTLVTVSA

SEQ ID NO: 141 - DNA, 351 bp, Artificial Sequence
  R29-7-49A heavy chain variable region
caggtgcagctgaaggagtcaggacctggcctggtggcgccctcacagagcctgtccatcacatgcactgtctcagggttctcattaaccgactatggtgtaagctggattcgccagcctccaggaaagggtctggagtggctgggagtaatatggggtggtggaggcacatactataattcagctctcaaatccagactgagcatcaacaaggacaactccaagagccaaattttcttaaaaatgaacagtctgcaaactgatgacacagccatttactactgtgccaaacatgaggaggtatcccggtttgcttactggggccaagggactctggtcactgtctctgca

SEQ ID NO: 142 - protein, 117 aa, Artificial Sequence
  R29-7-49A heavy chain variable region
QVQLKESGPGLVAPSQSLSITCTVSGFSLTDYGVSWIRQPPGKGLEWLGVIWGGGGTYYNSALKSRLSINKDNSKSQIFLKMNSLQTDDTAIYYCAKHEEVSRFAYWGQGTLVTVSA

SEQ ID NO: 143 - DNA, 351 bp, Artificial Sequence
  R29-7-13A heavy chain variable region
caggtgcagctgaaggagtcaggacctggcctggtggcgccctcacagagcctgtccatcacatgcactgtctcagggttctcattaaccgactatgccataagctggattcgccagcctccaggaaagggtctggagtggctgggagtaatatggggtggtggaagaacatactctaattcagctctcaaatccagactgagcatcagcaaggacaattccaggagccaaattttcttaaaaatgaacagtctgcacactgaagacacagccgtgtactactgtgccaaacatgaggaggtctcccgttttgcttactggggccaagggactctggtcactgtctctgca

SEQ ID NO: 144 - protein, 117 aa, Artificial Sequence
  R29-7-13A heavy chain variable region
QVQLKESGPGLVAPSQSLSITCTVSGFSLTDYAISWIRQPPGKGLEWLGVIWGGGRTYSNSALKSRLSISKDNSRSQIFLKMNSLHTEDTAVYYCAKHEEVSRFAYWGQGTLVTVSA

SEQ ID NO: 145 - DNA, 342 bp, Artificial Sequence
  R29-67-4A heavy chain variable region
tctgatgtgcaacttcaggagtcgggacctggcctggtgaaaccttctcagtctctgtccctcacctgcactgtcactggctactcaatcaccagtgtttatgcctggaactggatccggcagtttccaggaaacaaactggagtggatgggctacataagctacagtggtatcactacctacaacccatctctcaaaagtcgaatctctatcactcgagacacatccaagaaccagttcttcctgcagttgaattctgtgactactgaggacacagccacatattactgtgcaacaactgggacccgctggggccaaggcaccactctcacagtctcctca

SEQ ID NO: 146 - protein, 114 aa, Artificial Sequence
  R29-67-4A heavy chain variable region
SDVQLQESGPGLVKPSQSLSLTCTVTGYSITSVYAWNWIRQFPGNKLEWMGYISYSGITTYNPSLKSRISITRDTSKNQFFLQLNSVTTEDTATYYCATTGTRWGQGTTLTVSS

SEQ ID NO: 147 - protein, 5 aa, Artificial Sequence
  VH CDR1 of Antibody R29-8-9B - Kabat
SYAMS

SEQ ID NO: 148 - protein, 16 aa, Artificial Sequence
  VH CDR2 of Antibody R29-8-9B - Kabat
SITGGGGTYYPDSVKG

SEQ ID NO: 149 - protein, 7 aa, Artificial Sequence
  VH CDR3 of Antibody R29-8-9B - Kabat
GWDENDY

SEQ ID NO: 150 - protein, 16 aa, Artificial Sequence
  VL CDR1 of Antibody R29-8-9B - Kabat
RSSQSLVFSNGNTYLH

SEQ ID NO: 151 - protein, 17 aa, Artificial Sequence
  SIT1 peptide as negative control
DNCTDLLALGIPSITQK

SEQ ID NO: 152 - protein, 31 aa, Artificial Sequence
  C16 peptide as posotive control
MPLTPEPPSGRVEGPPAWEAAPWPSLPCGPC

SEQ ID NO: 153 - protein, 31 aa, Homo sapiens; identical to SEQ ID NO: 152
  Human C16-ECD

SEQ ID NO: 154 - protein, 31 aa, Macaca fascicularis
  Cyno C16-ECD
MPSTPEPPSGRMEGPPTWEAAPWPSLPCGPC

SEQ ID NO: 155 - protein, 6 aa, Artificial Sequence
  6 His Tag
HHHHHH

SEQ ID NO: 156 - protein, 6 aa, Artificial Sequence
  Thrombin site
LVPRGS

SEQ ID NO: 157 - protein, 9 aa, Artificial Sequence
  IgG1 Hinge region
DKTHTCPPC

SEQ ID NO: 158 - protein, 218 aa, Artificial Sequence
  CH3 CH4 domains
PAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 159 - protein, 22 aa, Mus musculus
  Mouse kappa signal peptide
MDFQVQIFSFLLISASVIMSRG

SEQ ID NO: 160 - protein, 7 aa, Artificial Sequence
  VL CDR3 of Antibody R29-8-9B - Kabat
KVSNRFS

SEQ ID NO: 161 - protein, 7 aa, Artificial Sequence
  VH CDR1 of Antibody R29-8-9B - Chothia
GFTFSSY

SEQ ID NO: 162 - protein, 3 aa, Artificial Sequence
  VH CDR2 of Antibody R29-8-9B - Chothia
GGG

SEQ ID NO: 163 - protein, 5 aa, Artificial Sequence
  VH CDR3 of Antibody R29-8-9B - Chothia
WDEND

SEQ ID NO: 164 - protein, 12 aa, Artificial Sequence
  VL CDR1 of Antibody R29-8-9B - Chothia
SQSLVFSNGNTY

SEQ ID NO: 165 - protein, 6 aa, Artificial Sequence
  VL CDR3 of Antibody R29-8-9B - Chothia
STHVPW

SEQ ID NO: 166 - protein, 6 aa, Artificial Sequence
  VH CDR1 of Antibody R29-8-9B - Contact
SSYAMS

SEQ ID NO: 167 - protein, 12 aa, Artificial Sequence
  VH CDR2 of Antibody R29-8-9B - Contact
WVASITGGGGTY

SEQ ID NO: 168 - protein, 8 aa, Artificial Sequence
  VH CDR3 of Antibody R29-8-9B - Contact
ARGWDEND

SEQ ID NO: 169 - protein, 12 aa, Artificial Sequence
  VL CDR1 of Antibody R29-8-9B - Contact
VFSNGNTYLHWY

SEQ ID NO: 170 - protein, 10 aa, Artificial Sequence
  VL CDR2 of Antibody R29-8-9B - Contact
LLIYKVSNRF

SEQ ID NO: 171 - protein, 8 aa, Artificial Sequence
  VL CDR3 of Antibody R29-8-9B - Contact
SQSTHVPW

SEQ ID NO: 172 - protein, 10 aa, Artificial Sequence
  VH CDR1 of Antibody R29-8-9B - AbM
GFTFSSYAMS

SEQ ID NO: 173 - protein, 9 aa, Artificial Sequence
  VH CDR2 of Antibody R29-8-9B - AbM
SITGGGGTY

SEQ ID NO: 174 - protein, 5 aa, Artificial Sequence
  VH CDR1 of Antibody R29-8-30A - Kabat
NYAMS

SEQ ID NO: 175 - protein, 16 aa, Artificial Sequence
  VH CDR2 of Antibody R29-8-30A - Kabat
SITGGGSTYYPDSVKG

SEQ ID NO: 176 - protein, 7 aa, Artificial Sequence
  VH CDR1 of Antibody R29-8-30A - Chothia
GFTFSNY

SEQ ID NO: 177 - protein, 6 aa, Artificial Sequence
  VH CDR1 of Antibody R29-8-30A - Contact
SNYAMS

SEQ ID NO: 178 - protein, 12 aa, Artificial Sequence
  VH CDR2 of Antibody R29-8-30A - Contact
WVASITGGGSTY

SEQ ID NO: 179 - protein, 10 aa, Artificial Sequence
  VH CDR1 of Antibody R29-8-30A - AbM
GFTFSNYAMS

SEQ ID NO: 180 - protein, 9 aa, Artificial Sequence
  VH CDR2 of Antibody R29-8-30A - AbM
SITGGGSTY

SEQ ID NO: 181 - protein, 5 aa, Artificial Sequence
  VH CDR1 of Antibody R29-8-120B - Kabat
IYAMS

SEQ ID NO: 182 - protein, 16 aa, Artificial Sequence
  VH CDR2 of Antibody R29-8-120B - Kabat
SITGGGTNFYPDSVRG

SEQ ID NO: 183 - protein, 16 aa, Artificial Sequence
  VL CDR1 of Antibody R29-8-120B - Kabat
RSSQSLVYSNGNTYLH

SEQ ID NO: 184 - protein, 7 aa, Artificial Sequence
  VH CDR1 of Antibody R29-8-120B - Chothia
GFTFSIY

SEQ ID NO: 185 - protein, 12 aa, Artificial Sequence
  VL CDR1 of Antibody R29-8-120B - Chothia
SQSLVYSNGNTY

SEQ ID NO: 186 - protein, 6 aa, Artificial Sequence
  VH CDR1 of Antibody R29-8-120B - Contact
SIYAMS

SEQ ID NO: 187 - protein, 12 aa, Artificial Sequence
  VH CDR2 of Antibody R29-8-120B - Contact
WVASITGGGTNF

SEQ ID NO: 188 - protein, 12 aa, Artificial Sequence
  VL CDR1 of Antibody R29-8-120B - Contact
VYSNGNTYLHWY

SEQ ID NO: 189 - protein, 10 aa, Artificial Sequence
  VH CDR1 of Antibody R29-8-120B - AbM
GFTFSIYAMS

SEQ ID NO: 190 - protein, 9 aa, Artificial Sequence
  VH CDR2 of Antibody R29-8-120B - AbM
SITGGGTNF

SEQ ID NO: 191 - protein, 5 aa, Artificial Sequence
  VH CDR1 of Antibody R29-8-18B - Kabat
TYAVS

SEQ ID NO: 192 - protein, 16 aa, Artificial Sequence
  VH CDR2 of Antibody R29-8-18B - Kabat
SITSGGSTYYPDSVKG

SEQ ID NO: 193 - protein, 7 aa, Artificial Sequence
  VH CDR1 of Antibody R29-8-18B - Chothia
GFTFSTY

SEQ ID NO: 194 - protein, 3 aa, Artificial Sequence
  VH CDR2 of Antibody R29-8-18B - Chothia
SGG

SEQ ID NO: 195 - protein, 6 aa, Artificial Sequence
  VH CDR1 of Antibody R29-8-18B - Contact
STYAVS

SEQ ID NO: 196 - protein, 12 aa, Artificial Sequence
  VH CDR2 of Antibody R29-8-18B - Contact
WVASITSGGSTY

SEQ ID NO: 197 - protein, 10 aa, Artificial Sequence
  VH CDR1 of Antibody R29-8-18B - AbM
GFTFSTYAVS

SEQ ID NO: 198 - protein, 9 aa, Artificial Sequence
  VH CDR2 of Antibody R29-8-18B - AbM
SITSGGSTY

SEQ ID NO: 199 - protein, 5 aa, Artificial Sequence
  VH CDR1 of Antibody R29-8-28C - Kabat
NYAVS

SEQ ID NO: 200 - protein, 16 aa, Artificial Sequence
  VH CDR2 of Antibody R29-8-28C - Kabat
SITSGGRTYYPDSMKG

SEQ ID NO: 201 - protein, 6 aa, Artificial Sequence
  VL CDR3 of Antibody R29-8-28C - Chothia
TTHVPW

SEQ ID NO: 202 - protein, 6 aa, Artificial Sequence
  VH CDR1 of Antibody R29-8-28C - Contact
SNYAVS

SEQ ID NO: 203 - protein, 12 aa, Artificial Sequence
  VH CDR2 of Antibody R29-8-28C - Contact
WVASITSGGRTY

SEQ ID NO: 204 - protein, 8 aa, Artificial Sequence
  VH CDR3 of Antibody R29-8-28C - Contact
GRGWDEND

SEQ ID NO: 205 - protein, 8 aa, Artificial Sequence
  VL CDR3 of Antibody R29-8-28C - Contact
SQTTHVPW

SEQ ID NO: 206 - protein, 10 aa, Artificial Sequence
  VH CDR1 of Antibody R29-8-28C - AbM
GFTFSNYAVS

SEQ ID NO: 207 - protein, 9 aa, Artificial Sequence
  VH CDR2 of Antibody R29-8-28C - AbM
SITSGGRTY

SEQ ID NO: 208 - protein, 5 aa, Artificial Sequence
  VH CDR1 of Antibody R29-8-19B - Kabat
SYAVS

SEQ ID NO: 209 - protein, 16 aa, Artificial Sequence
  VH CDR2 of Antibody R29-8-19B - Kabat
SITSGGRTYYPDSVKG

SEQ ID NO: 210 - protein, 7 aa, Artificial Sequence
  VH CDR3 of Antibody R29-8-19B - Kabat
GWDENDL

SEQ ID NO: 211 - protein, 6 aa, Artificial Sequence
  VH CDR1 of Antibody R29-8-19B - Contact
SSYAVS

SEQ ID NO: 212 - protein, 8 aa, Artificial Sequence
  VH CDR3 of Antibody R29-8-19B - Contact
TRGWDEND

SEQ ID NO: 213 - protein, 10 aa, Artificial Sequence
  VH CDR1 of Antibody R29-8-19B - AbM
GFTFSSYAVS

SEQ ID NO: 214 - protein, 16 aa, Artificial Sequence
  VH CDR2 of Antibody R29-8-36C - Kabat
SITSGGRSYYPDSVKG

SEQ ID NO: 215 - protein, 16 aa, Artificial Sequence
  VL CDR1 of Antibody R29-8-36C - Kabat
RSSQSLLYSNGNTYLH

SEQ ID NO: 216 - protein, 12 aa, Artificial Sequence
  VL CDR1 of Antibody R29-8-36C - Chothia
SQSLLYSNGNTY

SEQ ID NO: 217 - protein, 12 aa, Artificial Sequence
  VL CDR1 of Antibody R29-8-36C - Contact
LYSNGNTYLHWY

SEQ ID NO: 218 - protein, 9 aa, Artificial Sequence
  VH CDR2 of Antibody R29-8-36C - AbM
SITSGGRSY

SEQ ID NO: 219 - protein, 5 aa, Artificial Sequence
  VH CDR1 of Antibody R29-8-75B - Kabat
RFGMH

SEQ ID NO: 220 - protein, 17 aa, Artificial Sequence
  VH CDR2 of Antibody R29-8-75B - Kabat
YISSGSSTIYYADTVKG

SEQ ID NO: 221 - protein, 9 aa, Artificial Sequence
  VH CDR3 of Antibody R29-8-75B - Kabat
VDYDVALAY

SEQ ID NO: 222 - protein, 16 aa, Artificial Sequence
  VL CDR1 of Antibody R29-8-75B - Kabat
RSSQSIVHRNGNTYLE

SEQ ID NO: 223 - protein, 16 aa, Artificial Sequence
  VH CDR2 of Antibody R29-8-136C - Kabat
YINYSGSSRYNPSLKS

SEQ ID NO: 224 - protein, 7 aa, Artificial Sequence
  VH CDR1 of Antibody R29-8-75B - Chothia
GFTFSRF

SEQ ID NO: 225 - protein, 4 aa, Artificial Sequence
  VH CDR2 of Antibody R29-8-75B - Chothia
SGSS

SEQ ID NO: 226 - protein, 7 aa, Artificial Sequence
  VH CDR3 of Antibody R29-8-75B - Chothia
DYDVALA

SEQ ID NO: 227 - protein, 12 aa, Artificial Sequence
  VL CDR1 of Antibody R29-8-75B - Chothia
SQSIVHRNGNTY

SEQ ID NO: 228 - protein, 4 aa, Artificial Sequence
  VL CDR3 of Antibody R29-8-75B - Chothia
GSQW

SEQ ID NO: 229 - protein, 6 aa, Artificial Sequence
  VH CDR1 of Antibody R29-8-75B - Contact
SRFGMH

SEQ ID NO: 230 - protein, 13 aa, Artificial Sequence
  VH CDR2 of Antibody R29-8-75B - Contact
WVAYISSGSSTIY

SEQ ID NO: 231 - protein, 10 aa, Artificial Sequence
  VH CDR3 of Antibody R29-8-75B - Contact
ARVDYDVALA

SEQ ID NO: 232 - protein, 12 aa, Artificial Sequence
  VL CDR1 of Antibody R29-8-75B - Contact
VHRNGNTYLEWY

SEQ ID NO: 233 - protein, 6 aa, Artificial Sequence
  VL CDR3 of Antibody R29-8-75B - Contact
FQGSQW

SEQ ID NO: 234 - protein, 6 aa, Artificial Sequence
  VH CDR1 of Antibody R29-8-57B - Kabat
SDYAWN

SEQ ID NO: 235 - protein, 16 aa, Artificial Sequence
  VH CDR2 of Antibody R29-8-57B - Kabat
YINYSGSTRYNPSLKS

SEQ ID NO: 236 - protein, 12 aa, Artificial Sequence
  VH CDR3 of Antibody R29-8-57B - Kabat
ERYDGVYYGMDY

SEQ ID NO: 237 - protein, 17 aa, Artificial Sequence
  VL CDR1 of Antibody R29-8-57B - Kabat
KSSQSLLYSSNQKNYLA

SEQ ID NO: 238 - protein, 7 aa, Artificial Sequence
  VL CDR2 of Antibody R29-8-57B - Kabat
WASTRES

SEQ ID NO: 239 - protein, 3 aa, Artificial Sequence
  VH CDR2 of Antibody R29-8-57B - Chothia
YSG

SEQ ID NO: 240 - protein, 8 aa, Artificial Sequence
  VH CDR1 of Antibody R29-8-57B - Chothia
GYSITSDY

SEQ ID NO: 241 - protein, 10 aa, Artificial Sequence
  VH CDR3 of Antibody R29-8-57B - Chothia
RYDGVYYGMD

SEQ ID NO: 242 - protein, 13 aa, Artificial Sequence
  VL CDR1 of Antibody R29-8-57B - Chothia
SQSLLYSSNQKNY

SEQ ID NO: 243 - protein, 5 aa, Artificial Sequence
  VL CDR3 of Antibody R29-8-57B - Chothia
YYSYR

SEQ ID NO: 244 - protein, 7 aa, Artificial Sequence
  VH CDR1 of Antibody R29-8-57B - Contact
TSDYAWN

SEQ ID NO: 245 - protein, 12 aa, Artificial Sequence
  VH CDR2 of Antibody R29-8-57B - Contact
WMGYINYSGSTR

SEQ ID NO: 246 - protein, 13 aa, Artificial Sequence
  VH CDR3 of Antibody R29-8-57B - Contact
ARERYDGVYYGMD

SEQ ID NO: 247 - protein, 13 aa, Artificial Sequence
  VL CDR1 of Antibody R29-8-57B - Contact
LYSSNQKNYLAWY

SEQ ID NO: 248 - protein, 10 aa, Artificial Sequence
  VL CDR2 of Antibody R29-8-57B - Contact
LLIYWASTRE

SEQ ID NO: 249 - protein, 7 aa, Artificial Sequence
  VL CDR3 of Antibody R29-8-57B - Contact
QQYYSYR

SEQ ID NO: 250 - protein, 11 aa, Artificial Sequence
  VH CDR1 of Antibody R29-8-57B - AbM
GYSITSDYAWN

SEQ ID NO: 251 - protein, 9 aa, Artificial Sequence
  VH CDR2 of Antibody R29-8-57B - AbM
YINYSGSTR

SEQ ID NO: 252 - protein, 12 aa, Artificial Sequence
  VH CDR2 of Antibody R29-8-136C - Contact
WMGYINYSGSSR

SEQ ID NO: 253 - protein, 9 aa, Artificial Sequence
  VH CDR2 of Antibody R29-8-136C - AbM
YINYSGSSR

SEQ ID NO: 254 - protein, 16 aa, Artificial Sequence
  VH CDR2 of Antibody R29-7-1C - Kabat
YISYSGSIRYNPSLKS

SEQ ID NO: 255 - protein, 12 aa, Artificial Sequence
  VH CDR3 of Antibody R29-7-1C - Kabat
EKYDNYYYAMDY

SEQ ID NO: 256 - protein, 17 aa, Artificial Sequence
  VL CDR1 of Antibody R29-7-1C - Kabat
KSSQNLLYSTNQKNYLA

SEQ ID NO: 257 - protein, 10 aa, Artificial Sequence
  VH CDR3 of Antibody R29-7-1C - Chothia
KYDNYYYAMD

SEQ ID NO: 258 - protein, 13 aa, Artificial Sequence
  VL CDR1 of Antibody R29-7-1C - Chothia
SQNLLYSTNQKNY

SEQ ID NO: 259 - protein, 12 aa, Artificial Sequence
  VH CDR2 of Antibody R29-7-1C - Contact
WMGYISYSGSIR

SEQ ID NO: 260 - protein, 13 aa, Artificial Sequence
  VH CDR3 of Antibody R29-7-1C - Contact
AREKYDNYYYAMD

SEQ ID NO: 261 - protein, 13 aa, Artificial Sequence
  VL CDR1 of Antibody R29-7-1C - Contact
LYSTNQKNYLAWY

SEQ ID NO: 262 - protein, 9 aa, Artificial Sequence
  VH CDR2 of Antibody R29-7-1C - AbM
YISYSGSIR

SEQ ID NO: 263 - protein, 16 aa, Artificial Sequence
  VH CDR2 of Antibody R29-67-7A - Kabat
YINYSGRTRYNPSLNS

SEQ ID NO: 264 - protein, 12 aa, Artificial Sequence
  VH CDR3 of Antibody R29-67-7A - Kabat
ENYDYEFYAMDY

SEQ ID NO: 265 - protein, 17 aa, Artificial Sequence
  VL CDR1 of Antibody R29-67-7A - Kabat
KSSQNLLYSSNQKNYLA

SEQ ID NO: 266 - protein, 8 aa, Artificial Sequence
  VH CDR1 of Antibody R29-67-4A - Chothia
GYSITSVY

SEQ ID NO: 267 - protein, 10 aa, Artificial Sequence
  VH CDR3 of Antibody R29-67-4A - Chothia
NYDYEFYAMD

SEQ ID NO: 268 - protein, 13 aa, Artificial Sequence
  VL CDR1 of Antibody R29-67-7A - Chothia
SQNLLYSSNQKNY

SEQ ID NO: 269 - protein, 5 aa, Artificial Sequence
  VL CDR3 of Antibody R29-67-7A - Chothia
YYIYR

SEQ ID NO: 270 - protein, 12 aa, Artificial Sequence
  VH CDR2 of Antibody R29-67-7A - Contact
WMGYINYSGRTR

SEQ ID NO: 271 - protein, 13 aa, Artificial Sequence
  VH CDR3 of Antibody R29-67-7A - Contact
ARENYDYEFYAMD

SEQ ID NO: 272 - protein, 7 aa, Artificial Sequence
  VL CDR3 of Antibody R29-67-7A - Contact
QQYYIYR

SEQ ID NO: 273 - protein, 9 aa, Artificial Sequence
  VH CDR2 of Antibody R29-67-7A - AbM
YINYSGRTR

SEQ ID NO: 274 - protein, 6 aa, Artificial Sequence
  VH CDR1 of Antibody R29-67-4A - Kabat
SVYAWN

SEQ ID NO: 275 - protein, 16 aa, Artificial Sequence
  VH CDR2 of Antibody R29-67-4A - Kabat
YISYSGITTYNPSLKS

SEQ ID NO: 276 - protein, 4 aa, Artificial Sequence
  VH CDR3 of Antibody R29-67-4A - Kabat
TGTR

SEQ ID NO: 277 - protein, 7 aa, Artificial Sequence
  VH CDR1 of Antibody R29-67-4A - Contact
TSVYAWN

SEQ ID NO: 278 - protein, 12 aa, Artificial Sequence
  VH CDR2 of Antibody R29-67-4A - Contact
WMGYISYSGITT

SEQ ID NO: 279 - protein, 11 aa, Artificial Sequence
  VH CDR1 of Antibody R29-67-4A - AbM
GYSITSVYAWN

SEQ ID NO: 280 - protein, 9 aa, Artificial Sequence
  VH CDR2 of Antibody R29-67-4A - AbM
YISYSGITT

SEQ ID NO: 281 - protein, 5 aa, Artificial Sequence
  VH CDR1 of Antibody R29-7A-53A - Kabat
DYAIS

SEQ ID NO: 282 - protein, 16 aa, Artificial Sequence
  VH CDR2 of Antibody R29-7A-53A - Kabat
VIWGGGRTYSNSALKS

SEQ ID NO: 283 - protein, 9 aa, Artificial Sequence
  VH CDR3 of Antibody R29-7A-53A - Kabat
HEEVSRFAY

SEQ ID NO: 284 - protein, 16 aa, Artificial Sequence
  VL CDR1 of Antibody R29-7A-53A - Kabat
RSSQSLVYSNGNSYLH

SEQ ID NO: 285 - protein, 7 aa, Artificial Sequence
  VH CDR1 of Antibody R29-7A-53A - Chothia
GFSLTDY

SEQ ID NO: 286 - protein, 7 aa, Artificial Sequence
  VH CDR3 of Antibody R29-7A-53A - Chothia
EEVSRFA

SEQ ID NO: 287 - protein, 12 aa, Artificial Sequence
  VL CDR1 of Antibody R29-7A-53A - Chothia
SQSLVYSNGNSY

SEQ ID NO: 288 - protein, 6 aa, Artificial Sequence
  VL CDR3 of Antibody R29-7A-53A - Chothia
STHIPL

SEQ ID NO: 289 - protein, 6 aa, Artificial Sequence
  VH CDR1 of Antibody R29-7A-53A - Contact
TDYAIS

SEQ ID NO: 290 - protein, 12 aa, Artificial Sequence
  VH CDR2 of Antibody R29-7A-53A - Contact
WLGVIWGGGRTY

SEQ ID NO: 291 - protein, 10 aa, Artificial Sequence
  VH CDR3 of Antibody R29-7A-53A - Contact
AKHEEVSRFA

SEQ ID NO: 292 - protein, 12 aa, Artificial Sequence
  VL CDR1 of Antibody R29-7A-53A - Contact
VYSNGNSYLHWY

SEQ ID NO: 293 - protein, 8 aa, Artificial Sequence
  VL CDR3 of Antibody R29-7A-53A - Contact
SQSTHIPL

SEQ ID NO: 294 - protein, 10 aa, Artificial Sequence
  VH CDR1 of Antibody R29-7A-53A - AbM
GFSLTDYAIS

SEQ ID NO: 295 - protein, 9 aa, Artificial Sequence
  VH CDR2 of Antibody R29-7A-53A - AbM
VIWGGGRTY

SEQ ID NO: 296 - protein, 9 aa, Artificial Sequence
  VH CDR3 of Antibody R29-7A-38C - Kabat
HEEVSRFAH

SEQ ID NO: 297 - protein, 5 aa, Artificial Sequence
  VL CDR1 of Antibody R29-7A-38C - Kabat
DYGVS

SEQ ID NO: 298 - protein, 16 aa, Artificial Sequence
  VL CDR2 of Antibody R29-7A-38C - Kabat
VIWGGGGTYYNSALKS

SEQ ID NO: 299 - protein, 6 aa, Artificial Sequence
  VL CDR1 of Antibody R29-7A-38C - Contact
TDYGVS

SEQ ID NO: 300 - protein, 12 aa, Artificial Sequence
  VL CDR2 of Antibody R29-7A-38C - Contact
WLGVIWGGGGTY

SEQ ID NO: 301 - protein, 10 aa, Artificial Sequence
  VL CDR1 of Antibody R29-7A-38C - AbM
GFSLTDYGVS

SEQ ID NO: 302 - protein, 9 aa, Artificial Sequence
  VL CDR2 of Antibody R29-7A-38C - AbM
VIWGGGGTY

SEQ ID NO: 303 - protein, 5 aa, Artificial Sequence
  VH CDR1 of Antibody R29-7-2A - Kabat
THAMN

SEQ ID NO: 304 - protein, 19 aa, Artificial Sequence
  VH CDR2 of Antibody R29-7-2A - Kabat
RIRSKSNNYARYYADSVKD

SEQ ID NO: 305 - protein, 8 aa, Artificial Sequence
  VH CDR3 of Antibody R29-7-2A - Kabat
QGDGGFAY

SEQ ID NO: 306 - protein, 7 aa, Artificial Sequence
  VL CDR2 of Antibody R29-7-2A - Kabat
WASARES

SEQ ID NO: 307 - protein, 7 aa, Artificial Sequence
  VH CDR1 of Antibody R29-7-2A - Chothia
GFSFNTH

SEQ ID NO: 308 - protein, 6 aa, Artificial Sequence
  VH CDR2 of Antibody R29-7-2A - Chothia
SKSNNY

SEQ ID NO: 309 - protein, 6 aa, Artificial Sequence
  VH CDR3 of Antibody R29-7-2A - Chothia
GDGGFA

SEQ ID NO: 310 - protein, 6 aa, Artificial Sequence
  VL CDR3 of Antibody R29-7-2A - Chothia
YYSYPP

SEQ ID NO: 311 - protein, 6 aa, Artificial Sequence
  VH CDR1 of Antibody R29-7-2A - Contact
NTHAMN

SEQ ID NO: 312 - protein, 15 aa, Artificial Sequence
  VH CDR2 of Antibody R29-7-2A - Contact
WVARIRSKSNNYARY

SEQ ID NO: 313 - protein, 9 aa, Artificial Sequence
  VH CDR3 of Antibody R29-7-2A - Contact
VKQGDGGFA

SEQ ID NO: 314 - protein, 10 aa, Artificial Sequence
  VL CDR2 of Antibody R29-7-2A - Contact
LLIYWASARE

SEQ ID NO: 315 - protein, 8 aa, Artificial Sequence
  VL CDR3 of Antibody R29-7-2A - Contact
QQYYSYPP

SEQ ID NO: 316 - protein, 12 aa, Artificial Sequence
  VH CDR2 of Antibody R29-7-2A - AbM
RIRSKSNNYARY

SEQ ID NO: 317 - protein, 10 aa, Artificial Sequence
  VH CDR1 of Antibody R29-7-2A - AbM
GFSFNTHAMN

SEQ ID NO: 318 - protein, 224 aa, Macaca fascicularis
MPSTPEPPSGRMEGPPTWEAAPWPSLPCGPCIPIMLALATLAALFILTTAVLAERLFRRALRPDPSHHAPTLVWRPGGELWIEPMGTPRERSEDWYGSAVPLLTDRAPEPPTPVGVLEAQATAPYAPSAPNSAPSSLGPQTPPEVPARSTFWGPQPWEGRPPGTGLVSWAEPEQRPEARAQFGSPQARRQRPRSPDPEWGLQPRVTLEQISAFWRREGRTSVGF

SEQ ID NO: 319 - protein, 225 aa, Mus musculus
MPVTPQQPSGHTEGLPEPTAEAAVWVVIPCGPCIPIMLGLASLTAFFIITTAVLAERLFRRPQPDPSQRAPTLVWRPGGELWIEPTSSARERSEDWYGSSMPLLMDRAPGPPTPGGTLEGRATAPPATSAPYSSLSSLVPQTPPEVPAQSTFWRPQTQEERPHDTSLVSWVGSEPMPEAGLQVGSPRPWRPRQGSLEPDWGLQPRVTLEQISAFWKREGRTSVGF